(19) United States
(12) Patent Application Publication
(10) Pub. No.: US 2026/0232791 A1
(43) Pub. Date: Aug. 13, 2026

(54) PHARMACEUTICAL COMPOSITIONS FOR DELIVERY OF HERPES SIMPLEX VIRUS GLYCOPROTEIN C, GLYCOPROTEIN D, AND GLYCOPROTEIN E ANTIGENS AND RELATED METHODS

(71) Applicant: BioNTech SE, Mainz (DE)

(72) Inventors: Alptekin Güler, Mainz (DE); Ricardo Sanchez Velazquez, Mainz (DE); Julia Uebele, Darmstadt (DE); Annette Vogel, Mainz (DE); Christina Pfafenrot, Mainz (DE); Anna Luise Ernst, Mainz (DE); Stephanie Hein, Rüsselsheim (DE); Sabrina Hinz, Wörrstadt (DE); Sarah Catharina Dany, Weiterstadt (DE)

(21) Appl. No.: 19/151,231

(22) PCT Filed: Jan. 26, 2024
(86) PCT No.: PCT/IB24/50751

§ 371 Date: Jul. 25, 2025

Related U.S. Application Data
(60) Provisional application No. 63/441,767, filed on Jan. 27, 2023.
(60) Provisional application No. 63/517,380, filed on Aug. 3, 2023.
(60) Provisional application No. 63/594,825, filed on Oct. 31, 2023.

(51) Int. Cl. A61K 39/245; A61K 39/00; A61K 39/295; A61P 31/22

(52) U.S. Cl. CPC A61K 39/245; A61K 39/295; A61P 31/22; A61K 2039/53; A61K 2039/55555; C12N 2710/16622; C12N 2710/16634

(57) ABSTRACT

The present disclosure provides pharmaceutical compositions for delivery of HSV antigens (e.g., an HSV vaccine) and related technologies (e.g., components thereof and/or methods relating thereto).

The present application claims priority to and the benefit of U.S. Provisional Patent Application No. 63/441,767 filed on Jan. 27, 2023, U.S. Provisional Patent Application No. 63/517,380 filed on Aug. 3, 2023, and U.S. Provisional Patent Application No. 63/594,825 filed on Oct. 31, 2023, the entirety of which are incorporated herein by reference.

### BACKGROUND

Herpes simplex viruses (HSV), commonly referred to only as herpes, are categorized into two types: herpes simplex virus, type 1 (HSV-1, or oral herpes) and herpes simplex virus, type 2 (HSV-2, or genital herpes). According to the World Health Organization, an estimated 3.7 billion people under age 50 (67% of global population) have HSV-1 infection globally. HSV-1 prevalence is understood as being highest in Africa and lowest in the Americas. An estimated 491 million people aged 15-49 (13% of global population) worldwide have HSV-2 infection. More women are infected with HSV-2 than men, because sexual transmission of HSV is more efficient from men to women than from women to men. Prevalence of HSV-2 infection was estimated to be highest in Africa, followed by the Americas. Prevalence of HSV-2 was also shown to increase with age, though the highest numbers of people newly-infected have historically been in adolescents. Both HSV-1 and HSV-2 infections are lifelong.

### SUMMARY

The present disclosure provides pharmaceutical compositions (e.g., immunogenic compositions, e.g., vaccines) for delivering particular herpes simplex virus (HSV) antigen constructs to a subject (e.g., a patient) and related technologies (e.g., methods). In particular, the present disclosure provides HSV vaccine compositions and related technologies (e.g., methods).

The present disclosure also provides that HSV glycoprotein C (gC) antigens or antigenic fragments thereof, HSV glycoprotein D (gD) antigens or antigenic fragments thereof, glycoprotein E (gE) antigens or antigenic fragments thereof, or combinations thereof can be useful in preventing or treating HSV, e.g., in HSV antigen constructs and/or HSV vaccines as further disclosed herein.

The present disclosure provides, for example, polyribonucleotides that encodes one or more HSV antigens or antigenic fragments thereof. In some embodiments, polyribonucleotides described herein encode one or more of HSV-2 gC, gD, and/or gE antigens or antigenic fragments thereof (e.g., in a construct). In some embodiments, such a polyribonucleotide can be part of an RNA construct. In some embodiments, a polyribonucleotide or RNA construct as described herein can be part of a composition (e.g., a pharmaceutical composition, e.g., an immunogenic composition, e.g., a vaccine.

In some embodiments, technologies provided herein are directed against HSV.

### BRIEF DESCRIPTION OF THE DRAWING

The Drawing included herein, which is composed of the following Figures, is for illustration purposes only and not for limitation.

FIG. 1 is a schematic of an HSV particle.

FIG. 2 is a schematic overview of the HSV life cycle. FIG. 2 has been modified from Ibanez, F. J., et al., “Experimental Dissection of the Lytic Replication Cycles of Herpes Simplex Virus in vitro,” Front Microbiol. 2018; 9:2406, which is incorporated herein by reference in its entirety.

FIGS. 3A-3F show expression levels in HEK293T cells transfected with nucleoside-modified RNA (modRNA) encoding HSV-2 gC (gC2), gD (gD2), or gE (gE2) antigens. Cells were transfected with 0.2 μg/mL modRNA encoding gC2 or gD2 antigen constructs, or 0.4 μg/mL modRNA encoding gE2 antigen construct, using a commercial transfection reagent (FIGS. 3A-C) or with LNP formulated RNA encoding a combination of all three antigens in a mass ratio of 1:1:1 (FIGS. 3D-F); concentrations depicted). Expression of gC2, gD2 and gE2 protein was detected by flow cytometry using primary monoclonal mouse antibodies detecting the respective antigen and a secondary fluorescent tagged anti-mouse antibody. Representative data from one experiment showing median fluorescence intensities (MFI) of the total HEK293T population for gC2 antigen constructs (FIGS. A and D), for gD2 antigen constructs (FIGS. B and E), and for gE2 antigen constructs (FIGS. C and F). Data shown are mean±SD of HEK293T transfections performed in triplicates. 1600: IL2 secretory signal and HSV-2 gC antigen. 1601: HSV-2 gD secretory signal and HSV-2 gD antigen. 1602: IL2 secretory signal and HSV-2 gE antigen. 3233: HSV-1 gD secretory signal and HSV-2 gC antigen. 3234: HSV-2 gD secretory signal and HSV-2 gD antigen. 3235: HSV-2 gD secretory signal and HSV-2 gE antigen.

FIG. 4 shows a schematic overview of a study in guinea pigs investigating a vaccine candidate against HSV-2. Guinea pigs were immunized IM on day 0 and day 28 with an HSV-2 vaccine candidate containing total gC2/gD2/gE2 RNA at a concentration of 3 μg, 15 μg, or PBS control, as outlined in Table 18. Twenty-eight days after the second immunization i.e., on day 56, animals were bled. On day 60, the guinea pigs were challenged with a lethal dose of 5×10⁵ PFU of HSV-2 strain MS (25-fold LD50). d=day; DRG=dorsal root ganglia; HSV-2=herpes simplex virus-2; gC2=glycoprotein C from herpes simplex virus-2; gD2=glycoprotein D from herpes simplex virus-2; gE2=glycoprotein E from herpes simplex virus-2; IM=intramuscular; PFU=plaque forming unit; RNA-LNP315=RNA lipid nanoparticles formulated with ALC-0315.

FIGS. 5A-5C show serum IgG antibody titers observed one month after a 2nd immunization in guinea pigs immunized with an HSV-2 vaccine candidate described herein. Serum antibody titers were determined by ELISA at day 56, 4 weeks after the second immunization with a composition (“trivalent vaccine”) comprising three polyribonucleotides encoding glycoprotein C (gC), glycoprotein D (gD) and glycoprotein E (gE), respectively. The dose level represents total RNA content of three RNAs encoding for the respective gC2, gD2 and gE2 antigens in a 1:1:1 ratio. Geometric mean±95% CI and individual animal values are shown. P values were calculated by Kruskal-Wallis test. *=p value≤0.05; **=p value≤0.01; **p value≤0.0001; gC2=glycoprotein C from herpes simplex virus-2; gD2=glycoprotein D from herpes simplex virus-2; gE2=glycoprotein E from herpes simplex virus-2; IgG=immunoglobulin G; RNA=ribonucleic acid, GMT=geometric mean; CI=confidence interval. As shown in FIG. 5, administration of the HSV-2 vaccine candidate induced high IgG antibody titers against each of gC2 (FIG. 5A), gD2 (FIG. 5B) and gE2 (FIG. 5C), with a 15 μg dose inducing higher titers for gC and gD antigens than a 3 μg dose.

FIGS. 6A-6C show vaginal IgG antibody titers in guinea pigs one month after a 2nd immunization with an HSV-2 modRNA vaccine described herein. Vaginal antibody titers were determined by ELISA at day 56, four weeks after the second immunization with a trivalent vaccine. The dose level represents total RNA content of three RNAs encoding for the respective gC2, gD2 and gE2 antigens in a 1:1:1 ratio. Geometric mean±95% CI and individual animal values are shown. P values were calculated by Kruskal-Wallis test. *=p value≤0.05; **=p value≤0.01; ***=p value≤0.001; ****=p value≤0.0001; gC2=glycoprotein C from herpes simplex virus-2; gD2=glycoprotein D from herpes simplex virus-2; gE2=glycoprotein E from herpes simplex virus-2; IgG=immunoglobulin G; RNA=ribonucleic acid; GMT=geometric mean; CI=confidence interval. As shown in FIG. 6, high vaginal IgG titers were induced against each of gC2 (FIG. 6A), gD2 (FIG. 6B), and gE2 (FIG. 6C), with a 15 μg dose inducing higher titers for gE antigen than a 3 μg dose.

FIG. 7 shows serum neutralizing antibody titers to HSV-2 in guinea pigs one month after a 2nd immunization with an HSV-2 modRNA vaccine described herein. Neutralizing antibody titers were determined using a serum HSV-2 plaque reduction assay and defined as highest dilution of serum with 5% human complement that reduced the number of HSV-2 plaques by 50%. Samples were collected at day 56, 4 weeks after the second immunization. The dose level represents total RNA content of three RNAs encoding for the respective gC2, gD2 and gE2 antigens in a 1:1:1 ratio. Geometric mean±95% CI and individual animal values are shown. P values were calculated by Mann-Whitney test. *=p-value≤0.05; gC2=glycoprotein C from herpes simplex virus-2; gD2=glycoprotein D from herpes simplex virus-2; gE2=glycoprotein E from herpes simplex virus-2; RNA=ribonucleic acid; GMT=geometric mean; CI=confidence interval. As shown in FIG. 7, high neutralization titers were observed at both a 3 μg dose and a 15 μg dose, with a 15 μg dose inducing higher neutralizing antibody titers than a 3 μg dose.

FIGS. 8A-8C show weight loss in guinea pigs administered an HSV-2 vaccine described herein, following HSV-2 viral challenge. Relative body weight changes in guinea pigs up to 14 days after viral challenge with a lethal intravaginal dose of HSV-2 at day 60, approximately one month after second immunization with PBS (FIG. 8A), 3 μg (FIG. 8B) or 15 μg (FIG. 8C) of trivalent vaccine, or PBS. The dose level represents total RNA content of three RNAs encoding for the respective gC2, gD2 and gE2 antigens in a 1:1:1 ratio. PBS=phosphate buffered saline; gC2=glycoprotein C from herpes simplex virus-2; gD2=glycoprotein D from herpes simplex virus-2; gE2=glycoprotein E from herpes simplex virus-2; RNA=ribonucleic acid. As shown in FIG. 8, administration of a 3 μg dose of an HSV-2 vaccine decreased body weight loss relative to the PBS negative control, and a 15 μg dose of an HSV-2 vaccine decreased body weight losses further still.

FIG. 9 shows survival of guinea pigs immunized with an HSV-2 vaccine described herein, up to day 48 after HSV-2 viral challenge. Probability of survival of guinea pigs up to 48 days after lethal intravaginal challenge with HSV-2 at day 60, approximately one month after second immunization with 3 μg or 15 μg of trivalent vaccine, or PBS. The dose level represents total RNA content of three RNAs encoding for the respective gC2, gD2 and gE2 antigens in a 1:1:1 ratio. P values were calculated by log-rank (Mantel-Cox) test. **=p-value≤0.01; PBS=phosphate buffered saline, gC2=glycoprotein C from herpes simplex virus-2; gD2=glycoprotein D from herpes simplex virus-2; gE2=glycoprotein E from herpes simplex virus-2; HSV-2=herpes simplex virus-2; RNA=ribonucleic acid. As shown in FIG. 9, administration of a 3 μg dose of an HSV-2 vaccine significantly increased survival of the guinea pigs, and administration a 15 μg dose increased survival further still.

FIGS. 10A-10C show individual evaluation of genital disease in guinea pigs administered an RNA composition described herein, up to day 48 after challenge with a lethal intravaginal dose of HSV-2. Results at day 60, approximately one month after the second vaccination with an HSV-2 modRNA vaccine are shown. FIG. 10A shows the mean number of days with genital disease during this period and FIG. 10B shows the mean severity of genital lesions of days with genital disease. Mean±SEM and individual animal values are shown. FIG. 10C shows the mean number of urinary retention days. The dose level represents total RNA content of three RNAs encoding for the respective gC2, gD2 and gE2 antigens in a 1:1:1 ratio. P values were calculated by Mann-Whitney test. Black circles with red outlines are associated with animals that succumbed after viral challenge in the PBS group. *=p-value≤0.05; HSV-2=herpes simplex virus-2; SEM=standard error of the mean; PBS=phosphate buffered saline, gC2=glycoprotein C from herpes simplex virus-2; gD2=glycoprotein D from herpes simplex virus-2; gE2=glycoprotein E from herpes simplex virus-2; RNA=ribonucleic acid. Because a majority of the animals in the PBS control group died within two weeks of viral challenge, disease score as measured by number of genital lesion days and/or severity of genital lesions was underrepresented in this group, and a statistical analysis of this group was not performed. As shown in FIG. 10, administering an HSV-2 vaccine described herein reduces the number of days during which a genital lesion was observed, decreased the severity of lesions that were observed, and decreased urinary retention days.

FIG. 11 shows cumulative disease score in guinea pigs administered an RNA composition described herein, up to day 48 after challenge with a lethal intravaginal dose of HSV-2. Results at day 60 are shown, approximately one month after the second vaccination. The mean number of days with genital disease per group is shown over the course of 48 days. The dose level represents total RNA content of three RNAs encoding for the respective gC2, gD2 and gE2 antigens in a 1:1:1 ratio. No scoring for days after death was assigned to animals that succumbed to viral disease. HSV-2=herpes simplex virus-2; PBS=phosphate buffered saline. gC2=glycoprotein C from herpes simplex virus-2; gD2=glycoprotein D from herpes simplex virus-2; gE2=glycoprotein E from herpes simplex virus-2; RNA=ribonucleic acid. As shown in FIG. 11, administration of 3 μg of an HSV-2 vaccine significantly decreased mean cumulative disease days, and administration 15 μg decreased mean cumulative disease days further still.

FIGS. 12A-12C show vaginal virus titers in guinea pigs administered an HSV-2 vaccine described herein, 2 and 4 days after viral challenge. Vaginal HSV-2 titers were determined by plaque assay 2 days (FIG. 12A) and 4 days (FIG. 12B) after a lethal intravaginal challenge with HSV-2. Results are plotted as means±SEM and individual animal values. Mean days of genital shedding of HSV-2 DNA were analyzed by PCR and displayed in (FIG. 12C). The dose level for the HSV-2 vaccine represents total RNA content of three RNAs in a 1:1:1 ratio encoding for the respective gC2, gD2 and gE2 antigens. P values were calculated by Kruskal-Wallis test. SEM=standard error of the mean; gC2=glycoprotein C from herpes simplex virus-2; gD2=glycoprotein D from herpes simplex virus-2; gE2=glycoprotein E from herpes simplex virus-2; HSV-2=herpes simplex virus-2; DNA=deoxyribonucleic acid; RNA=ribonucleic acid.

FIGS. 13A-13B show DNA copy numbers in DRG and spinal cord of guinea pigs administered an HSV-2 vaccine disclosed herein, on day 48 after viral challenge. DRG and spinal cord HSV-2 DNA copy numbers in guinea pigs on day 48 following viral challenge with a lethal intravaginal dose of HSV-2 were analyzed by qPCR. HSV-2 genome copies in DRG (FIG. 13A) and spinal cord (FIG. 13B) relative to GAPDH expression at day 48 after viral challenge are shown for immunized animals. Mean±SEM and individual animal values are shown. The dose level for the HSV-2 vaccine represents total RNA content of three RNAs in a 1:1:1 ratio encoding for the respective gC2, gD2 and gE2 antigens. P values were calculated by Mann-Whitney test. DRG=dorsal root ganglia; SEM=standard error of the mean; gC2=glycoprotein C from herpes simplex virus-2; gD2=glycoprotein D from herpes simplex virus-2; gE2=glycoprotein E from herpes simplex virus-2; HSV-2=herpes simplex virus-2; DNA=deoxyribonucleic acid; RNA=ribonucleic acid.

FIGS. 14A-14F show expression levels in HEK293T cells transfected with RNA encoding HSV-2 gC (gC2), gD (gD2) and gE (gE2) antigens. Cells were transfected with 0.2 μg/mL modRNA encoding gC2 and gD2 antigen constructs, and 0.4 μg/mL modRNA encoding gE2 antigen constructs, using a commercial transfection reagent. Expression of gC2, gD2 and gE2 protein was detected by flow cytometry using primary monoclonal mouse antibodies detecting the respective antigen and a secondary fluorescent tagged anti-mouse antibody. Representative data from one experiment showing median fluorescence intensities (MFI) for the total HEK293T population for gC2 antigen constructs (FIGS. 14A-C), for gD2 antigen constructs (FIGS. 14D-E) and for gE2 antigen constructs (FIG. 14F). Data shown are mean±SD of HEK293T transfections performed in triplicates. 1600: IL2 secretory signal and HSV-2 gC antigen (version 2). 1873: IL2 secretory signal and HSV-2 gC antigen (version 4). 2537: HSV-1 gD secretory signal and HSV-2 gC antigen (version 1). 2538: HSV-1 gD secretory signal and HSV-2 gC antigen (version 2). 2541: HSV-1 gB secretory signal and HSV-2 gC antigen (version 2). 2547: HSV-2 gE secretory signal and HSV-2 gC antigen (version 2). 1601: HSV-2 gD secretory signal and HSV-2 gD antigen (version 2). 1602: IL2 secretory signal and HSV-2 gE antigen (version 2). 2138: HSV-2 gE secretory signal and HSV-2 gC antigen (version 4). 2140: HSV-1 gD secretory signal and HSV-2 gC antigen (version 4). 2141: HSV-1 gB secretory signal and HSV-2 gC antigen (version 4). 2539: HSV-1 gD secretory signal and HSV-2 gC antigen (version 4). 2540: HSV-1 gB secretory signal and HSV-2 gC antigen (version 1). 2546: HSV-2 gE secretory signal and HSV-2 gC antigen (version 1). 2548: HSV-2 gE secretory signal and HSV-2 gC antigen (version 4). 2784: HSV-2 gC secretory signal and HSV-2 gC antigen (version 2). 2785: HSV-2 gC secretory signal and HSV-2 gC antigen (version 2). 1876: IL2 secretory signal and HSV-2 gC antigen (version 3). 1874: HSV-2 gD secretory signal and HSV-2 gD antigen (version 1), 1877: HSV-2 gD secretory signal and HSV-2 gD antigen (version 3), 1659: HSV-2 gD secretory signal and HSV-2 gD antigen (version 2), 1660: HSV-2 gD secretory signal and HSV-2 gE antigen (version 2), 2143: HSV-1 gD secretory signal and HSV-2 gE antigen (version 4). 1913: HSV-2 gE secretory signal and HSV-2 gE antigen (version 2). 2553: HSV-1 gD secretory signal and HSV-2 gE antigen (version 2).

FIGS. 15A-15F show transfection rates and expression levels in HEK293T cells transfected with RNA encoding HSV-2 gC (gC2), gD (gD2) and gE (gE2) antigens. Cells were transfected with 0.2 μg/mL modRNA encoding antigens using a commercial transfection reagent. Expression of gC2, gD2 and gE2 protein was detected by flow cytometry using primary monoclonal mouse antibodies detecting the respective antigen and a secondary fluorescent tagged anti-mouse antibody. Representative data from one experiment showing percentage of gC2 (FIG. 15A) and gE2 (FIG. 15B) protein-expressing cells and median fluorescence intensities of gC2, gD2 and gE2 (MFI) (FIG. 15C, FIG. 15D, and FIG. 15E, respectively) of the total HEK293T population are depicted per antigen. Data shown are mean±SD of HEK293T transfections performed in triplicates. Cumulative total HEK expression data from up to n=7 (FIG. 15F) experiments are shown in relation to the gC2 (1600, IL2 secretory signal and HSV-2 gC antigen (version 2)), gD2 (1601, HSV-2 gD secretory signal and HSV-2 gD antigen (version 2)) or gE2 (1602, IL2 secretory signal and HSV-2 gE antigen (version 2)) construct, respectively. The RNA constructs characterized in FIG. 15 were found to produce similar or improved expression as compared to 1600 (IL2 secretory signal and HSV-2 gC antigen (version 2)), 1601 (HSV-2 gD secretory signal and HSV-2 gD antigen (version 2)) and 1602 (IL2 secretory signal and HSV-2 gE antigen (version 2)). 1597: IL2 secretory signal and HSV-2 gC antigen; 1598: HSV-2 gD secretory signal and HSV-2 gD antigen; 1599: IL2 secretory signal and HSV-2 gE antigen.

FIGS. 16A-B show flow diagram of Part A (FIG. 16A) and Part B (FIG. 16B) of Example 5. DL=dose level; P=placebo (isotonic NaCl solution); V=BNT163 vaccine.

FIG. 17 describes a dose escalating schema for Part A in Example 5. Abbreviations: d=day; DL=dose level; IRC=Internal Review Committee.

FIG. 18A-D show expression levels in HEK293T cells transfected with RNA encoding HSV-2 gC (gC2), gD (gD2) or gE (gE2) antigen constructs. 1600: IL2 secretory signal and HSV-2 gC antigen. 2787: HSV-2 gD secretory signal and HSV-2 gC antigen. 2542: HSV-1 gB secretory signal and HSV-2 gC antigen. 2786: HSV-2 gC secretory signal and HSV-2 gC antigen. 1602: IL2 secretory signal and HSV-2 gE antigen. 1911: HSV-2 gD secretory signal and HSV-2 gE antigen. 2143: HSV-1 gD secretory signal and HSV-2 gE antigen. 2552: HSV-1 gD secretory signal and HSV-2 gE antigen. 2554: HSV-1 gD secretory signal and HSV-2 gE antigen. 2788: HSV-2 gE secretory signal and HSV-2 gE antigen. 2790: HSV-2 gE secretory signal and HSV-2 gE antigen. 2791: HSV-2 gE secretory signal and HSV-2 gE antigen. 2792: HSV-2 gE secretory signal and HSV-2 gE antigen.

FIG. 19A-C show secretion levels in HEK293T cells transfected with RNA encoding HSV-2 gC (gC2), gD (gD2) or gE (gE2) antigen constructs. 1600: IL2 secretory signal and HSV-2 gC antigen. 1873: IL2 secretory signal and HSV-2 gC antigen. 1876: IL2 secretory signal and HSV-2 gC antigen. 2138: HSV-2 gE secretory signal and HSV-2 gC antigen. 2140: HSV-1 gD secretory signal and HSV-2 gC antigen. 2140: HSV-1 gD secretory signal and HSV-2 gC antigen. 2141: HSV-1 gB secretory signal and HSV-2 gC antigen. 2537: HSV-1 gD secretory signal and HSV-2 gC antigen. 2538: HSV-1 gD secretory signal and HSV-2 gC antigen. 2539: HSV-1 gD secretory signal and HSV-2 gC antigen. 2540: HSV-1 gB secretory signal and HSV-2 gC antigen. 2541: HSV-1 gB secretory signal and HSV-2 gC antigen. 2542: HSV-1 gB secretory signal and HSV-2 gC antigen. 2546: HSV-2 gE secretory signal and HSV-2 gC antigen. 2547: HSV-2 gE secretory signal and HSV-2 gC antigen. 2548: HSV-2 gE secretory signal and HSV-2 gC antigen. 2784: HSV-2 gC secretory signal and HSV-2 gC antigen. 2785: HSV-2 gC secretory signal and HSV-2 gC antigen. 2786: HSV-2 gC secretory signal and HSV-2 gC antigen. 2787: HSV-2 gD secretory signal and HSV-2 gC antigen. 3233: HSV-1 gD secretory signal and HSV-2 gC antigen. 1601: HSV-2 gD secretory signal and HSV-2 gD antigen. 1659: HSV-2 gD secretory signal and HSV-2 gD antigen. 3234: HSV-2 gD secretory signal and HSV-2 gD antigen. 1602: IL2 secretory signal and HSV-2 gE antigen. 1911: HSV-2 gD secretory signal and HSV-2 gE antigen. 1660: HSV-2 gD secretory signal and HSV-2 gE antigen. 1913: HSV-2 gE secretory signal and HSV-2 gE antigen. 2143: HSV-1 gD secretory signal and HSV-2 gE antigen. 2552: HSV-1 gD secretory signal and HSV-2 gE antigen. 2553: HSV-1 gD secretory signal and HSV-2 gE antigen. 2554: HSV-1 gD secretory signal and HSV-2 gE antigen. 2788: HSV-2 gE secretory signal and HSV-2 gE antigen. 2790: gE2 secretory signal and a gE2 antigen. 2791: gE2 secretory signal and HSV-2 gE antigen. 2792: gE2 secretory signal and a gE2 antigen. 3235: gD2 secretory signal and HSV-2 gE antigen.

### CERTAIN DEFINITIONS

In general, terminology used herein is in accordance with its understood meaning in the art, unless clearly indicated otherwise. Explicit definitions of certain terms are provided below; meanings of these and other terms in particular instances throughout this specification will be clear to those skilled in the art from context.

In order that the present invention may be more readily understood, certain terms are first defined below. Additional definitions for the following terms and other terms are set forth throughout the specification.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Agent: As used herein, the term “agent”, may refer to a physical entity or phenomenon. In some embodiments, an agent may be characterized by a particular feature and/or effect. In some embodiments, an agent may be a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that comprises a polymer. In some embodiments, the term may refer to a compound or entity that comprises one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Amino acid: In its broadest sense, as used herein, the term “amino acid” refers to a compound and/or substance that can be, is, or has been incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Antibody agent: As used herein, the term “antibody agent” refers to an agent that specifically binds to a particular antigen. In some embodiments, the term encompasses a polypeptide or polypeptide complex that includes immunoglobulin structural elements sufficient to confer specific binding. For example, in some embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent in or comprises a polypeptide whose amino acid sequence includes structural elements recognized by those skilled in the art to correspond to CDRs1, 2, and 3 of an antibody variable domain; in some such embodiments, an antibody agent in or comprises a polypeptide or set of polypeptides whose amino acid sequence(s) together include structural elements recognized by those skilled in the art to correspond to both heavy chain and light chain variable region CDRs, e.g., heavy chain CDRs 1, 2, and/or 3 and light chain CDRs 1, 2, and/or 3. In some embodiments, an antibody agent is a polypeptide protein having a binding domain which is homologous or largely homologous to an immunoglobulin-binding domain. In some embodiments, an antibody agent may be or comprise a polyclonal antibody preparation. In some embodiments, an antibody agent may be or comprise a monoclonal antibody preparation. In some embodiments, an antibody agent may include one or more constant region sequences that are characteristic of a particular organism, such as a camel, human, mouse, primate, rabbit, rat; in many embodiments, an antibody agent may include one or more constant region sequences that are characteristic of a human. In some embodiments, an antibody agent may include one or more sequence elements that would be recognized by one skilled in the art as a humanized sequence, a primatized sequence, a chimeric sequence, etc. In some embodiments, an antibody agent may be a canonical antibody (e.g., may comprise two heavy chains and two light chains). In some embodiments, an antibody agent may be in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′) 2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, etc.), or other pendant group (e.g., poly-ethylene glycol, etc.)).

Antigen: Those skilled in the art, reading the present specification, will appreciate that the term “antigen” refers to a molecule that is recognized by the immune system, e.g., in particular embodiments the adaptive immune system, such that it elicits an antigen-specific immune response. In some embodiments, an antigen-specific immune response may be or comprise generation of antibodies and/or antigen-specific T cells. In some embodiments, an antigen is a peptide or polypeptide that comprises at least one epitope against which an immune response can be generated. In one embodiment, an antigen is presented by cells of the immune system such as antigen presenting cells like dendritic cells or macrophages. In one embodiments, an antigen or a processed product thereof such as a T-cell epitope is bound by a T- or B-cell receptor, or by an immunoglobulin molecule such as an antibody. Accordingly, an antigen or a processed product thereof may react specifically with antibodies or T lymphocytes (T cells). In one embodiment, an antigen is a parasitic antigen. In accordance with the present disclosure, in some embodiments, an antigen may be delivered by RNA molecules as described herein. In some embodiments, a peptide or polypeptide antigen can be 2-100 amino acids, including for example, 5 amino acids, 10 amino acids, 15 amino acids, 20 amino acids, 25 amino acids, 30 amino acids, 35 amino acids, 40 amino acids, 45 amino acids, or 50 amino acids in length. In some embodiments, a peptide or polypeptide antigen can be greater than 50 amino acids. In some embodiments, a peptide or polypeptide antigen can be greater than 100 amino acids. In some embodiments, an antigen is recognized by an immune effector cell. In some embodiments, an antigen if recognized by an immune effector cell is able to induce in the presence of appropriate co-stimulatory signals, stimulation, priming and/or expansion of the immune effector cell carrying an antigen receptor recognizing the antigen. In the context of the embodiments of the present disclosure, in some embodiments, an antigen can be presented or present on the surface of a cell, e.g., an antigen presenting cell. In one embodiment, an antigen is presented by a diseased cell such as a virus-infected cell. In one embodiment, an antigen receptor is a TCR which binds to an epitope of an antigen presented in the context of MHC. In one embodiment, binding of a TCR when expressed by T cells and/or present on T cells to an antigen presented by cells such as antigen presenting cells results in stimulation, priming and/or expansion of said T cells. In one embodiment, binding of a TCR when expressed by T cells and/or present on T cells to an antigen presented on diseased cells results in cytolysis and/or apoptosis of the diseased cells, wherein said T cells preferably release cytotoxic factors, e.g., perforins and granzymes.

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of, susceptibility to, severity of, stage of, etc the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Binding: Those skilled in the art, reading the present specification, will appreciate that the term “binding” typically refers to a non-covalent association between or among entities or moieties. In some embodiments, binding data are expressed in terms of “IC50”. As is understood in the art, IC50 is the concentration of an assessed agent in a binding assay at which 50% inhibition of binding of reference agent known to bind the relevant binding partner is observed. In some embodiments, assays are run under conditions in which the assays are run (e.g., limiting binding target and reference concentrations), these values approximate K_{D} values. Assays for determining binding are well known in the art and are described in detail, for example, in PCT publications WO 94/20127 and WO 94/03205, and other publications such Sidney et al., Current Protocols in Immunology 18.3.1 (1998); Sidney, et al., J. Immunol. 154:247 (1995); and Sette, et al., Mol. Immunol. 31:813 (1994). Alternatively, binding can be expressed relative to binding by a reference standard peptide. For example, can be based on its IC50, relative to the IC50 of a reference standard peptide. Binding can also be determined using other assay systems including those using: live cells (e.g., Ceppellini et al., Nature 339:392 (1989); Christnick et al., Nature 352:67 (1991); Busch et al., Int. Immunol. 2:443 (1990); Hill et al., J. Immunol. 147:189 (1991); del Guercio et al., J. Immunol. 154:685 (1995)), cell free systems using detergent lysates (e.g., Cerundolo et al., J. Immunol 21:2069 (1991)), immobilized purified MHC (e.g., Hill et al., J. Immunol. 152, 2890 (1994); Marshall et al., J. Immunol. 152:4946 (1994)), ELISA systems (e.g., Reay et al., EMBO J. 11:2829 (1992)), surface plasmon resonance (e.g., Khilko et al., J. Biol. Chem. 268:15425 (1993)); high flux soluble phase assays (Hammer et al., J. Exp. Med. 180:2353 (1994)), and measurement of class I MHC stabilization or assembly (e.g., Ljunggren et al., Nature 346:476 (1990); Schumacher et al., Cell 62:563 (1990); Townsend et al., Cell 62:285 (1990); Parker et al., J. Immunol. 149:1896 (1992)).

Cap: As used herein, the term “cap” refers to a structure comprising or essentially consisting of a nucleoside-5 ‘-triphosphate that is typically joined to a 5’-end of an uncapped RNA (e.g., an uncapped RNA having a 5′-diphosphate). In some embodiments, a cap is or comprises a guanine nucleotide. In some embodiments, a cap is or comprises a naturally-occurring RNA 5′ cap, including, e.g., but not limited to a 7-methylguanosine cap, which has a structure designated as “m7G.” In some embodiments, a cap is or comprises a synthetic cap analog that resembles an RNA cap structure and possesses the ability to stabilize RNA if attached thereto, including, e.g., but not limited to anti-reverse cap analogs (ARCAs) known in the art). Those skilled in the art will appreciate that methods for joining a cap to a 5′ end of an RNA are known in the art. For example, in some embodiments, a capped RNA may be obtained by in vitro capping of RNA that has a 5′ triphosphate group or RNA that has a 5′ diphosphate group with a capping enzyme system (including, e.g., but not limited to vaccinia capping enzyme system or Saccharomyces cerevisiae capping enzyme system). Alternatively, a capped RNA can be obtained by in vitro transcription (IVT) of a single-stranded DNA template in the presence of a dinucleotide or trinucleotide cap analog.

Cell-mediated immunity: “Cell-mediated immunity,” “cellular immunity,” “cellular immune response,” or similar terms are meant to include a cellular response directed to cells characterized by expression of an antigen, in particular characterized by presentation of an antigen with class I or class II MHC. A cellular response relates to immune effector cells, in particular to T cells or T lymphocytes which act as either “helpers” or “killers.” The helper T cells (also termed CD4⁺ T cells or CD4 T cells) play a central role by regulating the immune response and the killer cells (also termed cytotoxic T cells, cytolytic T cells, CD8⁺ T cells, CD8 T cells, or CTLs) kill diseased cells such as virus-infected cells, preventing the production of more diseased cells.

Co-administration: As used herein, the term “co-administration” refers to use of a pharmaceutical composition (e.g., immunogenic composition, e.g., vaccine) described herein and an additional therapeutic agent. The combined use of a pharmaceutical composition (e.g., immunogenic composition, e.g., vaccine) described herein and an additional therapeutic agent may be performed concurrently or separately (e.g., sequentially in any order). In some embodiments, a pharmaceutical composition (e.g., immunogenic composition, e.g., vaccine) described herein and an additional therapeutic agent may be combined in one pharmaceutically-acceptable carrier, or they may be placed in separate carriers and delivered to a target cell or administered to a subject at different times. Each of these situations is contemplated as falling within the meaning of “co-administration” or “combination,” provided that a pharmaceutical composition (e.g., immunogenic composition, e.g., vaccine) described herein and an additional therapeutic agent are delivered or administered sufficiently close in time that there is at least some temporal overlap in biological effect(s) generated by each on a target cell or a subject being treated.

Codon-optimized: As used herein, the term “codon-optimized” refers to alteration of codons in a coding region of a nucleic acid molecule to reflect the typical codon usage of a host organism without preferably altering the amino acid sequence encoded by the nucleic acid molecule. Within the context of the present disclosure, in some embodiments coding regions are codon-optimized for optimal expression in a subject to be treated using the RNA molecules described herein. In some embodiments, codon-optimization may be performed such that codons for which frequently occurring tRNAs are available are inserted in place of “rare codons.” In some embodiments, codon-optimization may include increasing guanosine/cytosine (G/C) content of a coding region of RNA described herein as compared to the G/C content of the corresponding coding sequence of a wild type RNA, wherein the amino acid sequence encoded by the RNA is preferably not modified compared to the amino acid sequence.

Combination therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Corresponding to: As used herein, the term “corresponding to” refers to a relationship between two or more entities. For example, the term “corresponding to” may be used to designate the position/identity of a structural element in a compound or composition relative to another compound or composition (e.g., to an appropriate reference compound or composition). For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/Hhsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure. Those of skill in the art will also appreciate that, in some instances, the term “corresponding to” may be used to describe an event or entity that shares a relevant similarity with another event or entity (e.g., an appropriate reference event or entity). To give but one example, a gene or protein in one organism may be described as “corresponding to” a gene or protein from another organism in order to indicate, in some embodiments, that it plays an analogous role or performs an analogous function and/or that it shows a particular degree of sequence identity or homology, or shares a particular characteristic sequence element.

Derived: In the context of an amino acid sequence (peptide or polypeptide) “derived from” a designated amino acid sequence (peptide or polypeptide), it refers to a structural analogue of a designated amino acid sequence. In some embodiments, an amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

Designed: As used herein, the term “designed” refers to an agent (i) whose structure is or was selected by the hand of man; (ii) that is produced by a process requiring the hand of man; and/or (iii) that is distinct from natural substances and other known agents.

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Encode: As used herein, the term “encode” or “encoding” refers to sequence information of a first molecule that guides production of a second molecule having a defined sequence of nucleotides (e.g., RNA) or a defined sequence of amino acids. For example, a DNA molecule can encode an RNA molecule (e.g., by a transcription process that includes a DNA-dependent RNA polymerase enzyme). An RNA molecule can encode a polypeptide (e.g., by a translation process). Thus, a gene, a cDNA, or an RNA molecule (e.g., an RNA) encodes a polypeptide if transcription and translation of RNA corresponding to that gene produces the polypeptide in a cell or other biological system. In some embodiments, a coding region of an RNA molecule encoding a target antigen refers to a coding strand, the nucleotide sequence of which is identical to the RNA sequence of such a target antigen. In some embodiments, a coding region of an RNA molecule encoding a target antigen refers to a non-coding strand of such a target antigen, which may be used as a template for transcription of a gene or cDNA.

Engineered: In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature.

Epitope: As used herein, the term “epitope” refers to a moiety that is specifically recognized by an immunoglobulin (e.g., antibody or receptor) binding component. For example, an epitope may be recognized by a T cell, a B cell, or an antibody. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized). Accordingly, in some embodiments, an epitope of an antigen may include a continuous or discontinuous portion of the antigen. In some embodiments, an epitope is or comprises a T cell epitope. In some embodiments, an epitope may have a length of about 5 to about 30 amino acids, or about 10 to about 25 amino acids, or about 5 to about 15 amino acids, or about 5 to 12 amino acids, or about 6 to about 9 amino acids.

Expression: As used herein, the term “expression” of a nucleic acid sequence refers to the generation of a gene product from the nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, etc); (3) translation of an RNA into a polypeptide or protein; and/or (4) post-translational modification of a polypeptide or protein.

Five prime untranslated region: As used herein, the terms “five prime untranslated region” or “5′ UTR” refer to a sequence of an RNA molecule between a transcription start site and a start codon of a coding region of an RNA. In some embodiments, “5′ UTR” refers to a sequence of an RNA molecule that begins at a transcription start site and ends one nucleotide (nt) before a start codon (usually AUG) of a coding region of an RNA molecule, e.g., in its natural context.

Fragment: The term “fragment” as used herein in the context of a nucleic acid sequence (e.g., RNA sequence) or an amino acid sequence may typically be a portion of a reference sequence. In some embodiments, a reference sequence is a full-length sequence of e.g., a nucleic acid sequence or an amino acid sequence. Accordingly, a fragment, typically, refers to a sequence that is identical to a corresponding stretch within a reference sequence. In some embodiments, a fragment comprises a continuous stretch of nucleotides or amino acid residues that corresponds to at least 10%, 20%, 30%, 40%, 50%, 60%, 70%, 80%, 90%, 95% of the total length of a reference sequence from which the fragment is derived. In some embodiments, the term “fragment”, with reference to an amino acid sequence (peptide or polypeptide), relates to a part of an amino acid sequence, e.g., a sequence which represents the amino acid sequence shortened at the N-terminus and/or C-terminus. In some embodiments, a fragment of an amino acid sequence comprises at least 6, in particular at least 8, at least 12, at least 15, at least 20, at least 30, at least 50, or at least 100 consecutive amino acids from an amino acid sequence.

Homology: As used herein, the term “homology” or “homolog” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or polypeptide molecules are considered to be “homologous” to one another if their sequences are at least 15%, 20%, 25%, 30%, 35%, 40%, 45%, 50%, 55%, 60%, 65%, 70%, 75%, 80%, 85%, 90%, 95%, or 99% identical. In some embodiments, polynucleotide molecules (e.g., DNA molecules and/or RNA molecules) and/or polypeptide molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution.

Humoral immunity: As used herein, the term “humoral immunity” or “humoral immune response” refers to antibody production and the accessory processes that accompany it, including: Th2 activation and cytokine production, germinal center formation and isotype switching, affinity maturation and memory cell generation. It also refers to the effector functions of antibodies, which include pathogen neutralization, classical complement activation, and opsonin promotion of phagocytosis and pathogen elimination.

Identity: As used herein, the term “identity” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or between polypeptide molecules are considered to be “substantially identical” to one another if their sequences are at least 25%, 30%, 35%, 40%, 45%, 50%, 55%, 60%, 65%, 70%, 75%, 80%, 85%, 90%, 95%, 96%, 97%, 98%, or 99% identical. Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 75%, at least 80%, at least 85%, at least 90%, at least 95%, at least 96%, at least 97%, at least 98%, at least 99%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller, 1989, which has been incorporated into the ALIGN program (version 2.0). In some embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Increased, Induced, or Reduced: As used herein, these terms or grammatically comparable comparative terms, indicate values that are relative to a comparable reference measurement. For example, in some embodiments, an assessed value achieved with a provided pharmaceutical composition (e.g., immunogenic composition, e.g., vaccine) may be “increased” relative to that obtained with a comparable reference pharmaceutical composition (e.g., immunogenic composition, e.g., vaccine). Alternatively or additionally, in some embodiments, an assessed value achieved in a subject may be “increased” relative to that obtained in the same subject under different conditions (e.g., prior to or after an event; or presence or absence of an event such as administration of a pharmaceutical composition (e.g., immunogenic composition, e.g., vaccine) as described herein, or in a different, comparable subject (e.g., in a comparable subject that differs from the subject of interest in prior exposure to a condition, e.g., absence of administration of a pharmaceutical composition (e.g., immunogenic composition, e.g., vaccine) as described herein.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance. In some embodiments, the term “reduced” or equivalent terms refers to a reduction in the level of an assessed value by at least 5%, at least 10%, at least 20%, at least 50%, at least 75% or higher, as compared to a comparable reference. In some embodiments, the term “reduced” or equivalent terms refers to a complete or essentially complete inhibition, i.e., a reduction to zero or essentially to zero. In some embodiments, the term “increased” or “induced” refers to an increase in the level of an assessed value by at least 10%, at least 20%, at least 30%, at least 40%, at least 50%, at least 80%, at least 100%, at least 200%, at least 500%, or higher, as compared to a comparable reference.

Ionizable: The term “ionizable” refers to a compound or group or atom that is charged at a certain pH. In the context of an ionizable amino lipid, such a lipid or a function group or atom thereof bears a positive charge at a certain pH. In some embodiments, an ionizable amino lipid is positively charged at an acidic pH. In some embodiments, an ionizable amino lipid is predominately neutral at physiological pH values, e.g., in some embodiments about 7.0-7.4, but becomes positively charged at lower pH values. In some embodiments, an ionizable amino lipid may have a pKa within a range of about 5 to about 7.

Isolated: The term “isolated” means altered or removed from the natural state. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell.

Lipid: As used herein, the terms “lipid” and “lipid-like material” are broadly defined as molecules which comprise one or more hydrophobic moieties or groups and optionally also one or more hydrophilic moieties or groups. Molecules comprising hydrophobic moieties and hydrophilic moieties are also typically denoted as amphiphiles.

RNA lipid nanoparticle: As used herein, the term “RNA lipid nanoparticle” refers to a nanoparticle comprising at least one lipid and RNA molecule(s). In some embodiments, an RNA lipid nanoparticle comprises at least one ionizable amino lipid. In some embodiments, an RNA lipid nanoparticle comprises at least one ionizable amino lipid, at least one helper lipid, and at least one polymer-conjugated lipid (e.g., PEG-conjugated lipid). In various embodiments, RNA lipid nanoparticles as described herein can have an average size (e.g., Z-average) of about 100 nm to 1000 nm, or about 200 nm to 900 nm, or about 200 nm to 800 nm, or about 250 nm to about 700 nm. In some embodiments of the present disclosure, RNA lipid nanoparticles can have a particle size (e.g., Z-average) of about 30 nm to about 200 nm, or about 30 nm to about 150 nm, about 40 nm to about 150 nm, about 50 nm to about 150 nm, about 60 nm to about 130 nm, about 70 nm to about 110 nm, about 70 nm to about 100 nm, about 80 nm to about 100 nm, about 90 nm to about 100 nm, about 70 to about 90 nm, about 80 nm to about 90 nm, or about 70 nm to about 80 nm. In some embodiments, an average size of lipid nanoparticles is determined by measuring the particle diameter. In some embodiments, RNA lipid nanoparticles may be prepared by mixing lipids with RNA molecules described herein.

Lipidoid: As used herein, a “lipidoid” refers to a lipid-like molecule. In some embodiments, a lipoid is an amphiphilic molecule with one or more lipid-like physical properties. In the context of the present disclosure, the term lipid is considered to encompass lipidoids.

Nanoparticle: As used herein, the term “nanoparticle” refers to a particle having an average size suitable for parenteral administration. In some embodiments, a nanoparticle has a longest dimension (e.g., a diameter) of less than 1,000 nanometers (nm). In some embodiments, a nanoparticle may be characterized by a longest dimension (e.g., a diameter) of less than 300 nm. In some embodiments, a nanoparticle may be characterized by a longest dimension (e.g., a diameter) of less than 100 nm. In many embodiments, a nanoparticle may be characterized by a longest dimension between about 1 nm and about 100 nm, or between about 1 μm and about 500 nm, or between about 1 nm and 1,000 nm. In many embodiments, a population of nanoparticles is characterized by an average size (e.g., longest dimension) that is below about 1,000 nm, about 500 nm, about 100 nm, about 50 nm, about 40 nm, about 30 nm, about 20 nm, or about 10 nm and often above about 1 nm. In many embodiments, a nanoparticle may be substantially spherical so that its longest dimension may be its diameter. In some embodiments, a nanoparticle has a diameter of less than 100 nm as defined by the National Institutes of Health.

Naturally occurring: The term “naturally occurring” as used herein refers to an entity that can be found in nature. For example, a peptide or nucleic acid that is present in an organism (including viruses) and can be isolated from a source in nature and which has not been intentionally modified by man in the laboratory is naturally occurring.

Neutralization: As used herein, the term “neutralization” refers to an event in which binding agents such as antibodies bind to a biological active site of a virus such as a receptor binding protein, thereby inhibiting the parasitic infection of cells. In some embodiments, the term “neutralization” refers to an event in which binding agents eliminate or significantly reduce ability of infecting cells.

Nucleic acid particle: A “nucleic acid particle” can be used to deliver nucleic acid to a target site of interest (e.g., cell, tissue, organ, and the like). A nucleic acid particle may comprise at least one cationic or cationically ionizable lipid or lipid-like material, at least one cationic polymer such as protamine, or a mixture thereof and nucleic acid. In some embodiments, a nucleic acid particle is a lipid nanoparticle. In some embodiments, a nucleic acid particle is a lipoplex particle.

Nucleic acid/Polynucleotide: As used herein, the term “nucleic acid” refers to a polymer of at least 10 nucleotides or more. In some embodiments, a nucleic acid is or comprises DNA. In some embodiments, a nucleic acid is or comprises RNA. In some embodiments, a nucleic acid is or comprises peptide nucleic acid (PNA). In some embodiments, a nucleic acid is or comprises a single stranded nucleic acid. In some embodiments, a nucleic acid is or comprises a double-stranded nucleic acid. In some embodiments, a nucleic acid comprises both single and double-stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 6-O-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or more residues or nucleotides long.

Nucleotide: As used herein, the term “nucleotide” refers to its art-recognized meaning. When a number of nucleotides is used as an indication of size, e.g., of a polynucleotide, a certain number of nucleotides refers to the number of nucleotides on a single strand, e.g., of a polynucleotide.

Patient: As used herein, the term “patient” refers to any organism who is suffering or at risk of a disease or disorder or condition. Typical patients include animals (e.g., mammals such as mice, rats, rabbits, non-human primates, and/or humans). In some embodiments, a patient is a human. In some embodiments, a patient is suffering from or susceptible to one or more diseases or disorders or conditions. In some embodiments, a patient displays one or more symptoms of a disease or disorder or condition. In some embodiments, a patient has been diagnosed with one or more diseases or disorders or conditions. In some embodiments, a disease or disorder or condition that is amenable to provided technologies is or includes an HSV infection. In some embodiments, a patient is receiving or has received certain therapy to diagnose and/or to treat a disease, disorder, or condition. In some embodiments, a patient is a patient suffering from or susceptible to an HSV infection.

PEG-conjugated lipid: The term “PEG-conjugated lipid” refers to a molecule comprising a lipid portion and a polyethylene glycol portion.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for parenteral administration, for example, by subcutaneous, intramuscular, or intravenous injection as, for example, a sterile solution or suspension formulation.

Pharmaceutically effective amount: The term “pharmaceutically effective amount” or “therapeutically effective amount” refers to the amount which achieves a desired reaction or a desired effect alone or together with further doses. In the case of the treatment of a particular disease, a desired reaction in some embodiments relates to inhibition of the course of the disease. In some embodiments, such inhibition may comprise slowing down the progress of a disease and/or interrupting or reversing the progress of the disease. In some embodiments, a desired reaction in a treatment of a disease may be or comprise delay or prevention of the onset of a disease or a condition. An effective amount of pharmaceutical compositions (e.g., immunogenic compositions, e.g., vaccines) described herein will depend, for example, on a disease or condition to be treated, the severity of such a disease or condition, individual parameters of the patient, including, e.g., age, physiological condition, size and weight, the duration of treatment, the type of an accompanying therapy (if present), the specific route of administration and similar factors. Accordingly, doses of pharmaceutical compositions (e.g., immunogenic compositions, e.g., vaccines) described herein may depend on various of such parameters. In the case that a reaction in a patient is insufficient with an initial dose, higher doses (or effectively higher doses achieved by a different, more localized route of administration) may be used.

Poly(A) sequence: As used herein, the term “poly(A) sequence” or “poly-A tail” refers to an uninterrupted or interrupted sequence of adenylate residues which is typically located at the 3′-end of an RNA molecule. Poly(A) sequences are known to those of skill in the art and may follow the 3′-UTR in the RNAs described herein. An uninterrupted poly(A) sequence is characterized by consecutive adenylate residues. In nature, an uninterrupted poly(A) sequence is typical. RNAs disclosed herein can have a poly(A) sequence attached to the free 3′-end of the RNA by a template-independent RNA polymerase after transcription or a poly(A) sequence encoded by DNA and transcribed by a template-dependent RNA polymerase.

Polypeptide: As used herein, the term “polypeptide” refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications comprise acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide.

Prevent: As used herein, the term “prevent” or “prevention” when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Recombinant: The term “recombinant” in the context of the present disclosure means “made through genetic engineering”. In some embodiments, a “recombinant” entity such as a recombinant nucleic acid in the context of the present disclosure is not naturally occurring.

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Ribonucleic acid (RNA): As used herein, the term “RNA” or “polyribonucleotide” refers to a polymer of ribonucleotides. In some embodiments, an RNA is single stranded. In some embodiments, an RNA is double stranded. In some embodiments, an RNA comprises both single and double stranded portions. In some embodiments, an RNA can comprise a backbone structure as described in the definition of “Nucleic acid/Polynucleotide” above. An RNA can be a regulatory RNA (e.g., siRNA, microRNA, etc.), or a messenger RNA (mRNA). In some embodiments where an RNA is a mRNA. In some embodiments where an RNA is a mRNA, an RNA typically comprises at its 3′ end a poly(A) region. In some embodiments where an RNA is a mRNA, an RNA typically comprises at its 5′ end an art-recognized cap structure, e.g., for recognizing and attachment of a mRNA to a ribosome to initiate translation. In some embodiments, an RNA is a synthetic RNA. Synthetic RNAs include RNAs that are synthesized in vitro (e.g., by enzymatic synthesis methods and/or by chemical synthesis methods).

Ribonucleotide: As used herein, the term “ribonucleotide” encompasses unmodified ribonucleotides and modified ribonucleotides. For example, unmodified ribonucleotides include the purine bases adenine (A) and guanine (G), and the pyrimidine bases cytosine (C) and uracil (U). Modified ribonucleotides may include one or more modifications including, but not limited to, for example, (a) end modifications, e.g., 5′ end modifications (e.g., phosphorylation, dephosphorylation, conjugation, inverted linkages, etc.), 3′ end modifications (e.g., conjugation, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages. The term “ribonucleotide” also encompasses ribonucleotide triphosphates including modified and non-modified ribonucleotide triphosphates.

Risk: As will be understood from context, “risk” of a disease, disorder, and/or condition refers to a likelihood that a particular individual will develop the disease, disorder, and/or condition. In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from individuals comparable to a particular individual. In some embodiments, relative risk is 0, 1, 2, 3, 4, 5, 6, 7, 8, 9, 10, or more. In some embodiments, risk may reflect one or more genetic attributes, e.g., which may predispose an individual toward development (or not) of a particular disease, disorder and/or condition. In some embodiments, risk may reflect one or more epigenetic events or attributes and/or one or more lifestyle or environmental events or attributes.

RNA lipoplex particle: As used herein, the term “RNA lipoplex particle” refers to a complex comprising liposomes, in particular cationic liposomes, and RNA molecules. Without wishing to bound by a particular theory, electrostatic interactions between positively charged liposomes and negatively charged RNA results in complexation and spontaneous formation of RNA lipoplex particles. In some embodiments, positively charged liposomes may comprise a cationic lipid, such as in some embodiments DOTMA, and additional lipids, such as in some embodiments DOPE. In one embodiment, an RNA lipoplex particle is a nanoparticle.

Selective or specific: The term “selective” or “specific”, when used herein in reference to an agent having an activity, is understood by those skilled in the art to mean that the agent discriminates between potential target entities, states, or cells. For example, in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of a target-binding moiety for one or more other potential target entities (e.g., competitors). In some embodiments, specificity is evaluated relative to that of a reference specific binding moiety. In some embodiments, specificity is evaluated relative to that of a reference non-specific binding moiety.

Stable: As used herein, the term “stable” in the context of the present disclosure refers to a pharmaceutical composition (e.g., immunogenic composition, e.g., vaccine) as a whole and/or components thereof meeting or exceeding pre-determined acceptance criteria. For example, in some embodiments, a stable pharmaceutical composition (e.g., immunogenic composition, e.g., vaccine) exhibits no unacceptable levels of microbial growth, and substantially no or no breakdown or degradation of the active biological molecule component(s). In some embodiments, a stable pharmaceutical composition (e.g., immunogenic composition, e.g., vaccine) refers to the integrity of RNA molecules being maintained at least above 90% or more. In some embodiments, a stable pharmaceutical composition (e.g., immunogenic composition, e.g., vaccine) refers to at least 90% or more (including, e.g., at least 95%, at least 96%, at least 97%, or more) of RNA molecules being maintained to be encapsulated within lipid nanoparticles. In some embodiments, a stable pharmaceutical composition (e.g., immunogenic composition, e.g., vaccine) refers to a formulation that remains capable of eliciting a desired immunologic response when administered to a subject. In some embodiments, a pharmaceutical composition (e.g., immunogenic composition, e.g., vaccine) remains stable for a specified period of time under certain conditions.

Subject: As used herein, the term “subject” refers to an organism to be administered with a composition described herein, e.g., for experimental, diagnostic, prophylactic, and/or therapeutic purposes. Typical subjects include animals (e.g., mammals such as mice, rats, rabbits, non-human primates, domestic pets, etc.) and humans. In some embodiments, a subject is a human subject. In some embodiments, a subject is suffering from a disease, disorder, or condition (e.g., an HSV infection). In some embodiments, a subject is susceptible to a disease, disorder, or condition (e.g., an HSV infection). In some embodiments, a subject displays one or more symptoms or characteristics of a disease, disorder, or condition (e.g., an HSV infection). In some embodiments, a subject displays one or more non-specific symptoms of a disease, disorder, or condition (e.g., an HSV infection). In some embodiments, a subject does not display any symptom or characteristic of a disease, disorder, or condition (e.g., an HSV infection). In some embodiments, a subject is someone with one or more features characteristic of susceptibility to or risk of a disease, disorder, or condition (e.g., an HSV infection). In some embodiments, a subject is a patient. In some embodiments, a subject is an individual to whom diagnosis and/or therapy is and/or has been administered.

Suffering from: An individual who is “suffering from” a disease, disorder, and/or condition has been diagnosed with and/or displays one or more symptoms of a disease, disorder, and/or condition.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Synthetic: As used herein, the term “synthetic” refers to an entity that is artificial, or that is made with human intervention, or that results from synthesis rather than naturally occurring. For example, in some embodiments, a synthetic nucleic acid or polynucleotide refers to a nucleic acid molecule that is chemically synthesized, e.g., in some embodiments by solid-phase synthesis. In some embodiments, the term “synthetic” refers to an entity that is made outside of biological cells. For example, in some embodiments, a synthetic nucleic acid or polynucleotide refers to a nucleic acid molecule (e.g., an RNA) that is produced by in vitro transcription using a template.

Therapy: The term “therapy” refers to an administration or delivery of an agent or intervention that has a therapeutic effect and/or elicits a desired biological and/or pharmacological effect (e.g., has been demonstrated to be statistically likely to have such effect when administered to a relevant population). In some embodiments, a therapeutic agent or therapy is any substance that can be used to alleviate, ameliorate, relieve, inhibit, prevent, delay onset of, reduce severity of, and/or reduce incidence of one or more symptoms or features of a disease, disorder, and/or condition. In some embodiments, a therapeutic agent or therapy is a medical intervention (e.g., surgery, radiation, phototherapy) that can be performed to alleviate, relieve, inhibit, present, delay onset of, reduce severity of, and/or reduce incidence of one or more symptoms or features of a disease, disorder, and/or condition.

Three prime untranslated region: As used herein, the terms “three prime untranslated region” or “3′ UTR” refer to a sequence of an RNA molecule that begins following a stop codon of a coding region of an open reading frame sequence. In some embodiments, the 3′ UTR begins immediately after a stop codon of a coding region of an open reading frame sequence, e.g., in its natural context. In other embodiments, the 3′ UTR does not begin immediately after stop codon of the coding region of an open reading frame sequence, e.g., in its natural context.

Threshold level (e.g., acceptance criteria): As used herein, the term “threshold level” refers to a level that are used as a reference to attain information on and/or classify the results of a measurement, for example, the results of a measurement attained in an assay. For example, in some embodiments, a threshold level means a value measured in an assay that defines the dividing line between two subsets of a population (e.g., a batch that satisfy quality control criteria vs. a batch that does not satisfy quality control criteria). Thus, a value that is equal to or higher than the threshold level defines one subset of the population, and a value that is lower than the threshold level defines the other subset of the population. A threshold level can be determined based on one or more control samples or across a population of control samples. A threshold level can be determined prior to, concurrently with, or after the measurement of interest is taken. In some embodiments, a threshold level can be a range of values.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition. In some embodiments, treatment may be administered to a subject at a later-stage of disease, disorder, and/or condition.

Vaccination: As used herein, the term “vaccination” refers to the administration of a composition intended to generate an immune response, for example to a disease-associated (e.g., disease-causing) agent. In some embodiments, vaccination can be administered before, during, and/or after exposure to a disease-associated agent, and in certain embodiments, before, during, and/or shortly after exposure to the agent. In some embodiments, vaccination includes multiple administrations, appropriately spaced in time, of a vaccine composition. In some embodiments, vaccination generates an immune response to an infectious agent.

Vaccine: As used herein, the term “vaccine” refers to a composition that induces an immune response upon administration to a subject. In some embodiments, an induced immune response provides protective immunity.

Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature.

Vector: as used herein, refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In some embodiments, known techniques may be used, for example, for generation or manipulation of recombinant DNA, for oligonucleotide synthesis, and for tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (4th ed., Cold Spring Harbor Laboratory Press, Cold Spring Harbor, N.Y. (2012)), which is incorporated herein by reference for any purpose.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls. The section headings used herein are for organizational purposes only and are not to be construed as limiting the subject matter described in any way.

### DETAILED DESCRIPTION OF CERTAIN EMBODIMENTS

As discussed above, the present disclosure provides pharmaceutical compositions (e.g., immunogenic compositions, e.g., vaccines) for delivering particular herpes simplex virus (HSV) antigen constructs (e.g., HSV-1 antigen constructs, HSV-2 antigen constructs, or a combination thereof) to a subject (e.g., a patient) and related technologies (e.g., methods). In particular, the present disclosure provides HSV (e.g., HSV-1, HSV-2, or both) vaccine compositions and related technologies (e.g., methods).

The present disclosure provides for example, polyribonucleotides that encode one or more HSV antigens. In some embodiments, such a polyribonucleotide can be part of an RNA construct. In some embodiments, a polyribonucleotide or RNA construct as described herein can be part of a composition (e.g., a pharmaceutical composition, e.g., an immunogenic composition, e.g., a vaccine.

In some embodiments, technologies provided herein are directed against HSV. A description of HSV and certain exemplary features is described below.

### I. Herpes Simplex Virus (HSV)

Herpes simplex virus (HSV) belongs to the alpha subfamily of the human herpesvirus family and includes two types: HSV-1 and HSV-2. The structure of HSV-1 and HSV-2 mainly include (from inside to outside) a DNA core, capsid, tegument and envelope. Each of HSV-1 and HSV-2 have a double stranded DNA genome of about 153 kb, encoding at least 80 genes. The DNA core is enclosed by an icosapentahedral capsid composed of 162 capsomeres, 150 hexons and 12 pentons, made of six different viral proteins. The DNA is surrounded by at least 20 different viral tegument proteins that have structural and regulatory roles. Some of them participating in capsid transport to the nucleus and other organelles, viral DNA entry into the nucleus, activation of early genes transcription, suppression of cellular protein biosynthesis, and mRNA degradation. The viral envelope surrounding the tegument has at least 12 different glycoproteins (B-N) on their surface. The glycoproteins may exist as heterodimers (H/L and E/I) with most existing as monomers.

HSV-1 and HSV-2 are responsible for a number of minor, moderate and severe pathologies, including oral and genital ulceration, virally induced blindness, viral encephalitis and disseminated infection of neonates. HSV-1 and HSV-2 are usually transmitted by different routes and affect different areas of the body, but the signs and symptoms that they cause can overlap. Infections caused by HSV-1 represent one of the more widespread infections of the orofacial region and commonly causes herpes labialis, herpetic stomatitis, and keratitis. HSV-2 typically causes genital herpes and is transmitted primarily by direct sexual contact with lesions. Most genital HSV infections are caused by HSV-2, however, an increasing number of genital HSV infections have been attributed to HSV-1. Genital HSV-1 infections are typically less severe and less prone to occurrence than genital HSV-2 infections.

HSV infections are transmitted through contact with herpetic lesions, mucosal surfaces, genital secretions, or oral secretions. The average incubation period after exposure is typically 4 days, but may range between 2 and 12 days. HSV particles can infect neuronal prolongations enervating peripheral tissues and establish latency in these cells, namely in the trigeminal ganglia and dorsal root ganglia of the sacral area from where they can sporadically reactivate. Additionally, similar to other herpesviruses, HSV infections are lifelong and generally asymptomatic. Without wishing to be bound by any particular theory, it is understood that HSV particles can be shed from infected individuals independent of the occurrence of clinical manifestations.

HSV infections are rarely fatal, but are characterized by blisters that can rupture and become painful. There are few clear differences in clinical presentation based on the type of infecting virus. However, as discussed above, HSV-1 infections tend to be less severe than HSV-2 infections, and patients infected with HSV-2 generally have more outbreaks.

### Lifecycle

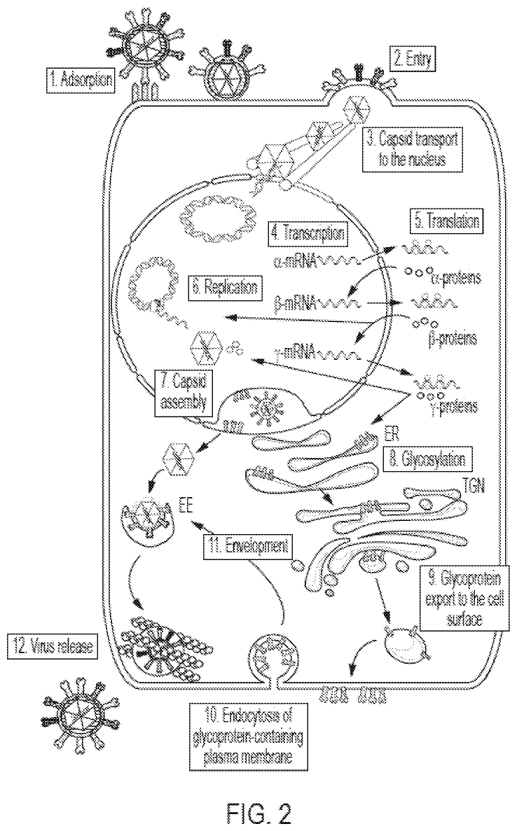

As described herein, to initiate infection, an HSV (HSV-1 or HSV-2) particle binds to the cell surface using the viral glycoproteins and fuses its envelope with the plasma membrane (see, e.g., FIG. 2, Step 1). After the fusion of membranes, the viral capsid and tegument proteins are internalized in the cytoplasm (see, e.g., FIG. 2, Step 2). Once in the cytoplasm, the viral capsid accumulates in the nucleus and releases viral DNA into the nucleus (see, e.g., FIG. 2, Step 3). HSV replicates by three rounds of transcription that yield: α (immediate early) proteins that mainly regulate viral replication; β (early) proteins that synthesise and package DNA; and γ (late) proteins, most of which are virion proteins (see, Whitley et. al., Lancet 2001 May 12; 357 (9267); Taylor et. al., Front Biosci. 2002 Mar. 1; 7: d752-64; and Ibáñez et. al., Front Microbiol. 2018 Oct. 11; 9:2406; each of which is incorporated herein by reference in its entirety) (see, e.g., FIG. 2, Steps 4-6).

The HSV capsids are assembled within the nucleus of infected cells (see, e.g., FIG. 2, Step 7). Once the assembly of viral capsids has been completed in the nucleus, these particles will continue their maturation process in this same compartment through the acquisition of tegument proteins. After leaving the nucleus, additional tegument proteins will be added to the capsids. Meanwhile, the glycoproteins are translated and glycosylated in the endoplasmic reticulum and processed in the trans-Golgi network (TGN) and then directed to multivesicular bodies (see, e.g., FIG. 2, Step 8). Then, they are exported to the plasma membrane glycoproteins within early endosomes (see, e.g., FIG. 2, Step 9). Viral capsids in the cytoplasm will then fuse with HSV-glycoprotein-containing endosomes to form infectious virions within vesicles (see, e.g., FIG. 2, Steps 10-12).

HSV (HSV-1 or HSV-2) are able to establish a latent infection. After primary infection, HSV either replicates productively in epithelial cells or enters sensory neuron axons and moves to the neuronal cell nucleus. There, the viral DNA remains as circular, extra-chromosomal DNA, and does not possess any lytic gene expression; however, latency associated transcripts are expressed and then spliced to produce mRNA. This general transcriptional silence may allow the virus to remain hidden in the cell by avoiding immune surveillance. In some aspects, provided herein are technologies (e.g., compositions and methods) for augmenting, inducing, promoting, enhancing and/or improving an immune response against HSV (e.g., HSV-1 and/or HSV-2) or a component thereof (e.g., a protein or fragment thereof). In some embodiments, technologies provided herein are designed to augment, induce, promote, enhance and/or improve immunological memory against HSV or a component thereof (e.g., a protein or fragment thereof). In some embodiments, technologies described herein are designed to act as an immunological boost to a primary vaccine, such as a vaccine directed to an epitope and/or epitopes of HSV (e.g., HSV-1 and/or HSV-2).

The virus remains in this state for the lifetime of the host, or until the proper signals reactivate the virus and new progeny are generated. Progeny virus then travel through the neuron axis to the site of the primary infection to re-initiate a lytic replication cycle.

### B. HSV Genome

The genome of HSV-1 and the genome of HSV-2 are both approximately 150 kb long of double-stranded DNA, varying slightly between subtypes and strains. The genome encodes more than 80 genes and has high GC contents: 67 and 69% for HSV-1 and HSV-2, respectively (see, Whitley et. al., Lancet 2001 May 12; 357 (9267); Taylor et. al., Front Biosci. 2002 Mar. 1; 7: d752-64; and Jiao et. al., Microbiol Resour Announc. 2019 September; 8 (39): e00993-19, which is incorporated herein by reference in its entirety).

The genome is organized as unique long region (UL) and a unique short region (US). The UL is typically bounded by terminal long (TRL) and internal long (IRL) repeats. The US is typically bounded by terminal short (IRS) and internal short (TRS) repeats. The genes found in the unique regions are present in the genome as a single copy, but genes that are encoded in the repeat regions are present in the genome in two copies (see, Whitley et. al., Lancet 2001 May 12; 357 (9267); Taylor et. al., Front Biosci. 2002 Mar. 1; 7: d752-64; and Jiao et. al., Microbiol Resour Announc. 2019 September; 8 (39): e00993-19, which is incorporated herein by reference in its entirety).

HSV contains three origins of replication within the genome that are named depending upon their location in either the Long (oriL) or Short (oriS) region of the genome. OriL is found as a single copy in the UL segment, but oriS is located in the repeat region of the Short segment; thus, it is present in the genome in two copies. Both oriL and oriS are palindromic sequences consisting of an AT-rich center region flanked by inverted repeats that contain multiple binding sites of varying affinity for the viral origin binding protein (UL9). Either oriL or one of the oriS sequences is sufficient for viral replication (see, Whitley et. al., Lancet 2001 May 12; 357 (9267); Taylor et. al., Front Biosci. 2002 Mar. 1; 7: d752-64; and Jiao et. al., Microbiol Resour Announc. 2019 September; 8 (39): e00993-19, which is incorporated herein by reference in its entirety).

The viral genome also contains signals that orchestrate proper processing of the newly synthesized genomes for packaging into pre-formed capsids. Progeny genomes are generated in long concatemers that require cleavage into unit-length monomers. For this purpose, the viral genome contains two DNA sequence elements, pac1 and pac2, that ensure proper cleavage and packaging of unit-length progeny genomes. These elements are located within the direct repeats (DR) found within the inverted repeat regions at the ends of the viral genome (see, Whitley et. al., Lancet 2001 May 12; 357 (9267); Taylor et. al., Front Biosci. 2002 Mar. 1; 7: d752-64; and Jiao et. al., Microbiol Resour Announc. 2019 September; 8 (39): e00993-19, which is incorporated herein by reference in its entirety).

### C. HSV Vaccines

Several HSV vaccines, mainly targeting HSV-2 and primarily focused on the generation of neutralizing antibodies (nAbs) targeting the viral envelope glycoprotein D as the correlate of immune protection, have been developed and evaluated in human clinical trial, see Table 1 below. Despite these vaccines exhibiting protection against HSV in preclinical studies and in some cases Phase 2 studies, none of these vaccines has demonstrated sufficient efficacy for further development or commercialization.

The present disclosure provides an insight that many prior strategies for developing pharmaceutical compositions (e.g., immunogenic compositions, e.g., vaccines) for treatment of and/or protection from HSV infection have focused primarily, or even almost exclusively, on development of neutralizing antibodies that target surface glycoproteins. The present disclosure identifies a problem with such strategies including, for example, that they may fail to appreciate value or even criticality of ensuring that an induced immune response includes significant T cell activity (in some embodiments, CD4 T cell activity, in some embodiments CD8 T cell activity, in some embodiments, both). In some embodiments, pharmaceutical compositions (e.g., immunogenic compositions, e.g., vaccines) that comprise or deliver CD4 and CD8 epitope(s) of one or more HSV antigens (e.g., HSV-1 antigens, HSV-2 antigens, or a combination thereof), e.g., in addition to one or more B cell antigens and/or epitopes may be used in treatment of and/or protection from HSV infection.

TABLE 1
Certain HSV Vaccines Under Clinical Development

| | | | Immune | |
|---|---|---|---|---|
| Name | Platform | Antigens | Response | Clinical results |
| gB/gD/MF59 | Subunit | gD and gB | Neutralzing | Phase 3 Prophylaxis |
| (Chiron) | MF59 adjuvant | | antibodies (Abs) | 9% (95% Cl: −29%, 36%) |
| gD/AS04 | Subunit | gD | Neutralizing Abs | Phase 3 Prophylaxis |
| (GSK) | AS04 adjuvant | | CD4⁺ T cells | 20% (95% CI: −29%, 50%) |
| gH-null | Single-cycle | Multiple | Neutralizing Abs | Phase 3 therapeutic |
| (Cantab | live | | | No difference in recurrences or shedding |
| Pharmaceutical) | | | | |
| HSV529 | Replication- | Multiple | Neutralizing Abs | Phase 1 |
| (Sanofi-Pasteur) | defective | | CD4⁺, CD8⁺ T | Safe but immunogenic only in doubly |
| | | | cells | seronegatives |
| Gen-003 | Subunit | gD, ICP4 | Neutralizing Abs | Phase 2 therapeutic |
| (Genocea) | Saponin | | Cytolytic T cells | Dose variable decrease shedding |
| | adjuvant | | | |
| Herp V | Heat shock | 32 HSV | Cytotoxic T cells | Phase 2 therapeutic |
| (Agenus) | protein 70-HSV | peptides | | 17% reduction in shedding frequency |
| | peptides QS-21 | (gD, additional | | |
| | adjuvant | envelope, | | |
| | | tegument, and | | |
| | | other) | | |
| Vaxfectin | DNA | gD, UL46/47 | Neutralizing Abs | Phase 1/2 therapeutic |
| (Vical) | Vaxfectin | | | Failed to reduce shedding |
| | adjuvant | | | |
| COR-1 | Codon | Codon | gD-specific Abs | Phase 2 therapeutic |
| (Admedus) | optimized DNA | optimized | Cytotoxic T cells | No difference in recurrences |
| | | gD2 and | | |
| | | ubiquitinfused | | |
| | | truncated gD2 | | |
| | | to target the | | |
| | | antigen to the | | |
| | | proteasome | | |
| ΔNLS | Deleted in ICP0 | Multiple targets | Neutralizing Abs | Phase 1 non-FDA Approved therapeutic study |
| (Rational | Replication | | gD-specific Abs | Self-reported reduction in recurrences |
| Vaccines) | competent | | | |
| | Attenuated for | | | |
| | latency | | | |

### D. Anti-Viral Treatments for HSV

The present disclosure provides the recognition that constructs and/or compositions described herein may be administered as part of regimen with other therapeutic agents. The present disclosure also recognizes that subjects that are administered constructs and/or compositions described herein may have previously been administered other therapeutic agents.

In some embodiments, for example, a subject may be receiving or had previously received an anti-viral agent for HSV. In some embodiments, an anti-viral agent can be administered to treat HSV-1 or HSV-2 infection or recurrent episodes. In some embodiments, an anti-viral agent is or comprises acyclovir, valacyclovir, famciclovir, or a combination thereof. Table 2 below provides certain information about select anti-viral agents.

TABLE 2
Antiviral Drugs for Treating HSV

| | | | |
|---|---|---|---|
| | | | Approximate price |
| Medication | Dosage | Most common adverse effects | for complete dosing |

Oral formulations:

| | | | |
|---|---|---|---|
| acyclovir | 200-400 mg 5x/day; | malaise, headache, nausea, | 35-800 mg tabs; $70 |
| | 5-365 days | vomiting | |
| valacyclovir | 1 g 3x/day; 7 days | headache, nausea, | 21-1 g tabs; $220 |
| | | abdominal pain | |
| famciclovir | 250-1500 mg | headache, nausea, fatigue, | 21-500 mg tabs; $184 |
| | 2-3x/day; 1-30 days | diarrhea | |

Intravenous formulations:

| | | |
|---|---|---|
| acyclovir | 10-15 mg/kg every 8 h; | phlebitis, acute renal failure, |
| | 7 days | nausea, vomiting, rash |
| foscarnet | 40-60 mg/kg/day, | fever, headache, renal |
| | 14-21 days | dysfunction, electrolyte |
| | | abnormalities, nausea, |
| | | vomiting diarrhea, anemia, |
| | | granulocytopenia |

Topical application

| | | |
|---|---|---|
| 1-Docosanol | Apply to affected area | Redness or swelling |
| (Treatment for | on face or lips at the | |
| HSV-1) | first sign of cold | |
| | sore/fever blister. | |

### II. Constructs

### A. Antigens

The present disclosure provides that certain HSV-2 antigens (e.g., gC, gD and/or gE antigens) and antigenic fragments thereof can be useful in preventing or treating HSV infections (e.g., HSV-2 infections, HSV-1 infections, or both). The present disclosure provides that such HSV-2 antigens antigenic portions thereof can be delivered, e.g., in HSV-2 antigen constructs and/or HSV compositions (e.g., immunogenic compositions, e.g., vaccines) as further disclosed herein.

Polyribonucleotides provided herein comprise an antigenic portion of an HSV-2 antigen (e.g., HSV-2 glycoprotein). In some embodiments, a polyribonucleotide described herein encodes an HSV-2 gC antigen or antigenic fragment thereof. In some embodiments, a polyribonucleotide described herein encodes an HSV-2 gD antigen or antigenic fragment thereof. In some embodiments, a polyribonucleotide described herein encodes an HSV-2 gE antigen or antigenic fragment thereof.

In some embodiments, a polyribonucleotide described herein encodes an antigenic portion of an HSV-2 gC antigen. In some embodiments, a polyribonucleotide described herein encodes an antigenic portion of an HSV-2 gD antigen. In some embodiments, a polyribonucleotide described herein encodes an antigenic portion of an HSV-2 gE antigen. A brief description of HSV-2 gC, gD, and gE is included below.

### Glycoprotein C (gC)

Mature HSV glycoprotein C (gC) is a 56 kDa protein that plays an important role in the initial attachment of HSV with its host target. Glycoprotein C is a type I membrane glycoprotein and is considered the primary attachment protein and principle viral ligand for binding heparin sulfate proteoglycans (HSPGs) on the cell surface of the target host. This can occur by gC interaction with HSPG rich regions found on F-actin rich membrane protrusions referred to as filopodia.

Glycoprotein C has also been shown to regulate cell entry and infection by increasing the pH threshold for acid-induced conformational changes of gB. Low pH induces reversible conformational changes to gB domains I and V, the functional region containing hydrophobic loops important in the fusion process. By positively regulating low-pH-induced conformational changes of gB, glycoprotein C enhances the ability of HSV to invade cell types, like epithelial cells, that require a low-pH mechanism for invasion.

Glycoprotein C has also been shown to play an important role in immune evasion, in addition to its role in attachment. Glycoprotein C is one of the main targets for lymphocyte cytotoxicity in certain cell types, and is able to bind complement component C3b, inhibiting complement activation. Furthermore, neutralizing epitopes that exist on other HSV glycoproteins, like gB, are protected by the presence of gC, preventing immune responses from blocking fusion.

### Glycoprotein D (gD)

Glycoprotein D is a 46 kDA type I membrane glycoprotein. The N-terminal ectodomain is comprised of 316 amino acids. Glycoprotein D is not conserved among the Herpesviridae family of viruses but is essential for HSV entry into a cell. Glycoprotein D facilitates invasion by interacting with several receptors on the cell surface, including herpesvirus entry mediator (HVEM), nectin-1 or nectin-2, and heparin sulfate that contain specific modifications. These host receptors do not function as co-receptors, as each glycoprotein interaction with host receptor occurs independently of each other. The binding of gD to one of these cellular receptors cause a conformational change that alters the auto-inhibitory closed state of gD into an active state that transmits one of two signals believed to be required for gH/gL activation. HVEM, the first gD receptor identified, belongs to the tumor necrosis factor (TNF) receptor family and is commonly found on T cells, B cells, dendritic cells, natural killer cells, macrophages, as well as non-immune cell types like neurons and epithelial cells. Within the N-terminus of Glycoprotein D, there is a 37 residue hairpin structure that forms the entire site for binding to host receptor HVEM. Specifically, residues 1-32 of the n-terminal domain of Glycoprotein D bind the cysteine-rich domain 1 (CDR1) of HVEM. When not in contact with HVEM, this N-terminal extension adopts an extended and flexible conformation.

All clinical strains of HSV-1 and HSV-2, regardless of their origin, use nectin-1 for host cell entry; however, several mutant strains of HSV-1 and HSV-2 utilize nectin-2. Furthermore, heparin sulfate is utilized by HSV-1 and not HSV-2. Glycoprotein D interaction with net-1 has been shown to be essential in some cell types such as neurons, even when other receptors to glycoproteins exist on the cell surface.

### Glycoprotein E (gE)

Glycoprotein E is approximately 53 kDa. Glycoprotein E interacts with glycoprotein I to form a heterodimeric complex that plays a key role in cell-to-cell spread and virus induced fusion. gE/gI, (unlike gB, gD, and gH/gL) are not required for fusion and entrance into a cell, but are important for cell-to-cell spread. The disruption of gE/gI complex formation has significant effects on HSV proliferation as this virus relies heavily on cell-to-cell spread for its lytic cycle. The mechanism in which gE/gI facilitate cell-to-cell spread is poorly understood, but its function is believed to be reliant on several tegument proteins. The cooperation of tegument proteins, UL11, UL16, and UL21 are believed to be important for the processing, transport, and biological activity of gE.

TABLE 3
HSV Antigen, secretory signal, and version

| | | Amino | Nucleotide | Version 1 | Version 2 | Version 3 | Version 4 |
|---|---|---|---|---|---|---|---|
| | | acid | (nt) | nt | nt | nt | nt |
| HSV-2 | Secretory | SEQ | SEQ | SEQ | SEQ | SEQ | SEQ |
| Antigen | signal | ID NO | ID NO | ID NO | ID NO | ID NO | ID NO |
| gC | IL2 | 65 | 104 | 105 | 106 | 107 | |
| | HSV-2 gD | 66 | | | | | |
| | HSV-2 gD- | 67 | 108 | | 109, 110 | | |
| | KYA | | | | | | |
| | HSV-2 gD- | 68 | | 112 | 113, 318 | 114 | |
| | KYALA | | | | | | |
| | HSV-2 gC | 131 | | | 115, 319- | | |
| | | | | | 321 | | |
| | HSV-1 gD | 159 | | 189 | 190, 348, | 191 | 192 |
| | | | | | 349 | | |
| | HSV-1 | 160 | | 193 | 194, 350 | 195 | 196 |
| | gB | | | | | | |
| | HSV-2 | 161 | | 197 | 198 | 199 | 200 |
| | gI | | | | | | |
| | HSV-2 | 162 | | 201 | 202, 351 | 203 | 204 |
| | gE + RST | | | | | | |
| | Eboz | 163 | | 205 | 206 | 207 | 208 |
| gD | IL2 | 69 | | | | | |
| | HSV-2 | 70 | 116 | 117 | 118, 119, | 120 | |
| | gD | | | | 352 | | |
| | HSV-2 gD- | 71 | | | | | |
| | KYA | | | | | | |
| | HSV-2 gD- | 72 | | | | | |
| | KYALA | | | | | | |
| gE | IL2 | 73 | 121 | 122 | 123 | 124 | |
| | HSV-2 gD | 74 | | | | | |
| | HSV-2 gD- | 75 | 125 | 126 | 127, 128, | 129 | |
| | KYA | | | | 353 | | |
| | HSV-2 gD- | 76 | | | | | |
| | KYALA | | | | | | |
| | HSV-2 gE | 132 | | | 130, 322- | | |
| | | | | | 324 | | |
| | HSV-2 gE- | 327 | | | 325 | | |
| | LVVV | | | | | | |
| | None | 328 | | | 326 | | |
| | HSV-1 | 164 | | 209 | 210 | 211 | 212 |
| | gD | | | | | | |

Example amino acid sequences of certain HSV gC, gD, and gE polypeptides are provided in Table 4 below, example deoxyribonucleic acid sequences encoding certain HSV gC, gD, and gE polypeptides are provided in Table 5, and example ribonucleic acid sequences encoding certain HSV gC, gD, and gE polypeptides are provided in Table 6 below.

TABLE 4
Example HSV Glycoprotein Amino Acid Sequences

| SEQ ID | Antigen/ | |
|---|---|---|
| NO | ORF | Amino Acid Sequence |
| 1 | HSV-2 gC | ASPGRTITVGPRGNASNAAPSASPRNASAPRTTPTPPQPRKATKSKA |
| | [UL44] | STAKPAPPPKTGPPKTSSEPVRCNRHDPLARYGSRVQIRCRFPNSTR |
| | | TEFRLQIWRYATATDAEIGTAPSLEEVMVNVSAPPGGQLVYDSAP |
| | | NRTDPHVIWAEGAGPGASPRLYSVVGPLGRQRLIIEELTLETQGMY |
| | | YWVWGRTDRPSAYGTWVRVRVFRPPSLTIHPHAVLEGQPFKATCT |
| | | AATYYPGNRAEFVWFEDGRRVFDPAQIHTQTQENPDGFSTVSTVT |
| | | SAAVGGQGPPRTFTCQLTWHRDSVSFSRRNASGTASVLPRPTITME |
| | | FTGDHAVCTAGCVPEGVTFAWFLGDDSSPAEKVAVASQTSCGRPG |
| | | TATIRSTLPVSYEQTEYICRLAGYPDGIPVLEHH |
| 260 | HSV-2 gC- | SPGRTITVGPRGNASNAAPSASPRNASAPRTTPTPPQPRKATKSKAS |
| | S | TAKPAPPPKTGPPKTSSEPVRCNRHDPLARYGSRVQIRCRFPNSTRT |
| | | EFRLQIWRYATATDAEIGTAPSLEEVMVNVSAPPGGQLVYDSAPN |
| | | RTDPHVIWAEGAGPGASPRLYSVVGPLGRQRLIIEELTLETQGMYY |
| | | WVWGRTDRPSAYGTWVRVRVFRPPSLTIHPHAVLEGQPFKATCTA |
| | | ATYYPGNRAEFVWFEDGRRVFDPAQIHTQTQENPDGFSTVSTVTS |
| | | AAVGGQGPPRTFTCQLTWHRDSVSFSRRNASGTASVLPRPTITMEF |
| | | TGDHAVCTAGCVPEGVTFAWFLGDDSSPAEKVAVASQTSCGRPGT |
| | | ATIRSTLPVSYEQTEYICRLAGYPDGIPVLEHH |
| 2 | HSV-2 gD | KYALADPSLKMADPNRFRGKNLPVLDQLTDPPGVKRVYHIQPSLE |
| | [US6] | DPFQPPSIPITVYYAVLERACRSVLLHAPSEAPQIVRGASDEARKHT |
| | | YNLTIAWYRMGDNCAIPITVMEYTECPYNKSLGVCPIRTQPRWSY |
| | | YDSFSAVSEDNLGFLMHAPAFETAGTYLRLVKINDWTEITQFILEH |
| | | RARASCKYALPLRIPPAACLTSKAYQQGVTVDSIGMLPRFIPENQR |
| | | TVALYSLKIAGWHGPKPPYTSTLLPPELSDTTNATQPELVPEDPEDS |
| | | ALLEDPAGTVSSQIPPNWHIPSIQDVAPHH |
| 3 | HSV-2 gE | RTSWKRVTSGEDVVLLPAPAGPEERTRAHKLLWAAEPLDACGPLR |
| | [US8] | PSWVALWPPRRVLETVVDAACMRAPEPLAIAYSPPFPAGDEGLYS |
| | | ELAWRDRVAVVNESLVIYGALETDSGLYTLSVVGLSDEARQVASV |
| | | VLVVEPAPVPTPTPDDYDEEDDAGVSERTPVSVPPPTPPRRPPVAPP |
| | | THPRVIPEVSHVRGVTVHMETPEAILFAPGETFGTNVSIHAIAHDDG |
| | | PYAMDVVWMRFDVPSSCAEMRIYEACLYHPQLPECLSPADAPCA |
| | | VSSWAYRLAVRSYAGCSRTTPPPRCFAEARMEPVPGLAWLASTVN |
| | | LEFQHASPQHAGLYLCVVYVDDHIHAWGHMTISTAAQYRNAVVE |
| | | QHLPQRQPEPVEPTRPHVRA |

TABLE 5
Example Deoxyribonucleic Acid Sequences for HSV Glycoproteins

| SEQ ID | Antigen/ | | |
|---|---|---|---|
| NO | ORF | Version | Polynucleotide Sequence |
| 4 | HSV-2 gC | | GCCTCCCCCGGCCGCACCATCACCGTGGGCCCCCGC |
| | [UL44] | | GGCAACGCCTCCAACGCCGCCCCCTCCGCCTCCCCC |
| | | | CGCAACGCCTCCGCCCCCCGCACCACCCCCACCCCC |
| | | | CCCCAGCCCCGCAAGGCCACCAAGTCCAAGGCCTCC |
| | | | ACCGCCAAGCCCGCCCCCCCCCCCAAGACCGGCCCC |
| | | | CCCAAGACCTCCTCCGAGCCCGTGCGCTGCAACCGC |
| | | | CACGACCCCCTGGCCCGCTACGGCTCCCGCGTGCAG |
| | | | ATCCGCTGCCGCTTCCCCAACTCCACCCGCACCGAG |
| | | | TTCCGCCTGCAGATCTGGCGCTACGCCACCGCCACC |
| | | | GACGCCGAGATCGGCACCGCCCCCTCCCTGGAGGAG |
| | | | GTGATGGTGAACGTGTCCGCCCCCCCCGGCGGCCAG |
| | | | CTGGTGTACGACTCCGCCCCCAACCGCACCGACCCC |
| | | | CACGTGATCTGGGCCGAGGGCGCCGGCCCCGGCGCC |
| | | | TCCCCCCGCCTGTACTCCGTGGTGGGCCCCCTGGGCC |
| | | | GCCAGCGCCTGATCATCGAGGAGCTGACCCTGGAGA |
| | | | CCCAGGGCATGTACTACTGGGTGTGGGGCCGCACCG |
| | | | ACCGCCCCTCCGCCTACGGCACCTGGGTGCGCGTGC |
| | | | GCGTGTTCCGCCCCCCCTCCCTGACCATCCACCCCCA |
| | | | CGCCGTGCTGGAGGGCCAGCCCTTCAAGGCCACCTG |
| | | | CACCGCCGCCACCTACTACCCCGGCAACCGCGCCGA |
| | | | GTTCGTGTGGTTCGAGGACGGCCGCCGCGTGTTCGA |
| | | | CCCCGCCCAGATCCACACCCAGACCCAGGAGAACCC |
| | | | CGACGGCTTCTCCACCGTGTCCACCGTGACCTCCGCC |
| | | | GCCGTGGGCGGCCAGGGCCCCCCCCGCACCTTCACC |
| | | | TGCCAGCTGACCTGGCACCGCGACTCCGTGTCCTTCT |
| | | | CCCGCCGCAACGCCTCCGGCACCGCCTCCGTGCTGC |
| | | | CCCGCCCCACCATCACCATGGAGTTCACCGGCGACC |
| | | | ACGCCGTGTGCACCGCCGGCTGCGTGCCCGAGGGCG |
| | | | TGACCTTCGCCTGGTTCCTGGGCGACGACTCCTCCCC |
| | | | CGCCGAGAAGGTGGCCGTGGCCTCCCAGACCTCCTG |
| | | | CGGCCGCCCCGGCACCGCCACCATCCGCTCCACCCT |
| | | | GCCCGTGTCCTACGAGCAGACCGAGTACATCTGCCG |
| | | | CCTGGCCGGCTACCCCGACGGCATCCCCGTGCTGGA |
| | | | GCACCACTAA |
| 5 | HSV-2 gC | Version 1 | AGCGCTTCTCCCGGCAGAACCATCACAGTGGGCCCT |
| | [UL44] | | AGAGGCAACGCCTCTAATGCCGCTCCTAGCGCCTCT |
| | | | CCTAGAAACGCCTCTGCTCCCAGAACCACACCTACA |
| | | | CCTCCACAGCCTAGAAAGGCCACCAAGAGCAAGGC |
| | | | CAGCACAGCCAAACCTGCTCCTCCACCTAAGACAGG |
| | | | CCCTCCAAAGACAAGCTCTGAGCCCGTGCGGTGCAA |
| | | | CAGACACGATCCACTGGCCAGATACGGCAGCCGGGT |
| | | | GCAGATCAGATGCAGATTCCCCAACAGCACCCGGAC |
| | | | CGAGTTCCGGCTCCAGATTTGGAGATACGCCACCGC |
| | | | CACAGATGCCGAGATTGGAACAGCCCCTAGCCTGGA |
| | | | AGAAGTGATGGTCAACGTTTCAGCCCCTCCTGGCGG |
| | | | CCAGCTGGTGTATGATTCTGCCCCTAACCGGACCGA |
| | | | TCCTCACGTGATATGGGCTGAAGGTGCTGGCCCAGG |
| | | | CGCAAGCCCTAGACTGTATTCTGTTGTGGGCCCTCTG |
| | | | GGCAGACAGCGGCTGATCATTGAGGAACTGACCCTG |
| | | | GAAACCCAGGGCATGTACTACTGGGTCTGGGGCAGA |
| | | | ACCGATAGACCAAGCGCCTATGGCACCTGGGTTCGA |
| | | | GTGCGAGTGTTCAGACCTCCTAGCCTGACCATCCAT |
| | | | CCTCACGCCGTTCTGGAAGGCCAGCCTTTCAAGGCC |
| | | | ACATGTACCGCCGCCACCTACTATCCCGGAAACAGA |
| | | | GCCGAGTTCGTTTGGTTCGAGGACGGCAGAAGGGTG |
| | | | TTCGACCCCGCTCAGATCCACACACAGACCCAAGAG |
| | | | AACCCCGACGGCTTTAGCACCGTGTCCACAGTGACA |
| | | | TCTGCCGCCGTTGGAGGACAGGGCCCTCCTAGAACC |
| | | | TTTACCTGCCAGCTGACCTGGCACAGAGACAGCGTG |
| | | | TCCTTCAGCAGAAGAAACGCCAGCGGCACAGCCAGC |
| | | | GTTCTGCCTAGACCTACCATCACCATGGAATTCACC |
| | | | GGCGACCACGCCGTGTGTACAGCTGGATGTGTTCCT |
| | | | GAGGGCGTGACCTTCGCTTGGTTTCTGGGCGACGAT |
| | | | AGCAGCCCTGCCGAAAAAGTGGCTGTGGCCAGCCAG |
| | | | ACAAGCTGTGGCAGACCTGGAACCGCCACCATCAGA |
| | | | AGCACACTGCCTGTCAGCTACGAGCAGACCGAGTAC |
| | | | ATCTGTCGGCTGGCCGGCTATCCTGATGGCATCCCTG |
| | | | TGCTGGAACACCACTGA |
| 261 | HSV-2 gC | Version | AGCCCTGGCAGAACCATCACAGTGGGCCCTAGAGGC |
| | [UL44] | 1.1 | AACGCCTCTAATGCCGCTCCTAGCGCCTCTCCTAGA |
| | | | AACGCCTCTGCTCCCAGAACCACACCTACACCTCCA |
| | | | CAGCCTAGAAAGGCCACCAAGAGCAAGGCCAGCAC |
| | | | AGCCAAACCTGCTCCTCCACCTAAGACAGGCCCTCC |
| | | | AAAGACAAGCTCTGAGCCCGTGCGGTGCAACAGAC |
| | | | ACGATCCACTGGCCAGATACGGCAGCCGGGTGCAGA |
| | | | TCAGATGCAGATTCCCCAACAGCACCCGGACCGAGT |
| | | | TCCGGCTCCAGATTTGGAGATACGCCACCGCCACAG |
| | | | ATGCCGAGATTGGAACAGCCCCTAGCCTGGAAGAAG |
| | | | TGATGGTCAACGTTTCAGCCCCTCCTGGCGGCCAGC |
| | | | TGGTGTATGATTCTGCCCCTAACCGGACCGATCCTCA |
| | | | CGTGATATGGGCTGAAGGTGCTGGCCCTGGCGCTTC |
| | | | CCCTAGACTGTATTCTGTTGTGGGCCCTCTGGGCAGA |
| | | | CAGCGGCTGATCATTGAGGAACTGACCCTGGAAACC |
| | | | CAGGGCATGTACTACTGGGTCTGGGGCAGAACCGAT |
| | | | AGACCAAGCGCCTATGGCACCTGGGTTCGAGTGCGA |
| | | | GTGTTCAGACCTCCTAGCCTGACCATCCATCCTCACG |
| | | | CCGTTCTGGAAGGCCAGCCTTTCAAGGCCACATGTA |
| | | | CCGCCGCCACCTACTATCCCGGAAACAGAGCCGAGT |
| | | | TCGTTTGGTTCGAGGACGGCAGAAGGGTGTTCGACC |
| | | | CCGCTCAGATCCACACACAGACCCAAGAGAACCCCG |
| | | | ACGGCTTTAGCACCGTGTCCACAGTGACATCTGCCG |
| | | | CCGTTGGAGGACAGGGCCCTCCTAGAACCTTTACCT |
| | | | GCCAGCTGACCTGGCACAGAGACAGCGTGTCCTTCA |
| | | | GCAGAAGAAACGCCAGCGGCACAGCCAGCGTTCTG |
| | | | CCTAGACCTACCATCACCATGGAATTCACCGGCGAC |
| | | | CACGCCGTGTGTACAGCTGGATGTGTTCCTGAGGGC |
| | | | GTGACCTTCGCTTGGTTTCTGGGCGACGATAGCAGC |
| | | | CCTGCCGAAAAAGTGGCTGTGGCCAGCCAGACAAGC |
| | | | TGTGGCAGACCTGGAACCGCCACCATCAGAAGCACA |
| | | | CTGCCTGTCAGCTACGAGCAGACCGAGTACATCTGT |
| | | | CGGCTGGCCGGCTATCCTGATGGCATCCCTGTGCTG |
| | | | GAACACCACTGATAA |
| 6 | HSV-2 gC | Version 2 | GCAAGCCCCGGCAGAACCATAACAGTAGGGCCACG |
| | [UL44] | | GGGGAATGCTTCCAATGCTGCACCTTCCGCTTCACC |
| | | | GAGGAATGCTTCTGCCCCAAGAACTACCCCCACTCC |
| | | | TCCTCAACCCAGGAAAGCGACAAAGTCCAAGGCCA |
| | | | GCACCGCAAAACCCGCTCCTCCTCCAAAGACTGGGC |
| | | | CCCCAAAGACAAGTAGCGAACCAGTTCGGTGCAACA |
| | | | GGCATGACCCACTTGCACGCTATGGGTCAAGAGTCC |
| | | | AGATACGGTGTCGCTTCCCTAACAGTACAAGGACTG |
| | | | AGTTTCGGCTGCAGATCTGGCGTTATGCCACAGCTA |
| | | | CTGACGCAGAGATTGGTACCGCCCCCAGTTTGGAGG |
| | | | AAGTGATGGTCAACGTGTCCGCACCCCCAGGAGGAC |
| | | | AGCTGGTCTATGACTCAGCGCCCAATAGGACCGATC |
| | | | CCCACGTGATCTGGGCAGAAGGAGCCGGTCCTGGGG |
| | | | CCTCTCCACGGCTGTACTCAGTTGTTGGCCCGCTTGG |
| | | | ACGACAGAGACTCATCATCGAGGAACTGACACTGGA |
| | | | GACACAGGGGATGTACTACTGGGTGTGGGGCCGTAC |
| | | | TGACCGCCCTTCCGCATATGGCACTTGGGTGAGAGT |
| | | | TCGCGTCTTTCGGCCCCCTTCTCTCACCATCCATCCT |
| | | | CATGCCGTGCTCGAAGGCCAGCCCTTTAAGGCCACA |
| | | | TGCACTGCTGCGACCTACTACCCTGGCAACAGAGCC |
| | | | GAGTTTGTCTGGTTTGAGGATGGTCGGCGAGTATTC |
| | | | GATCCAGCCCAGATTCACACACAAACGCAGGAAAAT |
| | | | CCGGACGGCTTCAGCACAGTGTCCACGGTGACCTCT |
| | | | GCTGCAGTTGGTGGACAAGGACCCCCTCGAACCTTC |
| | | | ACCTGTCAGCTGACCTGGCACAGAGACTCCGTAAGC |
| | | | TTCAGCCGTAGAAACGCCTCTGGAACCGCCAGTGTG |
| | | | TTGCCGAGGCCGACTATCACGATGGAATTCACAGGC |
| | | | GATCATGCCGTCTGTACTGCCGGCTGTGTGCCAGAA |
| | | | GGCGTAACCTTCGCTTGGTTTCTCGGGGATGACTCA |
| | | | AGTCCTGCAGAGAAAGTGGCTGTGGCCTCTCAGACG |
| | | | AGCTGCGGTCGACCAGGAACAGCTACCATTCGCAGC |
| | | | ACTCTGCCCGTGTCCTACGAGCAGACGGAGTACATC |
| | | | TGCAGGCTGGCCGGCTATCCCGATGGGATTCCAGTC |
| | | | CTGGAGCACCACTGA |
| 144 | HSV-2 gC | Version | GCAAGCCCCGGCAGAACCATAACAGTAGGGCCACG |
| | [UL44] | 2.1 | GGGGAATGCTTCCAATGCTGCACCTTCCGCTTCACC |
| | | | GAGGAATGCTTCTGCCCCAAGAACTACCCCCACTCC |
| | | | TCCTCAACCCAGGAAAGCGACAAAGTCCAAGGCCA |
| | | | GCACCGCAAAACCCGCTCCTCCTCCAAAGACTGGGC |
| | | | CCCCAAAGACAAGTAGCGAACCAGTTCGGTGCAACA |
| | | | GGCATGACCCACTTGCACGCTATGGGTCAAGAGTCC |
| | | | AGATACGGTGTCGCTTCCCTAACAGTACAAGGACTG |
| | | | AGTTTCGGCTGCAGATCTGGCGTTATGCCACAGCTA |
| | | | CTGACGCAGAGATTGGTACCGCCCCCAGTTTGGAAG |
| | | | AGGTGATGGTCAACGTGTCCGCACCCCCAGGAGGAC |
| | | | AGCTGGTCTATGACTCAGCGCCCAATAGGACCGATC |
| | | | CCCACGTGATCTGGGCAGAAGGAGCCGGTCCTGGGG |
| | | | CCTCTCCACGGCTGTACTCAGTTGTTGGCCCGCTTGG |
| | | | ACGACAGAGACTCATCATCGAAGAGCTGACACTGGA |
| | | | GACACAGGGGATGTACTACTGGGTGTGGGGCCGTAC |
| | | | TGACCGCCCTTCCGCATATGGCACTTGGGTGAGAGT |
| | | | TCGCGTCTTTCGGCCCCCTTCTCTCACCATCCATCCT |
| | | | CATGCCGTGCTCGAAGGCCAGCCCTTTAAGGCCACA |
| | | | TGCACTGCTGCGACCTACTACCCTGGCAACAGAGCC |
| | | | GAGTTTGTCTGGTTTGAGGATGGTCGGCGAGTATTC |
| | | | GATCCAGCCCAGATTCACACACAAACGCAGGAAAAT |
| | | | CCGGACGGCTTCAGCACAGTGTCCACGGTGACCTCT |
| | | | GCTGCAGTTGGTGGACAAGGACCCCCTCGAACCTTC |
| | | | ACCTGTCAGCTGACCTGGCACAGAGACTCCGTAAGC |
| | | | TTCAGCCGTAGAAACGCCTCTGGAACCGCCAGTGTG |
| | | | TTGCCGAGGCCGACTATCACGATGGAATTCACAGGC |
| | | | GATCATGCCGTCTGTACTGCCGGCTGTGTGCCAGAA |
| | | | GGCGTAACCTTCGCTTGGTTTCTCGGGGATGACTCA |
| | | | AGTCCTGCAGAGAAAGTGGCTGTGGCCTCTCAGACG |
| | | | AGCTGCGGTCGACCAGGAACAGCTACCATTCGCAGC |
| | | | ACTCTGCCCGTGTCCTACGAGCAGACGGAGTACATC |
| | | | TGCAGGCTGGCCGGCTATCCCGATGGGATTCCAGTC |
| | | | CTGGAGCACCACTGA |
| 262 | HSV-2 gC | Version | AGCCCCGGCAGAACCATAACAGTAGGGCCACGGGG |
| | [UL44] | 2.2 | GAATGCTTCCAATGCTGCACCTTCCGCTTCACCGAG |
| | | | GAATGCTTCTGCCCCAAGAACTACCCCCACTCCTCCT |
| | | | CAACCCAGGAAAGCGACAAAGTCCAAGGCCAGCAC |
| | | | CGCAAAACCCGCTCCTCCTCCAAAGACTGGGCCCCC |
| | | | AAAGACAAGTAGCGAACCAGTTCGGTGCAACAGGC |
| | | | ATGACCCACTTGCACGCTATGGGTCAAGAGTCCAGA |
| | | | TACGGTGTCGCTTCCCTAACAGTACAAGGACTGAGT |
| | | | TTCGGCTGCAGATCTGGCGTTATGCCACAGCTACTG |
| | | | ACGCAGAGATTGGTACCGCCCCCAGTTTGGAAGAGG |
| | | | TGATGGTCAACGTGTCCGCACCCCCAGGAGGACAGC |
| | | | TGGTCTATGACTCAGCGCCCAATAGGACCGATCCCC |
| | | | ACGTGATCTGGGCAGAAGGAGCCGGTCCTGGGGCCT |
| | | | CTCCACGGCTGTACTCAGTTGTTGGCCCGCTTGGACG |
| | | | ACAGAGACTCATCATCGAAGAGCTGACACTGGAGAC |
| | | | ACAGGGGATGTACTACTGGGTGTGGGGCCGTACTGA |
| | | | CCGCCCTTCCGCATATGGCACTTGGGTGAGAGTTCG |
| | | | CGTCTTTCGGCCCCCTTCTCTCACCATCCATCCTCAT |
| | | | GCCGTGCTCGAAGGCCAGCCCTTTAAGGCCACATGC |
| | | | ACTGCTGCGACCTACTACCCTGGCAACAGAGCCGAG |
| | | | TTTGTCTGGTTTGAGGATGGTCGGCGAGTATTCGATC |
| | | | CAGCCCAGATTCACACACAAACGCAGGAAAATCCG |
| | | | GACGGCTTCAGCACAGTGTCCACGGTGACCTCTGCT |
| | | | GCAGTTGGTGGACAAGGACCCCCTCGAACCTTCACC |
| | | | TGTCAGCTGACCTGGCACAGAGACTCCGTAAGCTTC |
| | | | AGCCGTAGAAACGCCTCTGGAACCGCCAGTGTGTTG |
| | | | CCGAGGCCGACTATCACGATGGAATTCACAGGCGAT |
| | | | CATGCCGTCTGTACTGCCGGCTGTGTGCCAGAAGGC |
| | | | GTAACCTTCGCTTGGTTTCTCGGGGATGACTCAAGTC |
| | | | CTGCAGAGAAAGTGGCTGTGGCCTCTCAGACGAGCT |
| | | | GCGGTCGACCAGGAACAGCTACCATTCGCAGCACTC |
| | | | TGCCCGTGTCCTACGAGCAGACGGAGTACATCTGCA |
| | | | GGCTGGCCGGCTATCCCGATGGGATTCCAGTCCTGG |
| | | | AGCACCACTGATAA |
| 288 | HSV-2 gC | Version | AGCCCCGGCAGAACCATAACAGTAGGGCCACGGGG |
| | [UL44] | 2.3 | GAATGCTTCCAATGCTGCACCTTCCGCTTCACCGAG |
| | | | GAATGCTTCTGCCCCAAGAACTACCCCCACTCCTCCT |
| | | | CAACCCAGGAAAGCGACAAAGTCCAAGGCCAGCAC |
| | | | CGCAAAACCCGCTCCTCCTCCAAAGACTGGGCCCCC |
| | | | AAAGACAAGTAGCGAACCAGTTCGGTGCAACAGGC |
| | | | ATGACCCACTTGCACGCTATGGGTCAAGAGTCCAGA |
| | | | TACGGTGTCGCTTCCCTAACAGTACAAGGACTGAGT |
| | | | TTCGGCTGCAGATCTGGCGTTATGCCACAGCTACTG |
| | | | ACGCAGAGATTGGTACCGCCCCCAGTTTGGAGGAAG |
| | | | TGATGGTCAACGTGTCCGCACCCCCAGGAGGACAGC |
| | | | TGGTCTATGACTCAGCGCCCAATAGGACAGACCCAC |
| | | | ATGTTATCTGGGCAGAAGGAGCCGGTCCTGGGGCCT |
| | | | CTCCACGGCTGTACTCAGTTGTTGGCCCGCTTGGACG |
| | | | ACAGAGACTCATCATCGAGGAACTGACACTGGAGAC |
| | | | ACAGGGGATGTACTACTGGGTGTGGGGCCGTACTGA |
| | | | CCGCCCTTCCGCATATGGCACTTGGGTGAGAGTTCG |
| | | | CGTCTTTCGGCCCCCTTCTCTCACCATCCATCCTCAT |
| | | | GCCGTGCTCGAAGGCCAGCCCTTTAAGGCCACATGC |
| | | | ACTGCTGCGACCTACTACCCTGGCAACAGAGCCGAG |
| | | | TTTGTCTGGTTTGAGGATGGTCGGCGAGTATTCGATC |
| | | | CAGCCCAGATTCACACACAAACGCAGGAAAATCCG |
| | | | GACGGCTTCAGCACAGTGTCCACGGTGACCTCTGCT |
| | | | GCAGTTGGTGGACAAGGACCCCCTCGAACCTTCACC |
| | | | TGTCAGCTGACCTGGCACAGAGACTCCGTAAGCTTC |
| | | | AGCCGTAGAAACGCCTCTGGAACCGCCAGTGTGTTG |
| | | | CCGAGGCCGACTATCACGATGGAATTCACAGGCGAT |
| | | | CATGCCGTCTGTACTGCCGGCTGTGTGCCAGAAGGC |
| | | | GTAACCTTCGCTTGGTTTCTCGGGGATGACTCAAGTC |
| | | | CTGCAGAGAAAGTGGCTGTGGCCTCTCAGACGAGCT |
| | | | GCGGTCGACCAGGAACAGCTACCATTCGCAGCACTC |
| | | | TGCCCGTGTCCTACGAGCAGACGGAGTACATCTGCA |
| | | | GGCTGGCCGGCTATCCCGATGGGATTCCAGTCCTGG |
| | | | AGCACCACTGA |
| 289 | HSV-2 gC | Version | AGCCCCGGCAGAACCATAACAGTAGGGCCACGGGG |
| | [UL44] | 2.4 | GAATGCTTCCAATGCTGCACCTTCCGCTTCACCGAG |
| | | | GAATGCTTCTGCCCCAAGAACTACCCCCACTCCTCCT |
| | | | CAACCCAGGAAAGCGACAAAGTCCAAGGCCAGCAC |
| | | | CGCAAAACCCGCTCCTCCTCCAAAGACTGGGCCCCC |
| | | | AAAGACAAGTAGCGAACCAGTTCGGTGCAACAGGC |
| | | | ATGACCCACTTGCACGCTATGGGTCAAGAGTCCAGA |
| | | | TACGGTGTCGCTTCCCTAACAGTACAAGGACTGAGT |
| | | | TTCGGCTGCAGATCTGGCGTTATGCCACAGCTACTG |
| | | | ACGCAGAGATTGGTACCGCCCCCAGTTTGGAGGAAG |
| | | | TGATGGTCAACGTGTCCGCACCCCCAGGAGGACAGC |
| | | | TGGTCTATGACTCAGCGCCCAATAGGACCGATCCCC |
| | | | ACGTGATCTGGGCAGAAGGAGCCGGTCCTGGGGCCT |
| | | | CTCCACGGCTGTACTCAGTTGTTGGCCCGCTTGGACG |
| | | | ACAGAGACTCATCATCGAGGAACTGACACTGGAGAC |
| | | | ACAGGGGATGTACTACTGGGTGTGGGGCCGTACTGA |
| | | | CCGCCCTTCCGCATATGGCACTTGGGTGAGAGTTCG |
| | | | CGTCTTTCGGCCCCCTTCTCTCACCATCCATCCTCAT |
| | | | GCCGTGCTCGAAGGCCAGCCCTTTAAGGCCACATGC |
| | | | ACTGCTGCGACCTACTACCCTGGCAACAGAGCCGAG |
| | | | TTTGTCTGGTTTGAGGATGGTCGGCGAGTATTCGATC |
| | | | CAGCCCAGATTCACACACAAACGCAGGAAAATCCG |
| | | | GACGGCTTCAGCACAGTGTCCACGGTGACCTCTGCT |
| | | | GCAGTTGGTGGACAAGGACCCCCTCGAACCTTCACC |
| | | | TGTCAGCTGACCTGGCACAGAGACTCCGTAAGCTTC |
| | | | AGCCGTAGAAACGCCTCTGGAACCGCCAGTGTGTTG |
| | | | CCGAGGCCGACTATCACGATGGAATTCACAGGCGAT |
| | | | CATGCCGTCTGTACTGCCGGCTGTGTGCCAGAAGGC |
| | | | GTAACCTTCGCTTGGTTTCTCGGGGATGACTCAAGTC |
| | | | CTGCAGAGAAAGTGGCTGTGGCCTCTCAGACGAGCT |
| | | | GCGGTCGACCAGGAACAGCTACCATTCGCAGCACTC |
| | | | TGCCCGTGTCCTACGAGCAGACGGAGTACATCTGCA |
| | | | GGCTGGCCGGCTATCCCGATGGGATTCCAGTCCTGG |
| | | | AGCACCACTGA |
| 330 | HSV-2 gC | Version | AGCCCCGGCAGAACCATAACAGTAGGGCCACGGGG |
| | [UL44] | 2.5 | GAATGCTTCCAATGCTGCACCTTCCGCTTCACCGAG |
| | | | GAATGCTTCTGCCCCAAGAACTACCCCCACTCCTCCT |
| | | | CAACCCAGGAAAGCGACAAAGTCCAAGGCCAGCAC |
| | | | CGCAAAACCCGCTCCTCCTCCAAAGACTGGGCCCCC |
| | | | AAAGACAAGTAGCGAACCAGTTCGGTGCAACAGGC |
| | | | ATGACCCACTTGCACGCTATGGGTCAAGAGTCCAGA |
| | | | TACGGTGTCGCTTCCCTAACAGTACAAGGACTGAGT |
| | | | TTCGGCTGCAGATCTGGCGTTATGCCACAGCTACTG |
| | | | ACGCAGAGATTGGTACCGCCCCCAGTTTGGAGGAAG |
| | | | TGATGGTCAACGTGTCCGCACCCCCAGGAGGACAGC |
| | | | TGGTCTATGACTCAGCGCCCAATAGGACCGATCCCC |
| | | | ACGTGATCTGGGCAGAAGGAGCCGGTCCTGGGGCCT |
| | | | CTCCACGGCTGTACTCAGTTGTTGGCCCGCTTGGACG |
| | | | ACAGAGACTCATCATCGAGGAACTGACACTGGAGAC |
| | | | ACAGGGGATGTACTACTGGGTGTGGGGCCGTACTGA |
| | | | CCGCCCTTCCGCATATGGCACTTGGGTGAGAGTTCG |
| | | | CGTCTTTCGGCCCCCTTCTCTCACCATCCATCCTCAT |
| | | | GCCGTGCTCGAAGGCCAGCCCTTTAAGGCCACATGC |
| | | | ACTGCTGCGACCTACTACCCTGGCAACAGAGCCGAG |
| | | | TTTGTCTGGTTTGAGGATGGTCGGCGAGTATTCGATC |
| | | | CAGCCCAGATTCACACACAAACGCAGGAAAATCCG |
| | | | GACGGCTTCAGCACAGTGTCCACGGTGACCTCTGCT |
| | | | GCAGTTGGTGGACAAGGACCCCCTCGAACCTTCACC |
| | | | TGTCAGCTGACCTGGCACAGAGACTCCGTAAGCTTC |
| | | | AGCCGTAGAAACGCCTCTGGAACCGCCAGTGTGTTG |
| | | | CCGAGGCCGACTATCACGATGGAATTCACAGGCGAT |
| | | | CATGCCGTCTGTACTGCCGGCTGTGTGCCAGAAGGC |
| | | | GTAACCTTCGCTTGGTTTCTCGGGGATGACTCAAGTC |
| | | | CTGCAGAGAAAGTGGCTGTGGCCTCTCAGACGAGCT |
| | | | GCGGTCGACCAGGAACAGCTACCATTCGCAGCACTC |
| | | | TGCCCGTGTCCTACGAGCAGACGGAGTACATCTGCA |
| | | | GGCTGGCCGGCTATCCCGATGGGATTCCAGTCCTGG |
| | | | AGCACCACTGATAA |
| 7 | HSV-2 gC | Version 3 | GCCTCCCCCGGCAGAACCATCACCGTGGGCCCCAGA |
| | [UL44] | | GGCAACGCCTCCAACGCCGCCCCCTCCGCCTCCCCC |
| | | | AGAAACGCCTCCGCCCCCAGAACCACCCCCACCCCT |
| | | | CCCCAGCCCAGAAAAGCCACCAAATCCAAAGCCTCC |
| | | | ACCGCCAAACCCGCCCCTCCTCCCAAAACCGGCCCT |
| | | | CCCAAAACCTCCTCCGAACCCGTGAGATGCAACAGA |
| | | | CACGATCCCCTGGCCAGATACGGCTCCAGAGTGCAG |
| | | | ATCAGATGCAGATTCCCCAACTCCACCAGAACCGAA |
| | | | TTCAGACTGCAGATCTGGAGATACGCCACCGCCACC |
| | | | GATGCCGAAATCGGCACCGCCCCCTCCCTGGAAGAA |
| | | | GTGATGGTGAACGTGTCCGCCCCTCCTGGCGGCCAG |
| | | | CTGGTGTACGATTCCGCCCCCAACAGAACCGATCCC |
| | | | CACGTGATCTGGGCCGAAGGCGCCGGCCCCGGCGCC |
| | | | TCCCCCAGACTGTACTCCGTGGTGGGCCCCCTGGGC |
| | | | AGACAGAGACTGATCATCGAAGAACTGACCCTGGA |
| | | | AACCCAGGGCATGTACTACTGGGTGTGGGGCAGAAC |
| | | | CGATAGACCCTCCGCCTACGGCACCTGGGTGAGAGT |
| | | | GAGAGTGTTCAGACCCCCTTCCCTGACCATCCACCC |
| | | | CCACGCCGTGCTGGAAGGCCAGCCCTTCAAAGCCAC |
| | | | CTGCACCGCCGCCACCTACTACCCCGGCAACAGAGC |
| | | | CGAATTCGTGTGGTTCGAAGATGGCAGAAGGGTGTT |
| | | | CGATCCCGCCCAGATCCACACCCAGACCCAGGAAAA |
| | | | CCCCGACGGCTTCTCCACCGTGTCCACCGTGACCTCC |
| | | | GCCGCCGTGGGCGGCCAGGGCCCTCCCAGAACCTTC |
| | | | ACCTGCCAGCTGACCTGGCACAGAGACTCCGTGTCC |
| | | | TTCTCCAGAAGAAACGCCTCCGGCACCGCCTCCGTG |
| | | | CTGCCCAGACCCACCATCACCATGGAATTCACCGGC |
| | | | GATCACGCCGTGTGCACCGCCGGCTGCGTGCCCGAA |
| | | | GGCGTGACCTTCGCCTGGTTCCTGGGCGATGATTCCT |
| | | | CCCCCGCCGAAAAAGTGGCCGTGGCCTCCCAGACCT |
| | | | CCTGCGGCAGACCCGGCACCGCCACCATCAGATCCA |
| | | | CCCTGCCCGTGTCCTACGAACAGACCGAATACATCT |
| | | | GCAGACTGGCCGGCTACCCCGATGGCATCCCCGTGC |
| | | | TGGAACACCACTGA |
| 264 | HSV-2 gC | Version | TCTCCTGGAAGAACCATCACAGTGGGACCAAGAGGA |
| | [UL44] | 3.1 | AATGCAAGCAATGCAGCACCTTCTGCTTCTCCAAGA |
| | | | AATGCTTCTGCTCCAAGAACCACCCCAACCCCTCCTC |
| | | | AGCCAAGAAAAGCAACCAAATCCAAAGCATCCACA |
| | | | GCAAAACCTGCACCTCCTCCAAAAACAGGACCTCCA |
| | | | AAAACCTCCTCTGAACCTGTGAGATGCAACAGACAT |
| | | | GATCCTCTGGCAAGATATGGATCAAGAGTGCAGATC |
| | | | AGATGCAGATTTCCAAATTCCACCAGAACAGAATTC |
| | | | AGACTCCAGATCTGGAGATATGCAACAGCAACAGAT |
| | | | GCAGAAATTGGAACAGCACCATCTCTGGAAGAAGTG |
| | | | ATGGTGAATGTGTCTGCTCCTCCTGGAGGACAGCTG |
| | | | GTGTATGATTCTGCTCCAAACAGAACAGATCCTCAT |
| | | | GTGATCTGGGCTGAAGGAGCTGGACCTGGAGCTTCT |
| | | | CCAAGACTGTACTCTGTGGTGGGACCTCTGGGAAGA |
| | | | CAGAGACTGATCATTGAAGAACTGACCCTGGAAACC |
| | | | CAGGGAATGTACTACTGGGTGTGGGGAAGAACAGA |
| | | | CAGACCTTCTGCTTATGGAACCTGGGTGAGAGTGAG |
| | | | AGTGTTCAGACCTCCTTCTCTGACCATCCACCCTCAT |
| | | | GCTGTGCTGGAAGGACAGCCTTTCAAAGCAACCTGC |
| | | | ACAGCAGCAACCTACTACCCTGGAAACAGAGCTGAA |
| | | | TTTGTGTGGTTTGAAGATGGAAGAAGGGTGTTTGAT |
| | | | CCTGCTCAGATCCACACCCAGACCCAGGAAAATCCT |
| | | | GATGGATTTTCCACAGTGTCCACAGTGACATCTGCT |
| | | | GCTGTGGGAGGACAGGGACCTCCAAGAACCTTCACC |
| | | | TGCCAGCTGACCTGGCACAGAGATTCTGTGTCTTTTT |
| | | | CAAGAAGAAATGCTTCTGGAACAGCTTCTGTGCTGC |
| | | | CAAGACCAACCATCACCATGGAATTCACAGGAGATC |
| | | | ATGCTGTGTGCACAGCTGGATGTGTGCCTGAAGGAG |
| | | | TGACCTTTGCTTGGTTTCTGGGAGATGATTCTTCTCC |
| | | | AGCTGAAAAAGTGGCTGTGGCTTCCCAGACCTCTTG |
| | | | TGGAAGACCTGGAACAGCAACCATCAGATCCACCCT |
| | | | GCCTGTGTCTTATGAACAGACAGAATACATTTGCAG |
| | | | ACTGGCTGGATACCCTGATGGAATCCCTGTGCTGGA |
| | | | ACACCACTGATAA |
| 265 | HSV-2 gC | Version 4 | TCTCCGGGACGGACTATAACCGTAGGTCCAAGAGGA |
| | [UL44] | | AACGCCTCTAACGCAGCCCCGTCTGCCTCACCACGA |
| | | | AACGCCTCAGCTCCCAGAACCACTCCTACTCCACCC |
| | | | CAGCCTAGGAAGGCGACGAAATCCAAGGCTTCCACG |
| | | | GCCAAACCCGCCCCTCCACCCAAAACCGGACCTCCT |
| | | | AAGACCAGCTCTGAACCGGTGCGGTGTAATAGGCAC |
| | | | GACCCATTGGCGCGATATGGCAGTAGGGTCCAGATA |
| | | | CGGTGCAGATTCCCAAACAGCACAAGAACAGAATTC |
| | | | CGGCTGCAAATCTGGCGATATGCAACGGCCACCGAT |
| | | | GCCGAAATCGGAACAGCACCCAGTCTGGAAGAAGT |
| | | | GATGGTGAACGTCAGTGCTCCACCTGGCGGACAACT |
| | | | GGTGTACGACTCTGCACCCAATCGCACAGATCCCCA |
| | | | CGTGATTTGGGCCGAGGGTGCTGGACCTGGGGCTTC |
| | | | ACCCAGGCTGTATAGCGTTGTAGGGCCACTTGGGAG |
| | | | GCAGAGACTCATCATTGAGGAACTGACCCTGGAAAC |
| | | | TCAGGGCATGTACTACTGGGTATGGGGCCGCACAGA |
| | | | TCGCCCCAGCGCTTATGGCACCTGGGTGCGGGTGCG |
| | | | GGTGTTTCGCCCACCCTCCCTCACCATTCACCCTCAT |
| | | | GCGGTTCTGGAGGGACAGCCTTTCAAGGCAACTTGT |
| | | | ACCGCAGCCACCTACTATCCCGGCAATAGAGCGGAG |
| | | | TTCGTCTGGTTTGAGGACGGCCGTAGGGTGTTCGAT |
| | | | CCTGCCCAGATTCACACCCAGACACAGGAGAATCCC |
| | | | GACGGCTTTAGCACAGTGAGCACTGTGACGTCTGCT |
| | | | GCCGTTGGTGGTCAAGGGCCTCCTCGTACCTTCACAT |
| | | | GCCAATTGACCTGGCACCGCGACTCAGTTAGCTTTA |
| | | | GCCGCCGGAATGCCAGTGGGACCGCCAGTGTTCTCC |
| | | | CAAGGCCGACAATCACCATGGAGTTCACTGGCGACC |
| | | | ATGCAGTGTGCACAGCTGGGTGTGTCCCAGAAGGCG |
| | | | TGACTTTCGCCTGGTTTCTGGGTGATGACTCCTCACC |
| | | | CGCCGAGAAAGTAGCTGTCGCTTCCCAGACTTCCTG |
| | | | TGGACGTCCTGGAACTGCGACAATCCGAAGCACACT |
| | | | GCCGGTTTCCTACGAGCAGACGGAGTACATATGCCG |
| | | | CCTTGCAGGCTACCCCGATGGAATTCCAGTCCTTGA |
| | | | GCACCATTGA |
| 266 | HSV-2 gC | Version | AGTCCAGGAAGGACGATTACGGTGGGACCCAGAGG |
| | [UL44] | 4.1 | TAATGCGTCCAATGCTGCGCCATCCGCTTCTCCACGG |
| | | | AACGCATCTGCACCCAGGACTACACCGACACCACCT |
| | | | CAGCCGCGCAAAGCCACCAAGAGCAAGGCCAGCAC |
| | | | AGCCAAACCCGCTCCTCCACCTAAAACCGGACCACC |
| | | | TAAGACCAGCTCTGAACCCGTCAGATGCAACAGGCA |
| | | | CGATCCGTTGGCCAGATATGGCAGTCGCGTCCAGAT |
| | | | CAGGTGTCGCTTCCCTAACAGCACACGGACCGAGTT |
| | | | CAGGCTGCAAATTTGGCGCTACGCTACAGCCACTGA |
| | | | CGCAGAGATTGGCACTGCTCCCAGTCTGGAGGAGGT |
| | | | CATGGTGAACGTGTCTGCTCCACCAGGCGGTCAGCT |
| | | | GGTCTATGACTCAGCCCCTAATCGCACAGATCCTCA |
| | | | CGTGATTTGGGCAGAAGGTGCGGGGCCTGGGGCCTC |
| | | | CCCAAGGCTCTACTCAGTGGTTGGACCCCTTGGGAG |
| | | | ACAGCGGCTGATCATCGAGGAACTGACTCTCGAAAC |
| | | | CCAAGGTATGTACTACTGGGTATGGGGCAGAACAGA |
| | | | CAGACCTTCAGCTTATGGCACCTGGGTGCGGGTGAG |
| | | | AGTGTTTAGGCCTCCCTCCCTGACGATCCATCCCCAT |
| | | | GCTGTGCTGGAAGGACAGCCGTTCAAGGCAACATGC |
| | | | ACAGCAGCCACTTACTATCCCGGAAACCGTGCTGAG |
| | | | TTTGTGTGGTTCGAGGATGGGCGACGTGTATTCGAC |
| | | | CCTGCCCAGATTCACACCCAGACACAGGAGAATCCC |
| | | | GACGGGTTTTCCACTGTGAGCACCGTGACATCAGCG |
| | | | GCAGTAGGAGGGCAGGGCCCACCCCGAACGTTCACT |
| | | | TGCCAGCTTACTTGGCATCGGGACAGTGTTAGCTTTA |
| | | | GCCGCCGGAATGCCTCTGGCACCGCATCCGTCCTTC |
| | | | CTCGCCCAACCATCACCATGGAATTCACTGGCGATC |
| | | | ACGCCGTTTGTACAGCCGGGTGTGTTCCCGAGGGAG |
| | | | TGACCTTTGCTTGGTTTCTGGGCGATGACTCAAGCCC |
| | | | AGCCGAAAAGGTGGCCGTCGCCTCCCAAACGAGCTG |
| | | | TGGGCGACCTGGCACCGCTACCATACGTAGCACTCT |
| | | | GCCCGTTTCCTACGAACAGACCGAGTATATCTGCCG |
| | | | ATTGGCCGGTTACCCCGATGGGATACCAGTCCTGGA |
| | | | GCACCACTGA |
| 267 | HSV-2 gC | Version | TCCCCGGGTCGAACAATCACTGTTGGGCCCAGGGGA |
| | [UL44] | 4.2 | AATGCCAGCAATGCTGCACCTTCAGCAAGCCCACGA |
| | | | AACGCTTCAGCACCCAGGACAACACCCACTCCACCT |
| | | | CAACCGCGGAAAGCCACCAAGAGCAAGGCAAGTAC |
| | | | CGCCAAACCCGCTCCTCCTCCCAAGACAGGGCCACC |
| | | | CAAGACCTCTAGTGAGCCAGTGAGGTGTAACCGCCA |
| | | | TGATCCCCTTGCCAGATACGGGAGCAGAGTGCAGAT |
| | | | TAGGTGCCGGTTTCCAAACTCCACGAGAACCGAATT |
| | | | TCGCCTCCAGATTTGGCGGTATGCGACTGCCACAGA |
| | | | CGCAGAGATTGGTACCGCTCCCAGCCTGGAGGAGGT |
| | | | CATGGTGAACGTGTCAGCGCCTCCGGGTGGCCAGCT |
| | | | GGTCTACGACTCTGCCCCAAATCGAACCGACCCTCA |
| | | | CGTCATCTGGGCTGAAGGAGCGGGACCAGGAGCCTC |
| | | | TCCACGCTTGTATAGCGTAGTTGGCCCTCTGGGGAG |
| | | | ACAGCGCCTGATCATTGAGGAACTGACCCTTGAGAC |
| | | | ACAGGGGATGTACTACTGGGTGTGGGGCAGGACTGA |
| | | | CAGGCCCAGTGCCTATGGAACTTGGGTTAGGGTCCG |
| | | | CGTCTTTCGGCCACCCAGTCTGACCATCCATCCACAT |
| | | | GCCGTGCTGGAAGGCCAGCCCTTCAAAGCGACTTGC |
| | | | ACTGCCGCCACGTACTATCCAGGGAATAGAGCCGAG |
| | | | TTCGTTTGGTTCGAGGATGGCCGGAGAGTATTCGAT |
| | | | CCAGCTCAGATCCACACCCAGACGCAGGAAAACCCG |
| | | | GACGGCTTTAGCACGGTGAGTACCGTCACCTCTGCT |
| | | | GCCGTCGGAGGCCAAGGACCTCCCCGTACCTTCACA |
| | | | TGCCAGCTTACATGGCACCGGGACTCAGTAAGCTTT |
| | | | TCACGTCGTAATGCATCCGGTACTGCTTCTGTGCTGC |
| | | | CTCGACCCACCATCACCATGGAGTTCACAGGGGATC |
| | | | ACGCAGTGTGTACGGCAGGCTGCGTGCCTGAAGGCG |
| | | | TGACATTCGCCTGGTTTCTCGGTGATGACTCCTCTCC |
| | | | TGCTGAAAAGGTGGCTGTAGCCTCCCAAACAAGCTG |
| | | | TGGTCGGCCTGGAACTGCCACTATACGCTCCACTCTC |
| | | | CCGGTGTCCTACGAACAGACCGAGTACATATGCAGA |
| | | | CTGGCTGGATATCCCGATGGCATTCCCGTGCTGGAG |
| | | | CATCACTGA |
| 268 | HSV-2 gC | Version | TCTCCAGGCAGAACTATCACAGTGGGACCCAGAGGG |
| | [UL44] | 4.3 | AATGCCAGCAATGCAGCCCCGAGTGCCAGCCCTCGT |
| | | | AACGCCAGCGCTCCCAGAACAACCCCAACTCCACCG |
| | | | CAGCCTAGAAAGGCGACCAAGTCCAAAGCATCCACT |
| | | | GCAAAACCAGCCCCACCTCCCAAAACGGGACCTCCC |
| | | | AAGACCAGCTCCGAGCCTGTAAGGTGCAATCGGCAT |
| | | | GACCCCTTGGCCCGATATGGCAGTCGCGTGCAGATT |
| | | | CGATGTCGGTTTCCCAACTCTACCCGGACTGAGTTCC |
| | | | GGTTGCAGATCTGGAGGTATGCGACCGCCACTGACG |
| | | | CTGAGATCGGCACAGCACCAAGCCTGGAAGAAGTG |
| | | | ATGGTGAACGTTAGTGCTCCTCCGGGCGGGCAACTC |
| | | | GTGTATGACTCCGCACCCAACCGCACAGATCCTCAC |
| | | | GTGATTTGGGCCGAAGGAGCCGGACCCGGTGCGTCA |
| | | | CCTAGGCTCTACTCTGTCGTAGGACCACTGGGCCGT |
| | | | CAACGCCTGATAATCGAGGAGCTGACTCTGGAGACA |
| | | | CAGGGTATGTACTACTGGGTCTGGGGCAGAACCGAC |
| | | | AGGCCATCTGCTTACGGGACATGGGTCCGCGTTCGA |
| | | | GTATTTCGGCCACCCTCACTGACCATACATCCCCATG |
| | | | CCGTTCTTGAAGGGCAGCCTTTCAAGGCAACCTGTA |
| | | | CTGCTGCCACATACTATCCCGGGAATAGGGCCGAGT |
| | | | TCGTCTGGTTTGAAGATGGCCGAAGGGTGTTTGACC |
| | | | CGGCTCAGATCCACACCCAGACACAGGAGAACCCCG |
| | | | ATGGCTTCAGTACGGTGTCTACCGTCACAAGCGCCG |
| | | | CTGTGGGTGGCCAAGGTCCTCCCAGAACTTTCACCT |
| | | | GTCAGCTGACGTGGCACAGGGATTCCGTGAGCTTTT |
| | | | CCCGCCGCAATGCGTCAGGGACCGCCTCCGTGCTTC |
| | | | CTCGGCCAACCATCACAATGGAATTCACGGGTGATC |
| | | | ACGCTGTCTGCACAGCTGGCTGCGTTCCTGAGGGCG |
| | | | TGACATTCGCATGGTTTCTTGGTGACGACTCATCTCC |
| | | | CGCAGAGAAGGTGGCTGTTGCCTCACAAACGAGTTG |
| | | | TGGGCGTCCAGGCACTGCCACCATTCGGTCCACCCT |
| | | | CCCCGTAAGCTACGAACAGACTGAGTATATTTGCAG |
| | | | ACTGGCTGGATACCCGGATGGGATTCCTGTCCTGGA |
| | | | ACATCACTGA |
| 269 | HSV-2 gC | Version | TCTCCCGGACGAACTATCACTGTAGGCCCAAGGGGC |
| | [UL44] | 4.4 | AACGCTAGTAATGCCGCTCCCAGTGCTTCACCACGC |
| | | | AATGCGAGCGCACCCAGAACTACACCCACACCTCCT |
| | | | CAGCCGAGGAAAGCCACCAAGTCCAAAGCCAGCAC |
| | | | CGCCAAACCCGCTCCTCCACCTAAAACAGGGCCTCC |
| | | | CAAGACCTCAAGCGAGCCCGTTAGATGCAATCGGCA |
| | | | TGATCCACTGGCTCGTTACGGTTCTCGGGTCCAGATA |
| | | | CGCTGTAGGTTTCCTAACTCCACACGAACCGAGTTC |
| | | | AGATTGCAGATCTGGAGATATGCCACAGCCACTGAC |
| | | | GCTGAGATTGGCACTGCACCTAGTCTGGAGGAGGTG |
| | | | ATGGTGAACGTGAGCGCCCCTCCTGGCGGTCAGCTT |
| | | | GTGTATGACTCAGCACCGAATCGCACAGATCCGCAC |
| | | | GTCATATGGGCCGAAGGTGCAGGGCCCGGTGCATCC |
| | | | CCTCGGCTGTATTCCGTGGTCGGACCACTCGGGCGC |
| | | | CAGAGGCTTATCATTGAGGAACTGACCCTCGAAACC |
| | | | CAGGGTATGTACTACTGGGTATGGGGCCGTACCGAC |
| | | | CGGCCTAGCGCCTACGGAACTTGGGTGAGAGTTCGG |
| | | | GTGTTCAGACCGCCAAGTCTTACAATTCACCCTCATG |
| | | | CCGTGCTCGAAGGTCAGCCATTTAAGGCCACGTGTA |
| | | | CTGCGGCTACGTACTATCCCGGGAATCGGGCTGAAT |
| | | | TCGTGTGGTTTGAGGATGGCAGGAGAGTGTTCGACC |
| | | | CAGCCCAAATCCACACCCAAACACAGGAGAACCCA |
| | | | GACGGGTTTTCCACCGTGTCAACGGTCACATCTGCC |
| | | | GCCGTCGGAGGACAAGGGCCACCCAGAACCTTCACA |
| | | | TGCCAGCTGACCTGGCATAGGGATAGCGTAAGCTTT |
| | | | AGCCGGCGAAACGCATCTGGAACGGCGAGCGTTCTG |
| | | | CCTCGACCAACAATCACCATGGAGTTCACCGGCGAT |
| | | | CACGCAGTGTGCACTGCTGGGTGTGTACCCGAAGGC |
| | | | GTGACATTTGCCTGGTTTCTGGGAGATGACTCCTCAC |
| | | | CCGCAGAAAAGGTCGCCGTTGCATCTCAGACCAGTT |
| | | | GTGGCAGGCCCGGGACTGCTACCATCCGCAGCACTC |
| | | | TGCCGGTGTCTTACGAACAGACGGAGTACATTTGCC |
| | | | GCTTGGCGGGCTATCCAGACGGCATTCCAGTTCTGG |
| | | | AGCATCACTGA |
| 8 | HSV-2 gD | | AAGTACGCCCTGGCCGACCCCTCCCTGAAGATGGCC |
| | [US6] | | GACCCCAACCGCTTCCGCGGCAAGAACCTGCCCGTG |
| | | | CTGGACCAGCTGACCGACCCCCCCGGCGTGAAGCGC |
| | | | GTGTACCACATCCAGCCCTCCCTGGAGGACCCCTTC |
| | | | CAGCCCCCCTCCATCCCCATCACCGTGTACTACGCCG |
| | | | TGCTGGAGCGCGCCTGCCGCTCCGTGCTGCTGCACG |
| | | | CCCCCTCCGAGGCCCCCCAGATCGTGCGCGGCGCCT |
| | | | CCGACGAGGCCCGCAAGCACACCTACAACCTGACCA |
| | | | TCGCCTGGTACCGCATGGGCGACAACTGCGCCATCC |
| | | | CCATCACCGTGATGGAGTACACCGAGTGCCCCTACA |
| | | | ACAAGTCCCTGGGCGTGTGCCCCATCCGCACCCAGC |
| | | | CCCGCTGGTCCTACTACGACTCCTTCTCCGCCGTGTC |
| | | | CGAGGACAACCTGGGCTTCCTGATGCACGCCCCCGC |
| | | | CTTCGAGACCGCCGGCACCTACCTGCGCCTGGTGAA |
| | | | GATCAACGACTGGACCGAGATCACCCAGTTCATCCT |
| | | | GGAGCACCGCGCCCGCGCCTCCTGCAAGTACGCCCT |
| | | | GCCCCTGCGCATCCCCCCCGCCGCCTGCCTGACCTCC |
| | | | AAGGCCTACCAGCAGGGCGTGACCGTGGACTCCATC |
| | | | GGCATGCTGCCCCGCTTCATCCCCGAGAACCAGCGC |
| | | | ACCGTGGCCCTGTACTCCCTGAAGATCGCCGGCTGG |
| | | | CACGGCCCCAAGCCCCCCTACACCTCCACCCTGCTG |
| | | | CCCCCCGAGCTGTCCGACACCACCAACGCCACCCAG |
| | | | CCCGAGCTGGTGCCCGAGGACCCCGAGGACTCCGCC |
| | | | CTGCTGGAGGACCCCGCCGGCACCGTGTCCTCCCAG |
| | | | ATCCCCCCCAACTGGCACATCCCCTCCATCCAGGAC |
| | | | GTGGCCCCCCACCACTAA |
| 9 | HSV-2 gD | Version 1 | AAATACGCCCTGGCCGATCCTAGCCTGAAGATGGCT |
| | [US6] | | GACCCCAACCGGTTCCGGGGCAAGAATCTGCCTGTT |
| | | | CTGGACCAGCTGACCGATCCTCCTGGCGTGAAACGG |
| | | | GTGTACCACATCCAGCCAAGCCTGGAAGATCCCTTC |
| | | | CAGCCTCCTAGCATCCCCATCACCGTGTACTACGCC |
| | | | GTGCTGGAAAGGGCCTGTAGAAGCGTGCTGCTGCAC |
| | | | GCCCCATCTGAAGCCCCTCAAATCGTCAGAGGCGCT |
| | | | TCCGACGAGGCCAGAAAGCACACCTACAACCTGACA |
| | | | ATCGCCTGGTACAGAATGGGCGACAACTGCGCCATT |
| | | | CCTATCACCGTGATGGAGTACACCGAGTGTCCCTAC |
| | | | AACAAGAGCCTGGGCGTGTGCCCCATCAGAACACAG |
| | | | CCTAGATGGTCCTACTACGACAGCTTCAGCGCCGTG |
| | | | TCCGAGGACAATCTGGGCTTCCTGATGCATGCCCCT |
| | | | GCCTTTGAGACAGCCGGCACCTATCTGCGGCTGGTC |
| | | | AAGATCAACGACTGGACCGAGATCACCCAGTTCATC |
| | | | CTGGAACACAGAGCCAGAGCCAGCTGCAAATACGCT |
| | | | CTGCCCCTGAGAATTCCTCCTGCCGCCTGTCTGACAA |
| | | | GCAAGGCCTATCAGCAGGGCGTGACCGTGGATAGCA |
| | | | TCGGCATGCTGCCCAGATTCATCCCCGAGAACCAGA |
| | | | GAACAGTGGCCCTGTACTCCCTGAAGATCGCCGGAT |
| | | | GGCACGGACCCAAGCCTCCATACACAAGCACACTGC |
| | | | TGCCTCCAGAGCTGAGCGACACCACCAATGCCACAC |
| | | | AGCCTGAACTGGTGCCTGAGGACCCAGAGGATTCTG |
| | | | CCCTGCTTGAAGATCCTGCCGGCACCGTGTCTAGCC |
| | | | AGATTCCTCCTAACTGGCACATCCCCAGCATCCAGG |
| | | | ATGTGGCCCCTCATCATTGA |
| 10 | HSV-2 gD | Version 2 | AAATATGCTCTCGCTGATCCGAGCCTCAAGATGGCA |
| | [US6] | | GATCCCAACCGATTTCGGGGAAAGAATCTGCCAGTA |
| | | | CTGGACCAGCTGACGGACCCACCTGGCGTCAAACGC |
| | | | GTCTACCACATACAGCCTAGTCTTGAGGACCCTTTTC |
| | | | AGCCACCGTCTATCCCCATTACCGTGTACTATGCCGT |
| | | | GCTGGAACGCGCGTGTAGGTCAGTTCTGCTGCATGC |
| | | | CCCATCCGAAGCCCCCCAGATCGTCAGAGGAGCTTC |
| | | | TGATGAAGCACGCAAACACACCTACAACCTCACAAT |
| | | | AGCGTGGTATCGAATGGGCGATAACTGCGCAATTCC |
| | | | CATCACAGTCATGGAGTACACGGAGTGCCCCTACAA |
| | | | CAAGAGCCTCGGTGTTTGCCCTATCAGGACACAACC |
| | | | CAGGTGGAGCTATTACGACAGTTTCAGCGCCGTGTC |
| | | | TGAGGACAATCTGGGGTTTCTGATGCACGCACCCGC |
| | | | CTTCGAGACTGCCGGCACCTACTTGCGGCTGGTGAA |
| | | | GATCAACGACTGGACTGAGATCACCCAGTTCATCCT |
| | | | GGAACATAGGGCCAGAGCCAGCTGCAAGTATGCTCT |
| | | | TCCCCTGCGGATTCCGCCTGCAGCATGTCTGACCTCA |
| | | | AAAGCCTACCAGCAAGGGGTGACTGTGGACAGCATT |
| | | | GGCATGCTGCCTCGTTTCATTCCCGAGAATCAACGG |
| | | | ACAGTGGCTCTGTATTCCCTGAAGATCGCAGGATGG |
| | | | CATGGGCCCAAACCACCTTATACCTCTACGTTGCTTC |
| | | | CACCAGAACTCAGTGACACCACTAATGCGACACAGC |
| | | | CAGAACTTGTGCCTGAGGATCCTGAAGATAGCGCTC |
| | | | TGTTGGAGGATCCAGCCGGTACTGTGTCCTCCCAGA |
| | | | TACCACCCAATTGGCACATTCCTTCCATTCAGGACGT |
| | | | AGCTCCGCATCACTGA |
| 145 | HSV-2 gD | Version | AAATATGCTCTCGCTGATCCGAGCCTCAAGATGGCA |
| | [US6] | 2.1 | GATCCCAACCGATTTCGGGGAAAGAATCTGCCAGTA |
| | | | CTGGACCAGCTGACGGACCCACCTGGCGTCAAACGC |
| | | | GTCTACCACATACAGCCTAGTCTTGAGGACCCTTTTC |
| | | | AGCCACCGTCTATCCCCATTACCGTGTACTATGCCGT |
| | | | GCTGGAACGCGCGTGTAGGTCAGTTCTGCTGCATGC |
| | | | CCCATCCGAAGCCCCCCAGATCGTCAGAGGAGCTTC |
| | | | TGATGAAGCACGCAAACACACCTACAACCTCACAAT |
| | | | AGCGTGGTATCGAATGGGCGATAACTGCGCAATTCC |
| | | | CATCACAGTCATGGAGTACACGGAGTGCCCCTACAA |
| | | | CAAGAGCCTCGGTGTTTGCCCTATCAGGACACAACC |
| | | | CAGGTGGAGCTATTACGACAGTTTCAGCGCCGTGTC |
| | | | TGAGGACAATCTGGGGTTTCTGATGCACGCACCCGC |
| | | | CTTCGAGACTGCCGGCACCTACTTGCGGCTGGTGAA |
| | | | GATCAACGACTGGACTGAGATCACCCAGTTCATCCT |
| | | | GGAACATAGGGCCAGAGCCAGCTGCAAGTATGCCCT |
| | | | TCCCCTGCGGATTCCGCCTGCAGCATGTCTGACCTCA |
| | | | AAAGCCTACCAGCAAGGGGTGACTGTGGACAGCATT |
| | | | GGCATGCTGCCTCGTTTCATTCCCGAGAATCAACGG |
| | | | ACAGTGGCTCTGTATTCCCTGAAGATCGCAGGATGG |
| | | | CATGGGCCCAAACCACCTTATACCTCTACGTTGCTTC |
| | | | CACCAGAACTCAGTGACACCACTAATGCGACACAGC |
| | | | CAGAACTTGTGCCTGAGGATCCTGAAGATAGCGCTC |
| | | | TGTTGGAGGATCCAGCCGGTACTGTGTCCTCCCAGA |
| | | | TACCACCCAATTGGCACATTCCTTCCATTCAGGACGT |
| | | | AGCTCCGCATCACTGA |
| 334 | HSV-2 gD | Version | AAATATGCTCTCGCTGATCCGAGCCTCAAGATGGCA |
| | [US6] | 2.2 | GATCCCAACCGATTTCGGGGAAAGAATCTGCCAGTA |
| | | | CTGGACCAGCTGACGGACCCACCTGGCGTCAAACGC |
| | | | GTCTACCACATACAGCCTAGTCTTGAGGACCCTTTTC |
| | | | AGCCACCGTCTATCCCCATTACCGTGTACTATGCCGT |
| | | | GCTGGAACGCGCGTGTAGGTCAGTTCTGCTGCATGC |
| | | | CCCATCCGAAGCCCCCCAGATCGTCAGAGGAGCTTC |
| | | | TGATGAAGCACGCAAACACACCTACAACCTCACAAT |
| | | | AGCGTGGTATCGAATGGGCGATAACTGCGCAATTCC |
| | | | CATCACAGTCATGGAGTACACGGAGTGCCCCTACAA |
| | | | CAAGAGCCTCGGTGTTTGCCCTATCAGGACACAACC |
| | | | CAGGTGGAGCTATTACGACAGTTTCAGCGCCGTGTC |
| | | | TGAGGACAATCTGGGGTTTCTGATGCACGCACCCGC |
| | | | CTTCGAGACTGCCGGCACCTACTTGCGGCTGGTGAA |
| | | | GATCAACGACTGGACTGAGATCACCCAGTTCATCCT |
| | | | GGAACATAGGGCCAGAGCCAGCTGCAAGTATGCCCT |
| | | | TCCCCTGCGGATTCCGCCTGCAGCATGTCTGACCTCA |
| | | | AAAGCCTACCAGCAAGGGGTGACTGTGGACAGCATT |
| | | | GGCATGCTGCCTCGTTTCATTCCCGAGAATCAACGG |
| | | | ACAGTGGCTCTGTATTCCCTGAAGATCGCAGGATGG |
| | | | CATGGGCCCAAACCACCTTATACCTCTACGTTGCTTC |
| | | | CACCAGAACTCAGTGACACCACTAATGCGACACAGC |
| | | | CAGAACTTGTGCCTGAGGATCCTGAAGATAGCGCTC |
| | | | TGTTGGAGGATCCAGCCGGTACTGTGTCCTCCCAGA |
| | | | TACCACCCAATTGGCACATTCCTTCCATTCAGGACGT |
| | | | AGCTCCGCATCACTGATAA |
| 11 | HSV-2 gD | Version 3 | AAATACGCCCTGGCCGATCCCTCCCTGAAAATGGCC |
| | [US6] | | GATCCCAACAGGTTCAGAGGCAAAAACCTGCCCGTG |
| | | | CTGGATCAGCTGACCGATCCCCCTGGCGTGAAAAGA |
| | | | GTGTACCACATCCAGCCCTCCCTGGAAGATCCCTTCC |
| | | | AGCCCCCTTCCATCCCCATCACCGTGTACTACGCCGT |
| | | | GCTGGAAAGAGCTTGCAGATCCGTGCTGCTGCACGC |
| | | | CCCCTCCGAAGCCCCTCAGATCGTGAGAGGCGCCTC |
| | | | CGATGAAGCCAGAAAACACACCTACAACCTGACCAT |
| | | | CGCCTGGTACAGAATGGGCGATAACTGCGCCATCCC |
| | | | CATCACCGTGATGGAATACACCGAATGCCCCTACAA |
| | | | CAAATCCCTGGGCGTGTGCCCCATCAGAACCCAGCC |
| | | | CAGATGGTCCTACTACGATTCCTTCTCCGCCGTGTCC |
| | | | GAAGATAACCTGGGCTTCCTGATGCACGCCCCCGCC |
| | | | TTCGAAACCGCCGGCACCTACCTGAGACTGGTGAAA |
| | | | ATCAACGATTGGACCGAAATCACCCAGTTCATCCTG |
| | | | GAACACAGAGCCAGAGCCTCCTGCAAATACGCCCTG |
| | | | CCCCTGAGAATCCCTCCCGCCGCCTGCCTGACCTCCA |
| | | | AAGCCTACCAGCAGGGCGTGACCGTGGATTCCATCG |
| | | | GCATGCTGCCCAGATTCATCCCCGAAAACCAGAGAA |
| | | | CCGTGGCCCTGTACTCCCTGAAAATCGCCGGCTGGC |
| | | | ACGGCCCCAAACCCCCTTACACCTCCACCCTGCTGC |
| | | | CCCCTGAACTGTCCGATACCACCAACGCCACCCAGC |
| | | | CCGAACTGGTGCCCGAAGATCCCGAAGATTCCGCCC |
| | | | TGCTGGAAGATCCCGCCGGCACCGTGTCCTCCCAGA |
| | | | TCCCTCCCAACTGGCACATCCCCTCCATCCAGGATGT |
| | | | GGCCCCTCACCACTGA |
| 12 | HSV-2 gE | | CGCACCTCCTGGAAGCGCGTGACCTCCGGCGAGGAC |
| | [US8] | | GTGGTGCTGCTGCCCGCCCCCGCCGGCCCCGAGGAG |
| | | | CGCACCCGCGCCCACAAGCTGCTGTGGGCCGCCGAG |
| | | | CCCCTGGACGCCTGCGGCCCCCTGCGCCCCTCCTGG |
| | | | GTGGCCCTGTGGCCCCCCCGCCGCGTGCTGGAGACC |
| | | | GTGGTGGACGCCGCCTGCATGCGCGCCCCCGAGCCC |
| | | | CTGGCCATCGCCTACTCCCCCCCCTTCCCCGCCGGCG |
| | | | ACGAGGGCCTGTACTCCGAGCTGGCCTGGCGCGACC |
| | | | GCGTGGCCGTGGTGAACGAGTCCCTGGTGATCTACG |
| | | | GCGCCCTGGAGACCGACTCCGGCCTGTACACCCTGT |
| | | | CCGTGGTGGGCCTGTCCGACGAGGCCCGCCAGGTGG |
| | | | CCTCCGTGGTGCTGGTGGTGGAGCCCGCCCCCGTGC |
| | | | CCACCCCCACCCCCGACGACTACGACGAGGAGGACG |
| | | | ACGCCGGCGTGTCCGAGCGCACCCCCGTGTCCGTGC |
| | | | CCCCCCCCACCCCCCCCCGCCGCCCCCCCGTGGCCCC |
| | | | CCCCACCCACCCCCGCGTGATCCCCGAGGTGTCCCA |
| | | | CGTGCGCGGCGTGACCGTGCACATGGAGACCCCCGA |
| | | | GGCCATCCTGTTCGCCCCCGGCGAGACCTTCGGCAC |
| | | | CAACGTGTCCATCCACGCCATCGCCCACGACGACGG |
| | | | CCCCTACGCCATGGACGTGGTGTGGATGCGCTTCGA |
| | | | CGTGCCCTCCTCCTGCGCCGAGATGCGCATCTACGA |
| | | | GGCCTGCCTGTACCACCCCCAGCTGCCCGAGTGCCT |
| | | | GTCCCCCGCCGACGCCCCCTGCGCCGTGTCCTCCTGG |
| | | | GCCTACCGCCTGGCCGTGCGCTCCTACGCCGGCTGC |
| | | | TCCCGCACCACCCCCCCCCCCCGCTGCTTCGCCGAG |
| | | | GCCCGCATGGAGCCCGTGCCCGGCCTGGCCTGGCTG |
| | | | GCCTCCACCGTGAACCTGGAGTTCCAGCACGCCTCC |
| | | | CCCCAGCACGCCGGCCTGTACCTGTGCGTGGTGTAC |
| | | | GTGGACGACCACATCCACGCCTGGGGCCACATGACC |
| | | | ATCTCCACCGCCGCCCAGTACCGCAACGCCGTGGTG |
| | | | GAGCAGCACCTGCCCCAGCGCCAGCCCGAGCCCGTG |
| | | | GAGCCCACCCGCCCCCACGTGCGCGCCTAA |
| 13 | HSV-2 gE | Version 1 | AGAACCAGCTGGAAAAGAGTGACCAGCGGCGAGGA |
| | [US8] | | TGTGGTGCTGCTTCCTGCTCCTGCTGGCCCCGAGGAA |
| | | | AGAACAAGAGCCCACAAACTGCTGTGGGCCGCTGA |
| | | | GCCTCTTGATGCCTGTGGACCTCTCAGACCTAGCTGG |
| | | | GTTGCACTGTGGCCACCTCGGAGAGTGCTGGAAACA |
| | | | GTGGTGGATGCCGCCTGCATGAGAGCCCCTGAACCT |
| | | | CTGGCCATTGCCTACTCTCCACCATTTCCAGCCGGCG |
| | | | ACGAGGGCCTGTATTCTGAGCTTGCTTGGAGAGACA |
| | | | GAGTGGCCGTGGTCAACGAGAGCCTGGTTATCTATG |
| | | | GCGCCCTGGAAACCGACAGCGGCCTGTACACACTGT |
| | | | CTGTCGTGGGCCTGTCTGACGAGGCTAGACAGGTGG |
| | | | CATCTGTGGTCCTGGTGGTGGAACCTGCTCCAGTGC |
| | | | CTACACCTACACCTGACGACTACGACGAGGAAGATG |
| | | | ACGCTGGCGTCAGCGAGAGAACCCCTGTTTCTGTGC |
| | | | CTCCTCCTACGCCTCCTCGTAGACCTCCTGTTGCTCC |
| | | | TCCAACACACCCCAGAGTGATCCCTGAAGTGTCTCA |
| | | | CGTGCGGGGCGTGACCGTGCACATGGAAACACCTGA |
| | | | GGCCATCCTGTTCGCCCCTGGCGAGACATTTGGCAC |
| | | | CAACGTGTCCATCCACGCTATCGCCCACGACGATGG |
| | | | CCCTTACGCCATGGATGTCGTGTGGATGAGATTCGA |
| | | | CGTGCCCAGCAGCTGTGCCGAGATGAGAATCTATGA |
| | | | GGCCTGCCTGTATCACCCTCAGCTGCCCGAATGTCTG |
| | | | AGCCCTGCTGATGCCCCTTGTGCCGTTAGCAGCTGG |
| | | | GCCTATAGACTGGCCGTGCGGTCTTATGCCGGCTGC |
| | | | TCTAGAACAACCCCTCCTCCTCGGTGTTTCGCCGAGG |
| | | | CCAGAATGGAACCTGTTCCTGGACTGGCCTGGCTGG |
| | | | CCTCCACAGTGAACCTGGAATTTCAGCACGCCTCTC |
| | | | CACAGCACGCCGGCCTGTATCTGTGTGTGGTGTACG |
| | | | TGGACGATCACATCCACGCCTGGGGCCACATGACCA |
| | | | TCTCTACAGCCGCTCAGTACCGGAACGCCGTGGTTG |
| | | | AACAGCATCTGCCTCAGAGACAGCCCGAGCCTGTGG |
| | | | AACCTACAAGACCTCATGTTCGGGCCTGA |
| 270 | HSV-2 gE | Version | AGAACCTCTTGGAAGCGCGTGACAAGCGGCGAGGA |
| | [US8] | 1.1 | TGTGGTTCTGCTTCCTGCTCCTGCCGGACCTGAGGAA |
| | | | AGAACAAGAGCCCACAAGCTGCTGTGGGCCGCTGA |
| | | | ACCTTTGGATGCCTGTGGACCTCTGAGGCCTTCTTGG |
| | | | GTTGCACTGTGGCCACCTCGGAGAGTGCTGGAAACA |
| | | | GTGGTGGATGCCGCCTGCATGAGAGCCCCTGAACCT |
| | | | CTGGCCATTGCCTACTCTCCACCTTTTCCAGCCGGCG |
| | | | ACGAGGGCCTGTATTCTGAGCTTGCTTGGAGAGACA |
| | | | GAGTGGCCGTGGTCAACGAGAGCCTGGTTATCTATG |
| | | | GCGCCCTGGAAACCGACAGCGGCCTGTACACACTGT |
| | | | CTGTCGTGGGCCTGTCTGACGAGGCTAGACAGGTGG |
| | | | CATCTGTGGTGCTGGTGGTGGAACCTGCTCCAGTGC |
| | | | CTACACCTACACCTGACGACTACGACGAGGAAGATG |
| | | | ACGCTGGCGTCAGCGAGAGAACCCCTGTTTCTGTGC |
| | | | CTCCTCCTACGCCTCCTCGTAGACCTCCTGTTGCTCC |
| | | | TCCAACACACCCCAGAGTGATCCCTGAAGTGTCTCA |
| | | | CGTGCGGGGCGTGACCGTGCACATGGAAACACCTGA |
| | | | GGCCATCCTGTTCGCCCCTGGCGAGACATTTGGCAC |
| | | | CAACGTGTCCATCCACGCTATCGCCCACGACGATGG |
| | | | CCCTTACGCCATGGATGTCGTGTGGATGAGATTCGA |
| | | | CGTGCCCAGCAGCTGCGCCGAGATGAGAATCTACGA |
| | | | GGCCTGCCTGTATCACCCTCAGCTGCCCGAATGTCTG |
| | | | AGCCCTGCTGATGCCCCTTGTGCCGTTAGCAGCTGG |
| | | | GCCTATAGACTGGCCGTGCGGTCTTATGCCGGCTGC |
| | | | TCTAGAACAACCCCTCCTCCTCGGTGTTTCGCCGAGG |
| | | | CCAGAATGGAACCTGTTCCTGGACTGGCCTGGCTGG |
| | | | CCTCCACAGTGAACCTGGAATTTCAGCACGCCTCTC |
| | | | CACAGCACGCCGGCCTGTATCTGTGTGTGGTGTACG |
| | | | TGGACGATCACATCCACGCCTGGGGCCACATGACCA |
| | | | TCTCTACAGCCGCTCAGTACCGGAACGCCGTGGTTG |
| | | | AACAGCATCTGCCCCAGAGACAGCCCGAGCCTGTGG |
| | | | AACCTACAAGACCTCATGTTCGGGCCTGATAA |
| 14 | HSV-2 gE | Version 2 | CGCACCTCTTGGAAACGCGTTACTTCCGGGGAGGAC |
| | [US8] | | GTTGTCCTCCTTCCAGCACCCGCAGGACCTGAGGAA |
| | | | AGGACTAGGGCCCACAAGCTGCTGTGGGCCGCTGAA |
| | | | CCTCTGGATGCCTGTGGTCCTCTGAGACCTAGCTGG |
| | | | GTCGCCCTTTGGCCACCTAGACGCGTTCTGGAGACG |
| | | | GTCGTGGATGCCGCGTGCATGCGTGCACCCGAACCT |
| | | | CTGGCCATCGCCTATAGTCCCCCTTTTCCCGCTGGCG |
| | | | ACGAGGGGCTTTACTCCGAACTGGCCTGGCGGGATA |
| | | | GGGTGGCGGTGGTGAACGAGAGCCTCGTCATCTACG |
| | | | GTGCTCTGGAAACCGACTCAGGACTGTATACGCTCA |
| | | | GCGTTGTTGGCCTCTCCGATGAGGCTCGACAGGTTG |
| | | | CCTCCGTAGTGCTGGTCGTAGAACCAGCCCCCGTAC |
| | | | CAACACCCACACCCGACGACTACGACGAGGAGGAC |
| | | | GACGCTGGAGTTAGCGAAAGAACACCGGTGAGTGT |
| | | | GCCACCTCCCACACCGCCAAGGAGACCCCCAGTAGC |
| | | | ACCTCCAACCCATCCGAGAGTGATTCCCGAGGTCAG |
| | | | CCATGTGCGCGGCGTAACTGTGCACATGGAGACGCC |
| | | | CGAAGCGATACTGTTTGCCCCTGGAGAGACATTCGG |
| | | | CACCAATGTGTCCATACACGCAATTGCGCACGATGA |
| | | | TGGCCCATACGCTATGGACGTCGTCTGGATGAGGTT |
| | | | CGATGTGCCTTCTTCTTGCGCCGAGATGAGGATCTAC |
| | | | GAGGCATGCCTGTATCACCCCCAATTGCCGGAGTGT |
| | | | CTGTCTCCCGCAGATGCACCGTGTGCAGTGAGTAGC |
| | | | TGGGCTTATCGGTTGGCTGTCCGGAGTTATGCTGGGT |
| | | | GTTCACGGACCACCCCACCTCCACGTTGCTTTGCTGA |
| | | | AGCCAGAATGGAACCCGTGCCTGGTCTGGCTTGGCT |
| | | | GGCATCAACTGTCAACCTGGAGTTCCAGCATGCCTC |
| | | | TCCACAGCACGCAGGCCTGTATCTCTGCGTGGTGTA |
| | | | CGTTGACGATCACATCCATGCGTGGGGGCATATGAC |
| | | | CATCAGCACAGCTGCCCAGTACCGCAATGCCGTCGT |
| | | | GGAGCAGCACCTCCCCCAACGGCAGCCAGAACCAGT |
| | | | GGAGCCCACTCGGCCTCATGTGCGAGCCTGA |
| 146 | HSV-2 gE | Version | CGCACCTCTTGGAAACGCGTTACTTCCGGGGAGGAC |
| | [US8] | 2.1 | GTTGTCCTCCTTCCAGCACCCGCAGGACCTGAAGAG |
| | | | AGGACTAGGGCCCACAAGCTGCTGTGGGCCGCTGAA |
| | | | CCTCTGGATGCCTGTGGTCCTCTGAGACCTAGCTGG |
| | | | GTCGCCCTTTGGCCACCTAGACGCGTTCTGGAGACG |
| | | | GTCGTGGATGCCGCGTGCATGCGTGCACCCGAACCT |
| | | | CTGGCCATCGCCTATAGTCCCCCTTTTCCCGCTGGCG |
| | | | ACGAGGGGCTTTACTCCGAACTGGCCTGGCGGGATA |
| | | | GGGTGGCGGTGGTGAACGAGAGCCTCGTCATCTACG |
| | | | GTGCTCTGGAAACCGACTCAGGACTGTATACGCTCA |
| | | | GCGTTGTTGGCCTCTCCGATGAGGCTCGACAGGTTG |
| | | | CCTCCGTAGTGCTGGTCGTAGAACCAGCCCCCGTAC |
| | | | CAACACCCACACCCGACGACTACGACGAAGAGGAC |
| | | | GACGCTGGAGTTAGCGAAAGAACACCGGTGAGTGT |
| | | | GCCACCTCCCACACCGCCAAGGAGACCCCCAGTAGC |
| | | | ACCTCCAACCCATCCGAGAGTGATTCCCGAGGTCAG |
| | | | CCATGTGCGCGGCGTAACTGTGCACATGGAGACGCC |
| | | | CGAAGCGATACTGTTTGCCCCTGGAGAGACATTCGG |
| | | | CACCAATGTGTCCATACACGCAATTGCGCACGATGA |
| | | | TGGCCCATACGCTATGGACGTCGTCTGGATGAGGTT |
| | | | CGATGTGCCTTCTTCTTGCGCCGAGATGAGGATCTAC |
| | | | GAGGCATGCCTGTATCACCCCCAATTGCCGGAGTGT |
| | | | CTGTCTCCCGCAGATGCACCGTGTGCAGTGAGTAGC |
| | | | TGGGCTTATCGGTTGGCTGTCCGGAGTTATGCTGGGT |
| | | | GTTCACGGACCACCCCACCTCCACGTTGCTTTGCTGA |
| | | | AGCCAGAATGGAACCCGTGCCTGGTCTGGCTTGGCT |
| | | | GGCATCAACTGTCAACCTGGAGTTCCAGCATGCCTC |
| | | | TCCACAGCACGCAGGCCTGTATCTCTGCGTGGTGTA |
| | | | CGTTGACGATCACATCCATGCGTGGGGGCATATGAC |
| | | | CATCAGCACAGCTGCCCAGTACCGCAATGCCGTCGT |
| | | | GGAGCAGCACCTCCCCCAACGGCAGCCAGAACCAGT |
| | | | GGAGCCCACTCGGCCTCATGTGCGAGCCTGA |
| 271 | HSV-2 gE | Version | CGCACCTCTTGGAAACGCGTTACTTCCGGGGAGGAC |
| | [US8] | 2.2 | GTTGTCCTCCTTCCAGCACCCGCAGGACCTGAGGAA |
| | | | AGGACTAGGGCCCACAAGCTGCTGTGGGCCGCTGAA |
| | | | CCTCTGGATGCCTGTGGTCCTCTGAGACCTAGCTGG |
| | | | GTCGCCCTTTGGCCACCTAGACGCGTTCTGGAGACG |
| | | | GTCGTGGATGCCGCGTGCATGCGTGCACCCGAACCT |
| | | | CTGGCCATCGCCTATAGTCCCCCTTTTCCCGCTGGCG |
| | | | ACGAGGGGCTTTACTCCGAACTGGCCTGGCGGGATA |
| | | | GGGTGGCGGTGGTGAACGAGAGCCTCGTCATCTACG |
| | | | GTGCTCTGGAAACCGACTCAGGACTGTATACGCTCA |
| | | | GCGTTGTTGGCCTCTCCGATGAGGCTCGACAGGTTG |
| | | | CCTCCGTAGTGCTGGTCGTAGAACCAGCCCCCGTAC |
| | | | CAACACCCACACCCGACGACTACGACGAGGAGGAC |
| | | | GACGCTGGAGTTAGCGAAAGAACACCGGTGAGTGT |
| | | | GCCACCTCCCACACCGCCAAGGAGACCCCCAGTAGC |
| | | | ACCTCCAACCCATCCGAGAGTGATTCCCGAGGTCAG |
| | | | CCATGTGCGCGGCGTAACTGTGCACATGGAGACGCC |
| | | | CGAAGCGATACTGTTTGCCCCTGGAGAGACATTCGG |
| | | | CACCAATGTGTCCATACACGCAATTGCGCACGATGA |
| | | | TGGCCCATACGCTATGGACGTCGTCTGGATGAGGTT |
| | | | CGATGTGCCTTCTTCTTGCGCCGAGATGAGGATCTAC |
| | | | GAGGCATGCCTGTATCACCCCCAATTGCCGGAGTGT |
| | | | CTGTCTCCCGCAGATGCACCGTGTGCAGTGAGTAGC |
| | | | TGGGCTTATCGGTTGGCTGTCCGGAGTTATGCTGGGT |
| | | | GTTCACGGACCACCCCACCTCCACGTTGCTTTGCTGA |
| | | | AGCCAGAATGGAACCCGTGCCTGGTCTGGCTTGGCT |
| | | | GGCATCAACTGTCAACCTGGAGTTCCAGCATGCCTC |
| | | | TCCACAGCACGCAGGCCTGTATCTCTGCGTGGTGTA |
| | | | CGTTGACGATCACATCCATGCGTGGGGGCATATGAC |
| | | | CATCAGCACAGCTGCCCAGTACCGCAATGCCGTCGT |
| | | | GGAGCAGCACCTCCCCCAACGGCAGCCAGAACCAGT |
| | | | GGAGCCCACTCGGCCTCATGTGCGAGCCTGATAA |
| 335 | HSV-2 gE | Version | CGCACCTCTTGGAAACGCGTTACTTCCGGGGAGGAC |
| | [US8] | 2.2 | GTTGTCCTCCTTCCAGCACCCGCAGGACCTGAGGAA |
| | | | AGGACTAGGGCCCACAAGCTGCTGTGGGCCGCTGAA |
| | | | CCTCTGGATGCCTGTGGTCCTCTGAGACCTAGCTGG |
| | | | GTCGCCCTTTGGCCACCTAGACGCGTTCTGGAGACG |
| | | | GTCGTGGATGCCGCGTGCATGCGTGCACCCGAACCT |
| | | | CTGGCCATCGCCTATAGTCCCCCTTTTCCCGCTGGCG |
| | | | ACGAGGGGCTTTACTCCGAACTGGCCTGGCGGGATA |
| | | | GGGTGGCGGTGGTGAACGAGAGCCTCGTCATCTACG |
| | | | GTGCTCTGGAAACCGACTCAGGACTGTATACGCTCA |
| | | | GCGTTGTTGGCCTCTCCGATGAGGCTCGACAGGTTG |
| | | | CCTCCGTAGTGCTGGTCGTAGAACCAGCCCCCGTAC |
| | | | CAACACCCACACCCGACGACTACGACGAGGAGGAC |
| | | | GACGCTGGAGTTAGCGAAAGAACACCGGTGAGTGT |
| | | | GCCACCTCCCACACCGCCAAGGAGACCCCCAGTAGC |
| | | | ACCTCCAACCCATCCGAGAGTGATTCCCGAGGTCAG |
| | | | CCATGTGCGCGGCGTAACTGTGCACATGGAGACGCC |
| | | | CGAAGCGATACTGTTTGCCCCTGGAGAGACATTCGG |
| | | | CACCAATGTGTCCATACACGCAATTGCGCACGATGA |
| | | | TGGCCCATACGCTATGGACGTCGTCTGGATGAGGTT |
| | | | CGATGTGCCTTCTTCTTGCGCCGAGATGAGGATCTAC |
| | | | GAGGCATGCCTGTATCACCCCCAATTGCCGGAGTGT |
| | | | CTGTCTCCCGCAGATGCACCGTGTGCAGTGAGTAGC |
| | | | TGGGCTTATCGGTTGGCTGTCCGGAGTTATGCTGGGT |
| | | | GTTCACGGACCACCCCACCTCCACGTTGCTTTGCTGA |
| | | | AGCCAGAATGGAACCCGTGCCTGGTCTGGCTTGGCT |
| | | | GGCATCAACTGTCAACCTGGAGTTCCAGCATGCCTC |
| | | | TCCACAGCACGCAGGCCTGTATCTCTGCGTGGTGTA |
| | | | CGTTGACGATCACATCCATGCGTGGGGGCATATGAC |
| | | | CATCAGCACAGCTGCCCAGTACCGCAATGCCGTCGT |
| | | | GGAGCAGCACCTCCCCCAACGGCAGCCAGAACCAGT |
| | | | GGAGCCCACTCGGCCTCATGTGCGAGCCTGATAA |
| 15 | HSV-2 gE | Version 3 | AGAACCTCCTGGAAAAGAGTGACCTCCGGCGAAGAT |
| | [US8] | | GTGGTGCTGCTGCCCGCCCCCGCCGGCCCCGAAGAA |
| | | | AGAACCAGAGCCCACAAACTGCTGTGGGCCGCCGA |
| | | | ACCCCTGGATGCCTGCGGCCCCCTCAGACCCTCCTG |
| | | | GGTGGCCCTGTGGCCCCCTAGAAGGGTGCTGGAAAC |
| | | | CGTGGTGGATGCCGCCTGCATGAGAGCCCCCGAACC |
| | | | CCTGGCCATCGCCTACTCCCCTCCCTTCCCCGCCGGC |
| | | | GATGAAGGCCTGTACTCCGAACTGGCCTGGAGAGAT |
| | | | AGAGTGGCCGTGGTGAACGAATCCCTGGTGATCTAC |
| | | | GGCGCCCTGGAAACCGATTCCGGCCTGTACACCCTG |
| | | | TCCGTGGTGGGCCTGTCCGATGAAGCCAGACAGGTG |
| | | | GCCTCCGTGGTGCTGGTGGTGGAACCCGCCCCCGTG |
| | | | CCCACCCCCACCCCCGATGATTACGATGAAGAAGAT |
| | | | GATGCCGGCGTGTCCGAAAGAACCCCCGTGTCCGTG |
| | | | CCCCCTCCCACCCCTCCCCGCAGACCCCCTGTGGCCC |
| | | | CTCCCACCCACCCCAGAGTGATCCCCGAAGTGTCCC |
| | | | ACGTGAGAGGCGTGACCGTGCACATGGAAACCCCCG |
| | | | AAGCCATCCTGTTCGCCCCCGGCGAAACCTTCGGCA |
| | | | CCAACGTGTCCATCCACGCCATCGCCCACGATGATG |
| | | | GCCCCTACGCCATGGATGTGGTGTGGATGAGATTCG |
| | | | ATGTGCCCTCCTCCTGCGCCGAAATGAGAATCTACG |
| | | | AAGCCTGCCTGTACCACCCCCAGCTGCCCGAATGCC |
| | | | TGTCCCCCGCCGATGCCCCCTGCGCCGTGTCCTCCTG |
| | | | GGCCTACAGACTGGCCGTGAGATCCTACGCCGGCTG |
| | | | CTCCAGAACCACCCCTCCCCCTAGATGCTTCGCCGA |
| | | | AGCCAGAATGGAACCCGTGCCCGGCCTGGCCTGGCT |
| | | | GGCCTCCACCGTGAACCTGGAATTCCAGCACGCCAG |
| | | | CCCCCAGCACGCCGGCCTGTACCTGTGCGTGGTGTA |
| | | | CGTGGATGATCACATCCACGCCTGGGGCCACATGAC |
| | | | CATCTCCACCGCCGCCCAGTACAGAAACGCCGTGGT |
| | | | GGAACAGCACCTGCCCCAGAGACAGCCCGAACCCGT |
| | | | GGAACCCACCAGACCCCACGTGAGAGCCTGA |
| 272 | HSV-2 gE | Version | AGAACCTCTTGGAAAAGAGTGACCTCTGGAGAAGAT |
| | [US8] | 3.1 | GTGGTGCTGCTGCCAGCTCCAGCTGGACCAGAAGAA |
| | | | AGAACCAGAGCACACAAACTGCTGTGGGCTGCTGAA |
| | | | CCTCTGGATGCTTGTGGACCTCTGAGACCTTCTTGGG |
| | | | TGGCTCTGTGGCCACCAAGAAGGGTGCTGGAAACAG |
| | | | TGGTGGATGCTGCTTGCATGAGAGCACCTGAACCTC |
| | | | TGGCAATTGCATACTCTCCTCCTTTTCCAGCTGGAGA |
| | | | TGAAGGACTGTATTCTGAACTGGCTTGGAGAGACAG |
| | | | AGTGGCTGTGGTGAATGAATCTCTGGTGATCTATGG |
| | | | AGCACTGGAAACAGATTCTGGACTGTACACCCTGTC |
| | | | TGTGGTGGGACTGTCTGATGAAGCAAGACAGGTGGC |
| | | | ATCTGTGGTGCTGGTGGTGGAACCAGCTCCAGTGCC |
| | | | AACCCCAACCCCAGATGATTATGATGAAGAAGATGA |
| | | | TGCTGGAGTGTCTGAAAGAACCCCAGTGTCTGTGCC |
| | | | ACCACCAACCCCACCAAGAAGACCACCAGTGGCTCC |
| | | | ACCAACCCACCCAAGAGTGATCCCAGAAGTGTCTCA |
| | | | TGTGAGAGGAGTGACAGTGCACATGGAAACCCCTGA |
| | | | AGCAATCCTGTTTGCACCTGGAGAAACCTTTGGAAC |
| | | | CAATGTGTCCATCCATGCAATTGCACATGATGATGG |
| | | | ACCTTATGCAATGGATGTGGTGTGGATGAGATTTGA |
| | | | TGTGCCTTCTTCTTGTGCTGAAATGAGAATCTATGAA |
| | | | GCTTGCCTGTACCACCCTCAGCTGCCTGAATGCCTGT |
| | | | CTCCTGCTGATGCTCCTTGTGCTGTGTCTTCTTGGGC |
| | | | TTACAGACTGGCTGTGAGATCTTATGCTGGATGCTCC |
| | | | AGAACCACCCCACCACCAAGATGCTTTGCTGAAGCA |
| | | | AGAATGGAACCTGTGCCTGGACTGGCATGGCTGGCA |
| | | | TCCACAGTGAATCTGGAATTTCAGCATGCTTCTCCTC |
| | | | AGCATGCTGGACTGTACCTGTGTGTGGTGTATGTGG |
| | | | ACGATCACATCCATGCTTGGGGACACATGACCATCA |
| | | | GCACAGCTGCTCAGTACAGAAATGCTGTGGTGGAAC |
| | | | AGCACCTGCCACAGAGACAGCCAGAACCAGTGGAA |
| | | | CCAACCAGACCACATGTGAGAGCTTGATAA |
| 273 | HSV-2 gE | Version 4 | CGGACCAGCTGGAAAAGGGTGACATCTGGGGAAGA |
| | [US8] | | TGTCGTGCTCCTTCCTGCGCCTGCTGGCCCAGAAGA |
| | | | ACGCACTAGGGCCCACAAGCTTCTGTGGGCCGCGGA |
| | | | GCCACTGGATGCGTGTGGTCCCCTGCGACCCTCTTG |
| | | | GGTGGCATTGTGGCCACCCCGACGCGTACTCGAAAC |
| | | | GGTCGTTGACGCCGCCTGTATGAGAGCGCCAGAGCC |
| | | | CCTCGCCATTGCCTACAGCCCGCCTTTTCCCGCCGGT |
| | | | GACGAAGGACTGTACAGTGAGCTGGCCTGGCGCGAT |
| | | | AGGGTCGCCGTAGTTAACGAGTCCCTGGTGATATAC |
| | | | GGCGCTCTGGAAACCGACAGTGGCTTGTACACCCTG |
| | | | TCCGTTGTAGGCCTGTCCGATGAAGCACGGCAAGTG |
| | | | GCTTCCGTGGTACTGGTCGTAGAGCCCGCACCAGTT |
| | | | CCCACACCCACCCCGGATGACTACGATGAGGAGGAC |
| | | | GATGCCGGTGTGAGTGAGCGTACACCTGTTAGCGTA |
| | | | CCTCCACCAACTCCTCCACGCCGCCCTCCTGTTGCAC |
| | | | CGCCTACACATCCCCGTGTGATTCCTGAAGTGTCAC |
| | | | ACGTTAGAGGGGTGACAGTCCACATGGAGACTCCCG |
| | | | AAGCCATCCTCTTTGCACCAGGCGAGACTTTTGGGA |
| | | | CCAATGTGAGCATTCACGCCATAGCTCACGATGACG |
| | | | GGCCCTATGCCATGGACGTGGTGTGGATGAGGTTCG |
| | | | ATGTGCCCTCATCATGCGCTGAGATGCGGATCTACG |
| | | | AAGCTTGCCTGTATCACCCACAGCTTCCCGAGTGCTT |
| | | | GTCTCCCGCTGACGCCCCTTGTGCTGTTAGCTCTTGG |
| | | | GCTTATCGGCTTGCCGTCAGGAGCTATGCTGGATGC |
| | | | TCCAGAACCACACCTCCACCGAGGTGTTTCGCCGAG |
| | | | GCCAGAATGGAGCCTGTGCCAGGACTGGCCTGGCTG |
| | | | GCAAGTACTGTGAACCTGGAATTCCAGCACGCATCA |
| | | | CCTCAGCATGCAGGCCTGTACCTCTGCGTTGTCTATG |
| | | | TCGACGACCATATCCACGCATGGGGCCATATGACCA |
| | | | TCAGCACGGCAGCACAGTATCGGAATGCTGTGGTCG |
| | | | AGCAGCACTTGCCCCAGCGACAACCCGAACCAGTGG |
| | | | AGCCAACCAGACCGCATGTGCGGGCCTGA |
| 142 | HSV-2 gD | Version 2 | GATCCGAGCCTCAAGATGGCAGATCCCAACCGATTT |
| | (US6) | EAM | CGGGGAAAGAATCTGCCAGTACTGGACCAGCTGACG |
| | | | GACCCACCTGGCGTCAAACGCGTCTACCACATACAG |
| | | | CCTAGTCTTGAGGACCCTTTTCAGCCACCGTCTATCC |
| | | | CCATTACCGTGTACTATGCCGTGCTGGAACGCGCGT |
| | | | GTAGGTCAGTTCTGCTGCATGCCCCATCCGAAGCCC |
| | | | CCCAGATCGTCAGAGGAGCTTCTGATGAAGCACGCA |
| | | | AACACACCTACAACCTCACAATAGCGTGGTATCGAA |
| | | | TGGGCGATAACTGCGCAATTCCCATCACAGTCATGG |
| | | | AGTACACGGAGTGCCCCTACAACAAGAGCCTCGGTG |
| | | | TTTGCCCTATCAGGACACAACCCAGGTGGAGCTATT |
| | | | ACGACAGTTTCAGCGCCGTGTCTGAGGACAATCTGG |
| | | | GGTTTCTGATGCACGCACCCGCCTTCGAGACTGCCG |
| | | | GCACCTACTTGCGGCTGGTGAAGATCAACGACTGGA |
| | | | CTGAGATCACCCAGTTCATCCTGGAACATAGGGCCA |
| | | | GAGCCAGCTGCAAGTATGCCCTTCCCCTGCGGATTC |
| | | | CGCCTGCAGCATGTCTGACCTCAAAAGCCTACCAGC |
| | | | AAGGGGTGACTGTGGACAGCATTGGCATGCTGCCTC |
| | | | GTTTCATTCCCGAGAATCAACGGACAGTGGCTCTGT |
| | | | ATTCCCTGAAGATCGCAGGATGGCATGGGCCCAAAC |
| | | | CACCTTATACCTCTACGTTGCTTCCACCAGAACTCAG |
| | | | TGACACCACTAATGCGACACAGCCAGAACTTGTGCC |
| | | | TGAGGATCCTGAAGATAGCGCTCTGTTGGAGGATCC |
| | | | AGCCGGTACTGTGTCCTCCCAGATACCACCCAATTG |
| | | | GCACATTCCTTCCATTCAGGACGTAGCTCCGCATCAC |
| | | | TGA |

TABLE 6
Example Ribonucleic Acid Sequences for HSV Glycoproteins

| SEQ ID | Antigen/ | | |
|---|---|---|---|
| NO | ORF | Versions | Polynucleotide Sequence |
| 16 | HSV-2 gC | | GCCUCCCCCGGCCGCACCAUCACCGUGGGCCCCCGC |
| | [UL44] | | GGCAACGCCUCCAACGCCGCCCCCUCCGCCUCCCCC |
| | | | CGCAACGCCUCCGCCCCCCGCACCACCCCCACCCCC |
| | | | CCCCAGCCCCGCAAGGCCACCAAGUCCAAGGCCUCC |
| | | | ACCGCCAAGCCCGCCCCCCCCCCCAAGACCGGCCCC |
| | | | CCCAAGACCUCCUCCGAGCCCGUGCGCUGCAACCG |
| | | | CCACGACCCCCUGGCCCGCUACGGCUCCCGCGUGCA |
| | | | GAUCCGCUGCCGCUUCCCCAACUCCACCCGCACCGA |
| | | | GUUCCGCCUGCAGAUCUGGCGCUACGCCACCGCCA |
| | | | CCGACGCCGAGAUCGGCACCGCCCCCUCCCUGGAG |
| | | | GAGGUGAUGGUGAACGUGUCCGCCCCCCCCGGCGG |
| | | | CCAGCUGGUGUACGACUCCGCCCCCAACCGCACCG |
| | | | ACCCCCACGUGAUCUGGGCCGAGGGCGCCGGCCCC |
| | | | GGCGCCUCCCCCCGCCUGUACUCCGUGGUGGGCCC |
| | | | CCUGGGCCGCCAGCGCCUGAUCAUCGAGGAGCUGA |
| | | | CCCUGGAGACCCAGGGCAUGUACUACUGGGUGUGG |
| | | | GGCCGCACCGACCGCCCCUCCGCCUACGGCACCUGG |
| | | | GUGCGCGUGCGCGUGUUCCGCCCCCCCUCCCUGAC |
| | | | CAUCCACCCCCACGCCGUGCUGGAGGGCCAGCCCU |
| | | | UCAAGGCCACCUGCACCGCCGCCACCUACUACCCCG |
| | | | GCAACCGCGCCGAGUUCGUGUGGUUCGAGGACGGC |
| | | | CGCCGCGUGUUCGACCCCGCCCAGAUCCACACCCA |
| | | | GACCCAGGAGAACCCCGACGGCUUCUCCACCGUGU |
| | | | CCACCGUGACCUCCGCCGCCGUGGGCGGCCAGGGC |
| | | | CCCCCCCGCACCUUCACCUGCCAGCUGACCUGGCAC |
| | | | CGCGACUCCGUGUCCUUCUCCCGCCGCAACGCCUCC |
| | | | GGCACCGCCUCCGUGCUGCCCCGCCCCACCAUCACC |
| | | | AUGGAGUUCACCGGCGACCACGCCGUGUGCACCGC |
| | | | CGGCUGCGUGCCCGAGGGCGUGACCUUCGCCUGGU |
| | | | UCCUGGGCGACGACUCCUCCCCCGCCGAGAAGGUG |
| | | | GCCGUGGCCUCCCAGACCUCCUGCGGCCGCCCCGGC |
| | | | ACCGCCACCAUCCGCUCCACCCUGCCCGUGUCCUAC |
| | | | GAGCAGACCGAGUACAUCUGCCGCCUGGCCGGCUA |
| | | | CCCCGACGGCAUCCCCGUGCUGGAGCACCACUAA |
| 17 | HSV-2 gC | Version 1 | AGCGCUUCUCCCGGCAGAACCAUCACAGUGGGCCC |
| | [UL44] | | UAGAGGCAACGCCUCUAAUGCCGCUCCUAGCGCCU |
| | | | CUCCUAGAAACGCCUCUGCUCCCAGAACCACACCU |
| | | | ACACCUCCACAGCCUAGAAAGGCCACCAAGAGCAA |
| | | | GGCCAGCACAGCCAAACCUGCUCCUCCACCUAAGA |
| | | | CAGGCCCUCCAAAGACAAGCUCUGAGCCCGUGCGG |
| | | | UGCAACAGACACGAUCCACUGGCCAGAUACGGCAG |
| | | | CCGGGUGCAGAUCAGAUGCAGAUUCCCCAACAGCA |
| | | | CCCGGACCGAGUUCCGGCUCCAGAUUUGGAGAUAC |
| | | | GCCACCGCCACAGAUGCCGAGAUUGGAACAGCCCC |
| | | | UAGCCUGGAAGAAGUGAUGGUCAACGUUUCAGCCC |
| | | | CUCCUGGCGGCCAGCUGGUGUAUGAUUCUGCCCCU |
| | | | AACCGGACCGAUCCUCACGUGAUAUGGGCUGAAGG |
| | | | UGCUGGCCCAGGCGCAAGCCCUAGACUGUAUUCUG |
| | | | UUGUGGGCCCUCUGGGCAGACAGCGGCUGAUCAUU |
| | | | GAGGAACUGACCCUGGAAACCCAGGGCAUGUACUA |
| | | | CUGGGUCUGGGGCAGAACCGAUAGACCAAGCGCCU |
| | | | AUGGCACCUGGGUUCGAGUGCGAGUGUUCAGACCU |
| | | | CCUAGCCUGACCAUCCAUCCUCACGCCGUUCUGGA |
| | | | AGGCCAGCCUUUCAAGGCCACAUGUACCGCCGCCA |
| | | | CCUACUAUCCCGGAAACAGAGCCGAGUUCGUUUGG |
| | | | UUCGAGGACGGCAGAAGGGUGUUCGACCCCGCUCA |
| | | | GAUCCACACACAGACCCAAGAGAACCCCGACGGCU |
| | | | UUAGCACCGUGUCCACAGUGACAUCUGCCGCCGUU |
| | | | GGAGGACAGGGCCCUCCUAGAACCUUUACCUGCCA |
| | | | GCUGACCUGGCACAGAGACAGCGUGUCCUUCAGCA |
| | | | GAAGAAACGCCAGCGGCACAGCCAGCGUUCUGCCU |
| | | | AGACCUACCAUCACCAUGGAAUUCACCGGCGACCA |
| | | | CGCCGUGUGUACAGCUGGAUGUGUUCCUGAGGGCG |
| | | | UGACCUUCGCUUGGUUUCUGGGCGACGAUAGCAGC |
| | | | CCUGCCGAAAAAGUGGCUGUGGCCAGCCAGACAAG |
| | | | CUGUGGCAGACCUGGAACCGCCACCAUCAGAAGCA |
| | | | CACUGCCUGUCAGCUACGAGCAGACCGAGUACAUC |
| | | | UGUCGGCUGGCCGGCUAUCCUGAUGGCAUCCCUGU |
| | | | GCUGGAACACCACUGA |
| 274 | HSV-2 gC | Version | AGCCCUGGCAGAACCAUCACAGUGGGCCCUAGAGG |
| | [UL44] | 1.1 | CAACGCCUCUAAUGCCGCUCCUAGCGCCUCUCCUA |
| | | | GAAACGCCUCUGCUCCCAGAACCACACCUACACCU |
| | | | CCACAGCCUAGAAAGGCCACCAAGAGCAAGGCCAG |
| | | | CACAGCCAAACCUGCUCCUCCACCUAAGACAGGCC |
| | | | CUCCAAAGACAAGCUCUGAGCCCGUGCGGUGCAAC |
| | | | AGACACGAUCCACUGGCCAGAUACGGCAGCCGGGU |
| | | | GCAGAUCAGAUGCAGAUUCCCCAACAGCACCCGGA |
| | | | CCGAGUUCCGGCUCCAGAUUUGGAGAUACGCCACC |
| | | | GCCACAGAUGCCGAGAUUGGAACAGCCCCUAGCCU |
| | | | GGAAGAAGUGAUGGUCAACGUUUCAGCCCCUCCUG |
| | | | GCGGCCAGCUGGUGUAUGAUUCUGCCCCUAACCGG |
| | | | ACCGAUCCUCACGUGAUAUGGGCUGAAGGUGCUGG |
| | | | CCCUGGCGCUUCCCCUAGACUGUAUUCUGUUGUGG |
| | | | GCCCUCUGGGCAGACAGCGGCUGAUCAUUGAGGAA |
| | | | CUGACCCUGGAAACCCAGGGCAUGUACUACUGGGU |
| | | | CUGGGGCAGAACCGAUAGACCAAGCGCCUAUGGCA |
| | | | CCUGGGUUCGAGUGCGAGUGUUCAGACCUCCUAGC |
| | | | CUGACCAUCCAUCCUCACGCCGUUCUGGAAGGCCA |
| | | | GCCUUUCAAGGCCACAUGUACCGCCGCCACCUACU |
| | | | AUCCCGGAAACAGAGCCGAGUUCGUUUGGUUCGAG |
| | | | GACGGCAGAAGGGUGUUCGACCCCGCUCAGAUCCA |
| | | | CACACAGACCCAAGAGAACCCCGACGGCUUUAGCA |
| | | | CCGUGUCCACAGUGACAUCUGCCGCCGUUGGAGGA |
| | | | CAGGGCCCUCCUAGAACCUUUACCUGCCAGCUGAC |
| | | | CUGGCACAGAGACAGCGUGUCCUUCAGCAGAAGAA |
| | | | ACGCCAGCGGCACAGCCAGCGUUCUGCCUAGACCU |
| | | | ACCAUCACCAUGGAAUUCACCGGCGACCACGCCGU |
| | | | GUGUACAGCUGGAUGUGUUCCUGAGGGCGUGACCU |
| | | | UCGCUUGGUUUCUGGGCGACGAUAGCAGCCCUGCC |
| | | | GAAAAAGUGGCUGUGGCCAGCCAGACAAGCUGUGG |
| | | | CAGACCUGGAACCGCCACCAUCAGAAGCACACUGC |
| | | | CUGUCAGCUACGAGCAGACCGAGUACAUCUGUCGG |
| | | | CUGGCCGGCUAUCCUGAUGGCAUCCCUGUGCUGGA |
| | | | ACACCACUGAUAA |
| 18 | HSV-2 gC | Version 2 | GCAAGCCCCGGCAGAACCAUAACAGUAGGGCCACG |
| | [UL44] | | GGGGAAUGCUUCCAAUGCUGCACCUUCCGCUUCAC |
| | | | CGAGGAAUGCUUCUGCCCCAAGAACUACCCCCACU |
| | | | CCUCCUCAACCCAGGAAAGCGACAAAGUCCAAGGC |
| | | | CAGCACCGCAAAACCCGCUCCUCCUCCAAAGACUG |
| | | | GGCCCCCAAAGACAAGUAGCGAACCAGUUCGGUGC |
| | | | AACAGGCAUGACCCACUUGCACGCUAUGGGUCAAG |
| | | | AGUCCAGAUACGGUGUCGCUUCCCUAACAGUACAA |
| | | | GGACUGAGUUUCGGCUGCAGAUCUGGCGUUAUGCC |
| | | | ACAGCUACUGACGCAGAGAUUGGUACCGCCCCCAG |
| | | | UUUGGAGGAAGUGAUGGUCAACGUGUCCGCACCCC |
| | | | CAGGAGGACAGCUGGUCUAUGACUCAGCGCCCAAU |
| | | | AGGACCGAUCCCCACGUGAUCUGGGCAGAAGGAGC |
| | | | CGGUCCUGGGGCCUCUCCACGGCUGUACUCAGUUG |
| | | | UUGGCCCGCUUGGACGACAGAGACUCAUCAUCGAG |
| | | | GAACUGACACUGGAGACACAGGGGAUGUACUACUG |
| | | | GGUGUGGGGCCGUACUGACCGCCCUUCCGCAUAUG |
| | | | GCACUUGGGUGAGAGUUCGCGUCUUUCGGCCCCCU |
| | | | UCUCUCACCAUCCAUCCUCAUGCCGUGCUCGAAGG |
| | | | CCAGCCCUUUAAGGCCACAUGCACUGCUGCGACCU |
| | | | ACUACCCUGGCAACAGAGCCGAGUUUGUCUGGUUU |
| | | | GAGGAUGGUCGGCGAGUAUUCGAUCCAGCCCAGAU |
| | | | UCACACACAAACGCAGGAAAAUCCGGACGGCUUCA |
| | | | GCACAGUGUCCACGGUGACCUCUGCUGCAGUUGGU |
| | | | GGACAAGGACCCCCUCGAACCUUCACCUGUCAGCU |
| | | | GACCUGGCACAGAGACUCCGUAAGCUUCAGCCGUA |
| | | | GAAACGCCUCUGGAACCGCCAGUGUGUUGCCGAGG |
| | | | CCGACUAUCACGAUGGAAUUCACAGGCGAUCAUGC |
| | | | CGUCUGUACUGCCGGCUGUGUGCCAGAAGGCGUAA |
| | | | CCUUCGCUUGGUUUCUCGGGGAUGACUCAAGUCCU |
| | | | GCAGAGAAAGUGGCUGUGGCCUCUCAGACGAGCUG |
| | | | CGGUCGACCAGGAACAGCUACCAUUCGCAGCACUC |
| | | | UGCCCGUGUCCUACGAGCAGACGGAGUACAUCUGC |
| | | | AGGCUGGCCGGCUAUCCCGAUGGGAUUCCAGUCCU |
| | | | GGAGCACCACUGA |
| 147 | HSV-2 gC | Version | GCAAGCCCCGGCAGAACCAUAACAGUAGGGCCACG |
| | [UL44] | 2.1 | GGGGAAUGCUUCCAAUGCUGCACCUUCCGCUUCAC |
| | | | CGAGGAAUGCUUCUGCCCCAAGAACUACCCCCACU |
| | | | CCUCCUCAACCCAGGAAAGCGACAAAGUCCAAGGC |
| | | | CAGCACCGCAAAACCCGCUCCUCCUCCAAAGACUG |
| | | | GGCCCCCAAAGACAAGUAGCGAACCAGUUCGGUGC |
| | | | AACAGGCAUGACCCACUUGCACGCUAUGGGUCAAG |
| | | | AGUCCAGAUACGGUGUCGCUUCCCUAACAGUACAA |
| | | | GGACUGAGUUUCGGCUGCAGAUCUGGCGUUAUGCC |
| | | | ACAGCUACUGACGCAGAGAUUGGUACCGCCCCCAG |
| | | | UUUGGAAGAGGUGAUGGUCAACGUGUCCGCACCCC |
| | | | CAGGAGGACAGCUGGUCUAUGACUCAGCGCCCAAU |
| | | | AGGACCGAUCCCCACGUGAUCUGGGCAGAAGGAGC |
| | | | CGGUCCUGGGGCCUCUCCACGGCUGUACUCAGUUG |
| | | | UUGGCCCGCUUGGACGACAGAGACUCAUCAUCGAA |
| | | | GAGCUGACACUGGAGACACAGGGGAUGUACUACUG |
| | | | GGUGUGGGGCCGUACUGACCGCCCUUCCGCAUAUG |
| | | | GCACUUGGGUGAGAGUUCGCGUCUUUCGGCCCCCU |
| | | | UCUCUCACCAUCCAUCCUCAUGCCGUGCUCGAAGG |
| | | | CCAGCCCUUUAAGGCCACAUGCACUGCUGCGACCU |
| | | | ACUACCCUGGCAACAGAGCCGAGUUUGUCUGGUUU |
| | | | GAGGAUGGUCGGCGAGUAUUCGAUCCAGCCCAGAU |
| | | | UCACACACAAACGCAGGAAAAUCCGGACGGCUUCA |
| | | | GCACAGUGUCCACGGUGACCUCUGCUGCAGUUGGU |
| | | | GGACAAGGACCCCCUCGAACCUUCACCUGUCAGCU |
| | | | GACCUGGCACAGAGACUCCGUAAGCUUCAGCCGUA |
| | | | GAAACGCCUCUGGAACCGCCAGUGUGUUGCCGAGG |
| | | | CCGACUAUCACGAUGGAAUUCACAGGCGAUCAUGC |
| | | | CGUCUGUACUGCCGGCUGUGUGCCAGAAGGCGUAA |
| | | | CCUUCGCUUGGUUUCUCGGGGAUGACUCAAGUCCU |
| | | | GCAGAGAAAGUGGCUGUGGCCUCUCAGACGAGCUG |
| | | | CGGUCGACCAGGAACAGCUACCAUUCGCAGCACUC |
| | | | UGCCCGUGUCCUACGAGCAGACGGAGUACAUCUGC |
| | | | AGGCUGGCCGGCUAUCCCGAUGGGAUUCCAGUCCU |
| | | | GGAGCACCACUGA |
| 275 | HSV-2 gC | Version | AGCCCCGGCAGAACCAUAACAGUAGGGCCACGGGG |
| | [UL44] | 2.2 | GAAUGCUUCCAAUGCUGCACCUUCCGCUUCACCGA |
| | | | GGAAUGCUUCUGCCCCAAGAACUACCCCCACUCCU |
| | | | CCUCAACCCAGGAAAGCGACAAAGUCCAAGGCCAG |
| | | | CACCGCAAAACCCGCUCCUCCUCCAAAGACUGGGC |
| | | | CCCCAAAGACAAGUAGCGAACCAGUUCGGUGCAAC |
| | | | AGGCAUGACCCACUUGCACGCUAUGGGUCAAGAGU |
| | | | CCAGAUACGGUGUCGCUUCCCUAACAGUACAAGGA |
| | | | CUGAGUUUCGGCUGCAGAUCUGGCGUUAUGCCACA |
| | | | GCUACUGACGCAGAGAUUGGUACCGCCCCCAGUUU |
| | | | GGAAGAGGUGAUGGUCAACGUGUCCGCACCCCCAG |
| | | | GAGGACAGCUGGUCUAUGACUCAGCGCCCAAUAGG |
| | | | ACCGAUCCCCACGUGAUCUGGGCAGAAGGAGCCGG |
| | | | UCCUGGGGCCUCUCCACGGCUGUACUCAGUUGUUG |
| | | | GCCCGCUUGGACGACAGAGACUCAUCAUCGAAGAG |
| | | | CUGACACUGGAGACACAGGGGAUGUACUACUGGGU |
| | | | GUGGGGCCGUACUGACCGCCCUUCCGCAUAUGGCA |
| | | | CUUGGGUGAGAGUUCGCGUCUUUCGGCCCCCUUCU |
| | | | CUCACCAUCCAUCCUCAUGCCGUGCUCGAAGGCCA |
| | | | GCCCUUUAAGGCCACAUGCACUGCUGCGACCUACU |
| | | | ACCCUGGCAACAGAGCCGAGUUUGUCUGGUUUGAG |
| | | | GAUGGUCGGCGAGUAUUCGAUCCAGCCCAGAUUCA |
| | | | CACACAAACGCAGGAAAAUCCGGACGGCUUCAGCA |
| | | | CAGUGUCCACGGUGACCUCUGCUGCAGUUGGUGGA |
| | | | CAAGGACCCCCUCGAACCUUCACCUGUCAGCUGAC |
| | | | CUGGCACAGAGACUCCGUAAGCUUCAGCCGUAGAA |
| | | | ACGCCUCUGGAACCGCCAGUGUGUUGCCGAGGCCG |
| | | | ACUAUCACGAUGGAAUUCACAGGCGAUCAUGCCGU |
| | | | CUGUACUGCCGGCUGUGUGCCAGAAGGCGUAACCU |
| | | | UCGCUUGGUUUCUCGGGGAUGACUCAAGUCCUGCA |
| | | | GAGAAAGUGGCUGUGGCCUCUCAGACGAGCUGCGG |
| | | | UCGACCAGGAACAGCUACCAUUCGCAGCACUCUGC |
| | | | CCGUGUCCUACGAGCAGACGGAGUACAUCUGCAGG |
| | | | CUGGCCGGCUAUCCCGAUGGGAUUCCAGUCCUGGA |
| | | | GCACCACUGAUAA |
| 290 | HSV-2 gC | Version | AGCCCCGGCAGAACCAUAACAGUAGGGCCACGGGG |
| | [UL44] | 2.3 | GAAUGCUUCCAAUGCUGCACCUUCCGCUUCACCGA |
| | | | GGAAUGCUUCUGCCCCAAGAACUACCCCCACUCCU |
| | | | CCUCAACCCAGGAAAGCGACAAAGUCCAAGGCCAG |
| | | | CACCGCAAAACCCGCUCCUCCUCCAAAGACUGGGC |
| | | | CCCCAAAGACAAGUAGCGAACCAGUUCGGUGCAAC |
| | | | AGGCAUGACCCACUUGCACGCUAUGGGUCAAGAGU |
| | | | CCAGAUACGGUGUCGCUUCCCUAACAGUACAAGGA |
| | | | CUGAGUUUCGGCUGCAGAUCUGGCGUUAUGCCACA |
| | | | GCUACUGACGCAGAGAUUGGUACCGCCCCCAGUUU |
| | | | GGAGGAAGUGAUGGUCAACGUGUCCGCACCCCCAG |
| | | | GAGGACAGCUGGUCUAUGACUCAGCGCCCAAUAGG |
| | | | ACAGACCCACAUGUUAUCUGGGCAGAAGGAGCCGG |
| | | | UCCUGGGGCCUCUCCACGGCUGUACUCAGUUGUUG |
| | | | GCCCGCUUGGACGACAGAGACUCAUCAUCGAGGAA |
| | | | CUGACACUGGAGACACAGGGGAUGUACUACUGGGU |
| | | | GUGGGGCCGUACUGACCGCCCUUCCGCAUAUGGCA |
| | | | CUUGGGUGAGAGUUCGCGUCUUUCGGCCCCCUUCU |
| | | | CUCACCAUCCAUCCUCAUGCCGUGCUCGAAGGCCA |
| | | | GCCCUUUAAGGCCACAUGCACUGCUGCGACCUACU |
| | | | ACCCUGGCAACAGAGCCGAGUUUGUCUGGUUUGAG |
| | | | GAUGGUCGGCGAGUAUUCGAUCCAGCCCAGAUUCA |
| | | | CACACAAACGCAGGAAAAUCCGGACGGCUUCAGCA |
| | | | CAGUGUCCACGGUGACCUCUGCUGCAGUUGGUGGA |
| | | | CAAGGACCCCCUCGAACCUUCACCUGUCAGCUGAC |
| | | | CUGGCACAGAGACUCCGUAAGCUUCAGCCGUAGAA |
| | | | ACGCCUCUGGAACCGCCAGUGUGUUGCCGAGGCCG |
| | | | ACUAUCACGAUGGAAUUCACAGGCGAUCAUGCCGU |
| | | | CUGUACUGCCGGCUGUGUGCCAGAAGGCGUAACCU |
| | | | UCGCUUGGUUUCUCGGGGAUGACUCAAGUCCUGCA |
| | | | GAGAAAGUGGCUGUGGCCUCUCAGACGAGCUGCGG |
| | | | UCGACCAGGAACAGCUACCAUUCGCAGCACUCUGC |
| | | | CCGUGUCCUACGAGCAGACGGAGUACAUCUGCAGG |
| | | | CUGGCCGGCUAUCCCGAUGGGAUUCCAGUCCUGGA |
| | | | GCACCACUGA |
| 291 | HSV-2 gC | Version | AGCCCCGGCAGAACCAUAACAGUAGGGCCACGGGG |
| | [UL44] | 2.4 | GAAUGCUUCCAAUGCUGCACCUUCCGCUUCACCGA |
| | | | GGAAUGCUUCUGCCCCAAGAACUACCCCCACUCCU |
| | | | CCUCAACCCAGGAAAGCGACAAAGUCCAAGGCCAG |
| | | | CACCGCAAAACCCGCUCCUCCUCCAAAGACUGGGC |
| | | | CCCCAAAGACAAGUAGCGAACCAGUUCGGUGCAAC |
| | | | AGGCAUGACCCACUUGCACGCUAUGGGUCAAGAGU |
| | | | CCAGAUACGGUGUCGCUUCCCUAACAGUACAAGGA |
| | | | CUGAGUUUCGGCUGCAGAUCUGGCGUUAUGCCACA |
| | | | GCUACUGACGCAGAGAUUGGUACCGCCCCCAGUUU |
| | | | GGAGGAAGUGAUGGUCAACGUGUCCGCACCCCCAG |
| | | | GAGGACAGCUGGUCUAUGACUCAGCGCCCAAUAGG |
| | | | ACCGAUCCCCACGUGAUCUGGGCAGAAGGAGCCGG |
| | | | UCCUGGGGCCUCUCCACGGCUGUACUCAGUUGUUG |
| | | | GCCCGCUUGGACGACAGAGACUCAUCAUCGAGGAA |
| | | | CUGACACUGGAGACACAGGGGAUGUACUACUGGGU |
| | | | GUGGGGCCGUACUGACCGCCCUUCCGCAUAUGGCA |
| | | | CUUGGGUGAGAGUUCGCGUCUUUCGGCCCCCUUCU |
| | | | CUCACCAUCCAUCCUCAUGCCGUGCUCGAAGGCCA |
| | | | GCCCUUUAAGGCCACAUGCACUGCUGCGACCUACU |
| | | | ACCCUGGCAACAGAGCCGAGUUUGUCUGGUUUGAG |
| | | | GAUGGUCGGCGAGUAUUCGAUCCAGCCCAGAUUCA |
| | | | CACACAAACGCAGGAAAAUCCGGACGGCUUCAGCA |
| | | | CAGUGUCCACGGUGACCUCUGCUGCAGUUGGUGGA |
| | | | CAAGGACCCCCUCGAACCUUCACCUGUCAGCUGAC |
| | | | CUGGCACAGAGACUCCGUAAGCUUCAGCCGUAGAA |
| | | | ACGCCUCUGGAACCGCCAGUGUGUUGCCGAGGCCG |
| | | | ACUAUCACGAUGGAAUUCACAGGCGAUCAUGCCGU |
| | | | CUGUACUGCCGGCUGUGUGCCAGAAGGCGUAACCU |
| | | | UCGCUUGGUUUCUCGGGGAUGACUCAAGUCCUGCA |
| | | | GAGAAAGUGGCUGUGGCCUCUCAGACGAGCUGCGG |
| | | | UCGACCAGGAACAGCUACCAUUCGCAGCACUCUGC |
| | | | CCGUGUCCUACGAGCAGACGGAGUACAUCUGCAGG |
| | | | CUGGCCGGCUAUCCCGAUGGGAUUCCAGUCCUGGA |
| | | | GCACCACUGA |
| 336 | HSV-2 gC | Version | AGCCCCGGCAGAACCAUAACAGUAGGGCCACGGGG |
| | [UL44] | 2.5 | GAAUGCUUCCAAUGCUGCACCUUCCGCUUCACCGA |
| | | | GGAAUGCUUCUGCCCCAAGAACUACCCCCACUCCU |
| | | | CCUCAACCCAGGAAAGCGACAAAGUCCAAGGCCAG |
| | | | CACCGCAAAACCCGCUCCUCCUCCAAAGACUGGGC |
| | | | CCCCAAAGACAAGUAGCGAACCAGUUCGGUGCAAC |
| | | | AGGCAUGACCCACUUGCACGCUAUGGGUCAAGAGU |
| | | | CCAGAUACGGUGUCGCUUCCCUAACAGUACAAGGA |
| | | | CUGAGUUUCGGCUGCAGAUCUGGCGUUAUGCCACA |
| | | | GCUACUGACGCAGAGAUUGGUACCGCCCCCAGUUU |
| | | | GGAGGAAGUGAUGGUCAACGUGUCCGCACCCCCAG |
| | | | GAGGACAGCUGGUCUAUGACUCAGCGCCCAAUAGG |
| | | | ACCGAUCCCCACGUGAUCUGGGCAGAAGGAGCCGG |
| | | | UCCUGGGGCCUCUCCACGGCUGUACUCAGUUGUUG |
| | | | GCCCGCUUGGACGACAGAGACUCAUCAUCGAGGAA |
| | | | CUGACACUGGAGACACAGGGGAUGUACUACUGGGU |
| | | | GUGGGGCCGUACUGACCGCCCUUCCGCAUAUGGCA |
| | | | CUUGGGUGAGAGUUCGCGUCUUUCGGCCCCCUUCU |
| | | | CUCACCAUCCAUCCUCAUGCCGUGCUCGAAGGCCA |
| | | | GCCCUUUAAGGCCACAUGCACUGCUGCGACCUACU |
| | | | ACCCUGGCAACAGAGCCGAGUUUGUCUGGUUUGAG |
| | | | GAUGGUCGGCGAGUAUUCGAUCCAGCCCAGAUUCA |
| | | | CACACAAACGCAGGAAAAUCCGGACGGCUUCAGCA |
| | | | CAGUGUCCACGGUGACCUCUGCUGCAGUUGGUGGA |
| | | | CAAGGACCCCCUCGAACCUUCACCUGUCAGCUGAC |
| | | | CUGGCACAGAGACUCCGUAAGCUUCAGCCGUAGAA |
| | | | ACGCCUCUGGAACCGCCAGUGUGUUGCCGAGGCCG |
| | | | ACUAUCACGAUGGAAUUCACAGGCGAUCAUGCCGU |
| | | | CUGUACUGCCGGCUGUGUGCCAGAAGGCGUAACCU |
| | | | UCGCUUGGUUUCUCGGGGAUGACUCAAGUCCUGCA |
| | | | GAGAAAGUGGCUGUGGCCUCUCAGACGAGCUGCGG |
| | | | UCGACCAGGAACAGCUACCAUUCGCAGCACUCUGC |
| | | | CCGUGUCCUACGAGCAGACGGAGUACAUCUGCAGG |
| | | | CUGGCCGGCUAUCCCGAUGGGAUUCCAGUCCUGGA |
| | | | GCACCACUGAUAA |
| 19 | HSV-2 gC | Version 3 | GCCUCCCCCGGCAGAACCAUCACCGUGGGCCCCAG |
| | [UL44] | | AGGCAACGCCUCCAACGCCGCCCCCUCCGCCUCCCC |
| | | | CAGAAACGCCUCCGCCCCCAGAACCACCCCCACCCC |
| | | | UCCCCAGCCCAGAAAAGCCACCAAAUCCAAAGCCU |
| | | | CCACCGCCAAACCCGCCCCUCCUCCCAAAACCGGCC |
| | | | CUCCCAAAACCUCCUCCGAACCCGUGAGAUGCAAC |
| | | | AGACACGAUCCCCUGGCCAGAUACGGCUCCAGAGU |
| | | | GCAGAUCAGAUGCAGAUUCCCCAACUCCACCAGAA |
| | | | CCGAAUUCAGACUGCAGAUCUGGAGAUACGCCACC |
| | | | GCCACCGAUGCCGAAAUCGGCACCGCCCCCUCCCU |
| | | | GGAAGAAGUGAUGGUGAACGUGUCCGCCCCUCCUG |
| | | | GCGGCCAGCUGGUGUACGAUUCCGCCCCCAACAGA |
| | | | ACCGAUCCCCACGUGAUCUGGGCCGAAGGCGCCGG |
| | | | CCCCGGCGCCUCCCCCAGACUGUACUCCGUGGUGG |
| | | | GCCCCCUGGGCAGACAGAGACUGAUCAUCGAAGAA |
| | | | CUGACCCUGGAAACCCAGGGCAUGUACUACUGGGU |
| | | | GUGGGGCAGAACCGAUAGACCCUCCGCCUACGGCA |
| | | | CCUGGGUGAGAGUGAGAGUGUUCAGACCCCCUUCC |
| | | | CUGACCAUCCACCCCCACGCCGUGCUGGAAGGCCA |
| | | | GCCCUUCAAAGCCACCUGCACCGCCGCCACCUACU |
| | | | ACCCCGGCAACAGAGCCGAAUUCGUGUGGUUCGAA |
| | | | GAUGGCAGAAGGGUGUUCGAUCCCGCCCAGAUCCA |
| | | | CACCCAGACCCAGGAAAACCCCGACGGCUUCUCCA |
| | | | CCGUGUCCACCGUGACCUCCGCCGCCGUGGGCGGC |
| | | | CAGGGCCCUCCCAGAACCUUCACCUGCCAGCUGAC |
| | | | CUGGCACAGAGACUCCGUGUCCUUCUCCAGAAGAA |
| | | | ACGCCUCCGGCACCGCCUCCGUGCUGCCCAGACCCA |
| | | | CCAUCACCAUGGAAUUCACCGGCGAUCACGCCGUG |
| | | | UGCACCGCCGGCUGCGUGCCCGAAGGCGUGACCUU |
| | | | CGCCUGGUUCCUGGGCGAUGAUUCCUCCCCCGCCG |
| | | | AAAAAGUGGCCGUGGCCUCCCAGACCUCCUGCGGC |
| | | | AGACCCGGCACCGCCACCAUCAGAUCCACCCUGCCC |
| | | | GUGUCCUACGAACAGACCGAAUACAUCUGCAGACU |
| | | | GGCCGGCUACCCCGAUGGCAUCCCCGUGCUGGAAC |
| | | | ACCACUGA |
| 276 | HSV-2 gC | Version | UCUCCUGGAAGAACCAUCACAGUGGGACCAAGAGG |
| | [UL44] | 3.1 | AAAUGCAAGCAAUGCAGCACCUUCUGCUUCUCCAA |
| | | | GAAAUGCUUCUGCUCCAAGAACCACCCCAACCCCU |
| | | | CCUCAGCCAAGAAAAGCAACCAAAUCCAAAGCAUC |
| | | | CACAGCAAAACCUGCACCUCCUCCAAAAACAGGAC |
| | | | CUCCAAAAACCUCCUCUGAACCUGUGAGAUGCAAC |
| | | | AGACAUGAUCCUCUGGCAAGAUAUGGAUCAAGAGU |
| | | | GCAGAUCAGAUGCAGAUUUCCAAAUUCCACCAGAA |
| | | | CAGAAUUCAGACUCCAGAUCUGGAGAUAUGCAACA |
| | | | GCAACAGAUGCAGAAAUUGGAACAGCACCAUCUCU |
| | | | GGAAGAAGUGAUGGUGAAUGUGUCUGCUCCUCCUG |
| | | | GAGGACAGCUGGUGUAUGAUUCUGCUCCAAACAGA |
| | | | ACAGAUCCUCAUGUGAUCUGGGCUGAAGGAGCUGG |
| | | | ACCUGGAGCUUCUCCAAGACUGUACUCUGUGGUGG |
| | | | GACCUCUGGGAAGACAGAGACUGAUCAUUGAAGAA |
| | | | CUGACCCUGGAAACCCAGGGAAUGUACUACUGGGU |
| | | | GUGGGGAAGAACAGACAGACCUUCUGCUUAUGGAA |
| | | | CCUGGGUGAGAGUGAGAGUGUUCAGACCUCCUUCU |
| | | | CUGACCAUCCACCCUCAUGCUGUGCUGGAAGGACA |
| | | | GCCUUUCAAAGCAACCUGCACAGCAGCAACCUACU |
| | | | ACCCUGGAAACAGAGCUGAAUUUGUGUGGUUUGA |
| | | | AGAUGGAAGAAGGGUGUUUGAUCCUGCUCAGAUCC |
| | | | ACACCCAGACCCAGGAAAAUCCUGAUGGAUUUUCC |
| | | | ACAGUGUCCACAGUGACAUCUGCUGCUGUGGGAGG |
| | | | ACAGGGACCUCCAAGAACCUUCACCUGCCAGCUGA |
| | | | CCUGGCACAGAGAUUCUGUGUCUUUUUCAAGAAGA |
| | | | AAUGCUUCUGGAACAGCUUCUGUGCUGCCAAGACC |
| | | | AACCAUCACCAUGGAAUUCACAGGAGAUCAUGCUG |
| | | | UGUGCACAGCUGGAUGUGUGCCUGAAGGAGUGACC |
| | | | UUUGCUUGGUUUCUGGGAGAUGAUUCUUCUCCAGC |
| | | | UGAAAAAGUGGCUGUGGCUUCCCAGACCUCUUGUG |
| | | | GAAGACCUGGAACAGCAACCAUCAGAUCCACCCUG |
| | | | CCUGUGUCUUAUGAACAGACAGAAUACAUUUGCAG |
| | | | ACUGGCUGGAUACCCUGAUGGAAUCCCUGUGCUGG |
| | | | AACACCACUGAUAA |
| 277 | HSV-2 gC | Version 4 | UCUCCGGGACGGACUAUAACCGUAGGUCCAAGAGG |
| | [UL44] | | AAACGCCUCUAACGCAGCCCCGUCUGCCUCACCAC |
| | | | GAAACGCCUCAGCUCCCAGAACCACUCCUACUCCA |
| | | | CCCCAGCCUAGGAAGGCGACGAAAUCCAAGGCUUC |
| | | | CACGGCCAAACCCGCCCCUCCACCCAAAACCGGACC |
| | | | UCCUAAGACCAGCUCUGAACCGGUGCGGUGUAAUA |
| | | | GGCACGACCCAUUGGCGCGAUAUGGCAGUAGGGUC |
| | | | CAGAUACGGUGCAGAUUCCCAAACAGCACAAGAAC |
| | | | AGAAUUCCGGCUGCAAAUCUGGCGAUAUGCAACGG |
| | | | CCACCGAUGCCGAAAUCGGAACAGCACCCAGUCUG |
| | | | GAAGAAGUGAUGGUGAACGUCAGUGCUCCACCUGG |
| | | | CGGACAACUGGUGUACGACUCUGCACCCAAUCGCA |
| | | | CAGAUCCCCACGUGAUUUGGGCCGAGGGUGCUGGA |
| | | | CCUGGGGCUUCACCCAGGCUGUAUAGCGUUGUAGG |
| | | | GCCACUUGGGAGGCAGAGACUCAUCAUUGAGGAAC |
| | | | UGACCCUGGAAACUCAGGGCAUGUACUACUGGGUA |
| | | | UGGGGCCGCACAGAUCGCCCCAGCGCUUAUGGCAC |
| | | | CUGGGUGCGGGUGCGGGUGUUUCGCCCACCCUCCC |
| | | | UCACCAUUCACCCUCAUGCGGUUCUGGAGGGACAG |
| | | | CCUUUCAAGGCAACUUGUACCGCAGCCACCUACUA |
| | | | UCCCGGCAAUAGAGCGGAGUUCGUCUGGUUUGAGG |
| | | | ACGGCCGUAGGGUGUUCGAUCCUGCCCAGAUUCAC |
| | | | ACCCAGACACAGGAGAAUCCCGACGGCUUUAGCAC |
| | | | AGUGAGCACUGUGACGUCUGCUGCCGUUGGUGGUC |
| | | | AAGGGCCUCCUCGUACCUUCACAUGCCAAUUGACC |
| | | | UGGCACCGCGACUCAGUUAGCUUUAGCCGCCGGAA |
| | | | UGCCAGUGGGACCGCCAGUGUUCUCCCAAGGCCGA |
| | | | CAAUCACCAUGGAGUUCACUGGCGACCAUGCAGUG |
| | | | UGCACAGCUGGGUGUGUCCCAGAAGGCGUGACUUU |
| | | | CGCCUGGUUUCUGGGUGAUGACUCCUCACCCGCCG |
| | | | AGAAAGUAGCUGUCGCUUCCCAGACUUCCUGUGGA |
| | | | CGUCCUGGAACUGCGACAAUCCGAAGCACACUGCC |
| | | | GGUUUCCUACGAGCAGACGGAGUACAUAUGCCGCC |
| | | | UUGCAGGCUACCCCGAUGGAAUUCCAGUCCUUGAG |
| | | | CACCAUUGA |
| 278 | HSV-2 gC | Version | AGUCCAGGAAGGACGAUUACGGUGGGACCCAGAGG |
| | [UL44] | 4.1 | UAAUGCGUCCAAUGCUGCGCCAUCCGCUUCUCCAC |
| | | | GGAACGCAUCUGCACCCAGGACUACACCGACACCA |
| | | | CCUCAGCCGCGCAAAGCCACCAAGAGCAAGGCCAG |
| | | | CACAGCCAAACCCGCUCCUCCACCUAAAACCGGAC |
| | | | CACCUAAGACCAGCUCUGAACCCGUCAGAUGCAAC |
| | | | AGGCACGAUCCGUUGGCCAGAUAUGGCAGUCGCGU |
| | | | CCAGAUCAGGUGUCGCUUCCCUAACAGCACACGGA |
| | | | CCGAGUUCAGGCUGCAAAUUUGGCGCUACGCUACA |
| | | | GCCACUGACGCAGAGAUUGGCACUGCUCCCAGUCU |
| | | | GGAGGAGGUCAUGGUGAACGUGUCUGCUCCACCAG |
| | | | GCGGUCAGCUGGUCUAUGACUCAGCCCCUAAUCGC |
| | | | ACAGAUCCUCACGUGAUUUGGGCAGAAGGUGCGGG |
| | | | GCCUGGGGCCUCCCCAAGGCUCUACUCAGUGGUUG |
| | | | GACCCCUUGGGAGACAGCGGCUGAUCAUCGAGGAA |
| | | | CUGACUCUCGAAACCCAAGGUAUGUACUACUGGGU |
| | | | AUGGGGCAGAACAGACAGACCUUCAGCUUAUGGCA |
| | | | CCUGGGUGCGGGUGAGAGUGUUUAGGCCUCCCUCC |
| | | | CUGACGAUCCAUCCCCAUGCUGUGCUGGAAGGACA |
| | | | GCCGUUCAAGGCAACAUGCACAGCAGCCACUUACU |
| | | | AUCCCGGAAACCGUGCUGAGUUUGUGUGGUUCGAG |
| | | | GAUGGGCGACGUGUAUUCGACCCUGCCCAGAUUCA |
| | | | CACCCAGACACAGGAGAAUCCCGACGGGUUUUCCA |
| | | | CUGUGAGCACCGUGACAUCAGCGGCAGUAGGAGGG |
| | | | CAGGGCCCACCCCGAACGUUCACUUGCCAGCUUAC |
| | | | UUGGCAUCGGGACAGUGUUAGCUUUAGCCGCCGGA |
| | | | AUGCCUCUGGCACCGCAUCCGUCCUUCCUCGCCCA |
| | | | ACCAUCACCAUGGAAUUCACUGGCGAUCACGCCGU |
| | | | UUGUACAGCCGGGUGUGUUCCCGAGGGAGUGACCU |
| | | | UUGCUUGGUUUCUGGGCGAUGACUCAAGCCCAGCC |
| | | | GAAAAGGUGGCCGUCGCCUCCCAAACGAGCUGUGG |
| | | | GCGACCUGGCACCGCUACCAUACGUAGCACUCUGC |
| | | | CCGUUUCCUACGAACAGACCGAGUAUAUCUGCCGA |
| | | | UUGGCCGGUUACCCCGAUGGGAUACCAGUCCUGGA |
| | | | GCACCACUGA |
| 279 | HSV-2 gC | Version | UCCCCGGGUCGAACAAUCACUGUUGGGCCCAGGGG |
| | [UL44] | 4.2 | AAAUGCCAGCAAUGCUGCACCUUCAGCAAGCCCAC |
| | | | GAAACGCUUCAGCACCCAGGACAACACCCACUCCA |
| | | | CCUCAACCGCGGAAAGCCACCAAGAGCAAGGCAAG |
| | | | UACCGCCAAACCCGCUCCUCCUCCCAAGACAGGGC |
| | | | CACCCAAGACCUCUAGUGAGCCAGUGAGGUGUAAC |
| | | | CGCCAUGAUCCCCUUGCCAGAUACGGGAGCAGAGU |
| | | | GCAGAUUAGGUGCCGGUUUCCAAACUCCACGAGAA |
| | | | CCGAAUUUCGCCUCCAGAUUUGGCGGUAUGCGACU |
| | | | GCCACAGACGCAGAGAUUGGUACCGCUCCCAGCCU |
| | | | GGAGGAGGUCAUGGUGAACGUGUCAGCGCCUCCGG |
| | | | GUGGCCAGCUGGUCUACGACUCUGCCCCAAAUCGA |
| | | | ACCGACCCUCACGUCAUCUGGGCUGAAGGAGCGGG |
| | | | ACCAGGAGCCUCUCCACGCUUGUAUAGCGUAGUUG |
| | | | GCCCUCUGGGGAGACAGCGCCUGAUCAUUGAGGAA |
| | | | CUGACCCUUGAGACACAGGGGAUGUACUACUGGGU |
| | | | GUGGGGCAGGACUGACAGGCCCAGUGCCUAUGGAA |
| | | | CUUGGGUUAGGGUCCGCGUCUUUCGGCCACCCAGU |
| | | | CUGACCAUCCAUCCACAUGCCGUGCUGGAAGGCCA |
| | | | GCCCUUCAAAGCGACUUGCACUGCCGCCACGUACU |
| | | | AUCCAGGGAAUAGAGCCGAGUUCGUUUGGUUCGAG |
| | | | GAUGGCCGGAGAGUAUUCGAUCCAGCUCAGAUCCA |
| | | | CACCCAGACGCAGGAAAACCCGGACGGCUUUAGCA |
| | | | CGGUGAGUACCGUCACCUCUGCUGCCGUCGGAGGC |
| | | | CAAGGACCUCCCCGUACCUUCACAUGCCAGCUUAC |
| | | | AUGGCACCGGGACUCAGUAAGCUUUUCACGUCGUA |
| | | | AUGCAUCCGGUACUGCUUCUGUGCUGCCUCGACCC |
| | | | ACCAUCACCAUGGAGUUCACAGGGGAUCACGCAGU |
| | | | GUGUACGGCAGGCUGCGUGCCUGAAGGCGUGACAU |
| | | | UCGCCUGGUUUCUCGGUGAUGACUCCUCUCCUGCU |
| | | | GAAAAGGUGGCUGUAGCCUCCCAAACAAGCUGUGG |
| | | | UCGGCCUGGAACUGCCACUAUACGCUCCACUCUCC |
| | | | CGGUGUCCUACGAACAGACCGAGUACAUAUGCAGA |
| | | | CUGGCUGGAUAUCCCGAUGGCAUUCCCGUGCUGGA |
| | | | GCAUCACUGA |
| 280 | HSV-2 gC | Version | UCUCCAGGCAGAACUAUCACAGUGGGACCCAGAGG |
| | [UL44] | 4.3 | GAAUGCCAGCAAUGCAGCCCCGAGUGCCAGCCCUC |
| | | | GUAACGCCAGCGCUCCCAGAACAACCCCAACUCCA |
| | | | CCGCAGCCUAGAAAGGCGACCAAGUCCAAAGCAUC |
| | | | CACUGCAAAACCAGCCCCACCUCCCAAAACGGGAC |
| | | | CUCCCAAGACCAGCUCCGAGCCUGUAAGGUGCAAU |
| | | | CGGCAUGACCCCUUGGCCCGAUAUGGCAGUCGCGU |
| | | | GCAGAUUCGAUGUCGGUUUCCCAACUCUACCCGGA |
| | | | CUGAGUUCCGGUUGCAGAUCUGGAGGUAUGCGACC |
| | | | GCCACUGACGCUGAGAUCGGCACAGCACCAAGCCU |
| | | | GGAAGAAGUGAUGGUGAACGUUAGUGCUCCUCCGG |
| | | | GCGGGCAACUCGUGUAUGACUCCGCACCCAACCGC |
| | | | ACAGAUCCUCACGUGAUUUGGGCCGAAGGAGCCGG |
| | | | ACCCGGUGCGUCACCUAGGCUCUACUCUGUCGUAG |
| | | | GACCACUGGGCCGUCAACGCCUGAUAAUCGAGGAG |
| | | | CUGACUCUGGAGACACAGGGUAUGUACUACUGGGU |
| | | | CUGGGGCAGAACCGACAGGCCAUCUGCUUACGGGA |
| | | | CAUGGGUCCGCGUUCGAGUAUUUCGGCCACCCUCA |
| | | | CUGACCAUACAUCCCCAUGCCGUUCUUGAAGGGCA |
| | | | GCCUUUCAAGGCAACCUGUACUGCUGCCACAUACU |
| | | | AUCCCGGGAAUAGGGCCGAGUUCGUCUGGUUUGAA |
| | | | GAUGGCCGAAGGGUGUUUGACCCGGCUCAGAUCCA |
| | | | CACCCAGACACAGGAGAACCCCGAUGGCUUCAGUA |
| | | | CGGUGUCUACCGUCACAAGCGCCGCUGUGGGUGGC |
| | | | CAAGGUCCUCCCAGAACUUUCACCUGUCAGCUGAC |
| | | | GUGGCACAGGGAUUCCGUGAGCUUUUCCCGCCGCA |
| | | | AUGCGUCAGGGACCGCCUCCGUGCUUCCUCGGCCA |
| | | | ACCAUCACAAUGGAAUUCACGGGUGAUCACGCUGU |
| | | | CUGCACAGCUGGCUGCGUUCCUGAGGGCGUGACAU |
| | | | UCGCAUGGUUUCUUGGUGACGACUCAUCUCCCGCA |
| | | | GAGAAGGUGGCUGUUGCCUCACAAACGAGUUGUGG |
| | | | GCGUCCAGGCACUGCCACCAUUCGGUCCACCCUCC |
| | | | CCGUAAGCUACGAACAGACUGAGUAUAUUUGCAGA |
| | | | CUGGCUGGAUACCCGGAUGGGAUUCCUGUCCUGGA |
| | | | ACAUCACUGA |
| 281 | HSV-2 gC | Version | UCUCCCGGACGAACUAUCACUGUAGGCCCAAGGGG |
| | [UL44] | 4.4 | CAACGCUAGUAAUGCCGCUCCCAGUGCUUCACCAC |
| | | | GCAAUGCGAGCGCACCCAGAACUACACCCACACCU |
| | | | CCUCAGCCGAGGAAAGCCACCAAGUCCAAAGCCAG |
| | | | CACCGCCAAACCCGCUCCUCCACCUAAAACAGGGC |
| | | | CUCCCAAGACCUCAAGCGAGCCCGUUAGAUGCAAU |
| | | | CGGCAUGAUCCACUGGCUCGUUACGGUUCUCGGGU |
| | | | CCAGAUACGCUGUAGGUUUCCUAACUCCACACGAA |
| | | | CCGAGUUCAGAUUGCAGAUCUGGAGAUAUGCCACA |
| | | | GCCACUGACGCUGAGAUUGGCACUGCACCUAGUCU |
| | | | GGAGGAGGUGAUGGUGAACGUGAGCGCCCCUCCUG |
| | | | GCGGUCAGCUUGUGUAUGACUCAGCACCGAAUCGC |
| | | | ACAGAUCCGCACGUCAUAUGGGCCGAAGGUGCAGG |
| | | | GCCCGGUGCAUCCCCUCGGCUGUAUUCCGUGGUCG |
| | | | GACCACUCGGGCGCCAGAGGCUUAUCAUUGAGGAA |
| | | | CUGACCCUCGAAACCCAGGGUAUGUACUACUGGGU |
| | | | AUGGGGCCGUACCGACCGGCCUAGCGCCUACGGAA |
| | | | CUUGGGUGAGAGUUCGGGUGUUCAGACCGCCAAGU |
| | | | CUUACAAUUCACCCUCAUGCCGUGCUCGAAGGUCA |
| | | | GCCAUUUAAGGCCACGUGUACUGCGGCUACGUACU |
| | | | AUCCCGGGAAUCGGGCUGAAUUCGUGUGGUUUGAG |
| | | | GAUGGCAGGAGAGUGUUCGACCCAGCCCAAAUCCA |
| | | | CACCCAAACACAGGAGAACCCAGACGGGUUUUCCA |
| | | | CCGUGUCAACGGUCACAUCUGCCGCCGUCGGAGGA |
| | | | CAAGGGCCACCCAGAACCUUCACAUGCCAGCUGAC |
| | | | CUGGCAUAGGGAUAGCGUAAGCUUUAGCCGGCGAA |
| | | | ACGCAUCUGGAACGGCGAGCGUUCUGCCUCGACCA |
| | | | ACAAUCACCAUGGAGUUCACCGGCGAUCACGCAGU |
| | | | GUGCACUGCUGGGUGUGUACCCGAAGGCGUGACAU |
| | | | UUGCCUGGUUUCUGGGAGAUGACUCCUCACCCGCA |
| | | | GAAAAGGUCGCCGUUGCAUCUCAGACCAGUUGUGG |
| | | | CAGGCCCGGGACUGCUACCAUCCGCAGCACUCUGC |
| | | | CGGUGUCUUACGAACAGACGGAGUACAUUUGCCGC |
| | | | UUGGCGGGCUAUCCAGACGGCAUUCCAGUUCUGGA |
| | | | GCAUCACUGA |
| 20 | HSV-2 gD | | AAGUACGCCCUGGCCGACCCCUCCCUGAAGAUGGC |
| | [US6] | | CGACCCCAACCGCUUCCGCGGCAAGAACCUGCCCG |
| | | | UGCUGGACCAGCUGACCGACCCCCCCGGCGUGAAG |
| | | | CGCGUGUACCACAUCCAGCCCUCCCUGGAGGACCC |
| | | | CUUCCAGCCCCCCUCCAUCCCCAUCACCGUGUACUA |
| | | | CGCCGUGCUGGAGCGCGCCUGCCGCUCCGUGCUGC |
| | | | UGCACGCCCCCUCCGAGGCCCCCCAGAUCGUGCGC |
| | | | GGCGCCUCCGACGAGGCCCGCAAGCACACCUACAA |
| | | | CCUGACCAUCGCCUGGUACCGCAUGGGCGACAACU |
| | | | GCGCCAUCCCCAUCACCGUGAUGGAGUACACCGAG |
| | | | UGCCCCUACAACAAGUCCCUGGGCGUGUGCCCCAU |
| | | | CCGCACCCAGCCCCGCUGGUCCUACUACGACUCCU |
| | | | UCUCCGCCGUGUCCGAGGACAACCUGGGCUUCCUG |
| | | | AUGCACGCCCCCGCCUUCGAGACCGCCGGCACCUA |
| | | | CCUGCGCCUGGUGAAGAUCAACGACUGGACCGAGA |
| | | | UCACCCAGUUCAUCCUGGAGCACCGCGCCCGCGCC |
| | | | UCCUGCAAGUACGCCCUGCCCCUGCGCAUCCCCCCC |
| | | | GCCGCCUGCCUGACCUCCAAGGCCUACCAGCAGGG |
| | | | CGUGACCGUGGACUCCAUCGGCAUGCUGCCCCGCU |
| | | | UCAUCCCCGAGAACCAGCGCACCGUGGCCCUGUAC |
| | | | UCCCUGAAGAUCGCCGGCUGGCACGGCCCCAAGCC |
| | | | CCCCUACACCUCCACCCUGCUGCCCCCCGAGCUGUC |
| | | | CGACACCACCAACGCCACCCAGCCCGAGCUGGUGC |
| | | | CCGAGGACCCCGAGGACUCCGCCCUGCUGGAGGAC |
| | | | CCCGCCGGCACCGUGUCCUCCCAGAUCCCCCCCAAC |
| | | | UGGCACAUCCCCUCCAUCCAGGACGUGGCCCCCCA |
| | | | CCACUAA |
| 21 | HSV-2 gD | Version 1 | AAAUACGCCCUGGCCGAUCCUAGCCUGAAGAUGGC |
| | [US6] | | UGACCCCAACCGGUUCCGGGGCAAGAAUCUGCCUG |
| | | | UUCUGGACCAGCUGACCGAUCCUCCUGGCGUGAAA |
| | | | CGGGUGUACCACAUCCAGCCAAGCCUGGAAGAUCC |
| | | | CUUCCAGCCUCCUAGCAUCCCCAUCACCGUGUACU |
| | | | ACGCCGUGCUGGAAAGGGCCUGUAGAAGCGUGCUG |
| | | | CUGCACGCCCCAUCUGAAGCCCCUCAAAUCGUCAG |
| | | | AGGCGCUUCCGACGAGGCCAGAAAGCACACCUACA |
| | | | ACCUGACAAUCGCCUGGUACAGAAUGGGCGACAAC |
| | | | UGCGCCAUUCCUAUCACCGUGAUGGAGUACACCGA |
| | | | GUGUCCCUACAACAAGAGCCUGGGCGUGUGCCCCA |
| | | | UCAGAACACAGCCUAGAUGGUCCUACUACGACAGC |
| | | | UUCAGCGCCGUGUCCGAGGACAAUCUGGGCUUCCU |
| | | | GAUGCAUGCCCCUGCCUUUGAGACAGCCGGCACCU |
| | | | AUCUGCGGCUGGUCAAGAUCAACGACUGGACCGAG |
| | | | AUCACCCAGUUCAUCCUGGAACACAGAGCCAGAGC |
| | | | CAGCUGCAAAUACGCUCUGCCCCUGAGAAUUCCUC |
| | | | CUGCCGCCUGUCUGACAAGCAAGGCCUAUCAGCAG |
| | | | GGCGUGACCGUGGAUAGCAUCGGCAUGCUGCCCAG |
| | | | AUUCAUCCCCGAGAACCAGAGAACAGUGGCCCUGU |
| | | | ACUCCCUGAAGAUCGCCGGAUGGCACGGACCCAAG |
| | | | CCUCCAUACACAAGCACACUGCUGCCUCCAGAGCU |
| | | | GAGCGACACCACCAAUGCCACACAGCCUGAACUGG |
| | | | UGCCUGAGGACCCAGAGGAUUCUGCCCUGCUUGAA |
| | | | GAUCCUGCCGGCACCGUGUCUAGCCAGAUUCCUCC |
| | | | UAACUGGCACAUCCCCAGCAUCCAGGAUGUGGCCC |
| | | | CUCAUCAUUGA |
| 22 | HSV-2 gD | Version 2 | AAAUAUGCUCUCGCUGAUCCGAGCCUCAAGAUGGC |
| | [US6] | | AGAUCCCAACCGAUUUCGGGGAAAGAAUCUGCCAG |
| | | | UACUGGACCAGCUGACGGACCCACCUGGCGUCAAA |
| | | | CGCGUCUACCACAUACAGCCUAGUCUUGAGGACCC |
| | | | UUUUCAGCCACCGUCUAUCCCCAUUACCGUGUACU |
| | | | AUGCCGUGCUGGAACGCGCGUGUAGGUCAGUUCUG |
| | | | CUGCAUGCCCCAUCCGAAGCCCCCCAGAUCGUCAG |
| | | | AGGAGCUUCUGAUGAAGCACGCAAACACACCUACA |
| | | | ACCUCACAAUAGCGUGGUAUCGAAUGGGCGAUAAC |
| | | | UGCGCAAUUCCCAUCACAGUCAUGGAGUACACGGA |
| | | | GUGCCCCUACAACAAGAGCCUCGGUGUUUGCCCUA |
| | | | UCAGGACACAACCCAGGUGGAGCUAUUACGACAGU |
| | | | UUCAGCGCCGUGUCUGAGGACAAUCUGGGGUUUCU |
| | | | GAUGCACGCACCCGCCUUCGAGACUGCCGGCACCU |
| | | | ACUUGCGGCUGGUGAAGAUCAACGACUGGACUGAG |
| | | | AUCACCCAGUUCAUCCUGGAACAUAGGGCCAGAGC |
| | | | CAGCUGCAAGUAUGCUCUUCCCCUGCGGAUUCCGC |
| | | | CUGCAGCAUGUCUGACCUCAAAAGCCUACCAGCAA |
| | | | GGGGUGACUGUGGACAGCAUUGGCAUGCUGCCUCG |
| | | | UUUCAUUCCCGAGAAUCAACGGACAGUGGCUCUGU |
| | | | AUUCCCUGAAGAUCGCAGGAUGGCAUGGGCCCAAA |
| | | | CCACCUUAUACCUCUACGUUGCUUCCACCAGAACU |
| | | | CAGUGACACCACUAAUGCGACACAGCCAGAACUUG |
| | | | UGCCUGAGGAUCCUGAAGAUAGCGCUCUGUUGGAG |
| | | | GAUCCAGCCGGUACUGUGUCCUCCCAGAUACCACC |
| | | | CAAUUGGCACAUUCCUUCCAUUCAGGACGUAGCUC |
| | | | CGCAUCACUGA |
| 286 | HSV-2 gD | Version | AAAUAUGCUCUCGCUGAUCCGAGCCUCAAGAUGGC |
| | [US6] | 2.1 | AGAUCCCAACCGAUUUCGGGGAAAGAAUCUGCCAG |
| | | | UACUGGACCAGCUGACGGACCCACCUGGCGUCAAA |
| | | | CGCGUCUACCACAUACAGCCUAGUCUUGAGGACCC |
| | | | UUUUCAGCCACCGUCUAUCCCCAUUACCGUGUACU |
| | | | AUGCCGUGCUGGAACGCGCGUGUAGGUCAGUUCUG |
| | | | CUGCAUGCCCCAUCCGAAGCCCCCCAGAUCGUCAG |
| | | | AGGAGCUUCUGAUGAAGCACGCAAACACACCUACA |
| | | | ACCUCACAAUAGCGUGGUAUCGAAUGGGCGAUAAC |
| | | | UGCGCAAUUCCCAUCACAGUCAUGGAGUACACGGA |
| | | | GUGCCCCUACAACAAGAGCCUCGGUGUUUGCCCUA |
| | | | UCAGGACACAACCCAGGUGGAGCUAUUACGACAGU |
| | | | UUCAGCGCCGUGUCUGAGGACAAUCUGGGGUUUCU |
| | | | GAUGCACGCACCCGCCUUCGAGACUGCCGGCACCU |
| | | | ACUUGCGGCUGGUGAAGAUCAACGACUGGACUGAG |
| | | | AUCACCCAGUUCAUCCUGGAACAUAGGGCCAGAGC |
| | | | CAGCUGCAAGUAUGCCCUUCCCCUGCGGAUUCCGC |
| | | | CUGCAGCAUGUCUGACCUCAAAAGCCUACCAGCAA |
| | | | GGGGUGACUGUGGACAGCAUUGGCAUGCUGCCUCG |
| | | | UUUCAUUCCCGAGAAUCAACGGACAGUGGCUCUGU |
| | | | AUUCCCUGAAGAUCGCAGGAUGGCAUGGGCCCAAA |
| | | | CCACCUUAUACCUCUACGUUGCUUCCACCAGAACU |
| | | | CAGUGACACCACUAAUGCGACACAGCCAGAACUUG |
| | | | UGCCUGAGGAUCCUGAAGAUAGCGCUCUGUUGGAG |
| | | | GAUCCAGCCGGUACUGUGUCCUCCCAGAUACCACC |
| | | | CAAUUGGCACAUUCCUUCCAUUCAGGACGUAGCUC |
| | | | CGCAUCACUGA |
| 340 | HSV-2 gD | Version | AAAUAUGCUCUCGCUGAUCCGAGCCUCAAGAUGGC |
| | [US6] | 2.2 | AGAUCCCAACCGAUUUCGGGGAAAGAAUCUGCCAG |
| | | | UACUGGACCAGCUGACGGACCCACCUGGCGUCAAA |
| | | | CGCGUCUACCACAUACAGCCUAGUCUUGAGGACCC |
| | | | UUUUCAGCCACCGUCUAUCCCCAUUACCGUGUACU |
| | | | AUGCCGUGCUGGAACGCGCGUGUAGGUCAGUUCUG |
| | | | CUGCAUGCCCCAUCCGAAGCCCCCCAGAUCGUCAG |
| | | | AGGAGCUUCUGAUGAAGCACGCAAACACACCUACA |
| | | | ACCUCACAAUAGCGUGGUAUCGAAUGGGCGAUAAC |
| | | | UGCGCAAUUCCCAUCACAGUCAUGGAGUACACGGA |
| | | | GUGCCCCUACAACAAGAGCCUCGGUGUUUGCCCUA |
| | | | UCAGGACACAACCCAGGUGGAGCUAUUACGACAGU |
| | | | UUCAGCGCCGUGUCUGAGGACAAUCUGGGGUUUCU |
| | | | GAUGCACGCACCCGCCUUCGAGACUGCCGGCACCU |
| | | | ACUUGCGGCUGGUGAAGAUCAACGACUGGACUGAG |
| | | | AUCACCCAGUUCAUCCUGGAACAUAGGGCCAGAGC |
| | | | CAGCUGCAAGUAUGCCCUUCCCCUGCGGAUUCCGC |
| | | | CUGCAGCAUGUCUGACCUCAAAAGCCUACCAGCAA |
| | | | GGGGUGACUGUGGACAGCAUUGGCAUGCUGCCUCG |
| | | | UUUCAUUCCCGAGAAUCAACGGACAGUGGCUCUGU |
| | | | AUUCCCUGAAGAUCGCAGGAUGGCAUGGGCCCAAA |
| | | | CCACCUUAUACCUCUACGUUGCUUCCACCAGAACU |
| | | | CAGUGACACCACUAAUGCGACACAGCCAGAACUUG |
| | | | UGCCUGAGGAUCCUGAAGAUAGCGCUCUGUUGGAG |
| | | | GAUCCAGCCGGUACUGUGUCCUCCCAGAUACCACC |
| | | | CAAUUGGCACAUUCCUUCCAUUCAGGACGUAGCUC |
| | | | CGCAUCACUGAUAA |
| 23 | HSV-2 gD | Version 3 | AAAUACGCCCUGGCCGAUCCCUCCCUGAAAAUGGC |
| | [US6] | | CGAUCCCAACAGGUUCAGAGGCAAAAACCUGCCCG |
| | | | UGCUGGAUCAGCUGACCGAUCCCCCUGGCGUGAAA |
| | | | AGAGUGUACCACAUCCAGCCCUCCCUGGAAGAUCC |
| | | | CUUCCAGCCCCCUUCCAUCCCCAUCACCGUGUACU |
| | | | ACGCCGUGCUGGAAAGAGCUUGCAGAUCCGUGCUG |
| | | | CUGCACGCCCCCUCCGAAGCCCCUCAGAUCGUGAG |
| | | | AGGCGCCUCCGAUGAAGCCAGAAAACACACCUACA |
| | | | ACCUGACCAUCGCCUGGUACAGAAUGGGCGAUAAC |
| | | | UGCGCCAUCCCCAUCACCGUGAUGGAAUACACCGA |
| | | | AUGCCCCUACAACAAAUCCCUGGGCGUGUGCCCCA |
| | | | UCAGAACCCAGCCCAGAUGGUCCUACUACGAUUCC |
| | | | UUCUCCGCCGUGUCCGAAGAUAACCUGGGCUUCCU |
| | | | GAUGCACGCCCCCGCCUUCGAAACCGCCGGCACCU |
| | | | ACCUGAGACUGGUGAAAAUCAACGAUUGGACCGAA |
| | | | AUCACCCAGUUCAUCCUGGAACACAGAGCCAGAGC |
| | | | CUCCUGCAAAUACGCCCUGCCCCUGAGAAUCCCUC |
| | | | CCGCCGCCUGCCUGACCUCCAAAGCCUACCAGCAG |
| | | | GGCGUGACCGUGGAUUCCAUCGGCAUGCUGCCCAG |
| | | | AUUCAUCCCCGAAAACCAGAGAACCGUGGCCCUGU |
| | | | ACUCCCUGAAAAUCGCCGGCUGGCACGGCCCCAAA |
| | | | CCCCCUUACACCUCCACCCUGCUGCCCCCUGAACUG |
| | | | UCCGAUACCACCAACGCCACCCAGCCCGAACUGGU |
| | | | GCCCGAAGAUCCCGAAGAUUCCGCCCUGCUGGAAG |
| | | | AUCCCGCCGGCACCGUGUCCUCCCAGAUCCCUCCCA |
| | | | ACUGGCACAUCCCCUCCAUCCAGGAUGUGGCCCCU |
| | | | CACCACUGA |
| 143 | HSV-2 gD | Version 2 | GAUCCGAGCCUCAAGAUGGCAGAUCCCAACCGAUU |
| | (US6) | EAM | UCGGGGAAAGAAUCUGCCAGUACUGGACCAGCUGA |
| | | | CGGACCCACCUGGCGUCAAACGCGUCUACCACAUA |
| | | | CAGCCUAGUCUUGAGGACCCUUUUCAGCCACCGUC |
| | | | UAUCCCCAUUACCGUGUACUAUGCCGUGCUGGAAC |
| | | | GCGCGUGUAGGUCAGUUCUGCUGCAUGCCCCAUCC |
| | | | GAAGCCCCCCAGAUCGUCAGAGGAGCUUCUGAUGA |
| | | | AGCACGCAAACACACCUACAACCUCACAAUAGCGU |
| | | | GGUAUCGAAUGGGCGAUAACUGCGCAAUUCCCAUC |
| | | | ACAGUCAUGGAGUACACGGAGUGCCCCUACAACAA |
| | | | GAGCCUCGGUGUUUGCCCUAUCAGGACACAACCCA |
| | | | GGUGGAGCUAUUACGACAGUUUCAGCGCCGUGUCU |
| | | | GAGGACAAUCUGGGGUUUCUGAUGCACGCACCCGC |
| | | | CUUCGAGACUGCCGGCACCUACUUGCGGCUGGUGA |
| | | | AGAUCAACGACUGGACUGAGAUCACCCAGUUCAUC |
| | | | CUGGAACAUAGGGCCAGAGCCAGCUGCAAGUAUGC |
| | | | CCUUCCCCUGCGGAUUCCGCCUGCAGCAUGUCUGA |
| | | | CCUCAAAAGCCUACCAGCAAGGGGUGACUGUGGAC |
| | | | AGCAUUGGCAUGCUGCCUCGUUUCAUUCCCGAGAA |
| | | | UCAACGGACAGUGGCUCUGUAUUCCCUGAAGAUCG |
| | | | CAGGAUGGCAUGGGCCCAAACCACCUUAUACCUCU |
| | | | ACGUUGCUUCCACCAGAACUCAGUGACACCACUAA |
| | | | UGCGACACAGCCAGAACUUGUGCCUGAGGAUCCUG |
| | | | AAGAUAGCGCUCUGUUGGAGGAUCCAGCCGGUACU |
| | | | GUGUCCUCCCAGAUACCACCCAAUUGGCACAUUCC |
| | | | UUCCAUUCAGGACGUAGCUCCGCAUCACUGA |
| 24 | HSV-2 gE | | CGCACCUCCUGGAAGCGCGUGACCUCCGGCGAGGA |
| | [US8] | | CGUGGUGCUGCUGCCCGCCCCCGCCGGCCCCGAGG |
| | | | AGCGCACCCGCGCCCACAAGCUGCUGUGGGCCGCC |
| | | | GAGCCCCUGGACGCCUGCGGCCCCCUGCGCCCCUCC |
| | | | UGGGUGGCCCUGUGGCCCCCCCGCCGCGUGCUGGA |
| | | | GACCGUGGUGGACGCCGCCUGCAUGCGCGCCCCCG |
| | | | AGCCCCUGGCCAUCGCCUACUCCCCCCCCUUCCCCG |
| | | | CCGGCGACGAGGGCCUGUACUCCGAGCUGGCCUGG |
| | | | CGCGACCGCGUGGCCGUGGUGAACGAGUCCCUGGU |
| | | | GAUCUACGGCGCCCUGGAGACCGACUCCGGCCUGU |
| | | | ACACCCUGUCCGUGGUGGGCCUGUCCGACGAGGCC |
| | | | CGCCAGGUGGCCUCCGUGGUGCUGGUGGUGGAGCC |
| | | | CGCCCCCGUGCCCACCCCCACCCCCGACGACUACGA |
| | | | CGAGGAGGACGACGCCGGCGUGUCCGAGCGCACCC |
| | | | CCGUGUCCGUGCCCCCCCCCACCCCCCCCCGCCGCC |
| | | | CCCCCGUGGCCCCCCCCACCCACCCCCGCGUGAUCC |
| | | | CCGAGGUGUCCCACGUGCGCGGCGUGACCGUGCAC |
| | | | AUGGAGACCCCCGAGGCCAUCCUGUUCGCCCCCGG |
| | | | CGAGACCUUCGGCACCAACGUGUCCAUCCACGCCA |
| | | | UCGCCCACGACGACGGCCCCUACGCCAUGGACGUG |
| | | | GUGUGGAUGCGCUUCGACGUGCCCUCCUCCUGCGC |
| | | | CGAGAUGCGCAUCUACGAGGCCUGCCUGUACCACC |
| | | | CCCAGCUGCCCGAGUGCCUGUCCCCCGCCGACGCCC |
| | | | CCUGCGCCGUGUCCUCCUGGGCCUACCGCCUGGCC |
| | | | GUGCGCUCCUACGCCGGCUGCUCCCGCACCACCCCC |
| | | | CCCCCCCGCUGCUUCGCCGAGGCCCGCAUGGAGCCC |
| | | | GUGCCCGGCCUGGCCUGGCUGGCCUCCACCGUGAA |
| | | | CCUGGAGUUCCAGCACGCCUCCCCCCAGCACGCCG |
| | | | GCCUGUACCUGUGCGUGGUGUACGUGGACGACCAC |
| | | | AUCCACGCCUGGGGCCACAUGACCAUCUCCACCGC |
| | | | CGCCCAGUACCGCAACGCCGUGGUGGAGCAGCACC |
| | | | UGCCCCAGCGCCAGCCCGAGCCCGUGGAGCCCACCC |
| | | | GCCCCCACGUGCGCGCCUAA |
| 25 | HSV-2 gE | Version 1 | AGAACCAGCUGGAAAAGAGUGACCAGCGGCGAGGA |
| | [US8] | | UGUGGUGCUGCUUCCUGCUCCUGCUGGCCCCGAGG |
| | | | AAAGAACAAGAGCCCACAAACUGCUGUGGGCCGCU |
| | | | GAGCCUCUUGAUGCCUGUGGACCUCUCAGACCUAG |
| | | | CUGGGUUGCACUGUGGCCACCUCGGAGAGUGCUGG |
| | | | AAACAGUGGUGGAUGCCGCCUGCAUGAGAGCCCCU |
| | | | GAACCUCUGGCCAUUGCCUACUCUCCACCAUUUCC |
| | | | AGCCGGCGACGAGGGCCUGUAUUCUGAGCUUGCUU |
| | | | GGAGAGACAGAGUGGCCGUGGUCAACGAGAGCCUG |
| | | | GUUAUCUAUGGCGCCCUGGAAACCGACAGCGGCCU |
| | | | GUACACACUGUCUGUCGUGGGCCUGUCUGACGAGG |
| | | | CUAGACAGGUGGCAUCUGUGGUCCUGGUGGUGGAA |
| | | | CCUGCUCCAGUGCCUACACCUACACCUGACGACUA |
| | | | CGACGAGGAAGAUGACGCUGGCGUCAGCGAGAGAA |
| | | | CCCCUGUUUCUGUGCCUCCUCCUACGCCUCCUCGU |
| | | | AGACCUCCUGUUGCUCCUCCAACACACCCCAGAGU |
| | | | GAUCCCUGAAGUGUCUCACGUGCGGGGCGUGACCG |
| | | | UGCACAUGGAAACACCUGAGGCCAUCCUGUUCGCC |
| | | | CCUGGCGAGACAUUUGGCACCAACGUGUCCAUCCA |
| | | | CGCUAUCGCCCACGACGAUGGCCCUUACGCCAUGG |
| | | | AUGUCGUGUGGAUGAGAUUCGACGUGCCCAGCAGC |
| | | | UGUGCCGAGAUGAGAAUCUAUGAGGCCUGCCUGUA |
| | | | UCACCCUCAGCUGCCCGAAUGUCUGAGCCCUGCUG |
| | | | AUGCCCCUUGUGCCGUUAGCAGCUGGGCCUAUAGA |
| | | | CUGGCCGUGCGGUCUUAUGCCGGCUGCUCUAGAAC |
| | | | AACCCCUCCUCCUCGGUGUUUCGCCGAGGCCAGAA |
| | | | UGGAACCUGUUCCUGGACUGGCCUGGCUGGCCUCC |
| | | | ACAGUGAACCUGGAAUUUCAGCACGCCUCUCCACA |
| | | | GCACGCCGGCCUGUAUCUGUGUGUGGUGUACGUGG |
| | | | ACGAUCACAUCCACGCCUGGGGCCACAUGACCAUC |
| | | | UCUACAGCCGCUCAGUACCGGAACGCCGUGGUUGA |
| | | | ACAGCAUCUGCCUCAGAGACAGCCCGAGCCUGUGG |
| | | | AACCUACAAGACCUCAUGUUCGGGCCUGA |
| 282 | HSV-2 gE | Version | AGAACCUCUUGGAAGCGCGUGACAAGCGGCGAGGA |
| | [US8] | 1.1 | UGUGGUUCUGCUUCCUGCUCCUGCCGGACCUGAGG |
| | | | AAAGAACAAGAGCCCACAAGCUGCUGUGGGCCGCU |
| | | | GAACCUUUGGAUGCCUGUGGACCUCUGAGGCCUUC |
| | | | UUGGGUUGCACUGUGGCCACCUCGGAGAGUGCUGG |
| | | | AAACAGUGGUGGAUGCCGCCUGCAUGAGAGCCCCU |
| | | | GAACCUCUGGCCAUUGCCUACUCUCCACCUUUUCC |
| | | | AGCCGGCGACGAGGGCCUGUAUUCUGAGCUUGCUU |
| | | | GGAGAGACAGAGUGGCCGUGGUCAACGAGAGCCUG |
| | | | GUUAUCUAUGGCGCCCUGGAAACCGACAGCGGCCU |
| | | | GUACACACUGUCUGUCGUGGGCCUGUCUGACGAGG |
| | | | CUAGACAGGUGGCAUCUGUGGUGCUGGUGGUGGA |
| | | | ACCUGCUCCAGUGCCUACACCUACACCUGACGACU |
| | | | ACGACGAGGAAGAUGACGCUGGCGUCAGCGAGAGA |
| | | | ACCCCUGUUUCUGUGCCUCCUCCUACGCCUCCUCG |
| | | | UAGACCUCCUGUUGCUCCUCCAACACACCCCAGAG |
| | | | UGAUCCCUGAAGUGUCUCACGUGCGGGGCGUGACC |
| | | | GUGCACAUGGAAACACCUGAGGCCAUCCUGUUCGC |
| | | | CCCUGGCGAGACAUUUGGCACCAACGUGUCCAUCC |
| | | | ACGCUAUCGCCCACGACGAUGGCCCUUACGCCAUG |
| | | | GAUGUCGUGUGGAUGAGAUUCGACGUGCCCAGCAG |
| | | | CUGCGCCGAGAUGAGAAUCUACGAGGCCUGCCUGU |
| | | | AUCACCCUCAGCUGCCCGAAUGUCUGAGCCCUGCU |
| | | | GAUGCCCCUUGUGCCGUUAGCAGCUGGGCCUAUAG |
| | | | ACUGGCCGUGCGGUCUUAUGCCGGCUGCUCUAGAA |
| | | | CAACCCCUCCUCCUCGGUGUUUCGCCGAGGCCAGA |
| | | | AUGGAACCUGUUCCUGGACUGGCCUGGCUGGCCUC |
| | | | CACAGUGAACCUGGAAUUUCAGCACGCCUCUCCAC |
| | | | AGCACGCCGGCCUGUAUCUGUGUGUGGUGUACGUG |
| | | | GACGAUCACAUCCACGCCUGGGGCCACAUGACCAU |
| | | | CUCUACAGCCGCUCAGUACCGGAACGCCGUGGUUG |
| | | | AACAGCAUCUGCCCCAGAGACAGCCCGAGCCUGUG |
| | | | GAACCUACAAGACCUCAUGUUCGGGCCUGAUAA |
| 26 | HSV-2 gE | Version 2 | CGCACCUCUUGGAAACGCGUUACUUCCGGGGAGGA |
| | [US8] | | CGUUGUCCUCCUUCCAGCACCCGCAGGACCUGAGG |
| | | | AAAGGACUAGGGCCCACAAGCUGCUGUGGGCCGCU |
| | | | GAACCUCUGGAUGCCUGUGGUCCUCUGAGACCUAG |
| | | | CUGGGUCGCCCUUUGGCCACCUAGACGCGUUCUGG |
| | | | AGACGGUCGUGGAUGCCGCGUGCAUGCGUGCACCC |
| | | | GAACCUCUGGCCAUCGCCUAUAGUCCCCCUUUUCC |
| | | | CGCUGGCGACGAGGGGCUUUACUCCGAACUGGCCU |
| | | | GGCGGGAUAGGGUGGCGGUGGUGAACGAGAGCCUC |
| | | | GUCAUCUACGGUGCUCUGGAAACCGACUCAGGACU |
| | | | GUAUACGCUCAGCGUUGUUGGCCUCUCCGAUGAGG |
| | | | CUCGACAGGUUGCCUCCGUAGUGCUGGUCGUAGAA |
| | | | CCAGCCCCCGUACCAACACCCACACCCGACGACUAC |
| | | | GACGAGGAGGACGACGCUGGAGUUAGCGAAAGAAC |
| | | | ACCGGUGAGUGUGCCACCUCCCACACCGCCAAGGA |
| | | | GACCCCCAGUAGCACCUCCAACCCAUCCGAGAGUG |
| | | | AUUCCCGAGGUCAGCCAUGUGCGCGGCGUAACUGU |
| | | | GCACAUGGAGACGCCCGAAGCGAUACUGUUUGCCC |
| | | | CUGGAGAGACAUUCGGCACCAAUGUGUCCAUACAC |
| | | | GCAAUUGCGCACGAUGAUGGCCCAUACGCUAUGGA |
| | | | CGUCGUCUGGAUGAGGUUCGAUGUGCCUUCUUCUU |
| | | | GCGCCGAGAUGAGGAUCUACGAGGCAUGCCUGUAU |
| | | | CACCCCCAAUUGCCGGAGUGUCUGUCUCCCGCAGA |
| | | | UGCACCGUGUGCAGUGAGUAGCUGGGCUUAUCGGU |
| | | | UGGCUGUCCGGAGUUAUGCUGGGUGUUCACGGACC |
| | | | ACCCCACCUCCACGUUGCUUUGCUGAAGCCAGAAU |
| | | | GGAACCCGUGCCUGGUCUGGCUUGGCUGGCAUCAA |
| | | | CUGUCAACCUGGAGUUCCAGCAUGCCUCUCCACAG |
| | | | CACGCAGGCCUGUAUCUCUGCGUGGUGUACGUUGA |
| | | | CGAUCACAUCCAUGCGUGGGGGCAUAUGACCAUCA |
| | | | GCACAGCUGCCCAGUACCGCAAUGCCGUCGUGGAG |
| | | | CAGCACCUCCCCCAACGGCAGCCAGAACCAGUGGA |
| | | | GCCCACUCGGCCUCAUGUGCGAGCCUGA |
| 149 | HSV-2 gE | Version | CGCACCUCUUGGAAACGCGUUACUUCCGGGGAGGA |
| | [US8] | 2.1 | CGUUGUCCUCCUUCCAGCACCCGCAGGACCUGAAG |
| | | | AGAGGACUAGGGCCCACAAGCUGCUGUGGGCCGCU |
| | | | GAACCUCUGGAUGCCUGUGGUCCUCUGAGACCUAG |
| | | | CUGGGUCGCCCUUUGGCCACCUAGACGCGUUCUGG |
| | | | AGACGGUCGUGGAUGCCGCGUGCAUGCGUGCACCC |
| | | | GAACCUCUGGCCAUCGCCUAUAGUCCCCCUUUUCC |
| | | | CGCUGGCGACGAGGGGCUUUACUCCGAACUGGCCU |
| | | | GGCGGGAUAGGGUGGCGGUGGUGAACGAGAGCCUC |
| | | | GUCAUCUACGGUGCUCUGGAAACCGACUCAGGACU |
| | | | GUAUACGCUCAGCGUUGUUGGCCUCUCCGAUGAGG |
| | | | CUCGACAGGUUGCCUCCGUAGUGCUGGUCGUAGAA |
| | | | CCAGCCCCCGUACCAACACCCACACCCGACGACUAC |
| | | | GACGAAGAGGACGACGCUGGAGUUAGCGAAAGAAC |
| | | | ACCGGUGAGUGUGCCACCUCCCACACCGCCAAGGA |
| | | | GACCCCCAGUAGCACCUCCAACCCAUCCGAGAGUG |
| | | | AUUCCCGAGGUCAGCCAUGUGCGCGGCGUAACUGU |
| | | | GCACAUGGAGACGCCCGAAGCGAUACUGUUUGCCC |
| | | | CUGGAGAGACAUUCGGCACCAAUGUGUCCAUACAC |
| | | | GCAAUUGCGCACGAUGAUGGCCCAUACGCUAUGGA |
| | | | CGUCGUCUGGAUGAGGUUCGAUGUGCCUUCUUCUU |
| | | | GCGCCGAGAUGAGGAUCUACGAGGCAUGCCUGUAU |
| | | | CACCCCCAAUUGCCGGAGUGUCUGUCUCCCGCAGA |
| | | | UGCACCGUGUGCAGUGAGUAGCUGGGCUUAUCGGU |
| | | | UGGCUGUCCGGAGUUAUGCUGGGUGUUCACGGACC |
| | | | ACCCCACCUCCACGUUGCUUUGCUGAAGCCAGAAU |
| | | | GGAACCCGUGCCUGGUCUGGCUUGGCUGGCAUCAA |
| | | | CUGUCAACCUGGAGUUCCAGCAUGCCUCUCCACAG |
| | | | CACGCAGGCCUGUAUCUCUGCGUGGUGUACGUUGA |
| | | | CGAUCACAUCCAUGCGUGGGGGCAUAUGACCAUCA |
| | | | GCACAGCUGCCCAGUACCGCAAUGCCGUCGUGGAG |
| | | | CAGCACCUCCCCCAACGGCAGCCAGAACCAGUGGA |
| | | | GCCCACUCGGCCUCAUGUGCGAGCCUGA |
| 341 | HSV-2 gE | Version | CGCACCUCUUGGAAACGCGUUACUUCCGGGGAGGA |
| | [US8] | 2.2 | CGUUGUCCUCCUUCCAGCACCCGCAGGACCUGAGG |
| | | | AAAGGACUAGGGCCCACAAGCUGCUGUGGGCCGCU |
| | | | GAACCUCUGGAUGCCUGUGGUCCUCUGAGACCUAG |
| | | | CUGGGUCGCCCUUUGGCCACCUAGACGCGUUCUGG |
| | | | AGACGGUCGUGGAUGCCGCGUGCAUGCGUGCACCC |
| | | | GAACCUCUGGCCAUCGCCUAUAGUCCCCCUUUUCC |
| | | | CGCUGGCGACGAGGGGCUUUACUCCGAACUGGCCU |
| | | | GGCGGGAUAGGGUGGCGGUGGUGAACGAGAGCCUC |
| | | | GUCAUCUACGGUGCUCUGGAAACCGACUCAGGACU |
| | | | GUAUACGCUCAGCGUUGUUGGCCUCUCCGAUGAGG |
| | | | CUCGACAGGUUGCCUCCGUAGUGCUGGUCGUAGAA |
| | | | CCAGCCCCCGUACCAACACCCACACCCGACGACUAC |
| | | | GACGAGGAGGACGACGCUGGAGUUAGCGAAAGAAC |
| | | | ACCGGUGAGUGUGCCACCUCCCACACCGCCAAGGA |
| | | | GACCCCCAGUAGCACCUCCAACCCAUCCGAGAGUG |
| | | | AUUCCCGAGGUCAGCCAUGUGCGCGGCGUAACUGU |
| | | | GCACAUGGAGACGCCCGAAGCGAUACUGUUUGCCC |
| | | | CUGGAGAGACAUUCGGCACCAAUGUGUCCAUACAC |
| | | | GCAAUUGCGCACGAUGAUGGCCCAUACGCUAUGGA |
| | | | CGUCGUCUGGAUGAGGUUCGAUGUGCCUUCUUCUU |
| | | | GCGCCGAGAUGAGGAUCUACGAGGCAUGCCUGUAU |
| | | | CACCCCCAAUUGCCGGAGUGUCUGUCUCCCGCAGA |
| | | | UGCACCGUGUGCAGUGAGUAGCUGGGCUUAUCGGU |
| | | | UGGCUGUCCGGAGUUAUGCUGGGUGUUCACGGACC |
| | | | ACCCCACCUCCACGUUGCUUUGCUGAAGCCAGAAU |
| | | | GGAACCCGUGCCUGGUCUGGCUUGGCUGGCAUCAA |
| | | | CUGUCAACCUGGAGUUCCAGCAUGCCUCUCCACAG |
| | | | CACGCAGGCCUGUAUCUCUGCGUGGUGUACGUUGA |
| | | | CGAUCACAUCCAUGCGUGGGGGCAUAUGACCAUCA |
| | | | GCACAGCUGCCCAGUACCGCAAUGCCGUCGUGGAG |
| | | | CAGCACCUCCCCCAACGGCAGCCAGAACCAGUGGA |
| | | | GCCCACUCGGCCUCAUGUGCGAGCCUGAUAA |
| 283 | HSV-2 gE | Version | CGCACCUCUUGGAAACGCGUUACUUCCGGGGAGGA |
| | [US8] | 2.2 | CGUUGUCCUCCUUCCAGCACCCGCAGGACCUGAGG |
| | | | AAAGGACUAGGGCCCACAAGCUGCUGUGGGCCGCU |
| | | | GAACCUCUGGAUGCCUGUGGUCCUCUGAGACCUAG |
| | | | CUGGGUCGCCCUUUGGCCACCUAGACGCGUUCUGG |
| | | | AGACGGUCGUGGAUGCCGCGUGCAUGCGUGCACCC |
| | | | GAACCUCUGGCCAUCGCCUAUAGUCCCCCUUUUCC |
| | | | CGCUGGCGACGAGGGGCUUUACUCCGAACUGGCCU |
| | | | GGCGGGAUAGGGUGGCGGUGGUGAACGAGAGCCUC |
| | | | GUCAUCUACGGUGCUCUGGAAACCGACUCAGGACU |
| | | | GUAUACGCUCAGCGUUGUUGGCCUCUCCGAUGAGG |
| | | | CUCGACAGGUUGCCUCCGUAGUGCUGGUCGUAGAA |
| | | | CCAGCCCCCGUACCAACACCCACACCCGACGACUAC |
| | | | GACGAGGAGGACGACGCUGGAGUUAGCGAAAGAAC |
| | | | ACCGGUGAGUGUGCCACCUCCCACACCGCCAAGGA |
| | | | GACCCCCAGUAGCACCUCCAACCCAUCCGAGAGUG |
| | | | AUUCCCGAGGUCAGCCAUGUGCGCGGCGUAACUGU |
| | | | GCACAUGGAGACGCCCGAAGCGAUACUGUUUGCCC |
| | | | CUGGAGAGACAUUCGGCACCAAUGUGUCCAUACAC |
| | | | GCAAUUGCGCACGAUGAUGGCCCAUACGCUAUGGA |
| | | | CGUCGUCUGGAUGAGGUUCGAUGUGCCUUCUUCUU |
| | | | GCGCCGAGAUGAGGAUCUACGAGGCAUGCCUGUAU |
| | | | CACCCCCAAUUGCCGGAGUGUCUGUCUCCCGCAGA |
| | | | UGCACCGUGUGCAGUGAGUAGCUGGGCUUAUCGGU |
| | | | UGGCUGUCCGGAGUUAUGCUGGGUGUUCACGGACC |
| | | | ACCCCACCUCCACGUUGCUUUGCUGAAGCCAGAAU |
| | | | GGAACCCGUGCCUGGUCUGGCUUGGCUGGCAUCAA |
| | | | CUGUCAACCUGGAGUUCCAGCAUGCCUCUCCACAG |
| | | | CACGCAGGCCUGUAUCUCUGCGUGGUGUACGUUGA |
| | | | CGAUCACAUCCAUGCGUGGGGGCAUAUGACCAUCA |
| | | | GCACAGCUGCCCAGUACCGCAAUGCCGUCGUGGAG |
| | | | CAGCACCUCCCCCAACGGCAGCCAGAACCAGUGGA |
| | | | GCCCACUCGGCCUCAUGUGCGAGCCUGAUAA |
| 27 | HSV-2 gE | Version 3 | AGAACCUCCUGGAAAAGAGUGACCUCCGGCGAAGA |
| | [US8] | | UGUGGUGCUGCUGCCCGCCCCCGCCGGCCCCGAAG |
| | | | AAAGAACCAGAGCCCACAAACUGCUGUGGGCCGCC |
| | | | GAACCCCUGGAUGCCUGCGGCCCCCUCAGACCCUC |
| | | | CUGGGUGGCCCUGUGGCCCCCUAGAAGGGUGCUGG |
| | | | AAACCGUGGUGGAUGCCGCCUGCAUGAGAGCCCCC |
| | | | GAACCCCUGGCCAUCGCCUACUCCCCUCCCUUCCCC |
| | | | GCCGGCGAUGAAGGCCUGUACUCCGAACUGGCCUG |
| | | | GAGAGAUAGAGUGGCCGUGGUGAACGAAUCCCUGG |
| | | | UGAUCUACGGCGCCCUGGAAACCGAUUCCGGCCUG |
| | | | UACACCCUGUCCGUGGUGGGCCUGUCCGAUGAAGC |
| | | | CAGACAGGUGGCCUCCGUGGUGCUGGUGGUGGAAC |
| | | | CCGCCCCCGUGCCCACCCCCACCCCCGAUGAUUACG |
| | | | AUGAAGAAGAUGAUGCCGGCGUGUCCGAAAGAACC |
| | | | CCCGUGUCCGUGCCCCCUCCCACCCCUCCCCGCAGA |
| | | | CCCCCUGUGGCCCCUCCCACCCACCCCAGAGUGAUC |
| | | | CCCGAAGUGUCCCACGUGAGAGGCGUGACCGUGCA |
| | | | CAUGGAAACCCCCGAAGCCAUCCUGUUCGCCCCCG |
| | | | GCGAAACCUUCGGCACCAACGUGUCCAUCCACGCC |
| | | | AUCGCCCACGAUGAUGGCCCCUACGCCAUGGAUGU |
| | | | GGUGUGGAUGAGAUUCGAUGUGCCCUCCUCCUGCG |
| | | | CCGAAAUGAGAAUCUACGAAGCCUGCCUGUACCAC |
| | | | CCCCAGCUGCCCGAAUGCCUGUCCCCCGCCGAUGCC |
| | | | CCCUGCGCCGUGUCCUCCUGGGCCUACAGACUGGC |
| | | | CGUGAGAUCCUACGCCGGCUGCUCCAGAACCACCC |
| | | | CUCCCCCUAGAUGCUUCGCCGAAGCCAGAAUGGAA |
| | | | CCCGUGCCCGGCCUGGCCUGGCUGGCCUCCACCGU |
| | | | GAACCUGGAAUUCCAGCACGCCAGCCCCCAGCACG |
| | | | CCGGCCUGUACCUGUGCGUGGUGUACGUGGAUGAU |
| | | | CACAUCCACGCCUGGGGCCACAUGACCAUCUCCAC |
| | | | CGCCGCCCAGUACAGAAACGCCGUGGUGGAACAGC |
| | | | ACCUGCCCCAGAGACAGCCCGAACCCGUGGAACCC |
| | | | ACCAGACCCCACGUGAGAGCCUGA |
| 284 | HSV-2 gE | Version | AGAACCUCUUGGAAAAGAGUGACCUCUGGAGAAGA |
| | [US8] | 3.1 | UGUGGUGCUGCUGCCAGCUCCAGCUGGACCAGAAG |
| | | | AAAGAACCAGAGCACACAAACUGCUGUGGGCUGCU |
| | | | GAACCUCUGGAUGCUUGUGGACCUCUGAGACCUUC |
| | | | UUGGGUGGCUCUGUGGCCACCAAGAAGGGUGCUGG |
| | | | AAACAGUGGUGGAUGCUGCUUGCAUGAGAGCACCU |
| | | | GAACCUCUGGCAAUUGCAUACUCUCCUCCUUUUCC |
| | | | AGCUGGAGAUGAAGGACUGUAUUCUGAACUGGCU |
| | | | UGGAGAGACAGAGUGGCUGUGGUGAAUGAAUCUC |
| | | | UGGUGAUCUAUGGAGCACUGGAAACAGAUUCUGG |
| | | | ACUGUACACCCUGUCUGUGGUGGGACUGUCUGAUG |
| | | | AAGCAAGACAGGUGGCAUCUGUGGUGCUGGUGGU |
| | | | GGAACCAGCUCCAGUGCCAACCCCAACCCCAGAUG |
| | | | AUUAUGAUGAAGAAGAUGAUGCUGGAGUGUCUGA |
| | | | AAGAACCCCAGUGUCUGUGCCACCACCAACCCCAC |
| | | | CAAGAAGACCACCAGUGGCUCCACCAACCCACCCA |
| | | | AGAGUGAUCCCAGAAGUGUCUCAUGUGAGAGGAG |
| | | | UGACAGUGCACAUGGAAACCCCUGAAGCAAUCCUG |
| | | | UUUGCACCUGGAGAAACCUUUGGAACCAAUGUGUC |
| | | | CAUCCAUGCAAUUGCACAUGAUGAUGGACCUUAUG |
| | | | CAAUGGAUGUGGUGUGGAUGAGAUUUGAUGUGCC |
| | | | UUCUUCUUGUGCUGAAAUGAGAAUCUAUGAAGCU |
| | | | UGCCUGUACCACCCUCAGCUGCCUGAAUGCCUGUC |
| | | | UCCUGCUGAUGCUCCUUGUGCUGUGUCUUCUUGGG |
| | | | CUUACAGACUGGCUGUGAGAUCUUAUGCUGGAUGC |
| | | | UCCAGAACCACCCCACCACCAAGAUGCUUUGCUGA |
| | | | AGCAAGAAUGGAACCUGUGCCUGGACUGGCAUGGC |
| | | | UGGCAUCCACAGUGAAUCUGGAAUUUCAGCAUGCU |
| | | | UCUCCUCAGCAUGCUGGACUGUACCUGUGUGUGGU |
| | | | GUAUGUGGACGAUCACAUCCAUGCUUGGGGACACA |
| | | | UGACCAUCAGCACAGCUGCUCAGUACAGAAAUGCU |
| | | | GUGGUGGAACAGCACCUGCCACAGAGACAGCCAGA |
| | | | ACCAGUGGAACCAACCAGACCACAUGUGAGAGCUU |
| | | | GAUAA |
| 285 | HSV-2 gE | Version 4 | CGGACCAGCUGGAAAAGGGUGACAUCUGGGGAAGA |
| | [US8] | | UGUCGUGCUCCUUCCUGCGCCUGCUGGCCCAGAAG |
| | | | AACGCACUAGGGCCCACAAGCUUCUGUGGGCCGCG |
| | | | GAGCCACUGGAUGCGUGUGGUCCCCUGCGACCCUC |
| | | | UUGGGUGGCAUUGUGGCCACCCCGACGCGUACUCG |
| | | | AAACGGUCGUUGACGCCGCCUGUAUGAGAGCGCCA |
| | | | GAGCCCCUCGCCAUUGCCUACAGCCCGCCUUUUCC |
| | | | CGCCGGUGACGAAGGACUGUACAGUGAGCUGGCCU |
| | | | GGCGCGAUAGGGUCGCCGUAGUUAACGAGUCCCUG |
| | | | GUGAUAUACGGCGCUCUGGAAACCGACAGUGGCUU |
| | | | GUACACCCUGUCCGUUGUAGGCCUGUCCGAUGAAG |
| | | | CACGGCAAGUGGCUUCCGUGGUACUGGUCGUAGAG |
| | | | CCCGCACCAGUUCCCACACCCACCCCGGAUGACUAC |
| | | | GAUGAGGAGGACGAUGCCGGUGUGAGUGAGCGUA |
| | | | CACCUGUUAGCGUACCUCCACCAACUCCUCCACGC |
| | | | CGCCCUCCUGUUGCACCGCCUACACAUCCCCGUGU |
| | | | GAUUCCUGAAGUGUCACACGUUAGAGGGGUGACAG |
| | | | UCCACAUGGAGACUCCCGAAGCCAUCCUCUUUGCA |
| | | | CCAGGCGAGACUUUUGGGACCAAUGUGAGCAUUCA |
| | | | CGCCAUAGCUCACGAUGACGGGCCCUAUGCCAUGG |
| | | | ACGUGGUGUGGAUGAGGUUCGAUGUGCCCUCAUCA |
| | | | UGCGCUGAGAUGCGGAUCUACGAAGCUUGCCUGUA |
| | | | UCACCCACAGCUUCCCGAGUGCUUGUCUCCCGCUG |
| | | | ACGCCCCUUGUGCUGUUAGCUCUUGGGCUUAUCGG |
| | | | CUUGCCGUCAGGAGCUAUGCUGGAUGCUCCAGAAC |
| | | | CACACCUCCACCGAGGUGUUUCGCCGAGGCCAGAA |
| | | | UGGAGCCUGUGCCAGGACUGGCCUGGCUGGCAAGU |
| | | | ACUGUGAACCUGGAAUUCCAGCACGCAUCACCUCA |
| | | | GCAUGCAGGCCUGUACCUCUGCGUUGUCUAUGUCG |
| | | | ACGACCAUAUCCACGCAUGGGGCCAUAUGACCAUC |
| | | | AGCACGGCAGCACAGUAUCGGAAUGCUGUGGUCGA |
| | | | GCAGCACUUGCCCCAGCGACAACCCGAACCAGUGG |
| | | | AGCCAACCAGACCGCAUGUGCGGGCCUGA |

Provided herein is a polyribonucleotide encoding a polypeptide. In some embodiments, a polypeptide comprises one or more HSV glycoprotein C (gC) antigens or antigenic fragments thereof. In some embodiments, a polypeptide comprises an antigenic portion of HSV gC. In some embodiments, an antigenic portion of HSV gC comprises an amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 1 or a portion thereof. In some embodiments, an antigenic portion of HSV gC has an amino acid sequence that is identical to the amino acid sequence of SEQ ID NO: 1. In some embodiments, an antigenic portion of HSV gC comprises an amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 260. In some embodiments, an antigenic portion of HSV gC has an amino acid sequence that is identical to the amino acid sequence of SEQ ID NO: 260.

In some embodiments, a polyribonucleotide comprises a ribonucleic acid sequence that is at least 85%, at least 90%, at least 91%, at least 92%, at least 93%, at least 94%, at least 95%, at least 96%, at least 97%, at least 98%, at least 99%, or 100% identical to the ribonucleic acid sequence of any one of SEQ ID NO: 16-19, 147 and 274-281.

In some embodiments, a polypeptide comprises one or more HSV glycoprotein D (gD) antigens or antigenic fragments thereof. In some embodiments, a polypeptide comprises an antigenic portion of HSV gD. In some embodiments, an antigenic portion of HSV gD comprises an amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 2 or a portion thereof. In some embodiments, an antigenic portion of HSV gD has an amino acid sequence that is identical to the amino acid sequence of SEQ ID NO: 2.

In some embodiments, a polyribonucleotide comprises a ribonucleic acid sequence that is at least 85%, at least 90%, at least 91%, at least 92%, at least 93%, at least 94%, at least 95%, at least 96%, at least 97%, at least 98%, at least 99%, or 100% identical to the ribonucleic acid sequence of any one of SEQ ID NO: 20-23, 143, and 286.

In some embodiments, a polypeptide comprises one or more HSV glycoprotein E (gE) antigens or antigenic fragments thereof. In some embodiments, a polypeptide comprises an antigenic portion of HSV gE. In some embodiments, an antigenic portion of HSV gE comprises an amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 3 or a portion thereof. In some embodiments, an antigenic portion of HSV gE has an amino acid sequence that is identical to the amino acid sequence of SEQ ID NO: 3.

In some embodiments, a polyribonucleotide comprises a ribonucleic acid sequence that is at least 85%, at least 90%, at least 91%, at least 92%, at least 93%, at least 94%, at least 95%, at least 96%, at least 97%, at least 98%, at least 99%, or 100% identical to the ribonucleic acid sequence of any one of SEQ ID NO: 24-27 and 282-285.

### B. Secretory Signals

Provided herein are polypeptides comprising (i) an HSV antigen or an antigenic fragment thereof and (ii) a secretory signal. Also provided herein are polyribonucleotides that encoding a polypeptide comprising (i) an HSV antigen or an antigenic fragment thereof and (ii) a secretory signal. In some embodiments, a secretory signal is functional in mammalian cells. In some embodiments, a secretory signal comprises or consists of a human secretory signal. In some embodiments, a secretory signal comprises or consists of an IL2 secretory signal.

In some embodiments, a secretory signal comprises or consists of a viral secretory signal. In some embodiments, a viral secretory signal comprises or consists of an HSV secretory signal (e.g., an HSV-1 or HSV-2 secretory signal). In some embodiments, a secretory signal comprises or consists of an HSV-1 secretory signal. In some embodiments, a secretory signal comprises or consists of an HSV-2 secretory signal.

In some embodiments, an HSV secretory signal comprises or consists of an HSV glycoprotein D (gD) secretory signal (e.g., an HSV-1 or HSV-2 gD secretory signal). In some embodiments, an HSV secretory signal comprises or consists of an HSV-1 gD secretory signal. In some embodiments, an HSV-1 gD secretory signal comprises one or more additional amino acids. In some embodiments, an HSV-1 gD secretory signal comprises KY at the C terminus of the signal sequence. In some embodiments, an HSV secretory signal comprises or consists of an HSV-2 gD secretory signal. In some embodiments, an HSV-2 gD secretory signal comprises one or more additional amino acids. In some embodiments, an HSV-2 gD secretory signal comprises KYA or KYALA at the C terminus of the signal sequence.

In some embodiments, an HSV secretory signal comprises or consists of an HSV glycoprotein C (gC) secretory signal (e.g., an HSV-1 or HSV-2 gC secretory signal). In some embodiments, an HSV secretory signal comprises or consists of an HSV-2 gC secretory signal.

In some embodiments, an HSV secretory signal comprises or consists of an HSV glycoprotein E (gE) secretory signal (e.g., an HSV-1 or HSV-2 gE secretory signal). In some embodiments, an HSV secretory signal comprises or consists of an HSV-1 gE secretory signal. In some embodiments, an HSV secretory signal comprises or consists of an HSV-2 gE secretory signal. In some embodiments, an HSV-2 gE secretory signal comprises one or more additional amino acids. In some embodiments, an HSV-2 gE secretory signal comprises RTS. In some embodiments, an HSV-2 secretory signal comprises A20V, A21V, A22V substitutions.

In some embodiments, an HSV secretory signal comprises or consists of an HSV glycoprotein B (gB) secretory signal (e.g., an HSV-1 or HSV-2 gB secretory signal). In some embodiments, an HSV secretory signal comprises or consists of an HSV-1 gB secretory signal. In some embodiments, an HSV-1 gB secretory signal comprises one or more additional amino acids. In some embodiments, an HSV-1 gB secretory signal comprises AP at the C terminus of the signal sequence. In some embodiments, an HSV secretory signal comprises or consists of an HSV-2 gB secretory signal.

In some embodiments, an HSV secretory signal comprises or consists of an HSV glycoprotein I (gI) secretory signal (e.g., an HSV-1 or HSV-2 gI secretory signal). In some embodiments, an HSV secretory signal comprises or consists of an HSV-1 gI secretory signal. In some embodiments, an HSV-1 gI secretory signal comprises one or more additional amino acids. In some embodiments, an HSV secretory signal comprises or consists of an HSV-2 gI secretory signal. In some embodiments, an HSV-2 gI secretory signal comprises an additional leucine residue at the C terminus of the signal sequence.

In some embodiments, a secretory signal comprises or consists of an Ebola spike glycoprotein (EboZ). In some embodiments, an EboZ secretory signal comprises one or more additional amino acids. In some embodiments, an Eboz secretory signal comprises IP at the C terminus of the signal sequence.

In some embodiments, a secretory signal is characterized by a length of about 15 to 30 amino acids.

In some embodiments, a secretory signal is positioned at the N-terminus of a polyribonucleotide. In some embodiments, a secretory signal preferably allows transport of a polyribonucleotide with which it is associated into a defined cellular compartment, preferably a cell surface, endoplasmic reticulum (ER) or endosomal-lysosomal compartment.

In some embodiments, polyribonucleotides comprising an HSV antigen do not comprise a secretory signal. In some embodiments, polyribonucleotides comprising an HSV antigen further comprise a codon initiation start site.

In some embodiments, a secretory signal is one listed in Table 7, or a secretory signal having 1, 2, 3, 4, or 5 amino acid differences relative thereto. In some embodiments, a secretory signal is selected from those included in the Table 7 below and/or those encoded by the sequences in Table 8 and/or Table 9 below.

TABLE 7
Example secretory signals

| SEQ | | |
|---|---|---|
| ID NO: | Secretory signal | Sequence (Amino Acid) |
| 28 | IL2 | MRMQLLLLIALSLALVTNS |
| 72 | MHC Class II | MAISGVPVLGFFIIAVLMSAQESWA |
| 29 | HSV-2 gD | MGRLTSGVGTAALLVVAVGLRVVCA |
| 354 | HSV-2 gD + KY | MGRLTSGVGTAALLVVAVGLRVVCAKY |
| 30 | HSV-2 gD + KYA | MGRLTSGVGTAALLVVAVGLRVVCAKYA |
| 355 | HSV-2 gD + KYAL | MGRLTSGVGTAALLVVAVGLRVVCAKYAL |
| 31 | HSV-2 gD + KYALA | MGRLTSGVGTAALLVVAVGLRVVCAKYALA |
| 356 | HSV-1 gD | MGGAAARLGAVILFVVIVGLHGVRG |
| 213 | HSV-1 gD + KY | MGGAAARLGAVILFVVIVGLHGVRGKY |
| 32 | HSV-2 gC | MALGRVGLAVGLWGLLWVGVVVVLANA |
| 357 | HSV-1 gC | MDRGAVVGFLLGVCVVSCLA |
| 358 | HSV-1 gE | MDRGAVVGFLLGVCVVSCLA |
| 33 | HSV-2 gE | MARGAGLVFFVGVWVVSCLAAAP |
| 292 | HSV-2 gE − LVVVP | MARGAGLVFFVGVWVVSCLVVVP |
| 216 | HSV-2 gE + RTS | MARGAGLVFFVGVWVVSCLAAAPRTS |
| 359 | HSV-1 gB | MHQGAPSWGRRWFVVWALLGLTLGVLVASA |
| 214 | HSV-1 gB + AP | MHQGAPSWGRRWFVVWALLGLTLGVLVASAAP |
| 360 | HSV-2 gB | MRGGGLICALVVGALVAAVASA |
| 361 | HSV-2 gI (1-18) | MPGRSLQGLAILGLWVCA |
| 215 | HSV-2 gI | MPGRSLQGLAILGLWVCATG |
| 362 | HSV-2 gI + L | MPGRSLQGLAILGLWVCATGL |
| 217 | Ebola spike | MGVTGILQLPRDRFKRTSFFLWVIILFQRTFSIP |
| | glycoprotein | |
| | (EboZ) + IP | |

TABLE 8
Example Deoxyribonucleic Acid Sequences
encoding secretory signals

| SEQ | | | |
|---|---|---|---|
| ID | Secretory | | |
| NO: | signal | Versions | Sequence (Nucleotide) |
| 34 | IL2 | Wild type | ATGCGCATGCAGCTGCTGCTGCTGATCGCCC |
| | | | TGTCCCTGGCCCTGGTGACCAACTCC |
| 35 | IL2 | Version 1 | ATGAGAATGCAGCTGCTGCTCCTGATCGCCC |
| | | | TGTCTCTGGCCCTGGTCACCAAT |
| 36 | IL2 | Version 2 | ATGCGCATGCAACTGCTCCTGCTGATTGCGT |
| | | | TGAGCCTTGCCCTGGTGACCAACAGC |
| 329 | IL2 | Version | ATGCGAATGCAGCTTCTGCTGCTCATTGCCT |
| | | 2.1 | TGTCCCTTGCCTTGGTGACCAACTCA |
| 37 | IL2 | Version 3 | ATGAGAATGCAGCTGCTGCTGCTGATCGCCC |
| | | | TGTCCCTGGCCCTGGTGACCAACTCC |
| 38 | HSV-2 gD | Wild type | ATGGGCCGCCTGACCTCCGGCGTGGGCACC |
| | | | GCCGCCCTGCTGGTGGTGGCCGTGGGCCTGC |
| | | | GCGTGGTGTGCGCC |
| 39 | HSV-2 gD | Version 1 | ATGGGCAGACTGACATCTGGCGTGGGAACA |
| | | | GCTGCTCTGCTGGTGGTTGCTGTGGGCCTGA |
| | | | GAGTCGTGTGTGCC |
| 40 | HSV-2 gD | Version 2 | ATGGGGAGACTCACATCAGGCGTAGGAACC |
| | | | GCTGCCCTGTTGGTCGTGGCCGTTGGTCTGA |
| | | | GAGTTGTGTGTGCC |
| 41 | HSV-2 gD | Version 3 | ATGGGCAGACTGACCTCCGGCGTGGGCACC |
| | | | GCCGCCCTGCTGGTGGTGGCCGTGGGCCTGA |
| | | | GAGTGGTGTGCGCC |
| 42 | HSV-2 gD − KYA | Wild type | ATGGGCCGCCTGACCTCCGGCGTGGGCACC |
| | | | GCCGCCCTGCTGGTGGTGGCCGTGGGCCTGC |
| | | | GCGTGGTGTGCGCCAAGTACGCC |
| 43 | HSV-2 gD − KYA | Version 1 | ATGGGCAGACTGACATCTGGCGTGGGAACA |
| | | | GCTGCTCTGCTGGTGGTTGCTGTGGGCCTGA |
| | | | GAGTCGTGTGTGCCAAATACGCC |
| 44 | HSV-2 gD − KYA | Version 2 | ATGGGGAGACTCACATCAGGCGTAGGAACC |
| | | | GCTGCCCTGTTGGTCGTGGCCGTTGGTCTGA |
| | | | GAGTTGTGTGTGCCAAATATGCT |
| 45 | HSV-2 gD − KYA | Version 3 | ATGGGCAGACTGACCTCCGGCGTGGGCACC |
| | | | GCCGCCCTGCTGGTGGTGGCCGTGGGCCTGA |
| | | | GAGTGGTGTGCGCCAAATACGCC |
| 331 | HSV-2 gD − KYAL | Version 2 | ATGGGGAGACTCACATCAGGCGTAGGAACC |
| | | | GCTGCCCTGTTGGTCGTGGCCGTTGGTCTGA |
| | | | GAGTTGTGTGTGCCAAATATGCTCTG |
| 332 | HSV-2 gD − KYAL | Version 3 | ATGGGCAGACTGACCTCCGGCGTGGGCACC |
| | | | GCCGCCCTGCTGGTGGTGGCCGTGGGCCTGA |
| | | | GAGTGGTGTGCGCCAAATACGCCCTG |
| 46 | HSV-2 gD − KYALA | Wild type | ATGGGCCGCCTGACCTCCGGCGTGGGCACC |
| | | | GCCGCCCTGCTGGTGGTGGCCGTGGGCCTGC |
| | | | GCGTGGTGTGCGCCAAGTACGCCCTGGCC |
| 47 | HSV-2 gD − KYALA | Version 1 | ATGGGCAGACTGACATCTGGCGTGGGAACA |
| | | | GCTGCTCTGCTGGTGGTTGCTGTGGGCCTGA |
| | | | GAGTCGTGTGTGCCAAATACGCCCTGGCC |
| 48 | HSV-2 gD − KYALA | Version 2 | ATGGGGAGACTCACATCAGGCGTAGGAACC |
| | | | GCTGCCCTGTTGGTCGTGGCCGTTGGTCTGA |
| | | | GAGTTGTGTGTGCCAAATATGCTCTG |
| 293 | HSV-2 gD − KYALA | Version | ATGGGGAGACTCACATCCGGAGTGGGCACT |
| | | 2.1 | GCTGCCCTGTTGGTCGTGGCCGTTGGTCTGA |
| | | | GAGTTGTGTGTGCCAAATATGCTCTGGCA |
| 49 | HSV-2 gD − KYALA | Version 3 | ATGGGCAGACTGACCTCCGGCGTGGGCACC |
| | | | GCCGCCCTGCTGGTGGTGGCCGTGGGCCTGA |
| | | | GAGTGGTGTGCGCCAAATACGCCCTG |
| 138 | HSV-2 gC | Version 2 | ATGGCCTTGGGGAGAGTGGGCCTTGCAGTG |
| | | | GGTCTGTGGGGACTGCTCTGGGTTGGCGTAG |
| | | | TCGTCGTGCTGGCTAACGCA |
| 294 | HSV-2 gC | Version | ATGGCACTAGGGAGAGTGGGATTAGCTGTG |
| | | 2.1 | GGTCTGTGGGGACTGCTCTGGGTAGGAGTCG |
| | | | TCGTCGTGCTGGCTAACGCA |
| 295 | HSV-2 gC | Version | ATGGCCTTGGGGAGAGTCGGCCTTGCAGTG |
| | | 2.2 | GGACTGTGGGGTCTGCTGTGGGTTGGCGTGG |
| | | | TAGTCGTGCTCGCTAACGCC |
| 296 | HSV-2 gC | Version | ATGGCTCTGGGCAGAGTTGGACTGGCTGTTG |
| | | 2.3 | GACTGTGGGGACTGCTGTGGGTTGGAGTGGT |
| | | | GGTGGTGCTGGCTAATGCT |
| 139 | HSV-2 gE | Version 2 | ATGGCACGGGGAGCCGGATTGGTGTTCTTTG |
| | | | TGGGCGTGTGGGTGGTGAGCTGCTTGGCAGC |
| | | | CGCACCA |
| 297 | HSV-2 gE | Version | ATGGCACGGGGCGCAGGTTTGGTCTTTTTCG |
| | | 2.1 | TGGGCGTGTGGGTGGTGAGCTGCTTGGCAGC |
| | | | CGCACCA |
| 298 | HSV-2 gE | Version | ATGGCGAGAGGAGCCGGACTCGTGTTCTTTG |
| | | 2.2 | TGGGAGTCTGGGTTGTGAGCTGCCTGGCAGC |
| | | | AGCTCCA |
| 299 | HSV-2 gE | Version | ATGGCGAGAGGAGCCGGGCTCGTGTTCTTTG |
| | | 2.3 | TGGGCGTATGGGTCGTTTCCTGCCTGGCTGC |
| | | | CGCACCC |
| 300 | HSV-2 gE − LVVVP | Version 2 | ATGGCGAGAGGAGCCGGACTCGTGTTCTTTG |
| | | | TGGGAGTCTGGGTTGTGAGCTGCCTGGTAGT |
| | | | AGTTCCA |
| 333 | HSV-1 gD | Version 1 | ATGGGCGGAGCTGCTGCTAGACTGGGAGCC |
| | | | GTGATCCTGTTCGTGGTTATCGTGGGACTGC |
| | | | ATGGCGTGCGGGGC |
| 337 | HSV-1 gD | Version 2 | ATGGGAGGAGCAGCTGCCAGACTCGGTGCC |
| | | | GTGATCCTGTTCGTGGTCATTGTTGGCCTGC |
| | | | ACGGGGTAAGGGGC |
| 338 | HSV-1 gD | Version 3 | ATGGGAGGAGCTGCTGCTAGATTGGGAGCT |
| | | | GTGATTCTGTTTGTGGTGATTGTGGGACTGC |
| | | | ATGGAGTGAGAGGA |
| 339 | HSV-1 gD | Version 4 | ATGGGCGGCGCAGCCGCTAGACTGGGTGCA |
| | | | GTCATCCTCTTTGTGGTGATCGTGGGGCTGC |
| | | | ATGGGGTCAGAGGG |
| 363 | HSV-1 gD | Version | ATGGGCGGAGCTGCGGCTCGCCTCGGAGCC |
| | | 4.1 | GTCATTCTGTTCGTGGTGATCGTGGGTCTGC |
| | | | ATGGGGTCAGAGGA |
| 218 | HSV-1 gD − KY | Version 1 | ATGGGCGGAGCTGCTGCTAGACTGGGAGCC |
| | | | GTGATCCTGTTCGTGGTTATCGTGGGACTGC |
| | | | ATGGCGTGCGGGGCAAGTAT |
| 219 | HSV-1 gD − KY | Version 2 | ATGGGAGGAGCAGCTGCCAGACTCGGTGCC |
| | | | GTGATCCTGTTCGTGGTCATTGTTGGCCTGC |
| | | | ACGGGGTAAGGGGCAAGTAC |
| 220 | HSV-1 gD − KY | Version 3 | ATGGGAGGAGCTGCTGCTAGATTGGGAGCT |
| | | | GTGATTCTGTTTGTGGTGATTGTGGGACTGC |
| | | | ATGGAGTGAGAGGAAAATAC |
| 221 | HSV-1 gD − KY | Version 4 | ATGGGCGGCGCAGCCGCTAGACTGGGTGCA |
| | | | GTCATCCTCTTTGTGGTGATCGTGGGGCTGC |
| | | | ATGGGGTCAGAGGGAAGTAT |
| 222 | HSV-1 gD − KY | Version | ATGGGCGGAGCTGCGGCTCGCCTCGGAGCC |
| | | 4.1 | GTCATTCTGTTCGTGGTGATCGTGGGTCTGC |
| | | | ATGGGGTCAGAGGAAAGTAC |
| 223 | HSV-1 gB − AP | Version 1 | ATGCATCAGGGCGCTCCATCTTGGGGTAGAC |
| | | | GTTGGTTCGTTGTGTGGGCCCTGCTGGGACT |
| | | | GACACTGGGAGTTCTGGTTGCCTCTGCTGCT |
| | | | CCT |
| 224 | HSV-1 gB − AP | Version 2 | ATGCACCAGGGTGCCCCTTCCTGGGGCAGA |
| | | | AGGTGGTTCGTGGTGTGGGCCCTTCTGGGGC |
| | | | TGACCCTCGGAGTCCTGGTTGCGAGCGCAGC |
| | | | TCCC |
| 225 | HSV-1 gB − AP | Version 3 | ATGCACCAGGGAGCACCTTCTTGGGGAAGA |
| | | | AGATGGTTTGTGGTGTGGGCTCTGCTGGGAC |
| | | | TGACCCTGGGAGTGCTGGTGGCTTCTGCTGC |
| | | | TCCT |
| 226 | HSV-1 gB − AP | Version 4 | ATGCATCAAGGCGCACCCAGTTGGGGCAGA |
| | | | CGGTGGTTCGTAGTGTGGGCCTTGCTGGGAC |
| | | | TGACTCTCGGTGTCCTCGTTGCGTCTGCTGC |
| | | | ACCG |
| 364 | HSV-1 gB | Version 1 | ATGCATCAGGGCGCTCCATCTTGGGGTAGAC |
| | | | GTTGGTTCGTTGTGTGGGCCCTGCTGGGACT |
| | | | GACACTGGGAGTTCTGGTTGCCTCTGCT |
| 365 | HSV-1 gB | Version 2 | ATGCACCAGGGTGCCCCTTCCTGGGGCAGA |
| | | | AGGTGGTTCGTGGTGTGGGCCCTTCTGGGGC |
| | | | TGACCCTCGGAGTCCTGGTTGCGAGCGCA |
| 366 | HSV-1 gB | Version 3 | ATGCACCAGGGAGCACCTTCTTGGGGAAGA |
| | | | AGATGGTTTGTGGTGTGGGCTCTGCTGGGAC |
| | | | TGACCCTGGGAGTGCTGGTGGCTTCTGCT |
| 367 | HSV-1 gB | Version 4 | ATGCATCAAGGCGCACCCAGTTGGGGCAGA |
| | | | CGGTGGTTCGTAGTGTGGGCCTTGCTGGGAC |
| | | | TGACTCTCGGTGTCCTCGTTGCGTCTGCT |
| 227 | HSV-2 gI + L | Version 1 | ATGCCTGGCAGATCTCTGCAAGGACTGGCCA |
| | | | TCCTCGGACTGTGGGTTTGCGCAACAGGACT |
| | | | G |
| 228 | HSV-2 gI + L | Version 2 | ATGCCCGGCAGAAGCCTCCAGGGACTGGCT |
| | | | ATCCTGGGGCTGTGGGTGTGCGCCACCGGTC |
| | | | TT |
| 229 | HSV-2 gI + L | Version 3 | ATGCCTGGAAGATCTCTCCAGGGACTGGCA |
| | | | ATCCTGGGACTGTGGGTGTGTGCAACAGGA |
| | | | CTG |
| 230 | HSV-2 gI + L | Version 4 | ATGCCTGGGAGAAGCCTGCAAGGGCTCGCA |
| | | | ATCTTGGGCCTGTGGGTTTGTGCCACAGGCT |
| | | | TG |
| 231 | HSV-2 gE − RTS | Version 1 | ATGGCTAGAGGTGCCGGCCTGGTGTTCTTTG |
| | | | TTGGCGTGTGGGTCGTGTCCTGTCTGGCCGC |
| | | | TGCTCCTAGAACATCT |
| 232 | HSV-2 gE − RTS | Version 2 | ATGGCGAGAGGAGCCGGGCTCGTGTTCTTTG |
| | | | TGGGCGTATGGGTCGTTTCCTGCCTGGCTGC |
| | | | CGCACCCAGGACCAGC |
| 233 | HSV-2 gE − RTS | Version 3 | ATGGCAAGAGGAGCAGGACTGGTGTTTTTC |
| | | | GTGGGAGTTTGGGTGGTGTCTTGCCTGGCTG |
| | | | CTGCTCCAAGAACCTCT |
| 234 | HSV-2 gE − RTS | Version 4 | ATGGCGAGAGGAGCCGGACTCGTGTTCTTTG |
| | | | TGGGAGTCTGGGTTGTGAGCTGCCTGGCAGC |
| | | | AGCTCCACGCACTAGC |
| 235 | EboZ − IP | Version 1 | ATGGGAGTGACCGGCATTCTCCAGCTGCCTC |
| | | | GGGACAGATTCAAGCGGACCAGCTTCTTCCT |
| | | | GTGGGTCATCATCCTGTTCCAGCGGACCTTC |
| | | | AGCATCCCC |
| 236 | EboZ − IP | Version 2 | ATGGGAGTGACTGGCATACTCCAGCTTCCTA |
| | | | GAGACAGGTTTAAGCGCACATCCTTCTTTCT |
| | | | GTGGGTCATCATCCTGTTCCAACGGACCTTC |
| | | | AGCATTCCC |
| 237 | EboZ − IP | Version 3 | ATGGGAGTAACCGGAATTCTCCAGCTGCCA |
| | | | AGAGATCGATTCAAAAGAACATCATTTTTCC |
| | | | TTTGGGTAATTATTCTGTTTCAGAGAACATT |
| | | | TTCCATCCCT |
| 238 | EboZ − IP | Version 4 | ATGGGAGTTACCGGAATTCTGCAATTGCCCA |
| | | | GGGATCGGTTCAAGCGCACATCCTTCTTCCT |
| | | | GTGGGTCATCATCCTCTTTCAGCGTACTTTCT |
| | | | CCATACCC |

TABLE 9
Example Ribonucleic Acid Sequences
encoding secretory signals

| SEQ | | | |
|---|---|---|---|
| ID | Secretory | | |
| NO: | signal | Versions | Sequence (Nucleotide) |
| 50 | IL2 | Wild type | AUGCGCAUGCAGCUGCUGCUGCUGAUCGCC |
| | | | CUGUCCCUGGCCCUGGUGACCAACUCC |
| 51 | IL2 | Version 1 | AUGAGAAUGCAGCUGCUGCUCCUGAUCGCC |
| | | | CUGUCUCUGGCCCUGGUCACCAAU |
| 52 | IL2 | Version 2 | AUGCGCAUGCAACUGCUCCUGCUGAUUGCG |
| | | | UUGAGCCUUGCCCUGGUGACCAACAGC |
| 53 | IL2 | Version 3 | AUGAGAAUGCAGCUGCUGCUGCUGAUCGCC |
| | | | CUGUCCCUGGCCCUGGUGACCAACUCC |
| 54 | HSV-2 gD | Wild type | AUGGGCCGCCUGACCUCCGGCGUGGGCACC |
| | | | GCCGCCCUGCUGGUGGUGGCCGUGGGCCUG |
| | | | CGCGUGGUGUGCGCC |
| 55 | HSV-2 gD | Version 1 | AUGGGCAGACUGACAUCUGGCGUGGGAAC |
| | | | AGCUGCUCUGCUGGUGGUUGCUGUGGGCC |
| | | | UGAGAGUCGUGUGUGCC |
| 56 | HSV-2 gD | Version 2 | AUGGGGAGACUCACAUCAGGCGUAGGAAC |
| | | | CGCUGCCCUGUUGGUCGUGGCCGUUGGUCU |
| | | | GAGAGUUGUGUGUGCC |
| 57 | HSV-2 gD | Version 3 | AUGGGCAGACUGACCUCCGGCGUGGGCACC |
| | | | GCCGCCCUGCUGGUGGUGGCCGUGGGCCUG |
| | | | AGAGUGGUGUGCGCC |
| 58 | HSV-2 gD − KYA | Wild type | AUGGGCCGCCUGACCUCCGGCGUGGGCACC |
| | | | GCCGCCCUGCUGGUGGUGGCCGUGGGCCUG |
| | | | CGCGUGGUGUGCGCCAAGUACGCC |
| 111 | HSV-2 gD − KYA | Version 1 | AUGGGCAGACUGACAUCUGGCGUGGGAAC |
| | | | AGCUGCUCUGCUGGUGGUUGCUGUGGGCC |
| | | | UGAGAGUCGUGUGUGCCAAAUACGCC |
| 59 | HSV-2 gD − KYA | Version 2 | AUGGGGAGACUCACAUCAGGCGUAGGAAC |
| | | | CGCUGCCCUGUUGGUCGUGGCCGUUGGUCU |
| | | | GAGAGUUGUGUGUGCCAAAUAUGCU |
| 60 | HSV-2 gD − KYA | Version 3 | AUGGGGAGACUCACAUCAGGCGUAGGAAC |
| | | | CGCUGCCCUGUUGGUCGUGGCCGUUGGUCU |
| | | | GAGAGUUGUGUGUGCCAAAUAUGCU |
| 368 | HSV-2 gD − KYAL | Version 2 | AUGGGGAGACUCACAUCAGGCGUAGGAAC |
| | | | CGCUGCCCUGUUGGUCGUGGCCGUUGGUCU |
| | | | GAGAGUUGUGUGUGCCAAAUAUGCUCUG |
| 369 | HSV-2 gD − KYAL | Version 3 | AUGGGCAGACUGACCUCCGGCGUGGGCACC |
| | | | GCCGCCCUGCUGGUGGUGGCCGUGGGCCUG |
| | | | AGAGUGGUGUGCGCCAAAUACGCCCUG |
| 61 | HSV-2 gD − KYALA | Wild type | AUGGGCCGCCUGACCUCCGGCGUGGGCACC |
| | | | GCCGCCCUGCUGGUGGUGGCCGUGGGCCUG |
| | | | CGCGUGGUGUGCGCCAAGUACGCCCUGGCC |
| 62 | HSV-2 gD − KYALA | Version 1 | AUGGGCAGACUGACAUCUGGCGUGGGAAC |
| | | | AGCUGCUCUGCUGGUGGUUGCUGUGGGCC |
| | | | UGAGAGUCGUGUGUGCCAAAUACGCCCUG |
| | | | GCC |
| 63 | HSV-2 gD − KYALA | Version 2 | AUGGGGAGACUCACAUCAGGCGUAGGAAC |
| | | | CGCUGCCCUGUUGGUCGUGGCCGUUGGUCU |
| | | | GAGAGUUGUGUGUGCCAAAUAUGCUCUG |
| 301 | HSV-2 gD − KYALA | Version 2.1 | AUGGGGAGACUCACAUCCGGAGUGGGCAC |
| | | | UGCUGCCCUGUUGGUCGUGGCCGUUGGUCU |
| | | | GAGAGUUGUGUGUGCCAAAUAUGCUCUGG |
| | | | CA |
| 64 | HSV-2 gD − KYALA | Version 3 | AUGGGCAGACUGACCUCCGGCGUGGGCACC |
| | | | GCCGCCCUGCUGGUGGUGGCCGUGGGCCUG |
| | | | AGAGUGGUGUGCGCCAAAUACGCCCUG |
| 140 | HSV-2 gC | Version 2 | AUGGCCUUGGGGAGAGUGGGCCUUGCAGU |
| | | | GGGUCUGUGGGGACUGCUCUGGGUUGGCG |
| | | | UAGUCGUCGUGCUGGCUAACGCA |
| 302 | HSV-2 gC | Version 2.1 | AUGGCACUAGGGAGAGUGGGAUUAGCUGU |
| | | | GGGUCUGUGGGGACUGCUCUGGGUAGGAG |
| | | | UCGUCGUCGUGCUGGCUAACGCA |
| 303 | HSV-2 gC | Version 2.2 | AUGGCCUUGGGGAGAGUCGGCCUUGCAGU |
| | | | GGGACUGUGGGGUCUGCUGUGGGUUGGCG |
| | | | UGGUAGUCGUGCUCGCUAACGCC |
| 304 | HSV-2 gC | Version 2.3 | AUGGCUCUGGGCAGAGUUGGACUGGCUGU |
| | | | UGGACUGUGGGGACUGCUGUGGGUUGGAG |
| | | | UGGUGGUGGUGCUGGCUAAUGCU |
| 141 | HSV-2 gE | Version 2 | AUGGCACGGGGAGCCGGAUUGGUGUUCUU |
| | | | UGUGGGCGUGUGGGUGGUGAGCUGCUUGG |
| | | | CAGCCGCACCA |
| 305 | HSV-2 gE | Version 2.1 | AUGGCACGGGGCGCAGGUUUGGUCUUUUU |
| | | | CGUGGGCGUGUGGGUGGUGAGCUGCUUGG |
| | | | CAGCCGCACCA |
| 306 | HSV-2 gE | Version 2.2 | AUGGCGAGAGGAGCCGGACUCGUGUUCUU |
| | | | UGUGGGAGUCUGGGUUGUGAGCUGCCUGG |
| | | | CAGCAGCUCCA |
| 307 | HSV-2 gE | Version 2.3 | AUGGCGAGAGGAGCCGGGCUCGUGUUCUU |
| | | | UGUGGGCGUAUGGGUCGUUUCCUGCCUGG |
| | | | CUGCCGCACCC |
| 308 | HSV-2 gE − LVVVP | Version 2 | AUGGCGAGAGGAGCCGGACUCGUGUUCUU |
| | | | UGUGGGAGUCUGGGUUGUGAGCUGCCUGG |
| | | | UAGUAGUUCCA |
| 370 | HSV-1 gD | Version 1 | AUGGGCGGAGCUGCUGCUAGACUGGGAGC |
| | | | CGUGAUCCUGUUCGUGGUUAUCGUGGGAC |
| | | | UGCAUGGCGUGCGGGGC |
| 371 | HSV-1 gD | Version 2 | AUGGGAGGAGCAGCUGCCAGACUCGGUGCC |
| | | | GUGAUCCUGUUCGUGGUCAUUGUUGGCCU |
| | | | GCACGGGGUAAGGGGC |
| 372 | HSV-1 gD | Version 3 | AUGGGAGGAGCUGCUGCUAGAUUGGGAGC |
| | | | UGUGAUUCUGUUUGUGGUGAUUGUGGGAC |
| | | | UGCAUGGAGUGAGAGGA |
| 373 | HSV-1 gD | Version 4 | AUGGGCGGCGCAGCCGCUAGACUGGGUGCA |
| | | | GUCAUCCUCUUUGUGGUGAUCGUGGGGCU |
| | | | GCAUGGGGUCAGAGGG |
| 374 | HSV-1 gD | Version 4.1 | AUGGGCGGAGCUGCGGCUCGCCUCGGAGCC |
| | | | GUCAUUCUGUUCGUGGUGAUCGUGGGUCU |
| | | | GCAUGGGGUCAGAGGA |
| 239 | HSV-1 gD − KY | Version 1 | AUGGGCGGAGCUGCUGCUAGACUGGGAGC |
| | | | CGUGAUCCUGUUCGUGGUUAUCGUGGGAC |
| | | | UGCAUGGCGUGCGGGGCAAGUAU |
| 240 | HSV-1 gD − KY | Version 2 | AUGGGAGGAGCAGCUGCCAGACUCGGUGCC |
| | | | GUGAUCCUGUUCGUGGUCAUUGUUGGCCU |
| | | | GCACGGGGUAAGGGGCAAGUAC |
| 241 | HSV-1 gD − KY | Version 3 | AUGGGAGGAGCUGCUGCUAGAUUGGGAGC |
| | | | UGUGAUUCUGUUUGUGGUGAUUGUGGGAC |
| | | | UGCAUGGAGUGAGAGGAAAAUAC |
| 242 | HSV-1 gD − KY | Version 4 | AUGGGCGGCGCAGCCGCUAGACUGGGUGCA |
| | | | GUCAUCCUCUUUGUGGUGAUCGUGGGGCU |
| | | | GCAUGGGGUCAGAGGGAAGUAU |
| 243 | HSV-1 gD − KY | Version 4.1 | AUGGGCGGAGCUGCGGCUCGCCUCGGAGCC |
| | | | GUCAUUCUGUUCGUGGUGAUCGUGGGUCU |
| | | | GCAUGGGGUCAGAGGAAAGUAC |
| 244 | HSV-1 gB − AP | Version 1 | AUGCAUCAGGGCGCUCCAUCUUGGGGUAG |
| | | | ACGUUGGUUCGUUGUGUGGGCCCUGCUGG |
| | | | GACUGACACUGGGAGUUCUGGUUGCCUCU |
| | | | GCUGCUCCU |
| 245 | HSV-1 gB − AP | Version 2 | AUGCACCAGGGUGCCCCUUCCUGGGGCAGA |
| | | | AGGUGGUUCGUGGUGUGGGCCCUUCUGGG |
| | | | GCUGACCCUCGGAGUCCUGGUUGCGAGCGC |
| | | | AGCUCCC |
| 246 | HSV-1 gB − AP | Version 3 | AUGCACCAGGGAGCACCUUCUUGGGGAAG |
| | | | AAGAUGGUUUGUGGUGUGGGCUCUGCUGG |
| | | | GACUGACCCUGGGAGUGCUGGUGGCUUCU |
| | | | GCUGCUCCU |
| 247 | HSV-1 gB − AP | Version 4 | AUGCAUCAAGGCGCACCCAGUUGGGGCAGA |
| | | | CGGUGGUUCGUAGUGUGGGCCUUGCUGGG |
| | | | ACUGACUCUCGGUGUCCUCGUUGCGUCUGC |
| | | | UGCACCG |
| 375 | HSV-1 gB | Version 1 | AUGCAUCAGGGCGCUCCAUCUUGGGGUAG |
| | | | ACGUUGGUUCGUUGUGUGGGCCCUGCUGG |
| | | | GACUGACACUGGGAGUUCUGGUUGCCUCU |
| | | | GCU |
| 376 | HSV-1 gB | Version 2 | AUGCACCAGGGUGCCCCUUCCUGGGGCAGA |
| | | | AGGUGGUUCGUGGUGUGGGCCCUUCUGGG |
| | | | GCUGACCCUCGGAGUCCUGGUUGCGAGCGC |
| | | | A |
| 377 | HSV-1 gB | Version 3 | AUGCACCAGGGAGCACCUUCUUGGGGAAG |
| | | | AAGAUGGUUUGUGGUGUGGGCUCUGCUGG |
| | | | GACUGACCCUGGGAGUGCUGGUGGCUUCU |
| | | | GCU |
| 378 | HSV-1 gB | Version 4 | AUGCAUCAAGGCGCACCCAGUUGGGGCAGA |
| | | | CGGUGGUUCGUAGUGUGGGCCUUGCUGGG |
| | | | ACUGACUCUCGGUGUCCUCGUUGCGUCUGC |
| | | | U |
| 248 | HSV-2 gI + L | Version 1 | AUGCCUGGCAGAUCUCUGCAAGGACUGGCC |
| | | | AUCCUCGGACUGUGGGUUUGCGCAACAGG |
| | | | ACUG |
| 249 | HSV-2 gI + L | Version 2 | AUGCCCGGCAGAAGCCUCCAGGGACUGGCU |
| | | | AUCCUGGGGCUGUGGGUGUGCGCCACCGGU |
| | | | CUU |
| 250 | HSV-2 gI + L | Version 3 | AUGCCUGGAAGAUCUCUCCAGGGACUGGCA |
| | | | AUCCUGGGACUGUGGGUGUGUGCAACAGG |
| | | | ACUG |
| 251 | HSV-2 gI + L | Version 4 | AUGCCUGGGAGAAGCCUGCAAGGGCUCGCA |
| | | | AUCUUGGGCCUGUGGGUUUGUGCCACAGG |
| | | | CUUG |
| 252 | HSV-2 gE − RTS | Version 1 | AUGGCUAGAGGUGCCGGCCUGGUGUUCUU |
| | | | UGUUGGCGUGUGGGUCGUGUCCUGUCUGG |
| | | | CCGCUGCUCCUAGAACAUCU |
| 253 | HSV-2 gE − RTS | Version 2 | AUGGCGAGAGGAGCCGGGCUCGUGUUCUU |
| | | | UGUGGGCGUAUGGGUCGUUUCCUGCCUGG |
| | | | CUGCCGCACCCAGGACCAGC |
| 254 | HSV-2 gE − RTS | Version 3 | AUGGCAAGAGGAGCAGGACUGGUGUUUUU |
| | | | CGUGGGAGUUUGGGUGGUGUCUUGCCUGG |
| | | | CUGCUGCUCCAAGAACCUCU |
| 255 | HSV-2 gE − RTS | Version 4 | AUGGCGAGAGGAGCCGGACUCGUGUUCUU |
| | | | UGUGGGAGUCUGGGUUGUGAGCUGCCUGG |
| | | | CAGCAGCUCCACGCACUAGC |
| 256 | EboZ − IP | Version 1 | AUGGGAGUGACCGGCAUUCUCCAGCUGCCU |
| | | | CGGGACAGAUUCAAGCGGACCAGCUUCUUC |
| | | | CUGUGGGUCAUCAUCCUGUUCCAGCGGACC |
| | | | UUCAGCAUCCCC |
| 257 | EboZ − IP | Version 2 | AUGGGAGUGACUGGCAUACUCCAGCUUCCU |
| | | | AGAGACAGGUUUAAGCGCACAUCCUUCUU |
| | | | UCUGUGGGUCAUCAUCCUGUUCCAACGGAC |
| | | | CUUCAGCAUUCCC |
| 258 | EboZ − IP | Version 3 | AUGGGAGUAACCGGAAUUCUCCAGCUGCCA |
| | | | AGAGAUCGAUUCAAAAGAACAUCAUUUUU |
| | | | CCUUUGGGUAAUUAUUCUGUUUCAGAGAA |
| | | | CAUUUUCCAUCCCU |
| 259 | EboZ − IP | Version 4 | AUGGGAGUUACCGGAAUUCUGCAAUUGCC |
| | | | CAGGGAUCGGUUCAAGCGCACAUCCUUCUU |
| | | | CCUGUGGGUCAUCAUCCUCUUUCAGCGUAC |
| | | | UUUCUCCAUACCC |

### C. Certain Example Antigen-Secretory Signal Combinations

In some embodiments, a polyribonucleotide encodes a polypeptide, wherein the polypeptide comprises an HSV-2 glycoprotein antigen or antigenic fragment thereof and a secretory signal. In some embodiments, a polyribonucleotide encodes a polypeptide, wherein the polypeptide comprises an HSV-2 glycoprotein antigen and a secretory signal.

Example polyribonucleotide constructs encoding a gC, gD, or gE antigen as described herein are provided in Table 10 below.

TABLE 10
Example Polyribonucleotide Constructs

| | Secretory | | | Amino acid | RNA SEQ |
|---|---|---|---|---|---|
| Construct | signal | Antigen | Additional modifications | SEQ ID NO | ID NO |
| 597 | IL2 | gC | | 65 | 104 |
| 598 | gD2 | gD | | 70 | 116 |
| 599 | IL2 | gE | | 73 | 121 |
| 600 | IL2 | gC | Codon optimized (Version 2) | 65 | 106 |
| 1601 | gD2 | gD | Codon optimized (Version 2) | 70 | 118 |
| 1602 | IL2 | gE | Codon optimized (Version 2) | 73 | 123 |
| 1658 | gD2 | gC | Codon optimized (Version 2); | 67 | 109 |
| | | | KYA secretory signal extension; | | |
| | | | ΔEAM mutation | | |
| 1659 | gD2 | gD | Codon optimized (Version 2); | 70 | 119 |
| | | | ΔEAM mutation | | |
| 1660 | gD2 | gE | Codon optimized (Version 2); | 75 | 127 |
| | | | KYA secretory signal extension; | | |
| | | | ΔEAM mutation | | |
| 1622 | gD2 | gC | KYA secretory signal extension | 67 | 108 |
| 1623 | gD2 | gE | KYA secretory signal extension | 75 | 125 |
| 1624 | gD2 | gC | Codon optimized (Version 2); | 67 | 110 |
| | | | KYA secretory signal extension; 4 | | |
| | | | point mutations relative to 1658 | | |
| 1625 | gD2 | gE | Codon optimized (Version 2); | 75 | 128 |
| | | | KYA secretory signal extension; 3 | | |
| | | | point mutations relative to 1660 | | |
| 1873 | IL2 | gC | Codon optimized (Version 1) | 65 | 105 |
| 1874 | gD2 | gD | Codon optimized (Version 1) | 70 | 117 |
| 1875 | IL2 | gE | Codon optimized (Version 1) | 73 | 122 |
| 1876 | IL2 | gC | Codon optimized (Version 3) | 65 | 107 |
| 1877 | gD2 | gD | Codon optimized (Version 3) | 70 | 120 |
| 1878 | IL2 | gE | Codon optimized (Version 3) | 73 | 124 |
| 1907 | gD2 | gC | Codon optimized (Version 2); | 68 | 113 |
| | | | KYALA secretory signal extension | | |
| 1908 | gD2 | gC | Codon optimized (Version 1); | 68 | 112 |
| | | | KYALA secretory signal extension | | |
| 1909 | gD2 | gC | Codon optimized (Version 3); | 68 | 114 |
| | | | KYALA secretory signal extension | | |
| 1910 | gC2 | gC | Codon optimized (Version 2); | 131 | 115 |
| 1911 | gD2 | gE | Codon optimized (Version 1); | 75 | 126 |
| | | | KYA secretory signal extension | | |
| 1912 | gD2 | gE | Codon optimized (Version 3); | 75 | 129 |
| | | | KYA secretory signal extension | | |
| 1913 | gE2 | gE | Codon optimized (Version 2); | 132 | 130 |
| 2140 | gD1 | gC | Codon optimized (Version 4); KY | 159 | 192 |
| | | | secretory signal extension | | |
| 2141 | gB1 | gC | Codon optimized (Version 4); AP | 160 | 196 |
| | | | secretory signal extension | | |
| 2138 | gE2 | gC | Codon optimized (Version 4); RTS | 162 | 204 |
| | | | secretory signal extension | | |
| 2139 | gI2 | gC2 | Codon optimized (Version 2) | 161 | 200 |
| | | | TGL secretory signal extension | | |
| 2142 | EboZ | gC | Codon optimized (Version 2); IP | 163 | 208 |
| | | | secretory signal extension | | |
| 2143 | gD1 | gE | Codon optimized (Version 4); KY | 164 | 212 |
| | | | secretory signal extension; 3 point | | |
| | | | mutations relative to 1602 | | |
| 2538 | gD1 | gC | Codon optimized (Version 2); KY | 159 | 190 |
| | | | secretory signal extension | | |
| 2541 | gB1 | gC | Codon optimized (Version 2); AP | 160 | 194 |
| | | | secretory signal extension | | |
| 2544 | gI2 | gC | Codon optimized (Version 2); TGL | 161 | 198 |
| | | | secretory signal extension | | |
| 2547 | gE2 | gC | Codon optimized (Version 2); RTS | 162 | 202 |
| | | | secretory signal extension | | |
| 2550 | EboZ | gC | Codon optimized (Version 2); IP | 163 | 206 |
| | | | secretory signal extension | | |
| 2553 | gD1 | gE | Codon optimized (Version 2); KY | 164 | 210 |
| | | | secretory signal extension; 3 point | | |
| | | | mutations relative to 1602 | | |
| 2537 | gD1 | gC | Codon optimized (Version 1); KY | 159 | 189 |
| | | | secretory signal extension | | |
| 2540 | gB1 | gC | Codon optimized (Version 1); AP | 160 | 193 |
| | | | secretory signal extension | | |
| 2543 | gI2 | gC | Codon optimized (Version 1); TGL | 161 | 197 |
| | | | secretory signal extension | | |
| 2546 | gE2 | gC | Codon optimized (Version 1); RTS | 162 | 201 |
| | | | secretory signal extension | | |
| 2549 | EboZ | gC | Codon optimized (Version 1); IP | 163 | 205 |
| | | | secretory signal extension | | |
| 2552 | gD1 | gE | Codon optimized (Version 1); KY | 164 | 209 |
| | | | secretory signal extension; 3 point | | |
| | | | mutations relative to 1602 | | |
| 2539 | gD1 | gC | Codon optimized (Version 3); KY | 159 | 191 |
| | | | secretory signal extension | | |
| 2542 | gB1 | gC | Codon optimized (Version 3); AP | 160 | 195 |
| | | | secretory signal extension | | |
| 2545 | gI2 | gC | Codon optimized (Version 3); TGL | 161 | 199 |
| | | | secretory signal extension | | |
| 2548 | gE2 | gC | Codon optimized (Version 3); RTS | 162 | 203 |
| | | | secretory signal extension | | |
| 2551 | EboZ | gC | Codon optimized (Version 3); IP | 163 | 207 |
| | | | secretory signal extension | | |
| 2554 | gD1 | gE | Codon optimized (Version 3); KY | 164 | 211 |
| | | | secretory signal extension; 3 point | | |
| | | | mutations relative to 1602 | | |
| 2790 | gE2 | gE | Codon optimized (Version 2); | 132 | 322 |
| | | | mutations to improve RNA | | |
| | | | secondary structure | | |
| 2788 | gE2 | gE | Codon optimized (Version 2) | 132 | 323 |
| 2792 | gE2 | gE | Codon optimized (Version 2) | 132 | 324 |
| 2791 | gE2 | gE | Codon optimized (Version 2); | 327 | 325 |
| | | | LVVV secretory signal extension; | | |
| | | | mutations to avoid secretion | | |
| 2789 | — | gE | Codon optimized (Version 2) | 328 | 326 |
| 2786 | gC2 | gC | Codon optimized (Version 2); | 131 | 319 |
| | | | mutations to improve RNA | | |
| | | | secondary structure | | |
| 2787 | gD2 | gC | Codon optimized (Version 2); | 68 | 318 |
| | | | KYALA secretory signal | | |
| | | | extension; mutations to improve | | |
| | | | RNA secondary structure | | |
| 2785 | gC2 | gC | Codon optimized (Version 2) | 131 | 320 |
| 2784 | gC2 | gC | Codon optimized (Version 2) | 131 | 321 |
| 3215 | gD1 | gC | Codon optimized (Version 2); KY | 159 | 348 |
| | | | secretory signal extension; ΔEAM | | |
| | | | deletion | | |
| 3216 | gB1 | gC | Codon optimized (Version 2); AP | 160 | 350 |
| | | | secretory signal extension; ΔEAM | | |
| | | | deletion | | |
| 3217 | gE2 | gC | Codon optimized (Version 2); RTS | 162 | 351 |
| | | | secretory signal extension; ΔEAM | | |
| | | | deletion | | |
| 3233 | gD1 | gC | Codon optimized (Version 2); KY | 159 | 349 |
| | | | secretory signal extension; ΔEAM | | |
| | | | deletion | | |
| 3234 | gD2 | gD | Codon optimized (Version 2); | 70 | 352 |
| | | | second stop codon | | |
| 3235 | gD2 | gE | Codon optimized (Version 2); | 75 | 353 |
| | | | KYA secretory signal extension; | | |
| | | | second stop codon | | |

In some embodiments, a polypeptide as described herein (or encoded by a polyribonucleotide as described herein) comprises an HSV-2 gC antigen and a secretory signal. Example combinations of an HSV-2 gC antigen and secretory signal are provided in Table 11 below, along with example corresponding amino acid sequences.

In some embodiments, a polypeptide as described herein (or encoded by a polyribonucleotide as described herein) comprises an HSV-2 gD antigen and a secretory signal. Example combinations of an HSV-2 gD antigen and secretory signal are provided in Table 11 below, along with example corresponding amino acid sequences.

In some embodiments, a polypeptide as described herein (or encoded by a polyribonucleotide as described herein) comprises an HSV-2 gE antigen and a secretory signal. Example combinations of an HSV-2 gE antigen and secretory signal are provided in Table 11 below, along with example corresponding amino acid sequences. Exemplary nucleotide sequences are provided in Table 12 and Table 13.

TABLE 11
Example secretory signals and
HSV-2 glycoproteins Amino Acid Sequences

| SEQ | | | |
|---|---|---|---|
| ID | Secretory | Antigen/ | |
| NO: | signal | OFR | Sequence (Amino acids) |
| 65 | IL2 | HSV-2 gC | MRMQLLLLIALSLALVTNSASPGRTITVGPRGNAS |
| | | (27-426) | NAAPSASPRNASAPRTTPTPPQPRKATKSKASTAKPA |
| | | | PPPKTGPPKTSSEPVRCNRHDPLARYGSRVQIRCRFP |
| | | | NSTRTEFRLQIWRYATATDAEIGTAPSLEEVMVNVS |
| | | | APPGGQLVYDSAPNRTDPHVIWAEGAGPGASPRLYS |
| | | | VVGPLGRQRLIIEELTLETQGMYYWVWGRTDRPSA |
| | | | YGTWVRVRVFRPPSLTIHPHAVLEGQPFKATCTAAT |
| | | | YYPGNRAEFVWFEDGRRVFDPAQIHTQTQENPDGFS |
| | | | TVSTVTSAAVGGQGPPRTFTCQLTWHRDSVSFSRRN |
| | | | ASGTASVLPRPTITMEFTGDHAVCTAGCVPEGVTFA |
| | | | WFLGDDSSPAEKVAVASQTSCGRPGTATIRSTLPVS |
| | | | YEQTEYICRLAGYPDGIPVLEHH |
| 66 | HSV-2 gD | HSV-2 gC | MGRLTSGVGTAALLVVAVGLRVVCAASPGRTITV |
| | | (27-426) | GPRGNASNAAPSASPRNASAPRTTPTPPQPRKATKS |
| | | | KASTAKPAPPPKTGPPKTSSEPVRCNRHDPLARYGS |
| | | | RVQIRCRFPNSTRTEFRLQIWRYATATDAEIGTAPSL |
| | | | EEVMVNVSAPPGGQLVYDSAPNRTDPHVIWAEGAG |
| | | | PGASPRLYSVVGPLGRQRLIIEELTLETQGMYYWVW |
| | | | GRTDRPSAYGTWVRVRVFRPPSLTIHPHAVLEGQPF |
| | | | KATCTAATYYPGNRAEFVWFEDGRRVFDPAQIHTQ |
| | | | TQENPDGFSTVSTVTSAAVGGQGPPRTFTCQLTWHR |
| | | | DSVSFSRRNASGTASVLPRPTITMEFTGDHAVCTAG |
| | | | CVPEGVTFAWFLGDDSSPAEKVAVASQTSCGRPGT |
| | | | ATIRSTLPVSYEQTEYICRLAGYPDGIPVLEHH |
| 67 | HSV-2 | HSV-2 gC | MGRLTSGVGTAALLVVAVGLRVVCAKYAASPGR |
| | gD − KYA | (28-426) | TITVGPRGNASNAAPSASPRNASAPRTTPTPPQPRKA |
| | | | TKSKASTAKPAPPPKTGPPKTSSEPVRCNRHDPLARY |
| | | | GSRVQIRCRFPNSTRTEFRLQIWRYATATDAEIGTAP |
| | | | SLEEVMVNVSAPPGGQLVYDSAPNRTDPHVIWAEG |
| | | | AGPGASPRLYSVVGPLGRQRLIIEELTLETQGMYYW |
| | | | VWGRTDRPSAYGTWVRVRVFRPPSLTIHPHAVLEG |
| | | | QPFKATCTAATYYPGNRAEFVWFEDGRRVFDPAQI |
| | | | HTQTQENPDGFSTVSTVTSAAVGGQGPPRTFTCQLT |
| | | | WHRDSVSFSRRNASGTASVLPRPTITMEFTGDHAVC |
| | | | TAGCVPEGVTFAWFLGDDSSPAEKVAVASQTSCGR |
| | | | PGTATIRSTLPVSYEQTEYICRLAGYPDGIPVLEHH |
| 68 | HSV-2 | HSV-2 gC | MGRLTSGVGTAALLVVAVGLRVVCAKYALASPG |
| | gD − KYALA | (28-426) | RTITVGPRGNASNAAPSASPRNASAPRTTPTPPQPRK |
| | | | ATKSKASTAKPAPPPKTGPPKTSSEPVRCNRHDPLAR |
| | | | YGSRVQIRCRFPNSTRTEFRLQIWRYATATDAEIGTA |
| | | | PSLEEVMVNVSAPPGGQLVYDSAPNRTDPHVIWAE |
| | | | GAGPGASPRLYSVVGPLGRQRLIIEELTLETQGMYY |
| | | | WVWGRTDRPSAYGTWVRVRVFRPPSLTIHPHAVLE |
| | | | GQPFKATCTAATYYPGNRAEFVWFEDGRRVFDPAQ |
| | | | IHTQTQENPDGFSTVSTVTSAAVGGQGPPRTFTCQLT |
| | | | WHRDSVSFSRRNASGTASVLPRPTITMEFTGDHAVC |
| | | | TAGCVPEGVTFAWFLGDDSSPAEKVAVASQTSCGR |
| | | | PGTATIRSTLPVSYEQTEYICRLAGYPDGIPVLEHH |
| 131 | HSV-2 gC | HSV-2 gC | MALGRVGLAVGLWGLLWVGVVVVLANASPGRTI |
| | | (28-426) | TVGPRGNASNAAPSASPRNASAPRTTPTPPQPRKAT |
| | | | KSKASTAKPAPPPKTGPPKTSSEPVRCNRHDPLARY |
| | | | GSRVQIRCRFPNSTRTEFRLQIWRYATATDAEIGTAP |
| | | | SLEEVMVNVSAPPGGQLVYDSAPNRTDPHVIWAEG |
| | | | AGPGASPRLYSVVGPLGRQRLIIEELTLETQGMYYW |
| | | | VWGRTDRPSAYGTWVRVRVFRPPSLTIHPHAVLEG |
| | | | QPFKATCTAATYYPGNRAEFVWFEDGRRVFDPAQI |
| | | | HTQTQENPDGFSTVSTVTSAAVGGQGPPRTFTCQLT |
| | | | WHRDSVSFSRRNASGTASVLPRPTITMEFTGDHAVC |
| | | | TAGCVPEGVTFAWFLGDDSSPAEKVAVASQTSCGR |
| | | | PGTATIRSTLPVSYEQTEYICRLAGYPDGIPVLEHH |
| 159 | HSV-1 | HSV-2 gC | MGGAAARLGAVILFVVIVGLHGVRGKYSPGRTIT |
| | gD − KY | (28-426) | VGPRGNASNAAPSASPRNASAPRTTPTPPQPRKATK |
| | | | SKASTAKPAPPPKTGPPKTSSEPVRCNRHDPLARYGS |
| | | | RVQIRCRFPNSTRTEFRLQIWRYATATDAEIGTAPSL |
| | | | EEVMVNVSAPPGGQLVYDSAPNRTDPHVIWAEGAG |
| | | | PGASPRLYSVVGPLGRQRLIIEELTLETQGMYYWVW |
| | | | GRTDRPSAYGTWVRVRVFRPPSLTIHPHAVLEGQPF |
| | | | KATCTAATYYPGNRAEFVWFEDGRRVFDPAQIHTQ |
| | | | TQENPDGFSTVSTVTSAAVGGQGPPRTFTCQLTWHR |
| | | | DSVSFSRRNASGTASVLPRPTITMEFTGDHAVCTAG |
| | | | CVPEGVTFAWFLGDDSSPAEKVAVASQTSCGRPGT |
| | | | ATIRSTLPVSYEQTEYICRLAGYPDGIPVLEHH |
| 160 | HSV-1 gB | HSV-2 gC | MHQGAPSWGRRWFVVWALLGLTLGVLVASAAP |
| | | (28-426) | SPGRTITVGPRGNASNAAPSASPRNASAPRTTPTPPQ |
| | | | PRKATKSKASTAKPAPPPKTGPPKTSSEPVRCNRHDP |
| | | | LARYGSRVQIRCRFPNSTRTEFRLQIWRYATATDAEI |
| | | | GTAPSLEEVMVNVSAPPGGQLVYDSAPNRTDPHVI |
| | | | WAEGAGPGASPRLYSVVGPLGRQRLIIEELTLETQG |
| | | | MYYWVWGRTDRPSAYGTWVRVRVFRPPSLTIHPHA |
| | | | VLEGQPFKATCTAATYYPGNRAEFVWFEDGRRVFD |
| | | | PAQIHTQTQENPDGFSTVSTVTSAAVGGQGPPRTFTC |
| | | | QLTWHRDSVSFSRRNASGTASVLPRPTITMEFTGDH |
| | | | AVCTAGCVPEGVTFAWFLGDDSSPAEKVAVASQTS |
| | | | CGRPGTATIRSTLPVSYEQTEYICRLAGYPDGIPVLE |
| | | | HH |
| 161 | HSV-2 | HSV-2 gC | MPGRSLQGLAILGLWVCATGLSPGRTITVGPRGN |
| | gI + L | (28-426) | ASNAAPSASPRNASAPRTTPTPPQPRKATKSKASTAK |
| | | | PAPPPKTGPPKTSSEPVRCNRHDPLARYGSRVQIRCR |
| | | | FPNSTRTEFRLQIWRYATATDAEIGTAPSLEEVMVN |
| | | | VSAPPGGQLVYDSAPNRTDPHVIWAEGAGPGASPRL |
| | | | YSVVGPLGRQRLIIEELTLETQGMYYWVWGRTDRPS |
| | | | AYGTWVRVRVFRPPSLTIHPHAVLEGQPFKATCTAA |
| | | | TYYPGNRAEFVWFEDGRRVFDPAQIHTQTQENPDGF |
| | | | STVSTVTSAAVGGQGPPRTFTCQLTWHRDSVSFSRR |
| | | | NASGTASVLPRPTITMEFTGDHAVCTAGCVPEGVTF |
| | | | AWFLGDDSSPAEKVAVASQTSCGRPGTATIRSTLPV |
| | | | SYEQTEYICRLAGYPDGIPVLEHH |
| 162 | HSV-2 | HSV-2 gC | MARGAGLVFFVGVWVVSCLAAAPRTSSPGRTITV |
| | gE + RTS | (28-426) | GPRGNASNAAPSASPRNASAPRTTPTPPQPRKATKS |
| | | | KASTAKPAPPPKTGPPKTSSEPVRCNRHDPLARYGS |
| | | | RVQIRCRFPNSTRTEFRLQIWRYATATDAEIGTAPSL |
| | | | EEVMVNVSAPPGGQLVYDSAPNRTDPHVIWAEGAG |
| | | | PGASPRLYSVVGPLGRQRLIIEELTLETQGMYYWVW |
| | | | GRTDRPSAYGTWVRVRVFRPPSLTIHPHAVLEGQPF |
| | | | KATCTAATYYPGNRAEFVWFEDGRRVFDPAQIHTQ |
| | | | TQENPDGFSTVSTVTSAAVGGQGPPRTFTCQLTWHR |
| | | | DSVSFSRRNASGTASVLPRPTITMEFTGDHAVCTAG |
| | | | CVPEGVTFAWFLGDDSSPAEKVAVASQTSCGRPGT |
| | | | ATIRSTLPVSYEQTEYICRLAGYPDGIPVLEHH |
| 163 | EboZ | HSV-2 gC | MGVTGILQLPRDRFKRTSFFLWVIILFQRTFSIPSP |
| | | (28-426) | GRTITVGPRGNASNAAPSASPRNASAPRTTPTPPQPR |
| | | | KATKSKASTAKPAPPPKTGPPKTSSEPVRCNRHDPL |
| | | | ARYGSRVQIRCRFPNSTRTEFRLQIWRYATATDAEIG |
| | | | TAPSLEEVMVNVSAPPGGQLVYDSAPNRTDPHVIW |
| | | | AEGAGPGASPRLYSVVGPLGRQRLIIEELTLETQGM |
| | | | YYWVWGRTDRPSAYGTWVRVRVFRPPSLTIHPHAV |
| | | | LEGQPFKATCTAATYYPGNRAEFVWFEDGRRVFDP |
| | | | AQIHTQTQENPDGFSTVSTVTSAAVGGQGPPRTFTC |
| | | | QLTWHRDSVSFSRRNASGTASVLPRPTITMEFTGDH |
| | | | AVCTAGCVPEGVTFAWFLGDDSSPAEKVAVASQTS |
| | | | CGRPGTATIRSTLPVSYEQTEYICRLAGYPDGIPVLE |
| | | | HH |
| 69 | IL2 | HSV-2 gD | MRMQLLLLIALSLALVTNSADPSLKMADPNRFRG |
| | | (30-331) | KNLPVLDQLTDPPGVKRVYHIQPSLEDPFQPPSIPITV |
| | | | YYAVLERACRSVLLHAPSEAPQIVRGASDEARKHTY |
| | | | NLTIAWYRMGDNCAIPITVMEYTECPYNKSLGVCPI |
| | | | RTQPRWSYYDSFSAVSEDNLGFLMHAPAFETAGTY |
| | | | LRLVKINDWTEITQFILEHRARASCKYALPLRIPPAA |
| | | | CLTSKAYQQGVTVDSIGMLPRFIPENQRTVALYSLKI |
| | | | AGWHGPKPPYTSTLLPPELSDTTNATQPELVPEDPED |
| | | | SALLEDPAGTVSSQIPPNWHIPSIQDVAPHH |
| 70 | HSV-2 gD | HSV-2 gD | MGRLTSGVGTAALLVVAVGLRVVCAKYALADPS |
| | | (26-331) | LKMADPNRFRGKNLPVLDQLTDPPGVKRVYHIQPSL |
| | | | EDPFQPPSIPITVYYAVLERACRSVLLHAPSEAPQIVR |
| | | | GASDEARKHTYNLTIAWYRMGDNCAIPITVMEYTE |
| | | | CPYNKSLGVCPIRTQPRWSYYDSFSAVSEDNLGFLM |
| | | | HAPAFETAGTYLRLVKINDWTEITQFILEHRARASCK |
| | | | YALPLRIPPAACLTSKAYQQGVTVDSIGMLPRFIPEN |
| | | | QRTVALYSLKIAGWHGPKPPYTSTLLPPELSDTTNAT |
| | | | QPELVPEDPEDSALLEDPAGTVSSQIPPNWHIPSIQDV |
| | | | APHH |
| 71 | HSV-2 | HSV-2 gD | MGRLTSGVGTAALLVVAVGLRVVCAKYADPSLK |
| | gD − KY | (30-331) | MADPNRFRGKNLPVLDQLTDPPGVKRVYHIQPSLED |
| | | | PFQPPSIPITVYYAVLERACRSVLLHAPSEAPQIVRGA |
| | | | SDEARKHTYNLTIAWYRMGDNCAIPITVMEYTECPY |
| | | | NKSLGVCPIRTQPRWSYYDSFSAVSEDNLGFLMHAP |
| | | | AFETAGTYLRLVKINDWTEITQFILEHRARASCKYAL |
| | | | PLRIPPAACLTSKAYQQGVTVDSIGMLPRFIPENQRT |
| | | | VALYSLKIAGWHGPKPPYTSTLLPPELSDTTNATQPE |
| | | | LVPEDPEDSALLEDPAGTVSSQIPPNWHIPSIQDVAP |
| | | | HH |
| 73 | IL2 | HSV-2 gE | MRMQLLLLIALSLALVTNSRTSWKRVTSGEDVVL |
| | | (24-405) | LPAPAGPEERTRAHKLLWAAEPLDACGPLRPSWVA |
| | | | LWPPRRVLETVVDAACMRAPEPLAIAYSPPFPAGDE |
| | | | GLYSELAWRDRVAVVNESLVIYGALETDSGLYTLSV |
| | | | VGLSDEARQVASVVLVVEPAPVPTPTPDDYDEEDD |
| | | | AGVSERTPVSVPPPTPPRRPPVAPPTHPRVIPEVSHVR |
| | | | GVTVHMETPEAILFAPGETFGTNVSIHAIAHDDGPYA |
| | | | MDVVWMRFDVPSSCAEMRIYEACLYHPQLPECLSP |
| | | | ADAPCAVSSWAYRLAVRSYAGCSRTTPPPRCFAEAR |
| | | | MEPVPGLAWLASTVNLEFQHASPQHAGLYLCVVYV |
| | | | DDHIHAWGHMTISTAAQYRNAVVEQHLPQRQPEPV |
| | | | EPTRPHVRA |
| 74 | HSV-2 gD | HSV-2 gE | MGRLTSGVGTAALLVVAVGLRVVCARTSWKRVT |
| | | (24-405) | SGEDVVLLPAPAGPEERTRAHKLLWAAEPLDACGPL |
| | | | RPSWVALWPPRRVLETVVDAACMRAPEPLAIAYSPP |
| | | | FPAGDEGLYSELAWRDRVAVVNESLVIYGALETDS |
| | | | GLYTLSVVGLSDEARQVASVVLVVEPAPVPTPTPDD |
| | | | YDEEDDAGVSERTPVSVPPPTPPRRPPVAPPTHPRVIP |
| | | | EVSHVRGVTVHMETPEAILFAPGETFGTNVSIHAIAH |
| | | | DDGPYAMDVVWMRFDVPSSCAEMRIYEACLYHPQ |
| | | | LPECLSPADAPCAVSSWAYRLAVRSYAGCSRTTPPP |
| | | | RCFAEARMEPVPGLAWLASTVNLEFQHASPQHAGL |
| | | | YLCVVYVDDHIHAWGHMTISTAAQYRNAVVEQHL |
| | | | PQRQPEPVEPTRPHVRA |
| 75 | HSV-2 | HSV-2 gE | MGRLTSGVGTAALLVVAVGLRVVCAKYARTSWK |
| | gD − KYA | (24-405) | RVTSGEDVVLLPAPAGPEERTRAHKLLWAAEPLDA |
| | | | CGPLRPSWVALWPPRRVLETVVDAACMRAPEPLAI |
| | | | AYSPPFPAGDEGLYSELAWRDRVAVVNESLVIYGAL |
| | | | ETDSGLYTLSVVGLSDEARQVASVVLVVEPAPVPTP |
| | | | TPDDYDEEDDAGVSERTPVSVPPPTPPRRPPVAPPTH |
| | | | PRVIPEVSHVRGVTVHMETPEAILFAPGETFGTNVSI |
| | | | HAIAHDDGPYAMDVVWMRFDVPSSCAEMRIYEACL |
| | | | YHPQLPECLSPADAPCAVSSWAYRLAVRSYAGCSRT |
| | | | TPPPRCFAEARMEPVPGLAWLASTVNLEFQHASPQH |
| | | | AGLYLCVVYVDDHIHAWGHMTISTAAQYRNAVVE |
| | | | QHLPQRQPEPVEPTRPHVRA |
| 76 | HSV-2 | HSV-2 gE | MGRLTSGVGTAALLVVAVGLRVVCAKYALARTS |
| | gD − KYALA | (24-405) | WKRVTSGEDVVLLPAPAGPEERTRAHKLLWAAEPL |
| | | | DACGPLRPSWVALWPPRRVLETVVDAACMRAPEPL |
| | | | AIAYSPPFPAGDEGLYSELAWRDRVAVVNESLVIYG |
| | | | ALETDSGLYTLSVVGLSDEARQVASVVLVVEPAPVP |
| | | | TPTPDDYDEEDDAGVSERTPVSVPPPTPPRRPPVAPP |
| | | | THPRVIPEVSHVRGVTVHMETPEAILFAPGETFGTNV |
| | | | SIHAIAHDDGPYAMDVVWMRFDVPSSCAEMRIYEA |
| | | | CLYHPQLPECLSPADAPCAVSSWAYRLAVRSYAGCS |
| | | | RTTPPPRCFAEARMEPVPGLAWLASTVNLEFQHASP |
| | | | QHAGLYLCVVYVDDHIHAWGHMTISTAAQYRNAV |
| | | | VEQHLPQRQPEPVEPTRPHVRA |
| 132 | HSV-2 gE | HSV-2 gE | MARGAGLVFFVGVWVVSCLAAAPRTSWKRVTSG |
| | | (24-405) | EDVVLLPAPAGPEERTRAHKLLWAAEPLDACGPLRP |
| | | | SWVALWPPRRVLETVVDAACMRAPEPLAIAYSPPFP |
| | | | AGDEGLYSELAWRDRVAVVNESLVIYGALETDSGL |
| | | | YTLSVVGLSDEARQVASVVLVVEPAPVPTPTPDDYD |
| | | | EEDDAGVSERTPVSVPPPTPPRRPPVAPPTHPRVIPEV |
| | | | SHVRGVTVHMETPEAILFAPGETFGTNVSIHAIAHDD |
| | | | GPYAMDVVWMRFDVPSSCAEMRIYEACLYHPQLPE |
| | | | CLSPADAPCAVSSWAYRLAVRSYAGCSRTTPPPRCF |
| | | | AEARMEPVPGLAWLASTVNLEFQHASPQHAGLYLC |
| | | | VVYVDDHIHAWGHMTISTAAQYRNAVVEQHLPQR |
| | | | QPEPVEPTRPHVRA |
| 327 | HSV-2 | HSV-2 gE | MARGAGLVFFVGVWVVSCLVVVPRTSWKRVTSG |
| | gE − LVVVP | (24-405) | EDVVLLPAPAGPEERTRAHKLLWAAEPLDACGPLRP |
| | | | SWVALWPPRRVLETVVDAACMRAPEPLAIAYSPPFP |
| | | | AGDEGLYSELAWRDRVAVVNESLVIYGALETDSGL |
| | | | YTLSVVGLSDEARQVASVVLVVEPAPVPTPTPDDYD |
| | | | EEDDAGVSERTPVSVPPPTPPRRPPVAPPTHPRVIPEV |
| | | | SHVRGVTVHMETPEAILFAPGETFGTNVSIHAIAHDD |
| | | | GPYAMDVVWMRFDVPSSCAEMRIYEACLYHPQLPE |
| | | | CLSPADAPCAVSSWAYRLAVRSYAGCSRTTPPPRCF |
| | | | AEARMEPVPGLAWLASTVNLEFQHASPQHAGLYLC |
| | | | VVYVDDHIHAWGHMTISTAAQYRNAVVEQHLPQR |
| | | | QPEPVEPTRPHVRA |
| 328 | None | HSV-2 gE | MRTSWKRVTSGEDVVLLPAPAGPEERTRAHKLLWA |
| | (N-term | (24-405) | AEPLDACGPLRPSWVALWPPRRVLETVVDAACMRA |
| | Met) | | PEPLAIAYSPPFPAGDEGLYSELAWRDRVAVVNESL |
| | | | VIYGALETDSGLYTLSVVGLSDEARQVASVVLVVEP |
| | | | APVPTPTPDDYDEEDDAGVSERTPVSVPPPTPPRRPP |
| | | | VAPPTHPRVIPEVSHVRGVTVHMETPEAILFAPGETF |
| | | | GTNVSIHAIAHDDGPYAMDVVWMRFDVPSSCAEMR |
| | | | IYEACLYHPQLPECLSPADAPCAVSSWAYRLAVRSY |
| | | | AGCSRTTPPPRCFAEARMEPVPGLAWLASTVNLEFQ |
| | | | HASPQHAGLYLCVVYVDDHIHAWGHMTISTAAQYR |
| | | | NAVVEQHLPQRQPEPVEPTRPHVRA |
| 164 | HSV-1 | HSV-2 gE | MGGAAARLGAVILFVVIVGLHGVRGKYRTSWKR |
| | gD + KY | | VTSGEDVVLLPAPAGPEERTRAHKLLWAAEPLDAC |
| | | | GPLRPSWVALWPPRRVLETVVDAACMRAPEPLAIA |
| | | | YSPPFPAGDEGLYSELAWRDRVAVVNESLVIYGALE |
| | | | TDSGLYTLSVVGLSDEARQVASVVLVVEPAPVPTPT |
| | | | PDDYDEEDDAGVSERTPVSVPPPTPPRRPPVAPPTHP |
| | | | RVIPEVSHVRGVTVHMETPEAILFAPGETFGTNVSIH |
| | | | AIAHDDGPYAMDVVWMRFDVPSSCAEMRIYEACLY |
| | | | HPQLPECLSPADAPCAVSSWAYRLAVRSYAGCSRTT |
| | | | PPPRCFAEARMEPVPGLAWLASTVNLEFQHASPQHA |
| | | | GLYLCVVYVDDHIHAWGHMTISTAAQYRNAVVEQ |
| | | | HLPQRQPEPVEPTRPHVRA |

TABLE 12
Example Deoxyribonucleic Acid Sequences encoding secretory signals and HSV-
2 glycoproteins

| SEQ | | | | |
|---|---|---|---|---|
| ID | Secretory | Antigen/ | | |
| NO: | signal | OFR | Version | Sequences (Polynucleotides) |
| 77 | IL2 | HSV-2 | | ATGCGCATGCAGCTGCTGCTGCTGATCGCCCT |
| | | gC | | GTCCCTGGCCCTGGTGACCAACTCCGCCTCCC |
| | | | | CCGGCCGCACCATCACCGTGGGCCCCCGCGGC |
| | | | | AACGCCTCCAACGCCGCCCCCTCCGCCTCCCC |
| | | | | CCGCAACGCCTCCGCCCCCCGCACCACCCCCA |
| | | | | CCCCCCCCCAGCCCCGCAAGGCCACCAAGTCC |
| | | | | AAGGCCTCCACCGCCAAGCCCGCCCCCCCCCC |
| | | | | CAAGACCGGCCCCCCCAAGACCTCCTCCGAGC |
| | | | | CCGTGCGCTGCAACCGCCACGACCCCCTGGCC |
| | | | | CGCTACGGCTCCCGCGTGCAGATCCGCTGCCG |
| | | | | CTTCCCCAACTCCACCCGCACCGAGTTCCGCC |
| | | | | TGCAGATCTGGCGCTACGCCACCGCCACCGAC |
| | | | | GCCGAGATCGGCACCGCCCCCTCCCTGGAGG |
| | | | | AGGTGATGGTGAACGTGTCCGCCCCCCCCGGC |
| | | | | GGCCAGCTGGTGTACGACTCCGCCCCCAACCG |
| | | | | CACCGACCCCCACGTGATCTGGGCCGAGGGC |
| | | | | GCCGGCCCCGGCGCCTCCCCCCGCCTGTACTC |
| | | | | CGTGGTGGGCCCCCTGGGCCGCCAGCGCCTGA |
| | | | | TCATCGAGGAGCTGACCCTGGAGACCCAGGG |
| | | | | CATGTACTACTGGGTGTGGGGCCGCACCGACC |
| | | | | GCCCCTCCGCCTACGGCACCTGGGTGCGCGTG |
| | | | | CGCGTGTTCCGCCCCCCCTCCCTGACCATCCA |
| | | | | CCCCCACGCCGTGCTGGAGGGCCAGCCCTTCA |
| | | | | AGGCCACCTGCACCGCCGCCACCTACTACCCC |
| | | | | GGCAACCGCGCCGAGTTCGTGTGGTTCGAGG |
| | | | | ACGGCCGCCGCGTGTTCGACCCCGCCCAGATC |
| | | | | CACACCCAGACCCAGGAGAACCCCGACGGCT |
| | | | | TCTCCACCGTGTCCACCGTGACCTCCGCCGCC |
| | | | | GTGGGCGGCCAGGGCCCCCCCCGCACCTTCAC |
| | | | | CTGCCAGCTGACCTGGCACCGCGACTCCGTGT |
| | | | | CCTTCTCCCGCCGCAACGCCTCCGGCACCGCC |
| | | | | TCCGTGCTGCCCCGCCCCACCATCACCATGGA |
| | | | | GTTCACCGGCGACCACGCCGTGTGCACCGCCG |
| | | | | GCTGCGTGCCCGAGGGCGTGACCTTCGCCTGG |
| | | | | TTCCTGGGCGACGACTCCTCCCCCGCCGAGAA |
| | | | | GGTGGCCGTGGCCTCCCAGACCTCCTGCGGCC |
| | | | | GCCCCGGCACCGCCACCATCCGCTCCACCCTG |
| | | | | CCCGTGTCCTACGAGCAGACCGAGTACATCTG |
| | | | | CCGCCTGGCCGGCTACCCCGACGGCATCCCCG |
| | | | | TGCTGGAGCACCACTAA |
| 78 | IL2 | HSV-2 | Version | ATGAGAATGCAGCTGCTGCTCCTGATCGCCCT |
| | | gC | 1 | GTCTCTGGCCCTGGTCACCAATAGCGCTTCTC |
| | | | | CCGGCAGAACCATCACAGTGGGCCCTAGAGG |
| | | | | CAACGCCTCTAATGCCGCTCCTAGCGCCTCTC |
| | | | | CTAGAAACGCCTCTGCTCCCAGAACCACACCT |
| | | | | ACACCTCCACAGCCTAGAAAGGCCACCAAGA |
| | | | | GCAAGGCCAGCACAGCCAAACCTGCTCCTCC |
| | | | | ACCTAAGACAGGCCCTCCAAAGACAAGCTCT |
| | | | | GAGCCCGTGCGGTGCAACAGACACGATCCAC |
| | | | | TGGCCAGATACGGCAGCCGGGTGCAGATCAG |
| | | | | ATGCAGATTCCCCAACAGCACCCGGACCGAG |
| | | | | TTCCGGCTCCAGATTTGGAGATACGCCACCGC |
| | | | | CACAGATGCCGAGATTGGAACAGCCCCTAGC |
| | | | | CTGGAAGAAGTGATGGTCAACGTTTCAGCCCC |
| | | | | TCCTGGCGGCCAGCTGGTGTATGATTCTGCCC |
| | | | | CTAACCGGACCGATCCTCACGTGATATGGGCT |
| | | | | GAAGGTGCTGGCCCAGGCGCAAGCCCTAGAC |
| | | | | TGTATTCTGTTGTGGGCCCTCTGGGCAGACAG |
| | | | | CGGCTGATCATTGAGGAACTGACCCTGGAAA |
| | | | | CCCAGGGCATGTACTACTGGGTCTGGGGCAG |
| | | | | AACCGATAGACCAAGCGCCTATGGCACCTGG |
| | | | | GTTCGAGTGCGAGTGTTCAGACCTCCTAGCCT |
| | | | | GACCATCCATCCTCACGCCGTTCTGGAAGGCC |
| | | | | AGCCTTTCAAGGCCACATGTACCGCCGCCACC |
| | | | | TACTATCCCGGAAACAGAGCCGAGTTCGTTTG |
| | | | | GTTCGAGGACGGCAGAAGGGTGTTCGACCCC |
| | | | | GCTCAGATCCACACACAGACCCAAGAGAACC |
| | | | | CCGACGGCTTTAGCACCGTGTCCACAGTGACA |
| | | | | TCTGCCGCCGTTGGAGGACAGGGCCCTCCTAG |
| | | | | AACCTTTACCTGCCAGCTGACCTGGCACAGAG |
| | | | | ACAGCGTGTCCTTCAGCAGAAGAAACGCCAG |
| | | | | CGGCACAGCCAGCGTTCTGCCTAGACCTACCA |
| | | | | TCACCATGGAATTCACCGGCGACCACGCCGTG |
| | | | | TGTACAGCTGGATGTGTTCCTGAGGGCGTGAC |
| | | | | CTTCGCTTGGTTTCTGGGCGACGATAGCAGCC |
| | | | | CTGCCGAAAAAGTGGCTGTGGCCAGCCAGAC |
| | | | | AAGCTGTGGCAGACCTGGAACCGCCACCATC |
| | | | | AGAAGCACACTGCCTGTCAGCTACGAGCAGA |
| | | | | CCGAGTACATCTGTCGGCTGGCCGGCTATCCT |
| | | | | GATGGCATCCCTGTGCTGGAACACCACTGA |
| 79 | IL2 | HSV-2 | Version | ATGCGCATGCAACTGCTCCTGCTGATTGCGTT |
| | | gC | 2 | GAGCCTTGCCCTGGTGACCAACAGCGCAAGC |
| | | | | CCCGGCAGAACCATAACAGTAGGGCCACGGG |
| | | | | GGAATGCTTCCAATGCTGCACCTTCCGCTTCA |
| | | | | CCGAGGAATGCTTCTGCCCCAAGAACTACCCC |
| | | | | CACTCCTCCTCAACCCAGGAAAGCGACAAAG |
| | | | | TCCAAGGCCAGCACCGCAAAACCCGCTCCTCC |
| | | | | TCCAAAGACTGGGCCCCCAAAGACAAGTAGC |
| | | | | GAACCAGTTCGGTGCAACAGGCATGACCCAC |
| | | | | TTGCACGCTATGGGTCAAGAGTCCAGATACGG |
| | | | | TGTCGCTTCCCTAACAGTACAAGGACTGAGTT |
| | | | | TCGGCTGCAGATCTGGCGTTATGCCACAGCTA |
| | | | | CTGACGCAGAGATTGGTACCGCCCCCAGTTTG |
| | | | | GAAGAGGTGATGGTCAACGTGTCCGCACCCC |
| | | | | CAGGAGGACAGCTGGTCTATGACTCAGCGCC |
| | | | | CAATAGGACCGATCCCCACGTGATCTGGGCA |
| | | | | GAAGGAGCCGGTCCTGGGGCCTCTCCACGGCT |
| | | | | GTACTCAGTTGTTGGCCCGCTTGGACGACAGA |
| | | | | GACTCATCATCGAAGAGCTGACACTGGAGAC |
| | | | | ACAGGGGATGTACTACTGGGTGTGGGGCCGT |
| | | | | ACTGACCGCCCTTCCGCATATGGCACTTGGGT |
| | | | | GAGAGTTCGCGTCTTTCGGCCCCCTTCTCTCA |
| | | | | CCATCCATCCTCATGCCGTGCTCGAAGGCCAG |
| | | | | CCCTTTAAGGCCACATGCACTGCTGCGACCTA |
| | | | | CTACCCTGGCAACAGAGCCGAGTTTGTCTGGT |
| | | | | TTGAGGATGGTCGGCGAGTATTCGATCCAGCC |
| | | | | CAGATTCACACACAAACGCAGGAAAATCCGG |
| | | | | ACGGCTTCAGCACAGTGTCCACGGTGACCTCT |
| | | | | GCTGCAGTTGGTGGACAAGGACCCCCTCGAA |
| | | | | CCTTCACCTGTCAGCTGACCTGGCACAGAGAC |
| | | | | TCCGTAAGCTTCAGCCGTAGAAACGCCTCTGG |
| | | | | AACCGCCAGTGTGTTGCCGAGGCCGACTATCA |
| | | | | CGATGGAATTCACAGGCGATCATGCCGTCTGT |
| | | | | ACTGCCGGCTGTGTGCCAGAAGGCGTAACCTT |
| | | | | CGCTTGGTTTCTCGGGGATGACTCAAGTCCTG |
| | | | | CAGAGAAAGTGGCTGTGGCCTCTCAGACGAG |
| | | | | CTGCGGTCGACCAGGAACAGCTACCATTCGCA |
| | | | | GCACTCTGCCCGTGTCCTACGAGCAGACGGAG |
| | | | | TACATCTGCAGGCTGGCCGGCTATCCCGATGG |
| | | | | GATTCCAGTCCTGGAGCACCACTGA |
| 80 | IL2 | HSV-2 | Version | ATGAGAATGCAGCTGCTGCTGCTGATCGCCCT |
| | | gC | 3 | GTCCCTGGCCCTGGTGACCAACTCCGCCTCCC |
| | | | | CCGGCAGAACCATCACCGTGGGCCCCAGAGG |
| | | | | CAACGCCTCCAACGCCGCCCCCTCCGCCTCCC |
| | | | | CCAGAAACGCCTCCGCCCCCAGAACCACCCCC |
| | | | | ACCCCTCCCCAGCCCAGAAAAGCCACCAAAT |
| | | | | CCAAAGCCTCCACCGCCAAACCCGCCCCTCCT |
| | | | | CCCAAAACCGGCCCTCCCAAAACCTCCTCCGA |
| | | | | ACCCGTGAGATGCAACAGACACGATCCCCTG |
| | | | | GCCAGATACGGCTCCAGAGTGCAGATCAGAT |
| | | | | GCAGATTCCCCAACTCCACCAGAACCGAATTC |
| | | | | AGACTGCAGATCTGGAGATACGCCACCGCCA |
| | | | | CCGATGCCGAAATCGGCACCGCCCCCTCCCTG |
| | | | | GAAGAAGTGATGGTGAACGTGTCCGCCCCTCC |
| | | | | TGGCGGCCAGCTGGTGTACGATTCCGCCCCCA |
| | | | | ACAGAACCGATCCCCACGTGATCTGGGCCGA |
| | | | | AGGCGCCGGCCCCGGCGCCTCCCCCAGACTGT |
| | | | | ACTCCGTGGTGGGCCCCCTGGGCAGACAGAG |
| | | | | ACTGATCATCGAAGAACTGACCCTGGAAACC |
| | | | | CAGGGCATGTACTACTGGGTGTGGGGCAGAA |
| | | | | CCGATAGACCCTCCGCCTACGGCACCTGGGTG |
| | | | | AGAGTGAGAGTGTTCAGACCCCCTTCCCTGAC |
| | | | | CATCCACCCCCACGCCGTGCTGGAAGGCCAGC |
| | | | | CCTTCAAAGCCACCTGCACCGCCGCCACCTAC |
| | | | | TACCCCGGCAACAGAGCCGAATTCGTGTGGTT |
| | | | | CGAAGATGGCAGAAGGGTGTTCGATCCCGCC |
| | | | | CAGATCCACACCCAGACCCAGGAAAACCCCG |
| | | | | ACGGCTTCTCCACCGTGTCCACCGTGACCTCC |
| | | | | GCCGCCGTGGGCGGCCAGGGCCCTCCCAGAA |
| | | | | CCTTCACCTGCCAGCTGACCTGGCACAGAGAC |
| | | | | TCCGTGTCCTTCTCCAGAAGAAACGCCTCCGG |
| | | | | CACCGCCTCCGTGCTGCCCAGACCCACCATCA |
| | | | | CCATGGAATTCACCGGCGATCACGCCGTGTGC |
| | | | | ACCGCCGGCTGCGTGCCCGAAGGCGTGACCTT |
| | | | | CGCCTGGTTCCTGGGCGATGATTCCTCCCCCG |
| | | | | CCGAAAAAGTGGCCGTGGCCTCCCAGACCTCC |
| | | | | TGCGGCAGACCCGGCACCGCCACCATCAGAT |
| | | | | CCACCCTGCCCGTGTCCTACGAACAGACCGAA |
| | | | | TACATCTGCAGACTGGCCGGCTACCCCGATGG |
| | | | | CATCCCCGTGCTGGAACACCACTGA |
| 81 | HSV-2 | HSV-2 | | ATGGGCCGCCTGACCTCCGGCGTGGGCACCGC |
| | gD - | gC | | CGCCCTGCTGGTGGTGGCCGTGGGCCTGCGCG |
| | KYA | | | TGGTGTGCGCCAAGTACGCCGCCTCCCCCGGC |
| | | | | CGCACCATCACCGTGGGCCCCCGCGGCAACG |
| | | | | CCTCCAACGCCGCCCCCTCCGCCTCCCCCCGC |
| | | | | AACGCCTCCGCCCCCCGCACCACCCCCACCCC |
| | | | | CCCCCAGCCCCGCAAGGCCACCAAGTCCAAG |
| | | | | GCCTCCACCGCCAAGCCCGCCCCCCCCCCCAA |
| | | | | GACCGGCCCCCCCAAGACCTCCTCCGAGCCCG |
| | | | | TGCGCTGCAACCGCCACGACCCCCTGGCCCGC |
| | | | | TACGGCTCCCGCGTGCAGATCCGCTGCCGCTT |
| | | | | CCCCAACTCCACCCGCACCGAGTTCCGCCTGC |
| | | | | AGATCTGGCGCTACGCCACCGCCACCGACGCC |
| | | | | GAGATCGGCACCGCCCCCTCCCTGGAGGAGG |
| | | | | TGATGGTGAACGTGTCCGCCCCCCCCGGCGGC |
| | | | | CAGCTGGTGTACGACTCCGCCCCCAACCGCAC |
| | | | | CGACCCCCACGTGATCTGGGCCGAGGGCGCC |
| | | | | GGCCCCGGCGCCTCCCCCCGCCTGTACTCCGT |
| | | | | GGTGGGCCCCCTGGGCCGCCAGCGCCTGATCA |
| | | | | TCGAGGAGCTGACCCTGGAGACCCAGGGCAT |
| | | | | GTACTACTGGGTGTGGGGCCGCACCGACCGCC |
| | | | | CCTCCGCCTACGGCACCTGGGTGCGCGTGCGC |
| | | | | GTGTTCCGCCCCCCCTCCCTGACCATCCACCC |
| | | | | CCACGCCGTGCTGGAGGGCCAGCCCTTCAAG |
| | | | | GCCACCTGCACCGCCGCCACCTACTACCCCGG |
| | | | | CAACCGCGCCGAGTTCGTGTGGTTCGAGGACG |
| | | | | GCCGCCGCGTGTTCGACCCCGCCCAGATCCAC |
| | | | | ACCCAGACCCAGGAGAACCCCGACGGCTTCT |
| | | | | CCACCGTGTCCACCGTGACCTCCGCCGCCGTG |
| | | | | GGCGGCCAGGGCCCCCCCCGCACCTTCACCTG |
| | | | | CCAGCTGACCTGGCACCGCGACTCCGTGTCCT |
| | | | | TCTCCCGCCGCAACGCCTCCGGCACCGCCTCC |
| | | | | GTGCTGCCCCGCCCCACCATCACCATGGAGTT |
| | | | | CACCGGCGACCACGCCGTGTGCACCGCCGGCT |
| | | | | GCGTGCCCGAGGGCGTGACCTTCGCCTGGTTC |
| | | | | CTGGGCGACGACTCCTCCCCCGCCGAGAAGGT |
| | | | | GGCCGTGGCCTCCCAGACCTCCTGCGGCCGCC |
| | | | | CCGGCACCGCCACCATCCGCTCCACCCTGCCC |
| | | | | GTGTCCTACGAGCAGACCGAGTACATCTGCCG |
| | | | | CCTGGCCGGCTACCCCGACGGCATCCCCGTGC |
| | | | | TGGAGCACCACTAA |
| 82 | HSV-2 | HSV-2 | Version | ATGGGGAGACTCACATCAGGCGTAGGAACCG |
| | gD - | gC | 2 | CTGCCCTGTTGGTCGTGGCCGTTGGTCTGAGA |
| | KYA | | | GTTGTGTGTGCCAAATATGCTGCAAGCCCCGG |
| | | | | CAGAACCATAACAGTAGGGCCACGGGGGAAT |
| | | | | GCTTCCAATGCTGCACCTTCCGCTTCACCGAG |
| | | | | GAATGCTTCTGCCCCAAGAACTACCCCCACTC |
| | | | | CTCCTCAACCCAGGAAAGCGACAAAGTCCAA |
| | | | | GGCCAGCACCGCAAAACCCGCTCCTCCTCCAA |
| | | | | AGACTGGGCCCCCAAAGACAAGTAGCGAACC |
| | | | | AGTTCGGTGCAACAGGCATGACCCACTTGCAC |
| | | | | GCTATGGGTCAAGAGTCCAGATACGGTGTCGC |
| | | | | TTCCCTAACAGTACAAGGACTGAGTTTCGGCT |
| | | | | GCAGATCTGGCGTTATGCCACAGCTACTGACG |
| | | | | CAGAGATTGGTACCGCCCCCAGTTTGGAAGA |
| | | | | GGTGATGGTCAACGTGTCCGCACCCCCAGGA |
| | | | | GGACAGCTGGTCTATGACTCAGCGCCCAATAG |
| | | | | GACCGATCCCCACGTGATCTGGGCAGAAGGA |
| | | | | GCCGGTCCTGGGGCCTCTCCACGGCTGTACTC |
| | | | | AGTTGTTGGCCCGCTTGGACGACAGAGACTCA |
| | | | | TCATCGAAGAGCTGACACTGGAGACACAGGG |
| | | | | GATGTACTACTGGGTGTGGGGCCGTACTGACC |
| | | | | GCCCTTCCGCATATGGCACTTGGGTGAGAGTT |
| | | | | CGCGTCTTTCGGCCCCCTTCTCTCACCATCCAT |
| | | | | CCTCATGCCGTGCTCGAAGGCCAGCCCTTTAA |
| | | | | GGCCACATGCACTGCTGCGACCTACTACCCTG |
| | | | | GCAACAGAGCCGAGTTTGTCTGGTTTGAGGAT |
| | | | | GGTCGGCGAGTATTCGATCCAGCCCAGATTCA |
| | | | | CACACAAACGCAGGAAAATCCGGACGGCTTC |
| | | | | AGCACAGTGTCCACGGTGACCTCTGCTGCAGT |
| | | | | TGGTGGACAAGGACCCCCTCGAACCTTCACCT |
| | | | | GTCAGCTGACCTGGCACAGAGACTCCGTAAG |
| | | | | CTTCAGCCGTAGAAACGCCTCTGGAACCGCCA |
| | | | | GTGTGTTGCCGAGGCCGACTATCACGATGGAA |
| | | | | TTCACAGGCGATCATGCCGTCTGTACTGCCGG |
| | | | | CTGTGTGCCAGAAGGCGTAACCTTCGCTTGGT |
| | | | | TTCTCGGGGATGACTCAAGTCCTGCAGAGAAA |
| | | | | GTGGCTGTGGCCTCTCAGACGAGCTGCGGTCG |
| | | | | ACCAGGAACAGCTACCATTCGCAGCACTCTGC |
| | | | | CCGTGTCCTACGAGCAGACGGAGTACATCTGC |
| | | | | AGGCTGGCCGGCTATCCCGATGGGATTCCAGT |
| | | | | CCTGGAGCACCACTGA |
| 83 | HSV-2 | HSV-2 | Version | ATGGGGAGACTCACATCAGGCGTAGGAACCG |
| | gD - | gC | 2.1 | CTGCCCTGTTGGTCGTGGCCGTTGGTCTGAGA |
| | KYA | | | GTTGTGTGTGCCAAATATGCTGCAAGCCCCGG |
| | | | | CAGAACCATAACAGTAGGGCCACGGGGGAAT |
| | | | | GCTTCCAATGCTGCACCTTCCGCTTCACCGAG |
| | | | | GAATGCTTCTGCCCCAAGAACTACCCCCACTC |
| | | | | CTCCTCAACCCAGGAAAGCGACAAAGTCCAA |
| | | | | GGCCAGCACCGCAAAACCCGCTCCTCCTCCAA |
| | | | | AGACTGGGCCCCCAAAGACAAGTAGCGAACC |
| | | | | AGTTCGGTGCAACAGGCATGACCCACTTGCAC |
| | | | | GCTATGGGTCAAGAGTCCAGATACGGTGTCGC |
| | | | | TTCCCTAACAGTACAAGGACTGAGTTTCGGCT |
| | | | | GCAGATCTGGCGTTATGCCACAGCTACTGACG |
| | | | | CAGAGATTGGTACCGCCCCCAGTTTGGAGGA |
| | | | | AGTGATGGTCAACGTGTCCGCACCCCCAGGA |
| | | | | GGACAGCTGGTCTATGACTCAGCGCCCAATAG |
| | | | | GACCGATCCCCACGTGATCTGGGCAGAAGGA |
| | | | | GCCGGTCCTGGGGCCTCTCCACGGCTGTACTC |
| | | | | AGTTGTTGGCCCGCTTGGACGACAGAGACTCA |
| | | | | TCATCGAGGAACTGACACTGGAGACACAGGG |
| | | | | GATGTACTACTGGGTGTGGGGCCGTACTGACC |
| | | | | GCCCTTCCGCATATGGCACTTGGGTGAGAGTT |
| | | | | CGCGTCTTTCGGCCCCCTTCTCTCACCATCCAT |
| | | | | CCTCATGCCGTGCTCGAAGGCCAGCCCTTTAA |
| | | | | GGCCACATGCACTGCTGCGACCTACTACCCTG |
| | | | | GCAACAGAGCCGAGTTTGTCTGGTTTGAGGAT |
| | | | | GGTCGGCGAGTATTCGATCCAGCCCAGATTCA |
| | | | | CACACAAACGCAGGAAAATCCGGACGGCTTC |
| | | | | AGCACAGTGTCCACGGTGACCTCTGCTGCAGT |
| | | | | TGGTGGACAAGGACCCCCTCGAACCTTCACCT |
| | | | | GTCAGCTGACCTGGCACAGAGACTCCGTAAG |
| | | | | CTTCAGCCGTAGAAACGCCTCTGGAACCGCCA |
| | | | | GTGTGTTGCCGAGGCCGACTATCACGATGGAA |
| | | | | TTCACAGGCGATCATGCCGTCTGTACTGCCGG |
| | | | | CTGTGTGCCAGAAGGCGTAACCTTCGCTTGGT |
| | | | | TTCTCGGGGATGACTCAAGTCCTGCAGAGAAA |
| | | | | GTGGCTGTGGCCTCTCAGACGAGCTGCGGTCG |
| | | | | ACCAGGAACAGCTACCATTCGCAGCACTCTGC |
| | | | | CCGTGTCCTACGAGCAGACGGAGTACATCTGC |
| | | | | AGGCTGGCCGGCTATCCCGATGGGATTCCAGT |
| | | | | CCTGGAGCACCACTGA |
| 84 | HSV-2 | HSV-2 | Version | ATGGGGAGACTCACATCAGGCGTAGGAACCG |
| | gD - | gC | 2.2 | CTGCCCTGTTGGTCGTGGCCGTTGGTCTGAGA |
| | KYA | | | GTTGTGTGTGCCAAATATGCTGCAAGCCCCGG |
| | | | | CAGAACCATAACAGTAGGGCCACGGGGGAAT |
| | | | | GCTTCCAATGCTGCACCTTCCGCTTCACCGAG |
| | | | | GAATGCTTCTGCCCCAAGAACTACCCCCACTC |
| | | | | CTCCTCAACCCAGGAAAGCGACAAAGTCCAA |
| | | | | GGCCAGCACCGCAAAACCCGCTCCTCCTCCAA |
| | | | | AGACTGGGCCCCCAAAGACAAGTAGCGAACC |
| | | | | AGTTCGGTGCAACAGGCATGACCCACTTGCAC |
| | | | | GCTATGGGTCAAGAGTCCAGATACGGTGTCGC |
| | | | | TTCCCTAACAGTACAAGGACTGAGTTTCGGCT |
| | | | | GCAGATCTGGCGTTATGCCACAGCTACTGACG |
| | | | | CAGAGATTGGTACCGCCCCCAGTTTGGAAGA |
| | | | | GGTGATGGTCAACGTGTCCGCACCCCCAGGA |
| | | | | GGACAGCTGGTCTATGACTCAGCGCCCAATAG |
| | | | | GACCGATCCCCACGTGATCTGGGCAGAAGGA |
| | | | | GCCGGTCCTGGGGCCTCTCCACGGCTGTACTC |
| | | | | AGTTGTTGGCCCGCTTGGACGACAGAGACTCA |
| | | | | TCATCGAAGAGCTGACACTGGAGACACAGGG |
| | | | | GATGTACTACTGGGTGTGGGGCCGTACTGACC |
| | | | | GCCCTTCCGCATATGGCACTTGGGTGAGAGTT |
| | | | | CGCGTCTTTCGGCCCCCTTCTCTCACCATCCAT |
| | | | | CCTCATGCCGTGCTCGAAGGCCAGCCCTTTAA |
| | | | | GGCCACATGCACTGCTGCGACCTACTACCCTG |
| | | | | GCAACAGAGCCGAGTTTGTCTGGTTTGAGGAT |
| | | | | GGTCGGCGAGTATTCGATCCAGCCCAGATTCA |
| | | | | CACACAAACGCAGGAAAATCCGGACGGCTTC |
| | | | | AGCACAGTGTCCACGGTGACCTCTGCTGCAGT |
| | | | | TGGTGGACAAGGACCCCCTCGAACCTTCACCT |
| | | | | GTCAGCTGACCTGGCACAGAGACTCCGTAAG |
| | | | | CTTCAGCCGTAGAAACGCCTCTGGAACCGCCA |
| | | | | GTGTGTTGCCGAGGCCGACTATCACGATGGAA |
| | | | | TTCACAGGCGATCATGCCGTCTGTACTGCCGG |
| | | | | CTGTGTGCCAGAAGGCGTAACCTTCGCTTGGT |
| | | | | TTCTCGGGGATGACTCAAGTCCTGCAGAGAAA |
| | | | | GTGGCTGTGGCCTCTCAGACGAGCTGCGGTCG |
| | | | | ACCAGGAACAGCTACCATTCGCAGCACTCTGC |
| | | | | CCGTGTCCTACGAGCAGACGGAGTACATCTGC |
| | | | | AGGCTGGCCGGCTATCCCGATGGGATTCCAGT |
| | | | | CCTGGAGCACCACTGA |
| 85 | HSV-2 | HSV-2 | Version | ATGGGCAGACTGACATCTGGCGTGGGAACAG |
| | gD - | gC | 1 | CTGCTCTGCTGGTGGTTGCTGTGGGCCTGAGA |
| | KYALA | | | GTCGTGTGTGCCAAATACGCCCTGGCCTCTCC |
| | | | | CGGCAGAACCATCACAGTGGGCCCTAGAGGC |
| | | | | AACGCCTCTAATGCCGCTCCTAGCGCCTCTCC |
| | | | | TAGAAACGCCTCTGCTCCCAGAACCACACCTA |
| | | | | CACCTCCACAGCCTAGAAAGGCCACCAAGAG |
| | | | | CAAGGCCAGCACAGCCAAACCTGCTCCTCCAC |
| | | | | CTAAGACAGGCCCTCCAAAGACAAGCTCTGA |
| | | | | GCCCGTGCGGTGCAACAGACACGATCCACTG |
| | | | | GCCAGATACGGCAGCCGGGTGCAGATCAGAT |
| | | | | GCAGATTCCCCAACAGCACCCGGACCGAGTTC |
| | | | | CGGCTCCAGATTTGGAGATACGCCACCGCCAC |
| | | | | AGATGCCGAGATTGGAACAGCCCCTAGCCTG |
| | | | | GAAGAAGTGATGGTCAACGTTTCAGCCCCTCC |
| | | | | TGGCGGCCAGCTGGTGTATGATTCTGCCCCTA |
| | | | | ACCGGACCGATCCTCACGTGATATGGGCTGAA |
| | | | | GGTGCTGGCCCAGGCGCAAGCCCTAGACTGT |
| | | | | ATTCTGTTGTGGGCCCTCTGGGCAGACAGCGG |
| | | | | CTGATCATTGAGGAACTGACCCTGGAAACCCA |
| | | | | GGGCATGTACTACTGGGTCTGGGGCAGAACC |
| | | | | GATAGACCAAGCGCCTATGGCACCTGGGTTCG |
| | | | | AGTGCGAGTGTTCAGACCTCCTAGCCTGACCA |
| | | | | TCCATCCTCACGCCGTTCTGGAAGGCCAGCCT |
| | | | | TTCAAGGCCACATGTACCGCCGCCACCTACTA |
| | | | | TCCCGGAAACAGAGCCGAGTTCGTTTGGTTCG |
| | | | | AGGACGGCAGAAGGGTGTTCGACCCCGCTCA |
| | | | | GATCCACACACAGACCCAAGAGAACCCCGAC |
| | | | | GGCTTTAGCACCGTGTCCACAGTGACATCTGC |
| | | | | CGCCGTTGGAGGACAGGGCCCTCCTAGAACCT |
| | | | | TTACCTGCCAGCTGACCTGGCACAGAGACAGC |
| | | | | GTGTCCTTCAGCAGAAGAAACGCCAGCGGCA |
| | | | | CAGCCAGCGTTCTGCCTAGACCTACCATCACC |
| | | | | ATGGAATTCACCGGCGACCACGCCGTGTGTAC |
| | | | | AGCTGGATGTGTTCCTGAGGGCGTGACCTTCG |
| | | | | CTTGGTTTCTGGGCGACGATAGCAGCCCTGCC |
| | | | | GAAAAAGTGGCTGTGGCCAGCCAGACAAGCT |
| | | | | GTGGCAGACCTGGAACCGCCACCATCAGAAG |
| | | | | CACACTGCCTGTCAGCTACGAGCAGACCGAGT |
| | | | | ACATCTGTCGGCTGGCCGGCTATCCTGATGGC |
| | | | | ATCCCTGTGCTGGAACACCACTGA |
| 86 | HSV-2 | HSV-2 | Version | ATGGGGAGACTCACATCAGGCGTAGGAACCG |
| | gD - | gC | 2 | CTGCCCTGTTGGTCGTGGCCGTTGGTCTGAGA |
| | KYALA | | | GTTGTGTGTGCCAAATATGCTCTGGCAAGCCC |
| | | | | CGGCAGAACCATAACAGTAGGGCCACGGGGG |
| | | | | AATGCTTCCAATGCTGCACCTTCCGCTTCACC |
| | | | | GAGGAATGCTTCTGCCCCAAGAACTACCCCCA |
| | | | | CTCCTCCTCAACCCAGGAAAGCGACAAAGTCC |
| | | | | AAGGCCAGCACCGCAAAACCCGCTCCTCCTCC |
| | | | | AAAGACTGGGCCCCCAAAGACAAGTAGCGAA |
| | | | | CCAGTTCGGTGCAACAGGCATGACCCACTTGC |
| | | | | ACGCTATGGGTCAAGAGTCCAGATACGGTGTC |
| | | | | GCTTCCCTAACAGTACAAGGACTGAGTTTCGG |
| | | | | CTGCAGATCTGGCGTTATGCCACAGCTACTGA |
| | | | | CGCAGAGATTGGTACCGCCCCCAGTTTGGAGG |
| | | | | AAGTGATGGTCAACGTGTCCGCACCCCCAGG |
| | | | | AGGACAGCTGGTCTATGACTCAGCGCCCAATA |
| | | | | GGACCGATCCCCACGTGATCTGGGCAGAAGG |
| | | | | AGCCGGTCCTGGGGCCTCTCCACGGCTGTACT |
| | | | | CAGTTGTTGGCCCGCTTGGACGACAGAGACTC |
| | | | | ATCATCGAGGAACTGACACTGGAGACACAGG |
| | | | | GGATGTACTACTGGGTGTGGGGCCGTACTGAC |
| | | | | CGCCCTTCCGCATATGGCACTTGGGTGAGAGT |
| | | | | TCGCGTCTTTCGGCCCCCTTCTCTCACCATCCA |
| | | | | TCCTCATGCCGTGCTCGAAGGCCAGCCCTTTA |
| | | | | AGGCCACATGCACTGCTGCGACCTACTACCCT |
| | | | | GGCAACAGAGCCGAGTTTGTCTGGTTTGAGGA |
| | | | | TGGTCGGCGAGTATTCGATCCAGCCCAGATTC |
| | | | | ACACACAAACGCAGGAAAATCCGGACGGCTT |
| | | | | CAGCACAGTGTCCACGGTGACCTCTGCTGCAG |
| | | | | TTGGTGGACAAGGACCCCCTCGAACCTTCACC |
| | | | | TGTCAGCTGACCTGGCACAGAGACTCCGTAAG |
| | | | | CTTCAGCCGTAGAAACGCCTCTGGAACCGCCA |
| | | | | GTGTGTTGCCGAGGCCGACTATCACGATGGAA |
| | | | | TTCACAGGCGATCATGCCGTCTGTACTGCCGG |
| | | | | CTGTGTGCCAGAAGGCGTAACCTTCGCTTGGT |
| | | | | TTCTCGGGGATGACTCAAGTCCTGCAGAGAAA |
| | | | | GTGGCTGTGGCCTCTCAGACGAGCTGCGGTCG |
| | | | | ACCAGGAACAGCTACCATTCGCAGCACTCTGC |
| | | | | CCGTGTCCTACGAGCAGACGGAGTACATCTGC |
| | | | | AGGCTGGCCGGCTATCCCGATGGGATTCCAGT |
| | | | | CCTGGAGCACCACTGA |
| 309 | HSV-2 | HSV-2 | Version | ATGGGGAGACTCACATCCGGAGTGGGCACTG |
| | gD - | gC | 2.1 | CTGCCCTGTTGGTCGTGGCCGTTGGTCTGAGA |
| | KYALA | | | GTTGTGTGTGCCAAATATGCTCTGGCAAGCCC |
| | | | | CGGCAGAACCATAACAGTAGGGCCACGGGGG |
| | | | | AATGCTTCCAATGCTGCACCTTCCGCTTCACC |
| | | | | GAGGAATGCTTCTGCCCCAAGAACTACCCCCA |
| | | | | CTCCTCCTCAACCCAGGAAAGCGACAAAGTCC |
| | | | | AAGGCCAGCACCGCAAAACCCGCTCCTCCTCC |
| | | | | AAAGACTGGGCCCCCAAAGACAAGTAGCGAA |
| | | | | CCAGTTCGGTGCAACAGGCATGACCCACTTGC |
| | | | | ACGCTATGGGTCAAGAGTCCAGATACGGTGTC |
| | | | | GCTTCCCTAACAGTACAAGGACTGAGTTTCGG |
| | | | | CTGCAGATCTGGCGTTATGCCACAGCTACTGA |
| | | | | CGCAGAGATTGGTACCGCCCCCAGTTTGGAGG |
| | | | | AAGTGATGGTCAACGTGTCCGCACCCCCAGG |
| | | | | AGGACAGCTGGTCTATGACTCAGCGCCCAATA |
| | | | | GGACAGACCCACATGTTATCTGGGCAGAAGG |
| | | | | AGCCGGTCCTGGGGCCTCTCCACGGCTGTACT |
| | | | | CAGTTGTTGGCCCGCTTGGACGACAGAGACTC |
| | | | | ATCATCGAGGAACTGACACTGGAGACACAGG |
| | | | | GGATGTACTACTGGGTGTGGGGCCGTACTGAC |
| | | | | CGCCCTTCCGCATATGGCACTTGGGTGAGAGT |
| | | | | TCGCGTCTTTCGGCCCCCTTCTCTCACCATCCA |
| | | | | TCCTCATGCCGTGCTCGAAGGCCAGCCCTTTA |
| | | | | AGGCCACATGCACTGCTGCGACCTACTACCCT |
| | | | | GGCAACAGAGCCGAGTTTGTCTGGTTTGAGGA |
| | | | | TGGTCGGCGAGTATTCGATCCAGCCCAGATTC |
| | | | | ACACACAAACGCAGGAAAATCCGGACGGCTT |
| | | | | CAGCACAGTGTCCACGGTGACCTCTGCTGCAG |
| | | | | TTGGTGGACAAGGACCCCCTCGAACCTTCACC |
| | | | | TGTCAGCTGACCTGGCACAGAGACTCCGTAAG |
| | | | | CTTCAGCCGTAGAAACGCCTCTGGAACCGCCA |
| | | | | GTGTGTTGCCGAGGCCGACTATCACGATGGAA |
| | | | | TTCACAGGCGATCATGCCGTCTGTACTGCCGG |
| | | | | CTGTGTGCCAGAAGGCGTAACCTTCGCTTGGT |
| | | | | TTCTCGGGGATGACTCAAGTCCTGCAGAGAAA |
| | | | | GTGGCTGTGGCCTCTCAGACGAGCTGCGGTCG |
| | | | | ACCAGGAACAGCTACCATTCGCAGCACTCTGC |
| | | | | CCGTGTCCTACGAGCAGACGGAGTACATCTGC |
| | | | | AGGCTGGCCGGCTATCCCGATGGGATTCCAGT |
| | | | | CCTGGAGCACCACTGA |
| 87 | HSV-2 | HSV-2 | Version | ATGGGCAGACTGACCTCCGGCGTGGGCACCG |
| | gD - | gC | 3 | CCGCCCTGCTGGTGGTGGCCGTGGGCCTGAGA |
| | KYALA | | | GTGGTGTGCGCCAAATACGCCCTGGCCTCCCC |
| | | | | CGGCAGAACCATCACCGTGGGCCCCAGAGGC |
| | | | | AACGCCTCCAACGCCGCCCCCTCCGCCTCCCC |
| | | | | CAGAAACGCCTCCGCCCCCAGAACCACCCCC |
| | | | | ACCCCTCCCCAGCCCAGAAAAGCCACCAAAT |
| | | | | CCAAAGCCTCCACCGCCAAACCCGCCCCTCCT |
| | | | | CCCAAAACCGGCCCTCCCAAAACCTCCTCCGA |
| | | | | ACCCGTGAGATGCAACAGACACGATCCCCTG |
| | | | | GCCAGATACGGCTCCAGAGTGCAGATCAGAT |
| | | | | GCAGATTCCCCAACTCCACCAGAACCGAATTC |
| | | | | AGACTGCAGATCTGGAGATACGCCACCGCCA |
| | | | | CCGATGCCGAAATCGGCACCGCCCCCTCCCTG |
| | | | | GAAGAAGTGATGGTGAACGTGTCCGCCCCTCC |
| | | | | TGGCGGCCAGCTGGTGTACGATTCCGCCCCCA |
| | | | | ACAGAACCGATCCCCACGTGATCTGGGCCGA |
| | | | | AGGCGCCGGCCCCGGCGCCTCCCCCAGACTGT |
| | | | | ACTCCGTGGTGGGCCCCCTGGGCAGACAGAG |
| | | | | ACTGATCATCGAAGAACTGACCCTGGAAACC |
| | | | | CAGGGCATGTACTACTGGGTGTGGGGCAGAA |
| | | | | CCGATAGACCCTCCGCCTACGGCACCTGGGTG |
| | | | | AGAGTGAGAGTGTTCAGACCCCCTTCCCTGAC |
| | | | | CATCCACCCCCACGCCGTGCTGGAAGGCCAGC |
| | | | | CCTTCAAAGCCACCTGCACCGCCGCCACCTAC |
| | | | | TACCCCGGCAACAGAGCCGAATTCGTGTGGTT |
| | | | | CGAAGATGGCAGAAGGGTGTTCGATCCCGCC |
| | | | | CAGATCCACACCCAGACCCAGGAAAACCCCG |
| | | | | ACGGCTTCTCCACCGTGTCCACCGTGACCTCC |
| | | | | GCCGCCGTGGGCGGCCAGGGCCCTCCCAGAA |
| | | | | CCTTCACCTGCCAGCTGACCTGGCACAGAGAC |
| | | | | TCCGTGTCCTTCTCCAGAAGAAACGCCTCCGG |
| | | | | CACCGCCTCCGTGCTGCCCAGACCCACCATCA |
| | | | | CCATGGAATTCACCGGCGATCACGCCGTGTGC |
| | | | | ACCGCCGGCTGCGTGCCCGAAGGCGTGACCTT |
| | | | | CGCCTGGTTCCTGGGCGATGATTCCTCCCCCG |
| | | | | CCGAAAAAGTGGCCGTGGCCTCCCAGACCTCC |
| | | | | TGCGGCAGACCCGGCACCGCCACCATCAGAT |
| | | | | CCACCCTGCCCGTGTCCTACGAACAGACCGAA |
| | | | | TACATCTGCAGACTGGCCGGCTACCCCGATGG |
| | | | | CATCCCCGTGCTGGAACACCACTGA |
| 88 | HSV-2 | HSV-2 | Version | ATGGCCTTGGGGAGAGTGGGCCTTGCAGTGG |
| | gC | gC | 2 | GTCTGTGGGGACTGCTCTGGGTTGGCGTAGTC |
| | | | | GTCGTGCTGGCTAACGCAAGCCCCGGCAGAA |
| | | | | CCATAACAGTAGGGCCACGGGGGAATGCTTC |
| | | | | CAATGCTGCACCTTCCGCTTCACCGAGGAATG |
| | | | | CTTCTGCCCCAAGAACTACCCCCACTCCTCCT |
| | | | | CAACCCAGGAAAGCGACAAAGTCCAAGGCCA |
| | | | | GCACCGCAAAACCCGCTCCTCCTCCAAAGACT |
| | | | | GGGCCCCCAAAGACAAGTAGCGAACCAGTTC |
| | | | | GGTGCAACAGGCATGACCCACTTGCACGCTAT |
| | | | | GGGTCAAGAGTCCAGATACGGTGTCGCTTCCC |
| | | | | TAACAGTACAAGGACTGAGTTTCGGCTGCAG |
| | | | | ATCTGGCGTTATGCCACAGCTACTGACGCAGA |
| | | | | GATTGGTACCGCCCCCAGTTTGGAGGAAGTGA |
| | | | | TGGTCAACGTGTCCGCACCCCCAGGAGGACA |
| | | | | GCTGGTCTATGACTCAGCGCCCAATAGGACCG |
| | | | | ATCCCCACGTGATCTGGGCAGAAGGAGCCGG |
| | | | | TCCTGGGGCCTCTCCACGGCTGTACTCAGTTG |
| | | | | TTGGCCCGCTTGGACGACAGAGACTCATCATC |
| | | | | GAGGAACTGACACTGGAGACACAGGGGATGT |
| | | | | ACTACTGGGTGTGGGGCCGTACTGACCGCCCT |
| | | | | TCCGCATATGGCACTTGGGTGAGAGTTCGCGT |
| | | | | CTTTCGGCCCCCTTCTCTCACCATCCATCCTCA |
| | | | | TGCCGTGCTCGAAGGCCAGCCCTTTAAGGCCA |
| | | | | CATGCACTGCTGCGACCTACTACCCTGGCAAC |
| | | | | AGAGCCGAGTTTGTCTGGTTTGAGGATGGTCG |
| | | | | GCGAGTATTCGATCCAGCCCAGATTCACACAC |
| | | | | AAACGCAGGAAAATCCGGACGGCTTCAGCAC |
| | | | | AGTGTCCACGGTGACCTCTGCTGCAGTTGGTG |
| | | | | GACAAGGACCCCCTCGAACCTTCACCTGTCAG |
| | | | | CTGACCTGGCACAGAGACTCCGTAAGCTTCAG |
| | | | | CCGTAGAAACGCCTCTGGAACCGCCAGTGTGT |
| | | | | TGCCGAGGCCGACTATCACGATGGAATTCACA |
| | | | | GGCGATCATGCCGTCTGTACTGCCGGCTGTGT |
| | | | | GCCAGAAGGCGTAACCTTCGCTTGGTTTCTCG |
| | | | | GGGATGACTCAAGTCCTGCAGAGAAAGTGGC |
| | | | | TGTGGCCTCTCAGACGAGCTGCGGTCGACCAG |
| | | | | GAACAGCTACCATTCGCAGCACTCTGCCCGTG |
| | | | | TCCTACGAGCAGACGGAGTACATCTGCAGGCT |
| | | | | GGCCGGCTATCCCGATGGGATTCCAGTCCTGG |
| | | | | AGCACCACTGA |
| 310 | HSV-2 | HSV-2 | Version | ATGGCACTAGGGAGAGTGGGATTAGCTGTGG |
| | gC | gC | 2.1 | GTCTGTGGGGACTGCTCTGGGTAGGAGTCGTC |
| | | | | GTCGTGCTGGCTAACGCAAGCCCCGGCAGAA |
| | | | | CCATAACAGTAGGGCCACGGGGGAATGCTTC |
| | | | | CAATGCTGCACCTTCCGCTTCACCGAGGAATG |
| | | | | CTTCTGCCCCAAGAACTACCCCCACTCCTCCT |
| | | | | CAACCCAGGAAAGCGACAAAGTCCAAGGCCA |
| | | | | GCACCGCAAAACCCGCTCCTCCTCCAAAGACT |
| | | | | GGGCCCCCAAAGACAAGTAGCGAACCAGTTC |
| | | | | GGTGCAACAGGCATGACCCACTTGCACGCTAT |
| | | | | GGGTCAAGAGTCCAGATACGGTGTCGCTTCCC |
| | | | | TAACAGTACAAGGACTGAGTTTCGGCTGCAG |
| | | | | ATCTGGCGTTATGCCACAGCTACTGACGCAGA |
| | | | | GATTGGTACCGCCCCCAGTTTGGAGGAAGTGA |
| | | | | TGGTCAACGTGTCCGCACCCCCAGGAGGACA |
| | | | | GCTGGTCTATGACTCAGCGCCCAATAGGACCG |
| | | | | ATCCCCACGTGATCTGGGCAGAAGGAGCCGG |
| | | | | TCCTGGGGCCTCTCCACGGCTGTACTCAGTTG |
| | | | | TTGGCCCGCTTGGACGACAGAGACTCATCATC |
| | | | | GAGGAACTGACACTGGAGACACAGGGGATGT |
| | | | | ACTACTGGGTGTGGGGCCGTACTGACCGCCCT |
| | | | | TCCGCATATGGCACTTGGGTGAGAGTTCGCGT |
| | | | | CTTTCGGCCCCCTTCTCTCACCATCCATCCTCA |
| | | | | TGCCGTGCTCGAAGGCCAGCCCTTTAAGGCCA |
| | | | | CATGCACTGCTGCGACCTACTACCCTGGCAAC |
| | | | | AGAGCCGAGTTTGTCTGGTTTGAGGATGGTCG |
| | | | | GCGAGTATTCGATCCAGCCCAGATTCACACAC |
| | | | | AAACGCAGGAAAATCCGGACGGCTTCAGCAC |
| | | | | AGTGTCCACGGTGACCTCTGCTGCAGTTGGTG |
| | | | | GACAAGGACCCCCTCGAACCTTCACCTGTCAG |
| | | | | CTGACCTGGCACAGAGACTCCGTAAGCTTCAG |
| | | | | CCGTAGAAACGCCTCTGGAACCGCCAGTGTGT |
| | | | | TGCCGAGGCCGACTATCACGATGGAATTCACA |
| | | | | GGCGATCATGCCGTCTGTACTGCCGGCTGTGT |
| | | | | GCCAGAAGGCGTAACCTTCGCTTGGTTTCTCG |
| | | | | GGGATGACTCAAGTCCTGCAGAGAAAGTGGC |
| | | | | TGTGGCCTCTCAGACGAGCTGCGGTCGACCAG |
| | | | | GAACAGCTACCATTCGCAGCACTCTGCCCGTG |
| | | | | TCCTACGAGCAGACGGAGTACATCTGCAGGCT |
| | | | | GGCCGGCTATCCCGATGGGATTCCAGTCCTGG |
| | | | | AGCACCACTGA |
| 311 | HSV-2 | HSV-2 | Version | ATGGCCTTGGGGAGAGTCGGCCTTGCAGTGG |
| | gC | gC | 2.2 | GACTGTGGGGTCTGCTGTGGGTTGGCGTGGTA |
| | | | | GTCGTGCTCGCTAACGCCAGCCCCGGCAGAAC |
| | | | | CATAACAGTAGGGCCACGGGGGAATGCTTCC |
| | | | | AATGCTGCACCTTCCGCTTCACCGAGGAATGC |
| | | | | TTCTGCCCCAAGAACTACCCCCACTCCTCCTC |
| | | | | AACCCAGGAAAGCGACAAAGTCCAAGGCCAG |
| | | | | CACCGCAAAACCCGCTCCTCCTCCAAAGACTG |
| | | | | GGCCCCCAAAGACAAGTAGCGAACCAGTTCG |
| | | | | GTGCAACAGGCATGACCCACTTGCACGCTATG |
| | | | | GGTCAAGAGTCCAGATACGGTGTCGCTTCCCT |
| | | | | AACAGTACAAGGACTGAGTTTCGGCTGCAGA |
| | | | | TCTGGCGTTATGCCACAGCTACTGACGCAGAG |
| | | | | ATTGGTACCGCCCCCAGTTTGGAGGAAGTGAT |
| | | | | GGTCAACGTGTCCGCACCCCCAGGAGGACAG |
| | | | | CTGGTCTATGACTCAGCGCCCAATAGGACCGA |
| | | | | TCCCCACGTGATCTGGGCAGAAGGAGCCGGT |
| | | | | CCTGGGGCCTCTCCACGGCTGTACTCAGTTGT |
| | | | | TGGCCCGCTTGGACGACAGAGACTCATCATCG |
| | | | | AGGAACTGACACTGGAGACACAGGGGATGTA |
| | | | | CTACTGGGTGTGGGGCCGTACTGACCGCCCTT |
| | | | | CCGCATATGGCACTTGGGTGAGAGTTCGCGTC |
| | | | | TTTCGGCCCCCTTCTCTCACCATCCATCCTCAT |
| | | | | GCCGTGCTCGAAGGCCAGCCCTTTAAGGCCAC |
| | | | | ATGCACTGCTGCGACCTACTACCCTGGCAACA |
| | | | | GAGCCGAGTTTGTCTGGTTTGAGGATGGTCGG |
| | | | | CGAGTATTCGATCCAGCCCAGATTCACACACA |
| | | | | AACGCAGGAAAATCCGGACGGCTTCAGCACA |
| | | | | GTGTCCACGGTGACCTCTGCTGCAGTTGGTGG |
| | | | | ACAAGGACCCCCTCGAACCTTCACCTGTCAGC |
| | | | | TGACCTGGCACAGAGACTCCGTAAGCTTCAGC |
| | | | | CGTAGAAACGCCTCTGGAACCGCCAGTGTGTT |
| | | | | GCCGAGGCCGACTATCACGATGGAATTCACA |
| | | | | GGCGATCATGCCGTCTGTACTGCCGGCTGTGT |
| | | | | GCCAGAAGGCGTAACCTTCGCTTGGTTTCTCG |
| | | | | GGGATGACTCAAGTCCTGCAGAGAAAGTGGC |
| | | | | TGTGGCCTCTCAGACGAGCTGCGGTCGACCAG |
| | | | | GAACAGCTACCATTCGCAGCACTCTGCCCGTG |
| | | | | TCCTACGAGCAGACGGAGTACATCTGCAGGCT |
| | | | | GGCCGGCTATCCCGATGGGATTCCAGTCCTGG |
| | | | | AGCACCACTGA |
| 312 | HSV-2 | HSV-2 | Version | ATGGCTCTGGGCAGAGTTGGACTGGCTGTTGG |
| | gC | gC | 2.3 | ACTGTGGGGACTGCTGTGGGTTGGAGTGGTGG |
| | | | | TGGTGCTGGCTAATGCTAGCCCCGGCAGAACC |
| | | | | ATAACAGTAGGGCCACGGGGGAATGCTTCCA |
| | | | | ATGCTGCACCTTCCGCTTCACCGAGGAATGCT |
| | | | | TCTGCCCCAAGAACTACCCCCACTCCTCCTCA |
| | | | | ACCCAGGAAAGCGACAAAGTCCAAGGCCAGC |
| | | | | ACCGCAAAACCCGCTCCTCCTCCAAAGACTGG |
| | | | | GCCCCCAAAGACAAGTAGCGAACCAGTTCGG |
| | | | | TGCAACAGGCATGACCCACTTGCACGCTATGG |
| | | | | GTCAAGAGTCCAGATACGGTGTCGCTTCCCTA |
| | | | | ACAGTACAAGGACTGAGTTTCGGCTGCAGATC |
| | | | | TGGCGTTATGCCACAGCTACTGACGCAGAGAT |
| | | | | TGGTACCGCCCCCAGTTTGGAGGAAGTGATGG |
| | | | | TCAACGTGTCCGCACCCCCAGGAGGACAGCT |
| | | | | GGTCTATGACTCAGCGCCCAATAGGACCGATC |
| | | | | CCCACGTGATCTGGGCAGAAGGAGCCGGTCC |
| | | | | TGGGGCCTCTCCACGGCTGTACTCAGTTGTTG |
| | | | | GCCCGCTTGGACGACAGAGACTCATCATCGA |
| | | | | GGAACTGACACTGGAGACACAGGGGATGTAC |
| | | | | TACTGGGTGTGGGGCCGTACTGACCGCCCTTC |
| | | | | CGCATATGGCACTTGGGTGAGAGTTCGCGTCT |
| | | | | TTCGGCCCCCTTCTCTCACCATCCATCCTCATG |
| | | | | CCGTGCTCGAAGGCCAGCCCTTTAAGGCCACA |
| | | | | TGCACTGCTGCGACCTACTACCCTGGCAACAG |
| | | | | AGCCGAGTTTGTCTGGTTTGAGGATGGTCGGC |
| | | | | GAGTATTCGATCCAGCCCAGATTCACACACAA |
| | | | | ACGCAGGAAAATCCGGACGGCTTCAGCACAG |
| | | | | TGTCCACGGTGACCTCTGCTGCAGTTGGTGGA |
| | | | | CAAGGACCCCCTCGAACCTTCACCTGTCAGCT |
| | | | | GACCTGGCACAGAGACTCCGTAAGCTTCAGCC |
| | | | | GTAGAAACGCCTCTGGAACCGCCAGTGTGTTG |
| | | | | CCGAGGCCGACTATCACGATGGAATTCACAG |
| | | | | GCGATCATGCCGTCTGTACTGCCGGCTGTGTG |
| | | | | CCAGAAGGCGTAACCTTCGCTTGGTTTCTCGG |
| | | | | GGATGACTCAAGTCCTGCAGAGAAAGTGGCT |
| | | | | GTGGCCTCTCAGACGAGCTGCGGTCGACCAG |
| | | | | GAACAGCTACCATTCGCAGCACTCTGCCCGTG |
| | | | | TCCTACGAGCAGACGGAGTACATCTGCAGGCT |
| | | | | GGCCGGCTATCCCGATGGGATTCCAGTCCTGG |
| | | | | AGCACCACTGA |
| 165 | HSV-1 | HSV-2 | Version | ATGGGCGGAGCTGCTGCTAGACTGGGAGCCG |
| | gD | gC | 1 | TGATCCTGTTCGTGGTTATCGTGGGACTGCAT |
| | | | | GGCGTGCGGGGCAAGTATAGCCCTGGCAGAA |
| | | | | CCATCACAGTGGGCCCTAGAGGCAACGCCTCT |
| | | | | AATGCCGCTCCTAGCGCCTCTCCTAGAAACGC |
| | | | | CTCTGCTCCCAGAACCACACCTACACCTCCAC |
| | | | | AGCCTAGAAAGGCCACCAAGAGCAAGGCCAG |
| | | | | CACAGCCAAACCTGCTCCTCCACCTAAGACAG |
| | | | | GCCCTCCAAAGACAAGCTCTGAGCCCGTGCG |
| | | | | GTGCAACAGACACGATCCACTGGCCAGATAC |
| | | | | GGCAGCCGGGTGCAGATCAGATGCAGATTCC |
| | | | | CCAACAGCACCCGGACCGAGTTCCGGCTCCA |
| | | | | GATTTGGAGATACGCCACCGCCACAGATGCC |
| | | | | GAGATTGGAACAGCCCCTAGCCTGGAAGAAG |
| | | | | TGATGGTCAACGTTTCAGCCCCTCCTGGCGGC |
| | | | | CAGCTGGTGTATGATTCTGCCCCTAACCGGAC |
| | | | | CGATCCTCACGTGATATGGGCTGAAGGTGCTG |
| | | | | GCCCTGGCGCTTCCCCTAGACTGTATTCTGTT |
| | | | | GTGGGCCCTCTGGGCAGACAGCGGCTGATCAT |
| | | | | TGAGGAACTGACCCTGGAAACCCAGGGCATG |
| | | | | TACTACTGGGTCTGGGGCAGAACCGATAGAC |
| | | | | CAAGCGCCTATGGCACCTGGGTTCGAGTGCGA |
| | | | | GTGTTCAGACCTCCTAGCCTGACCATCCATCC |
| | | | | TCACGCCGTTCTGGAAGGCCAGCCTTTCAAGG |
| | | | | CCACATGTACCGCCGCCACCTACTATCCCGGA |
| | | | | AACAGAGCCGAGTTCGTTTGGTTCGAGGACG |
| | | | | GCAGAAGGGTGTTCGACCCCGCTCAGATCCAC |
| | | | | ACACAGACCCAAGAGAACCCCGACGGCTTTA |
| | | | | GCACCGTGTCCACAGTGACATCTGCCGCCGTT |
| | | | | GGAGGACAGGGCCCTCCTAGAACCTTTACCTG |
| | | | | CCAGCTGACCTGGCACAGAGACAGCGTGTCCT |
| | | | | TCAGCAGAAGAAACGCCAGCGGCACAGCCAG |
| | | | | CGTTCTGCCTAGACCTACCATCACCATGGAAT |
| | | | | TCACCGGCGACCACGCCGTGTGTACAGCTGGA |
| | | | | TGTGTTCCTGAGGGCGTGACCTTCGCTTGGTT |
| | | | | TCTGGGCGACGATAGCAGCCCTGCCGAAAAA |
| | | | | GTGGCTGTGGCCAGCCAGACAAGCTGTGGCA |
| | | | | GACCTGGAACCGCCACCATCAGAAGCACACT |
| | | | | GCCTGTCAGCTACGAGCAGACCGAGTACATCT |
| | | | | GTCGGCTGGCCGGCTATCCTGATGGCATCCCT |
| | | | | GTGCTGGAACACCACTGATAA |
| 166 | HSV-1 | HSV-2 | Version | ATGGGAGGAGCAGCTGCCAGACTCGGTGCCG |
| | gD | gC | 2 | TGATCCTGTTCGTGGTCATTGTTGGCCTGCAC |
| | | | | GGGGTAAGGGGCAAGTACAGCCCCGGCAGAA |
| | | | | CCATAACAGTAGGGCCACGGGGGAATGCTTC |
| | | | | CAATGCTGCACCTTCCGCTTCACCGAGGAATG |
| | | | | CTTCTGCCCCAAGAACTACCCCCACTCCTCCT |
| | | | | CAACCCAGGAAAGCGACAAAGTCCAAGGCCA |
| | | | | GCACCGCAAAACCCGCTCCTCCTCCAAAGACT |
| | | | | GGGCCCCCAAAGACAAGTAGCGAACCAGTTC |
| | | | | GGTGCAACAGGCATGACCCACTTGCACGCTAT |
| | | | | GGGTCAAGAGTCCAGATACGGTGTCGCTTCCC |
| | | | | TAACAGTACAAGGACTGAGTTTCGGCTGCAG |
| | | | | ATCTGGCGTTATGCCACAGCTACTGACGCAGA |
| | | | | GATTGGTACCGCCCCCAGTTTGGAAGAGGTGA |
| | | | | TGGTCAACGTGTCCGCACCCCCAGGAGGACA |
| | | | | GCTGGTCTATGACTCAGCGCCCAATAGGACCG |
| | | | | ATCCCCACGTGATCTGGGCAGAAGGAGCCGG |
| | | | | TCCTGGGGCCTCTCCACGGCTGTACTCAGTTG |
| | | | | TTGGCCCGCTTGGACGACAGAGACTCATCATC |
| | | | | GAAGAGCTGACACTGGAGACACAGGGGATGT |
| | | | | ACTACTGGGTGTGGGGCCGTACTGACCGCCCT |
| | | | | TCCGCATATGGCACTTGGGTGAGAGTTCGCGT |
| | | | | CTTTCGGCCCCCTTCTCTCACCATCCATCCTCA |
| | | | | TGCCGTGCTCGAAGGCCAGCCCTTTAAGGCCA |
| | | | | CATGCACTGCTGCGACCTACTACCCTGGCAAC |
| | | | | AGAGCCGAGTTTGTCTGGTTTGAGGATGGTCG |
| | | | | GCGAGTATTCGATCCAGCCCAGATTCACACAC |
| | | | | AAACGCAGGAAAATCCGGACGGCTTCAGCAC |
| | | | | AGTGTCCACGGTGACCTCTGCTGCAGTTGGTG |
| | | | | GACAAGGACCCCCTCGAACCTTCACCTGTCAG |
| | | | | CTGACCTGGCACAGAGACTCCGTAAGCTTCAG |
| | | | | CCGTAGAAACGCCTCTGGAACCGCCAGTGTGT |
| | | | | TGCCGAGGCCGACTATCACGATGGAATTCACA |
| | | | | GGCGATCATGCCGTCTGTACTGCCGGCTGTGT |
| | | | | GCCAGAAGGCGTAACCTTCGCTTGGTTTCTCG |
| | | | | GGGATGACTCAAGTCCTGCAGAGAAAGTGGC |
| | | | | TGTGGCCTCTCAGACGAGCTGCGGTCGACCAG |
| | | | | GAACAGCTACCATTCGCAGCACTCTGCCCGTG |
| | | | | TCCTACGAGCAGACGGAGTACATCTGCAGGCT |
| | | | | GGCCGGCTATCCCGATGGGATTCCAGTCCTGG |
| | | | | AGCACCACTGATAA |
| 342 | HSV-1 | HSV-2 | Version | ATGGGCGGCGCAGCCGCTAGACTGGGTGCAG |
| | gD | gC | 2.1 | TCATCCTCTTTGTGGTGATCGTGGGGCTGCAT |
| | | | | GGGGTCAGAGGGAAGTATAGCCCCGGCAGAA |
| | | | | CCATAACAGTAGGGCCACGGGGGAATGCTTC |
| | | | | CAATGCTGCACCTTCCGCTTCACCGAGGAATG |
| | | | | CTTCTGCCCCAAGAACTACCCCCACTCCTCCT |
| | | | | CAACCCAGGAAAGCGACAAAGTCCAAGGCCA |
| | | | | GCACCGCAAAACCCGCTCCTCCTCCAAAGACT |
| | | | | GGGCCCCCAAAGACAAGTAGCGAACCAGTTC |
| | | | | GGTGCAACAGGCATGACCCACTTGCACGCTAT |
| | | | | GGGTCAAGAGTCCAGATACGGTGTCGCTTCCC |
| | | | | TAACAGTACAAGGACTGAGTTTCGGCTGCAG |
| | | | | ATCTGGCGTTATGCCACAGCTACTGACGCAGA |
| | | | | GATTGGTACCGCCCCCAGTTTGGAGGAAGTGA |
| | | | | TGGTCAACGTGTCCGCACCCCCAGGAGGACA |
| | | | | GCTGGTCTATGACTCAGCGCCCAATAGGACCG |
| | | | | ATCCCCACGTGATCTGGGCAGAAGGAGCCGG |
| | | | | TCCTGGGGCCTCTCCACGGCTGTACTCAGTTG |
| | | | | TTGGCCCGCTTGGACGACAGAGACTCATCATC |
| | | | | GAGGAACTGACACTGGAGACACAGGGGATGT |
| | | | | ACTACTGGGTGTGGGGCCGTACTGACCGCCCT |
| | | | | TCCGCATATGGCACTTGGGTGAGAGTTCGCGT |
| | | | | CTTTCGGCCCCCTTCTCTCACCATCCATCCTCA |
| | | | | TGCCGTGCTCGAAGGCCAGCCCTTTAAGGCCA |
| | | | | CATGCACTGCTGCGACCTACTACCCTGGCAAC |
| | | | | AGAGCCGAGTTTGTCTGGTTTGAGGATGGTCG |
| | | | | GCGAGTATTCGATCCAGCCCAGATTCACACAC |
| | | | | AAACGCAGGAAAATCCGGACGGCTTCAGCAC |
| | | | | AGTGTCCACGGTGACCTCTGCTGCAGTTGGTG |
| | | | | GACAAGGACCCCCTCGAACCTTCACCTGTCAG |
| | | | | CTGACCTGGCACAGAGACTCCGTAAGCTTCAG |
| | | | | CCGTAGAAACGCCTCTGGAACCGCCAGTGTGT |
| | | | | TGCCGAGGCCGACTATCACGATGGAATTCACA |
| | | | | GGCGATCATGCCGTCTGTACTGCCGGCTGTGT |
| | | | | GCCAGAAGGCGTAACCTTCGCTTGGTTTCTCG |
| | | | | GGGATGACTCAAGTCCTGCAGAGAAAGTGGC |
| | | | | TGTGGCCTCTCAGACGAGCTGCGGTCGACCAG |
| | | | | GAACAGCTACCATTCGCAGCACTCTGCCCGTG |
| | | | | TCCTACGAGCAGACGGAGTACATCTGCAGGCT |
| | | | | GGCCGGCTATCCCGATGGGATTCCAGTCCTGG |
| | | | | AGCACCACTGATAA |
| 343 | HSV-1 | HSV-2 | Version | ATGGGAGGAGCAGCTGCCAGACTCGGTGCCG |
| | gD | gC | 2.2 | TGATCCTGTTCGTGGTCATTGTTGGCCTGCAC |
| | | | | GGGGTAAGGGGCAAGTACAGCCCCGGCAGAA |
| | | | | CCATAACAGTAGGGCCACGGGGGAATGCTTC |
| | | | | CAATGCTGCACCTTCCGCTTCACCGAGGAATG |
| | | | | CTTCTGCCCCAAGAACTACCCCCACTCCTCCT |
| | | | | CAACCCAGGAAAGCGACAAAGTCCAAGGCCA |
| | | | | GCACCGCAAAACCCGCTCCTCCTCCAAAGACT |
| | | | | GGGCCCCCAAAGACAAGTAGCGAACCAGTTC |
| | | | | GGTGCAACAGGCATGACCCACTTGCACGCTAT |
| | | | | GGGTCAAGAGTCCAGATACGGTGTCGCTTCCC |
| | | | | TAACAGTACAAGGACTGAGTTTCGGCTGCAG |
| | | | | ATCTGGCGTTATGCCACAGCTACTGACGCAGA |
| | | | | GATTGGTACCGCCCCCAGTTTGGAGGAAGTGA |
| | | | | TGGTCAACGTGTCCGCACCCCCAGGAGGACA |
| | | | | GCTGGTCTATGACTCAGCGCCCAATAGGACCG |
| | | | | ATCCCCACGTGATCTGGGCAGAAGGAGCCGG |
| | | | | TCCTGGGGCCTCTCCACGGCTGTACTCAGTTG |
| | | | | TTGGCCCGCTTGGACGACAGAGACTCATCATC |
| | | | | GAGGAACTGACACTGGAGACACAGGGGATGT |
| | | | | ACTACTGGGTGTGGGGCCGTACTGACCGCCCT |
| | | | | TCCGCATATGGCACTTGGGTGAGAGTTCGCGT |
| | | | | CTTTCGGCCCCCTTCTCTCACCATCCATCCTCA |
| | | | | TGCCGTGCTCGAAGGCCAGCCCTTTAAGGCCA |
| | | | | CATGCACTGCTGCGACCTACTACCCTGGCAAC |
| | | | | AGAGCCGAGTTTGTCTGGTTTGAGGATGGTCG |
| | | | | GCGAGTATTCGATCCAGCCCAGATTCACACAC |
| | | | | AAACGCAGGAAAATCCGGACGGCTTCAGCAC |
| | | | | AGTGTCCACGGTGACCTCTGCTGCAGTTGGTG |
| | | | | GACAAGGACCCCCTCGAACCTTCACCTGTCAG |
| | | | | CTGACCTGGCACAGAGACTCCGTAAGCTTCAG |
| | | | | CCGTAGAAACGCCTCTGGAACCGCCAGTGTGT |
| | | | | TGCCGAGGCCGACTATCACGATGGAATTCACA |
| | | | | GGCGATCATGCCGTCTGTACTGCCGGCTGTGT |
| | | | | GCCAGAAGGCGTAACCTTCGCTTGGTTTCTCG |
| | | | | GGGATGACTCAAGTCCTGCAGAGAAAGTGGC |
| | | | | TGTGGCCTCTCAGACGAGCTGCGGTCGACCAG |
| | | | | GAACAGCTACCATTCGCAGCACTCTGCCCGTG |
| | | | | TCCTACGAGCAGACGGAGTACATCTGCAGGCT |
| | | | | GGCCGGCTATCCCGATGGGATTCCAGTCCTGG |
| | | | | AGCACCACTGATAA |
| 167 | HSV-1 | HSV-2 | Version | ATGGGAGGAGCTGCTGCTAGATTGGGAGCTG |
| | gD | gC | 3 | TGATTCTGTTTGTGGTGATTGTGGGACTGCAT |
| | | | | GGAGTGAGAGGAAAATACTCTCCTGGAAGAA |
| | | | | CCATCACAGTGGGACCAAGAGGAAATGCAAG |
| | | | | CAATGCAGCACCTTCTGCTTCTCCAAGAAATG |
| | | | | CTTCTGCTCCAAGAACCACCCCAACCCCTCCT |
| | | | | CAGCCAAGAAAAGCAACCAAATCCAAAGCAT |
| | | | | CCACAGCAAAACCTGCACCTCCTCCAAAAAC |
| | | | | AGGACCTCCAAAAACCTCCTCTGAACCTGTGA |
| | | | | GATGCAACAGACATGATCCTCTGGCAAGATAT |
| | | | | GGATCAAGAGTGCAGATCAGATGCAGATTTC |
| | | | | CAAATTCCACCAGAACAGAATTCAGACTCCA |
| | | | | GATCTGGAGATATGCAACAGCAACAGATGCA |
| | | | | GAAATTGGAACAGCACCATCTCTGGAAGAAG |
| | | | | TGATGGTGAATGTGTCTGCTCCTCCTGGAGGA |
| | | | | CAGCTGGTGTATGATTCTGCTCCAAACAGAAC |
| | | | | AGATCCTCATGTGATCTGGGCTGAAGGAGCTG |
| | | | | GACCTGGAGCTTCTCCAAGACTGTACTCTGTG |
| | | | | GTGGGACCTCTGGGAAGACAGAGACTGATCA |
| | | | | TTGAAGAACTGACCCTGGAAACCCAGGGAAT |
| | | | | GTACTACTGGGTGTGGGGAAGAACAGACAGA |
| | | | | CCTTCTGCTTATGGAACCTGGGTGAGAGTGAG |
| | | | | AGTGTTCAGACCTCCTTCTCTGACCATCCACC |
| | | | | CTCATGCTGTGCTGGAAGGACAGCCTTTCAAA |
| | | | | GCAACCTGCACAGCAGCAACCTACTACCCTGG |
| | | | | AAACAGAGCTGAATTTGTGTGGTTTGAAGATG |
| | | | | GAAGAAGGGTGTTTGATCCTGCTCAGATCCAC |
| | | | | ACCCAGACCCAGGAAAATCCTGATGGATTTTC |
| | | | | CACAGTGTCCACAGTGACATCTGCTGCTGTGG |
| | | | | GAGGACAGGGACCTCCAAGAACCTTCACCTG |
| | | | | CCAGCTGACCTGGCACAGAGATTCTGTGTCTT |
| | | | | TTTCAAGAAGAAATGCTTCTGGAACAGCTTCT |
| | | | | GTGCTGCCAAGACCAACCATCACCATGGAATT |
| | | | | CACAGGAGATCATGCTGTGTGCACAGCTGGAT |
| | | | | GTGTGCCTGAAGGAGTGACCTTTGCTTGGTTT |
| | | | | CTGGGAGATGATTCTTCTCCAGCTGAAAAAGT |
| | | | | GGCTGTGGCTTCCCAGACCTCTTGTGGAAGAC |
| | | | | CTGGAACAGCAACCATCAGATCCACCCTGCCT |
| | | | | GTGTCTTATGAACAGACAGAATACATTTGCAG |
| | | | | ACTGGCTGGATACCCTGATGGAATCCCTGTGC |
| | | | | TGGAACACCACTGATAA |
| 168 | HSV-1 | HSV-2 | Version | ATGGGCGGCGCAGCCGCTAGACTGGGTGCAG |
| | gD | gC | 4 | TCATCCTCTTTGTGGTGATCGTGGGGCTGCAT |
| | | | | GGGGTCAGAGGGAAGTATTCTCCGGGACGGA |
| | | | | CTATAACCGTAGGTCCAAGAGGAAACGCCTCT |
| | | | | AACGCAGCCCCGTCTGCCTCACCACGAAACGC |
| | | | | CTCAGCTCCCAGAACCACTCCTACTCCACCCC |
| | | | | AGCCTAGGAAGGCGACGAAATCCAAGGCTTC |
| | | | | CACGGCCAAACCCGCCCCTCCACCCAAAACC |
| | | | | GGACCTCCTAAGACCAGCTCTGAACCGGTGCG |
| | | | | GTGTAATAGGCACGACCCATTGGCGCGATATG |
| | | | | GCAGTAGGGTCCAGATACGGTGCAGATTCCC |
| | | | | AAACAGCACAAGAACAGAATTCCGGCTGCAA |
| | | | | ATCTGGCGATATGCAACGGCCACCGATGCCG |
| | | | | AAATCGGAACAGCACCCAGTCTGGAAGAAGT |
| | | | | GATGGTGAACGTCAGTGCTCCACCTGGCGGAC |
| | | | | AACTGGTGTACGACTCTGCACCCAATCGCACA |
| | | | | GATCCCCACGTGATTTGGGCCGAGGGTGCTGG |
| | | | | ACCTGGGGCTTCACCCAGGCTGTATAGCGTTG |
| | | | | TAGGGCCACTTGGGAGGCAGAGACTCATCATT |
| | | | | GAGGAACTGACCCTGGAAACTCAGGGCATGT |
| | | | | ACTACTGGGTATGGGGCCGCACAGATCGCCCC |
| | | | | AGCGCTTATGGCACCTGGGTGCGGGTGCGGGT |
| | | | | GTTTCGCCCACCCTCCCTCACCATTCACCCTCA |
| | | | | TGCGGTTCTGGAGGGACAGCCTTTCAAGGCAA |
| | | | | CTTGTACCGCAGCCACCTACTATCCCGGCAAT |
| | | | | AGAGCGGAGTTCGTCTGGTTTGAGGACGGCC |
| | | | | GTAGGGTGTTCGATCCTGCCCAGATTCACACC |
| | | | | CAGACACAGGAGAATCCCGACGGCTTTAGCA |
| | | | | CAGTGAGCACTGTGACGTCTGCTGCCGTTGGT |
| | | | | GGTCAAGGGCCTCCTCGTACCTTCACATGCCA |
| | | | | ATTGACCTGGCACCGCGACTCAGTTAGCTTTA |
| | | | | GCCGCCGGAATGCCAGTGGGACCGCCAGTGT |
| | | | | TCTCCCAAGGCCGACAATCACCATGGAGTTCA |
| | | | | CTGGCGACCATGCAGTGTGCACAGCTGGGTGT |
| | | | | GTCCCAGAAGGCGTGACTTTCGCCTGGTTTCT |
| | | | | GGGTGATGACTCCTCACCCGCCGAGAAAGTA |
| | | | | GCTGTCGCTTCCCAGACTTCCTGTGGACGTCC |
| | | | | TGGAACTGCGACAATCCGAAGCACACTGCCG |
| | | | | GTTTCCTACGAGCAGACGGAGTACATATGCCG |
| | | | | CCTTGCAGGCTACCCCGATGGAATTCCAGTCC |
| | | | | TTGAGCACCATTGA |
| 169 | HSV-1 | HSV-2 | Version | ATGCATCAGGGCGCTCCATCTTGGGGTAGACG |
| | gB | gC | 1 | TTGGTTCGTTGTGTGGGCCCTGCTGGGACTGA |
| | | | | CACTGGGAGTTCTGGTTGCCTCTGCTGCTCCT |
| | | | | AGCCCTGGCAGAACCATCACAGTGGGCCCTA |
| | | | | GAGGCAACGCCTCTAATGCCGCTCCTAGCGCC |
| | | | | TCTCCTAGAAACGCCTCTGCTCCCAGAACCAC |
| | | | | ACCTACACCTCCACAGCCTAGAAAGGCCACC |
| | | | | AAGAGCAAGGCCAGCACAGCCAAACCTGCTC |
| | | | | CTCCACCTAAGACAGGCCCTCCAAAGACAAG |
| | | | | CTCTGAGCCCGTGCGGTGCAACAGACACGATC |
| | | | | CACTGGCCAGATACGGCAGCCGGGTGCAGAT |
| | | | | CAGATGCAGATTCCCCAACAGCACCCGGACC |
| | | | | GAGTTCCGGCTCCAGATTTGGAGATACGCCAC |
| | | | | CGCCACAGATGCCGAGATTGGAACAGCCCCT |
| | | | | AGCCTGGAAGAAGTGATGGTCAACGTTTCAG |
| | | | | CCCCTCCTGGCGGCCAGCTGGTGTATGATTCT |
| | | | | GCCCCTAACCGGACCGATCCTCACGTGATATG |
| | | | | GGCTGAAGGTGCTGGCCCTGGCGCTTCCCCTA |
| | | | | GACTGTATTCTGTTGTGGGCCCTCTGGGCAGA |
| | | | | CAGCGGCTGATCATTGAGGAACTGACCCTGG |
| | | | | AAACCCAGGGCATGTACTACTGGGTCTGGGG |
| | | | | CAGAACCGATAGACCAAGCGCCTATGGCACC |
| | | | | TGGGTTCGAGTGCGAGTGTTCAGACCTCCTAG |
| | | | | CCTGACCATCCATCCTCACGCCGTTCTGGAAG |
| | | | | GCCAGCCTTTCAAGGCCACATGTACCGCCGCC |
| | | | | ACCTACTATCCCGGAAACAGAGCCGAGTTCGT |
| | | | | TTGGTTCGAGGACGGCAGAAGGGTGTTCGAC |
| | | | | CCCGCTCAGATCCACACACAGACCCAAGAGA |
| | | | | ACCCCGACGGCTTTAGCACCGTGTCCACAGTG |
| | | | | ACATCTGCCGCCGTTGGAGGACAGGGCCCTCC |
| | | | | TAGAACCTTTACCTGCCAGCTGACCTGGCACA |
| | | | | GAGACAGCGTGTCCTTCAGCAGAAGAAACGC |
| | | | | CAGCGGCACAGCCAGCGTTCTGCCTAGACCTA |
| | | | | CCATCACCATGGAATTCACCGGCGACCACGCC |
| | | | | GTGTGTACAGCTGGATGTGTTCCTGAGGGCGT |
| | | | | GACCTTCGCTTGGTTTCTGGGCGACGATAGCA |
| | | | | GCCCTGCCGAAAAAGTGGCTGTGGCCAGCCA |
| | | | | GACAAGCTGTGGCAGACCTGGAACCGCCACC |
| | | | | ATCAGAAGCACACTGCCTGTCAGCTACGAGC |
| | | | | AGACCGAGTACATCTGTCGGCTGGCCGGCTAT |
| | | | | CCTGATGGCATCCCTGTGCTGGAACACCACTG |
| | | | | ATAA |
| 170 | HSV-1 | HSV-2 | Version | ATGCACCAGGGTGCCCCTTCCTGGGGCAGAA |
| | gB | gC | 2 | GGTGGTTCGTGGTGTGGGCCCTTCTGGGGCTG |
| | | | | ACCCTCGGAGTCCTGGTTGCGAGCGCAGCTCC |
| | | | | CAGCCCCGGCAGAACCATAACAGTAGGGCCA |
| | | | | CGGGGGAATGCTTCCAATGCTGCACCTTCCGC |
| | | | | TTCACCGAGGAATGCTTCTGCCCCAAGAACTA |
| | | | | CCCCCACTCCTCCTCAACCCAGGAAAGCGACA |
| | | | | AAGTCCAAGGCCAGCACCGCAAAACCCGCTC |
| | | | | CTCCTCCAAAGACTGGGCCCCCAAAGACAAG |
| | | | | TAGCGAACCAGTTCGGTGCAACAGGCATGAC |
| | | | | CCACTTGCACGCTATGGGTCAAGAGTCCAGAT |
| | | | | ACGGTGTCGCTTCCCTAACAGTACAAGGACTG |
| | | | | AGTTTCGGCTGCAGATCTGGCGTTATGCCACA |
| | | | | GCTACTGACGCAGAGATTGGTACCGCCCCCAG |
| | | | | TTTGGAAGAGGTGATGGTCAACGTGTCCGCAC |
| | | | | CCCCAGGAGGACAGCTGGTCTATGACTCAGC |
| | | | | GCCCAATAGGACCGATCCCCACGTGATCTGGG |
| | | | | CAGAAGGAGCCGGTCCTGGGGCCTCTCCACG |
| | | | | GCTGTACTCAGTTGTTGGCCCGCTTGGACGAC |
| | | | | AGAGACTCATCATCGAAGAGCTGACACTGGA |
| | | | | GACACAGGGGATGTACTACTGGGTGTGGGGC |
| | | | | CGTACTGACCGCCCTTCCGCATATGGCACTTG |
| | | | | GGTGAGAGTTCGCGTCTTTCGGCCCCCTTCTC |
| | | | | TCACCATCCATCCTCATGCCGTGCTCGAAGGC |
| | | | | CAGCCCTTTAAGGCCACATGCACTGCTGCGAC |
| | | | | CTACTACCCTGGCAACAGAGCCGAGTTTGTCT |
| | | | | GGTTTGAGGATGGTCGGCGAGTATTCGATCCA |
| | | | | GCCCAGATTCACACACAAACGCAGGAAAATC |
| | | | | CGGACGGCTTCAGCACAGTGTCCACGGTGACC |
| | | | | TCTGCTGCAGTTGGTGGACAAGGACCCCCTCG |
| | | | | AACCTTCACCTGTCAGCTGACCTGGCACAGAG |
| | | | | ACTCCGTAAGCTTCAGCCGTAGAAACGCCTCT |
| | | | | GGAACCGCCAGTGTGTTGCCGAGGCCGACTAT |
| | | | | CACGATGGAATTCACAGGCGATCATGCCGTCT |
| | | | | GTACTGCCGGCTGTGTGCCAGAAGGCGTAACC |
| | | | | TTCGCTTGGTTTCTCGGGGATGACTCAAGTCC |
| | | | | TGCAGAGAAAGTGGCTGTGGCCTCTCAGACG |
| | | | | AGCTGCGGTCGACCAGGAACAGCTACCATTC |
| | | | | GCAGCACTCTGCCCGTGTCCTACGAGCAGACG |
| | | | | GAGTACATCTGCAGGCTGGCCGGCTATCCCGA |
| | | | | TGGGATTCCAGTCCTGGAGCACCACTGATAA |
| 344 | HSV-1 | HSV-2 | Version | ATGCATCAAGGCGCACCCAGTTGGGGCAGAC |
| | gB | gC | 2.1 | GGTGGTTCGTAGTGTGGGCCTTGCTGGGACTG |
| | | | | ACTCTCGGTGTCCTCGTTGCGTCTGCTGCACC |
| | | | | GAGCCCCGGCAGAACCATAACAGTAGGGCCA |
| | | | | CGGGGGAATGCTTCCAATGCTGCACCTTCCGC |
| | | | | TTCACCGAGGAATGCTTCTGCCCCAAGAACTA |
| | | | | CCCCCACTCCTCCTCAACCCAGGAAAGCGACA |
| | | | | AAGTCCAAGGCCAGCACCGCAAAACCCGCTC |
| | | | | CTCCTCCAAAGACTGGGCCCCCAAAGACAAG |
| | | | | TAGCGAACCAGTTCGGTGCAACAGGCATGAC |
| | | | | CCACTTGCACGCTATGGGTCAAGAGTCCAGAT |
| | | | | ACGGTGTCGCTTCCCTAACAGTACAAGGACTG |
| | | | | AGTTTCGGCTGCAGATCTGGCGTTATGCCACA |
| | | | | GCTACTGACGCAGAGATTGGTACCGCCCCCAG |
| | | | | TTTGGAGGAAGTGATGGTCAACGTGTCCGCAC |
| | | | | CCCCAGGAGGACAGCTGGTCTATGACTCAGC |
| | | | | GCCCAATAGGACCGATCCCCACGTGATCTGGG |
| | | | | CAGAAGGAGCCGGTCCTGGGGCCTCTCCACG |
| | | | | GCTGTACTCAGTTGTTGGCCCGCTTGGACGAC |
| | | | | AGAGACTCATCATCGAGGAACTGACACTGGA |
| | | | | GACACAGGGGATGTACTACTGGGTGTGGGGC |
| | | | | CGTACTGACCGCCCTTCCGCATATGGCACTTG |
| | | | | GGTGAGAGTTCGCGTCTTTCGGCCCCCTTCTC |
| | | | | TCACCATCCATCCTCATGCCGTGCTCGAAGGC |
| | | | | CAGCCCTTTAAGGCCACATGCACTGCTGCGAC |
| | | | | CTACTACCCTGGCAACAGAGCCGAGTTTGTCT |
| | | | | GGTTTGAGGATGGTCGGCGAGTATTCGATCCA |
| | | | | GCCCAGATTCACACACAAACGCAGGAAAATC |
| | | | | CGGACGGCTTCAGCACAGTGTCCACGGTGACC |
| | | | | TCTGCTGCAGTTGGTGGACAAGGACCCCCTCG |
| | | | | AACCTTCACCTGTCAGCTGACCTGGCACAGAG |
| | | | | ACTCCGTAAGCTTCAGCCGTAGAAACGCCTCT |
| | | | | GGAACCGCCAGTGTGTTGCCGAGGCCGACTAT |
| | | | | CACGATGGAATTCACAGGCGATCATGCCGTCT |
| | | | | GTACTGCCGGCTGTGTGCCAGAAGGCGTAACC |
| | | | | TTCGCTTGGTTTCTCGGGGATGACTCAAGTCC |
| | | | | TGCAGAGAAAGTGGCTGTGGCCTCTCAGACG |
| | | | | AGCTGCGGTCGACCAGGAACAGCTACCATTC |
| | | | | GCAGCACTCTGCCCGTGTCCTACGAGCAGACG |
| | | | | GAGTACATCTGCAGGCTGGCCGGCTATCCCGA |
| | | | | TGGGATTCCAGTCCTGGAGCACCACTGATAA |
| 171 | HSV-1 | HSV-2 | Version | ATGCACCAGGGAGCACCTTCTTGGGGAAGAA |
| | gB | gC | 3 | GATGGTTTGTGGTGTGGGCTCTGCTGGGACTG |
| | | | | ACCCTGGGAGTGCTGGTGGCTTCTGCTGCTCC |
| | | | | TTCTCCTGGAAGAACCATCACAGTGGGACCAA |
| | | | | GAGGAAATGCAAGCAATGCAGCACCTTCTGC |
| | | | | TTCTCCAAGAAATGCTTCTGCTCCAAGAACCA |
| | | | | CCCCAACCCCTCCTCAGCCAAGAAAAGCAAC |
| | | | | CAAATCCAAAGCATCCACAGCAAAACCTGCA |
| | | | | CCTCCTCCAAAAACAGGACCTCCAAAAACCTC |
| | | | | CTCTGAACCTGTGAGATGCAACAGACATGATC |
| | | | | CTCTGGCAAGATATGGATCAAGAGTGCAGAT |
| | | | | CAGATGCAGATTTCCAAATTCCACCAGAACAG |
| | | | | AATTCAGACTCCAGATCTGGAGATATGCAACA |
| | | | | GCAACAGATGCAGAAATTGGAACAGCACCAT |
| | | | | CTCTGGAAGAAGTGATGGTGAATGTGTCTGCT |
| | | | | CCTCCTGGAGGACAGCTGGTGTATGATTCTGC |
| | | | | TCCAAACAGAACAGATCCTCATGTGATCTGGG |
| | | | | CTGAAGGAGCTGGACCTGGAGCTTCTCCAAG |
| | | | | ACTGTACTCTGTGGTGGGACCTCTGGGAAGAC |
| | | | | AGAGACTGATCATTGAAGAACTGACCCTGGA |
| | | | | AACCCAGGGAATGTACTACTGGGTGTGGGGA |
| | | | | AGAACAGACAGACCTTCTGCTTATGGAACCTG |
| | | | | GGTGAGAGTGAGAGTGTTCAGACCTCCTTCTC |
| | | | | TGACCATCCACCCTCATGCTGTGCTGGAAGGA |
| | | | | CAGCCTTTCAAAGCAACCTGCACAGCAGCAA |
| | | | | CCTACTACCCTGGAAACAGAGCTGAATTTGTG |
| | | | | TGGTTTGAAGATGGAAGAAGGGTGTTTGATCC |
| | | | | TGCTCAGATCCACACCCAGACCCAGGAAAAT |
| | | | | CCTGATGGATTTTCCACAGTGTCCACAGTGAC |
| | | | | ATCTGCTGCTGTGGGAGGACAGGGACCTCCA |
| | | | | AGAACCTTCACCTGCCAGCTGACCTGGCACAG |
| | | | | AGATTCTGTGTCTTTTTCAAGAAGAAATGCTT |
| | | | | CTGGAACAGCTTCTGTGCTGCCAAGACCAACC |
| | | | | ATCACCATGGAATTCACAGGAGATCATGCTGT |
| | | | | GTGCACAGCTGGATGTGTGCCTGAAGGAGTG |
| | | | | ACCTTTGCTTGGTTTCTGGGAGATGATTCTTCT |
| | | | | CCAGCTGAAAAAGTGGCTGTGGCTTCCCAGAC |
| | | | | CTCTTGTGGAAGACCTGGAACAGCAACCATCA |
| | | | | GATCCACCCTGCCTGTGTCTTATGAACAGACA |
| | | | | GAATACATTTGCAGACTGGCTGGATACCCTGA |
| | | | | TGGAATCCCTGTGCTGGAACACCACTGATAA |
| 172 | HSV-1 | HSV-2 | Version | ATGCATCAAGGCGCACCCAGTTGGGGCAGAC |
| | gB | gC | 4 | GGTGGTTCGTAGTGTGGGCCTTGCTGGGACTG |
| | | | | ACTCTCGGTGTCCTCGTTGCGTCTGCTGCACC |
| | | | | GAGTCCAGGAAGGACGATTACGGTGGGACCC |
| | | | | AGAGGTAATGCGTCCAATGCTGCGCCATCCGC |
| | | | | TTCTCCACGGAACGCATCTGCACCCAGGACTA |
| | | | | CACCGACACCACCTCAGCCGCGCAAAGCCAC |
| | | | | CAAGAGCAAGGCCAGCACAGCCAAACCCGCT |
| | | | | CCTCCACCTAAAACCGGACCACCTAAGACCA |
| | | | | GCTCTGAACCCGTCAGATGCAACAGGCACGA |
| | | | | TCCGTTGGCCAGATATGGCAGTCGCGTCCAGA |
| | | | | TCAGGTGTCGCTTCCCTAACAGCACACGGACC |
| | | | | GAGTTCAGGCTGCAAATTTGGCGCTACGCTAC |
| | | | | AGCCACTGACGCAGAGATTGGCACTGCTCCCA |
| | | | | GTCTGGAGGAGGTCATGGTGAACGTGTCTGCT |
| | | | | CCACCAGGCGGTCAGCTGGTCTATGACTCAGC |
| | | | | CCCTAATCGCACAGATCCTCACGTGATTTGGG |
| | | | | CAGAAGGTGCGGGGCCTGGGGCCTCCCCAAG |
| | | | | GCTCTACTCAGTGGTTGGACCCCTTGGGAGAC |
| | | | | AGCGGCTGATCATCGAGGAACTGACTCTCGA |
| | | | | AACCCAAGGTATGTACTACTGGGTATGGGGC |
| | | | | AGAACAGACAGACCTTCAGCTTATGGCACCTG |
| | | | | GGTGCGGGTGAGAGTGTTTAGGCCTCCCTCCC |
| | | | | TGACGATCCATCCCCATGCTGTGCTGGAAGGA |
| | | | | CAGCCGTTCAAGGCAACATGCACAGCAGCCA |
| | | | | CTTACTATCCCGGAAACCGTGCTGAGTTTGTG |
| | | | | TGGTTCGAGGATGGGCGACGTGTATTCGACCC |
| | | | | TGCCCAGATTCACACCCAGACACAGGAGAAT |
| | | | | CCCGACGGGTTTTCCACTGTGAGCACCGTGAC |
| | | | | ATCAGCGGCAGTAGGAGGGCAGGGCCCACCC |
| | | | | CGAACGTTCACTTGCCAGCTTACTTGGCATCG |
| | | | | GGACAGTGTTAGCTTTAGCCGCCGGAATGCCT |
| | | | | CTGGCACCGCATCCGTCCTTCCTCGCCCAACC |
| | | | | ATCACCATGGAATTCACTGGCGATCACGCCGT |
| | | | | TTGTACAGCCGGGTGTGTTCCCGAGGGAGTGA |
| | | | | CCTTTGCTTGGTTTCTGGGCGATGACTCAAGC |
| | | | | CCAGCCGAAAAGGTGGCCGTCGCCTCCCAAA |
| | | | | CGAGCTGTGGGCGACCTGGCACCGCTACCATA |
| | | | | CGTAGCACTCTGCCCGTTTCCTACGAACAGAC |
| | | | | CGAGTATATCTGCCGATTGGCCGGTTACCCCG |
| | | | | ATGGGATACCAGTCCTGGAGCACCACTGA |
| 173 | HSV-2 gI | HSV-2 | Version | ATGCCTGGCAGATCTCTGCAAGGACTGGCCAT |
| | | gC | 1 | CCTCGGACTGTGGGTTTGCGCAACAGGACTGA |
| | | | | GCCCTGGCAGAACCATCACAGTGGGCCCTAG |
| | | | | AGGCAACGCCTCTAATGCCGCTCCTAGCGCCT |
| | | | | CTCCTAGAAACGCCTCTGCTCCCAGAACCACA |
| | | | | CCTACACCTCCACAGCCTAGAAAGGCCACCA |
| | | | | AGAGCAAGGCCAGCACAGCCAAACCTGCTCC |
| | | | | TCCACCTAAGACAGGCCCTCCAAAGACAAGC |
| | | | | TCTGAGCCCGTGCGGTGCAACAGACACGATCC |
| | | | | ACTGGCCAGATACGGCAGCCGGGTGCAGATC |
| | | | | AGATGCAGATTCCCCAACAGCACCCGGACCG |
| | | | | AGTTCCGGCTCCAGATTTGGAGATACGCCACC |
| | | | | GCCACAGATGCCGAGATTGGAACAGCCCCTA |
| | | | | GCCTGGAAGAAGTGATGGTCAACGTTTCAGCC |
| | | | | CCTCCTGGCGGCCAGCTGGTGTATGATTCTGC |
| | | | | CCCTAACCGGACCGATCCTCACGTGATATGGG |
| | | | | CTGAAGGTGCTGGCCCTGGCGCTTCCCCTAGA |
| | | | | CTGTATTCTGTTGTGGGCCCTCTGGGCAGACA |
| | | | | GCGGCTGATCATTGAGGAACTGACCCTGGAA |
| | | | | ACCCAGGGCATGTACTACTGGGTCTGGGGCA |
| | | | | GAACCGATAGACCAAGCGCCTATGGCACCTG |
| | | | | GGTTCGAGTGCGAGTGTTCAGACCTCCTAGCC |
| | | | | TGACCATCCATCCTCACGCCGTTCTGGAAGGC |
| | | | | CAGCCTTTCAAGGCCACATGTACCGCCGCCAC |
| | | | | CTACTATCCCGGAAACAGAGCCGAGTTCGTTT |
| | | | | GGTTCGAGGACGGCAGAAGGGTGTTCGACCC |
| | | | | CGCTCAGATCCACACACAGACCCAAGAGAAC |
| | | | | CCCGACGGCTTTAGCACCGTGTCCACAGTGAC |
| | | | | ATCTGCCGCCGTTGGAGGACAGGGCCCTCCTA |
| | | | | GAACCTTTACCTGCCAGCTGACCTGGCACAGA |
| | | | | GACAGCGTGTCCTTCAGCAGAAGAAACGCCA |
| | | | | GCGGCACAGCCAGCGTTCTGCCTAGACCTACC |
| | | | | ATCACCATGGAATTCACCGGCGACCACGCCGT |
| | | | | GTGTACAGCTGGATGTGTTCCTGAGGGCGTGA |
| | | | | CCTTCGCTTGGTTTCTGGGCGACGATAGCAGC |
| | | | | CCTGCCGAAAAAGTGGCTGTGGCCAGCCAGA |
| | | | | CAAGCTGTGGCAGACCTGGAACCGCCACCAT |
| | | | | CAGAAGCACACTGCCTGTCAGCTACGAGCAG |
| | | | | ACCGAGTACATCTGTCGGCTGGCCGGCTATCC |
| | | | | TGATGGCATCCCTGTGCTGGAACACCACTGAT |
| | | | | AA |
| 174 | HSV-2 gI | HSV-2 | Version | ATGCCCGGCAGAAGCCTCCAGGGACTGGCTA |
| | | gC | 2 | TCCTGGGGCTGTGGGTGTGCGCCACCGGTCTT |
| | | | | AGCCCCGGCAGAACCATAACAGTAGGGCCAC |
| | | | | GGGGGAATGCTTCCAATGCTGCACCTTCCGCT |
| | | | | TCACCGAGGAATGCTTCTGCCCCAAGAACTAC |
| | | | | CCCCACTCCTCCTCAACCCAGGAAAGCGACAA |
| | | | | AGTCCAAGGCCAGCACCGCAAAACCCGCTCC |
| | | | | TCCTCCAAAGACTGGGCCCCCAAAGACAAGT |
| | | | | AGCGAACCAGTTCGGTGCAACAGGCATGACC |
| | | | | CACTTGCACGCTATGGGTCAAGAGTCCAGATA |
| | | | | CGGTGTCGCTTCCCTAACAGTACAAGGACTGA |
| | | | | GTTTCGGCTGCAGATCTGGCGTTATGCCACAG |
| | | | | CTACTGACGCAGAGATTGGTACCGCCCCCAGT |
| | | | | TTGGAAGAGGTGATGGTCAACGTGTCCGCACC |
| | | | | CCCAGGAGGACAGCTGGTCTATGACTCAGCG |
| | | | | CCCAATAGGACCGATCCCCACGTGATCTGGGC |
| | | | | AGAAGGAGCCGGTCCTGGGGCCTCTCCACGG |
| | | | | CTGTACTCAGTTGTTGGCCCGCTTGGACGACA |
| | | | | GAGACTCATCATCGAAGAGCTGACACTGGAG |
| | | | | ACACAGGGGATGTACTACTGGGTGTGGGGCC |
| | | | | GTACTGACCGCCCTTCCGCATATGGCACTTGG |
| | | | | GTGAGAGTTCGCGTCTTTCGGCCCCCTTCTCTC |
| | | | | ACCATCCATCCTCATGCCGTGCTCGAAGGCCA |
| | | | | GCCCTTTAAGGCCACATGCACTGCTGCGACCT |
| | | | | ACTACCCTGGCAACAGAGCCGAGTTTGTCTGG |
| | | | | TTTGAGGATGGTCGGCGAGTATTCGATCCAGC |
| | | | | CCAGATTCACACACAAACGCAGGAAAATCCG |
| | | | | GACGGCTTCAGCACAGTGTCCACGGTGACCTC |
| | | | | TGCTGCAGTTGGTGGACAAGGACCCCCTCGAA |
| | | | | CCTTCACCTGTCAGCTGACCTGGCACAGAGAC |
| | | | | TCCGTAAGCTTCAGCCGTAGAAACGCCTCTGG |
| | | | | AACCGCCAGTGTGTTGCCGAGGCCGACTATCA |
| | | | | CGATGGAATTCACAGGCGATCATGCCGTCTGT |
| | | | | ACTGCCGGCTGTGTGCCAGAAGGCGTAACCTT |
| | | | | CGCTTGGTTTCTCGGGGATGACTCAAGTCCTG |
| | | | | CAGAGAAAGTGGCTGTGGCCTCTCAGACGAG |
| | | | | CTGCGGTCGACCAGGAACAGCTACCATTCGCA |
| | | | | GCACTCTGCCCGTGTCCTACGAGCAGACGGAG |
| | | | | TACATCTGCAGGCTGGCCGGCTATCCCGATGG |
| | | | | GATTCCAGTCCTGGAGCACCACTGATAA |
| 175 | HSV-2 gI | HSV-2 | Version | ATGCCTGGAAGATCTCTCCAGGGACTGGCAAT |
| | | gC | 3 | CCTGGGACTGTGGGTGTGTGCAACAGGACTGT |
| | | | | CTCCTGGAAGAACCATCACAGTGGGACCAAG |
| | | | | AGGAAATGCAAGCAATGCAGCACCTTCTGCTT |
| | | | | CTCCAAGAAATGCTTCTGCTCCAAGAACCACC |
| | | | | CCAACCCCTCCTCAGCCAAGAAAAGCAACCA |
| | | | | AATCCAAAGCATCCACAGCAAAACCTGCACC |
| | | | | TCCTCCAAAAACAGGACCTCCAAAAACCTCCT |
| | | | | CTGAACCTGTGAGATGCAACAGACATGATCCT |
| | | | | CTGGCAAGATATGGATCAAGAGTGCAGATCA |
| | | | | GATGCAGATTTCCAAATTCCACCAGAACAGA |
| | | | | ATTCAGACTCCAGATCTGGAGATATGCAACAG |
| | | | | CAACAGATGCAGAAATTGGAACAGCACCATC |
| | | | | TCTGGAAGAAGTGATGGTGAATGTGTCTGCTC |
| | | | | CTCCTGGAGGACAGCTGGTGTATGATTCTGCT |
| | | | | CCAAACAGAACAGATCCTCATGTGATCTGGGC |
| | | | | TGAAGGAGCTGGACCTGGAGCTTCTCCAAGA |
| | | | | CTGTACTCTGTGGTGGGACCTCTGGGAAGACA |
| | | | | GAGACTGATCATTGAAGAACTGACCCTGGAA |
| | | | | ACCCAGGGAATGTACTACTGGGTGTGGGGAA |
| | | | | GAACAGACAGACCTTCTGCTTATGGAACCTGG |
| | | | | GTGAGAGTGAGAGTGTTCAGACCTCCTTCTCT |
| | | | | GACCATCCACCCTCATGCTGTGCTGGAAGGAC |
| | | | | AGCCTTTCAAAGCAACCTGCACAGCAGCAAC |
| | | | | CTACTACCCTGGAAACAGAGCTGAATTTGTGT |
| | | | | GGTTTGAAGATGGAAGAAGGGTGTTTGATCCT |
| | | | | GCTCAGATCCACACCCAGACCCAGGAAAATC |
| | | | | CTGATGGATTTTCCACAGTGTCCACAGTGACA |
| | | | | TCTGCTGCTGTGGGAGGACAGGGACCTCCAA |
| | | | | GAACCTTCACCTGCCAGCTGACCTGGCACAGA |
| | | | | GATTCTGTGTCTTTTTCAAGAAGAAATGCTTC |
| | | | | TGGAACAGCTTCTGTGCTGCCAAGACCAACCA |
| | | | | TCACCATGGAATTCACAGGAGATCATGCTGTG |
| | | | | TGCACAGCTGGATGTGTGCCTGAAGGAGTGA |
| | | | | CCTTTGCTTGGTTTCTGGGAGATGATTCTTCTC |
| | | | | CAGCTGAAAAAGTGGCTGTGGCTTCCCAGACC |
| | | | | TCTTGTGGAAGACCTGGAACAGCAACCATCA |
| | | | | GATCCACCCTGCCTGTGTCTTATGAACAGACA |
| | | | | GAATACATTTGCAGACTGGCTGGATACCCTGA |
| | | | | TGGAATCCCTGTGCTGGAACACCACTGATAA |
| 176 | HSV-2 gI | HSV-2 | Version | ATGCCTGGGAGAAGCCTGCAAGGGCTCGCAA |
| | | gC | 4 | TCTTGGGCCTGTGGGTTTGTGCCACAGGCTTG |
| | | | | TCCCCGGGTCGAACAATCACTGTTGGGCCCAG |
| | | | | GGGAAATGCCAGCAATGCTGCACCTTCAGCA |
| | | | | AGCCCACGAAACGCTTCAGCACCCAGGACAA |
| | | | | CACCCACTCCACCTCAACCGCGGAAAGCCACC |
| | | | | AAGAGCAAGGCAAGTACCGCCAAACCCGCTC |
| | | | | CTCCTCCCAAGACAGGGCCACCCAAGACCTCT |
| | | | | AGTGAGCCAGTGAGGTGTAACCGCCATGATC |
| | | | | CCCTTGCCAGATACGGGAGCAGAGTGCAGAT |
| | | | | TAGGTGCCGGTTTCCAAACTCCACGAGAACCG |
| | | | | AATTTCGCCTCCAGATTTGGCGGTATGCGACT |
| | | | | GCCACAGACGCAGAGATTGGTACCGCTCCCA |
| | | | | GCCTGGAGGAGGTCATGGTGAACGTGTCAGC |
| | | | | GCCTCCGGGTGGCCAGCTGGTCTACGACTCTG |
| | | | | CCCCAAATCGAACCGACCCTCACGTCATCTGG |
| | | | | GCTGAAGGAGCGGGACCAGGAGCCTCTCCAC |
| | | | | GCTTGTATAGCGTAGTTGGCCCTCTGGGGAGA |
| | | | | CAGCGCCTGATCATTGAGGAACTGACCCTTGA |
| | | | | GACACAGGGGATGTACTACTGGGTGTGGGGC |
| | | | | AGGACTGACAGGCCCAGTGCCTATGGAACTT |
| | | | | GGGTTAGGGTCCGCGTCTTTCGGCCACCCAGT |
| | | | | CTGACCATCCATCCACATGCCGTGCTGGAAGG |
| | | | | CCAGCCCTTCAAAGCGACTTGCACTGCCGCCA |
| | | | | CGTACTATCCAGGGAATAGAGCCGAGTTCGTT |
| | | | | TGGTTCGAGGATGGCCGGAGAGTATTCGATCC |
| | | | | AGCTCAGATCCACACCCAGACGCAGGAAAAC |
| | | | | CCGGACGGCTTTAGCACGGTGAGTACCGTCAC |
| | | | | CTCTGCTGCCGTCGGAGGCCAAGGACCTCCCC |
| | | | | GTACCTTCACATGCCAGCTTACATGGCACCGG |
| | | | | GACTCAGTAAGCTTTTCACGTCGTAATGCATC |
| | | | | CGGTACTGCTTCTGTGCTGCCTCGACCCACCA |
| | | | | TCACCATGGAGTTCACAGGGGATCACGCAGT |
| | | | | GTGTACGGCAGGCTGCGTGCCTGAAGGCGTG |
| | | | | ACATTCGCCTGGTTTCTCGGTGATGACTCCTCT |
| | | | | CCTGCTGAAAAGGTGGCTGTAGCCTCCCAAAC |
| | | | | AAGCTGTGGTCGGCCTGGAACTGCCACTATAC |
| | | | | GCTCCACTCTCCCGGTGTCCTACGAACAGACC |
| | | | | GAGTACATATGCAGACTGGCTGGATATCCCGA |
| | | | | TGGCATTCCCGTGCTGGAGCATCACTGA |
| 177 | HSV-2 | HSV-2 | Version | ATGGCTAGAGGTGCCGGCCTGGTGTTCTTTGT |
| | gE | gC | 1 | TGGCGTGTGGGTCGTGTCCTGTCTGGCCGCTG |
| | | | | CTCCTAGAACATCTAGCCCTGGCAGAACCATC |
| | | | | ACAGTGGGCCCTAGAGGCAACGCCTCTAATG |
| | | | | CCGCTCCTAGCGCCTCTCCTAGAAACGCCTCT |
| | | | | GCTCCCAGAACCACACCTACACCTCCACAGCC |
| | | | | TAGAAAGGCCACCAAGAGCAAGGCCAGCACA |
| | | | | GCCAAACCTGCTCCTCCACCTAAGACAGGCCC |
| | | | | TCCAAAGACAAGCTCTGAGCCCGTGCGGTGC |
| | | | | AACAGACACGATCCACTGGCCAGATACGGCA |
| | | | | GCCGGGTGCAGATCAGATGCAGATTCCCCAA |
| | | | | CAGCACCCGGACCGAGTTCCGGCTCCAGATTT |
| | | | | GGAGATACGCCACCGCCACAGATGCCGAGAT |
| | | | | TGGAACAGCCCCTAGCCTGGAAGAAGTGATG |
| | | | | GTCAACGTTTCAGCCCCTCCTGGCGGCCAGCT |
| | | | | GGTGTATGATTCTGCCCCTAACCGGACCGATC |
| | | | | CTCACGTGATATGGGCTGAAGGTGCTGGCCCT |
| | | | | GGCGCTTCCCCTAGACTGTATTCTGTTGTGGG |
| | | | | CCCTCTGGGCAGACAGCGGCTGATCATTGAGG |
| | | | | AACTGACCCTGGAAACCCAGGGCATGTACTA |
| | | | | CTGGGTCTGGGGCAGAACCGATAGACCAAGC |
| | | | | GCCTATGGCACCTGGGTTCGAGTGCGAGTGTT |
| | | | | CAGACCTCCTAGCCTGACCATCCATCCTCACG |
| | | | | CCGTTCTGGAAGGCCAGCCTTTCAAGGCCACA |
| | | | | TGTACCGCCGCCACCTACTATCCCGGAAACAG |
| | | | | AGCCGAGTTCGTTTGGTTCGAGGACGGCAGA |
| | | | | AGGGTGTTCGACCCCGCTCAGATCCACACACA |
| | | | | GACCCAAGAGAACCCCGACGGCTTTAGCACC |
| | | | | GTGTCCACAGTGACATCTGCCGCCGTTGGAGG |
| | | | | ACAGGGCCCTCCTAGAACCTTTACCTGCCAGC |
| | | | | TGACCTGGCACAGAGACAGCGTGTCCTTCAGC |
| | | | | AGAAGAAACGCCAGCGGCACAGCCAGCGTTC |
| | | | | TGCCTAGACCTACCATCACCATGGAATTCACC |
| | | | | GGCGACCACGCCGTGTGTACAGCTGGATGTGT |
| | | | | TCCTGAGGGCGTGACCTTCGCTTGGTTTCTGG |
| | | | | GCGACGATAGCAGCCCTGCCGAAAAAGTGGC |
| | | | | TGTGGCCAGCCAGACAAGCTGTGGCAGACCT |
| | | | | GGAACCGCCACCATCAGAAGCACACTGCCTG |
| | | | | TCAGCTACGAGCAGACCGAGTACATCTGTCGG |
| | | | | CTGGCCGGCTATCCTGATGGCATCCCTGTGCT |
| | | | | GGAACACCACTGATAA |
| 178 | HSV-2 | HSV-2 | Version | ATGGCGAGAGGAGCCGGGCTCGTGTTCTTTGT |
| | gE | gC | 2 | GGGCGTATGGGTCGTTTCCTGCCTGGCTGCCG |
| | | | | CACCCAGGACCAGCAGCCCCGGCAGAACCAT |
| | | | | AACAGTAGGGCCACGGGGGAATGCTTCCAAT |
| | | | | GCTGCACCTTCCGCTTCACCGAGGAATGCTTC |
| | | | | TGCCCCAAGAACTACCCCCACTCCTCCTCAAC |
| | | | | CCAGGAAAGCGACAAAGTCCAAGGCCAGCAC |
| | | | | CGCAAAACCCGCTCCTCCTCCAAAGACTGGGC |
| | | | | CCCCAAAGACAAGTAGCGAACCAGTTCGGTG |
| | | | | CAACAGGCATGACCCACTTGCACGCTATGGGT |
| | | | | CAAGAGTCCAGATACGGTGTCGCTTCCCTAAC |
| | | | | AGTACAAGGACTGAGTTTCGGCTGCAGATCTG |
| | | | | GCGTTATGCCACAGCTACTGACGCAGAGATTG |
| | | | | GTACCGCCCCCAGTTTGGAAGAGGTGATGGTC |
| | | | | AACGTGTCCGCACCCCCAGGAGGACAGCTGG |
| | | | | TCTATGACTCAGCGCCCAATAGGACCGATCCC |
| | | | | CACGTGATCTGGGCAGAAGGAGCCGGTCCTG |
| | | | | GGGCCTCTCCACGGCTGTACTCAGTTGTTGGC |
| | | | | CCGCTTGGACGACAGAGACTCATCATCGAAG |
| | | | | AGCTGACACTGGAGACACAGGGGATGTACTA |
| | | | | CTGGGTGTGGGGCCGTACTGACCGCCCTTCCG |
| | | | | CATATGGCACTTGGGTGAGAGTTCGCGTCTTT |
| | | | | CGGCCCCCTTCTCTCACCATCCATCCTCATGCC |
| | | | | GTGCTCGAAGGCCAGCCCTTTAAGGCCACATG |
| | | | | CACTGCTGCGACCTACTACCCTGGCAACAGAG |
| | | | | CCGAGTTTGTCTGGTTTGAGGATGGTCGGCGA |
| | | | | GTATTCGATCCAGCCCAGATTCACACACAAAC |
| | | | | GCAGGAAAATCCGGACGGCTTCAGCACAGTG |
| | | | | TCCACGGTGACCTCTGCTGCAGTTGGTGGACA |
| | | | | AGGACCCCCTCGAACCTTCACCTGTCAGCTGA |
| | | | | CCTGGCACAGAGACTCCGTAAGCTTCAGCCGT |
| | | | | AGAAACGCCTCTGGAACCGCCAGTGTGTTGCC |
| | | | | GAGGCCGACTATCACGATGGAATTCACAGGC |
| | | | | GATCATGCCGTCTGTACTGCCGGCTGTGTGCC |
| | | | | AGAAGGCGTAACCTTCGCTTGGTTTCTCGGGG |
| | | | | ATGACTCAAGTCCTGCAGAGAAAGTGGCTGT |
| | | | | GGCCTCTCAGACGAGCTGCGGTCGACCAGGA |
| | | | | ACAGCTACCATTCGCAGCACTCTGCCCGTGTC |
| | | | | CTACGAGCAGACGGAGTACATCTGCAGGCTG |
| | | | | GCCGGCTATCCCGATGGGATTCCAGTCCTGGA |
| | | | | GCACCACTGATAA |
| 345 | HSV-2 | HSV-2 | Version | AGCCCCGGCAGAACCATAACAGTAGGGCCAC |
| | gE | gC | 2.1 | GGGGGAATGCTTCCAATGCTGCACCTTCCGCT |
| | | | | TCACCGAGGAATGCTTCTGCCCCAAGAACTAC |
| | | | | CCCCACTCCTCCTCAACCCAGGAAAGCGACAA |
| | | | | AGTCCAAGGCCAGCACCGCAAAACCCGCTCC |
| | | | | TCCTCCAAAGACTGGGCCCCCAAAGACAAGT |
| | | | | AGCGAACCAGTTCGGTGCAACAGGCATGACC |
| | | | | CACTTGCACGCTATGGGTCAAGAGTCCAGATA |
| | | | | CGGTGTCGCTTCCCTAACAGTACAAGGACTGA |
| | | | | GTTTCGGCTGCAGATCTGGCGTTATGCCACAG |
| | | | | CTACTGACGCAGAGATTGGTACCGCCCCCAGT |
| | | | | TTGGAGGAAGTGATGGTCAACGTGTCCGCACC |
| | | | | CCCAGGAGGACAGCTGGTCTATGACTCAGCG |
| | | | | CCCAATAGGACCGATCCCCACGTGATCTGGGC |
| | | | | AGAAGGAGCCGGTCCTGGGGCCTCTCCACGG |
| | | | | CTGTACTCAGTTGTTGGCCCGCTTGGACGACA |
| | | | | GAGACTCATCATCGAGGAACTGACACTGGAG |
| | | | | ACACAGGGGATGTACTACTGGGTGTGGGGCC |
| | | | | GTACTGACCGCCCTTCCGCATATGGCACTTGG |
| | | | | GTGAGAGTTCGCGTCTTTCGGCCCCCTTCTCTC |
| | | | | ACCATCCATCCTCATGCCGTGCTCGAAGGCCA |
| | | | | GCCCTTTAAGGCCACATGCACTGCTGCGACCT |
| | | | | ACTACCCTGGCAACAGAGCCGAGTTTGTCTGG |
| | | | | TTTGAGGATGGTCGGCGAGTATTCGATCCAGC |
| | | | | CCAGATTCACACACAAACGCAGGAAAATCCG |
| | | | | GACGGCTTCAGCACAGTGTCCACGGTGACCTC |
| | | | | TGCTGCAGTTGGTGGACAAGGACCCCCTCGAA |
| | | | | CCTTCACCTGTCAGCTGACCTGGCACAGAGAC |
| | | | | TCCGTAAGCTTCAGCCGTAGAAACGCCTCTGG |
| | | | | AACCGCCAGTGTGTTGCCGAGGCCGACTATCA |
| | | | | CGATGGAATTCACAGGCGATCATGCCGTCTGT |
| | | | | ACTGCCGGCTGTGTGCCAGAAGGCGTAACCTT |
| | | | | CGCTTGGTTTCTCGGGGATGACTCAAGTCCTG |
| | | | | CAGAGAAAGTGGCTGTGGCCTCTCAGACGAG |
| | | | | CTGCGGTCGACCAGGAACAGCTACCATTCGCA |
| | | | | GCACTCTGCCCGTGTCCTACGAGCAGACGGAG |
| | | | | TACATCTGCAGGCTGGCCGGCTATCCCGATGG |
| | | | | GATTCCAGTCCTGGAGCACCACTGATAA |
| 179 | HSV-2 | HSV-2 | Version | ATGGCAAGAGGAGCAGGACTGGTGTTTTTCGT |
| | gE | gC | 3 | GGGAGTTTGGGTGGTGTCTTGCCTGGCTGCTG |
| | | | | CTCCAAGAACCTCTTCTCCTGGAAGAACCATC |
| | | | | ACAGTGGGACCAAGAGGAAATGCAAGCAATG |
| | | | | CAGCACCTTCTGCTTCTCCAAGAAATGCTTCT |
| | | | | GCTCCAAGAACCACCCCAACCCCTCCTCAGCC |
| | | | | AAGAAAAGCAACCAAATCCAAAGCATCCACA |
| | | | | GCAAAACCTGCACCTCCTCCAAAAACAGGAC |
| | | | | CTCCAAAAACCTCCTCTGAACCTGTGAGATGC |
| | | | | AACAGACATGATCCTCTGGCAAGATATGGATC |
| | | | | AAGAGTGCAGATCAGATGCAGATTTCCAAATT |
| | | | | CCACCAGAACAGAATTCAGACTCCAGATCTG |
| | | | | GAGATATGCAACAGCAACAGATGCAGAAATT |
| | | | | GGAACAGCACCATCTCTGGAAGAAGTGATGG |
| | | | | TGAATGTGTCTGCTCCTCCTGGAGGACAGCTG |
| | | | | GTGTATGATTCTGCTCCAAACAGAACAGATCC |
| | | | | TCATGTGATCTGGGCTGAAGGAGCTGGACCTG |
| | | | | GAGCTTCTCCAAGACTGTACTCTGTGGTGGGA |
| | | | | CCTCTGGGAAGACAGAGACTGATCATTGAAG |
| | | | | AACTGACCCTGGAAACCCAGGGAATGTACTA |
| | | | | CTGGGTGTGGGGAAGAACAGACAGACCTTCT |
| | | | | GCTTATGGAACCTGGGTGAGAGTGAGAGTGTT |
| | | | | CAGACCTCCTTCTCTGACCATCCACCCTCATG |
| | | | | CTGTGCTGGAAGGACAGCCTTTCAAAGCAACC |
| | | | | TGCACAGCAGCAACCTACTACCCTGGAAACA |
| | | | | GAGCTGAATTTGTGTGGTTTGAAGATGGAAGA |
| | | | | AGGGTGTTTGATCCTGCTCAGATCCACACCCA |
| | | | | GACCCAGGAAAATCCTGATGGATTTTCCACAG |
| | | | | TGTCCACAGTGACATCTGCTGCTGTGGGAGGA |
| | | | | CAGGGACCTCCAAGAACCTTCACCTGCCAGCT |
| | | | | GACCTGGCACAGAGATTCTGTGTCTTTTTCAA |
| | | | | GAAGAAATGCTTCTGGAACAGCTTCTGTGCTG |
| | | | | CCAAGACCAACCATCACCATGGAATTCACAG |
| | | | | GAGATCATGCTGTGTGCACAGCTGGATGTGTG |
| | | | | CCTGAAGGAGTGACCTTTGCTTGGTTTCTGGG |
| | | | | AGATGATTCTTCTCCAGCTGAAAAAGTGGCTG |
| | | | | TGGCTTCCCAGACCTCTTGTGGAAGACCTGGA |
| | | | | ACAGCAACCATCAGATCCACCCTGCCTGTGTC |
| | | | | TTATGAACAGACAGAATACATTTGCAGACTGG |
| | | | | CTGGATACCCTGATGGAATCCCTGTGCTGGAA |
| | | | | CACCACTGATAA |
| 180 | HSV-2 | HSV-2 | Version | ATGGCGAGAGGAGCCGGACTCGTGTTCTTTGT |
| | gE | gC | 4 | GGGAGTCTGGGTTGTGAGCTGCCTGGCAGCA |
| | | | | GCTCCACGCACTAGCTCTCCAGGCAGAACTAT |
| | | | | CACAGTGGGACCCAGAGGGAATGCCAGCAAT |
| | | | | GCAGCCCCGAGTGCCAGCCCTCGTAACGCCA |
| | | | | GCGCTCCCAGAACAACCCCAACTCCACCGCA |
| | | | | GCCTAGAAAGGCGACCAAGTCCAAAGCATCC |
| | | | | ACTGCAAAACCAGCCCCACCTCCCAAAACGG |
| | | | | GACCTCCCAAGACCAGCTCCGAGCCTGTAAG |
| | | | | GTGCAATCGGCATGACCCCTTGGCCCGATATG |
| | | | | GCAGTCGCGTGCAGATTCGATGTCGGTTTCCC |
| | | | | AACTCTACCCGGACTGAGTTCCGGTTGCAGAT |
| | | | | CTGGAGGTATGCGACCGCCACTGACGCTGAG |
| | | | | ATCGGCACAGCACCAAGCCTGGAAGAAGTGA |
| | | | | TGGTGAACGTTAGTGCTCCTCCGGGGGGCAA |
| | | | | CTCGTGTATGACTCCGCACCCAACCGCACAGA |
| | | | | TCCTCACGTGATTTGGGCCGAAGGAGCCGGAC |
| | | | | CCGGTGCGTCACCTAGGCTCTACTCTGTCGTA |
| | | | | GGACCACTGGGCCGTCAACGCCTGATAATCG |
| | | | | AGGAGCTGACTCTGGAGACACAGGGTATGTA |
| | | | | CTACTGGGTCTGGGGCAGAACCGACAGGCCA |
| | | | | TCTGCTTACGGGACATGGGTCCGCGTTCGAGT |
| | | | | ATTTCGGCCACCCTCACTGACCATACATCCCC |
| | | | | ATGCCGTTCTTGAAGGGCAGCCTTTCAAGGCA |
| | | | | ACCTGTACTGCTGCCACATACTATCCCGGGAA |
| | | | | TAGGGCCGAGTTCGTCTGGTTTGAAGATGGCC |
| | | | | GAAGGGTGTTTGACCCGGCTCAGATCCACACC |
| | | | | CAGACACAGGAGAACCCCGATGGCTTCAGTA |
| | | | | CGGTGTCTACCGTCACAAGCGCCGCTGTGGGT |
| | | | | GGCCAAGGTCCTCCCAGAACTTTCACCTGTCA |
| | | | | GCTGACGTGGCACAGGGATTCCGTGAGCTTTT |
| | | | | CCCGCCGCAATGCGTCAGGGACCGCCTCCGTG |
| | | | | CTTCCTCGGCCAACCATCACAATGGAATTCAC |
| | | | | GGGTGATCACGCTGTCTGCACAGCTGGCTGCG |
| | | | | TTCCTGAGGGCGTGACATTCGCATGGTTTCTT |
| | | | | GGTGACGACTCATCTCCCGCAGAGAAGGTGG |
| | | | | CTGTTGCCTCACAAACGAGTTGTGGGCGTCCA |
| | | | | GGCACTGCCACCATTCGGTCCACCCTCCCCGT |
| | | | | AAGCTACGAACAGACTGAGTATATTTGCAGA |
| | | | | CTGGCTGGATACCCGGATGGGATTCCTGTCCT |
| | | | | GGAACATCACTGA |
| 181 | EboZ | HSV-2 | Version | ATGGGAGTGACCGGCATTCTCCAGCTGCCTCG |
| | | gC | 1 | GGACAGATTCAAGCGGACCAGCTTCTTCCTGT |
| | | | | GGGTCATCATCCTGTTCCAGCGGACCTTCAGC |
| | | | | ATCCCCAGCCCTGGCAGAACCATCACAGTGG |
| | | | | GCCCTAGAGGCAACGCCTCTAATGCCGCTCCT |
| | | | | AGCGCCTCTCCTAGAAACGCCTCTGCTCCCAG |
| | | | | AACCACACCTACACCTCCACAGCCTAGAAAG |
| | | | | GCCACCAAGAGCAAGGCCAGCACAGCCAAAC |
| | | | | CTGCTCCTCCACCTAAGACAGGCCCTCCAAAG |
| | | | | ACAAGCTCTGAGCCCGTGCGGTGCAACAGAC |
| | | | | ACGATCCACTGGCCAGATACGGCAGCCGGGT |
| | | | | GCAGATCAGATGCAGATTCCCCAACAGCACC |
| | | | | CGGACCGAGTTCCGGCTCCAGATTTGGAGATA |
| | | | | CGCCACCGCCACAGATGCCGAGATTGGAACA |
| | | | | GCCCCTAGCCTGGAAGAAGTGATGGTCAACG |
| | | | | TTTCAGCCCCTCCTGGCGGCCAGCTGGTGTAT |
| | | | | GATTCTGCCCCTAACCGGACCGATCCTCACGT |
| | | | | GATATGGGCTGAAGGTGCTGGCCCTGGCGCTT |
| | | | | CCCCTAGACTGTATTCTGTTGTGGGCCCTCTG |
| | | | | GGCAGACAGCGGCTGATCATTGAGGAACTGA |
| | | | | CCCTGGAAACCCAGGGCATGTACTACTGGGTC |
| | | | | TGGGGCAGAACCGATAGACCAAGCGCCTATG |
| | | | | GCACCTGGGTTCGAGTGCGAGTGTTCAGACCT |
| | | | | CCTAGCCTGACCATCCATCCTCACGCCGTTCT |
| | | | | GGAAGGCCAGCCTTTCAAGGCCACATGTACC |
| | | | | GCCGCCACCTACTATCCCGGAAACAGAGCCG |
| | | | | AGTTCGTTTGGTTCGAGGACGGCAGAAGGGT |
| | | | | GTTCGACCCCGCTCAGATCCACACACAGACCC |
| | | | | AAGAGAACCCCGACGGCTTTAGCACCGTGTCC |
| | | | | ACAGTGACATCTGCCGCCGTTGGAGGACAGG |
| | | | | GCCCTCCTAGAACCTTTACCTGCCAGCTGACC |
| | | | | TGGCACAGAGACAGCGTGTCCTTCAGCAGAA |
| | | | | GAAACGCCAGCGGCACAGCCAGCGTTCTGCC |
| | | | | TAGACCTACCATCACCATGGAATTCACCGGCG |
| | | | | ACCACGCCGTGTGTACAGCTGGATGTGTTCCT |
| | | | | GAGGGCGTGACCTTCGCTTGGTTTCTGGGCGA |
| | | | | CGATAGCAGCCCTGCCGAAAAAGTGGCTGTG |
| | | | | GCCAGCCAGACAAGCTGTGGCAGACCTGGAA |
| | | | | CCGCCACCATCAGAAGCACACTGCCTGTCAGC |
| | | | | TACGAGCAGACCGAGTACATCTGTCGGCTGGC |
| | | | | CGGCTATCCTGATGGCATCCCTGTGCTGGAAC |
| | | | | ACCACTGATAA |
| 182 | EboZ | HSV-2 | Version | ATGGGAGTGACTGGCATACTCCAGCTTCCTAG |
| | | gC | 2 | AGACAGGTTTAAGCGCACATCCTTCTTTCTGT |
| | | | | GGGTCATCATCCTGTTCCAACGGACCTTCAGC |
| | | | | ATTCCCAGCCCCGGCAGAACCATAACAGTAG |
| | | | | GGCCACGGGGGAATGCTTCCAATGCTGCACCT |
| | | | | TCCGCTTCACCGAGGAATGCTTCTGCCCCAAG |
| | | | | AACTACCCCCACTCCTCCTCAACCCAGGAAAG |
| | | | | CGACAAAGTCCAAGGCCAGCACCGCAAAACC |
| | | | | CGCTCCTCCTCCAAAGACTGGGCCCCCAAAGA |
| | | | | CAAGTAGCGAACCAGTTCGGTGCAACAGGCA |
| | | | | TGACCCACTTGCACGCTATGGGTCAAGAGTCC |
| | | | | AGATACGGTGTCGCTTCCCTAACAGTACAAGG |
| | | | | ACTGAGTTTCGGCTGCAGATCTGGCGTTATGC |
| | | | | CACAGCTACTGACGCAGAGATTGGTACCGCCC |
| | | | | CCAGTTTGGAAGAGGTGATGGTCAACGTGTCC |
| | | | | GCACCCCCAGGAGGACAGCTGGTCTATGACTC |
| | | | | AGCGCCCAATAGGACCGATCCCCACGTGATCT |
| | | | | GGGCAGAAGGAGCCGGTCCTGGGGCCTCTCC |
| | | | | ACGGCTGTACTCAGTTGTTGGCCCGCTTGGAC |
| | | | | GACAGAGACTCATCATCGAAGAGCTGACACT |
| | | | | GGAGACACAGGGGATGTACTACTGGGTGTGG |
| | | | | GGCCGTACTGACCGCCCTTCCGCATATGGCAC |
| | | | | TTGGGTGAGAGTTCGCGTCTTTCGGCCCCCTT |
| | | | | CTCTCACCATCCATCCTCATGCCGTGCTCGAA |
| | | | | GGCCAGCCCTTTAAGGCCACATGCACTGCTGC |
| | | | | GACCTACTACCCTGGCAACAGAGCCGAGTTTG |
| | | | | TCTGGTTTGAGGATGGTCGGCGAGTATTCGAT |
| | | | | CCAGCCCAGATTCACACACAAACGCAGGAAA |
| | | | | ATCCGGACGGCTTCAGCACAGTGTCCACGGTG |
| | | | | ACCTCTGCTGCAGTTGGTGGACAAGGACCCCC |
| | | | | TCGAACCTTCACCTGTCAGCTGACCTGGCACA |
| | | | | GAGACTCCGTAAGCTTCAGCCGTAGAAACGC |
| | | | | CTCTGGAACCGCCAGTGTGTTGCCGAGGCCGA |
| | | | | CTATCACGATGGAATTCACAGGCGATCATGCC |
| | | | | GTCTGTACTGCCGGCTGTGTGCCAGAAGGCGT |
| | | | | AACCTTCGCTTGGTTTCTCGGGGATGACTCAA |
| | | | | GTCCTGCAGAGAAAGTGGCTGTGGCCTCTCAG |
| | | | | ACGAGCTGCGGTCGACCAGGAACAGCTACCA |
| | | | | TTCGCAGCACTCTGCCCGTGTCCTACGAGCAG |
| | | | | ACGGAGTACATCTGCAGGCTGGCCGGCTATCC |
| | | | | CGATGGGATTCCAGTCCTGGAGCACCACTGAT |
| | | | | AA |
| 183 | EboZ | HSV-2 | Version | ATGGGAGTAACCGGAATTCTCCAGCTGCCAA |
| | | gC | 3 | GAGATCGATTCAAAAGAACATCATTTTTCCTT |
| | | | | TGGGTAATTATTCTGTTTCAGAGAACATTTTC |
| | | | | CATCCCTTCTCCTGGAAGAACCATCACAGTGG |
| | | | | GACCAAGAGGAAATGCAAGCAATGCAGCACC |
| | | | | TTCTGCTTCTCCAAGAAATGCTTCTGCTCCAA |
| | | | | GAACCACCCCAACCCCTCCTCAGCCAAGAAA |
| | | | | AGCAACCAAATCCAAAGCATCCACAGCAAAA |
| | | | | CCTGCACCTCCTCCAAAAACAGGACCTCCAAA |
| | | | | AACCTCCTCTGAACCTGTGAGATGCAACAGAC |
| | | | | ATGATCCTCTGGCAAGATATGGATCAAGAGTG |
| | | | | CAGATCAGATGCAGATTTCCAAATTCCACCAG |
| | | | | AACAGAATTCAGACTCCAGATCTGGAGATAT |
| | | | | GCAACAGCAACAGATGCAGAAATTGGAACAG |
| | | | | CACCATCTCTGGAAGAAGTGATGGTGAATGTG |
| | | | | TCTGCTCCTCCTGGAGGACAGCTGGTGTATGA |
| | | | | TTCTGCTCCAAACAGAACAGATCCTCATGTGA |
| | | | | TCTGGGCTGAAGGAGCTGGACCTGGAGCTTCT |
| | | | | CCAAGACTGTACTCTGTGGTGGGACCTCTGGG |
| | | | | AAGACAGAGACTGATCATTGAAGAACTGACC |
| | | | | CTGGAAACCCAGGGAATGTACTACTGGGTGT |
| | | | | GGGGAAGAACAGACAGACCTTCTGCTTATGG |
| | | | | AACCTGGGTGAGAGTGAGAGTGTTCAGACCT |
| | | | | CCTTCTCTGACCATCCACCCTCATGCTGTGCTG |
| | | | | GAAGGACAGCCTTTCAAAGCAACCTGCACAG |
| | | | | CAGCAACCTACTACCCTGGAAACAGAGCTGA |
| | | | | ATTTGTGTGGTTTGAAGATGGAAGAAGGGTGT |
| | | | | TTGATCCTGCTCAGATCCACACCCAGACCCAG |
| | | | | GAAAATCCTGATGGATTTTCCACAGTGTCCAC |
| | | | | AGTGACATCTGCTGCTGTGGGAGGACAGGGA |
| | | | | CCTCCAAGAACCTTCACCTGCCAGCTGACCTG |
| | | | | GCACAGAGATTCTGTGTCTTTTTCAAGAAGAA |
| | | | | ATGCTTCTGGAACAGCTTCTGTGCTGCCAAGA |
| | | | | CCAACCATCACCATGGAATTCACAGGAGATC |
| | | | | ATGCTGTGTGCACAGCTGGATGTGTGCCTGAA |
| | | | | GGAGTGACCTTTGCTTGGTTTCTGGGAGATGA |
| | | | | TTCTTCTCCAGCTGAAAAAGTGGCTGTGGCTT |
| | | | | CCCAGACCTCTTGTGGAAGACCTGGAACAGC |
| | | | | AACCATCAGATCCACCCTGCCTGTGTCTTATG |
| | | | | AACAGACAGAATACATTTGCAGACTGGCTGG |
| | | | | ATACCCTGATGGAATCCCTGTGCTGGAACACC |
| | | | | ACTGATAA |
| 184 | EboZ | HSV-2 | Version | ATGGGAGTTACCGGAATTCTGCAATTGCCCAG |
| | | gC | 4 | GGATCGGTTCAAGCGCACATCCTTCTTCCTGT |
| | | | | GGGTCATCATCCTCTTTCAGCGTACTTTCTCCA |
| | | | | TACCCTCTCCCGGACGAACTATCACTGTAGGC |
| | | | | CCAAGGGGCAACGCTAGTAATGCCGCTCCCA |
| | | | | GTGCTTCACCACGCAATGCGAGCGCACCCAG |
| | | | | AACTACACCCACACCTCCTCAGCCGAGGAAA |
| | | | | GCCACCAAGTCCAAAGCCAGCACCGCCAAAC |
| | | | | CCGCTCCTCCACCTAAAACAGGGCCTCCCAAG |
| | | | | ACCTCAAGCGAGCCCGTTAGATGCAATCGGC |
| | | | | ATGATCCACTGGCTCGTTACGGTTCTCGGGTC |
| | | | | CAGATACGCTGTAGGTTTCCTAACTCCACACG |
| | | | | AACCGAGTTCAGATTGCAGATCTGGAGATATG |
| | | | | CCACAGCCACTGACGCTGAGATTGGCACTGCA |
| | | | | CCTAGTCTGGAGGAGGTGATGGTGAACGTGA |
| | | | | GCGCCCCTCCTGGCGGTCAGCTTGTGTATGAC |
| | | | | TCAGCACCGAATCGCACAGATCCGCACGTCAT |
| | | | | ATGGGCCGAAGGTGCAGGGCCCGGTGCATCC |
| | | | | CCTCGGCTGTATTCCGTGGTCGGACCACTCGG |
| | | | | GCGCCAGAGGCTTATCATTGAGGAACTGACCC |
| | | | | TCGAAACCCAGGGTATGTACTACTGGGTATGG |
| | | | | GGCCGTACCGACCGGCCTAGCGCCTACGGAA |
| | | | | CTTGGGTGAGAGTTCGGGTGTTCAGACCGCCA |
| | | | | AGTCTTACAATTCACCCTCATGCCGTGCTCGA |
| | | | | AGGTCAGCCATTTAAGGCCACGTGTACTGCGG |
| | | | | CTACGTACTATCCCGGGAATCGGGCTGAATTC |
| | | | | GTGTGGTTTGAGGATGGCAGGAGAGTGTTCG |
| | | | | ACCCAGCCCAAATCCACACCCAAACACAGGA |
| | | | | GAACCCAGACGGGTTTTCCACCGTGTCAACGG |
| | | | | TCACATCTGCCGCCGTCGGAGGACAAGGGCC |
| | | | | ACCCAGAACCTTCACATGCCAGCTGACCTGGC |
| | | | | ATAGGGATAGCGTAAGCTTTAGCCGGCGAAA |
| | | | | CGCATCTGGAACGGCGAGCGTTCTGCCTCGAC |
| | | | | CAACAATCACCATGGAGTTCACCGGCGATCAC |
| | | | | GCAGTGTGCACTGCTGGGTGTGTACCCGAAGG |
| | | | | CGTGACATTTGCCTGGTTTCTGGGAGATGACT |
| | | | | CCTCACCCGCAGAAAAGGTCGCCGTTGCATCT |
| | | | | CAGACCAGTTGTGGCAGGCCCGGGACTGCTA |
| | | | | CCATCCGCAGCACTCTGCCGGTGTCTTACGAA |
| | | | | CAGACGGAGTACATTTGCCGCTTGGCGGGCTA |
| | | | | TCCAGACGGCATTCCAGTTCTGGAGCATCACT |
| | | | | GA |
| 89 | HSV-2 | HSV-2 | | ATGGGCCGCCTGACCTCCGGCGTGGGCACCGC |
| | gD | gD | | CGCCCTGCTGGTGGTGGCCGTGGGCCTGCGCG |
| | | | | TGGTGTGCGCCAAGTACGCCCTGGCCGACCCC |
| | | | | TCCCTGAAGATGGCCGACCCCAACCGCTTCCG |
| | | | | CGGCAAGAACCTGCCCGTGCTGGACCAGCTG |
| | | | | ACCGACCCCCCCGGCGTGAAGCGCGTGTACC |
| | | | | ACATCCAGCCCTCCCTGGAGGACCCCTTCCAG |
| | | | | CCCCCCTCCATCCCCATCACCGTGTACTACGC |
| | | | | CGTGCTGGAGCGCGCCTGCCGCTCCGTGCTGC |
| | | | | TGCACGCCCCCTCCGAGGCCCCCCAGATCGTG |
| | | | | CGCGGCGCCTCCGACGAGGCCCGCAAGCACA |
| | | | | CCTACAACCTGACCATCGCCTGGTACCGCATG |
| | | | | GGCGACAACTGCGCCATCCCCATCACCGTGAT |
| | | | | GGAGTACACCGAGTGCCCCTACAACAAGTCC |
| | | | | CTGGGCGTGTGCCCCATCCGCACCCAGCCCCG |
| | | | | CTGGTCCTACTACGACTCCTTCTCCGCCGTGTC |
| | | | | CGAGGACAACCTGGGCTTCCTGATGCACGCCC |
| | | | | CCGCCTTCGAGACCGCCGGCACCTACCTGCGC |
| | | | | CTGGTGAAGATCAACGACTGGACCGAGATCA |
| | | | | CCCAGTTCATCCTGGAGCACCGCGCCCGCGCC |
| | | | | TCCTGCAAGTACGCCCTGCCCCTGCGCATCCC |
| | | | | CCCCGCCGCCTGCCTGACCTCCAAGGCCTACC |
| | | | | AGCAGGGCGTGACCGTGGACTCCATCGGCAT |
| | | | | GCTGCCCCGCTTCATCCCCGAGAACCAGCGCA |
| | | | | CCGTGGCCCTGTACTCCCTGAAGATCGCCGGC |
| | | | | TGGCACGGCCCCAAGCCCCCCTACACCTCCAC |
| | | | | CCTGCTGCCCCCCGAGCTGTCCGACACCACCA |
| | | | | ACGCCACCCAGCCCGAGCTGGTGCCCGAGGA |
| | | | | CCCCGAGGACTCCGCCCTGCTGGAGGACCCCG |
| | | | | CCGGCACCGTGTCCTCCCAGATCCCCCCCAAC |
| | | | | TGGCACATCCCCTCCATCCAGGACGTGGCCCC |
| | | | | CCACCACTAA |
| 90 | HSV-2 | HSV-2 | Version | ATGGGCAGACTGACATCTGGCGTGGGAACAG |
| | gD | gD | 1 | CTGCTCTGCTGGTGGTTGCTGTGGGCCTGAGA |
| | | | | GTCGTGTGTGCCAAATACGCCCTGGCCGATCC |
| | | | | TAGCCTGAAGATGGCTGACCCCAACCGGTTCC |
| | | | | GGGGCAAGAATCTGCCTGTTCTGGACCAGCTG |
| | | | | ACCGATCCTCCTGGCGTGAAACGGGTGTACCA |
| | | | | CATCCAGCCAAGCCTGGAAGATCCCTTCCAGC |
| | | | | CTCCTAGCATCCCCATCACCGTGTACTACGCC |
| | | | | GTGCTGGAAAGGGCCTGTAGAAGCGTGCTGC |
| | | | | TGCACGCCCCATCTGAAGCCCCTCAAATCGTC |
| | | | | AGAGGCGCTTCCGACGAGGCCAGAAAGCACA |
| | | | | CCTACAACCTGACAATCGCCTGGTACAGAATG |
| | | | | GGCGACAACTGCGCCATTCCTATCACCGTGAT |
| | | | | GGAGTACACCGAGTGTCCCTACAACAAGAGC |
| | | | | CTGGGCGTGTGCCCCATCAGAACACAGCCTAG |
| | | | | ATGGTCCTACTACGACAGCTTCAGCGCCGTGT |
| | | | | CCGAGGACAATCTGGGCTTCCTGATGCATGCC |
| | | | | CCTGCCTTTGAGACAGCCGGCACCTATCTGCG |
| | | | | GCTGGTCAAGATCAACGACTGGACCGAGATC |
| | | | | ACCCAGTTCATCCTGGAACACAGAGCCAGAG |
| | | | | CCAGCTGCAAATACGCTCTGCCCCTGAGAATT |
| | | | | CCTCCTGCCGCCTGTCTGACAAGCAAGGCCTA |
| | | | | TCAGCAGGGCGTGACCGTGGATAGCATCGGC |
| | | | | ATGCTGCCCAGATTCATCCCCGAGAACCAGAG |
| | | | | AACAGTGGCCCTGTACTCCCTGAAGATCGCCG |
| | | | | GATGGCACGGACCCAAGCCTCCATACACAAG |
| | | | | CACACTGCTGCCTCCAGAGCTGAGCGACACCA |
| | | | | CCAATGCCACACAGCCTGAACTGGTGCCTGAG |
| | | | | GACCCAGAGGATTCTGCCCTGCTTGAAGATCC |
| | | | | TGCCGGCACCGTGTCTAGCCAGATTCCTCCTA |
| | | | | ACTGGCACATCCCCAGCATCCAGGATGTGGCC |
| | | | | CCTCATCATTGA |
| 91 | HSV-2 | HSV-2 | Version | ATGGGGAGACTCACATCAGGCGTAGGAACCG |
| | gD | gD | 2 | CTGCCCTGTTGGTCGTGGCCGTTGGTCTGAGA |
| | | | | GTTGTGTGTGCCAAATATGCTCTCGCTGATCC |
| | | | | GAGCCTCAAGATGGCAGATCCCAACCGATTTC |
| | | | | GGGGAAAGAATCTGCCAGTACTGGACCAGCT |
| | | | | GACGGACCCACCTGGCGTCAAACGCGTCTACC |
| | | | | ACATACAGCCTAGTCTTGAGGACCCTTTTCAG |
| | | | | CCACCGTCTATCCCCATTACCGTGTACTATGC |
| | | | | CGTGCTGGAACGCGCGTGTAGGTCAGTTCTGC |
| | | | | TGCATGCCCCATCCGAAGCCCCCCAGATCGTC |
| | | | | AGAGGAGCTTCTGATGAAGCACGCAAACACA |
| | | | | CCTACAACCTCACAATAGCGTGGTATCGAATG |
| | | | | GGCGATAACTGCGCAATTCCCATCACAGTCAT |
| | | | | GGAGTACACGGAGTGCCCCTACAACAAGAGC |
| | | | | CTCGGTGTTTGCCCTATCAGGACACAACCCAG |
| | | | | GTGGAGCTATTACGACAGTTTCAGCGCCGTGT |
| | | | | CTGAGGACAATCTGGGGTTTCTGATGCACGCA |
| | | | | CCCGCCTTCGAGACTGCCGGCACCTACTTGCG |
| | | | | GCTGGTGAAGATCAACGACTGGACTGAGATC |
| | | | | ACCCAGTTCATCCTGGAACATAGGGCCAGAG |
| | | | | CCAGCTGCAAGTATGCTCTTCCCCTGCGGATT |
| | | | | CCGCCTGCAGCATGTCTGACCTCAAAAGCCTA |
| | | | | CCAGCAAGGGGTGACTGTGGACAGCATTGGC |
| | | | | ATGCTGCCTCGTTTCATTCCCGAGAATCAACG |
| | | | | GACAGTGGCTCTGTATTCCCTGAAGATCGCAG |
| | | | | GATGGCATGGGCCCAAACCACCTTATACCTCT |
| | | | | ACGTTGCTTCCACCAGAACTCAGTGACACCAC |
| | | | | TAATGCGACACAGCCAGAACTTGTGCCTGAG |
| | | | | GATCCTGAAGATAGCGCTCTGTTGGAGGATCC |
| | | | | AGCCGGTACTGTGTCCTCCCAGATACCACCCA |
| | | | | ATTGGCACATTCCTTCCATTCAGGACGTAGCT |
| | | | | CCGCATCACTGA |
| 92 | HSV-2 | HSV-2 | Version | ATGGGGAGACTCACATCAGGCGTAGGAACCG |
| | gD | gD | 2.1 | CTGCCCTGTTGGTCGTGGCCGTTGGTCTGAGA |
| | | | | GTTGTGTGTGCCAAATATGCTCTCGCTGATCC |
| | | | | GAGCCTCAAGATGGCAGATCCCAACCGATTTC |
| | | | | GGGGAAAGAATCTGCCAGTACTGGACCAGCT |
| | | | | GACGGACCCACCTGGCGTCAAACGCGTCTACC |
| | | | | ACATACAGCCTAGTCTTGAGGACCCTTTTCAG |
| | | | | CCACCGTCTATCCCCATTACCGTGTACTATGC |
| | | | | CGTGCTGGAACGCGCGTGTAGGTCAGTTCTGC |
| | | | | TGCATGCCCCATCCGAAGCCCCCCAGATCGTC |
| | | | | AGAGGAGCTTCTGATGAAGCACGCAAACACA |
| | | | | CCTACAACCTCACAATAGCGTGGTATCGAATG |
| | | | | GGCGATAACTGCGCAATTCCCATCACAGTCAT |
| | | | | GGAGTACACGGAGTGCCCCTACAACAAGAGC |
| | | | | CTCGGTGTTTGCCCTATCAGGACACAACCCAG |
| | | | | GTGGAGCTATTACGACAGTTTCAGCGCCGTGT |
| | | | | CTGAGGACAATCTGGGGTTTCTGATGCACGCA |
| | | | | CCCGCCTTCGAGACTGCCGGCACCTACTTGCG |
| | | | | GCTGGTGAAGATCAACGACTGGACTGAGATC |
| | | | | ACCCAGTTCATCCTGGAACATAGGGCCAGAG |
| | | | | CCAGCTGCAAGTATGCCCTTCCCCTGCGGATT |
| | | | | CCGCCTGCAGCATGTCTGACCTCAAAAGCCTA |
| | | | | CCAGCAAGGGGTGACTGTGGACAGCATTGGC |
| | | | | ATGCTGCCTCGTTTCATTCCCGAGAATCAACG |
| | | | | GACAGTGGCTCTGTATTCCCTGAAGATCGCAG |
| | | | | GATGGCATGGGCCCAAACCACCTTATACCTCT |
| | | | | ACGTTGCTTCCACCAGAACTCAGTGACACCAC |
| | | | | TAATGCGACACAGCCAGAACTTGTGCCTGAG |
| | | | | GATCCTGAAGATAGCGCTCTGTTGGAGGATCC |
| | | | | AGCCGGTACTGTGTCCTCCCAGATACCACCCA |
| | | | | ATTGGCACATTCCTTCCATTCAGGACGTAGCT |
| | | | | CCGCATCACTGA |
| 346 | HSV-2 | HSV-2 | Version | ATGGGGAGACTCACATCAGGCGTAGGAACCG |
| | gD | gD | 2.2 | CTGCCCTGTTGGTCGTGGCCGTTGGTCTGAGA |
| | | | | GTTGTGTGTGCCAAATATGCTCTCGCTGATCC |
| | | | | GAGCCTCAAGATGGCAGATCCCAACCGATTTC |
| | | | | GGGGAAAGAATCTGCCAGTACTGGACCAGCT |
| | | | | GACGGACCCACCTGGCGTCAAACGCGTCTACC |
| | | | | ACATACAGCCTAGTCTTGAGGACCCTTTTCAG |
| | | | | CCACCGTCTATCCCCATTACCGTGTACTATGC |
| | | | | CGTGCTGGAACGCGCGTGTAGGTCAGTTCTGC |
| | | | | TGCATGCCCCATCCGAAGCCCCCCAGATCGTC |
| | | | | AGAGGAGCTTCTGATGAAGCACGCAAACACA |
| | | | | CCTACAACCTCACAATAGCGTGGTATCGAATG |
| | | | | GGCGATAACTGCGCAATTCCCATCACAGTCAT |
| | | | | GGAGTACACGGAGTGCCCCTACAACAAGAGC |
| | | | | CTCGGTGTTTGCCCTATCAGGACACAACCCAG |
| | | | | GTGGAGCTATTACGACAGTTTCAGCGCCGTGT |
| | | | | CTGAGGACAATCTGGGGTTTCTGATGCACGCA |
| | | | | CCCGCCTTCGAGACTGCCGGCACCTACTTGCG |
| | | | | GCTGGTGAAGATCAACGACTGGACTGAGATC |
| | | | | ACCCAGTTCATCCTGGAACATAGGGCCAGAG |
| | | | | CCAGCTGCAAGTATGCCCTTCCCCTGCGGATT |
| | | | | CCGCCTGCAGCATGTCTGACCTCAAAAGCCTA |
| | | | | CCAGCAAGGGGTGACTGTGGACAGCATTGGC |
| | | | | ATGCTGCCTCGTTTCATTCCCGAGAATCAACG |
| | | | | GACAGTGGCTCTGTATTCCCTGAAGATCGCAG |
| | | | | GATGGCATGGGCCCAAACCACCTTATACCTCT |
| | | | | ACGTTGCTTCCACCAGAACTCAGTGACACCAC |
| | | | | TAATGCGACACAGCCAGAACTTGTGCCTGAG |
| | | | | GATCCTGAAGATAGCGCTCTGTTGGAGGATCC |
| | | | | AGCCGGTACTGTGTCCTCCCAGATACCACCCA |
| | | | | ATTGGCACATTCCTTCCATTCAGGACGTAGCT |
| | | | | CCGCATCACTGATAA |
| 93 | HSV-2 | HSV-2 | Version | ATGGGCAGACTGACCTCCGGCGTGGGCACCG |
| | gD | gD | 3 | CCGCCCTGCTGGTGGTGGCCGTGGGCCTGAGA |
| | | | | GTGGTGTGCGCCAAATACGCCCTGGCCGATCC |
| | | | | CTCCCTGAAAATGGCCGATCCCAACAGGTTCA |
| | | | | GAGGCAAAAACCTGCCCGTGCTGGATCAGCT |
| | | | | GACCGATCCCCCTGGCGTGAAAAGAGTGTAC |
| | | | | CACATCCAGCCCTCCCTGGAAGATCCCTTCCA |
| | | | | GCCCCCTTCCATCCCCATCACCGTGTACTACG |
| | | | | CCGTGCTGGAAAGAGCTTGCAGATCCGTGCTG |
| | | | | CTGCACGCCCCCTCCGAAGCCCCTCAGATCGT |
| | | | | GAGAGGCGCCTCCGATGAAGCCAGAAAACAC |
| | | | | ACCTACAACCTGACCATCGCCTGGTACAGAAT |
| | | | | GGGCGATAACTGCGCCATCCCCATCACCGTGA |
| | | | | TGGAATACACCGAATGCCCCTACAACAAATCC |
| | | | | CTGGGCGTGTGCCCCATCAGAACCCAGCCCAG |
| | | | | ATGGTCCTACTACGATTCCTTCTCCGCCGTGTC |
| | | | | CGAAGATAACCTGGGCTTCCTGATGCACGCCC |
| | | | | CCGCCTTCGAAACCGCCGGCACCTACCTGAGA |
| | | | | CTGGTGAAAATCAACGATTGGACCGAAATCA |
| | | | | CCCAGTTCATCCTGGAACACAGAGCCAGAGC |
| | | | | CTCCTGCAAATACGCCCTGCCCCTGAGAATCC |
| | | | | CTCCCGCCGCCTGCCTGACCTCCAAAGCCTAC |
| | | | | CAGCAGGGCGTGACCGTGGATTCCATCGGCAT |
| | | | | GCTGCCCAGATTCATCCCCGAAAACCAGAGA |
| | | | | ACCGTGGCCCTGTACTCCCTGAAAATCGCCGG |
| | | | | CTGGCACGGCCCCAAACCCCCTTACACCTCCA |
| | | | | CCCTGCTGCCCCCTGAACTGTCCGATACCACC |
| | | | | AACGCCACCCAGCCCGAACTGGTGCCCGAAG |
| | | | | ATCCCGAAGATTCCGCCCTGCTGGAAGATCCC |
| | | | | GCCGGCACCGTGTCCTCCCAGATCCCTCCCAA |
| | | | | CTGGCACATCCCCTCCATCCAGGATGTGGCCC |
| | | | | CTCACCACTGA |
| 94 | IL2 | HSV-2 | | ATGCGCATGCAGCTGCTGCTGCTGATCGCCCT |
| | | gE | | GTCCCTGGCCCTGGTGACCAACTCCCGCACCT |
| | | | | CCTGGAAGCGCGTGACCTCCGGCGAGGACGT |
| | | | | GGTGCTGCTGCCCGCCCCCGCCGGCCCCGAGG |
| | | | | AGCGCACCCGCGCCCACAAGCTGCTGTGGGC |
| | | | | CGCCGAGCCCCTGGACGCCTGCGGCCCCCTGC |
| | | | | GCCCCTCCTGGGTGGCCCTGTGGCCCCCCCGC |
| | | | | CGCGTGCTGGAGACCGTGGTGGACGCCGCCT |
| | | | | GCATGCGCGCCCCCGAGCCCCTGGCCATCGCC |
| | | | | TACTCCCCCCCCTTCCCCGCCGGCGACGAGGG |
| | | | | CCTGTACTCCGAGCTGGCCTGGCGCGACCGCG |
| | | | | TGGCCGTGGTGAACGAGTCCCTGGTGATCTAC |
| | | | | GGCGCCCTGGAGACCGACTCCGGCCTGTACAC |
| | | | | CCTGTCCGTGGTGGGCCTGTCCGACGAGGCCC |
| | | | | GCCAGGTGGCCTCCGTGGTGCTGGTGGTGGAG |
| | | | | CCCGCCCCCGTGCCCACCCCCACCCCCGACGA |
| | | | | CTACGACGAGGAGGACGACGCCGGCGTGTCC |
| | | | | GAGCGCACCCCCGTGTCCGTGCCCCCCCCCAC |
| | | | | CCCCCCCCGCCGCCCCCCCGTGGCCCCCCCCA |
| | | | | CCCACCCCCGCGTGATCCCCGAGGTGTCCCAC |
| | | | | GTGCGCGGCGTGACCGTGCACATGGAGACCC |
| | | | | CCGAGGCCATCCTGTTCGCCCCCGGCGAGACC |
| | | | | TTCGGCACCAACGTGTCCATCCACGCCATCGC |
| | | | | CCACGACGACGGCCCCTACGCCATGGACGTG |
| | | | | GTGTGGATGCGCTTCGACGTGCCCTCCTCCTG |
| | | | | CGCCGAGATGCGCATCTACGAGGCCTGCCTGT |
| | | | | ACCACCCCCAGCTGCCCGAGTGCCTGTCCCCC |
| | | | | GCCGACGCCCCCTGCGCCGTGTCCTCCTGGGC |
| | | | | CTACCGCCTGGCCGTGCGCTCCTACGCCGGCT |
| | | | | GCTCCCGCACCACCCCCCCCCCCCGCTGCTTC |
| | | | | GCCGAGGCCCGCATGGAGCCCGTGCCCGGCC |
| | | | | TGGCCTGGCTGGCCTCCACCGTGAACCTGGAG |
| | | | | TTCCAGCACGCCTCCCCCCAGCACGCCGGCCT |
| | | | | GTACCTGTGCGTGGTGTACGTGGACGACCACA |
| | | | | TCCACGCCTGGGGCCACATGACCATCTCCACC |
| | | | | GCCGCCCAGTACCGCAACGCCGTGGTGGAGC |
| | | | | AGCACCTGCCCCAGCGCCAGCCCGAGCCCGT |
| | | | | GGAGCCCACCCGCCCCCACGTGCGCGCCTAA |
| 95 | IL2 | HSV-2 | Version | ATGAGAATGCAGCTGCTGCTCCTGATCGCCCT |
| | | gE | 1 | GTCTCTGGCCCTGGTCACCAATAGCAGAACCA |
| | | | | GCTGGAAAAGAGTGACCAGCGGCGAGGATGT |
| | | | | GGTGCTGCTTCCTGCTCCTGCTGGCCCCGAGG |
| | | | | AAAGAACAAGAGCCCACAAACTGCTGTGGGC |
| | | | | CGCTGAGCCTCTTGATGCCTGTGGACCTCTCA |
| | | | | GACCTAGCTGGGTTGCACTGTGGCCACCTCGG |
| | | | | AGAGTGCTGGAAACAGTGGTGGATGCCGCCT |
| | | | | GCATGAGAGCCCCTGAACCTCTGGCCATTGCC |
| | | | | TACTCTCCACCATTTCCAGCCGGCGACGAGGG |
| | | | | CCTGTATTCTGAGCTTGCTTGGAGAGACAGAG |
| | | | | TGGCCGTGGTCAACGAGAGCCTGGTTATCTAT |
| | | | | GGCGCCCTGGAAACCGACAGCGGCCTGTACA |
| | | | | CACTGTCTGTCGTGGGCCTGTCTGACGAGGCT |
| | | | | AGACAGGTGGCATCTGTGGTCCTGGTGGTGGA |
| | | | | ACCTGCTCCAGTGCCTACACCTACACCTGACG |
| | | | | ACTACGACGAGGAAGATGACGCTGGCGTCAG |
| | | | | CGAGAGAACCCCTGTTTCTGTGCCTCCTCCTA |
| | | | | CGCCTCCTCGTAGACCTCCTGTTGCTCCTCCA |
| | | | | ACACACCCCAGAGTGATCCCTGAAGTGTCTCA |
| | | | | CGTGCGGGGCGTGACCGTGCACATGGAAACA |
| | | | | CCTGAGGCCATCCTGTTCGCCCCTGGCGAGAC |
| | | | | ATTTGGCACCAACGTGTCCATCCACGCTATCG |
| | | | | CCCACGACGATGGCCCTTACGCCATGGATGTC |
| | | | | GTGTGGATGAGATTCGACGTGCCCAGCAGCTG |
| | | | | TGCCGAGATGAGAATCTATGAGGCCTGCCTGT |
| | | | | ATCACCCTCAGCTGCCCGAATGTCTGAGCCCT |
| | | | | GCTGATGCCCCTTGTGCCGTTAGCAGCTGGGC |
| | | | | CTATAGACTGGCCGTGCGGTCTTATGCCGGCT |
| | | | | GCTCTAGAACAACCCCTCCTCCTCGGTGTTTC |
| | | | | GCCGAGGCCAGAATGGAACCTGTTCCTGGACT |
| | | | | GGCCTGGCTGGCCTCCACAGTGAACCTGGAAT |
| | | | | TTCAGCACGCCTCTCCACAGCACGCCGGCCTG |
| | | | | TATCTGTGTGTGGTGTACGTGGACGATCACAT |
| | | | | CCACGCCTGGGGCCACATGACCATCTCTACAG |
| | | | | CCGCTCAGTACCGGAACGCCGTGGTTGAACA |
| | | | | GCATCTGCCTCAGAGACAGCCCGAGCCTGTGG |
| | | | | AACCTACAAGACCTCATGTTCGGGCCTGA |
| 96 | IL2 | HSV-2 | Version | ATGCGAATGCAGCTTCTGCTGCTCATTGCCTT |
| | | gE | 2 | GTCCCTTGCCTTGGTGACCAACTCACGCACCT |
| | | | | CTTGGAAACGCGTTACTTCCGGGGAGGACGTT |
| | | | | GTCCTCCTTCCAGCACCCGCAGGACCTGAAGA |
| | | | | GAGGACTAGGGCCCACAAGCTGCTGTGGGCC |
| | | | | GCTGAACCTCTGGATGCCTGTGGTCCTCTGAG |
| | | | | ACCTAGCTGGGTCGCCCTTTGGCCACCTAGAC |
| | | | | GCGTTCTGGAGACGGTCGTGGATGCCGCGTGC |
| | | | | ATGCGTGCACCCGAACCTCTGGCCATCGCCTA |
| | | | | TAGTCCCCCTTTTCCCGCTGGCGACGAGGGGC |
| | | | | TTTACTCCGAACTGGCCTGGCGGGATAGGGTG |
| | | | | GCGGTGGTGAACGAGAGCCTCGTCATCTACG |
| | | | | GTGCTCTGGAAACCGACTCAGGACTGTATACG |
| | | | | CTCAGCGTTGTTGGCCTCTCCGATGAGGCTCG |
| | | | | ACAGGTTGCCTCCGTAGTGCTGGTCGTAGAAC |
| | | | | CAGCCCCCGTACCAACACCCACACCCGACGA |
| | | | | CTACGACGAAGAGGACGACGCTGGAGTTAGC |
| | | | | GAAAGAACACCGGTGAGTGTGCCACCTCCCA |
| | | | | CACCGCCAAGGAGACCCCCAGTAGCACCTCC |
| | | | | AACCCATCCGAGAGTGATTCCCGAGGTCAGCC |
| | | | | ATGTGCGCGGCGTAACTGTGCACATGGAGAC |
| | | | | GCCCGAAGCGATACTGTTTGCCCCTGGAGAGA |
| | | | | CATTCGGCACCAATGTGTCCATACACGCAATT |
| | | | | GCGCACGATGATGGCCCATACGCTATGGACGT |
| | | | | CGTCTGGATGAGGTTCGATGTGCCTTCTTCTT |
| | | | | GCGCCGAGATGAGGATCTACGAGGCATGCCT |
| | | | | GTATCACCCCCAATTGCCGGAGTGTCTGTCTC |
| | | | | CCGCAGATGCACCGTGTGCAGTGAGTAGCTG |
| | | | | GGCTTATCGGTTGGCTGTCCGGAGTTATGCTG |
| | | | | GGTGTTCACGGACCACCCCACCTCCACGTTGC |
| | | | | TTTGCTGAAGCCAGAATGGAACCCGTGCCTGG |
| | | | | TCTGGCTTGGCTGGCATCAACTGTCAACCTGG |
| | | | | AGTTCCAGCATGCCTCTCCACAGCACGCAGGC |
| | | | | CTGTATCTCTGCGTGGTGTACGTTGACGATCA |
| | | | | CATCCATGCGTGGGGGCATATGACCATCAGCA |
| | | | | CAGCTGCCCAGTACCGCAATGCCGTCGTGGAG |
| | | | | CAGCACCTCCCCCAACGGCAGCCAGAACCAG |
| | | | | TGGAGCCCACTCGGCCTCATGTGCGAGCCTGA |
| 97 | IL2 | HSV-2 | Version | ATGAGAATGCAGCTGCTGCTGCTGATCGCCCT |
| | | gE | 3 | GTCCCTGGCCCTGGTGACCAACTCCAGAACCT |
| | | | | CCTGGAAAAGAGTGACCTCCGGCGAAGATGT |
| | | | | GGTGCTGCTGCCCGCCCCCGCCGGCCCCGAAG |
| | | | | AAAGAACCAGAGCCCACAAACTGCTGTGGGC |
| | | | | CGCCGAACCCCTGGATGCCTGCGGCCCCCTCA |
| | | | | GACCCTCCTGGGTGGCCCTGTGGCCCCCTAGA |
| | | | | AGGGTGCTGGAAACCGTGGTGGATGCCGCCT |
| | | | | GCATGAGAGCCCCCGAACCCCTGGCCATCGCC |
| | | | | TACTCCCCTCCCTTCCCCGCCGGCGATGAAGG |
| | | | | CCTGTACTCCGAACTGGCCTGGAGAGATAGA |
| | | | | GTGGCCGTGGTGAACGAATCCCTGGTGATCTA |
| | | | | CGGCGCCCTGGAAACCGATTCCGGCCTGTACA |
| | | | | CCCTGTCCGTGGTGGGCCTGTCCGATGAAGCC |
| | | | | AGACAGGTGGCCTCCGTGGTGCTGGTGGTGG |
| | | | | AACCCGCCCCCGTGCCCACCCCCACCCCCGAT |
| | | | | GATTACGATGAAGAAGATGATGCCGGCGTGT |
| | | | | CCGAAAGAACCCCCGTGTCCGTGCCCCCTCCC |
| | | | | ACCCCTCCCCGCAGACCCCCTGTGGCCCCTCC |
| | | | | CACCCACCCCAGAGTGATCCCCGAAGTGTCCC |
| | | | | ACGTGAGAGGCGTGACCGTGCACATGGAAAC |
| | | | | CCCCGAAGCCATCCTGTTCGCCCCCGGCGAAA |
| | | | | CCTTCGGCACCAACGTGTCCATCCACGCCATC |
| | | | | GCCCACGATGATGGCCCCTACGCCATGGATGT |
| | | | | GGTGTGGATGAGATTCGATGTGCCCTCCTCCT |
| | | | | GCGCCGAAATGAGAATCTACGAAGCCTGCCT |
| | | | | GTACCACCCCCAGCTGCCCGAATGCCTGTCCC |
| | | | | CCGCCGATGCCCCCTGCGCCGTGTCCTCCTGG |
| | | | | GCCTACAGACTGGCCGTGAGATCCTACGCCGG |
| | | | | CTGCTCCAGAACCACCCCTCCCCCTAGATGCT |
| | | | | TCGCCGAAGCCAGAATGGAACCCGTGCCCGG |
| | | | | CCTGGCCTGGCTGGCCTCCACCGTGAACCTGG |
| | | | | AATTCCAGCACGCCAGCCCCCAGCACGCCGG |
| | | | | CCTGTACCTGTGCGTGGTGTACGTGGATGATC |
| | | | | ACATCCACGCCTGGGGCCACATGACCATCTCC |
| | | | | ACCGCCGCCCAGTACAGAAACGCCGTGGTGG |
| | | | | AACAGCACCTGCCCCAGAGACAGCCCGAACC |
| | | | | CGTGGAACCCACCAGACCCCACGTGAGAGCC |
| | | | | TGA |
| 98 | HSV-2 | HSV-2 | | ATGGGCCGCCTGACCTCCGGCGTGGGCACCGC |
| | gD - | gE | | CGCCCTGCTGGTGGTGGCCGTGGGCCTGCGCG |
| | KYA | | | TGGTGTGCGCCAAGTACGCCCGCACCTCCTGG |
| | | | | AAGCGCGTGACCTCCGGCGAGGACGTGGTGC |
| | | | | TGCTGCCCGCCCCCGCCGGCCCCGAGGAGCGC |
| | | | | ACCCGCGCCCACAAGCTGCTGTGGGCCGCCG |
| | | | | AGCCCCTGGACGCCTGCGGCCCCCTGCGCCCC |
| | | | | TCCTGGGTGGCCCTGTGGCCCCCCCGCCGCGT |
| | | | | GCTGGAGACCGTGGTGGACGCCGCCTGCATG |
| | | | | CGCGCCCCCGAGCCCCTGGCCATCGCCTACTC |
| | | | | CCCCCCCTTCCCCGCCGGCGACGAGGGCCTGT |
| | | | | ACTCCGAGCTGGCCTGGCGCGACCGCGTGGCC |
| | | | | GTGGTGAACGAGTCCCTGGTGATCTACGGCGC |
| | | | | CCTGGAGACCGACTCCGGCCTGTACACCCTGT |
| | | | | CCGTGGTGGGCCTGTCCGACGAGGCCCGCCA |
| | | | | GGTGGCCTCCGTGGTGCTGGTGGTGGAGCCCG |
| | | | | CCCCCGTGCCCACCCCCACCCCCGACGACTAC |
| | | | | GACGAGGAGGACGACGCCGGCGTGTCCGAGC |
| | | | | GCACCCCCGTGTCCGTGCCCCCCCCCACCCCC |
| | | | | CCCCGCCGCCCCCCCGTGGCCCCCCCCACCCA |
| | | | | CCCCCGCGTGATCCCCGAGGTGTCCCACGTGC |
| | | | | GCGGCGTGACCGTGCACATGGAGACCCCCGA |
| | | | | GGCCATCCTGTTCGCCCCCGGCGAGACCTTCG |
| | | | | GCACCAACGTGTCCATCCACGCCATCGCCCAC |
| | | | | GACGACGGCCCCTACGCCATGGACGTGGTGT |
| | | | | GGATGCGCTTCGACGTGCCCTCCTCCTGCGCC |
| | | | | GAGATGCGCATCTACGAGGCCTGCCTGTACCA |
| | | | | CCCCCAGCTGCCCGAGTGCCTGTCCCCCGCCG |
| | | | | ACGCCCCCTGCGCCGTGTCCTCCTGGGCCTAC |
| | | | | CGCCTGGCCGTGCGCTCCTACGCCGGCTGCTC |
| | | | | CCGCACCACCCCCCCCCCCCGCTGCTTCGCCG |
| | | | | AGGCCCGCATGGAGCCCGTGCCCGGCCTGGC |
| | | | | CTGGCTGGCCTCCACCGTGAACCTGGAGTTCC |
| | | | | AGCACGCCTCCCCCCAGCACGCCGGCCTGTAC |
| | | | | CTGTGCGTGGTGTACGTGGACGACCACATCCA |
| | | | | CGCCTGGGGCCACATGACCATCTCCACCGCCG |
| | | | | CCCAGTACCGCAACGCCGTGGTGGAGCAGCA |
| | | | | CCTGCCCCAGCGCCAGCCCGAGCCCGTGGAG |
| | | | | CCCACCCGCCCCCACGTGCGCGCCTAA |
| 99 | HSV-2 | HSV-2 | Version | ATGGGCAGACTGACATCTGGCGTGGGAACAG |
| | gD - | gE | 1 | CTGCTCTGCTGGTGGTTGCTGTGGGCCTGAGA |
| | KYA | | | GTCGTGTGTGCCAAATACGCCAGAACCAGCTG |
| | | | | GAAAAGAGTGACCAGCGGCGAGGATGTGGTG |
| | | | | CTGCTTCCTGCTCCTGCTGGCCCCGAGGAAAG |
| | | | | AACAAGAGCCCACAAACTGCTGTGGGCCGCT |
| | | | | GAGCCTCTTGATGCCTGTGGACCTCTCAGACC |
| | | | | TAGCTGGGTTGCACTGTGGCCACCTCGGAGAG |
| | | | | TGCTGGAAACAGTGGTGGATGCCGCCTGCATG |
| | | | | AGAGCCCCTGAACCTCTGGCCATTGCCTACTC |
| | | | | TCCACCATTTCCAGCCGGCGACGAGGGCCTGT |
| | | | | ATTCTGAGCTTGCTTGGAGAGACAGAGTGGCC |
| | | | | GTGGTCAACGAGAGCCTGGTTATCTATGGCGC |
| | | | | CCTGGAAACCGACAGCGGCCTGTACACACTGT |
| | | | | CTGTCGTGGGCCTGTCTGACGAGGCTAGACAG |
| | | | | GTGGCATCTGTGGTCCTGGTGGTGGAACCTGC |
| | | | | TCCAGTGCCTACACCTACACCTGACGACTACG |
| | | | | ACGAGGAAGATGACGCTGGCGTCAGCGAGAG |
| | | | | AACCCCTGTTTCTGTGCCTCCTCCTACGCCTCC |
| | | | | TCGTAGACCTCCTGTTGCTCCTCCAACACACC |
| | | | | CCAGAGTGATCCCTGAAGTGTCTCACGTGCGG |
| | | | | GGCGTGACCGTGCACATGGAAACACCTGAGG |
| | | | | CCATCCTGTTCGCCCCTGGCGAGACATTTGGC |
| | | | | ACCAACGTGTCCATCCACGCTATCGCCCACGA |
| | | | | CGATGGCCCTTACGCCATGGATGTCGTGTGGA |
| | | | | TGAGATTCGACGTGCCCAGCAGCTGTGCCGAG |
| | | | | ATGAGAATCTATGAGGCCTGCCTGTATCACCC |
| | | | | TCAGCTGCCCGAATGTCTGAGCCCTGCTGATG |
| | | | | CCCCTTGTGCCGTTAGCAGCTGGGCCTATAGA |
| | | | | CTGGCCGTGCGGTCTTATGCCGGCTGCTCTAG |
| | | | | AACAACCCCTCCTCCTCGGTGTTTCGCCGAGG |
| | | | | CCAGAATGGAACCTGTTCCTGGACTGGCCTGG |
| | | | | CTGGCCTCCACAGTGAACCTGGAATTTCAGCA |
| | | | | CGCCTCTCCACAGCACGCCGGCCTGTATCTGT |
| | | | | GTGTGGTGTACGTGGACGATCACATCCACGCC |
| | | | | TGGGGCCACATGACCATCTCTACAGCCGCTCA |
| | | | | GTACCGGAACGCCGTGGTTGAACAGCATCTGC |
| | | | | CTCAGAGACAGCCCGAGCCTGTGGAACCTAC |
| | | | | AAGACCTCATGTTCGGGCCTGA |
| 100 | HSV-2 | HSV-2 | Version | ATGGGGAGACTCACATCAGGCGTAGGAACCG |
| | gD - | gE | 2 | CTGCCCTGTTGGTCGTGGCCGTTGGTCTGAGA |
| | KYA | | | GTTGTGTGTGCCAAATATGCTCGCACCTCTTG |
| | | | | GAAACGCGTTACTTCCGGGGAGGACGTTGTCC |
| | | | | TCCTTCCAGCACCCGCAGGACCTGAGGAAAG |
| | | | | GACTAGGGCCCACAAGCTGCTGTGGGCCGCT |
| | | | | GAACCTCTGGATGCCTGTGGTCCTCTGAGACC |
| | | | | TAGCTGGGTCGCCCTTTGGCCACCTAGACGCG |
| | | | | TTCTGGAGACGGTCGTGGATGCCGCGTGCATG |
| | | | | CGTGCACCCGAACCTCTGGCCATCGCCTATAG |
| | | | | TCCCCCTTTTCCCGCTGGCGACGAGGGGCTTT |
| | | | | ACTCCGAACTGGCCTGGCGGGATAGGGTGGC |
| | | | | GGTGGTGAACGAGAGCCTCGTCATCTACGGTG |
| | | | | CTCTGGAAACCGACTCAGGACTGTATACGCTC |
| | | | | AGCGTTGTTGGCCTCTCCGATGAGGCTCGACA |
| | | | | GGTTGCCTCCGTAGTGCTGGTCGTAGAACCAG |
| | | | | CCCCCGTACCAACACCCACACCCGACGACTAC |
| | | | | GACGAGGAGGACGACGCTGGAGTTAGCGAAA |
| | | | | GAACACCGGTGAGTGTGCCACCTCCCACACCG |
| | | | | CCAAGGAGACCCCCAGTAGCACCTCCAACCC |
| | | | | ATCCGAGAGTGATTCCCGAGGTCAGCCATGTG |
| | | | | CGCGGCGTAACTGTGCACATGGAGACGCCCG |
| | | | | AAGCGATACTGTTTGCCCCTGGAGAGACATTC |
| | | | | GGCACCAATGTGTCCATACACGCAATTGCGCA |
| | | | | CGATGATGGCCCATACGCTATGGACGTCGTCT |
| | | | | GGATGAGGTTCGATGTGCCTTCTTCTTGCGCC |
| | | | | GAGATGAGGATCTACGAGGCATGCCTGTATC |
| | | | | ACCCCCAATTGCCGGAGTGTCTGTCTCCCGCA |
| | | | | GATGCACCGTGTGCAGTGAGTAGCTGGGCTTA |
| | | | | TCGGTTGGCTGTCCGGAGTTATGCTGGGTGTT |
| | | | | CACGGACCACCCCACCTCCACGTTGCTTTGCT |
| | | | | GAAGCCAGAATGGAACCCGTGCCTGGTCTGG |
| | | | | CTTGGCTGGCATCAACTGTCAACCTGGAGTTC |
| | | | | CAGCATGCCTCTCCACAGCACGCAGGCCTGTA |
| | | | | TCTCTGCGTGGTGTACGTTGACGATCACATCC |
| | | | | ATGCGTGGGGGCATATGACCATCAGCACAGC |
| | | | | TGCCCAGTACCGCAATGCCGTCGTGGAGCAGC |
| | | | | ACCTCCCCCAACGGCAGCCAGAACCAGTGGA |
| | | | | GCCCACTCGGCCTCATGTGCGAGCCTGA |
| 101 | HSV-2 | HSV-2 | Version | ATGGGGAGACTCACATCAGGCGTAGGAACCG |
| | gD - | gE | 2.1 | CTGCCCTGTTGGTCGTGGCCGTTGGTCTGAGA |
| | KYA | | | GTTGTGTGTGCCAAATATGCTCGCACCTCTTG |
| | | | | GAAACGCGTTACTTCCGGGGAGGACGTTGTCC |
| | | | | TCCTTCCAGCACCCGCAGGACCTGAAGAGAG |
| | | | | GACTAGGGCCCACAAGCTGCTGTGGGCCGCT |
| | | | | GAACCTCTGGATGCCTGTGGTCCTCTGAGACC |
| | | | | TAGCTGGGTCGCCCTTTGGCCACCTAGACGCG |
| | | | | TTCTGGAGACGGTCGTGGATGCCGCGTGCATG |
| | | | | CGTGCACCCGAACCTCTGGCCATCGCCTATAG |
| | | | | TCCCCCTTTTCCCGCTGGCGACGAGGGGCTTT |
| | | | | ACTCCGAACTGGCCTGGCGGGATAGGGTGGC |
| | | | | GGTGGTGAACGAGAGCCTCGTCATCTACGGTG |
| | | | | CTCTGGAAACCGACTCAGGACTGTATACGCTC |
| | | | | AGCGTTGTTGGCCTCTCCGATGAGGCTCGACA |
| | | | | GGTTGCCTCCGTAGTGCTGGTCGTAGAACCAG |
| | | | | CCCCCGTACCAACACCCACACCCGACGACTAC |
| | | | | GACGAAGAGGACGACGCTGGAGTTAGCGAAA |
| | | | | GAACACCGGTGAGTGTGCCACCTCCCACACCG |
| | | | | CCAAGGAGACCCCCAGTAGCACCTCCAACCC |
| | | | | ATCCGAGAGTGATTCCCGAGGTCAGCCATGTG |
| | | | | CGCGGCGTAACTGTGCACATGGAGACGCCCG |
| | | | | AAGCGATACTGTTTGCCCCTGGAGAGACATTC |
| | | | | GGCACCAATGTGTCCATACACGCAATTGCGCA |
| | | | | CGATGATGGCCCATACGCTATGGACGTCGTCT |
| | | | | GGATGAGGTTCGATGTGCCTTCTTCTTGCGCC |
| | | | | GAGATGAGGATCTACGAGGCATGCCTGTATC |
| | | | | ACCCCCAATTGCCGGAGTGTCTGTCTCCCGCA |
| | | | | GATGCACCGTGTGCAGTGAGTAGCTGGGCTTA |
| | | | | TCGGTTGGCTGTCCGGAGTTATGCTGGGTGTT |
| | | | | CACGGACCACCCCACCTCCACGTTGCTTTGCT |
| | | | | GAAGCCAGAATGGAACCCGTGCCTGGTCTGG |
| | | | | CTTGGCTGGCATCAACTGTCAACCTGGAGTTC |
| | | | | CAGCATGCCTCTCCACAGCACGCAGGCCTGTA |
| | | | | TCTCTGCGTGGTGTACGTTGACGATCACATCC |
| | | | | ATGCGTGGGGGCATATGACCATCAGCACAGC |
| | | | | TGCCCAGTACCGCAATGCCGTCGTGGAGCAGC |
| | | | | ACCTCCCCCAACGGCAGCCAGAACCAGTGGA |
| | | | | GCCCACTCGGCCTCATGTGCGAGCCTGA |
| 347 | HSV-2 | HSV-2 | Version | ATGGGGAGACTCACATCAGGCGTAGGAACCG |
| | gD - | gE | 2.2 | CTGCCCTGTTGGTCGTGGCCGTTGGTCTGAGA |
| | KYA | | | GTTGTGTGTGCCAAATATGCTCGCACCTCTTG |
| | | | | GAAACGCGTTACTTCCGGGGAGGACGTTGTCC |
| | | | | TCCTTCCAGCACCCGCAGGACCTGAGGAAAG |
| | | | | GACTAGGGCCCACAAGCTGCTGTGGGCCGCT |
| | | | | GAACCTCTGGATGCCTGTGGTCCTCTGAGACC |
| | | | | TAGCTGGGTCGCCCTTTGGCCACCTAGACGCG |
| | | | | TTCTGGAGACGGTCGTGGATGCCGCGTGCATG |
| | | | | CGTGCACCCGAACCTCTGGCCATCGCCTATAG |
| | | | | TCCCCCTTTTCCCGCTGGCGACGAGGGGCTTT |
| | | | | ACTCCGAACTGGCCTGGCGGGATAGGGTGGC |
| | | | | GGTGGTGAACGAGAGCCTCGTCATCTACGGTG |
| | | | | CTCTGGAAACCGACTCAGGACTGTATACGCTC |
| | | | | AGCGTTGTTGGCCTCTCCGATGAGGCTCGACA |
| | | | | GGTTGCCTCCGTAGTGCTGGTCGTAGAACCAG |
| | | | | CCCCCGTACCAACACCCACACCCGACGACTAC |
| | | | | GACGAGGAGGACGACGCTGGAGTTAGCGAAA |
| | | | | GAACACCGGTGAGTGTGCCACCTCCCACACCG |
| | | | | CCAAGGAGACCCCCAGTAGCACCTCCAACCC |
| | | | | ATCCGAGAGTGATTCCCGAGGTCAGCCATGTG |
| | | | | CGCGGCGTAACTGTGCACATGGAGACGCCCG |
| | | | | AAGCGATACTGTTTGCCCCTGGAGAGACATTC |
| | | | | GGCACCAATGTGTCCATACACGCAATTGCGCA |
| | | | | CGATGATGGCCCATACGCTATGGACGTCGTCT |
| | | | | GGATGAGGTTCGATGTGCCTTCTTCTTGCGCC |
| | | | | GAGATGAGGATCTACGAGGCATGCCTGTATC |
| | | | | ACCCCCAATTGCCGGAGTGTCTGTCTCCCGCA |
| | | | | GATGCACCGTGTGCAGTGAGTAGCTGGGCTTA |
| | | | | TCGGTTGGCTGTCCGGAGTTATGCTGGGTGTT |
| | | | | CACGGACCACCCCACCTCCACGTTGCTTTGCT |
| | | | | GAAGCCAGAATGGAACCCGTGCCTGGTCTGG |
| | | | | CTTGGCTGGCATCAACTGTCAACCTGGAGTTC |
| | | | | CAGCATGCCTCTCCACAGCACGCAGGCCTGTA |
| | | | | TCTCTGCGTGGTGTACGTTGACGATCACATCC |
| | | | | ATGCGTGGGGGCATATGACCATCAGCACAGC |
| | | | | TGCCCAGTACCGCAATGCCGTCGTGGAGCAGC |
| | | | | ACCTCCCCCAACGGCAGCCAGAACCAGTGGA |
| | | | | GCCCACTCGGCCTCATGTGCGAGCCTGATAA |
| 102 | HSV-2 | HSV-2 | Version | ATGGGCAGACTGACCTCCGGCGTGGGCACCG |
| | gD - | gE | 3 | CCGCCCTGCTGGTGGTGGCCGTGGGCCTGAGA |
| | KYA | | | GTGGTGTGCGCCAAATACGCCAGAACCTCCTG |
| | | | | GAAAAGAGTGACCTCCGGCGAAGATGTGGTG |
| | | | | CTGCTGCCCGCCCCCGCCGGCCCCGAAGAAA |
| | | | | GAACCAGAGCCCACAAACTGCTGTGGGCCGC |
| | | | | CGAACCCCTGGATGCCTGCGGCCCCCTCAGAC |
| | | | | CCTCCTGGGTGGCCCTGTGGCCCCCTAGAAGG |
| | | | | GTGCTGGAAACCGTGGTGGATGCCGCCTGCAT |
| | | | | GAGAGCCCCCGAACCCCTGGCCATCGCCTACT |
| | | | | CCCCTCCCTTCCCCGCCGGCGATGAAGGCCTG |
| | | | | TACTCCGAACTGGCCTGGAGAGATAGAGTGG |
| | | | | CCGTGGTGAACGAATCCCTGGTGATCTACGGC |
| | | | | GCCCTGGAAACCGATTCCGGCCTGTACACCCT |
| | | | | GTCCGTGGTGGGCCTGTCCGATGAAGCCAGAC |
| | | | | AGGTGGCCTCCGTGGTGCTGGTGGTGGAACCC |
| | | | | GCCCCCGTGCCCACCCCCACCCCCGATGATTA |
| | | | | CGATGAAGAAGATGATGCCGGCGTGTCCGAA |
| | | | | AGAACCCCCGTGTCCGTGCCCCCTCCCACCCC |
| | | | | TCCCCGCAGACCCCCTGTGGCCCCTCCCACCC |
| | | | | ACCCCAGAGTGATCCCCGAAGTGTCCCACGTG |
| | | | | AGAGGCGTGACCGTGCACATGGAAACCCCCG |
| | | | | AAGCCATCCTGTTCGCCCCCGGCGAAACCTTC |
| | | | | GGCACCAACGTGTCCATCCACGCCATCGCCCA |
| | | | | CGATGATGGCCCCTACGCCATGGATGTGGTGT |
| | | | | GGATGAGATTCGATGTGCCCTCCTCCTGCGCC |
| | | | | GAAATGAGAATCTACGAAGCCTGCCTGTACC |
| | | | | ACCCCCAGCTGCCCGAATGCCTGTCCCCCGCC |
| | | | | GATGCCCCCTGCGCCGTGTCCTCCTGGGCCTA |
| | | | | CAGACTGGCCGTGAGATCCTACGCCGGCTGCT |
| | | | | CCAGAACCACCCCTCCCCCTAGATGCTTCGCC |
| | | | | GAAGCCAGAATGGAACCCGTGCCCGGCCTGG |
| | | | | CCTGGCTGGCCTCCACCGTGAACCTGGAATTC |
| | | | | CAGCACGCCAGCCCCCAGCACGCCGGCCTGT |
| | | | | ACCTGTGCGTGGTGTACGTGGATGATCACATC |
| | | | | CACGCCTGGGGCCACATGACCATCTCCACCGC |
| | | | | CGCCCAGTACAGAAACGCCGTGGTGGAACAG |
| | | | | CACCTGCCCCAGAGACAGCCCGAACCCGTGG |
| | | | | AACCCACCAGACCCCACGTGAGAGCCTGA |
| 103 | HSV-2 | HSV-2 | Version | ATGGCACGGGGAGCCGGATTGGTGTTCTTTGT |
| | gE | gE | 2 | GGGCGTGTGGGTGGTGAGCTGCTTGGCAGCC |
| | | | | GCACCACGCACCTCTTGGAAACGCGTTACTTC |
| | | | | CGGGGAGGACGTTGTCCTCCTTCCAGCACCCG |
| | | | | CAGGACCTGAGGAAAGGACTAGGGCCCACAA |
| | | | | GCTGCTGTGGGCCGCTGAACCTCTGGATGCCT |
| | | | | GTGGTCCTCTGAGACCTAGCTGGGTCGCCCTT |
| | | | | TGGCCACCTAGACGCGTTCTGGAGACGGTCGT |
| | | | | GGATGCCGCGTGCATGCGTGCACCCGAACCTC |
| | | | | TGGCCATCGCCTATAGTCCCCCTTTTCCCGCTG |
| | | | | GCGACGAGGGGCTTTACTCCGAACTGGCCTGG |
| | | | | CGGGATAGGGTGGCGGTGGTGAACGAGAGCC |
| | | | | TCGTCATCTACGGTGCTCTGGAAACCGACTCA |
| | | | | GGACTGTATACGCTCAGCGTTGTTGGCCTCTC |
| | | | | CGATGAGGCTCGACAGGTTGCCTCCGTAGTGC |
| | | | | TGGTCGTAGAACCAGCCCCCGTACCAACACCC |
| | | | | ACACCCGACGACTACGACGAGGAGGACGACG |
| | | | | CTGGAGTTAGCGAAAGAACACCGGTGAGTGT |
| | | | | GCCACCTCCCACACCGCCAAGGAGACCCCCA |
| | | | | GTAGCACCTCCAACCCATCCGAGAGTGATTCC |
| | | | | CGAGGTCAGCCATGTGCGCGGCGTAACTGTGC |
| | | | | ACATGGAGACGCCCGAAGCGATACTGTTTGCC |
| | | | | CCTGGAGAGACATTCGGCACCAATGTGTCCAT |
| | | | | ACACGCAATTGCGCACGATGATGGCCCATAC |
| | | | | GCTATGGACGTCGTCTGGATGAGGTTCGATGT |
| | | | | GCCTTCTTCTTGCGCCGAGATGAGGATCTACG |
| | | | | AGGCATGCCTGTATCACCCCCAATTGCCGGAG |
| | | | | TGTCTGTCTCCCGCAGATGCACCGTGTGCAGT |
| | | | | GAGTAGCTGGGCTTATCGGTTGGCTGTCCGGA |
| | | | | GTTATGCTGGGTGTTCACGGACCACCCCACCT |
| | | | | CCACGTTGCTTTGCTGAAGCCAGAATGGAACC |
| | | | | CGTGCCTGGTCTGGCTTGGCTGGCATCAACTG |
| | | | | TCAACCTGGAGTTCCAGCATGCCTCTCCACAG |
| | | | | CACGCAGGCCTGTATCTCTGCGTGGTGTACGT |
| | | | | TGACGATCACATCCATGCGTGGGGGCATATGA |
| | | | | CCATCAGCACAGCTGCCCAGTACCGCAATGCC |
| | | | | GTCGTGGAGCAGCACCTCCCCCAACGGCAGC |
| | | | | CAGAACCAGTGGAGCCCACTCGGCCTCATGTG |
| | | | | CGAGCCTGA |
| 313 | HSV-2 | HSV-2 | Version | ATGGCACGGGGCGCAGGTTTGGTCTTTTTCGT |
| | gE | gE | 2.1 | GGGCGTGTGGGTGGTGAGCTGCTTGGCAGCC |
| | | | | GCACCACGCACCTCTTGGAAACGCGTTACTTC |
| | | | | CGGGGAGGACGTTGTCCTCCTTCCAGCACCCG |
| | | | | CAGGACCTGAGGAAAGGACTAGGGCCCACAA |
| | | | | GCTGCTGTGGGCCGCTGAACCTCTGGATGCCT |
| | | | | GTGGTCCTCTGAGACCTAGCTGGGTCGCCCTT |
| | | | | TGGCCACCTAGACGCGTTCTGGAGACGGTCGT |
| | | | | GGATGCCGCGTGCATGCGTGCACCCGAACCTC |
| | | | | TGGCCATCGCCTATAGTCCCCCTTTTCCCGCTG |
| | | | | GCGACGAGGGGCTTTACTCCGAACTGGCCTGG |
| | | | | CGGGATAGGGTGGCGGTGGTGAACGAGAGCC |
| | | | | TCGTCATCTACGGTGCTCTGGAAACCGACTCA |
| | | | | GGACTGTATACGCTCAGCGTTGTTGGCCTCTC |
| | | | | CGATGAGGCTCGACAGGTTGCCTCCGTAGTGC |
| | | | | TGGTCGTAGAACCAGCCCCCGTACCAACACCC |
| | | | | ACACCCGACGACTACGACGAGGAGGACGACG |
| | | | | CTGGAGTTAGCGAAAGAACACCGGTGAGTGT |
| | | | | GCCACCTCCCACACCGCCAAGGAGACCCCCA |
| | | | | GTAGCACCTCCAACCCATCCGAGAGTGATTCC |
| | | | | CGAGGTCAGCCATGTGCGCGGCGTAACTGTGC |
| | | | | ACATGGAGACGCCCGAAGCGATACTGTTTGCC |
| | | | | CCTGGAGAGACATTCGGCACCAATGTGTCCAT |
| | | | | ACACGCAATTGCGCACGATGATGGCCCATAC |
| | | | | GCTATGGACGTCGTCTGGATGAGGTTCGATGT |
| | | | | GCCTTCTTCTTGCGCCGAGATGAGGATCTACG |
| | | | | AGGCATGCCTGTATCACCCCCAATTGCCGGAG |
| | | | | TGTCTGTCTCCCGCAGATGCACCGTGTGCAGT |
| | | | | GAGTAGCTGGGCTTATCGGTTGGCTGTCCGGA |
| | | | | GTTATGCTGGGTGTTCACGGACCACCCCACCT |
| | | | | CCACGTTGCTTTGCTGAAGCCAGAATGGAACC |
| | | | | CGTGCCTGGTCTGGCTTGGCTGGCATCAACTG |
| | | | | TCAACCTGGAGTTCCAGCATGCCTCTCCACAG |
| | | | | CACGCAGGCCTGTATCTCTGCGTGGTGTACGT |
| | | | | TGACGATCACATCCATGCGTGGGGGCATATGA |
| | | | | CCATCAGCACAGCTGCCCAGTACCGCAATGCC |
| | | | | GTCGTGGAGCAGCACCTCCCCCAACGGCAGC |
| | | | | CAGAACCAGTGGAGCCCACTCGGCCTCATGTG |
| | | | | CGAGCCTGA |
| 314 | HSV-2 | HSV-2 | Version | ATGGCGAGAGGAGCCGGACTCGTGTTCTTTGT |
| | gE | gE | 2.2 | GGGAGTCTGGGTTGTGAGCTGCCTGGCAGCA |
| | | | | GCTCCACGCACCTCTTGGAAACGCGTTACTTC |
| | | | | CGGGGAGGACGTTGTCCTCCTTCCAGCACCCG |
| | | | | CAGGACCTGAAGAGAGGACTAGGGCCCACAA |
| | | | | GCTGCTGTGGGCCGCTGAACCTCTGGATGCCT |
| | | | | GTGGTCCTCTGAGACCTAGCTGGGTCGCCCTT |
| | | | | TGGCCACCTAGACGCGTTCTGGAGACGGTCGT |
| | | | | GGATGCCGCGTGCATGCGTGCACCCGAACCTC |
| | | | | TGGCCATCGCCTATAGTCCCCCTTTTCCCGCTG |
| | | | | GCGACGAGGGGCTTTACTCCGAACTGGCCTGG |
| | | | | CGGGATAGGGTGGCGGTGGTGAACGAGAGCC |
| | | | | TCGTCATCTACGGTGCTCTGGAAACCGACTCA |
| | | | | GGACTGTATACGCTCAGCGTTGTTGGCCTCTC |
| | | | | CGATGAGGCTCGACAGGTTGCCTCCGTAGTGC |
| | | | | TGGTCGTAGAACCAGCCCCCGTACCAACACCC |
| | | | | ACACCCGACGACTACGACGAAGAGGACGACG |
| | | | | CTGGAGTTAGCGAAAGAACACCGGTGAGTGT |
| | | | | GCCACCTCCCACACCGCCAAGGAGACCCCCA |
| | | | | GTAGCACCTCCAACCCATCCGAGAGTGATTCC |
| | | | | CGAGGTCAGCCATGTGCGCGGCGTAACTGTGC |
| | | | | ACATGGAGACGCCCGAAGCGATACTGTTTGCC |
| | | | | CCTGGAGAGACATTCGGCACCAATGTGTCCAT |
| | | | | ACACGCAATTGCGCACGATGATGGCCCATAC |
| | | | | GCTATGGACGTCGTCTGGATGAGGTTCGATGT |
| | | | | GCCTTCTTCTTGCGCCGAGATGAGGATCTACG |
| | | | | AGGCATGCCTGTATCACCCCCAATTGCCGGAG |
| | | | | TGTCTGTCTCCCGCAGATGCACCGTGTGCAGT |
| | | | | GAGTAGCTGGGCTTATCGGTTGGCTGTCCGGA |
| | | | | GTTATGCTGGGTGTTCACGGACCACCCCACCT |
| | | | | CCACGTTGCTTTGCTGAAGCCAGAATGGAACC |
| | | | | CGTGCCTGGTCTGGCTTGGCTGGCATCAACTG |
| | | | | TCAACCTGGAGTTCCAGCATGCCTCTCCACAG |
| | | | | CACGCAGGCCTGTATCTCTGCGTGGTGTACGT |
| | | | | TGACGATCACATCCATGCGTGGGGGCATATGA |
| | | | | CCATCAGCACAGCTGCCCAGTACCGCAATGCC |
| | | | | GTCGTGGAGCAGCACCTCCCCCAACGGCAGC |
| | | | | CAGAACCAGTGGAGCCCACTCGGCCTCATGTG |
| | | | | CGAGCCTGA |
| 315 | HSV-2 | HSV-2 | Version | ATGGCGAGAGGAGCCGGGCTCGTGTTCTTTGT |
| | gE | gE | 2.3 | GGGCGTATGGGTCGTTTCCTGCCTGGCTGCCG |
| | | | | CACCCCGCACCTCTTGGAAACGCGTTACTTCC |
| | | | | GGGGAGGACGTTGTCCTCCTTCCAGCACCCGC |
| | | | | AGGACCTGAGGAAAGGACTAGGGCCCACAAG |
| | | | | CTGCTGTGGGCCGCTGAACCTCTGGATGCCTG |
| | | | | TGGTCCTCTGAGACCTAGCTGGGTCGCCCTTT |
| | | | | GGCCACCTAGACGCGTTCTGGAGACGGTCGTG |
| | | | | GATGCCGCGTGCATGCGTGCACCCGAACCTCT |
| | | | | GGCCATCGCCTATAGTCCCCCTTTTCCCGCTG |
| | | | | GCGACGAGGGGCTTTACTCCGAACTGGCCTGG |
| | | | | CGGGATAGGGTGGCGGTGGTGAACGAGAGCC |
| | | | | TCGTCATCTACGGTGCTCTGGAAACCGACTCA |
| | | | | GGACTGTATACGCTCAGCGTTGTTGGCCTCTC |
| | | | | CGATGAGGCTCGACAGGTTGCCTCCGTAGTGC |
| | | | | TGGTCGTAGAACCAGCCCCCGTACCAACACCC |
| | | | | ACACCCGACGACTACGACGAGGAGGACGACG |
| | | | | CTGGAGTTAGCGAAAGAACACCGGTGAGTGT |
| | | | | GCCACCTCCCACACCGCCAAGGAGACCCCCA |
| | | | | GTAGCACCTCCAACCCATCCGAGAGTGATTCC |
| | | | | CGAGGTCAGCCATGTGCGCGGCGTAACTGTGC |
| | | | | ACATGGAGACGCCCGAAGCGATACTGTTTGCC |
| | | | | CCTGGAGAGACATTCGGCACCAATGTGTCCAT |
| | | | | ACACGCAATTGCGCACGATGATGGCCCATAC |
| | | | | GCTATGGACGTCGTCTGGATGAGGTTCGATGT |
| | | | | GCCTTCTTCTTGCGCCGAGATGAGGATCTACG |
| | | | | AGGCATGCCTGTATCACCCCCAATTGCCGGAG |
| | | | | TGTCTGTCTCCCGCAGATGCACCGTGTGCAGT |
| | | | | GAGTAGCTGGGCTTATCGGTTGGCTGTCCGGA |
| | | | | GTTATGCTGGGTGTTCACGGACCACCCCACCT |
| | | | | CCACGTTGCTTTGCTGAAGCCAGAATGGAACC |
| | | | | CGTGCCTGGTCTGGCTTGGCTGGCATCAACTG |
| | | | | TCAACCTGGAGTTCCAGCATGCCTCTCCACAG |
| | | | | CACGCAGGCCTGTATCTCTGCGTGGTGTACGT |
| | | | | TGACGATCACATCCATGCGTGGGGGCATATGA |
| | | | | CCATCAGCACAGCTGCCCAGTACCGCAATGCC |
| | | | | GTCGTGGAGCAGCACCTCCCCCAACGGCAGC |
| | | | | CAGAACCAGTGGAGCCCACTCGGCCTCATGTG |
| | | | | CGAGCCTGA |
| 316 | HSV-2 | HSV-2 | Version | ATGGCGAGAGGAGCCGGACTCGTGTTCTTTGT |
| | gE - | gE | 2 | GGGAGTCTGGGTTGTGAGCTGCCTGGTAGTAG |
| | LVVV | | | TTCCACGCACCTCTTGGAAACGCGTTACTTCC |
| | | | | GGGGAGGACGTTGTCCTCCTTCCAGCACCCGC |
| | | | | AGGACCTGAAGAGAGGACTAGGGCCCACAAG |
| | | | | CTGCTGTGGGCCGCTGAACCTCTGGATGCCTG |
| | | | | TGGTCCTCTGAGACCTAGCTGGGTCGCCCTTT |
| | | | | GGCCACCTAGACGCGTTCTGGAGACGGTCGTG |
| | | | | GATGCCGCGTGCATGCGTGCACCCGAACCTCT |
| | | | | GGCCATCGCCTATAGTCCCCCTTTTCCCGCTG |
| | | | | GCGACGAGGGGCTTTACTCCGAACTGGCCTGG |
| | | | | CGGGATAGGGTGGCGGTGGTGAACGAGAGCC |
| | | | | TCGTCATCTACGGTGCTCTGGAAACCGACTCA |
| | | | | GGACTGTATACGCTCAGCGTTGTTGGCCTCTC |
| | | | | CGATGAGGCTCGACAGGTTGCCTCCGTAGTGC |
| | | | | TGGTCGTAGAACCAGCCCCCGTACCAACACCC |
| | | | | ACACCCGACGACTACGACGAAGAGGACGACG |
| | | | | CTGGAGTTAGCGAAAGAACACCGGTGAGTGT |
| | | | | GCCACCTCCCACACCGCCAAGGAGACCCCCA |
| | | | | GTAGCACCTCCAACCCATCCGAGAGTGATTCC |
| | | | | CGAGGTCAGCCATGTGCGCGGCGTAACTGTGC |
| | | | | ACATGGAGACGCCCGAAGCGATACTGTTTGCC |
| | | | | CCTGGAGAGACATTCGGCACCAATGTGTCCAT |
| | | | | ACACGCAATTGCGCACGATGATGGCCCATAC |
| | | | | GCTATGGACGTCGTCTGGATGAGGTTCGATGT |
| | | | | GCCTTCTTCTTGCGCCGAGATGAGGATCTACG |
| | | | | AGGCATGCCTGTATCACCCCCAATTGCCGGAG |
| | | | | TGTCTGTCTCCCGCAGATGCACCGTGTGCAGT |
| | | | | GAGTAGCTGGGCTTATCGGTTGGCTGTCCGGA |
| | | | | GTTATGCTGGGTGTTCACGGACCACCCCACCT |
| | | | | CCACGTTGCTTTGCTGAAGCCAGAATGGAACC |
| | | | | CGTGCCTGGTCTGGCTTGGCTGGCATCAACTG |
| | | | | TCAACCTGGAGTTCCAGCATGCCTCTCCACAG |
| | | | | CACGCAGGCCTGTATCTCTGCGTGGTGTACGT |
| | | | | TGACGATCACATCCATGCGTGGGGGCATATGA |
| | | | | CCATCAGCACAGCTGCCCAGTACCGCAATGCC |
| | | | | GTCGTGGAGCAGCACCTCCCCCAACGGCAGC |
| | | | | CAGAACCAGTGGAGCCCACTCGGCCTCATGTG |
| | | | | CGAGCCTGA |
| 317 | None | HSV-2 | Version | ATGCGCACCTCTTGGAAACGCGTTACTTCCGG |
| | | gE | 2 | GGAGGACGTTGTCCTCCTTCCAGCACCCGCAG |
| | | | | GACCTGAGGAAAGGACTAGGGCCCACAAGCT |
| | | | | GCTGTGGGCCGCTGAACCTCTGGATGCCTGTG |
| | | | | GTCCTCTGAGACCTAGCTGGGTCGCCCTTTGG |
| | | | | CCACCTAGACGCGTTCTGGAGACGGTCGTGGA |
| | | | | TGCCGCGTGCATGCGTGCACCCGAACCTCTGG |
| | | | | CCATCGCCTATAGTCCCCCTTTTCCCGCTGGC |
| | | | | GACGAGGGGCTTTACTCCGAACTGGCCTGGCG |
| | | | | GGATAGGGTGGCGGTGGTGAACGAGAGCCTC |
| | | | | GTCATCTACGGTGCTCTGGAAACCGACTCAGG |
| | | | | ACTGTATACGCTCAGCGTTGTTGGCCTCTCCG |
| | | | | ATGAGGCTCGACAGGTTGCCTCCGTAGTGCTG |
| | | | | GTCGTAGAACCAGCCCCCGTACCAACACCCAC |
| | | | | ACCCGACGACTACGACGAGGAGGACGACGCT |
| | | | | GGAGTTAGCGAAAGAACACCGGTGAGTGTGC |
| | | | | CACCTCCCACACCGCCAAGGAGACCCCCAGT |
| | | | | AGCACCTCCAACCCATCCGAGAGTGATTCCCG |
| | | | | AGGTCAGCCATGTGCGCGGCGTAACTGTGCAC |
| | | | | ATGGAGACGCCCGAAGCGATACTGTTTGCCCC |
| | | | | TGGAGAGACATTCGGCACCAATGTGTCCATAC |
| | | | | ACGCAATTGCGCACGATGATGGCCCATACGCT |
| | | | | ATGGACGTCGTCTGGATGAGGTTCGATGTGCC |
| | | | | TTCTTCTTGCGCCGAGATGAGGATCTACGAGG |
| | | | | CATGCCTGTATCACCCCCAATTGCCGGAGTGT |
| | | | | CTGTCTCCCGCAGATGCACCGTGTGCAGTGAG |
| | | | | TAGCTGGGCTTATCGGTTGGCTGTCCGGAGTT |
| | | | | ATGCTGGGTGTTCACGGACCACCCCACCTCCA |
| | | | | CGTTGCTTTGCTGAAGCCAGAATGGAACCCGT |
| | | | | GCCTGGTCTGGCTTGGCTGGCATCAACTGTCA |
| | | | | ACCTGGAGTTCCAGCATGCCTCTCCACAGCAC |
| | | | | GCAGGCCTGTATCTCTGCGTGGTGTACGTTGA |
| | | | | CGATCACATCCATGCGTGGGGGCATATGACCA |
| | | | | TCAGCACAGCTGCCCAGTACCGCAATGCCGTC |
| | | | | GTGGAGCAGCACCTCCCCCAACGGCAGCCAG |
| | | | | AACCAGTGGAGCCCACTCGGCCTCATGTGCGA |
| | | | | GCCTGA |
| 185 | HSV-1 | HSV-2 | Version | ATGGGCGGAGCTGCTGCTAGACTGGGAGCCG |
| | gD | gE | 1 | TGATCCTGTTCGTGGTTATCGTGGGACTGCAT |
| | | | | GGCGTGCGGGGCAAGTATAGAACCTCTTGGA |
| | | | | AGCGCGTGACAAGCGGCGAGGATGTGGTTCT |
| | | | | GCTTCCTGCTCCTGCCGGACCTGAGGAAAGAA |
| | | | | CAAGAGCCCACAAGCTGCTGTGGGCCGCTGA |
| | | | | ACCTTTGGATGCCTGTGGACCTCTGAGGCCTT |
| | | | | CTTGGGTTGCACTGTGGCCACCTCGGAGAGTG |
| | | | | CTGGAAACAGTGGTGGATGCCGCCTGCATGA |
| | | | | GAGCCCCTGAACCTCTGGCCATTGCCTACTCT |
| | | | | CCACCTTTTCCAGCCGGCGACGAGGGCCTGTA |
| | | | | TTCTGAGCTTGCTTGGAGAGACAGAGTGGCCG |
| | | | | TGGTCAACGAGAGCCTGGTTATCTATGGCGCC |
| | | | | CTGGAAACCGACAGCGGCCTGTACACACTGTC |
| | | | | TGTCGTGGGCCTGTCTGACGAGGCTAGACAGG |
| | | | | TGGCATCTGTGGTGCTGGTGGTGGAACCTGCT |
| | | | | CCAGTGCCTACACCTACACCTGACGACTACGA |
| | | | | CGAGGAAGATGACGCTGGCGTCAGCGAGAGA |
| | | | | ACCCCTGTTTCTGTGCCTCCTCCTACGCCTCCT |
| | | | | CGTAGACCTCCTGTTGCTCCTCCAACACACCC |
| | | | | CAGAGTGATCCCTGAAGTGTCTCACGTGCGGG |
| | | | | GCGTGACCGTGCACATGGAAACACCTGAGGC |
| | | | | CATCCTGTTCGCCCCTGGCGAGACATTTGGCA |
| | | | | CCAACGTGTCCATCCACGCTATCGCCCACGAC |
| | | | | GATGGCCCTTACGCCATGGATGTCGTGTGGAT |
| | | | | GAGATTCGACGTGCCCAGCAGCTGCGCCGAG |
| | | | | ATGAGAATCTACGAGGCCTGCCTGTATCACCC |
| | | | | TCAGCTGCCCGAATGTCTGAGCCCTGCTGATG |
| | | | | CCCCTTGTGCCGTTAGCAGCTGGGCCTATAGA |
| | | | | CTGGCCGTGCGGTCTTATGCCGGCTGCTCTAG |
| | | | | AACAACCCCTCCTCCTCGGTGTTTCGCCGAGG |
| | | | | CCAGAATGGAACCTGTTCCTGGACTGGCCTGG |
| | | | | CTGGCCTCCACAGTGAACCTGGAATTTCAGCA |
| | | | | CGCCTCTCCACAGCACGCCGGCCTGTATCTGT |
| | | | | GTGTGGTGTACGTGGACGATCACATCCACGCC |
| | | | | TGGGGCCACATGACCATCTCTACAGCCGCTCA |
| | | | | GTACCGGAACGCCGTGGTTGAACAGCATCTGC |
| | | | | CCCAGAGACAGCCCGAGCCTGTGGAACCTAC |
| | | | | AAGACCTCATGTTCGGGCCTGATAA |
| 186 | HSV-1 | HSV-2 | Version | ATGGGAGGAGCAGCTGCCAGACTCGGTGCCG |
| | gD | gE | 2 | TGATCCTGTTCGTGGTCATTGTTGGCCTGCAC |
| | | | | GGGGTAAGGGGCAAGTACCGCACCTCTTGGA |
| | | | | AACGCGTTACTTCCGGGGAGGACGTTGTCCTC |
| | | | | CTTCCAGCACCCGCAGGACCTGAGGAAAGGA |
| | | | | CTAGGGCCCACAAGCTGCTGTGGGCCGCTGA |
| | | | | ACCTCTGGATGCCTGTGGTCCTCTGAGACCTA |
| | | | | GCTGGGTCGCCCTTTGGCCACCTAGACGCGTT |
| | | | | CTGGAGACGGTCGTGGATGCCGCGTGCATGC |
| | | | | GTGCACCCGAACCTCTGGCCATCGCCTATAGT |
| | | | | CCCCCTTTTCCCGCTGGCGACGAGGGGCTTTA |
| | | | | CTCCGAACTGGCCTGGCGGGATAGGGTGGCG |
| | | | | GTGGTGAACGAGAGCCTCGTCATCTACGGTGC |
| | | | | TCTGGAAACCGACTCAGGACTGTATACGCTCA |
| | | | | GCGTTGTTGGCCTCTCCGATGAGGCTCGACAG |
| | | | | GTTGCCTCCGTAGTGCTGGTCGTAGAACCAGC |
| | | | | CCCCGTACCAACACCCACACCCGACGACTACG |
| | | | | ACGAGGAGGACGACGCTGGAGTTAGCGAAAG |
| | | | | AACACCGGTGAGTGTGCCACCTCCCACACCGC |
| | | | | CAAGGAGACCCCCAGTAGCACCTCCAACCCA |
| | | | | TCCGAGAGTGATTCCCGAGGTCAGCCATGTGC |
| | | | | GCGGCGTAACTGTGCACATGGAGACGCCCGA |
| | | | | AGCGATACTGTTTGCCCCTGGAGAGACATTCG |
| | | | | GCACCAATGTGTCCATACACGCAATTGCGCAC |
| | | | | GATGATGGCCCATACGCTATGGACGTCGTCTG |
| | | | | GATGAGGTTCGATGTGCCTTCTTCTTGCGCCG |
| | | | | AGATGAGGATCTACGAGGCATGCCTGTATCAC |
| | | | | CCCCAATTGCCGGAGTGTCTGTCTCCCGCAGA |
| | | | | TGCACCGTGTGCAGTGAGTAGCTGGGCTTATC |
| | | | | GGTTGGCTGTCCGGAGTTATGCTGGGTGTTCA |
| | | | | CGGACCACCCCACCTCCACGTTGCTTTGCTGA |
| | | | | AGCCAGAATGGAACCCGTGCCTGGTCTGGCTT |
| | | | | GGCTGGCATCAACTGTCAACCTGGAGTTCCAG |
| | | | | CATGCCTCTCCACAGCACGCAGGCCTGTATCT |
| | | | | CTGCGTGGTGTACGTTGACGATCACATCCATG |
| | | | | CGTGGGGGCATATGACCATCAGCACAGCTGC |
| | | | | CCAGTACCGCAATGCCGTCGTGGAGCAGCAC |
| | | | | CTCCCCCAACGGCAGCCAGAACCAGTGGAGC |
| | | | | CCACTCGGCCTCATGTGCGAGCCTGATAA |
| 187 | HSV-1 | HSV-2 | Version | ATGGGAGGAGCTGCTGCTAGATTGGGAGCTG |
| | gD | gE | 3 | TGATTCTGTTTGTGGTGATTGTGGGACTGCAT |
| | | | | GGAGTGAGAGGAAAATACAGAACCTCTTGGA |
| | | | | AAAGAGTGACCTCTGGAGAAGATGTGGTGCT |
| | | | | GCTGCCAGCTCCAGCTGGACCAGAAGAAAGA |
| | | | | ACCAGAGCACACAAACTGCTGTGGGCTGCTG |
| | | | | AACCTCTGGATGCTTGTGGACCTCTGAGACCT |
| | | | | TCTTGGGTGGCTCTGTGGCCACCAAGAAGGGT |
| | | | | GCTGGAAACAGTGGTGGATGCTGCTTGCATGA |
| | | | | GAGCACCTGAACCTCTGGCAATTGCATACTCT |
| | | | | CCTCCTTTTCCAGCTGGAGATGAAGGACTGTA |
| | | | | TTCTGAACTGGCTTGGAGAGACAGAGTGGCTG |
| | | | | TGGTGAATGAATCTCTGGTGATCTATGGAGCA |
| | | | | CTGGAAACAGATTCTGGACTGTACACCCTGTC |
| | | | | TGTGGTGGGACTGTCTGATGAAGCAAGACAG |
| | | | | GTGGCATCTGTGGTGCTGGTGGTGGAACCAGC |
| | | | | TCCAGTGCCAACCCCAACCCCAGATGATTATG |
| | | | | ATGAAGAAGATGATGCTGGAGTGTCTGAAAG |
| | | | | AACCCCAGTGTCTGTGCCACCACCAACCCCAC |
| | | | | CAAGAAGACCACCAGTGGCTCCACCAACCCA |
| | | | | CCCAAGAGTGATCCCAGAAGTGTCTCATGTGA |
| | | | | GAGGAGTGACAGTGCACATGGAAACCCCTGA |
| | | | | AGCAATCCTGTTTGCACCTGGAGAAACCTTTG |
| | | | | GAACCAATGTGTCCATCCATGCAATTGCACAT |
| | | | | GATGATGGACCTTATGCAATGGATGTGGTGTG |
| | | | | GATGAGATTTGATGTGCCTTCTTCTTGTGCTG |
| | | | | AAATGAGAATCTATGAAGCTTGCCTGTACCAC |
| | | | | CCTCAGCTGCCTGAATGCCTGTCTCCTGCTGA |
| | | | | TGCTCCTTGTGCTGTGTCTTCTTGGGCTTACAG |
| | | | | ACTGGCTGTGAGATCTTATGCTGGATGCTCCA |
| | | | | GAACCACCCCACCACCAAGATGCTTTGCTGAA |
| | | | | GCAAGAATGGAACCTGTGCCTGGACTGGCAT |
| | | | | GGCTGGCATCCACAGTGAATCTGGAATTTCAG |
| | | | | CATGCTTCTCCTCAGCATGCTGGACTGTACCT |
| | | | | GTGTGTGGTGTATGTGGACGATCACATCCATG |
| | | | | CTTGGGGACACATGACCATCAGCACAGCTGCT |
| | | | | CAGTACAGAAATGCTGTGGTGGAACAGCACC |
| | | | | TGCCACAGAGACAGCCAGAACCAGTGGAACC |
| | | | | AACCAGACCACATGTGAGAGCTTGATAA |
| 188 | HSV-1 | HSV-2 | Version | ATGGGCGGAGCTGCGGCTCGCCTCGGAGCCG |
| | gD | gE | 4 | TCATTCTGTTCGTGGTGATCGTGGGTCTGCAT |
| | | | | GGGGTCAGAGGAAAGTACCGGACCAGCTGGA |
| | | | | AAAGGGTGACATCTGGGGAAGATGTCGTGCT |
| | | | | CCTTCCTGCGCCTGCTGGCCCAGAAGAACGCA |
| | | | | CTAGGGCCCACAAGCTTCTGTGGGCCGCGGA |
| | | | | GCCACTGGATGCGTGTGGTCCCCTGCGACCCT |
| | | | | CTTGGGTGGCATTGTGGCCACCCCGACGCGTA |
| | | | | CTCGAAACGGTCGTTGACGCCGCCTGTATGAG |
| | | | | AGCGCCAGAGCCCCTCGCCATTGCCTACAGCC |
| | | | | CGCCTTTTCCCGCCGGTGACGAAGGACTGTAC |
| | | | | AGTGAGCTGGCCTGGCGCGATAGGGTCGCCG |
| | | | | TAGTTAACGAGTCCCTGGTGATATACGGCGCT |
| | | | | CTGGAAACCGACAGTGGCTTGTACACCCTGTC |
| | | | | CGTTGTAGGCCTGTCCGATGAAGCACGGCAA |
| | | | | GTGGCTTCCGTGGTACTGGTCGTAGAGCCCGC |
| | | | | ACCAGTTCCCACACCCACCCCGGATGACTACG |
| | | | | ATGAGGAGGACGATGCCGGTGTGAGTGAGCG |
| | | | | TACACCTGTTAGCGTACCTCCACCAACTCCTC |
| | | | | CACGCCGCCCTCCTGTTGCACCGCCTACACAT |
| | | | | CCCCGTGTGATTCCTGAAGTGTCACACGTTAG |
| | | | | AGGGGTGACAGTCCACATGGAGACTCCCGAA |
| | | | | GCCATCCTCTTTGCACCAGGCGAGACTTTTGG |
| | | | | GACCAATGTGAGCATTCACGCCATAGCTCACG |
| | | | | ATGACGGGCCCTATGCCATGGACGTGGTGTGG |
| | | | | ATGAGGTTCGATGTGCCCTCATCATGCGCTGA |
| | | | | GATGCGGATCTACGAAGCTTGCCTGTATCACC |
| | | | | CACAGCTTCCCGAGTGCTTGTCTCCCGCTGAC |
| | | | | GCCCCTTGTGCTGTTAGCTCTTGGGCTTATCG |
| | | | | GCTTGCCGTCAGGAGCTATGCTGGATGCTCCA |
| | | | | GAACCACACCTCCACCGAGGTGTTTCGCCGAG |
| | | | | GCCAGAATGGAGCCTGTGCCAGGACTGGCCT |
| | | | | GGCTGGCAAGTACTGTGAACCTGGAATTCCAG |
| | | | | CACGCATCACCTCAGCATGCAGGCCTGTACCT |
| | | | | CTGCGTTGTCTATGTCGACGACCATATCCACG |
| | | | | CATGGGGCCATATGACCATCAGCACGGCAGC |
| | | | | ACAGTATCGGAATGCTGTGGTCGAGCAGCACT |
| | | | | TGCCCCAGCGACAACCCGAACCAGTGGAGCC |
| | | | | AACCAGACCGCATGTGCGGGCCTGA |

TABLE 13
Example Ribonucleic Acid Sequences encoding secretory signals and HSV-2
glycoproteins

| SEQ ID | Secretory | Antigen/ | | |
|---|---|---|---|---|
| NO: | signal | OFR | Version | Sequences (Polyribonucleotide) |
| 104 | IL2 | HSV-2 | | AUGCGCAUGCAGCUGCUGCUGCUGAUCGCCC |
| | | gC | | UGUCCCUGGCCCUGGUGACCAACUCCGCCUCC |
| | | | | CCCGGCCGCACCAUCACCGUGGGCCCCCGCGG |
| | | | | CAACGCCUCCAACGCCGCCCCCUCCGCCUCCC |
| | | | | CCCGCAACGCCUCCGCCCCCCGCACCACCCCC |
| | | | | ACCCCCCCCCAGCCCCGCAAGGCCACCAAGUC |
| | | | | CAAGGCCUCCACCGCCAAGCCCGCCCCCCCCC |
| | | | | CCAAGACCGGCCCCCCCAAGACCUCCUCCGAG |
| | | | | CCCGUGCGCUGCAACCGCCACGACCCCCUGGC |
| | | | | CCGCUACGGCUCCCGCGUGCAGAUCCGCUGCC |
| | | | | GCUUCCCCAACUCCACCCGCACCGAGUUCCGC |
| | | | | CUGCAGAUCUGGCGCUACGCCACCGCCACCGA |
| | | | | CGCCGAGAUCGGCACCGCCCCCUCCCUGGAGG |
| | | | | AGGUGAUGGUGAACGUGUCCGCCCCCCCCGG |
| | | | | CGGCCAGCUGGUGUACGACUCCGCCCCCAACC |
| | | | | GCACCGACCCCCACGUGAUCUGGGCCGAGGG |
| | | | | CGCCGGCCCCGGCGCCUCCCCCCGCCUGUACU |
| | | | | CCGUGGUGGGCCCCCUGGGCCGCCAGCGCCUG |
| | | | | AUCAUCGAGGAGCUGACCCUGGAGACCCAGG |
| | | | | GCAUGUACUACUGGGUGUGGGGCCGCACCGA |
| | | | | CCGCCCCUCCGCCUACGGCACCUGGGUGCGCG |
| | | | | UGCGCGUGUUCCGCCCCCCCUCCCUGACCAUC |
| | | | | CACCCCCACGCCGUGCUGGAGGGCCAGCCCUU |
| | | | | CAAGGCCACCUGCACCGCCGCCACCUACUACC |
| | | | | CCGGCAACCGCGCCGAGUUCGUGUGGUUCGA |
| | | | | GGACGGCCGCCGCGUGUUCGACCCCGCCCAGA |
| | | | | UCCACACCCAGACCCAGGAGAACCCCGACGGC |
| | | | | UUCUCCACCGUGUCCACCGUGACCUCCGCCGC |
| | | | | CGUGGGCGGCCAGGGCCCCCCCCGCACCUUCA |
| | | | | CCUGCCAGCUGACCUGGCACCGCGACUCCGUG |
| | | | | UCCUUCUCCCGCCGCAACGCCUCCGGCACCGC |
| | | | | CUCCGUGCUGCCCCGCCCCACCAUCACCAUGG |
| | | | | AGUUCACCGGCGACCACGCCGUGUGCACCGCC |
| | | | | GGCUGCGUGCCCGAGGGCGUGACCUUCGCCU |
| | | | | GGUUCCUGGGCGACGACUCCUCCCCCGCCGAG |
| | | | | AAGGUGGCCGUGGCCUCCCAGACCUCCUGCG |
| | | | | GCCGCCCCGGCACCGCCACCAUCCGCUCCACC |
| | | | | CUGCCCGUGUCCUACGAGCAGACCGAGUACA |
| | | | | UCUGCCGCCUGGCCGGCUACCCCGACGGCAUC |
| | | | | CCCGUGCUGGAGCACCACUAA |
| 105 | IL2 | HSV-2 | Version | AUGAGAAUGCAGCUGCUGCUCCUGAUCGCCC |
| | | gC | 1 | UGUCUCUGGCCCUGGUCACCAAUAGCGCUUC |
| | | | | UCCCGGCAGAACCAUCACAGUGGGCCCUAGA |
| | | | | GGCAACGCCUCUAAUGCCGCUCCUAGCGCCUC |
| | | | | UCCUAGAAACGCCUCUGCUCCCAGAACCACAC |
| | | | | CUACACCUCCACAGCCUAGAAAGGCCACCAA |
| | | | | GAGCAAGGCCAGCACAGCCAAACCUGCUCCU |
| | | | | CCACCUAAGACAGGCCCUCCAAAGACAAGCU |
| | | | | CUGAGCCCGUGCGGUGCAACAGACACGAUCC |
| | | | | ACUGGCCAGAUACGGCAGCCGGGUGCAGAUC |
| | | | | AGAUGCAGAUUCCCCAACAGCACCCGGACCG |
| | | | | AGUUCCGGCUCCAGAUUUGGAGAUACGCCAC |
| | | | | CGCCACAGAUGCCGAGAUUGGAACAGCCCCU |
| | | | | AGCCUGGAAGAAGUGAUGGUCAACGUUUCAG |
| | | | | CCCCUCCUGGCGGCCAGCUGGUGUAUGAUUC |
| | | | | UGCCCCUAACCGGACCGAUCCUCACGUGAUA |
| | | | | UGGGCUGAAGGUGCUGGCCCAGGCGCAAGCC |
| | | | | CUAGACUGUAUUCUGUUGUGGGCCCUCUGGG |
| | | | | CAGACAGCGGCUGAUCAUUGAGGAACUGACC |
| | | | | CUGGAAACCCAGGGCAUGUACUACUGGGUCU |
| | | | | GGGGCAGAACCGAUAGACCAAGCGCCUAUGG |
| | | | | CACCUGGGUUCGAGUGCGAGUGUUCAGACCU |
| | | | | CCUAGCCUGACCAUCCAUCCUCACGCCGUUCU |
| | | | | GGAAGGCCAGCCUUUCAAGGCCACAUGUACC |
| | | | | GCCGCCACCUACUAUCCCGGAAACAGAGCCG |
| | | | | AGUUCGUUUGGUUCGAGGACGGCAGAAGGGU |
| | | | | GUUCGACCCCGCUCAGAUCCACACACAGACCC |
| | | | | AAGAGAACCCCGACGGCUUUAGCACCGUGUC |
| | | | | CACAGUGACAUCUGCCGCCGUUGGAGGACAG |
| | | | | GGCCCUCCUAGAACCUUUACCUGCCAGCUGA |
| | | | | CCUGGCACAGAGACAGCGUGUCCUUCAGCAG |
| | | | | AAGAAACGCCAGCGGCACAGCCAGCGUUCUG |
| | | | | CCUAGACCUACCAUCACCAUGGAAUUCACCG |
| | | | | GCGACCACGCCGUGUGUACAGCUGGAUGUGU |
| | | | | UCCUGAGGGCGUGACCUUCGCUUGGUUUCUG |
| | | | | GGCGACGAUAGCAGCCCUGCCGAAAAAGUGG |
| | | | | CUGUGGCCAGCCAGACAAGCUGUGGCAGACC |
| | | | | UGGAACCGCCACCAUCAGAAGCACACUGCCU |
| | | | | GUCAGCUACGAGCAGACCGAGUACAUCUGUC |
| | | | | GGCUGGCCGGCUAUCCUGAUGGCAUCCCUGU |
| | | | | GCUGGAACACCACUGA |
| 106 | IL2 | HSV-2 | Version | AUGCGCAUGCAACUGCUCCUGCUGAUUGCGU |
| | | gC | 2 | UGAGCCUUGCCCUGGUGACCAACAGCGCAAG |
| | | | | CCCCGGCAGAACCAUAACAGUAGGGCCACGG |
| | | | | GGGAAUGCUUCCAAUGCUGCACCUUCCGCUU |
| | | | | CACCGAGGAAUGCUUCUGCCCCAAGAACUAC |
| | | | | CCCCACUCCUCCUCAACCCAGGAAAGCGACAA |
| | | | | AGUCCAAGGCCAGCACCGCAAAACCCGCUCCU |
| | | | | CCUCCAAAGACUGGGCCCCCAAAGACAAGUA |
| | | | | GCGAACCAGUUCGGUGCAACAGGCAUGACCC |
| | | | | ACUUGCACGCUAUGGGUCAAGAGUCCAGAUA |
| | | | | CGGUGUCGCUUCCCUAACAGUACAAGGACUG |
| | | | | AGUUUCGGCUGCAGAUCUGGCGUUAUGCCAC |
| | | | | AGCUACUGACGCAGAGAUUGGUACCGCCCCC |
| | | | | AGUUUGGAAGAGGUGAUGGUCAACGUGUCCG |
| | | | | CACCCCCAGGAGGACAGCUGGUCUAUGACUC |
| | | | | AGCGCCCAAUAGGACCGAUCCCCACGUGAUC |
| | | | | UGGGCAGAAGGAGCCGGUCCUGGGGCCUCUC |
| | | | | CACGGCUGUACUCAGUUGUUGGCCCGCUUGG |
| | | | | ACGACAGAGACUCAUCAUCGAAGAGCUGACA |
| | | | | CUGGAGACACAGGGGAUGUACUACUGGGUGU |
| | | | | GGGGCCGUACUGACCGCCCUUCCGCAUAUGG |
| | | | | CACUUGGGUGAGAGUUCGCGUCUUUCGGCCC |
| | | | | CCUUCUCUCACCAUCCAUCCUCAUGCCGUGCU |
| | | | | CGAAGGCCAGCCCUUUAAGGCCACAUGCACU |
| | | | | GCUGCGACCUACUACCCUGGCAACAGAGCCG |
| | | | | AGUUUGUCUGGUUUGAGGAUGGUCGGCGAGU |
| | | | | AUUCGAUCCAGCCCAGAUUCACACACAAACG |
| | | | | CAGGAAAAUCCGGACGGCUUCAGCACAGUGU |
| | | | | CCACGGUGACCUCUGCUGCAGUUGGUGGACA |
| | | | | AGGACCCCCUCGAACCUUCACCUGUCAGCUG |
| | | | | ACCUGGCACAGAGACUCCGUAAGCUUCAGCC |
| | | | | GUAGAAACGCCUCUGGAACCGCCAGUGUGUU |
| | | | | GCCGAGGCCGACUAUCACGAUGGAAUUCACA |
| | | | | GGCGAUCAUGCCGUCUGUACUGCCGGCUGUG |
| | | | | UGCCAGAAGGCGUAACCUUCGCUUGGUUUCU |
| | | | | CGGGGAUGACUCAAGUCCUGCAGAGAAAGUG |
| | | | | GCUGUGGCCUCUCAGACGAGCUGCGGUCGAC |
| | | | | CAGGAACAGCUACCAUUCGCAGCACUCUGCC |
| | | | | CGUGUCCUACGAGCAGACGGAGUACAUCUGC |
| | | | | AGGCUGGCCGGCUAUCCCGAUGGGAUUCCAG |
| | | | | UCCUGGAGCACCACUGA |
| 107 | IL2 | HSV-2 | Version | AUGAGAAUGCAGCUGCUGCUGCUGAUCGCCC |
| | | gC | 3 | UGUCCCUGGCCCUGGUGACCAACUCCGCCUCC |
| | | | | CCCGGCAGAACCAUCACCGUGGGCCCCAGAG |
| | | | | GCAACGCCUCCAACGCCGCCCCCUCCGCCUCC |
| | | | | CCCAGAAACGCCUCCGCCCCCAGAACCACCCC |
| | | | | CACCCCUCCCCAGCCCAGAAAAGCCACCAAAU |
| | | | | CCAAAGCCUCCACCGCCAAACCCGCCCCUCCU |
| | | | | CCCAAAACCGGCCCUCCCAAAACCUCCUCCGA |
| | | | | ACCCGUGAGAUGCAACAGACACGAUCCCCUG |
| | | | | GCCAGAUACGGCUCCAGAGUGCAGAUCAGAU |
| | | | | GCAGAUUCCCCAACUCCACCAGAACCGAAUU |
| | | | | CAGACUGCAGAUCUGGAGAUACGCCACCGCC |
| | | | | ACCGAUGCCGAAAUCGGCACCGCCCCCUCCCU |
| | | | | GGAAGAAGUGAUGGUGAACGUGUCCGCCCCU |
| | | | | CCUGGCGGCCAGCUGGUGUACGAUUCCGCCC |
| | | | | CCAACAGAACCGAUCCCCACGUGAUCUGGGC |
| | | | | CGAAGGCGCCGGCCCCGGCGCCUCCCCCAGAC |
| | | | | UGUACUCCGUGGUGGGCCCCCUGGGCAGACA |
| | | | | GAGACUGAUCAUCGAAGAACUGACCCUGGAA |
| | | | | ACCCAGGGCAUGUACUACUGGGUGUGGGGCA |
| | | | | GAACCGAUAGACCCUCCGCCUACGGCACCUG |
| | | | | GGUGAGAGUGAGAGUGUUCAGACCCCCUUCC |
| | | | | CUGACCAUCCACCCCCACGCCGUGCUGGAAGG |
| | | | | CCAGCCCUUCAAAGCCACCUGCACCGCCGCCA |
| | | | | CCUACUACCCCGGCAACAGAGCCGAAUUCGU |
| | | | | GUGGUUCGAAGAUGGCAGAAGGGUGUUCGAU |
| | | | | CCCGCCCAGAUCCACACCCAGACCCAGGAAAA |
| | | | | CCCCGACGGCUUCUCCACCGUGUCCACCGUGA |
| | | | | CCUCCGCCGCCGUGGGCGGCCAGGGCCCUCCC |
| | | | | AGAACCUUCACCUGCCAGCUGACCUGGCACA |
| | | | | GAGACUCCGUGUCCUUCUCCAGAAGAAACGC |
| | | | | CUCCGGCACCGCCUCCGUGCUGCCCAGACCCA |
| | | | | CCAUCACCAUGGAAUUCACCGGCGAUCACGC |
| | | | | CGUGUGCACCGCCGGCUGCGUGCCCGAAGGC |
| | | | | GUGACCUUCGCCUGGUUCCUGGGCGAUGAUU |
| | | | | CCUCCCCCGCCGAAAAAGUGGCCGUGGCCUCC |
| | | | | CAGACCUCCUGCGGCAGACCCGGCACCGCCAC |
| | | | | CAUCAGAUCCACCCUGCCCGUGUCCUACGAAC |
| | | | | AGACCGAAUACAUCUGCAGACUGGCCGGCUA |
| | | | | CCCCGAUGGCAUCCCCGUGCUGGAACACCACU |
| | | | | GA |
| 108 | HSV-2 | HSV-2 | | AUGGGCCGCCUGACCUCCGGCGUGGGCACCG |
| | gD- | gC | | CCGCCCUGCUGGUGGUGGCCGUGGGCCUGCG |
| | KYA | | | CGUGGUGUGCGCCAAGUACGCCGCCUCCCCCG |
| | | | | GCCGCACCAUCACCGUGGGCCCCCGCGGCAAC |
| | | | | GCCUCCAACGCCGCCCCCUCCGCCUCCCCCCG |
| | | | | CAACGCCUCCGCCCCCCGCACCACCCCCACCC |
| | | | | CCCCCCAGCCCCGCAAGGCCACCAAGUCCAAG |
| | | | | GCCUCCACCGCCAAGCCCGCCCCCCCCCCCAA |
| | | | | GACCGGCCCCCCCAAGACCUCCUCCGAGCCCG |
| | | | | UGCGCUGCAACCGCCACGACCCCCUGGCCCGC |
| | | | | UACGGCUCCCGCGUGCAGAUCCGCUGCCGCU |
| | | | | UCCCCAACUCCACCCGCACCGAGUUCCGCCUG |
| | | | | CAGAUCUGGCGCUACGCCACCGCCACCGACGC |
| | | | | CGAGAUCGGCACCGCCCCCUCCCUGGAGGAG |
| | | | | GUGAUGGUGAACGUGUCCGCCCCCCCCGGCG |
| | | | | GCCAGCUGGUGUACGACUCCGCCCCCAACCGC |
| | | | | ACCGACCCCCACGUGAUCUGGGCCGAGGGCG |
| | | | | CCGGCCCCGGCGCCUCCCCCCGCCUGUACUCC |
| | | | | GUGGUGGGCCCCCUGGGCCGCCAGCGCCUGA |
| | | | | UCAUCGAGGAGCUGACCCUGGAGACCCAGGG |
| | | | | CAUGUACUACUGGGUGUGGGGCCGCACCGAC |
| | | | | CGCCCCUCCGCCUACGGCACCUGGGUGCGCGU |
| | | | | GCGCGUGUUCCGCCCCCCCUCCCUGACCAUCC |
| | | | | ACCCCCACGCCGUGCUGGAGGGCCAGCCCUUC |
| | | | | AAGGCCACCUGCACCGCCGCCACCUACUACCC |
| | | | | CGGCAACCGCGCCGAGUUCGUGUGGUUCGAG |
| | | | | GACGGCCGCCGCGUGUUCGACCCCGCCCAGAU |
| | | | | CCACACCCAGACCCAGGAGAACCCCGACGGCU |
| | | | | UCUCCACCGUGUCCACCGUGACCUCCGCCGCC |
| | | | | GUGGGCGGCCAGGGCCCCCCCCGCACCUUCAC |
| | | | | CUGCCAGCUGACCUGGCACCGCGACUCCGUG |
| | | | | UCCUUCUCCCGCCGCAACGCCUCCGGCACCGC |
| | | | | CUCCGUGCUGCCCCGCCCCACCAUCACCAUGG |
| | | | | AGUUCACCGGCGACCACGCCGUGUGCACCGCC |
| | | | | GGCUGCGUGCCCGAGGGCGUGACCUUCGCCU |
| | | | | GGUUCCUGGGCGACGACUCCUCCCCCGCCGAG |
| | | | | AAGGUGGCCGUGGCCUCCCAGACCUCCUGCG |
| | | | | GCCGCCCCGGCACCGCCACCAUCCGCUCCACC |
| | | | | CUGCCCGUGUCCUACGAGCAGACCGAGUACA |
| | | | | UCUGCCGCCUGGCCGGCUACCCCGACGGCAUC |
| | | | | CCCGUGCUGGAGCACCACUAA |
| 109 | HSV-2 | HSV-2 | Version | AUGGGGAGACUCACAUCAGGCGUAGGAACCG |
| | gD- | gC | 2 | CUGCCCUGUUGGUCGUGGCCGUUGGUCUGAG |
| | KYA | | | AGUUGUGUGUGCCAAAUAUGCUGCAAGCCCC |
| | | | | GGCAGAACCAUAACAGUAGGGCCACGGGGGA |
| | | | | AUGCUUCCAAUGCUGCACCUUCCGCUUCACC |
| | | | | GAGGAAUGCUUCUGCCCCAAGAACUACCCCC |
| | | | | ACUCCUCCUCAACCCAGGAAAGCGACAAAGU |
| | | | | CCAAGGCCAGCACCGCAAAACCCGCUCCUCCU |
| | | | | CCAAAGACUGGGCCCCCAAAGACAAGUAGCG |
| | | | | AACCAGUUCGGUGCAACAGGCAUGACCCACU |
| | | | | UGCACGCUAUGGGUCAAGAGUCCAGAUACGG |
| | | | | UGUCGCUUCCCUAACAGUACAAGGACUGAGU |
| | | | | UUCGGCUGCAGAUCUGGCGUUAUGCCACAGC |
| | | | | UACUGACGCAGAGAUUGGUACCGCCCCCAGU |
| | | | | UUGGAGGAAGUGAUGGUCAACGUGUCCGCAC |
| | | | | CCCCAGGAGGACAGCUGGUCUAUGACUCAGC |
| | | | | GCCCAAUAGGACCGAUCCCCACGUGAUCUGG |
| | | | | GCAGAAGGAGCCGGUCCUGGGGCCUCUCCAC |
| | | | | GGCUGUACUCAGUUGUUGGCCCGCUUGGACG |
| | | | | ACAGAGACUCAUCAUCGAGGAACUGACACUG |
| | | | | GAGACACAGGGGAUGUACUACUGGGUGUGGG |
| | | | | GCCGUACUGACCGCCCUUCCGCAUAUGGCAC |
| | | | | UUGGGUGAGAGUUCGCGUCUUUCGGCCCCCU |
| | | | | UCUCUCACCAUCCAUCCUCAUGCCGUGCUCGA |
| | | | | AGGCCAGCCCUUUAAGGCCACAUGCACUGCU |
| | | | | GCGACCUACUACCCUGGCAACAGAGCCGAGU |
| | | | | UUGUCUGGUUUGAGGAUGGUCGGCGAGUAUU |
| | | | | CGAUCCAGCCCAGAUUCACACACAAACGCAG |
| | | | | GAAAAUCCGGACGGCUUCAGCACAGUGUCCA |
| | | | | CGGUGACCUCUGCUGCAGUUGGUGGACAAGG |
| | | | | ACCCCCUCGAACCUUCACCUGUCAGCUGACCU |
| | | | | GGCACAGAGACUCCGUAAGCUUCAGCCGUAG |
| | | | | AAACGCCUCUGGAACCGCCAGUGUGUUGCCG |
| | | | | AGGCCGACUAUCACGAUGGAAUUCACAGGCG |
| | | | | AUCAUGCCGUCUGUACUGCCGGCUGUGUGCC |
| | | | | AGAAGGCGUAACCUUCGCUUGGUUUCUCGGG |
| | | | | GAUGACUCAAGUCCUGCAGAGAAAGUGGCUG |
| | | | | UGGCCUCUCAGACGAGCUGCGGUCGACCAGG |
| | | | | AACAGCUACCAUUCGCAGCACUCUGCCCGUG |
| | | | | UCCUACGAGCAGACGGAGUACAUCUGCAGGC |
| | | | | UGGCCGGCUAUCCCGAUGGGAUUCCAGUCCU |
| | | | | GGAGCACCACUGA |
| 110 | HSV-2 | HSV-2 | Version | AUGGGGAGACUCACAUCAGGCGUAGGAACCG |
| | gD- | gC | 2.1 | CUGCCCUGUUGGUCGUGGCCGUUGGUCUGAG |
| | KYA | | | AGUUGUGUGUGCCAAAUAUGCUGCAAGCCCC |
| | | | | GGCAGAACCAUAACAGUAGGGCCACGGGGGA |
| | | | | AUGCUUCCAAUGCUGCACCUUCCGCUUCACC |
| | | | | GAGGAAUGCUUCUGCCCCAAGAACUACCCCC |
| | | | | ACUCCUCCUCAACCCAGGAAAGCGACAAAGU |
| | | | | CCAAGGCCAGCACCGCAAAACCCGCUCCUCCU |
| | | | | CCAAAGACUGGGCCCCCAAAGACAAGUAGCG |
| | | | | AACCAGUUCGGUGCAACAGGCAUGACCCACU |
| | | | | UGCACGCUAUGGGUCAAGAGUCCAGAUACGG |
| | | | | UGUCGCUUCCCUAACAGUACAAGGACUGAGU |
| | | | | UUCGGCUGCAGAUCUGGCGUUAUGCCACAGC |
| | | | | UACUGACGCAGAGAUUGGUACCGCCCCCAGU |
| | | | | UUGGAAGAGGUGAUGGUCAACGUGUCCGCAC |
| | | | | CCCCAGGAGGACAGCUGGUCUAUGACUCAGC |
| | | | | GCCCAAUAGGACCGAUCCCCACGUGAUCUGG |
| | | | | GCAGAAGGAGCCGGUCCUGGGGCCUCUCCAC |
| | | | | GGCUGUACUCAGUUGUUGGCCCGCUUGGACG |
| | | | | ACAGAGACUCAUCAUCGAAGAGCUGACACUG |
| | | | | GAGACACAGGGGAUGUACUACUGGGUGUGGG |
| | | | | GCCGUACUGACCGCCCUUCCGCAUAUGGCAC |
| | | | | UUGGGUGAGAGUUCGCGUCUUUCGGCCCCCU |
| | | | | UCUCUCACCAUCCAUCCUCAUGCCGUGCUCGA |
| | | | | AGGCCAGCCCUUUAAGGCCACAUGCACUGCU |
| | | | | GCGACCUACUACCCUGGCAACAGAGCCGAGU |
| | | | | UUGUCUGGUUUGAGGAUGGUCGGCGAGUAUU |
| | | | | CGAUCCAGCCCAGAUUCACACACAAACGCAG |
| | | | | GAAAAUCCGGACGGCUUCAGCACAGUGUCCA |
| | | | | CGGUGACCUCUGCUGCAGUUGGUGGACAAGG |
| | | | | ACCCCCUCGAACCUUCACCUGUCAGCUGACCU |
| | | | | GGCACAGAGACUCCGUAAGCUUCAGCCGUAG |
| | | | | AAACGCCUCUGGAACCGCCAGUGUGUUGCCG |
| | | | | AGGCCGACUAUCACGAUGGAAUUCACAGGCG |
| | | | | AUCAUGCCGUCUGUACUGCCGGCUGUGUGCC |
| | | | | AGAAGGCGUAACCUUCGCUUGGUUUCUCGGG |
| | | | | GAUGACUCAAGUCCUGCAGAGAAAGUGGCUG |
| | | | | UGGCCUCUCAGACGAGCUGCGGUCGACCAGG |
| | | | | AACAGCUACCAUUCGCAGCACUCUGCCCGUG |
| | | | | UCCUACGAGCAGACGGAGUACAUCUGCAGGC |
| | | | | UGGCCGGCUAUCCCGAUGGGAUUCCAGUCCU |
| | | | | GGAGCACCACUGA |
| 111 | HSV-2 | HSV-2 | Version | AUGGGGAGACUCACAUCAGGCGUAGGAACCG |
| | gD- | gC | 2.2 | CUGCCCUGUUGGUCGUGGCCGUUGGUCUGAG |
| | KYA | | | AGUUGUGUGUGCCAAAUAUGCUGCAAGCCCC |
| | | | | GGCAGAACCAUAACAGUAGGGCCACGGGGGA |
| | | | | AUGCUUCCAAUGCUGCACCUUCCGCUUCACC |
| | | | | GAGGAAUGCUUCUGCCCCAAGAACUACCCCC |
| | | | | ACUCCUCCUCAACCCAGGAAAGCGACAAAGU |
| | | | | CCAAGGCCAGCACCGCAAAACCCGCUCCUCCU |
| | | | | CCAAAGACUGGGCCCCCAAAGACAAGUAGCG |
| | | | | AACCAGUUCGGUGCAACAGGCAUGACCCACU |
| | | | | UGCACGCUAUGGGUCAAGAGUCCAGAUACGG |
| | | | | UGUCGCUUCCCUAACAGUACAAGGACUGAGU |
| | | | | UUCGGCUGCAGAUCUGGCGUUAUGCCACAGC |
| | | | | UACUGACGCAGAGAUUGGUACCGCCCCCAGU |
| | | | | UUGGAAGAGGUGAUGGUCAACGUGUCCGCAC |
| | | | | CCCCAGGAGGACAGCUGGUCUAUGACUCAGC |
| | | | | GCCCAAUAGGACCGAUCCCCACGUGAUCUGG |
| | | | | GCAGAAGGAGCCGGUCCUGGGGCCUCUCCAC |
| | | | | GGCUGUACUCAGUUGUUGGCCCGCUUGGACG |
| | | | | ACAGAGACUCAUCAUCGAAGAGCUGACACUG |
| | | | | GAGACACAGGGGAUGUACUACUGGGUGUGGG |
| | | | | GCCGUACUGACCGCCCUUCCGCAUAUGGCAC |
| | | | | UUGGGUGAGAGUUCGCGUCUUUCGGCCCCCU |
| | | | | UCUCUCACCAUCCAUCCUCAUGCCGUGCUCGA |
| | | | | AGGCCAGCCCUUUAAGGCCACAUGCACUGCU |
| | | | | GCGACCUACUACCCUGGCAACAGAGCCGAGU |
| | | | | UUGUCUGGUUUGAGGAUGGUCGGCGAGUAUU |
| | | | | CGAUCCAGCCCAGAUUCACACACAAACGCAG |
| | | | | GAAAAUCCGGACGGCUUCAGCACAGUGUCCA |
| | | | | CGGUGACCUCUGCUGCAGUUGGUGGACAAGG |
| | | | | ACCCCCUCGAACCUUCACCUGUCAGCUGACCU |
| | | | | GGCACAGAGACUCCGUAAGCUUCAGCCGUAG |
| | | | | AAACGCCUCUGGAACCGCCAGUGUGUUGCCG |
| | | | | AGGCCGACUAUCACGAUGGAAUUCACAGGCG |
| | | | | AUCAUGCCGUCUGUACUGCCGGCUGUGUGCC |
| | | | | AGAAGGCGUAACCUUCGCUUGGUUUCUCGGG |
| | | | | GAUGACUCAAGUCCUGCAGAGAAAGUGGCUG |
| | | | | UGGCCUCUCAGACGAGCUGCGGUCGACCAGG |
| | | | | AACAGCUACCAUUCGCAGCACUCUGCCCGUG |
| | | | | UCCUACGAGCAGACGGAGUACAUCUGCAGGC |
| | | | | UGGCCGGCUAUCCCGAUGGGAUUCCAGUCCU |
| | | | | GGAGCACCACUGA |
| 112 | HSV-2 | HSV-2 | Version | AUGGGCAGACUGACAUCUGGCGUGGGAACAG |
| | gD- | gC | 1 | CUGCUCUGCUGGUGGUUGCUGUGGGCCUGAG |
| | KYALA | | | AGUCGUGUGUGCCAAAUACGCCCUGGCCUCU |
| | | | | CCCGGCAGAACCAUCACAGUGGGCCCUAGAG |
| | | | | GCAACGCCUCUAAUGCCGCUCCUAGCGCCUCU |
| | | | | CCUAGAAACGCCUCUGCUCCCAGAACCACACC |
| | | | | UACACCUCCACAGCCUAGAAAGGCCACCAAG |
| | | | | AGCAAGGCCAGCACAGCCAAACCUGCUCCUCC |
| | | | | ACCUAAGACAGGCCCUCCAAAGACAAGCUCU |
| | | | | GAGCCCGUGCGGUGCAACAGACACGAUCCAC |
| | | | | UGGCCAGAUACGGCAGCCGGGUGCAGAUCAG |
| | | | | AUGCAGAUUCCCCAACAGCACCCGGACCGAG |
| | | | | UUCCGGCUCCAGAUUUGGAGAUACGCCACCG |
| | | | | CCACAGAUGCCGAGAUUGGAACAGCCCCUAG |
| | | | | CCUGGAAGAAGUGAUGGUCAACGUUUCAGCC |
| | | | | CCUCCUGGCGGCCAGCUGGUGUAUGAUUCUG |
| | | | | CCCCUAACCGGACCGAUCCUCACGUGAUAUG |
| | | | | GGCUGAAGGUGCUGGCCCAGGCGCAAGCCCU |
| | | | | AGACUGUAUUCUGUUGUGGGCCCUCUGGGCA |
| | | | | GACAGCGGCUGAUCAUUGAGGAACUGACCCU |
| | | | | GGAAACCCAGGGCAUGUACUACUGGGUCUGG |
| | | | | GGCAGAACCGAUAGACCAAGCGCCUAUGGCA |
| | | | | CCUGGGUUCGAGUGCGAGUGUUCAGACCUCC |
| | | | | UAGCCUGACCAUCCAUCCUCACGCCGUUCUG |
| | | | | GAAGGCCAGCCUUUCAAGGCCACAUGUACCG |
| | | | | CCGCCACCUACUAUCCCGGAAACAGAGCCGA |
| | | | | GUUCGUUUGGUUCGAGGACGGCAGAAGGGUG |
| | | | | UUCGACCCCGCUCAGAUCCACACACAGACCCA |
| | | | | AGAGAACCCCGACGGCUUUAGCACCGUGUCC |
| | | | | ACAGUGACAUCUGCCGCCGUUGGAGGACAGG |
| | | | | GCCCUCCUAGAACCUUUACCUGCCAGCUGACC |
| | | | | UGGCACAGAGACAGCGUGUCCUUCAGCAGAA |
| | | | | GAAACGCCAGCGGCACAGCCAGCGUUCUGCC |
| | | | | UAGACCUACCAUCACCAUGGAAUUCACCGGC |
| | | | | GACCACGCCGUGUGUACAGCUGGAUGUGUUC |
| | | | | CUGAGGGCGUGACCUUCGCUUGGUUUCUGGG |
| | | | | CGACGAUAGCAGCCCUGCCGAAAAAGUGGCU |
| | | | | GUGGCCAGCCAGACAAGCUGUGGCAGACCUG |
| | | | | GAACCGCCACCAUCAGAAGCACACUGCCUGU |
| | | | | CAGCUACGAGCAGACCGAGUACAUCUGUCGG |
| | | | | CUGGCCGGCUAUCCUGAUGGCAUCCCUGUGC |
| | | | | UGGAACACCACUGA |
| 113 | HSV-2 | HSV-2 | Version | AUGGGGAGACUCACAUCAGGCGUAGGAACCG |
| | gD- | gC | 2 | CUGCCCUGUUGGUCGUGGCCGUUGGUCUGAG |
| | KYALA | | | AGUUGUGUGUGCCAAAUAUGCUCUGGCAAGC |
| | | | | CCCGGCAGAACCAUAACAGUAGGGCCACGGG |
| | | | | GGAAUGCUUCCAAUGCUGCACCUUCCGCUUC |
| | | | | ACCGAGGAAUGCUUCUGCCCCAAGAACUACC |
| | | | | CCCACUCCUCCUCAACCCAGGAAAGCGACAAA |
| | | | | GUCCAAGGCCAGCACCGCAAAACCCGCUCCUC |
| | | | | CUCCAAAGACUGGGCCCCCAAAGACAAGUAG |
| | | | | CGAACCAGUUCGGUGCAACAGGCAUGACCCA |
| | | | | CUUGCACGCUAUGGGUCAAGAGUCCAGAUAC |
| | | | | GGUGUCGCUUCCCUAACAGUACAAGGACUGA |
| | | | | GUUUCGGCUGCAGAUCUGGCGUUAUGCCACA |
| | | | | GCUACUGACGCAGAGAUUGGUACCGCCCCCA |
| | | | | GUUUGGAGGAAGUGAUGGUCAACGUGUCCGC |
| | | | | ACCCCCAGGAGGACAGCUGGUCUAUGACUCA |
| | | | | GCGCCCAAUAGGACCGAUCCCCACGUGAUCU |
| | | | | GGGCAGAAGGAGCCGGUCCUGGGGCCUCUCC |
| | | | | ACGGCUGUACUCAGUUGUUGGCCCGCUUGGA |
| | | | | CGACAGAGACUCAUCAUCGAGGAACUGACAC |
| | | | | UGGAGACACAGGGGAUGUACUACUGGGUGUG |
| | | | | GGGCCGUACUGACCGCCCUUCCGCAUAUGGC |
| | | | | ACUUGGGUGAGAGUUCGCGUCUUUCGGCCCC |
| | | | | CUUCUCUCACCAUCCAUCCUCAUGCCGUGCUC |
| | | | | GAAGGCCAGCCCUUUAAGGCCACAUGCACUG |
| | | | | CUGCGACCUACUACCCUGGCAACAGAGCCGA |
| | | | | GUUUGUCUGGUUUGAGGAUGGUCGGCGAGUA |
| | | | | UUCGAUCCAGCCCAGAUUCACACACAAACGC |
| | | | | AGGAAAAUCCGGACGGCUUCAGCACAGUGUC |
| | | | | CACGGUGACCUCUGCUGCAGUUGGUGGACAA |
| | | | | GGACCCCCUCGAACCUUCACCUGUCAGCUGAC |
| | | | | CUGGCACAGAGACUCCGUAAGCUUCAGCCGU |
| | | | | AGAAACGCCUCUGGAACCGCCAGUGUGUUGC |
| | | | | CGAGGCCGACUAUCACGAUGGAAUUCACAGG |
| | | | | CGAUCAUGCCGUCUGUACUGCCGGCUGUGUG |
| | | | | CCAGAAGGCGUAACCUUCGCUUGGUUUCUCG |
| | | | | GGGAUGACUCAAGUCCUGCAGAGAAAGUGGC |
| | | | | UGUGGCCUCUCAGACGAGCUGCGGUCGACCA |
| | | | | GGAACAGCUACCAUUCGCAGCACUCUGCCCG |
| | | | | UGUCCUACGAGCAGACGGAGUACAUCUGCAG |
| | | | | GCUGGCCGGCUAUCCCGAUGGGAUUCCAGUC |
| | | | | CUGGAGCACCACUGA |
| 318 | HSV-2 | HSV-2 | Version | AUGGGGAGACUCACAUCCGGAGUGGGCACUG |
| | gD- | gC | 2.1 | CUGCCCUGUUGGUCGUGGCCGUUGGUCUGAG |
| | KYALA | | | AGUUGUGUGUGCCAAAUAUGCUCUGGCAAGC |
| | | | | CCCGGCAGAACCAUAACAGUAGGGCCACGGG |
| | | | | GGAAUGCUUCCAAUGCUGCACCUUCCGCUUC |
| | | | | ACCGAGGAAUGCUUCUGCCCCAAGAACUACC |
| | | | | CCCACUCCUCCUCAACCCAGGAAAGCGACAAA |
| | | | | GUCCAAGGCCAGCACCGCAAAACCCGCUCCUC |
| | | | | CUCCAAAGACUGGGCCCCCAAAGACAAGUAG |
| | | | | CGAACCAGUUCGGUGCAACAGGCAUGACCCA |
| | | | | CUUGCACGCUAUGGGUCAAGAGUCCAGAUAC |
| | | | | GGUGUCGCUUCCCUAACAGUACAAGGACUGA |
| | | | | GUUUCGGCUGCAGAUCUGGCGUUAUGCCACA |
| | | | | GCUACUGACGCAGAGAUUGGUACCGCCCCCA |
| | | | | GUUUGGAGGAAGUGAUGGUCAACGUGUCCGC |
| | | | | ACCCCCAGGAGGACAGCUGGUCUAUGACUCA |
| | | | | GCGCCCAAUAGGACAGACCCACAUGUUAUCU |
| | | | | GGGCAGAAGGAGCCGGUCCUGGGGCCUCUCC |
| | | | | ACGGCUGUACUCAGUUGUUGGCCCGCUUGGA |
| | | | | CGACAGAGACUCAUCAUCGAGGAACUGACAC |
| | | | | UGGAGACACAGGGGAUGUACUACUGGGUGUG |
| | | | | GGGCCGUACUGACCGCCCUUCCGCAUAUGGC |
| | | | | ACUUGGGUGAGAGUUCGCGUCUUUCGGCCCC |
| | | | | CUUCUCUCACCAUCCAUCCUCAUGCCGUGCUC |
| | | | | GAAGGCCAGCCCUUUAAGGCCACAUGCACUG |
| | | | | CUGCGACCUACUACCCUGGCAACAGAGCCGA |
| | | | | GUUUGUCUGGUUUGAGGAUGGUCGGCGAGUA |
| | | | | UUCGAUCCAGCCCAGAUUCACACACAAACGC |
| | | | | AGGAAAAUCCGGACGGCUUCAGCACAGUGUC |
| | | | | CACGGUGACCUCUGCUGCAGUUGGUGGACAA |
| | | | | GGACCCCCUCGAACCUUCACCUGUCAGCUGAC |
| | | | | CUGGCACAGAGACUCCGUAAGCUUCAGCCGU |
| | | | | AGAAACGCCUCUGGAACCGCCAGUGUGUUGC |
| | | | | CGAGGCCGACUAUCACGAUGGAAUUCACAGG |
| | | | | CGAUCAUGCCGUCUGUACUGCCGGCUGUGUG |
| | | | | CCAGAAGGCGUAACCUUCGCUUGGUUUCUCG |
| | | | | GGGAUGACUCAAGUCCUGCAGAGAAAGUGGC |
| | | | | UGUGGCCUCUCAGACGAGCUGCGGUCGACCA |
| | | | | GGAACAGCUACCAUUCGCAGCACUCUGCCCG |
| | | | | UGUCCUACGAGCAGACGGAGUACAUCUGCAG |
| | | | | GCUGGCCGGCUAUCCCGAUGGGAUUCCAGUC |
| | | | | CUGGAGCACCACUGA |
| 114 | HSV-2 | HSV-2 | Version | AUGGGCAGACUGACCUCCGGCGUGGGCACCG |
| | gD- | gC | 3 | CCGCCCUGCUGGUGGUGGCCGUGGGCCUGAG |
| | KYALA | | | AGUGGUGUGCGCCAAAUACGCCCUGGCCUCC |
| | | | | CCCGGCAGAACCAUCACCGUGGGCCCCAGAG |
| | | | | GCAACGCCUCCAACGCCGCCCCCUCCGCCUCC |
| | | | | CCCAGAAACGCCUCCGCCCCCAGAACCACCCC |
| | | | | CACCCCUCCCCAGCCCAGAAAAGCCACCAAAU |
| | | | | CCAAAGCCUCCACCGCCAAACCCGCCCCUCCU |
| | | | | CCCAAAACCGGCCCUCCCAAAACCUCCUCCGA |
| | | | | ACCCGUGAGAUGCAACAGACACGAUCCCCUG |
| | | | | GCCAGAUACGGCUCCAGAGUGCAGAUCAGAU |
| | | | | GCAGAUUCCCCAACUCCACCAGAACCGAAUU |
| | | | | CAGACUGCAGAUCUGGAGAUACGCCACCGCC |
| | | | | ACCGAUGCCGAAAUCGGCACCGCCCCCUCCCU |
| | | | | GGAAGAAGUGAUGGUGAACGUGUCCGCCCCU |
| | | | | CCUGGCGGCCAGCUGGUGUACGAUUCCGCCC |
| | | | | CCAACAGAACCGAUCCCCACGUGAUCUGGGC |
| | | | | CGAAGGCGCCGGCCCCGGCGCCUCCCCCAGAC |
| | | | | UGUACUCCGUGGUGGGCCCCCUGGGCAGACA |
| | | | | GAGACUGAUCAUCGAAGAACUGACCCUGGAA |
| | | | | ACCCAGGGCAUGUACUACUGGGUGUGGGGCA |
| | | | | GAACCGAUAGACCCUCCGCCUACGGCACCUG |
| | | | | GGUGAGAGUGAGAGUGUUCAGACCCCCUUCC |
| | | | | CUGACCAUCCACCCCCACGCCGUGCUGGAAGG |
| | | | | CCAGCCCUUCAAAGCCACCUGCACCGCCGCCA |
| | | | | CCUACUACCCCGGCAACAGAGCCGAAUUCGU |
| | | | | GUGGUUCGAAGAUGGCAGAAGGGUGUUCGAU |
| | | | | CCCGCCCAGAUCCACACCCAGACCCAGGAAAA |
| | | | | CCCCGACGGCUUCUCCACCGUGUCCACCGUGA |
| | | | | CCUCCGCCGCCGUGGGCGGCCAGGGCCCUCCC |
| | | | | AGAACCUUCACCUGCCAGCUGACCUGGCACA |
| | | | | GAGACUCCGUGUCCUUCUCCAGAAGAAACGC |
| | | | | CUCCGGCACCGCCUCCGUGCUGCCCAGACCCA |
| | | | | CCAUCACCAUGGAAUUCACCGGCGAUCACGC |
| | | | | CGUGUGCACCGCCGGCUGCGUGCCCGAAGGC |
| | | | | GUGACCUUCGCCUGGUUCCUGGGCGAUGAUU |
| | | | | CCUCCCCCGCCGAAAAAGUGGCCGUGGCCUCC |
| | | | | CAGACCUCCUGCGGCAGACCCGGCACCGCCAC |
| | | | | CAUCAGAUCCACCCUGCCCGUGUCCUACGAAC |
| | | | | AGACCGAAUACAUCUGCAGACUGGCCGGCUA |
| | | | | CCCCGAUGGCAUCCCCGUGCUGGAACACCACU |
| | | | | GA |
| 115 | HSV-2 | HSV-2 | Version | AUGGCCUUGGGGAGAGUGGGCCUUGCAGUGG |
| | gC | gC | 2 | GUCUGUGGGGACUGCUCUGGGUUGGCGUAGU |
| | | | | CGUCGUGCUGGCUAACGCAAGCCCCGGCAGA |
| | | | | ACCAUAACAGUAGGGCCACGGGGGAAUGCUU |
| | | | | CCAAUGCUGCACCUUCCGCUUCACCGAGGAA |
| | | | | UGCUUCUGCCCCAAGAACUACCCCCACUCCUC |
| | | | | CUCAACCCAGGAAAGCGACAAAGUCCAAGGC |
| | | | | CAGCACCGCAAAACCCGCUCCUCCUCCAAAGA |
| | | | | CUGGGCCCCCAAAGACAAGUAGCGAACCAGU |
| | | | | UCGGUGCAACAGGCAUGACCCACUUGCACGC |
| | | | | UAUGGGUCAAGAGUCCAGAUACGGUGUCGCU |
| | | | | UCCCUAACAGUACAAGGACUGAGUUUCGGCU |
| | | | | GCAGAUCUGGCGUUAUGCCACAGCUACUGAC |
| | | | | GCAGAGAUUGGUACCGCCCCCAGUUUGGAGG |
| | | | | AAGUGAUGGUCAACGUGUCCGCACCCCCAGG |
| | | | | AGGACAGCUGGUCUAUGACUCAGCGCCCAAU |
| | | | | AGGACCGAUCCCCACGUGAUCUGGGCAGAAG |
| | | | | GAGCCGGUCCUGGGGCCUCUCCACGGCUGUA |
| | | | | CUCAGUUGUUGGCCCGCUUGGACGACAGAGA |
| | | | | CUCAUCAUCGAGGAACUGACACUGGAGACAC |
| | | | | AGGGGAUGUACUACUGGGUGUGGGGCCGUAC |
| | | | | UGACCGCCCUUCCGCAUAUGGCACUUGGGUG |
| | | | | AGAGUUCGCGUCUUUCGGCCCCCUUCUCUCA |
| | | | | CCAUCCAUCCUCAUGCCGUGCUCGAAGGCCA |
| | | | | GCCCUUUAAGGCCACAUGCACUGCUGCGACC |
| | | | | UACUACCCUGGCAACAGAGCCGAGUUUGUCU |
| | | | | GGUUUGAGGAUGGUCGGCGAGUAUUCGAUCC |
| | | | | AGCCCAGAUUCACACACAAACGCAGGAAAAU |
| | | | | CCGGACGGCUUCAGCACAGUGUCCACGGUGA |
| | | | | CCUCUGCUGCAGUUGGUGGACAAGGACCCCC |
| | | | | UCGAACCUUCACCUGUCAGCUGACCUGGCAC |
| | | | | AGAGACUCCGUAAGCUUCAGCCGUAGAAACG |
| | | | | CCUCUGGAACCGCCAGUGUGUUGCCGAGGCC |
| | | | | GACUAUCACGAUGGAAUUCACAGGCGAUCAU |
| | | | | GCCGUCUGUACUGCCGGCUGUGUGCCAGAAG |
| | | | | GCGUAACCUUCGCUUGGUUUCUCGGGGAUGA |
| | | | | CUCAAGUCCUGCAGAGAAAGUGGCUGUGGCC |
| | | | | UCUCAGACGAGCUGCGGUCGACCAGGAACAG |
| | | | | CUACCAUUCGCAGCACUCUGCCCGUGUCCUAC |
| | | | | GAGCAGACGGAGUACAUCUGCAGGCUGGCCG |
| | | | | GCUAUCCCGAUGGGAUUCCAGUCCUGGAGCA |
| | | | | CCACUGA |
| 319 | HSV-2 | HSV-2 | Version | AUGGCACUAGGGAGAGUGGGAUUAGCUGUGG |
| | gC | gC | 2.1 | GUCUGUGGGGACUGCUCUGGGUAGGAGUCGU |
| | | | | CGUCGUGCUGGCUAACGCAAGCCCCGGCAGA |
| | | | | ACCAUAACAGUAGGGCCACGGGGGAAUGCUU |
| | | | | CCAAUGCUGCACCUUCCGCUUCACCGAGGAA |
| | | | | UGCUUCUGCCCCAAGAACUACCCCCACUCCUC |
| | | | | CUCAACCCAGGAAAGCGACAAAGUCCAAGGC |
| | | | | CAGCACCGCAAAACCCGCUCCUCCUCCAAAGA |
| | | | | CUGGGCCCCCAAAGACAAGUAGCGAACCAGU |
| | | | | UCGGUGCAACAGGCAUGACCCACUUGCACGC |
| | | | | UAUGGGUCAAGAGUCCAGAUACGGUGUCGCU |
| | | | | UCCCUAACAGUACAAGGACUGAGUUUCGGCU |
| | | | | GCAGAUCUGGCGUUAUGCCACAGCUACUGAC |
| | | | | GCAGAGAUUGGUACCGCCCCCAGUUUGGAGG |
| | | | | AAGUGAUGGUCAACGUGUCCGCACCCCCAGG |
| | | | | AGGACAGCUGGUCUAUGACUCAGCGCCCAAU |
| | | | | AGGACCGAUCCCCACGUGAUCUGGGCAGAAG |
| | | | | GAGCCGGUCCUGGGGCCUCUCCACGGCUGUA |
| | | | | CUCAGUUGUUGGCCCGCUUGGACGACAGAGA |
| | | | | CUCAUCAUCGAGGAACUGACACUGGAGACAC |
| | | | | AGGGGAUGUACUACUGGGUGUGGGGCCGUAC |
| | | | | UGACCGCCCUUCCGCAUAUGGCACUUGGGUG |
| | | | | AGAGUUCGCGUCUUUCGGCCCCCUUCUCUCA |
| | | | | CCAUCCAUCCUCAUGCCGUGCUCGAAGGCCA |
| | | | | GCCCUUUAAGGCCACAUGCACUGCUGCGACC |
| | | | | UACUACCCUGGCAACAGAGCCGAGUUUGUCU |
| | | | | GGUUUGAGGAUGGUCGGCGAGUAUUCGAUCC |
| | | | | AGCCCAGAUUCACACACAAACGCAGGAAAAU |
| | | | | CCGGACGGCUUCAGCACAGUGUCCACGGUGA |
| | | | | CCUCUGCUGCAGUUGGUGGACAAGGACCCCC |
| | | | | UCGAACCUUCACCUGUCAGCUGACCUGGCAC |
| | | | | AGAGACUCCGUAAGCUUCAGCCGUAGAAACG |
| | | | | CCUCUGGAACCGCCAGUGUGUUGCCGAGGCC |
| | | | | GACUAUCACGAUGGAAUUCACAGGCGAUCAU |
| | | | | GCCGUCUGUACUGCCGGCUGUGUGCCAGAAG |
| | | | | GCGUAACCUUCGCUUGGUUUCUCGGGGAUGA |
| | | | | CUCAAGUCCUGCAGAGAAAGUGGCUGUGGCC |
| | | | | UCUCAGACGAGCUGCGGUCGACCAGGAACAG |
| | | | | CUACCAUUCGCAGCACUCUGCCCGUGUCCUAC |
| | | | | GAGCAGACGGAGUACAUCUGCAGGCUGGCCG |
| | | | | GCUAUCCCGAUGGGAUUCCAGUCCUGGAGCA |
| | | | | CCACUGA |
| 320 | HSV-2 | HSV-2 | Version | AUGGCCUUGGGGAGAGUCGGCCUUGCAGUGG |
| | gC | gC | 2.2 | GACUGUGGGGUCUGCUGUGGGUUGGCGUGGU |
| | | | | AGUCGUGCUCGCUAACGCCAGCCCCGGCAGA |
| | | | | ACCAUAACAGUAGGGCCACGGGGGAAUGCUU |
| | | | | CCAAUGCUGCACCUUCCGCUUCACCGAGGAA |
| | | | | UGCUUCUGCCCCAAGAACUACCCCCACUCCUC |
| | | | | CUCAACCCAGGAAAGCGACAAAGUCCAAGGC |
| | | | | CAGCACCGCAAAACCCGCUCCUCCUCCAAAGA |
| | | | | CUGGGCCCCCAAAGACAAGUAGCGAACCAGU |
| | | | | UCGGUGCAACAGGCAUGACCCACUUGCACGC |
| | | | | UAUGGGUCAAGAGUCCAGAUACGGUGUCGCU |
| | | | | UCCCUAACAGUACAAGGACUGAGUUUCGGCU |
| | | | | GCAGAUCUGGCGUUAUGCCACAGCUACUGAC |
| | | | | GCAGAGAUUGGUACCGCCCCCAGUUUGGAGG |
| | | | | AAGUGAUGGUCAACGUGUCCGCACCCCCAGG |
| | | | | AGGACAGCUGGUCUAUGACUCAGCGCCCAAU |
| | | | | AGGACCGAUCCCCACGUGAUCUGGGCAGAAG |
| | | | | GAGCCGGUCCUGGGGCCUCUCCACGGCUGUA |
| | | | | CUCAGUUGUUGGCCCGCUUGGACGACAGAGA |
| | | | | CUCAUCAUCGAGGAACUGACACUGGAGACAC |
| | | | | AGGGGAUGUACUACUGGGUGUGGGGCCGUAC |
| | | | | UGACCGCCCUUCCGCAUAUGGCACUUGGGUG |
| | | | | AGAGUUCGCGUCUUUCGGCCCCCUUCUCUCA |
| | | | | CCAUCCAUCCUCAUGCCGUGCUCGAAGGCCA |
| | | | | GCCCUUUAAGGCCACAUGCACUGCUGCGACC |
| | | | | UACUACCCUGGCAACAGAGCCGAGUUUGUCU |
| | | | | GGUUUGAGGAUGGUCGGCGAGUAUUCGAUCC |
| | | | | AGCCCAGAUUCACACACAAACGCAGGAAAAU |
| | | | | CCGGACGGCUUCAGCACAGUGUCCACGGUGA |
| | | | | CCUCUGCUGCAGUUGGUGGACAAGGACCCCC |
| | | | | UCGAACCUUCACCUGUCAGCUGACCUGGCAC |
| | | | | AGAGACUCCGUAAGCUUCAGCCGUAGAAACG |
| | | | | CCUCUGGAACCGCCAGUGUGUUGCCGAGGCC |
| | | | | GACUAUCACGAUGGAAUUCACAGGCGAUCAU |
| | | | | GCCGUCUGUACUGCCGGCUGUGUGCCAGAAG |
| | | | | GCGUAACCUUCGCUUGGUUUCUCGGGGAUGA |
| | | | | CUCAAGUCCUGCAGAGAAAGUGGCUGUGGCC |
| | | | | UCUCAGACGAGCUGCGGUCGACCAGGAACAG |
| | | | | CUACCAUUCGCAGCACUCUGCCCGUGUCCUAC |
| | | | | GAGCAGACGGAGUACAUCUGCAGGCUGGCCG |
| | | | | GCUAUCCCGAUGGGAUUCCAGUCCUGGAGCA |
| | | | | CCACUGA |
| 321 | HSV-2 | HSV-2 | Version | AUGGCUCUGGGCAGAGUUGGACUGGCUGUUG |
| | gC | gC | 2.3 | GACUGUGGGGACUGCUGUGGGUUGGAGUGGU |
| | | | | GGUGGUGCUGGCUAAUGCUAGCCCCGGCAGA |
| | | | | ACCAUAACAGUAGGGCCACGGGGGAAUGCUU |
| | | | | CCAAUGCUGCACCUUCCGCUUCACCGAGGAA |
| | | | | UGCUUCUGCCCCAAGAACUACCCCCACUCCUC |
| | | | | CUCAACCCAGGAAAGCGACAAAGUCCAAGGC |
| | | | | CAGCACCGCAAAACCCGCUCCUCCUCCAAAGA |
| | | | | CUGGGCCCCCAAAGACAAGUAGCGAACCAGU |
| | | | | UCGGUGCAACAGGCAUGACCCACUUGCACGC |
| | | | | UAUGGGUCAAGAGUCCAGAUACGGUGUCGCU |
| | | | | UCCCUAACAGUACAAGGACUGAGUUUCGGCU |
| | | | | GCAGAUCUGGCGUUAUGCCACAGCUACUGAC |
| | | | | GCAGAGAUUGGUACCGCCCCCAGUUUGGAGG |
| | | | | AAGUGAUGGUCAACGUGUCCGCACCCCCAGG |
| | | | | AGGACAGCUGGUCUAUGACUCAGCGCCCAAU |
| | | | | AGGACCGAUCCCCACGUGAUCUGGGCAGAAG |
| | | | | GAGCCGGUCCUGGGGCCUCUCCACGGCUGUA |
| | | | | CUCAGUUGUUGGCCCGCUUGGACGACAGAGA |
| | | | | CUCAUCAUCGAGGAACUGACACUGGAGACAC |
| | | | | AGGGGAUGUACUACUGGGUGUGGGGCCGUAC |
| | | | | UGACCGCCCUUCCGCAUAUGGCACUUGGGUG |
| | | | | AGAGUUCGCGUCUUUCGGCCCCCUUCUCUCA |
| | | | | CCAUCCAUCCUCAUGCCGUGCUCGAAGGCCA |
| | | | | GCCCUUUAAGGCCACAUGCACUGCUGCGACC |
| | | | | UACUACCCUGGCAACAGAGCCGAGUUUGUCU |
| | | | | GGUUUGAGGAUGGUCGGCGAGUAUUCGAUCC |
| | | | | AGCCCAGAUUCACACACAAACGCAGGAAAAU |
| | | | | CCGGACGGCUUCAGCACAGUGUCCACGGUGA |
| | | | | CCUCUGCUGCAGUUGGUGGACAAGGACCCCC |
| | | | | UCGAACCUUCACCUGUCAGCUGACCUGGCAC |
| | | | | AGAGACUCCGUAAGCUUCAGCCGUAGAAACG |
| | | | | CCUCUGGAACCGCCAGUGUGUUGCCGAGGCC |
| | | | | GACUAUCACGAUGGAAUUCACAGGCGAUCAU |
| | | | | GCCGUCUGUACUGCCGGCUGUGUGCCAGAAG |
| | | | | GCGUAACCUUCGCUUGGUUUCUCGGGGAUGA |
| | | | | CUCAAGUCCUGCAGAGAAAGUGGCUGUGGCC |
| | | | | UCUCAGACGAGCUGCGGUCGACCAGGAACAG |
| | | | | CUACCAUUCGCAGCACUCUGCCCGUGUCCUAC |
| | | | | GAGCAGACGGAGUACAUCUGCAGGCUGGCCG |
| | | | | GCUAUCCCGAUGGGAUUCCAGUCCUGGAGCA |
| | | | | CCACUGA |
| 189 | HSV-1 | HSV-2 | Version | AUGGGCGGAGCUGCUGCUAGACUGGGAGCCG |
| | gD | gC | 1 | UGAUCCUGUUCGUGGUUAUCGUGGGACUGCA |
| | | | | UGGCGUGCGGGGCAAGUAUAGCCCUGGCAGA |
| | | | | ACCAUCACAGUGGGCCCUAGAGGCAACGCCU |
| | | | | CUAAUGCCGCUCCUAGCGCCUCUCCUAGAAA |
| | | | | CGCCUCUGCUCCCAGAACCACACCUACACCUC |
| | | | | CACAGCCUAGAAAGGCCACCAAGAGCAAGGC |
| | | | | CAGCACAGCCAAACCUGCUCCUCCACCUAAGA |
| | | | | CAGGCCCUCCAAAGACAAGCUCUGAGCCCGU |
| | | | | GCGGUGCAACAGACACGAUCCACUGGCCAGA |
| | | | | UACGGCAGCCGGGUGCAGAUCAGAUGCAGAU |
| | | | | UCCCCAACAGCACCCGGACCGAGUUCCGGCUC |
| | | | | CAGAUUUGGAGAUACGCCACCGCCACAGAUG |
| | | | | CCGAGAUUGGAACAGCCCCUAGCCUGGAAGA |
| | | | | AGUGAUGGUCAACGUUUCAGCCCCUCCUGGC |
| | | | | GGCCAGCUGGUGUAUGAUUCUGCCCCUAACC |
| | | | | GGACCGAUCCUCACGUGAUAUGGGCUGAAGG |
| | | | | UGCUGGCCCUGGCGCUUCCCCUAGACUGUAU |
| | | | | UCUGUUGUGGGCCCUCUGGGCAGACAGCGGC |
| | | | | UGAUCAUUGAGGAACUGACCCUGGAAACCCA |
| | | | | GGGCAUGUACUACUGGGUCUGGGGCAGAACC |
| | | | | GAUAGACCAAGCGCCUAUGGCACCUGGGUUC |
| | | | | GAGUGCGAGUGUUCAGACCUCCUAGCCUGAC |
| | | | | CAUCCAUCCUCACGCCGUUCUGGAAGGCCAG |
| | | | | CCUUUCAAGGCCACAUGUACCGCCGCCACCUA |
| | | | | CUAUCCCGGAAACAGAGCCGAGUUCGUUUGG |
| | | | | UUCGAGGACGGCAGAAGGGUGUUCGACCCCG |
| | | | | CUCAGAUCCACACACAGACCCAAGAGAACCCC |
| | | | | GACGGCUUUAGCACCGUGUCCACAGUGACAU |
| | | | | CUGCCGCCGUUGGAGGACAGGGCCCUCCUAG |
| | | | | AACCUUUACCUGCCAGCUGACCUGGCACAGA |
| | | | | GACAGCGUGUCCUUCAGCAGAAGAAACGCCA |
| | | | | GCGGCACAGCCAGCGUUCUGCCUAGACCUAC |
| | | | | CAUCACCAUGGAAUUCACCGGCGACCACGCC |
| | | | | GUGUGUACAGCUGGAUGUGUUCCUGAGGGCG |
| | | | | UGACCUUCGCUUGGUUUCUGGGCGACGAUAG |
| | | | | CAGCCCUGCCGAAAAAGUGGCUGUGGCCAGC |
| | | | | CAGACAAGCUGUGGCAGACCUGGAACCGCCA |
| | | | | CCAUCAGAAGCACACUGCCUGUCAGCUACGA |
| | | | | GCAGACCGAGUACAUCUGUCGGCUGGCCGGC |
| | | | | UAUCCUGAUGGCAUCCCUGUGCUGGAACACC |
| | | | | ACUGAUAA |
| 190 | HSV-1 | HSV-2 | Version | AUGGGAGGAGCAGCUGCCAGACUCGGUGCCG |
| | gD | gC | 2 | UGAUCCUGUUCGUGGUCAUUGUUGGCCUGCA |
| | | | | CGGGGUAAGGGGCAAGUACAGCCCCGGCAGA |
| | | | | ACCAUAACAGUAGGGCCACGGGGGAAUGCUU |
| | | | | CCAAUGCUGCACCUUCCGCUUCACCGAGGAA |
| | | | | UGCUUCUGCCCCAAGAACUACCCCCACUCCUC |
| | | | | CUCAACCCAGGAAAGCGACAAAGUCCAAGGC |
| | | | | CAGCACCGCAAAACCCGCUCCUCCUCCAAAGA |
| | | | | CUGGGCCCCCAAAGACAAGUAGCGAACCAGU |
| | | | | UCGGUGCAACAGGCAUGACCCACUUGCACGC |
| | | | | UAUGGGUCAAGAGUCCAGAUACGGUGUCGCU |
| | | | | UCCCUAACAGUACAAGGACUGAGUUUCGGCU |
| | | | | GCAGAUCUGGCGUUAUGCCACAGCUACUGAC |
| | | | | GCAGAGAUUGGUACCGCCCCCAGUUUGGAAG |
| | | | | AGGUGAUGGUCAACGUGUCCGCACCCCCAGG |
| | | | | AGGACAGCUGGUCUAUGACUCAGCGCCCAAU |
| | | | | AGGACCGAUCCCCACGUGAUCUGGGCAGAAG |
| | | | | GAGCCGGUCCUGGGGCCUCUCCACGGCUGUA |
| | | | | CUCAGUUGUUGGCCCGCUUGGACGACAGAGA |
| | | | | CUCAUCAUCGAAGAGCUGACACUGGAGACAC |
| | | | | AGGGGAUGUACUACUGGGUGUGGGGCCGUAC |
| | | | | UGACCGCCCUUCCGCAUAUGGCACUUGGGUG |
| | | | | AGAGUUCGCGUCUUUCGGCCCCCUUCUCUCA |
| | | | | CCAUCCAUCCUCAUGCCGUGCUCGAAGGCCA |
| | | | | GCCCUUUAAGGCCACAUGCACUGCUGCGACC |
| | | | | UACUACCCUGGCAACAGAGCCGAGUUUGUCU |
| | | | | GGUUUGAGGAUGGUCGGCGAGUAUUCGAUCC |
| | | | | AGCCCAGAUUCACACACAAACGCAGGAAAAU |
| | | | | CCGGACGGCUUCAGCACAGUGUCCACGGUGA |
| | | | | CCUCUGCUGCAGUUGGUGGACAAGGACCCCC |
| | | | | UCGAACCUUCACCUGUCAGCUGACCUGGCAC |
| | | | | AGAGACUCCGUAAGCUUCAGCCGUAGAAACG |
| | | | | CCUCUGGAACCGCCAGUGUGUUGCCGAGGCC |
| | | | | GACUAUCACGAUGGAAUUCACAGGCGAUCAU |
| | | | | GCCGUCUGUACUGCCGGCUGUGUGCCAGAAG |
| | | | | GCGUAACCUUCGCUUGGUUUCUCGGGGAUGA |
| | | | | CUCAAGUCCUGCAGAGAAAGUGGCUGUGGCC |
| | | | | UCUCAGACGAGCUGCGGUCGACCAGGAACAG |
| | | | | CUACCAUUCGCAGCACUCUGCCCGUGUCCUAC |
| | | | | GAGCAGACGGAGUACAUCUGCAGGCUGGCCG |
| | | | | GCUAUCCCGAUGGGAUUCCAGUCCUGGAGCA |
| | | | | CCACUGAUAA |
| 348 | HSV-1 | HSV-2 | Version | AUGGGCGGCGCAGCCGCUAGACUGGGUGCAG |
| | gD | gC | 2.1 | UCAUCCUCUUUGUGGUGAUCGUGGGGCUGCA |
| | | | | UGGGGUCAGAGGGAAGUAUAGCCCCGGCAGA |
| | | | | ACCAUAACAGUAGGGCCACGGGGGAAUGCUU |
| | | | | CCAAUGCUGCACCUUCCGCUUCACCGAGGAA |
| | | | | UGCUUCUGCCCCAAGAACUACCCCCACUCCUC |
| | | | | CUCAACCCAGGAAAGCGACAAAGUCCAAGGC |
| | | | | CAGCACCGCAAAACCCGCUCCUCCUCCAAAGA |
| | | | | CUGGGCCCCCAAAGACAAGUAGCGAACCAGU |
| | | | | UCGGUGCAACAGGCAUGACCCACUUGCACGC |
| | | | | UAUGGGUCAAGAGUCCAGAUACGGUGUCGCU |
| | | | | UCCCUAACAGUACAAGGACUGAGUUUCGGCU |
| | | | | GCAGAUCUGGCGUUAUGCCACAGCUACUGAC |
| | | | | GCAGAGAUUGGUACCGCCCCCAGUUUGGAGG |
| | | | | AAGUGAUGGUCAACGUGUCCGCACCCCCAGG |
| | | | | AGGACAGCUGGUCUAUGACUCAGCGCCCAAU |
| | | | | AGGACCGAUCCCCACGUGAUCUGGGCAGAAG |
| | | | | GAGCCGGUCCUGGGGCCUCUCCACGGCUGUA |
| | | | | CUCAGUUGUUGGCCCGCUUGGACGACAGAGA |
| | | | | CUCAUCAUCGAGGAACUGACACUGGAGACAC |
| | | | | AGGGGAUGUACUACUGGGUGUGGGGCCGUAC |
| | | | | UGACCGCCCUUCCGCAUAUGGCACUUGGGUG |
| | | | | AGAGUUCGCGUCUUUCGGCCCCCUUCUCUCA |
| | | | | CCAUCCAUCCUCAUGCCGUGCUCGAAGGCCA |
| | | | | GCCCUUUAAGGCCACAUGCACUGCUGCGACC |
| | | | | UACUACCCUGGCAACAGAGCCGAGUUUGUCU |
| | | | | GGUUUGAGGAUGGUCGGCGAGUAUUCGAUCC |
| | | | | AGCCCAGAUUCACACACAAACGCAGGAAAAU |
| | | | | CCGGACGGCUUCAGCACAGUGUCCACGGUGA |
| | | | | CCUCUGCUGCAGUUGGUGGACAAGGACCCCC |
| | | | | UCGAACCUUCACCUGUCAGCUGACCUGGCAC |
| | | | | AGAGACUCCGUAAGCUUCAGCCGUAGAAACG |
| | | | | CCUCUGGAACCGCCAGUGUGUUGCCGAGGCC |
| | | | | GACUAUCACGAUGGAAUUCACAGGCGAUCAU |
| | | | | GCCGUCUGUACUGCCGGCUGUGUGCCAGAAG |
| | | | | GCGUAACCUUCGCUUGGUUUCUCGGGGAUGA |
| | | | | CUCAAGUCCUGCAGAGAAAGUGGCUGUGGCC |
| | | | | UCUCAGACGAGCUGCGGUCGACCAGGAACAG |
| | | | | CUACCAUUCGCAGCACUCUGCCCGUGUCCUAC |
| | | | | GAGCAGACGGAGUACAUCUGCAGGCUGGCCG |
| | | | | GCUAUCCCGAUGGGAUUCCAGUCCUGGAGCA |
| | | | | CCACUGAUAA |
| 349 | HSV-1 | HSV-2 | Version | AUGGGAGGAGCAGCUGCCAGACUCGGUGCCG |
| | gD | gC | 2.2 | UGAUCCUGUUCGUGGUCAUUGUUGGCCUGCA |
| | | | | CGGGGUAAGGGGCAAGUACAGCCCCGGCAGA |
| | | | | ACCAUAACAGUAGGGCCACGGGGGAAUGCUU |
| | | | | CCAAUGCUGCACCUUCCGCUUCACCGAGGAA |
| | | | | UGCUUCUGCCCCAAGAACUACCCCCACUCCUC |
| | | | | CUCAACCCAGGAAAGCGACAAAGUCCAAGGC |
| | | | | CAGCACCGCAAAACCCGCUCCUCCUCCAAAGA |
| | | | | CUGGGCCCCCAAAGACAAGUAGCGAACCAGU |
| | | | | UCGGUGCAACAGGCAUGACCCACUUGCACGC |
| | | | | UAUGGGUCAAGAGUCCAGAUACGGUGUCGCU |
| | | | | UCCCUAACAGUACAAGGACUGAGUUUCGGCU |
| | | | | GCAGAUCUGGCGUUAUGCCACAGCUACUGAC |
| | | | | GCAGAGAUUGGUACCGCCCCCAGUUUGGAGG |
| | | | | AAGUGAUGGUCAACGUGUCCGCACCCCCAGG |
| | | | | AGGACAGCUGGUCUAUGACUCAGCGCCCAAU |
| | | | | AGGACCGAUCCCCACGUGAUCUGGGCAGAAG |
| | | | | GAGCCGGUCCUGGGGCCUCUCCACGGCUGUA |
| | | | | CUCAGUUGUUGGCCCGCUUGGACGACAGAGA |
| | | | | CUCAUCAUCGAGGAACUGACACUGGAGACAC |
| | | | | AGGGGAUGUACUACUGGGUGUGGGGCCGUAC |
| | | | | UGACCGCCCUUCCGCAUAUGGCACUUGGGUG |
| | | | | AGAGUUCGCGUCUUUCGGCCCCCUUCUCUCA |
| | | | | CCAUCCAUCCUCAUGCCGUGCUCGAAGGCCA |
| | | | | GCCCUUUAAGGCCACAUGCACUGCUGCGACC |
| | | | | UACUACCCUGGCAACAGAGCCGAGUUUGUCU |
| | | | | GGUUUGAGGAUGGUCGGCGAGUAUUCGAUCC |
| | | | | AGCCCAGAUUCACACACAAACGCAGGAAAAU |
| | | | | CCGGACGGCUUCAGCACAGUGUCCACGGUGA |
| | | | | CCUCUGCUGCAGUUGGUGGACAAGGACCCCC |
| | | | | UCGAACCUUCACCUGUCAGCUGACCUGGCAC |
| | | | | AGAGACUCCGUAAGCUUCAGCCGUAGAAACG |
| | | | | CCUCUGGAACCGCCAGUGUGUUGCCGAGGCC |
| | | | | GACUAUCACGAUGGAAUUCACAGGCGAUCAU |
| | | | | GCCGUCUGUACUGCCGGCUGUGUGCCAGAAG |
| | | | | GCGUAACCUUCGCUUGGUUUCUCGGGGAUGA |
| | | | | CUCAAGUCCUGCAGAGAAAGUGGCUGUGGCC |
| | | | | UCUCAGACGAGCUGCGGUCGACCAGGAACAG |
| | | | | CUACCAUUCGCAGCACUCUGCCCGUGUCCUAC |
| | | | | GAGCAGACGGAGUACAUCUGCAGGCUGGCCG |
| | | | | GCUAUCCCGAUGGGAUUCCAGUCCUGGAGCA |
| | | | | CCACUGAUAA |
| 191 | HSV-1 | HSV-2 | Version | AUGGGAGGAGCUGCUGCUAGAUUGGGAGCUG |
| | gD | gC | 3 | UGAUUCUGUUUGUGGUGAUUGUGGGACUGCA |
| | | | | UGGAGUGAGAGGAAAAUACUCUCCUGGAAGA |
| | | | | ACCAUCACAGUGGGACCAAGAGGAAAUGCAA |
| | | | | GCAAUGCAGCACCUUCUGCUUCUCCAAGAAA |
| | | | | UGCUUCUGCUCCAAGAACCACCCCAACCCCUC |
| | | | | CUCAGCCAAGAAAAGCAACCAAAUCCAAAGC |
| | | | | AUCCACAGCAAAACCUGCACCUCCUCCAAAA |
| | | | | ACAGGACCUCCAAAAACCUCCUCUGAACCUG |
| | | | | UGAGAUGCAACAGACAUGAUCCUCUGGCAAG |
| | | | | AUAUGGAUCAAGAGUGCAGAUCAGAUGCAGA |
| | | | | UUUCCAAAUUCCACCAGAACAGAAUUCAGAC |
| | | | | UCCAGAUCUGGAGAUAUGCAACAGCAACAGA |
| | | | | UGCAGAAAUUGGAACAGCACCAUCUCUGGAA |
| | | | | GAAGUGAUGGUGAAUGUGUCUGCUCCUCCUG |
| | | | | GAGGACAGCUGGUGUAUGAUUCUGCUCCAAA |
| | | | | CAGAACAGAUCCUCAUGUGAUCUGGGCUGAA |
| | | | | GGAGCUGGACCUGGAGCUUCUCCAAGACUGU |
| | | | | ACUCUGUGGUGGGACCUCUGGGAAGACAGAG |
| | | | | ACUGAUCAUUGAAGAACUGACCCUGGAAACC |
| | | | | CAGGGAAUGUACUACUGGGUGUGGGGAAGAA |
| | | | | CAGACAGACCUUCUGCUUAUGGAACCUGGGU |
| | | | | GAGAGUGAGAGUGUUCAGACCUCCUUCUCUG |
| | | | | ACCAUCCACCCUCAUGCUGUGCUGGAAGGAC |
| | | | | AGCCUUUCAAAGCAACCUGCACAGCAGCAAC |
| | | | | CUACUACCCUGGAAACAGAGCUGAAUUUGUG |
| | | | | UGGUUUGAAGAUGGAAGAAGGGUGUUUGAUC |
| | | | | CUGCUCAGAUCCACACCCAGACCCAGGAAAA |
| | | | | UCCUGAUGGAUUUUCCACAGUGUCCACAGUG |
| | | | | ACAUCUGCUGCUGUGGGAGGACAGGGACCUC |
| | | | | CAAGAACCUUCACCUGCCAGCUGACCUGGCA |
| | | | | CAGAGAUUCUGUGUCUUUUUCAAGAAGAAAU |
| | | | | GCUUCUGGAACAGCUUCUGUGCUGCCAAGAC |
| | | | | CAACCAUCACCAUGGAAUUCACAGGAGAUCA |
| | | | | UGCUGUGUGCACAGCUGGAUGUGUGCCUGAA |
| | | | | GGAGUGACCUUUGCUUGGUUUCUGGGAGAUG |
| | | | | AUUCUUCUCCAGCUGAAAAAGUGGCUGUGGC |
| | | | | UUCCCAGACCUCUUGUGGAAGACCUGGAACA |
| | | | | GCAACCAUCAGAUCCACCCUGCCUGUGUCUU |
| | | | | AUGAACAGACAGAAUACAUUUGCAGACUGGC |
| | | | | UGGAUACCCUGAUGGAAUCCCUGUGCUGGAA |
| | | | | CACCACUGAUAA |
| 192 | HSV-1 | HSV-2 | Version | AUGGGCGGCGCAGCCGCUAGACUGGGUGCAG |
| | gD | gC | 4 | UCAUCCUCUUUGUGGUGAUCGUGGGGCUGCA |
| | | | | UGGGGUCAGAGGGAAGUAUUCUCCGGGACGG |
| | | | | ACUAUAACCGUAGGUCCAAGAGGAAACGCCU |
| | | | | CUAACGCAGCCCCGUCUGCCUCACCACGAAAC |
| | | | | GCCUCAGCUCCCAGAACCACUCCUACUCCACC |
| | | | | CCAGCCUAGGAAGGCGACGAAAUCCAAGGCU |
| | | | | UCCACGGCCAAACCCGCCCCUCCACCCAAAAC |
| | | | | CGGACCUCCUAAGACCAGCUCUGAACCGGUG |
| | | | | CGGUGUAAUAGGCACGACCCAUUGGCGCGAU |
| | | | | AUGGCAGUAGGGUCCAGAUACGGUGCAGAUU |
| | | | | CCCAAACAGCACAAGAACAGAAUUCCGGCUG |
| | | | | CAAAUCUGGCGAUAUGCAACGGCCACCGAUG |
| | | | | CCGAAAUCGGAACAGCACCCAGUCUGGAAGA |
| | | | | AGUGAUGGUGAACGUCAGUGCUCCACCUGGC |
| | | | | GGACAACUGGUGUACGACUCUGCACCCAAUC |
| | | | | GCACAGAUCCCCACGUGAUUUGGGCCGAGGG |
| | | | | UGCUGGACCUGGGGCUUCACCCAGGCUGUAU |
| | | | | AGCGUUGUAGGGCCACUUGGGAGGCAGAGAC |
| | | | | UCAUCAUUGAGGAACUGACCCUGGAAACUCA |
| | | | | GGGCAUGUACUACUGGGUAUGGGGCCGCACA |
| | | | | GAUCGCCCCAGCGCUUAUGGCACCUGGGUGC |
| | | | | GGGUGCGGGUGUUUCGCCCACCCUCCCUCACC |
| | | | | AUUCACCCUCAUGCGGUUCUGGAGGGACAGC |
| | | | | CUUUCAAGGCAACUUGUACCGCAGCCACCUA |
| | | | | CUAUCCCGGCAAUAGAGCGGAGUUCGUCUGG |
| | | | | UUUGAGGACGGCCGUAGGGUGUUCGAUCCUG |
| | | | | CCCAGAUUCACACCCAGACACAGGAGAAUCC |
| | | | | CGACGGCUUUAGCACAGUGAGCACUGUGACG |
| | | | | UCUGCUGCCGUUGGUGGUCAAGGGCCUCCUC |
| | | | | GUACCUUCACAUGCCAAUUGACCUGGCACCG |
| | | | | CGACUCAGUUAGCUUUAGCCGCCGGAAUGCC |
| | | | | AGUGGGACCGCCAGUGUUCUCCCAAGGCCGA |
| | | | | CAAUCACCAUGGAGUUCACUGGCGACCAUGC |
| | | | | AGUGUGCACAGCUGGGUGUGUCCCAGAAGGC |
| | | | | GUGACUUUCGCCUGGUUUCUGGGUGAUGACU |
| | | | | CCUCACCCGCCGAGAAAGUAGCUGUCGCUUC |
| | | | | CCAGACUUCCUGUGGACGUCCUGGAACUGCG |
| | | | | ACAAUCCGAAGCACACUGCCGGUUUCCUACG |
| | | | | AGCAGACGGAGUACAUAUGCCGCCUUGCAGG |
| | | | | CUACCCCGAUGGAAUUCCAGUCCUUGAGCAC |
| | | | | CAUUGA |
| 193 | HSV-1 | HSV-2 | Version | AUGCAUCAGGGCGCUCCAUCUUGGGGUAGAC |
| | gB | gC | 1 | GUUGGUUCGUUGUGUGGGCCCUGCUGGGACU |
| | | | | GACACUGGGAGUUCUGGUUGCCUCUGCUGCU |
| | | | | CCUAGCCCUGGCAGAACCAUCACAGUGGGCC |
| | | | | CUAGAGGCAACGCCUCUAAUGCCGCUCCUAG |
| | | | | CGCCUCUCCUAGAAACGCCUCUGCUCCCAGAA |
| | | | | CCACACCUACACCUCCACAGCCUAGAAAGGCC |
| | | | | ACCAAGAGCAAGGCCAGCACAGCCAAACCUG |
| | | | | CUCCUCCACCUAAGACAGGCCCUCCAAAGACA |
| | | | | AGCUCUGAGCCCGUGCGGUGCAACAGACACG |
| | | | | AUCCACUGGCCAGAUACGGCAGCCGGGUGCA |
| | | | | GAUCAGAUGCAGAUUCCCCAACAGCACCCGG |
| | | | | ACCGAGUUCCGGCUCCAGAUUUGGAGAUACG |
| | | | | CCACCGCCACAGAUGCCGAGAUUGGAACAGC |
| | | | | CCCUAGCCUGGAAGAAGUGAUGGUCAACGUU |
| | | | | UCAGCCCCUCCUGGCGGCCAGCUGGUGUAUG |
| | | | | AUUCUGCCCCUAACCGGACCGAUCCUCACGU |
| | | | | GAUAUGGGCUGAAGGUGCUGGCCCUGGCGCU |
| | | | | UCCCCUAGACUGUAUUCUGUUGUGGGCCCUC |
| | | | | UGGGCAGACAGCGGCUGAUCAUUGAGGAACU |
| | | | | GACCCUGGAAACCCAGGGCAUGUACUACUGG |
| | | | | GUCUGGGGCAGAACCGAUAGACCAAGCGCCU |
| | | | | AUGGCACCUGGGUUCGAGUGCGAGUGUUCAG |
| | | | | ACCUCCUAGCCUGACCAUCCAUCCUCACGCCG |
| | | | | UUCUGGAAGGCCAGCCUUUCAAGGCCACAUG |
| | | | | UACCGCCGCCACCUACUAUCCCGGAAACAGA |
| | | | | GCCGAGUUCGUUUGGUUCGAGGACGGCAGAA |
| | | | | GGGUGUUCGACCCCGCUCAGAUCCACACACA |
| | | | | GACCCAAGAGAACCCCGACGGCUUUAGCACC |
| | | | | GUGUCCACAGUGACAUCUGCCGCCGUUGGAG |
| | | | | GACAGGGCCCUCCUAGAACCUUUACCUGCCA |
| | | | | GCUGACCUGGCACAGAGACAGCGUGUCCUUC |
| | | | | AGCAGAAGAAACGCCAGCGGCACAGCCAGCG |
| | | | | UUCUGCCUAGACCUACCAUCACCAUGGAAUU |
| | | | | CACCGGCGACCACGCCGUGUGUACAGCUGGA |
| | | | | UGUGUUCCUGAGGGCGUGACCUUCGCUUGGU |
| | | | | UUCUGGGCGACGAUAGCAGCCCUGCCGAAAA |
| | | | | AGUGGCUGUGGCCAGCCAGACAAGCUGUGGC |
| | | | | AGACCUGGAACCGCCACCAUCAGAAGCACAC |
| | | | | UGCCUGUCAGCUACGAGCAGACCGAGUACAU |
| | | | | CUGUCGGCUGGCCGGCUAUCCUGAUGGCAUC |
| | | | | CCUGUGCUGGAACACCACUGAUAA |
| 194 | HSV-1 | HSV-2 | Version | AUGCACCAGGGUGCCCCUUCCUGGGGCAGAA |
| | gB | gC | 2 | GGUGGUUCGUGGUGUGGGCCCUUCUGGGGCU |
| | | | | GACCCUCGGAGUCCUGGUUGCGAGCGCAGCU |
| | | | | CCCAGCCCCGGCAGAACCAUAACAGUAGGGC |
| | | | | CACGGGGGAAUGCUUCCAAUGCUGCACCUUC |
| | | | | CGCUUCACCGAGGAAUGCUUCUGCCCCAAGA |
| | | | | ACUACCCCCACUCCUCCUCAACCCAGGAAAGC |
| | | | | GACAAAGUCCAAGGCCAGCACCGCAAAACCC |
| | | | | GCUCCUCCUCCAAAGACUGGGCCCCCAAAGAC |
| | | | | AAGUAGCGAACCAGUUCGGUGCAACAGGCAU |
| | | | | GACCCACUUGCACGCUAUGGGUCAAGAGUCC |
| | | | | AGAUACGGUGUCGCUUCCCUAACAGUACAAG |
| | | | | GACUGAGUUUCGGCUGCAGAUCUGGCGUUAU |
| | | | | GCCACAGCUACUGACGCAGAGAUUGGUACCG |
| | | | | CCCCCAGUUUGGAAGAGGUGAUGGUCAACGU |
| | | | | GUCCGCACCCCCAGGAGGACAGCUGGUCUAU |
| | | | | GACUCAGCGCCCAAUAGGACCGAUCCCCACG |
| | | | | UGAUCUGGGCAGAAGGAGCCGGUCCUGGGGC |
| | | | | CUCUCCACGGCUGUACUCAGUUGUUGGCCCG |
| | | | | CUUGGACGACAGAGACUCAUCAUCGAAGAGC |
| | | | | UGACACUGGAGACACAGGGGAUGUACUACUG |
| | | | | GGUGUGGGGCCGUACUGACCGCCCUUCCGCA |
| | | | | UAUGGCACUUGGGUGAGAGUUCGCGUCUUUC |
| | | | | GGCCCCCUUCUCUCACCAUCCAUCCUCAUGCC |
| | | | | GUGCUCGAAGGCCAGCCCUUUAAGGCCACAU |
| | | | | GCACUGCUGCGACCUACUACCCUGGCAACAG |
| | | | | AGCCGAGUUUGUCUGGUUUGAGGAUGGUCGG |
| | | | | CGAGUAUUCGAUCCAGCCCAGAUUCACACAC |
| | | | | AAACGCAGGAAAAUCCGGACGGCUUCAGCAC |
| | | | | AGUGUCCACGGUGACCUCUGCUGCAGUUGGU |
| | | | | GGACAAGGACCCCCUCGAACCUUCACCUGUC |
| | | | | AGCUGACCUGGCACAGAGACUCCGUAAGCUU |
| | | | | CAGCCGUAGAAACGCCUCUGGAACCGCCAGU |
| | | | | GUGUUGCCGAGGCCGACUAUCACGAUGGAAU |
| | | | | UCACAGGCGAUCAUGCCGUCUGUACUGCCGG |
| | | | | CUGUGUGCCAGAAGGCGUAACCUUCGCUUGG |
| | | | | UUUCUCGGGGAUGACUCAAGUCCUGCAGAGA |
| | | | | AAGUGGCUGUGGCCUCUCAGACGAGCUGCGG |
| | | | | UCGACCAGGAACAGCUACCAUUCGCAGCACU |
| | | | | CUGCCCGUGUCCUACGAGCAGACGGAGUACA |
| | | | | UCUGCAGGCUGGCCGGCUAUCCCGAUGGGAU |
| | | | | UCCAGUCCUGGAGCACCACUGAUAA |
| 350 | HSV-1 | HSV-2 | Version | AUGCAUCAAGGCGCACCCAGUUGGGGCAGAC |
| | ?B | gC | 2.1 | GGUGGUUCGUAGUGUGGGCCUUGCUGGGACU |
| | | | | GACUCUCGGUGUCCUCGUUGCGUCUGCUGCA |
| | | | | CCGAGCCCCGGCAGAACCAUAACAGUAGGGC |
| | | | | CACGGGGGAAUGCUUCCAAUGCUGCACCUUC |
| | | | | CGCUUCACCGAGGAAUGCUUCUGCCCCAAGA |
| | | | | ACUACCCCCACUCCUCCUCAACCCAGGAAAGC |
| | | | | GACAAAGUCCAAGGCCAGCACCGCAAAACCC |
| | | | | GCUCCUCCUCCAAAGACUGGGCCCCCAAAGAC |
| | | | | AAGUAGCGAACCAGUUCGGUGCAACAGGCAU |
| | | | | GACCCACUUGCACGCUAUGGGUCAAGAGUCC |
| | | | | AGAUACGGUGUCGCUUCCCUAACAGUACAAG |
| | | | | GACUGAGUUUCGGCUGCAGAUCUGGCGUUAU |
| | | | | GCCACAGCUACUGACGCAGAGAUUGGUACCG |
| | | | | CCCCCAGUUUGGAGGAAGUGAUGGUCAACGU |
| | | | | GUCCGCACCCCCAGGAGGACAGCUGGUCUAU |
| | | | | GACUCAGCGCCCAAUAGGACCGAUCCCCACG |
| | | | | UGAUCUGGGCAGAAGGAGCCGGUCCUGGGGC |
| | | | | CUCUCCACGGCUGUACUCAGUUGUUGGCCCG |
| | | | | CUUGGACGACAGAGACUCAUCAUCGAGGAAC |
| | | | | UGACACUGGAGACACAGGGGAUGUACUACUG |
| | | | | GGUGUGGGGCCGUACUGACCGCCCUUCCGCA |
| | | | | UAUGGCACUUGGGUGAGAGUUCGCGUCUUUC |
| | | | | GGCCCCCUUCUCUCACCAUCCAUCCUCAUGCC |
| | | | | GUGCUCGAAGGCCAGCCCUUUAAGGCCACAU |
| | | | | GCACUGCUGCGACCUACUACCCUGGCAACAG |
| | | | | AGCCGAGUUUGUCUGGUUUGAGGAUGGUCGG |
| | | | | CGAGUAUUCGAUCCAGCCCAGAUUCACACAC |
| | | | | AAACGCAGGAAAAUCCGGACGGCUUCAGCAC |
| | | | | AGUGUCCACGGUGACCUCUGCUGCAGUUGGU |
| | | | | GGACAAGGACCCCCUCGAACCUUCACCUGUC |
| | | | | AGCUGACCUGGCACAGAGACUCCGUAAGCUU |
| | | | | CAGCCGUAGAAACGCCUCUGGAACCGCCAGU |
| | | | | GUGUUGCCGAGGCCGACUAUCACGAUGGAAU |
| | | | | UCACAGGCGAUCAUGCCGUCUGUACUGCCGG |
| | | | | CUGUGUGCCAGAAGGCGUAACCUUCGCUUGG |
| | | | | UUUCUCGGGGAUGACUCAAGUCCUGCAGAGA |
| | | | | AAGUGGCUGUGGCCUCUCAGACGAGCUGCGG |
| | | | | UCGACCAGGAACAGCUACCAUUCGCAGCACU |
| | | | | CUGCCCGUGUCCUACGAGCAGACGGAGUACA |
| | | | | UCUGCAGGCUGGCCGGCUAUCCCGAUGGGAU |
| | | | | UCCAGUCCUGGAGCACCACUGAUAA |
| 195 | HSV-1 | HSV-2 | Version | AUGCACCAGGGAGCACCUUCUUGGGGAAGAA |
| | gB | gC | 3 | GAUGGUUUGUGGUGUGGGCUCUGCUGGGACU |
| | | | | GACCCUGGGAGUGCUGGUGGCUUCUGCUGCU |
| | | | | CCUUCUCCUGGAAGAACCAUCACAGUGGGAC |
| | | | | CAAGAGGAAAUGCAAGCAAUGCAGCACCUUC |
| | | | | UGCUUCUCCAAGAAAUGCUUCUGCUCCAAGA |
| | | | | ACCACCCCAACCCCUCCUCAGCCAAGAAAAGC |
| | | | | AACCAAAUCCAAAGCAUCCACAGCAAAACCU |
| | | | | GCACCUCCUCCAAAAACAGGACCUCCAAAAA |
| | | | | CCUCCUCUGAACCUGUGAGAUGCAACAGACA |
| | | | | UGAUCCUCUGGCAAGAUAUGGAUCAAGAGUG |
| | | | | CAGAUCAGAUGCAGAUUUCCAAAUUCCACCA |
| | | | | GAACAGAAUUCAGACUCCAGAUCUGGAGAUA |
| | | | | UGCAACAGCAACAGAUGCAGAAAUUGGAACA |
| | | | | GCACCAUCUCUGGAAGAAGUGAUGGUGAAUG |
| | | | | UGUCUGCUCCUCCUGGAGGACAGCUGGUGUA |
| | | | | UGAUUCUGCUCCAAACAGAACAGAUCCUCAU |
| | | | | GUGAUCUGGGCUGAAGGAGCUGGACCUGGAG |
| | | | | CUUCUCCAAGACUGUACUCUGUGGUGGGACC |
| | | | | UCUGGGAAGACAGAGACUGAUCAUUGAAGAA |
| | | | | CUGACCCUGGAAACCCAGGGAAUGUACUACU |
| | | | | GGGUGUGGGGAAGAACAGACAGACCUUCUGC |
| | | | | UUAUGGAACCUGGGUGAGAGUGAGAGUGUUC |
| | | | | AGACCUCCUUCUCUGACCAUCCACCCUCAUGC |
| | | | | UGUGCUGGAAGGACAGCCUUUCAAAGCAACC |
| | | | | UGCACAGCAGCAACCUACUACCCUGGAAACA |
| | | | | GAGCUGAAUUUGUGUGGUUUGAAGAUGGAAG |
| | | | | AAGGGUGUUUGAUCCUGCUCAGAUCCACACC |
| | | | | CAGACCCAGGAAAAUCCUGAUGGAUUUUCCA |
| | | | | CAGUGUCCACAGUGACAUCUGCUGCUGUGGG |
| | | | | AGGACAGGGACCUCCAAGAACCUUCACCUGC |
| | | | | CAGCUGACCUGGCACAGAGAUUCUGUGUCUU |
| | | | | UUUCAAGAAGAAAUGCUUCUGGAACAGCUUC |
| | | | | UGUGCUGCCAAGACCAACCAUCACCAUGGAA |
| | | | | UUCACAGGAGAUCAUGCUGUGUGCACAGCUG |
| | | | | GAUGUGUGCCUGAAGGAGUGACCUUUGCUUG |
| | | | | GUUUCUGGGAGAUGAUUCUUCUCCAGCUGAA |
| | | | | AAAGUGGCUGUGGCUUCCCAGACCUCUUGUG |
| | | | | GAAGACCUGGAACAGCAACCAUCAGAUCCAC |
| | | | | CCUGCCUGUGUCUUAUGAACAGACAGAAUAC |
| | | | | AUUUGCAGACUGGCUGGAUACCCUGAUGGAA |
| | | | | UCCCUGUGCUGGAACACCACUGAUAA |
| 196 | HSV-1 | HSV-2 | Version | AUGCAUCAAGGCGCACCCAGUUGGGGCAGAC |
| | gB | gC | 4 | GGUGGUUCGUAGUGUGGGCCUUGCUGGGACU |
| | | | | GACUCUCGGUGUCCUCGUUGCGUCUGCUGCA |
| | | | | CCGAGUCCAGGAAGGACGAUUACGGUGGGAC |
| | | | | CCAGAGGUAAUGCGUCCAAUGCUGCGCCAUC |
| | | | | CGCUUCUCCACGGAACGCAUCUGCACCCAGG |
| | | | | ACUACACCGACACCACCUCAGCCGCGCAAAGC |
| | | | | CACCAAGAGCAAGGCCAGCACAGCCAAACCC |
| | | | | GCUCCUCCACCUAAAACCGGACCACCUAAGAC |
| | | | | CAGCUCUGAACCCGUCAGAUGCAACAGGCAC |
| | | | | GAUCCGUUGGCCAGAUAUGGCAGUCGCGUCC |
| | | | | AGAUCAGGUGUCGCUUCCCUAACAGCACACG |
| | | | | GACCGAGUUCAGGCUGCAAAUUUGGCGCUAC |
| | | | | GCUACAGCCACUGACGCAGAGAUUGGCACUG |
| | | | | CUCCCAGUCUGGAGGAGGUCAUGGUGAACGU |
| | | | | GUCUGCUCCACCAGGCGGUCAGCUGGUCUAU |
| | | | | GACUCAGCCCCUAAUCGCACAGAUCCUCACG |
| | | | | UGAUUUGGGCAGAAGGUGCGGGGCCUGGGGC |
| | | | | CUCCCCAAGGCUCUACUCAGUGGUUGGACCC |
| | | | | CUUGGGAGACAGCGGCUGAUCAUCGAGGAAC |
| | | | | UGACUCUCGAAACCCAAGGUAUGUACUACUG |
| | | | | GGUAUGGGGCAGAACAGACAGACCUUCAGCU |
| | | | | UAUGGCACCUGGGUGCGGGUGAGAGUGUUUA |
| | | | | GGCCUCCCUCCCUGACGAUCCAUCCCCAUGCU |
| | | | | GUGCUGGAAGGACAGCCGUUCAAGGCAACAU |
| | | | | GCACAGCAGCCACUUACUAUCCCGGAAACCG |
| | | | | UGCUGAGUUUGUGUGGUUCGAGGAUGGGCGA |
| | | | | CGUGUAUUCGACCCUGCCCAGAUUCACACCC |
| | | | | AGACACAGGAGAAUCCCGACGGGUUUUCCAC |
| | | | | UGUGAGCACCGUGACAUCAGCGGCAGUAGGA |
| | | | | GGGCAGGGCCCACCCCGAACGUUCACUUGCC |
| | | | | AGCUUACUUGGCAUCGGGACAGUGUUAGCUU |
| | | | | UAGCCGCCGGAAUGCCUCUGGCACCGCAUCC |
| | | | | GUCCUUCCUCGCCCAACCAUCACCAUGGAAU |
| | | | | UCACUGGCGAUCACGCCGUUUGUACAGCCGG |
| | | | | GUGUGUUCCCGAGGGAGUGACCUUUGCUUGG |
| | | | | UUUCUGGGCGAUGACUCAAGCCCAGCCGAAA |
| | | | | AGGUGGCCGUCGCCUCCCAAACGAGCUGUGG |
| | | | | GCGACCUGGCACCGCUACCAUACGUAGCACU |
| | | | | CUGCCCGUUUCCUACGAACAGACCGAGUAUA |
| | | | | UCUGCCGAUUGGCCGGUUACCCCGAUGGGAU |
| | | | | ACCAGUCCUGGAGCACCACUGA |
| 197 | HSV-2 gI | HSV-2 | Version | AUGCCUGGCAGAUCUCUGCAAGGACUGGCCA |
| | | gC | 1 | UCCUCGGACUGUGGGUUUGCGCAACAGGACU |
| | | | | GAGCCCUGGCAGAACCAUCACAGUGGGCCCU |
| | | | | AGAGGCAACGCCUCUAAUGCCGCUCCUAGCG |
| | | | | CCUCUCCUAGAAACGCCUCUGCUCCCAGAACC |
| | | | | ACACCUACACCUCCACAGCCUAGAAAGGCCAC |
| | | | | CAAGAGCAAGGCCAGCACAGCCAAACCUGCU |
| | | | | CCUCCACCUAAGACAGGCCCUCCAAAGACAA |
| | | | | GCUCUGAGCCCGUGCGGUGCAACAGACACGA |
| | | | | UCCACUGGCCAGAUACGGCAGCCGGGUGCAG |
| | | | | AUCAGAUGCAGAUUCCCCAACAGCACCCGGA |
| | | | | CCGAGUUCCGGCUCCAGAUUUGGAGAUACGC |
| | | | | CACCGCCACAGAUGCCGAGAUUGGAACAGCC |
| | | | | CCUAGCCUGGAAGAAGUGAUGGUCAACGUUU |
| | | | | CAGCCCCUCCUGGCGGCCAGCUGGUGUAUGA |
| | | | | UUCUGCCCCUAACCGGACCGAUCCUCACGUG |
| | | | | AUAUGGGCUGAAGGUGCUGGCCCUGGCGCUU |
| | | | | CCCCUAGACUGUAUUCUGUUGUGGGCCCUCU |
| | | | | GGGCAGACAGCGGCUGAUCAUUGAGGAACUG |
| | | | | ACCCUGGAAACCCAGGGCAUGUACUACUGGG |
| | | | | UCUGGGGCAGAACCGAUAGACCAAGCGCCUA |
| | | | | UGGCACCUGGGUUCGAGUGCGAGUGUUCAGA |
| | | | | CCUCCUAGCCUGACCAUCCAUCCUCACGCCGU |
| | | | | UCUGGAAGGCCAGCCUUUCAAGGCCACAUGU |
| | | | | ACCGCCGCCACCUACUAUCCCGGAAACAGAGC |
| | | | | CGAGUUCGUUUGGUUCGAGGACGGCAGAAGG |
| | | | | GUGUUCGACCCCGCUCAGAUCCACACACAGA |
| | | | | CCCAAGAGAACCCCGACGGCUUUAGCACCGU |
| | | | | GUCCACAGUGACAUCUGCCGCCGUUGGAGGA |
| | | | | CAGGGCCCUCCUAGAACCUUUACCUGCCAGC |
| | | | | UGACCUGGCACAGAGACAGCGUGUCCUUCAG |
| | | | | CAGAAGAAACGCCAGCGGCACAGCCAGCGUU |
| | | | | CUGCCUAGACCUACCAUCACCAUGGAAUUCA |
| | | | | CCGGCGACCACGCCGUGUGUACAGCUGGAUG |
| | | | | UGUUCCUGAGGGCGUGACCUUCGCUUGGUUU |
| | | | | CUGGGCGACGAUAGCAGCCCUGCCGAAAAAG |
| | | | | UGGCUGUGGCCAGCCAGACAAGCUGUGGCAG |
| | | | | ACCUGGAACCGCCACCAUCAGAAGCACACUG |
| | | | | CCUGUCAGCUACGAGCAGACCGAGUACAUCU |
| | | | | GUCGGCUGGCCGGCUAUCCUGAUGGCAUCCC |
| | | | | UGUGCUGGAACACCACUGAUAA |
| 198 | HSV-2 gI | HSV-2 | Version | AUGCCCGGCAGAAGCCUCCAGGGACUGGCUA |
| | | gC | 2 | UCCUGGGGCUGUGGGUGUGCGCCACCGGUCU |
| | | | | UAGCCCCGGCAGAACCAUAACAGUAGGGCCA |
| | | | | CGGGGGAAUGCUUCCAAUGCUGCACCUUCCG |
| | | | | CUUCACCGAGGAAUGCUUCUGCCCCAAGAAC |
| | | | | UACCCCCACUCCUCCUCAACCCAGGAAAGCGA |
| | | | | CAAAGUCCAAGGCCAGCACCGCAAAACCCGC |
| | | | | UCCUCCUCCAAAGACUGGGCCCCCAAAGACA |
| | | | | AGUAGCGAACCAGUUCGGUGCAACAGGCAUG |
| | | | | ACCCACUUGCACGCUAUGGGUCAAGAGUCCA |
| | | | | GAUACGGUGUCGCUUCCCUAACAGUACAAGG |
| | | | | ACUGAGUUUCGGCUGCAGAUCUGGCGUUAUG |
| | | | | CCACAGCUACUGACGCAGAGAUUGGUACCGC |
| | | | | CCCCAGUUUGGAAGAGGUGAUGGUCAACGUG |
| | | | | UCCGCACCCCCAGGAGGACAGCUGGUCUAUG |
| | | | | ACUCAGCGCCCAAUAGGACCGAUCCCCACGU |
| | | | | GAUCUGGGCAGAAGGAGCCGGUCCUGGGGCC |
| | | | | UCUCCACGGCUGUACUCAGUUGUUGGCCCGC |
| | | | | UUGGACGACAGAGACUCAUCAUCGAAGAGCU |
| | | | | GACACUGGAGACACAGGGGAUGUACUACUGG |
| | | | | GUGUGGGGCCGUACUGACCGCCCUUCCGCAU |
| | | | | AUGGCACUUGGGUGAGAGUUCGCGUCUUUCG |
| | | | | GCCCCCUUCUCUCACCAUCCAUCCUCAUGCCG |
| | | | | UGCUCGAAGGCCAGCCCUUUAAGGCCACAUG |
| | | | | CACUGCUGCGACCUACUACCCUGGCAACAGA |
| | | | | GCCGAGUUUGUCUGGUUUGAGGAUGGUCGGC |
| | | | | GAGUAUUCGAUCCAGCCCAGAUUCACACACA |
| | | | | AACGCAGGAAAAUCCGGACGGCUUCAGCACA |
| | | | | GUGUCCACGGUGACCUCUGCUGCAGUUGGUG |
| | | | | GACAAGGACCCCCUCGAACCUUCACCUGUCA |
| | | | | GCUGACCUGGCACAGAGACUCCGUAAGCUUC |
| | | | | AGCCGUAGAAACGCCUCUGGAACCGCCAGUG |
| | | | | UGUUGCCGAGGCCGACUAUCACGAUGGAAUU |
| | | | | CACAGGCGAUCAUGCCGUCUGUACUGCCGGC |
| | | | | UGUGUGCCAGAAGGCGUAACCUUCGCUUGGU |
| | | | | UUCUCGGGGAUGACUCAAGUCCUGCAGAGAA |
| | | | | AGUGGCUGUGGCCUCUCAGACGAGCUGCGGU |
| | | | | CGACCAGGAACAGCUACCAUUCGCAGCACUC |
| | | | | UGCCCGUGUCCUACGAGCAGACGGAGUACAU |
| | | | | CUGCAGGCUGGCCGGCUAUCCCGAUGGGAUU |
| | | | | CCAGUCCUGGAGCACCACUGAUAA |
| 199 | HSV-2 gI | HSV-2 | Version | AUGCCUGGAAGAUCUCUCCAGGGACUGGCAA |
| | | gC | 3 | UCCUGGGACUGUGGGUGUGUGCAACAGGACU |
| | | | | GUCUCCUGGAAGAACCAUCACAGUGGGACCA |
| | | | | AGAGGAAAUGCAAGCAAUGCAGCACCUUCUG |
| | | | | CUUCUCCAAGAAAUGCUUCUGCUCCAAGAAC |
| | | | | CACCCCAACCCCUCCUCAGCCAAGAAAAGCAA |
| | | | | CCAAAUCCAAAGCAUCCACAGCAAAACCUGC |
| | | | | ACCUCCUCCAAAAACAGGACCUCCAAAAACC |
| | | | | UCCUCUGAACCUGUGAGAUGCAACAGACAUG |
| | | | | AUCCUCUGGCAAGAUAUGGAUCAAGAGUGCA |
| | | | | GAUCAGAUGCAGAUUUCCAAAUUCCACCAGA |
| | | | | ACAGAAUUCAGACUCCAGAUCUGGAGAUAUG |
| | | | | CAACAGCAACAGAUGCAGAAAUUGGAACAGC |
| | | | | ACCAUCUCUGGAAGAAGUGAUGGUGAAUGUG |
| | | | | UCUGCUCCUCCUGGAGGACAGCUGGUGUAUG |
| | | | | AUUCUGCUCCAAACAGAACAGAUCCUCAUGU |
| | | | | GAUCUGGGCUGAAGGAGCUGGACCUGGAGCU |
| | | | | UCUCCAAGACUGUACUCUGUGGUGGGACCUC |
| | | | | UGGGAAGACAGAGACUGAUCAUUGAAGAACU |
| | | | | GACCCUGGAAACCCAGGGAAUGUACUACUGG |
| | | | | GUGUGGGGAAGAACAGACAGACCUUCUGCUU |
| | | | | AUGGAACCUGGGUGAGAGUGAGAGUGUUCAG |
| | | | | ACCUCCUUCUCUGACCAUCCACCCUCAUGCUG |
| | | | | UGCUGGAAGGACAGCCUUUCAAAGCAACCUG |
| | | | | CACAGCAGCAACCUACUACCCUGGAAACAGA |
| | | | | GCUGAAUUUGUGUGGUUUGAAGAUGGAAGAA |
| | | | | GGGUGUUUGAUCCUGCUCAGAUCCACACCCA |
| | | | | GACCCAGGAAAAUCCUGAUGGAUUUUCCACA |
| | | | | GUGUCCACAGUGACAUCUGCUGCUGUGGGAG |
| | | | | GACAGGGACCUCCAAGAACCUUCACCUGCCA |
| | | | | GCUGACCUGGCACAGAGAUUCUGUGUCUUUU |
| | | | | UCAAGAAGAAAUGCUUCUGGAACAGCUUCUG |
| | | | | UGCUGCCAAGACCAACCAUCACCAUGGAAUU |
| | | | | CACAGGAGAUCAUGCUGUGUGCACAGCUGGA |
| | | | | UGUGUGCCUGAAGGAGUGACCUUUGCUUGGU |
| | | | | UUCUGGGAGAUGAUUCUUCUCCAGCUGAAAA |
| | | | | AGUGGCUGUGGCUUCCCAGACCUCUUGUGGA |
| | | | | AGACCUGGAACAGCAACCAUCAGAUCCACCC |
| | | | | UGCCUGUGUCUUAUGAACAGACAGAAUACAU |
| | | | | UUGCAGACUGGCUGGAUACCCUGAUGGAAUC |
| | | | | CCUGUGCUGGAACACCACUGAUAA |
| 200 | HSV-2 gI | HSV-2 | Version | AUGCCUGGGAGAAGCCUGCAAGGGCUCGCAA |
| | | gC | 4 | UCUUGGGCCUGUGGGUUUGUGCCACAGGCUU |
| | | | | GUCCCCGGGUCGAACAAUCACUGUUGGGCCC |
| | | | | AGGGGAAAUGCCAGCAAUGCUGCACCUUCAG |
| | | | | CAAGCCCACGAAACGCUUCAGCACCCAGGAC |
| | | | | AACACCCACUCCACCUCAACCGCGGAAAGCCA |
| | | | | CCAAGAGCAAGGCAAGUACCGCCAAACCCGC |
| | | | | UCCUCCUCCCAAGACAGGGCCACCCAAGACCU |
| | | | | CUAGUGAGCCAGUGAGGUGUAACCGCCAUGA |
| | | | | UCCCCUUGCCAGAUACGGGAGCAGAGUGCAG |
| | | | | AUUAGGUGCCGGUUUCCAAACUCCACGAGAA |
| | | | | CCGAAUUUCGCCUCCAGAUUUGGCGGUAUGC |
| | | | | GACUGCCACAGACGCAGAGAUUGGUACCGCU |
| | | | | CCCAGCCUGGAGGAGGUCAUGGUGAACGUGU |
| | | | | CAGCGCCUCCGGGUGGCCAGCUGGUCUACGA |
| | | | | CUCUGCCCCAAAUCGAACCGACCCUCACGUCA |
| | | | | UCUGGGCUGAAGGAGCGGGACCAGGAGCCUC |
| | | | | UCCACGCUUGUAUAGCGUAGUUGGCCCUCUG |
| | | | | GGGAGACAGCGCCUGAUCAUUGAGGAACUGA |
| | | | | CCCUUGAGACACAGGGGAUGUACUACUGGGU |
| | | | | GUGGGGCAGGACUGACAGGCCCAGUGCCUAU |
| | | | | GGAACUUGGGUUAGGGUCCGCGUCUUUCGGC |
| | | | | CACCCAGUCUGACCAUCCAUCCACAUGCCGUG |
| | | | | CUGGAAGGCCAGCCCUUCAAAGCGACUUGCA |
| | | | | CUGCCGCCACGUACUAUCCAGGGAAUAGAGC |
| | | | | CGAGUUCGUUUGGUUCGAGGAUGGCCGGAGA |
| | | | | GUAUUCGAUCCAGCUCAGAUCCACACCCAGA |
| | | | | CGCAGGAAAACCCGGACGGCUUUAGCACGGU |
| | | | | GAGUACCGUCACCUCUGCUGCCGUCGGAGGC |
| | | | | CAAGGACCUCCCCGUACCUUCACAUGCCAGCU |
| | | | | UACAUGGCACCGGGACUCAGUAAGCUUUUCA |
| | | | | CGUCGUAAUGCAUCCGGUACUGCUUCUGUGC |
| | | | | UGCCUCGACCCACCAUCACCAUGGAGUUCAC |
| | | | | AGGGGAUCACGCAGUGUGUACGGCAGGCUGC |
| | | | | GUGCCUGAAGGCGUGACAUUCGCCUGGUUUC |
| | | | | UCGGUGAUGACUCCUCUCCUGCUGAAAAGGU |
| | | | | GGCUGUAGCCUCCCAAACAAGCUGUGGUCGG |
| | | | | CCUGGAACUGCCACUAUACGCUCCACUCUCCC |
| | | | | GGUGUCCUACGAACAGACCGAGUACAUAUGC |
| | | | | AGACUGGCUGGAUAUCCCGAUGGCAUUCCCG |
| | | | | UGCUGGAGCAUCACUGA |
| 201 | HSV-2 | HSV-2 | Version | AUGGCUAGAGGUGCCGGCCUGGUGUUCUUUG |
| | gE | gC | 1 | UUGGCGUGUGGGUCGUGUCCUGUCUGGCCGC |
| | | | | UGCUCCUAGAACAUCUAGCCCUGGCAGAACC |
| | | | | AUCACAGUGGGCCCUAGAGGCAACGCCUCUA |
| | | | | AUGCCGCUCCUAGCGCCUCUCCUAGAAACGCC |
| | | | | UCUGCUCCCAGAACCACACCUACACCUCCACA |
| | | | | GCCUAGAAAGGCCACCAAGAGCAAGGCCAGC |
| | | | | ACAGCCAAACCUGCUCCUCCACCUAAGACAG |
| | | | | GCCCUCCAAAGACAAGCUCUGAGCCCGUGCG |
| | | | | GUGCAACAGACACGAUCCACUGGCCAGAUAC |
| | | | | GGCAGCCGGGUGCAGAUCAGAUGCAGAUUCC |
| | | | | CCAACAGCACCCGGACCGAGUUCCGGCUCCAG |
| | | | | AUUUGGAGAUACGCCACCGCCACAGAUGCCG |
| | | | | AGAUUGGAACAGCCCCUAGCCUGGAAGAAGU |
| | | | | GAUGGUCAACGUUUCAGCCCCUCCUGGCGGC |
| | | | | CAGCUGGUGUAUGAUUCUGCCCCUAACCGGA |
| | | | | CCGAUCCUCACGUGAUAUGGGCUGAAGGUGC |
| | | | | UGGCCCUGGCGCUUCCCCUAGACUGUAUUCU |
| | | | | GUUGUGGGCCCUCUGGGCAGACAGCGGCUGA |
| | | | | UCAUUGAGGAACUGACCCUGGAAACCCAGGG |
| | | | | CAUGUACUACUGGGUCUGGGGCAGAACCGAU |
| | | | | AGACCAAGCGCCUAUGGCACCUGGGUUCGAG |
| | | | | UGCGAGUGUUCAGACCUCCUAGCCUGACCAU |
| | | | | CCAUCCUCACGCCGUUCUGGAAGGCCAGCCU |
| | | | | UUCAAGGCCACAUGUACCGCCGCCACCUACU |
| | | | | AUCCCGGAAACAGAGCCGAGUUCGUUUGGUU |
| | | | | CGAGGACGGCAGAAGGGUGUUCGACCCCGCU |
| | | | | CAGAUCCACACACAGACCCAAGAGAACCCCG |
| | | | | ACGGCUUUAGCACCGUGUCCACAGUGACAUC |
| | | | | UGCCGCCGUUGGAGGACAGGGCCCUCCUAGA |
| | | | | ACCUUUACCUGCCAGCUGACCUGGCACAGAG |
| | | | | ACAGCGUGUCCUUCAGCAGAAGAAACGCCAG |
| | | | | CGGCACAGCCAGCGUUCUGCCUAGACCUACC |
| | | | | AUCACCAUGGAAUUCACCGGCGACCACGCCG |
| | | | | UGUGUACAGCUGGAUGUGUUCCUGAGGGCGU |
| | | | | GACCUUCGCUUGGUUUCUGGGCGACGAUAGC |
| | | | | AGCCCUGCCGAAAAAGUGGCUGUGGCCAGCC |
| | | | | AGACAAGCUGUGGCAGACCUGGAACCGCCAC |
| | | | | CAUCAGAAGCACACUGCCUGUCAGCUACGAG |
| | | | | CAGACCGAGUACAUCUGUCGGCUGGCCGGCU |
| | | | | AUCCUGAUGGCAUCCCUGUGCUGGAACACCA |
| | | | | CUGAUAA |
| 202 | HSV-2 | HSV-2 | Version | AUGGCGAGAGGAGCCGGGCUCGUGUUCUUUG |
| | gE | gC | 2 | UGGGCGUAUGGGUCGUUUCCUGCCUGGCUGC |
| | | | | CGCACCCAGGACCAGCAGCCCCGGCAGAACCA |
| | | | | UAACAGUAGGGCCACGGGGGAAUGCUUCCAA |
| | | | | UGCUGCACCUUCCGCUUCACCGAGGAAUGCU |
| | | | | UCUGCCCCAAGAACUACCCCCACUCCUCCUCA |
| | | | | ACCCAGGAAAGCGACAAAGUCCAAGGCCAGC |
| | | | | ACCGCAAAACCCGCUCCUCCUCCAAAGACUGG |
| | | | | GCCCCCAAAGACAAGUAGCGAACCAGUUCGG |
| | | | | UGCAACAGGCAUGACCCACUUGCACGCUAUG |
| | | | | GGUCAAGAGUCCAGAUACGGUGUCGCUUCCC |
| | | | | UAACAGUACAAGGACUGAGUUUCGGCUGCAG |
| | | | | AUCUGGCGUUAUGCCACAGCUACUGACGCAG |
| | | | | AGAUUGGUACCGCCCCCAGUUUGGAAGAGGU |
| | | | | GAUGGUCAACGUGUCCGCACCCCCAGGAGGA |
| | | | | CAGCUGGUCUAUGACUCAGCGCCCAAUAGGA |
| | | | | CCGAUCCCCACGUGAUCUGGGCAGAAGGAGC |
| | | | | CGGUCCUGGGGCCUCUCCACGGCUGUACUCA |
| | | | | GUUGUUGGCCCGCUUGGACGACAGAGACUCA |
| | | | | UCAUCGAAGAGCUGACACUGGAGACACAGGG |
| | | | | GAUGUACUACUGGGUGUGGGGCCGUACUGAC |
| | | | | CGCCCUUCCGCAUAUGGCACUUGGGUGAGAG |
| | | | | UUCGCGUCUUUCGGCCCCCUUCUCUCACCAUC |
| | | | | CAUCCUCAUGCCGUGCUCGAAGGCCAGCCCU |
| | | | | UUAAGGCCACAUGCACUGCUGCGACCUACUA |
| | | | | CCCUGGCAACAGAGCCGAGUUUGUCUGGUUU |
| | | | | GAGGAUGGUCGGCGAGUAUUCGAUCCAGCCC |
| | | | | AGAUUCACACACAAACGCAGGAAAAUCCGGA |
| | | | | CGGCUUCAGCACAGUGUCCACGGUGACCUCU |
| | | | | GCUGCAGUUGGUGGACAAGGACCCCCUCGAA |
| | | | | CCUUCACCUGUCAGCUGACCUGGCACAGAGA |
| | | | | CUCCGUAAGCUUCAGCCGUAGAAACGCCUCU |
| | | | | GGAACCGCCAGUGUGUUGCCGAGGCCGACUA |
| | | | | UCACGAUGGAAUUCACAGGCGAUCAUGCCGU |
| | | | | CUGUACUGCCGGCUGUGUGCCAGAAGGCGUA |
| | | | | ACCUUCGCUUGGUUUCUCGGGGAUGACUCAA |
| | | | | GUCCUGCAGAGAAAGUGGCUGUGGCCUCUCA |
| | | | | GACGAGCUGCGGUCGACCAGGAACAGCUACC |
| | | | | AUUCGCAGCACUCUGCCCGUGUCCUACGAGC |
| | | | | AGACGGAGUACAUCUGCAGGCUGGCCGGCUA |
| | | | | UCCCGAUGGGAUUCCAGUCCUGGAGCACCAC |
| | | | | UGAUAA |
| 351 | HSV-2 | HSV-2 | Version | AGCCCCGGCAGAACCAUAACAGUAGGGCCAC |
| | gE | gC | 2.1 | GGGGGAAUGCUUCCAAUGCUGCACCUUCCGC |
| | | | | UUCACCGAGGAAUGCUUCUGCCCCAAGAACU |
| | | | | ACCCCCACUCCUCCUCAACCCAGGAAAGCGAC |
| | | | | AAAGUCCAAGGCCAGCACCGCAAAACCCGCU |
| | | | | CCUCCUCCAAAGACUGGGCCCCCAAAGACAA |
| | | | | GUAGCGAACCAGUUCGGUGCAACAGGCAUGA |
| | | | | CCCACUUGCACGCUAUGGGUCAAGAGUCCAG |
| | | | | AUACGGUGUCGCUUCCCUAACAGUACAAGGA |
| | | | | CUGAGUUUCGGCUGCAGAUCUGGCGUUAUGC |
| | | | | CACAGCUACUGACGCAGAGAUUGGUACCGCC |
| | | | | CCCAGUUUGGAGGAAGUGAUGGUCAACGUGU |
| | | | | CCGCACCCCCAGGAGGACAGCUGGUCUAUGA |
| | | | | CUCAGCGCCCAAUAGGACCGAUCCCCACGUG |
| | | | | AUCUGGGCAGAAGGAGCCGGUCCUGGGGCCU |
| | | | | CUCCACGGCUGUACUCAGUUGUUGGCCCGCU |
| | | | | UGGACGACAGAGACUCAUCAUCGAGGAACUG |
| | | | | ACACUGGAGACACAGGGGAUGUACUACUGGG |
| | | | | UGUGGGGCCGUACUGACCGCCCUUCCGCAUA |
| | | | | UGGCACUUGGGUGAGAGUUCGCGUCUUUCGG |
| | | | | CCCCCUUCUCUCACCAUCCAUCCUCAUGCCGU |
| | | | | GCUCGAAGGCCAGCCCUUUAAGGCCACAUGC |
| | | | | ACUGCUGCGACCUACUACCCUGGCAACAGAG |
| | | | | CCGAGUUUGUCUGGUUUGAGGAUGGUCGGCG |
| | | | | AGUAUUCGAUCCAGCCCAGAUUCACACACAA |
| | | | | ACGCAGGAAAAUCCGGACGGCUUCAGCACAG |
| | | | | UGUCCACGGUGACCUCUGCUGCAGUUGGUGG |
| | | | | ACAAGGACCCCCUCGAACCUUCACCUGUCAGC |
| | | | | UGACCUGGCACAGAGACUCCGUAAGCUUCAG |
| | | | | CCGUAGAAACGCCUCUGGAACCGCCAGUGUG |
| | | | | UUGCCGAGGCCGACUAUCACGAUGGAAUUCA |
| | | | | CAGGCGAUCAUGCCGUCUGUACUGCCGGCUG |
| | | | | UGUGCCAGAAGGCGUAACCUUCGCUUGGUUU |
| | | | | CUCGGGGAUGACUCAAGUCCUGCAGAGAAAG |
| | | | | UGGCUGUGGCCUCUCAGACGAGCUGCGGUCG |
| | | | | ACCAGGAACAGCUACCAUUCGCAGCACUCUG |
| | | | | CCCGUGUCCUACGAGCAGACGGAGUACAUCU |
| | | | | GCAGGCUGGCCGGCUAUCCCGAUGGGAUUCC |
| | | | | AGUCCUGGAGCACCACUGAUAA |
| 203 | HSV-2 | HSV-2 | Version | AUGGCAAGAGGAGCAGGACUGGUGUUUUUCG |
| | gE | gC | 3 | UGGGAGUUUGGGUGGUGUCUUGCCUGGCUGC |
| | | | | UGCUCCAAGAACCUCUUCUCCUGGAAGAACC |
| | | | | AUCACAGUGGGACCAAGAGGAAAUGCAAGCA |
| | | | | AUGCAGCACCUUCUGCUUCUCCAAGAAAUGC |
| | | | | UUCUGCUCCAAGAACCACCCCAACCCCUCCUC |
| | | | | AGCCAAGAAAAGCAACCAAAUCCAAAGCAUC |
| | | | | CACAGCAAAACCUGCACCUCCUCCAAAAACA |
| | | | | GGACCUCCAAAAACCUCCUCUGAACCUGUGA |
| | | | | GAUGCAACAGACAUGAUCCUCUGGCAAGAUA |
| | | | | UGGAUCAAGAGUGCAGAUCAGAUGCAGAUUU |
| | | | | CCAAAUUCCACCAGAACAGAAUUCAGACUCC |
| | | | | AGAUCUGGAGAUAUGCAACAGCAACAGAUGC |
| | | | | AGAAAUUGGAACAGCACCAUCUCUGGAAGAA |
| | | | | GUGAUGGUGAAUGUGUCUGCUCCUCCUGGAG |
| | | | | GACAGCUGGUGUAUGAUUCUGCUCCAAACAG |
| | | | | AACAGAUCCUCAUGUGAUCUGGGCUGAAGGA |
| | | | | GCUGGACCUGGAGCUUCUCCAAGACUGUACU |
| | | | | CUGUGGUGGGACCUCUGGGAAGACAGAGACU |
| | | | | GAUCAUUGAAGAACUGACCCUGGAAACCCAG |
| | | | | GGAAUGUACUACUGGGUGUGGGGAAGAACAG |
| | | | | ACAGACCUUCUGCUUAUGGAACCUGGGUGAG |
| | | | | AGUGAGAGUGUUCAGACCUCCUUCUCUGACC |
| | | | | AUCCACCCUCAUGCUGUGCUGGAAGGACAGC |
| | | | | CUUUCAAAGCAACCUGCACAGCAGCAACCUA |
| | | | | CUACCCUGGAAACAGAGCUGAAUUUGUGUGG |
| | | | | UUUGAAGAUGGAAGAAGGGUGUUUGAUCCUG |
| | | | | CUCAGAUCCACACCCAGACCCAGGAAAAUCC |
| | | | | UGAUGGAUUUUCCACAGUGUCCACAGUGACA |
| | | | | UCUGCUGCUGUGGGAGGACAGGGACCUCCAA |
| | | | | GAACCUUCACCUGCCAGCUGACCUGGCACAG |
| | | | | AGAUUCUGUGUCUUUUUCAAGAAGAAAUGCU |
| | | | | UCUGGAACAGCUUCUGUGCUGCCAAGACCAA |
| | | | | CCAUCACCAUGGAAUUCACAGGAGAUCAUGC |
| | | | | UGUGUGCACAGCUGGAUGUGUGCCUGAAGGA |
| | | | | GUGACCUUUGCUUGGUUUCUGGGAGAUGAUU |
| | | | | CUUCUCCAGCUGAAAAAGUGGCUGUGGCUUC |
| | | | | CCAGACCUCUUGUGGAAGACCUGGAACAGCA |
| | | | | ACCAUCAGAUCCACCCUGCCUGUGUCUUAUG |
| | | | | AACAGACAGAAUACAUUUGCAGACUGGCUGG |
| | | | | AUACCCUGAUGGAAUCCCUGUGCUGGAACAC |
| | | | | CACUGAUAA |
| 204 | HSV-2 | HSV-2 | Version | AUGGCGAGAGGAGCCGGACUCGUGUUCUUUG |
| | gE | gC | 4 | UGGGAGUCUGGGUUGUGAGCUGCCUGGCAGC |
| | | | | AGCUCCACGCACUAGCUCUCCAGGCAGAACU |
| | | | | AUCACAGUGGGACCCAGAGGGAAUGCCAGCA |
| | | | | AUGCAGCCCCGAGUGCCAGCCCUCGUAACGCC |
| | | | | AGCGCUCCCAGAACAACCCCAACUCCACCGCA |
| | | | | GCCUAGAAAGGCGACCAAGUCCAAAGCAUCC |
| | | | | ACUGCAAAACCAGCCCCACCUCCCAAAACGGG |
| | | | | ACCUCCCAAGACCAGCUCCGAGCCUGUAAGG |
| | | | | UGCAAUCGGCAUGACCCCUUGGCCCGAUAUG |
| | | | | GCAGUCGCGUGCAGAUUCGAUGUCGGUUUCC |
| | | | | CAACUCUACCCGGACUGAGUUCCGGUUGCAG |
| | | | | AUCUGGAGGUAUGCGACCGCCACUGACGCUG |
| | | | | AGAUCGGCACAGCACCAAGCCUGGAAGAAGU |
| | | | | GAUGGUGAACGUUAGUGCUCCUCCGGGCGGG |
| | | | | CAACUCGUGUAUGACUCCGCACCCAACCGCAC |
| | | | | AGAUCCUCACGUGAUUUGGGCCGAAGGAGCC |
| | | | | GGACCCGGUGCGUCACCUAGGCUCUACUCUG |
| | | | | UCGUAGGACCACUGGGCCGUCAACGCCUGAU |
| | | | | AAUCGAGGAGCUGACUCUGGAGACACAGGGU |
| | | | | AUGUACUACUGGGUCUGGGGCAGAACCGACA |
| | | | | GGCCAUCUGCUUACGGGACAUGGGUCCGCGU |
| | | | | UCGAGUAUUUCGGCCACCCUCACUGACCAUA |
| | | | | CAUCCCCAUGCCGUUCUUGAAGGGCAGCCUU |
| | | | | UCAAGGCAACCUGUACUGCUGCCACAUACUA |
| | | | | UCCCGGGAAUAGGGCCGAGUUCGUCUGGUUU |
| | | | | GAAGAUGGCCGAAGGGUGUUUGACCCGGCUC |
| | | | | AGAUCCACACCCAGACACAGGAGAACCCCGA |
| | | | | UGGCUUCAGUACGGUGUCUACCGUCACAAGC |
| | | | | GCCGCUGUGGGUGGCCAAGGUCCUCCCAGAA |
| | | | | CUUUCACCUGUCAGCUGACGUGGCACAGGGA |
| | | | | UUCCGUGAGCUUUUCCCGCCGCAAUGCGUCA |
| | | | | GGGACCGCCUCCGUGCUUCCUCGGCCAACCAU |
| | | | | CACAAUGGAAUUCACGGGUGAUCACGCUGUC |
| | | | | UGCACAGCUGGCUGCGUUCCUGAGGGCGUGA |
| | | | | CAUUCGCAUGGUUUCUUGGUGACGACUCAUC |
| | | | | UCCCGCAGAGAAGGUGGCUGUUGCCUCACAA |
| | | | | ACGAGUUGUGGGCGUCCAGGCACUGCCACCA |
| | | | | UUCGGUCCACCCUCCCCGUAAGCUACGAACA |
| | | | | GACUGAGUAUAUUUGCAGACUGGCUGGAUAC |
| | | | | CCGGAUGGGAUUCCUGUCCUGGAACAUCACU |
| | | | | GA |
| 205 | EboZ | HSV-2 | Version | AUGGGAGUGACCGGCAUUCUCCAGCUGCCUC |
| | | gC | 1 | GGGACAGAUUCAAGCGGACCAGCUUCUUCCU |
| | | | | GUGGGUCAUCAUCCUGUUCCAGCGGACCUUC |
| | | | | AGCAUCCCCAGCCCUGGCAGAACCAUCACAG |
| | | | | UGGGCCCUAGAGGCAACGCCUCUAAUGCCGC |
| | | | | UCCUAGCGCCUCUCCUAGAAACGCCUCUGCUC |
| | | | | CCAGAACCACACCUACACCUCCACAGCCUAGA |
| | | | | AAGGCCACCAAGAGCAAGGCCAGCACAGCCA |
| | | | | AACCUGCUCCUCCACCUAAGACAGGCCCUCCA |
| | | | | AAGACAAGCUCUGAGCCCGUGCGGUGCAACA |
| | | | | GACACGAUCCACUGGCCAGAUACGGCAGCCG |
| | | | | GGUGCAGAUCAGAUGCAGAUUCCCCAACAGC |
| | | | | ACCCGGACCGAGUUCCGGCUCCAGAUUUGGA |
| | | | | GAUACGCCACCGCCACAGAUGCCGAGAUUGG |
| | | | | AACAGCCCCUAGCCUGGAAGAAGUGAUGGUC |
| | | | | AACGUUUCAGCCCCUCCUGGCGGCCAGCUGG |
| | | | | UGUAUGAUUCUGCCCCUAACCGGACCGAUCC |
| | | | | UCACGUGAUAUGGGCUGAAGGUGCUGGCCCU |
| | | | | GGCGCUUCCCCUAGACUGUAUUCUGUUGUGG |
| | | | | GCCCUCUGGGCAGACAGCGGCUGAUCAUUGA |
| | | | | GGAACUGACCCUGGAAACCCAGGGCAUGUAC |
| | | | | UACUGGGUCUGGGGCAGAACCGAUAGACCAA |
| | | | | GCGCCUAUGGCACCUGGGUUCGAGUGCGAGU |
| | | | | GUUCAGACCUCCUAGCCUGACCAUCCAUCCUC |
| | | | | ACGCCGUUCUGGAAGGCCAGCCUUUCAAGGC |
| | | | | CACAUGUACCGCCGCCACCUACUAUCCCGGAA |
| | | | | ACAGAGCCGAGUUCGUUUGGUUCGAGGACGG |
| | | | | CAGAAGGGUGUUCGACCCCGCUCAGAUCCAC |
| | | | | ACACAGACCCAAGAGAACCCCGACGGCUUUA |
| | | | | GCACCGUGUCCACAGUGACAUCUGCCGCCGU |
| | | | | UGGAGGACAGGGCCCUCCUAGAACCUUUACC |
| | | | | UGCCAGCUGACCUGGCACAGAGACAGCGUGU |
| | | | | CCUUCAGCAGAAGAAACGCCAGCGGCACAGC |
| | | | | CAGCGUUCUGCCUAGACCUACCAUCACCAUG |
| | | | | GAAUUCACCGGCGACCACGCCGUGUGUACAG |
| | | | | CUGGAUGUGUUCCUGAGGGCGUGACCUUCGC |
| | | | | UUGGUUUCUGGGCGACGAUAGCAGCCCUGCC |
| | | | | GAAAAAGUGGCUGUGGCCAGCCAGACAAGCU |
| | | | | GUGGCAGACCUGGAACCGCCACCAUCAGAAG |
| | | | | CACACUGCCUGUCAGCUACGAGCAGACCGAG |
| | | | | UACAUCUGUCGGCUGGCCGGCUAUCCUGAUG |
| | | | | GCAUCCCUGUGCUGGAACACCACUGAUAA |
| 206 | EboZ | HSV-2 | Version | AUGGGAGUGACUGGCAUACUCCAGCUUCCUA |
| | | gC | 2 | GAGACAGGUUUAAGCGCACAUCCUUCUUUCU |
| | | | | GUGGGUCAUCAUCCUGUUCCAACGGACCUUC |
| | | | | AGCAUUCCCAGCCCCGGCAGAACCAUAACAG |
| | | | | UAGGGCCACGGGGGAAUGCUUCCAAUGCUGC |
| | | | | ACCUUCCGCUUCACCGAGGAAUGCUUCUGCC |
| | | | | CCAAGAACUACCCCCACUCCUCCUCAACCCAG |
| | | | | GAAAGCGACAAAGUCCAAGGCCAGCACCGCA |
| | | | | AAACCCGCUCCUCCUCCAAAGACUGGGCCCCC |
| | | | | AAAGACAAGUAGCGAACCAGUUCGGUGCAAC |
| | | | | AGGCAUGACCCACUUGCACGCUAUGGGUCAA |
| | | | | GAGUCCAGAUACGGUGUCGCUUCCCUAACAG |
| | | | | UACAAGGACUGAGUUUCGGCUGCAGAUCUGG |
| | | | | CGUUAUGCCACAGCUACUGACGCAGAGAUUG |
| | | | | GUACCGCCCCCAGUUUGGAAGAGGUGAUGGU |
| | | | | CAACGUGUCCGCACCCCCAGGAGGACAGCUG |
| | | | | GUCUAUGACUCAGCGCCCAAUAGGACCGAUC |
| | | | | CCCACGUGAUCUGGGCAGAAGGAGCCGGUCC |
| | | | | UGGGGCCUCUCCACGGCUGUACUCAGUUGUU |
| | | | | GGCCCGCUUGGACGACAGAGACUCAUCAUCG |
| | | | | AAGAGCUGACACUGGAGACACAGGGGAUGUA |
| | | | | CUACUGGGUGUGGGGCCGUACUGACCGCCCU |
| | | | | UCCGCAUAUGGCACUUGGGUGAGAGUUCGCG |
| | | | | UCUUUCGGCCCCCUUCUCUCACCAUCCAUCCU |
| | | | | CAUGCCGUGCUCGAAGGCCAGCCCUUUAAGG |
| | | | | CCACAUGCACUGCUGCGACCUACUACCCUGGC |
| | | | | AACAGAGCCGAGUUUGUCUGGUUUGAGGAUG |
| | | | | GUCGGCGAGUAUUCGAUCCAGCCCAGAUUCA |
| | | | | CACACAAACGCAGGAAAAUCCGGACGGCUUC |
| | | | | AGCACAGUGUCCACGGUGACCUCUGCUGCAG |
| | | | | UUGGUGGACAAGGACCCCCUCGAACCUUCAC |
| | | | | CUGUCAGCUGACCUGGCACAGAGACUCCGUA |
| | | | | AGCUUCAGCCGUAGAAACGCCUCUGGAACCG |
| | | | | CCAGUGUGUUGCCGAGGCCGACUAUCACGAU |
| | | | | GGAAUUCACAGGCGAUCAUGCCGUCUGUACU |
| | | | | GCCGGCUGUGUGCCAGAAGGCGUAACCUUCG |
| | | | | CUUGGUUUCUCGGGGAUGACUCAAGUCCUGC |
| | | | | AGAGAAAGUGGCUGUGGCCUCUCAGACGAGC |
| | | | | UGCGGUCGACCAGGAACAGCUACCAUUCGCA |
| | | | | GCACUCUGCCCGUGUCCUACGAGCAGACGGA |
| | | | | GUACAUCUGCAGGCUGGCCGGCUAUCCCGAU |
| | | | | GGGAUUCCAGUCCUGGAGCACCACUGAUAA |
| 207 | EboZ | HSV-2 | Version | AUGGGAGUAACCGGAAUUCUCCAGCUGCCAA |
| | | gC | 3 | GAGAUCGAUUCAAAAGAACAUCAUUUUUCCU |
| | | | | UUGGGUAAUUAUUCUGUUUCAGAGAACAUUU |
| | | | | UCCAUCCCUUCUCCUGGAAGAACCAUCACAG |
| | | | | UGGGACCAAGAGGAAAUGCAAGCAAUGCAGC |
| | | | | ACCUUCUGCUUCUCCAAGAAAUGCUUCUGCU |
| | | | | CCAAGAACCACCCCAACCCCUCCUCAGCCAAG |
| | | | | AAAAGCAACCAAAUCCAAAGCAUCCACAGCA |
| | | | | AAACCUGCACCUCCUCCAAAAACAGGACCUCC |
| | | | | AAAAACCUCCUCUGAACCUGUGAGAUGCAAC |
| | | | | AGACAUGAUCCUCUGGCAAGAUAUGGAUCAA |
| | | | | GAGUGCAGAUCAGAUGCAGAUUUCCAAAUUC |
| | | | | CACCAGAACAGAAUUCAGACUCCAGAUCUGG |
| | | | | AGAUAUGCAACAGCAACAGAUGCAGAAAUUG |
| | | | | GAACAGCACCAUCUCUGGAAGAAGUGAUGGU |
| | | | | GAAUGUGUCUGCUCCUCCUGGAGGACAGCUG |
| | | | | GUGUAUGAUUCUGCUCCAAACAGAACAGAUC |
| | | | | CUCAUGUGAUCUGGGCUGAAGGAGCUGGACC |
| | | | | UGGAGCUUCUCCAAGACUGUACUCUGUGGUG |
| | | | | GGACCUCUGGGAAGACAGAGACUGAUCAUUG |
| | | | | AAGAACUGACCCUGGAAACCCAGGGAAUGUA |
| | | | | CUACUGGGUGUGGGGAAGAACAGACAGACCU |
| | | | | UCUGCUUAUGGAACCUGGGUGAGAGUGAGAG |
| | | | | UGUUCAGACCUCCUUCUCUGACCAUCCACCCU |
| | | | | CAUGCUGUGCUGGAAGGACAGCCUUUCAAAG |
| | | | | CAACCUGCACAGCAGCAACCUACUACCCUGG |
| | | | | AAACAGAGCUGAAUUUGUGUGGUUUGAAGAU |
| | | | | GGAAGAAGGGUGUUUGAUCCUGCUCAGAUCC |
| | | | | ACACCCAGACCCAGGAAAAUCCUGAUGGAUU |
| | | | | UUCCACAGUGUCCACAGUGACAUCUGCUGCU |
| | | | | GUGGGAGGACAGGGACCUCCAAGAACCUUCA |
| | | | | CCUGCCAGCUGACCUGGCACAGAGAUUCUGU |
| | | | | GUCUUUUUCAAGAAGAAAUGCUUCUGGAACA |
| | | | | GCUUCUGUGCUGCCAAGACCAACCAUCACCA |
| | | | | UGGAAUUCACAGGAGAUCAUGCUGUGUGCAC |
| | | | | AGCUGGAUGUGUGCCUGAAGGAGUGACCUUU |
| | | | | GCUUGGUUUCUGGGAGAUGAUUCUUCUCCAG |
| | | | | CUGAAAAAGUGGCUGUGGCUUCCCAGACCUC |
| | | | | UUGUGGAAGACCUGGAACAGCAACCAUCAGA |
| | | | | UCCACCCUGCCUGUGUCUUAUGAACAGACAG |
| | | | | AAUACAUUUGCAGACUGGCUGGAUACCCUGA |
| | | | | UGGAAUCCCUGUGCUGGAACACCACUGAUAA |
| 208 | EboZ | HSV-2 | Version | AUGGGAGUUACCGGAAUUCUGCAAUUGCCCA |
| | | gC | 4 | GGGAUCGGUUCAAGCGCACAUCCUUCUUCCU |
| | | | | GUGGGUCAUCAUCCUCUUUCAGCGUACUUUC |
| | | | | UCCAUACCCUCUCCCGGACGAACUAUCACUG |
| | | | | UAGGCCCAAGGGGCAACGCUAGUAAUGCCGC |
| | | | | UCCCAGUGCUUCACCACGCAAUGCGAGCGCA |
| | | | | CCCAGAACUACACCCACACCUCCUCAGCCGAG |
| | | | | GAAAGCCACCAAGUCCAAAGCCAGCACCGCC |
| | | | | AAACCCGCUCCUCCACCUAAAACAGGGCCUCC |
| | | | | CAAGACCUCAAGCGAGCCCGUUAGAUGCAAU |
| | | | | CGGCAUGAUCCACUGGCUCGUUACGGUUCUC |
| | | | | GGGUCCAGAUACGCUGUAGGUUUCCUAACUC |
| | | | | CACACGAACCGAGUUCAGAUUGCAGAUCUGG |
| | | | | AGAUAUGCCACAGCCACUGACGCUGAGAUUG |
| | | | | GCACUGCACCUAGUCUGGAGGAGGUGAUGGU |
| | | | | GAACGUGAGCGCCCCUCCUGGCGGUCAGCUU |
| | | | | GUGUAUGACUCAGCACCGAAUCGCACAGAUC |
| | | | | CGCACGUCAUAUGGGCCGAAGGUGCAGGGCC |
| | | | | CGGUGCAUCCCCUCGGCUGUAUUCCGUGGUC |
| | | | | GGACCACUCGGGCGCCAGAGGCUUAUCAUUG |
| | | | | AGGAACUGACCCUCGAAACCCAGGGUAUGUA |
| | | | | CUACUGGGUAUGGGGCCGUACCGACCGGCCU |
| | | | | AGCGCCUACGGAACUUGGGUGAGAGUUCGGG |
| | | | | UGUUCAGACCGCCAAGUCUUACAAUUCACCC |
| | | | | UCAUGCCGUGCUCGAAGGUCAGCCAUUUAAG |
| | | | | GCCACGUGUACUGCGGCUACGUACUAUCCCG |
| | | | | GGAAUCGGGCUGAAUUCGUGUGGUUUGAGGA |
| | | | | UGGCAGGAGAGUGUUCGACCCAGCCCAAAUC |
| | | | | CACACCCAAACACAGGAGAACCCAGACGGGU |
| | | | | UUUCCACCGUGUCAACGGUCACAUCUGCCGC |
| | | | | CGUCGGAGGACAAGGGCCACCCAGAACCUUC |
| | | | | ACAUGCCAGCUGACCUGGCAUAGGGAUAGCG |
| | | | | UAAGCUUUAGCCGGCGAAACGCAUCUGGAAC |
| | | | | GGCGAGCGUUCUGCCUCGACCAACAAUCACC |
| | | | | AUGGAGUUCACCGGCGAUCACGCAGUGUGCA |
| | | | | CUGCUGGGUGUGUACCCGAAGGCGUGACAUU |
| | | | | UGCCUGGUUUCUGGGAGAUGACUCCUCACCC |
| | | | | GCAGAAAAGGUCGCCGUUGCAUCUCAGACCA |
| | | | | GUUGUGGCAGGCCCGGGACUGCUACCAUCCG |
| | | | | CAGCACUCUGCCGGUGUCUUACGAACAGACG |
| | | | | GAGUACAUUUGCCGCUUGGCGGGCUAUCCAG |
| | | | | ACGGCAUUCCAGUUCUGGAGCAUCACUGA |
| 116 | HSV-2 | HSV-2 | | AUGGGCCGCCUGACCUCCGGCGUGGGCACCG |
| | gD | gD | | CCGCCCUGCUGGUGGUGGCCGUGGGCCUGCG |
| | | | | CGUGGUGUGCGCCAAGUACGCCCUGGCCGAC |
| | | | | CCCUCCCUGAAGAUGGCCGACCCCAACCGCUU |
| | | | | CCGCGGCAAGAACCUGCCCGUGCUGGACCAG |
| | | | | CUGACCGACCCCCCCGGCGUGAAGCGCGUGU |
| | | | | ACCACAUCCAGCCCUCCCUGGAGGACCCCUUC |
| | | | | CAGCCCCCCUCCAUCCCCAUCACCGUGUACUA |
| | | | | CGCCGUGCUGGAGCGCGCCUGCCGCUCCGUGC |
| | | | | UGCUGCACGCCCCCUCCGAGGCCCCCCAGAUC |
| | | | | GUGCGCGGCGCCUCCGACGAGGCCCGCAAGC |
| | | | | ACACCUACAACCUGACCAUCGCCUGGUACCGC |
| | | | | AUGGGCGACAACUGCGCCAUCCCCAUCACCG |
| | | | | UGAUGGAGUACACCGAGUGCCCCUACAACAA |
| | | | | GUCCCUGGGCGUGUGCCCCAUCCGCACCCAGC |
| | | | | CCCGCUGGUCCUACUACGACUCCUUCUCCGCC |
| | | | | GUGUCCGAGGACAACCUGGGCUUCCUGAUGC |
| | | | | ACGCCCCCGCCUUCGAGACCGCCGGCACCUAC |
| | | | | CUGCGCCUGGUGAAGAUCAACGACUGGACCG |
| | | | | AGAUCACCCAGUUCAUCCUGGAGCACCGCGC |
| | | | | CCGCGCCUCCUGCAAGUACGCCCUGCCCCUGC |
| | | | | GCAUCCCCCCCGCCGCCUGCCUGACCUCCAAG |
| | | | | GCCUACCAGCAGGGCGUGACCGUGGACUCCA |
| | | | | UCGGCAUGCUGCCCCGCUUCAUCCCCGAGAAC |
| | | | | CAGCGCACCGUGGCCCUGUACUCCCUGAAGA |
| | | | | UCGCCGGCUGGCACGGCCCCAAGCCCCCCUAC |
| | | | | ACCUCCACCCUGCUGCCCCCCGAGCUGUCCGA |
| | | | | CACCACCAACGCCACCCAGCCCGAGCUGGUGC |
| | | | | CCGAGGACCCCGAGGACUCCGCCCUGCUGGA |
| | | | | GGACCCCGCCGGCACCGUGUCCUCCCAGAUCC |
| | | | | CCCCCAACUGGCACAUCCCCUCCAUCCAGGAC |
| | | | | GUGGCCCCCCACCACUAA |
| 117 | HSV-2 | HSV-2 | Version | AUGGGCAGACUGACAUCUGGCGUGGGAACAG |
| | gD | gD | 1 | CUGCUCUGCUGGUGGUUGCUGUGGGCCUGAG |
| | | | | AGUCGUGUGUGCCAAAUACGCCCUGGCCGAU |
| | | | | CCUAGCCUGAAGAUGGCUGACCCCAACCGGU |
| | | | | UCCGGGGCAAGAAUCUGCCUGUUCUGGACCA |
| | | | | GCUGACCGAUCCUCCUGGCGUGAAACGGGUG |
| | | | | UACCACAUCCAGCCAAGCCUGGAAGAUCCCU |
| | | | | UCCAGCCUCCUAGCAUCCCCAUCACCGUGUAC |
| | | | | UACGCCGUGCUGGAAAGGGCCUGUAGAAGCG |
| | | | | UGCUGCUGCACGCCCCAUCUGAAGCCCCUCAA |
| | | | | AUCGUCAGAGGCGCUUCCGACGAGGCCAGAA |
| | | | | AGCACACCUACAACCUGACAAUCGCCUGGUA |
| | | | | CAGAAUGGGCGACAACUGCGCCAUUCCUAUC |
| | | | | ACCGUGAUGGAGUACACCGAGUGUCCCUACA |
| | | | | ACAAGAGCCUGGGCGUGUGCCCCAUCAGAAC |
| | | | | ACAGCCUAGAUGGUCCUACUACGACAGCUUC |
| | | | | AGCGCCGUGUCCGAGGACAAUCUGGGCUUCC |
| | | | | UGAUGCAUGCCCCUGCCUUUGAGACAGCCGG |
| | | | | CACCUAUCUGCGGCUGGUCAAGAUCAACGAC |
| | | | | UGGACCGAGAUCACCCAGUUCAUCCUGGAAC |
| | | | | ACAGAGCCAGAGCCAGCUGCAAAUACGCUCU |
| | | | | GCCCCUGAGAAUUCCUCCUGCCGCCUGUCUG |
| | | | | ACAAGCAAGGCCUAUCAGCAGGGCGUGACCG |
| | | | | UGGAUAGCAUCGGCAUGCUGCCCAGAUUCAU |
| | | | | CCCCGAGAACCAGAGAACAGUGGCCCUGUAC |
| | | | | UCCCUGAAGAUCGCCGGAUGGCACGGACCCA |
| | | | | AGCCUCCAUACACAAGCACACUGCUGCCUCCA |
| | | | | GAGCUGAGCGACACCACCAAUGCCACACAGC |
| | | | | CUGAACUGGUGCCUGAGGACCCAGAGGAUUC |
| | | | | UGCCCUGCUUGAAGAUCCUGCCGGCACCGUG |
| | | | | UCUAGCCAGAUUCCUCCUAACUGGCACAUCC |
| | | | | CCAGCAUCCAGGAUGUGGCCCCUCAUCAUUG |
| | | | | A |
| 118 | HSV-2 | HSV-2 | Version | AUGGGGAGACUCACAUCAGGCGUAGGAACCG |
| | gD | gD | 2 | CUGCCCUGUUGGUCGUGGCCGUUGGUCUGAG |
| | | | | AGUUGUGUGUGCCAAAUAUGCUCUCGCUGAU |
| | | | | CCGAGCCUCAAGAUGGCAGAUCCCAACCGAU |
| | | | | UUCGGGGAAAGAAUCUGCCAGUACUGGACCA |
| | | | | GCUGACGGACCCACCUGGCGUCAAACGCGUC |
| | | | | UACCACAUACAGCCUAGUCUUGAGGACCCUU |
| | | | | UUCAGCCACCGUCUAUCCCCAUUACCGUGUA |
| | | | | CUAUGCCGUGCUGGAACGCGCGUGUAGGUCA |
| | | | | GUUCUGCUGCAUGCCCCAUCCGAAGCCCCCCA |
| | | | | GAUCGUCAGAGGAGCUUCUGAUGAAGCACGC |
| | | | | AAACACACCUACAACCUCACAAUAGCGUGGU |
| | | | | AUCGAAUGGGCGAUAACUGCGCAAUUCCCAU |
| | | | | CACAGUCAUGGAGUACACGGAGUGCCCCUAC |
| | | | | AACAAGAGCCUCGGUGUUUGCCCUAUCAGGA |
| | | | | CACAACCCAGGUGGAGCUAUUACGACAGUUU |
| | | | | CAGCGCCGUGUCUGAGGACAAUCUGGGGUUU |
| | | | | CUGAUGCACGCACCCGCCUUCGAGACUGCCG |
| | | | | GCACCUACUUGCGGCUGGUGAAGAUCAACGA |
| | | | | CUGGACUGAGAUCACCCAGUUCAUCCUGGAA |
| | | | | CAUAGGGCCAGAGCCAGCUGCAAGUAUGCUC |
| | | | | UUCCCCUGCGGAUUCCGCCUGCAGCAUGUCU |
| | | | | GACCUCAAAAGCCUACCAGCAAGGGGUGACU |
| | | | | GUGGACAGCAUUGGCAUGCUGCCUCGUUUCA |
| | | | | UUCCCGAGAAUCAACGGACAGUGGCUCUGUA |
| | | | | UUCCCUGAAGAUCGCAGGAUGGCAUGGGCCC |
| | | | | AAACCACCUUAUACCUCUACGUUGCUUCCAC |
| | | | | CAGAACUCAGUGACACCACUAAUGCGACACA |
| | | | | GCCAGAACUUGUGCCUGAGGAUCCUGAAGAU |
| | | | | AGCGCUCUGUUGGAGGAUCCAGCCGGUACUG |
| | | | | UGUCCUCCCAGAUACCACCCAAUUGGCACAU |
| | | | | UCCUUCCAUUCAGGACGUAGCUCCGCAUCAC |
| | | | | UGA |
| 119 | HSV-2 | HSV-2 | Version | AUGGGGAGACUCACAUCAGGCGUAGGAACCG |
| | gD | gD | 2.1 | CUGCCCUGUUGGUCGUGGCCGUUGGUCUGAG |
| | | | | AGUUGUGUGUGCCAAAUAUGCUCUCGCUGAU |
| | | | | CCGAGCCUCAAGAUGGCAGAUCCCAACCGAU |
| | | | | UUCGGGGAAAGAAUCUGCCAGUACUGGACCA |
| | | | | GCUGACGGACCCACCUGGCGUCAAACGCGUC |
| | | | | UACCACAUACAGCCUAGUCUUGAGGACCCUU |
| | | | | UUCAGCCACCGUCUAUCCCCAUUACCGUGUA |
| | | | | CUAUGCCGUGCUGGAACGCGCGUGUAGGUCA |
| | | | | GUUCUGCUGCAUGCCCCAUCCGAAGCCCCCCA |
| | | | | GAUCGUCAGAGGAGCUUCUGAUGAAGCACGC |
| | | | | AAACACACCUACAACCUCACAAUAGCGUGGU |
| | | | | AUCGAAUGGGCGAUAACUGCGCAAUUCCCAU |
| | | | | CACAGUCAUGGAGUACACGGAGUGCCCCUAC |
| | | | | AACAAGAGCCUCGGUGUUUGCCCUAUCAGGA |
| | | | | CACAACCCAGGUGGAGCUAUUACGACAGUUU |
| | | | | CAGCGCCGUGUCUGAGGACAAUCUGGGGUUU |
| | | | | CUGAUGCACGCACCCGCCUUCGAGACUGCCG |
| | | | | GCACCUACUUGCGGCUGGUGAAGAUCAACGA |
| | | | | CUGGACUGAGAUCACCCAGUUCAUCCUGGAA |
| | | | | CAUAGGGCCAGAGCCAGCUGCAAGUAUGCCC |
| | | | | UUCCCCUGCGGAUUCCGCCUGCAGCAUGUCU |
| | | | | GACCUCAAAAGCCUACCAGCAAGGGGUGACU |
| | | | | GUGGACAGCAUUGGCAUGCUGCCUCGUUUCA |
| | | | | UUCCCGAGAAUCAACGGACAGUGGCUCUGUA |
| | | | | UUCCCUGAAGAUCGCAGGAUGGCAUGGGCCC |
| | | | | AAACCACCUUAUACCUCUACGUUGCUUCCAC |
| | | | | CAGAACUCAGUGACACCACUAAUGCGACACA |
| | | | | GCCAGAACUUGUGCCUGAGGAUCCUGAAGAU |
| | | | | AGCGCUCUGUUGGAGGAUCCAGCCGGUACUG |
| | | | | UGUCCUCCCAGAUACCACCCAAUUGGCACAU |
| | | | | UCCUUCCAUUCAGGACGUAGCUCCGCAUCAC |
| | | | | UGA |
| 352 | HSV-2 | HSV-2 | Version | AUGGGGAGACUCACAUCAGGCGUAGGAACCG |
| | gD | gD | 2.2 | CUGCCCUGUUGGUCGUGGCCGUUGGUCUGAG |
| | | | | AGUUGUGUGUGCCAAAUAUGCUCUCGCUGAU |
| | | | | CCGAGCCUCAAGAUGGCAGAUCCCAACCGAU |
| | | | | UUCGGGGAAAGAAUCUGCCAGUACUGGACCA |
| | | | | GCUGACGGACCCACCUGGCGUCAAACGCGUC |
| | | | | UACCACAUACAGCCUAGUCUUGAGGACCCUU |
| | | | | UUCAGCCACCGUCUAUCCCCAUUACCGUGUA |
| | | | | CUAUGCCGUGCUGGAACGCGCGUGUAGGUCA |
| | | | | GUUCUGCUGCAUGCCCCAUCCGAAGCCCCCCA |
| | | | | GAUCGUCAGAGGAGCUUCUGAUGAAGCACGC |
| | | | | AAACACACCUACAACCUCACAAUAGCGUGGU |
| | | | | AUCGAAUGGGCGAUAACUGCGCAAUUCCCAU |
| | | | | CACAGUCAUGGAGUACACGGAGUGCCCCUAC |
| | | | | AACAAGAGCCUCGGUGUUUGCCCUAUCAGGA |
| | | | | CACAACCCAGGUGGAGCUAUUACGACAGUUU |
| | | | | CAGCGCCGUGUCUGAGGACAAUCUGGGGUUU |
| | | | | CUGAUGCACGCACCCGCCUUCGAGACUGCCG |
| | | | | GCACCUACUUGCGGCUGGUGAAGAUCAACGA |
| | | | | CUGGACUGAGAUCACCCAGUUCAUCCUGGAA |
| | | | | CAUAGGGCCAGAGCCAGCUGCAAGUAUGCCC |
| | | | | UUCCCCUGCGGAUUCCGCCUGCAGCAUGUCU |
| | | | | GACCUCAAAAGCCUACCAGCAAGGGGUGACU |
| | | | | GUGGACAGCAUUGGCAUGCUGCCUCGUUUCA |
| | | | | UUCCCGAGAAUCAACGGACAGUGGCUCUGUA |
| | | | | UUCCCUGAAGAUCGCAGGAUGGCAUGGGCCC |
| | | | | AAACCACCUUAUACCUCUACGUUGCUUCCAC |
| | | | | CAGAACUCAGUGACACCACUAAUGCGACACA |
| | | | | GCCAGAACUUGUGCCUGAGGAUCCUGAAGAU |
| | | | | AGCGCUCUGUUGGAGGAUCCAGCCGGUACUG |
| | | | | UGUCCUCCCAGAUACCACCCAAUUGGCACAU |
| | | | | UCCUUCCAUUCAGGACGUAGCUCCGCAUCAC |
| | | | | UGAUAA |
| 120 | HSV-2 | HSV-2 | Version | AUGGGCAGACUGACCUCCGGCGUGGGCACCG |
| | gD | gD | 3 | CCGCCCUGCUGGUGGUGGCCGUGGGCCUGAG |
| | | | | AGUGGUGUGCGCCAAAUACGCCCUGGCCGAU |
| | | | | CCCUCCCUGAAAAUGGCCGAUCCCAACAGGU |
| | | | | UCAGAGGCAAAAACCUGCCCGUGCUGGAUCA |
| | | | | GCUGACCGAUCCCCCUGGCGUGAAAAGAGUG |
| | | | | UACCACAUCCAGCCCUCCCUGGAAGAUCCCUU |
| | | | | CCAGCCCCCUUCCAUCCCCAUCACCGUGUACU |
| | | | | ACGCCGUGCUGGAAAGAGCUUGCAGAUCCGU |
| | | | | GCUGCUGCACGCCCCCUCCGAAGCCCCUCAGA |
| | | | | UCGUGAGAGGCGCCUCCGAUGAAGCCAGAAA |
| | | | | ACACACCUACAACCUGACCAUCGCCUGGUAC |
| | | | | AGAAUGGGCGAUAACUGCGCCAUCCCCAUCA |
| | | | | CCGUGAUGGAAUACACCGAAUGCCCCUACAA |
| | | | | CAAAUCCCUGGGCGUGUGCCCCAUCAGAACC |
| | | | | CAGCCCAGAUGGUCCUACUACGAUUCCUUCU |
| | | | | CCGCCGUGUCCGAAGAUAACCUGGGCUUCCU |
| | | | | GAUGCACGCCCCCGCCUUCGAAACCGCCGGCA |
| | | | | CCUACCUGAGACUGGUGAAAAUCAACGAUUG |
| | | | | GACCGAAAUCACCCAGUUCAUCCUGGAACAC |
| | | | | AGAGCCAGAGCCUCCUGCAAAUACGCCCUGC |
| | | | | CCCUGAGAAUCCCUCCCGCCGCCUGCCUGACC |
| | | | | UCCAAAGCCUACCAGCAGGGCGUGACCGUGG |
| | | | | AUUCCAUCGGCAUGCUGCCCAGAUUCAUCCC |
| | | | | CGAAAACCAGAGAACCGUGGCCCUGUACUCC |
| | | | | CUGAAAAUCGCCGGCUGGCACGGCCCCAAAC |
| | | | | CCCCUUACACCUCCACCCUGCUGCCCCCUGAA |
| | | | | CUGUCCGAUACCACCAACGCCACCCAGCCCGA |
| | | | | ACUGGUGCCCGAAGAUCCCGAAGAUUCCGCC |
| | | | | CUGCUGGAAGAUCCCGCCGGCACCGUGUCCU |
| | | | | CCCAGAUCCCUCCCAACUGGCACAUCCCCUCC |
| | | | | AUCCAGGAUGUGGCCCCUCACCACUGA |
| 121 | IL2 | HSV-2 | | AUGCGCAUGCAGCUGCUGCUGCUGAUCGCCC |
| | | gE | | UGUCCCUGGCCCUGGUGACCAACUCCCGCACC |
| | | | | UCCUGGAAGCGCGUGACCUCCGGCGAGGACG |
| | | | | UGGUGCUGCUGCCCGCCCCCGCCGGCCCCGAG |
| | | | | GAGCGCACCCGCGCCCACAAGCUGCUGUGGG |
| | | | | CCGCCGAGCCCCUGGACGCCUGCGGCCCCCUG |
| | | | | CGCCCCUCCUGGGUGGCCCUGUGGCCCCCCCG |
| | | | | CCGCGUGCUGGAGACCGUGGUGGACGCCGCC |
| | | | | UGCAUGCGCGCCCCCGAGCCCCUGGCCAUCGC |
| | | | | CUACUCCCCCCCCUUCCCCGCCGGCGACGAGG |
| | | | | GCCUGUACUCCGAGCUGGCCUGGCGCGACCG |
| | | | | CGUGGCCGUGGUGAACGAGUCCCUGGUGAUC |
| | | | | UACGGCGCCCUGGAGACCGACUCCGGCCUGU |
| | | | | ACACCCUGUCCGUGGUGGGCCUGUCCGACGA |
| | | | | GGCCCGCCAGGUGGCCUCCGUGGUGCUGGUG |
| | | | | GUGGAGCCCGCCCCCGUGCCCACCCCCACCCC |
| | | | | CGACGACUACGACGAGGAGGACGACGCCGGC |
| | | | | GUGUCCGAGCGCACCCCCGUGUCCGUGCCCCC |
| | | | | CCCCACCCCCCCCCGCCGCCCCCCCGUGGCCC |
| | | | | CCCCCACCCACCCCCGCGUGAUCCCCGAGGUG |
| | | | | UCCCACGUGCGCGGCGUGACCGUGCACAUGG |
| | | | | AGACCCCCGAGGCCAUCCUGUUCGCCCCCGGC |
| | | | | GAGACCUUCGGCACCAACGUGUCCAUCCACG |
| | | | | CCAUCGCCCACGACGACGGCCCCUACGCCAUG |
| | | | | GACGUGGUGUGGAUGCGCUUCGACGUGCCCU |
| | | | | CCUCCUGCGCCGAGAUGCGCAUCUACGAGGC |
| | | | | CUGCCUGUACCACCCCCAGCUGCCCGAGUGCC |
| | | | | UGUCCCCCGCCGACGCCCCCUGCGCCGUGUCC |
| | | | | UCCUGGGCCUACCGCCUGGCCGUGCGCUCCUA |
| | | | | CGCCGGCUGCUCCCGCACCACCCCCCCCCCCC |
| | | | | GCUGCUUCGCCGAGGCCCGCAUGGAGCCCGU |
| | | | | GCCCGGCCUGGCCUGGCUGGCCUCCACCGUGA |
| | | | | ACCUGGAGUUCCAGCACGCCUCCCCCCAGCAC |
| | | | | GCCGGCCUGUACCUGUGCGUGGUGUACGUGG |
| | | | | ACGACCACAUCCACGCCUGGGGCCACAUGACC |
| | | | | AUCUCCACCGCCGCCCAGUACCGCAACGCCGU |
| | | | | GGUGGAGCAGCACCUGCCCCAGCGCCAGCCCG |
| | | | | AGCCCGUGGAGCCCACCCGCCCCCACGUGCGC |
| | | | | GCCUAA |
| 122 | IL2 | HSV-2 | Version | AUGAGAAUGCAGCUGCUGCUCCUGAUCGCCC |
| | | gE | 1 | UGUCUCUGGCCCUGGUCACCAAUAGCAGAAC |
| | | | | CAGCUGGAAAAGAGUGACCAGCGGCGAGGAU |
| | | | | GUGGUGCUGCUUCCUGCUCCUGCUGGCCCCG |
| | | | | AGGAAAGAACAAGAGCCCACAAACUGCUGUG |
| | | | | GGCCGCUGAGCCUCUUGAUGCCUGUGGACCU |
| | | | | CUCAGACCUAGCUGGGUUGCACUGUGGCCAC |
| | | | | CUCGGAGAGUGCUGGAAACAGUGGUGGAUGC |
| | | | | CGCCUGCAUGAGAGCCCCUGAACCUCUGGCC |
| | | | | AUUGCCUACUCUCCACCAUUUCCAGCCGGCG |
| | | | | ACGAGGGCCUGUAUUCUGAGCUUGCUUGGAG |
| | | | | AGACAGAGUGGCCGUGGUCAACGAGAGCCUG |
| | | | | GUUAUCUAUGGCGCCCUGGAAACCGACAGCG |
| | | | | GCCUGUACACACUGUCUGUCGUGGGCCUGUC |
| | | | | UGACGAGGCUAGACAGGUGGCAUCUGUGGUC |
| | | | | CUGGUGGUGGAACCUGCUCCAGUGCCUACAC |
| | | | | CUACACCUGACGACUACGACGAGGAAGAUGA |
| | | | | CGCUGGCGUCAGCGAGAGAACCCCUGUUUCU |
| | | | | GUGCCUCCUCCUACGCCUCCUCGUAGACCUCC |
| | | | | UGUUGCUCCUCCAACACACCCCAGAGUGAUC |
| | | | | CCUGAAGUGUCUCACGUGCGGGGCGUGACCG |
| | | | | UGCACAUGGAAACACCUGAGGCCAUCCUGUU |
| | | | | CGCCCCUGGCGAGACAUUUGGCACCAACGUG |
| | | | | UCCAUCCACGCUAUCGCCCACGACGAUGGCCC |
| | | | | UUACGCCAUGGAUGUCGUGUGGAUGAGAUUC |
| | | | | GACGUGCCCAGCAGCUGUGCCGAGAUGAGAA |
| | | | | UCUAUGAGGCCUGCCUGUAUCACCCUCAGCU |
| | | | | GCCCGAAUGUCUGAGCCCUGCUGAUGCCCCU |
| | | | | UGUGCCGUUAGCAGCUGGGCCUAUAGACUGG |
| | | | | CCGUGCGGUCUUAUGCCGGCUGCUCUAGAAC |
| | | | | AACCCCUCCUCCUCGGUGUUUCGCCGAGGCCA |
| | | | | GAAUGGAACCUGUUCCUGGACUGGCCUGGCU |
| | | | | GGCCUCCACAGUGAACCUGGAAUUUCAGCAC |
| | | | | GCCUCUCCACAGCACGCCGGCCUGUAUCUGU |
| | | | | GUGUGGUGUACGUGGACGAUCACAUCCACGC |
| | | | | CUGGGGCCACAUGACCAUCUCUACAGCCGCU |
| | | | | CAGUACCGGAACGCCGUGGUUGAACAGCAUC |
| | | | | UGCCUCAGAGACAGCCCGAGCCUGUGGAACC |
| | | | | UACAAGACCUCAUGUUCGGGCCUGA |
| 123 | IL2 | HSV-2 | Version | AUGCGAAUGCAGCUUCUGCUGCUCAUUGCCU |
| | | gE | 2 | UGUCCCUUGCCUUGGUGACCAACUCACGCAC |
| | | | | CUCUUGGAAACGCGUUACUUCCGGGGAGGAC |
| | | | | GUUGUCCUCCUUCCAGCACCCGCAGGACCUG |
| | | | | AAGAGAGGACUAGGGCCCACAAGCUGCUGUG |
| | | | | GGCCGCUGAACCUCUGGAUGCCUGUGGUCCU |
| | | | | CUGAGACCUAGCUGGGUCGCCCUUUGGCCAC |
| | | | | CUAGACGCGUUCUGGAGACGGUCGUGGAUGC |
| | | | | CGCGUGCAUGCGUGCACCCGAACCUCUGGCC |
| | | | | AUCGCCUAUAGUCCCCCUUUUCCCGCUGGCG |
| | | | | ACGAGGGGCUUUACUCCGAACUGGCCUGGCG |
| | | | | GGAUAGGGUGGCGGUGGUGAACGAGAGCCUC |
| | | | | GUCAUCUACGGUGCUCUGGAAACCGACUCAG |
| | | | | GACUGUAUACGCUCAGCGUUGUUGGCCUCUC |
| | | | | CGAUGAGGCUCGACAGGUUGCCUCCGUAGUG |
| | | | | CUGGUCGUAGAACCAGCCCCCGUACCAACACC |
| | | | | CACACCCGACGACUACGACGAAGAGGACGAC |
| | | | | GCUGGAGUUAGCGAAAGAACACCGGUGAGUG |
| | | | | UGCCACCUCCCACACCGCCAAGGAGACCCCCA |
| | | | | GUAGCACCUCCAACCCAUCCGAGAGUGAUUC |
| | | | | CCGAGGUCAGCCAUGUGCGCGGCGUAACUGU |
| | | | | GCACAUGGAGACGCCCGAAGCGAUACUGUUU |
| | | | | GCCCCUGGAGAGACAUUCGGCACCAAUGUGU |
| | | | | CCAUACACGCAAUUGCGCACGAUGAUGGCCC |
| | | | | AUACGCUAUGGACGUCGUCUGGAUGAGGUUC |
| | | | | GAUGUGCCUUCUUCUUGCGCCGAGAUGAGGA |
| | | | | UCUACGAGGCAUGCCUGUAUCACCCCCAAUU |
| | | | | GCCGGAGUGUCUGUCUCCCGCAGAUGCACCG |
| | | | | UGUGCAGUGAGUAGCUGGGCUUAUCGGUUGG |
| | | | | CUGUCCGGAGUUAUGCUGGGUGUUCACGGAC |
| | | | | CACCCCACCUCCACGUUGCUUUGCUGAAGCCA |
| | | | | GAAUGGAACCCGUGCCUGGUCUGGCUUGGCU |
| | | | | GGCAUCAACUGUCAACCUGGAGUUCCAGCAU |
| | | | | GCCUCUCCACAGCACGCAGGCCUGUAUCUCU |
| | | | | GCGUGGUGUACGUUGACGAUCACAUCCAUGC |
| | | | | GUGGGGGCAUAUGACCAUCAGCACAGCUGCC |
| | | | | CAGUACCGCAAUGCCGUCGUGGAGCAGCACC |
| | | | | UCCCCCAACGGCAGCCAGAACCAGUGGAGCCC |
| | | | | ACUCGGCCUCAUGUGCGAGCCUGA |
| 124 | IL2 | HSV-2 | Version | AUGAGAAUGCAGCUGCUGCUGCUGAUCGCCC |
| | | gE | 3 | UGUCCCUGGCCCUGGUGACCAACUCCAGAAC |
| | | | | CUCCUGGAAAAGAGUGACCUCCGGCGAAGAU |
| | | | | GUGGUGCUGCUGCCCGCCCCCGCCGGCCCCGA |
| | | | | AGAAAGAACCAGAGCCCACAAACUGCUGUGG |
| | | | | GCCGCCGAACCCCUGGAUGCCUGCGGCCCCCU |
| | | | | CAGACCCUCCUGGGUGGCCCUGUGGCCCCCUA |
| | | | | GAAGGGUGCUGGAAACCGUGGUGGAUGCCGC |
| | | | | CUGCAUGAGAGCCCCCGAACCCCUGGCCAUCG |
| | | | | CCUACUCCCCUCCCUUCCCCGCCGGCGAUGAA |
| | | | | GGCCUGUACUCCGAACUGGCCUGGAGAGAUA |
| | | | | GAGUGGCCGUGGUGAACGAAUCCCUGGUGAU |
| | | | | CUACGGCGCCCUGGAAACCGAUUCCGGCCUG |
| | | | | UACACCCUGUCCGUGGUGGGCCUGUCCGAUG |
| | | | | AAGCCAGACAGGUGGCCUCCGUGGUGCUGGU |
| | | | | GGUGGAACCCGCCCCCGUGCCCACCCCCACCC |
| | | | | CCGAUGAUUACGAUGAAGAAGAUGAUGCCGG |
| | | | | CGUGUCCGAAAGAACCCCCGUGUCCGUGCCCC |
| | | | | CUCCCACCCCUCCCCGCAGACCCCCUGUGGCC |
| | | | | CCUCCCACCCACCCCAGAGUGAUCCCCGAAGU |
| | | | | GUCCCACGUGAGAGGCGUGACCGUGCACAUG |
| | | | | GAAACCCCCGAAGCCAUCCUGUUCGCCCCCGG |
| | | | | CGAAACCUUCGGCACCAACGUGUCCAUCCAC |
| | | | | GCCAUCGCCCACGAUGAUGGCCCCUACGCCAU |
| | | | | GGAUGUGGUGUGGAUGAGAUUCGAUGUGCCC |
| | | | | UCCUCCUGCGCCGAAAUGAGAAUCUACGAAG |
| | | | | CCUGCCUGUACCACCCCCAGCUGCCCGAAUGC |
| | | | | CUGUCCCCCGCCGAUGCCCCCUGCGCCGUGUC |
| | | | | CUCCUGGGCCUACAGACUGGCCGUGAGAUCC |
| | | | | UACGCCGGCUGCUCCAGAACCACCCCUCCCCC |
| | | | | UAGAUGCUUCGCCGAAGCCAGAAUGGAACCC |
| | | | | GUGCCCGGCCUGGCCUGGCUGGCCUCCACCGU |
| | | | | GAACCUGGAAUUCCAGCACGCCAGCCCCCAGC |
| | | | | ACGCCGGCCUGUACCUGUGCGUGGUGUACGU |
| | | | | GGAUGAUCACAUCCACGCCUGGGGCCACAUG |
| | | | | ACCAUCUCCACCGCCGCCCAGUACAGAAACGC |
| | | | | CGUGGUGGAACAGCACCUGCCCCAGAGACAG |
| | | | | CCCGAACCCGUGGAACCCACCAGACCCCACGU |
| | | | | GAGAGCCUGA |
| 125 | HSV-2 | HSV-2 | | AUGGGCCGCCUGACCUCCGGCGUGGGCACCG |
| | gD- | gE | | CCGCCCUGCUGGUGGUGGCCGUGGGCCUGCG |
| | KYA | | | CGUGGUGUGCGCCAAGUACGCCCGCACCUCC |
| | | | | UGGAAGCGCGUGACCUCCGGCGAGGACGUGG |
| | | | | UGCUGCUGCCCGCCCCCGCCGGCCCCGAGGAG |
| | | | | CGCACCCGCGCCCACAAGCUGCUGUGGGCCGC |
| | | | | CGAGCCCCUGGACGCCUGCGGCCCCCUGCGCC |
| | | | | CCUCCUGGGUGGCCCUGUGGCCCCCCCGCCGC |
| | | | | GUGCUGGAGACCGUGGUGGACGCCGCCUGCA |
| | | | | UGCGCGCCCCCGAGCCCCUGGCCAUCGCCUAC |
| | | | | UCCCCCCCCUUCCCCGCCGGCGACGAGGGCCU |
| | | | | GUACUCCGAGCUGGCCUGGCGCGACCGCGUG |
| | | | | GCCGUGGUGAACGAGUCCCUGGUGAUCUACG |
| | | | | GCGCCCUGGAGACCGACUCCGGCCUGUACACC |
| | | | | CUGUCCGUGGUGGGCCUGUCCGACGAGGCCC |
| | | | | GCCAGGUGGCCUCCGUGGUGCUGGUGGUGGA |
| | | | | GCCCGCCCCCGUGCCCACCCCCACCCCCGACG |
| | | | | ACUACGACGAGGAGGACGACGCCGGCGUGUC |
| | | | | CGAGCGCACCCCCGUGUCCGUGCCCCCCCCCA |
| | | | | CCCCCCCCCGCCGCCCCCCCGUGGCCCCCCCC |
| | | | | ACCCACCCCCGCGUGAUCCCCGAGGUGUCCCA |
| | | | | CGUGCGCGGCGUGACCGUGCACAUGGAGACC |
| | | | | CCCGAGGCCAUCCUGUUCGCCCCCGGCGAGAC |
| | | | | CUUCGGCACCAACGUGUCCAUCCACGCCAUCG |
| | | | | CCCACGACGACGGCCCCUACGCCAUGGACGUG |
| | | | | GUGUGGAUGCGCUUCGACGUGCCCUCCUCCU |
| | | | | GCGCCGAGAUGCGCAUCUACGAGGCCUGCCU |
| | | | | GUACCACCCCCAGCUGCCCGAGUGCCUGUCCC |
| | | | | CCGCCGACGCCCCCUGCGCCGUGUCCUCCUGG |
| | | | | GCCUACCGCCUGGCCGUGCGCUCCUACGCCGG |
| | | | | CUGCUCCCGCACCACCCCCCCCCCCCGCUGCU |
| | | | | UCGCCGAGGCCCGCAUGGAGCCCGUGCCCGGC |
| | | | | CUGGCCUGGCUGGCCUCCACCGUGAACCUGG |
| | | | | AGUUCCAGCACGCCUCCCCCCAGCACGCCGGC |
| | | | | CUGUACCUGUGCGUGGUGUACGUGGACGACC |
| | | | | ACAUCCACGCCUGGGGCCACAUGACCAUCUCC |
| | | | | ACCGCCGCCCAGUACCGCAACGCCGUGGUGG |
| | | | | AGCAGCACCUGCCCCAGCGCCAGCCCGAGCCC |
| | | | | GUGGAGCCCACCCGCCCCCACGUGCGCGCCUA |
| | | | | A |
| 126 | HSV-2 | HSV-2 | Version | AUGGGCAGACUGACAUCUGGCGUGGGAACAG |
| | gD - | gE | 1 | CUGCUCUGCUGGUGGUUGCUGUGGGCCUGAG |
| | KYA | | | AGUCGUGUGUGCCAAAUACGCCAGAACCAGC |
| | | | | UGGAAAAGAGUGACCAGCGGCGAGGAUGUGG |
| | | | | UGCUGCUUCCUGCUCCUGCUGGCCCCGAGGA |
| | | | | AAGAACAAGAGCCCACAAACUGCUGUGGGCC |
| | | | | GCUGAGCCUCUUGAUGCCUGUGGACCUCUCA |
| | | | | GACCUAGCUGGGUUGCACUGUGGCCACCUCG |
| | | | | GAGAGUGCUGGAAACAGUGGUGGAUGCCGCC |
| | | | | UGCAUGAGAGCCCCUGAACCUCUGGCCAUUG |
| | | | | CCUACUCUCCACCAUUUCCAGCCGGCGACGAG |
| | | | | GGCCUGUAUUCUGAGCUUGCUUGGAGAGACA |
| | | | | GAGUGGCCGUGGUCAACGAGAGCCUGGUUAU |
| | | | | CUAUGGCGCCCUGGAAACCGACAGCGGCCUG |
| | | | | UACACACUGUCUGUCGUGGGCCUGUCUGACG |
| | | | | AGGCUAGACAGGUGGCAUCUGUGGUCCUGGU |
| | | | | GGUGGAACCUGCUCCAGUGCCUACACCUACA |
| | | | | CCUGACGACUACGACGAGGAAGAUGACGCUG |
| | | | | GCGUCAGCGAGAGAACCCCUGUUUCUGUGCC |
| | | | | UCCUCCUACGCCUCCUCGUAGACCUCCUGUUG |
| | | | | CUCCUCCAACACACCCCAGAGUGAUCCCUGAA |
| | | | | GUGUCUCACGUGCGGGGCGUGACCGUGCACA |
| | | | | UGGAAACACCUGAGGCCAUCCUGUUCGCCCC |
| | | | | UGGCGAGACAUUUGGCACCAACGUGUCCAUC |
| | | | | CACGCUAUCGCCCACGACGAUGGCCCUUACGC |
| | | | | CAUGGAUGUCGUGUGGAUGAGAUUCGACGUG |
| | | | | CCCAGCAGCUGUGCCGAGAUGAGAAUCUAUG |
| | | | | AGGCCUGCCUGUAUCACCCUCAGCUGCCCGA |
| | | | | AUGUCUGAGCCCUGCUGAUGCCCCUUGUGCC |
| | | | | GUUAGCAGCUGGGCCUAUAGACUGGCCGUGC |
| | | | | GGUCUUAUGCCGGCUGCUCUAGAACAACCCC |
| | | | | UCCUCCUCGGUGUUUCGCCGAGGCCAGAAUG |
| | | | | GAACCUGUUCCUGGACUGGCCUGGCUGGCCU |
| | | | | CCACAGUGAACCUGGAAUUUCAGCACGCCUC |
| | | | | UCCACAGCACGCCGGCCUGUAUCUGUGUGUG |
| | | | | GUGUACGUGGACGAUCACAUCCACGCCUGGG |
| | | | | GCCACAUGACCAUCUCUACAGCCGCUCAGUA |
| | | | | CCGGAACGCCGUGGUUGAACAGCAUCUGCCU |
| | | | | CAGAGACAGCCCGAGCCUGUGGAACCUACAA |
| | | | | GACCUCAUGUUCGGGCCUGA |
| 127 | HSV-2 | HSV-2 | Version | AUGGGGAGACUCACAUCAGGCGUAGGAACCG |
| | gD- | gE | 2 | CUGCCCUGUUGGUCGUGGCCGUUGGUCUGAG |
| | KYA | | | AGUUGUGUGUGCCAAAUAUGCUCGCACCUCU |
| | | | | UGGAAACGCGUUACUUCCGGGGAGGACGUUG |
| | | | | UCCUCCUUCCAGCACCCGCAGGACCUGAGGA |
| | | | | AAGGACUAGGGCCCACAAGCUGCUGUGGGCC |
| | | | | GCUGAACCUCUGGAUGCCUGUGGUCCUCUGA |
| | | | | GACCUAGCUGGGUCGCCCUUUGGCCACCUAG |
| | | | | ACGCGUUCUGGAGACGGUCGUGGAUGCCGCG |
| | | | | UGCAUGCGUGCACCCGAACCUCUGGCCAUCG |
| | | | | CCUAUAGUCCCCCUUUUCCCGCUGGCGACGA |
| | | | | GGGGCUUUACUCCGAACUGGCCUGGCGGGAU |
| | | | | AGGGUGGCGGUGGUGAACGAGAGCCUCGUCA |
| | | | | UCUACGGUGCUCUGGAAACCGACUCAGGACU |
| | | | | GUAUACGCUCAGCGUUGUUGGCCUCUCCGAU |
| | | | | GAGGCUCGACAGGUUGCCUCCGUAGUGCUGG |
| | | | | UCGUAGAACCAGCCCCCGUACCAACACCCACA |
| | | | | CCCGACGACUACGACGAGGAGGACGACGCUG |
| | | | | GAGUUAGCGAAAGAACACCGGUGAGUGUGCC |
| | | | | ACCUCCCACACCGCCAAGGAGACCCCCAGUAG |
| | | | | CACCUCCAACCCAUCCGAGAGUGAUUCCCGA |
| | | | | GGUCAGCCAUGUGCGCGGCGUAACUGUGCAC |
| | | | | AUGGAGACGCCCGAAGCGAUACUGUUUGCCC |
| | | | | CUGGAGAGACAUUCGGCACCAAUGUGUCCAU |
| | | | | ACACGCAAUUGCGCACGAUGAUGGCCCAUAC |
| | | | | GCUAUGGACGUCGUCUGGAUGAGGUUCGAUG |
| | | | | UGCCUUCUUCUUGCGCCGAGAUGAGGAUCUA |
| | | | | CGAGGCAUGCCUGUAUCACCCCCAAUUGCCG |
| | | | | GAGUGUCUGUCUCCCGCAGAUGCACCGUGUG |
| | | | | CAGUGAGUAGCUGGGCUUAUCGGUUGGCUGU |
| | | | | CCGGAGUUAUGCUGGGUGUUCACGGACCACC |
| | | | | CCACCUCCACGUUGCUUUGCUGAAGCCAGAA |
| | | | | UGGAACCCGUGCCUGGUCUGGCUUGGCUGGC |
| | | | | AUCAACUGUCAACCUGGAGUUCCAGCAUGCC |
| | | | | UCUCCACAGCACGCAGGCCUGUAUCUCUGCG |
| | | | | UGGUGUACGUUGACGAUCACAUCCAUGCGUG |
| | | | | GGGGCAUAUGACCAUCAGCACAGCUGCCCAG |
| | | | | UACCGCAAUGCCGUCGUGGAGCAGCACCUCC |
| | | | | CCCAACGGCAGCCAGAACCAGUGGAGCCCAC |
| | | | | UCGGCCUCAUGUGCGAGCCUGA |
| 128 | HSV-2 | HSV-2 | Version | AUGGGGAGACUCACAUCAGGCGUAGGAACCG |
| | gD- | gE | 2.1 | CUGCCCUGUUGGUCGUGGCCGUUGGUCUGAG |
| | KYA | | | AGUUGUGUGUGCCAAAUAUGCUCGCACCUCU |
| | | | | UGGAAACGCGUUACUUCCGGGGAGGACGUUG |
| | | | | UCCUCCUUCCAGCACCCGCAGGACCUGAAGA |
| | | | | GAGGACUAGGGCCCACAAGCUGCUGUGGGCC |
| | | | | GCUGAACCUCUGGAUGCCUGUGGUCCUCUGA |
| | | | | GACCUAGCUGGGUCGCCCUUUGGCCACCUAG |
| | | | | ACGCGUUCUGGAGACGGUCGUGGAUGCCGCG |
| | | | | UGCAUGCGUGCACCCGAACCUCUGGCCAUCG |
| | | | | CCUAUAGUCCCCCUUUUCCCGCUGGCGACGA |
| | | | | GGGGCUUUACUCCGAACUGGCCUGGCGGGAU |
| | | | | AGGGUGGCGGUGGUGAACGAGAGCCUCGUCA |
| | | | | UCUACGGUGCUCUGGAAACCGACUCAGGACU |
| | | | | GUAUACGCUCAGCGUUGUUGGCCUCUCCGAU |
| | | | | GAGGCUCGACAGGUUGCCUCCGUAGUGCUGG |
| | | | | UCGUAGAACCAGCCCCCGUACCAACACCCACA |
| | | | | CCCGACGACUACGACGAAGAGGACGACGCUG |
| | | | | GAGUUAGCGAAAGAACACCGGUGAGUGUGCC |
| | | | | ACCUCCCACACCGCCAAGGAGACCCCCAGUAG |
| | | | | CACCUCCAACCCAUCCGAGAGUGAUUCCCGA |
| | | | | GGUCAGCCAUGUGCGCGGCGUAACUGUGCAC |
| | | | | AUGGAGACGCCCGAAGCGAUACUGUUUGCCC |
| | | | | CUGGAGAGACAUUCGGCACCAAUGUGUCCAU |
| | | | | ACACGCAAUUGCGCACGAUGAUGGCCCAUAC |
| | | | | GCUAUGGACGUCGUCUGGAUGAGGUUCGAUG |
| | | | | UGCCUUCUUCUUGCGCCGAGAUGAGGAUCUA |
| | | | | CGAGGCAUGCCUGUAUCACCCCCAAUUGCCG |
| | | | | GAGUGUCUGUCUCCCGCAGAUGCACCGUGUG |
| | | | | CAGUGAGUAGCUGGGCUUAUCGGUUGGCUGU |
| | | | | CCGGAGUUAUGCUGGGUGUUCACGGACCACC |
| | | | | CCACCUCCACGUUGCUUUGCUGAAGCCAGAA |
| | | | | UGGAACCCGUGCCUGGUCUGGCUUGGCUGGC |
| | | | | AUCAACUGUCAACCUGGAGUUCCAGCAUGCC |
| | | | | UCUCCACAGCACGCAGGCCUGUAUCUCUGCG |
| | | | | UGGUGUACGUUGACGAUCACAUCCAUGCGUG |
| | | | | GGGGCAUAUGACCAUCAGCACAGCUGCCCAG |
| | | | | UACCGCAAUGCCGUCGUGGAGCAGCACCUCC |
| | | | | CCCAACGGCAGCCAGAACCAGUGGAGCCCAC |
| | | | | UCGGCCUCAUGUGCGAGCCUGA |
| 353 | HSV-2 | HSV-2 | Version | AUGGGGAGACUCACAUCAGGCGUAGGAACCG |
| | gD- | gE | 2.2 | CUGCCCUGUUGGUCGUGGCCGUUGGUCUGAG |
| | KYA | | | AGUUGUGUGUGCCAAAUAUGCUCGCACCUCU |
| | | | | UGGAAACGCGUUACUUCCGGGGAGGACGUUG |
| | | | | UCCUCCUUCCAGCACCCGCAGGACCUGAGGA |
| | | | | AAGGACUAGGGCCCACAAGCUGCUGUGGGCC |
| | | | | GCUGAACCUCUGGAUGCCUGUGGUCCUCUGA |
| | | | | GACCUAGCUGGGUCGCCCUUUGGCCACCUAG |
| | | | | ACGCGUUCUGGAGACGGUCGUGGAUGCCGCG |
| | | | | UGCAUGCGUGCACCCGAACCUCUGGCCAUCG |
| | | | | CCUAUAGUCCCCCUUUUCCCGCUGGCGACGA |
| | | | | GGGGCUUUACUCCGAACUGGCCUGGCGGGAU |
| | | | | AGGGUGGCGGUGGUGAACGAGAGCCUCGUCA |
| | | | | UCUACGGUGCUCUGGAAACCGACUCAGGACU |
| | | | | GUAUACGCUCAGCGUUGUUGGCCUCUCCGAU |
| | | | | GAGGCUCGACAGGUUGCCUCCGUAGUGCUGG |
| | | | | UCGUAGAACCAGCCCCCGUACCAACACCCACA |
| | | | | CCCGACGACUACGACGAGGAGGACGACGCUG |
| | | | | GAGUUAGCGAAAGAACACCGGUGAGUGUGCC |
| | | | | ACCUCCCACACCGCCAAGGAGACCCCCAGUAG |
| | | | | CACCUCCAACCCAUCCGAGAGUGAUUCCCGA |
| | | | | GGUCAGCCAUGUGCGCGGCGUAACUGUGCAC |
| | | | | AUGGAGACGCCCGAAGCGAUACUGUUUGCCC |
| | | | | CUGGAGAGACAUUCGGCACCAAUGUGUCCAU |
| | | | | ACACGCAAUUGCGCACGAUGAUGGCCCAUAC |
| | | | | GCUAUGGACGUCGUCUGGAUGAGGUUCGAUG |
| | | | | UGCCUUCUUCUUGCGCCGAGAUGAGGAUCUA |
| | | | | CGAGGCAUGCCUGUAUCACCCCCAAUUGCCG |
| | | | | GAGUGUCUGUCUCCCGCAGAUGCACCGUGUG |
| | | | | CAGUGAGUAGCUGGGCUUAUCGGUUGGCUGU |
| | | | | CCGGAGUUAUGCUGGGUGUUCACGGACCACC |
| | | | | CCACCUCCACGUUGCUUUGCUGAAGCCAGAA |
| | | | | UGGAACCCGUGCCUGGUCUGGCUUGGCUGGC |
| | | | | AUCAACUGUCAACCUGGAGUUCCAGCAUGCC |
| | | | | UCUCCACAGCACGCAGGCCUGUAUCUCUGCG |
| | | | | UGGUGUACGUUGACGAUCACAUCCAUGCGUG |
| | | | | GGGGCAUAUGACCAUCAGCACAGCUGCCCAG |
| | | | | UACCGCAAUGCCGUCGUGGAGCAGCACCUCC |
| | | | | CCCAACGGCAGCCAGAACCAGUGGAGCCCAC |
| | | | | UCGGCCUCAUGUGCGAGCCUGAUAA |
| 129 | HSV-2 | HSV-2 | Version | AUGGGCAGACUGACCUCCGGCGUGGGCACCG |
| | gD- | gE | 3 | CCGCCCUGCUGGUGGUGGCCGUGGGCCUGAG |
| | KYA | | | AGUGGUGUGCGCCAAAUACGCCAGAACCUCC |
| | | | | UGGAAAAGAGUGACCUCCGGCGAAGAUGUGG |
| | | | | UGCUGCUGCCCGCCCCCGCCGGCCCCGAAGAA |
| | | | | AGAACCAGAGCCCACAAACUGCUGUGGGCCG |
| | | | | CCGAACCCCUGGAUGCCUGCGGCCCCCUCAGA |
| | | | | CCCUCCUGGGUGGCCCUGUGGCCCCCUAGAA |
| | | | | GGGUGCUGGAAACCGUGGUGGAUGCCGCCUG |
| | | | | CAUGAGAGCCCCCGAACCCCUGGCCAUCGCCU |
| | | | | ACUCCCCUCCCUUCCCCGCCGGCGAUGAAGGC |
| | | | | CUGUACUCCGAACUGGCCUGGAGAGAUAGAG |
| | | | | UGGCCGUGGUGAACGAAUCCCUGGUGAUCUA |
| | | | | CGGCGCCCUGGAAACCGAUUCCGGCCUGUAC |
| | | | | ACCCUGUCCGUGGUGGGCCUGUCCGAUGAAG |
| | | | | CCAGACAGGUGGCCUCCGUGGUGCUGGUGGU |
| | | | | GGAACCCGCCCCCGUGCCCACCCCCACCCCCG |
| | | | | AUGAUUACGAUGAAGAAGAUGAUGCCGGCGU |
| | | | | GUCCGAAAGAACCCCCGUGUCCGUGCCCCCUC |
| | | | | CCACCCCUCCCCGCAGACCCCCUGUGGCCCCU |
| | | | | CCCACCCACCCCAGAGUGAUCCCCGAAGUGUC |
| | | | | CCACGUGAGAGGCGUGACCGUGCACAUGGAA |
| | | | | ACCCCCGAAGCCAUCCUGUUCGCCCCCGGCGA |
| | | | | AACCUUCGGCACCAACGUGUCCAUCCACGCCA |
| | | | | UCGCCCACGAUGAUGGCCCCUACGCCAUGGA |
| | | | | UGUGGUGUGGAUGAGAUUCGAUGUGCCCUCC |
| | | | | UCCUGCGCCGAAAUGAGAAUCUACGAAGCCU |
| | | | | GCCUGUACCACCCCCAGCUGCCCGAAUGCCUG |
| | | | | UCCCCCGCCGAUGCCCCCUGCGCCGUGUCCUC |
| | | | | CUGGGCCUACAGACUGGCCGUGAGAUCCUAC |
| | | | | GCCGGCUGCUCCAGAACCACCCCUCCCCCUAG |
| | | | | AUGCUUCGCCGAAGCCAGAAUGGAACCCGUG |
| | | | | CCCGGCCUGGCCUGGCUGGCCUCCACCGUGAA |
| | | | | CCUGGAAUUCCAGCACGCCAGCCCCCAGCACG |
| | | | | CCGGCCUGUACCUGUGCGUGGUGUACGUGGA |
| | | | | UGAUCACAUCCACGCCUGGGGCCACAUGACC |
| | | | | AUCUCCACCGCCGCCCAGUACAGAAACGCCGU |
| | | | | GGUGGAACAGCACCUGCCCCAGAGACAGCCC |
| | | | | GAACCCGUGGAACCCACCAGACCCCACGUGA |
| | | | | GAGCCUGA |
| 130 | HSV-2 | HSV-2 | Version | AUGGCACGGGGAGCCGGAUUGGUGUUCUUUG |
| | gE | gE | 2 | UGGGCGUGUGGGUGGUGAGCUGCUUGGCAGC |
| | | | | CGCACCACGCACCUCUUGGAAACGCGUUACU |
| | | | | UCCGGGGAGGACGUUGUCCUCCUUCCAGCAC |
| | | | | CCGCAGGACCUGAGGAAAGGACUAGGGCCCA |
| | | | | CAAGCUGCUGUGGGCCGCUGAACCUCUGGAU |
| | | | | GCCUGUGGUCCUCUGAGACCUAGCUGGGUCG |
| | | | | CCCUUUGGCCACCUAGACGCGUUCUGGAGAC |
| | | | | GGUCGUGGAUGCCGCGUGCAUGCGUGCACCC |
| | | | | GAACCUCUGGCCAUCGCCUAUAGUCCCCCUU |
| | | | | UUCCCGCUGGCGACGAGGGGCUUUACUCCGA |
| | | | | ACUGGCCUGGCGGGAUAGGGUGGCGGUGGUG |
| | | | | AACGAGAGCCUCGUCAUCUACGGUGCUCUGG |
| | | | | AAACCGACUCAGGACUGUAUACGCUCAGCGU |
| | | | | UGUUGGCCUCUCCGAUGAGGCUCGACAGGUU |
| | | | | GCCUCCGUAGUGCUGGUCGUAGAACCAGCCC |
| | | | | CCGUACCAACACCCACACCCGACGACUACGAC |
| | | | | GAGGAGGACGACGCUGGAGUUAGCGAAAGAA |
| | | | | CACCGGUGAGUGUGCCACCUCCCACACCGCCA |
| | | | | AGGAGACCCCCAGUAGCACCUCCAACCCAUCC |
| | | | | GAGAGUGAUUCCCGAGGUCAGCCAUGUGCGC |
| | | | | GGCGUAACUGUGCACAUGGAGACGCCCGAAG |
| | | | | CGAUACUGUUUGCCCCUGGAGAGACAUUCGG |
| | | | | CACCAAUGUGUCCAUACACGCAAUUGCGCAC |
| | | | | GAUGAUGGCCCAUACGCUAUGGACGUCGUCU |
| | | | | GGAUGAGGUUCGAUGUGCCUUCUUCUUGCGC |
| | | | | CGAGAUGAGGAUCUACGAGGCAUGCCUGUAU |
| | | | | CACCCCCAAUUGCCGGAGUGUCUGUCUCCCGC |
| | | | | AGAUGCACCGUGUGCAGUGAGUAGCUGGGCU |
| | | | | UAUCGGUUGGCUGUCCGGAGUUAUGCUGGGU |
| | | | | GUUCACGGACCACCCCACCUCCACGUUGCUUU |
| | | | | GCUGAAGCCAGAAUGGAACCCGUGCCUGGUC |
| | | | | UGGCUUGGCUGGCAUCAACUGUCAACCUGGA |
| | | | | GUUCCAGCAUGCCUCUCCACAGCACGCAGGCC |
| | | | | UGUAUCUCUGCGUGGUGUACGUUGACGAUCA |
| | | | | CAUCCAUGCGUGGGGGCAUAUGACCAUCAGC |
| | | | | ACAGCUGCCCAGUACCGCAAUGCCGUCGUGG |
| | | | | AGCAGCACCUCCCCCAACGGCAGCCAGAACCA |
| | | | | GUGGAGCCCACUCGGCCUCAUGUGCGAGCCU |
| | | | | GA |
| 322 | HSV-2 | HSV-2 | Version | AUGGCACGGGGCGCAGGUUUGGUCUUUUUCG |
| | gE | gE | 2.1 | UGGGCGUGUGGGUGGUGAGCUGCUUGGCAGC |
| | | | | CGCACCACGCACCUCUUGGAAACGCGUUACU |
| | | | | UCCGGGGAGGACGUUGUCCUCCUUCCAGCAC |
| | | | | CCGCAGGACCUGAGGAAAGGACUAGGGCCCA |
| | | | | CAAGCUGCUGUGGGCCGCUGAACCUCUGGAU |
| | | | | GCCUGUGGUCCUCUGAGACCUAGCUGGGUCG |
| | | | | CCCUUUGGCCACCUAGACGCGUUCUGGAGAC |
| | | | | GGUCGUGGAUGCCGCGUGCAUGCGUGCACCC |
| | | | | GAACCUCUGGCCAUCGCCUAUAGUCCCCCUU |
| | | | | UUCCCGCUGGCGACGAGGGGCUUUACUCCGA |
| | | | | ACUGGCCUGGCGGGAUAGGGUGGCGGUGGUG |
| | | | | AACGAGAGCCUCGUCAUCUACGGUGCUCUGG |
| | | | | AAACCGACUCAGGACUGUAUACGCUCAGCGU |
| | | | | UGUUGGCCUCUCCGAUGAGGCUCGACAGGUU |
| | | | | GCCUCCGUAGUGCUGGUCGUAGAACCAGCCC |
| | | | | CCGUACCAACACCCACACCCGACGACUACGAC |
| | | | | GAGGAGGACGACGCUGGAGUUAGCGAAAGAA |
| | | | | CACCGGUGAGUGUGCCACCUCCCACACCGCCA |
| | | | | AGGAGACCCCCAGUAGCACCUCCAACCCAUCC |
| | | | | GAGAGUGAUUCCCGAGGUCAGCCAUGUGCGC |
| | | | | GGCGUAACUGUGCACAUGGAGACGCCCGAAG |
| | | | | CGAUACUGUUUGCCCCUGGAGAGACAUUCGG |
| | | | | CACCAAUGUGUCCAUACACGCAAUUGCGCAC |
| | | | | GAUGAUGGCCCAUACGCUAUGGACGUCGUCU |
| | | | | GGAUGAGGUUCGAUGUGCCUUCUUCUUGCGC |
| | | | | CGAGAUGAGGAUCUACGAGGCAUGCCUGUAU |
| | | | | CACCCCCAAUUGCCGGAGUGUCUGUCUCCCGC |
| | | | | AGAUGCACCGUGUGCAGUGAGUAGCUGGGCU |
| | | | | UAUCGGUUGGCUGUCCGGAGUUAUGCUGGGU |
| | | | | GUUCACGGACCACCCCACCUCCACGUUGCUUU |
| | | | | GCUGAAGCCAGAAUGGAACCCGUGCCUGGUC |
| | | | | UGGCUUGGCUGGCAUCAACUGUCAACCUGGA |
| | | | | GUUCCAGCAUGCCUCUCCACAGCACGCAGGCC |
| | | | | UGUAUCUCUGCGUGGUGUACGUUGACGAUCA |
| | | | | CAUCCAUGCGUGGGGGCAUAUGACCAUCAGC |
| | | | | ACAGCUGCCCAGUACCGCAAUGCCGUCGUGG |
| | | | | AGCAGCACCUCCCCCAACGGCAGCCAGAACCA |
| | | | | GUGGAGCCCACUCGGCCUCAUGUGCGAGCCU |
| | | | | GA |
| 323 | HSV-2 | HSV-2 | Version | AUGGCGAGAGGAGCCGGACUCGUGUUCUUUG |
| | gE | gE | 2.2 | UGGGAGUCUGGGUUGUGAGCUGCCUGGCAGC |
| | | | | AGCUCCACGCACCUCUUGGAAACGCGUUACU |
| | | | | UCCGGGGAGGACGUUGUCCUCCUUCCAGCAC |
| | | | | CCGCAGGACCUGAAGAGAGGACUAGGGCCCA |
| | | | | CAAGCUGCUGUGGGCCGCUGAACCUCUGGAU |
| | | | | GCCUGUGGUCCUCUGAGACCUAGCUGGGUCG |
| | | | | CCCUUUGGCCACCUAGACGCGUUCUGGAGAC |
| | | | | GGUCGUGGAUGCCGCGUGCAUGCGUGCACCC |
| | | | | GAACCUCUGGCCAUCGCCUAUAGUCCCCCUU |
| | | | | UUCCCGCUGGCGACGAGGGGCUUUACUCCGA |
| | | | | ACUGGCCUGGCGGGAUAGGGUGGCGGUGGUG |
| | | | | AACGAGAGCCUCGUCAUCUACGGUGCUCUGG |
| | | | | AAACCGACUCAGGACUGUAUACGCUCAGCGU |
| | | | | UGUUGGCCUCUCCGAUGAGGCUCGACAGGUU |
| | | | | GCCUCCGUAGUGCUGGUCGUAGAACCAGCCC |
| | | | | CCGUACCAACACCCACACCCGACGACUACGAC |
| | | | | GAAGAGGACGACGCUGGAGUUAGCGAAAGAA |
| | | | | CACCGGUGAGUGUGCCACCUCCCACACCGCCA |
| | | | | AGGAGACCCCCAGUAGCACCUCCAACCCAUCC |
| | | | | GAGAGUGAUUCCCGAGGUCAGCCAUGUGCGC |
| | | | | GGCGUAACUGUGCACAUGGAGACGCCCGAAG |
| | | | | CGAUACUGUUUGCCCCUGGAGAGACAUUCGG |
| | | | | CACCAAUGUGUCCAUACACGCAAUUGCGCAC |
| | | | | GAUGAUGGCCCAUACGCUAUGGACGUCGUCU |
| | | | | GGAUGAGGUUCGAUGUGCCUUCUUCUUGCGC |
| | | | | CGAGAUGAGGAUCUACGAGGCAUGCCUGUAU |
| | | | | CACCCCCAAUUGCCGGAGUGUCUGUCUCCCGC |
| | | | | AGAUGCACCGUGUGCAGUGAGUAGCUGGGCU |
| | | | | UAUCGGUUGGCUGUCCGGAGUUAUGCUGGGU |
| | | | | GUUCACGGACCACCCCACCUCCACGUUGCUUU |
| | | | | GCUGAAGCCAGAAUGGAACCCGUGCCUGGUC |
| | | | | UGGCUUGGCUGGCAUCAACUGUCAACCUGGA |
| | | | | GUUCCAGCAUGCCUCUCCACAGCACGCAGGCC |
| | | | | UGUAUCUCUGCGUGGUGUACGUUGACGAUCA |
| | | | | CAUCCAUGCGUGGGGGCAUAUGACCAUCAGC |
| | | | | ACAGCUGCCCAGUACCGCAAUGCCGUCGUGG |
| | | | | AGCAGCACCUCCCCCAACGGCAGCCAGAACCA |
| | | | | GUGGAGCCCACUCGGCCUCAUGUGCGAGCCU |
| | | | | GA |
| 324 | HSV-2 | HSV-2 | Version | AUGGCGAGAGGAGCCGGGCUCGUGUUCUUUG |
| | gE | gE | 2.3 | UGGGCGUAUGGGUCGUUUCCUGCCUGGCUGC |
| | | | | CGCACCCCGCACCUCUUGGAAACGCGUUACU |
| | | | | UCCGGGGAGGACGUUGUCCUCCUUCCAGCAC |
| | | | | CCGCAGGACCUGAGGAAAGGACUAGGGCCCA |
| | | | | CAAGCUGCUGUGGGCCGCUGAACCUCUGGAU |
| | | | | GCCUGUGGUCCUCUGAGACCUAGCUGGGUCG |
| | | | | CCCUUUGGCCACCUAGACGCGUUCUGGAGAC |
| | | | | GGUCGUGGAUGCCGCGUGCAUGCGUGCACCC |
| | | | | GAACCUCUGGCCAUCGCCUAUAGUCCCCCUU |
| | | | | UUCCCGCUGGCGACGAGGGGCUUUACUCCGA |
| | | | | ACUGGCCUGGCGGGAUAGGGUGGCGGUGGUG |
| | | | | AACGAGAGCCUCGUCAUCUACGGUGCUCUGG |
| | | | | AAACCGACUCAGGACUGUAUACGCUCAGCGU |
| | | | | UGUUGGCCUCUCCGAUGAGGCUCGACAGGUU |
| | | | | GCCUCCGUAGUGCUGGUCGUAGAACCAGCCC |
| | | | | CCGUACCAACACCCACACCCGACGACUACGAC |
| | | | | GAGGAGGACGACGCUGGAGUUAGCGAAAGAA |
| | | | | CACCGGUGAGUGUGCCACCUCCCACACCGCCA |
| | | | | AGGAGACCCCCAGUAGCACCUCCAACCCAUCC |
| | | | | GAGAGUGAUUCCCGAGGUCAGCCAUGUGCGC |
| | | | | GGCGUAACUGUGCACAUGGAGACGCCCGAAG |
| | | | | CGAUACUGUUUGCCCCUGGAGAGACAUUCGG |
| | | | | CACCAAUGUGUCCAUACACGCAAUUGCGCAC |
| | | | | GAUGAUGGCCCAUACGCUAUGGACGUCGUCU |
| | | | | GGAUGAGGUUCGAUGUGCCUUCUUCUUGCGC |
| | | | | CGAGAUGAGGAUCUACGAGGCAUGCCUGUAU |
| | | | | CACCCCCAAUUGCCGGAGUGUCUGUCUCCCGC |
| | | | | AGAUGCACCGUGUGCAGUGAGUAGCUGGGCU |
| | | | | UAUCGGUUGGCUGUCCGGAGUUAUGCUGGGU |
| | | | | GUUCACGGACCACCCCACCUCCACGUUGCUUU |
| | | | | GCUGAAGCCAGAAUGGAACCCGUGCCUGGUC |
| | | | | UGGCUUGGCUGGCAUCAACUGUCAACCUGGA |
| | | | | GUUCCAGCAUGCCUCUCCACAGCACGCAGGCC |
| | | | | UGUAUCUCUGCGUGGUGUACGUUGACGAUCA |
| | | | | CAUCCAUGCGUGGGGGCAUAUGACCAUCAGC |
| | | | | ACAGCUGCCCAGUACCGCAAUGCCGUCGUGG |
| | | | | AGCAGCACCUCCCCCAACGGCAGCCAGAACCA |
| | | | | GUGGAGCCCACUCGGCCUCAUGUGCGAGCCU |
| | | | | GA |
| 325 | HSV-2 | HSV-2 | Version | AUGGCGAGAGGAGCCGGACUCGUGUUCUUUG |
| | gE- | gE | 2 | UGGGAGUCUGGGUUGUGAGCUGCCUGGUAGU |
| | LVVV | | | AGUUCCACGCACCUCUUGGAAACGCGUUACU |
| | | | | UCCGGGGAGGACGUUGUCCUCCUUCCAGCAC |
| | | | | CCGCAGGACCUGAAGAGAGGACUAGGGCCCA |
| | | | | CAAGCUGCUGUGGGCCGCUGAACCUCUGGAU |
| | | | | GCCUGUGGUCCUCUGAGACCUAGCUGGGUCG |
| | | | | CCCUUUGGCCACCUAGACGCGUUCUGGAGAC |
| | | | | GGUCGUGGAUGCCGCGUGCAUGCGUGCACCC |
| | | | | GAACCUCUGGCCAUCGCCUAUAGUCCCCCUU |
| | | | | UUCCCGCUGGCGACGAGGGGCUUUACUCCGA |
| | | | | ACUGGCCUGGCGGGAUAGGGUGGCGGUGGUG |
| | | | | AACGAGAGCCUCGUCAUCUACGGUGCUCUGG |
| | | | | AAACCGACUCAGGACUGUAUACGCUCAGCGU |
| | | | | UGUUGGCCUCUCCGAUGAGGCUCGACAGGUU |
| | | | | GCCUCCGUAGUGCUGGUCGUAGAACCAGCCC |
| | | | | CCGUACCAACACCCACACCCGACGACUACGAC |
| | | | | GAAGAGGACGACGCUGGAGUUAGCGAAAGAA |
| | | | | CACCGGUGAGUGUGCCACCUCCCACACCGCCA |
| | | | | AGGAGACCCCCAGUAGCACCUCCAACCCAUCC |
| | | | | GAGAGUGAUUCCCGAGGUCAGCCAUGUGCGC |
| | | | | GGCGUAACUGUGCACAUGGAGACGCCCGAAG |
| | | | | CGAUACUGUUUGCCCCUGGAGAGACAUUCGG |
| | | | | CACCAAUGUGUCCAUACACGCAAUUGCGCAC |
| | | | | GAUGAUGGCCCAUACGCUAUGGACGUCGUCU |
| | | | | GGAUGAGGUUCGAUGUGCCUUCUUCUUGCGC |
| | | | | CGAGAUGAGGAUCUACGAGGCAUGCCUGUAU |
| | | | | CACCCCCAAUUGCCGGAGUGUCUGUCUCCCGC |
| | | | | AGAUGCACCGUGUGCAGUGAGUAGCUGGGCU |
| | | | | UAUCGGUUGGCUGUCCGGAGUUAUGCUGGGU |
| | | | | GUUCACGGACCACCCCACCUCCACGUUGCUUU |
| | | | | GCUGAAGCCAGAAUGGAACCCGUGCCUGGUC |
| | | | | UGGCUUGGCUGGCAUCAACUGUCAACCUGGA |
| | | | | GUUCCAGCAUGCCUCUCCACAGCACGCAGGCC |
| | | | | UGUAUCUCUGCGUGGUGUACGUUGACGAUCA |
| | | | | CAUCCAUGCGUGGGGGCAUAUGACCAUCAGC |
| | | | | ACAGCUGCCCAGUACCGCAAUGCCGUCGUGG |
| | | | | AGCAGCACCUCCCCCAACGGCAGCCAGAACCA |
| | | | | GUGGAGCCCACUCGGCCUCAUGUGCGAGCCU |
| | | | | GA |
| 326 | None | HSV-2 | Version | AUGCGCACCUCUUGGAAACGCGUUACUUCCG |
| | | gE | 2 | GGGAGGACGUUGUCCUCCUUCCAGCACCCGC |
| | | | | AGGACCUGAGGAAAGGACUAGGGCCCACAAG |
| | | | | CUGCUGUGGGCCGCUGAACCUCUGGAUGCCU |
| | | | | GUGGUCCUCUGAGACCUAGCUGGGUCGCCCU |
| | | | | UUGGCCACCUAGACGCGUUCUGGAGACGGUC |
| | | | | GUGGAUGCCGCGUGCAUGCGUGCACCCGAAC |
| | | | | CUCUGGCCAUCGCCUAUAGUCCCCCUUUUCCC |
| | | | | GCUGGCGACGAGGGGCUUUACUCCGAACUGG |
| | | | | CCUGGCGGGAUAGGGUGGCGGUGGUGAACGA |
| | | | | GAGCCUCGUCAUCUACGGUGCUCUGGAAACC |
| | | | | GACUCAGGACUGUAUACGCUCAGCGUUGUUG |
| | | | | GCCUCUCCGAUGAGGCUCGACAGGUUGCCUC |
| | | | | CGUAGUGCUGGUCGUAGAACCAGCCCCCGUA |
| | | | | CCAACACCCACACCCGACGACUACGACGAGGA |
| | | | | GGACGACGCUGGAGUUAGCGAAAGAACACCG |
| | | | | GUGAGUGUGCCACCUCCCACACCGCCAAGGA |
| | | | | GACCCCCAGUAGCACCUCCAACCCAUCCGAGA |
| | | | | GUGAUUCCCGAGGUCAGCCAUGUGCGCGGCG |
| | | | | UAACUGUGCACAUGGAGACGCCCGAAGCGAU |
| | | | | ACUGUUUGCCCCUGGAGAGACAUUCGGCACC |
| | | | | AAUGUGUCCAUACACGCAAUUGCGCACGAUG |
| | | | | AUGGCCCAUACGCUAUGGACGUCGUCUGGAU |
| | | | | GAGGUUCGAUGUGCCUUCUUCUUGCGCCGAG |
| | | | | AUGAGGAUCUACGAGGCAUGCCUGUAUCACC |
| | | | | CCCAAUUGCCGGAGUGUCUGUCUCCCGCAGA |
| | | | | UGCACCGUGUGCAGUGAGUAGCUGGGCUUAU |
| | | | | CGGUUGGCUGUCCGGAGUUAUGCUGGGUGUU |
| | | | | CACGGACCACCCCACCUCCACGUUGCUUUGCU |
| | | | | GAAGCCAGAAUGGAACCCGUGCCUGGUCUGG |
| | | | | CUUGGCUGGCAUCAACUGUCAACCUGGAGUU |
| | | | | CCAGCAUGCCUCUCCACAGCACGCAGGCCUGU |
| | | | | AUCUCUGCGUGGUGUACGUUGACGAUCACAU |
| | | | | CCAUGCGUGGGGGCAUAUGACCAUCAGCACA |
| | | | | GCUGCCCAGUACCGCAAUGCCGUCGUGGAGC |
| | | | | AGCACCUCCCCCAACGGCAGCCAGAACCAGUG |
| | | | | GAGCCCACUCGGCCUCAUGUGCGAGCCUGA |
| 209 | HSV-1 | HSV-2 | Version | AUGGGCGGAGCUGCUGCUAGACUGGGAGCCG |
| | gD | gE | 1 | UGAUCCUGUUCGUGGUUAUCGUGGGACUGCA |
| | | | | UGGCGUGCGGGGCAAGUAUAGAACCUCUUGG |
| | | | | AAGCGCGUGACAAGCGGCGAGGAUGUGGUUC |
| | | | | UGCUUCCUGCUCCUGCCGGACCUGAGGAAAG |
| | | | | AACAAGAGCCCACAAGCUGCUGUGGGCCGCU |
| | | | | GAACCUUUGGAUGCCUGUGGACCUCUGAGGC |
| | | | | CUUCUUGGGUUGCACUGUGGCCACCUCGGAG |
| | | | | AGUGCUGGAAACAGUGGUGGAUGCCGCCUGC |
| | | | | AUGAGAGCCCCUGAACCUCUGGCCAUUGCCU |
| | | | | ACUCUCCACCUUUUCCAGCCGGCGACGAGGG |
| | | | | CCUGUAUUCUGAGCUUGCUUGGAGAGACAGA |
| | | | | GUGGCCGUGGUCAACGAGAGCCUGGUUAUCU |
| | | | | AUGGCGCCCUGGAAACCGACAGCGGCCUGUA |
| | | | | CACACUGUCUGUCGUGGGCCUGUCUGACGAG |
| | | | | GCUAGACAGGUGGCAUCUGUGGUGCUGGUGG |
| | | | | UGGAACCUGCUCCAGUGCCUACACCUACACC |
| | | | | UGACGACUACGACGAGGAAGAUGACGCUGGC |
| | | | | GUCAGCGAGAGAACCCCUGUUUCUGUGCCUC |
| | | | | CUCCUACGCCUCCUCGUAGACCUCCUGUUGCU |
| | | | | CCUCCAACACACCCCAGAGUGAUCCCUGAAG |
| | | | | UGUCUCACGUGCGGGGCGUGACCGUGCACAU |
| | | | | GGAAACACCUGAGGCCAUCCUGUUCGCCCCU |
| | | | | GGCGAGACAUUUGGCACCAACGUGUCCAUCC |
| | | | | ACGCUAUCGCCCACGACGAUGGCCCUUACGCC |
| | | | | AUGGAUGUCGUGUGGAUGAGAUUCGACGUGC |
| | | | | CCAGCAGCUGCGCCGAGAUGAGAAUCUACGA |
| | | | | GGCCUGCCUGUAUCACCCUCAGCUGCCCGAA |
| | | | | UGUCUGAGCCCUGCUGAUGCCCCUUGUGCCG |
| | | | | UUAGCAGCUGGGCCUAUAGACUGGCCGUGCG |
| | | | | GUCUUAUGCCGGCUGCUCUAGAACAACCCCU |
| | | | | CCUCCUCGGUGUUUCGCCGAGGCCAGAAUGG |
| | | | | AACCUGUUCCUGGACUGGCCUGGCUGGCCUC |
| | | | | CACAGUGAACCUGGAAUUUCAGCACGCCUCU |
| | | | | CCACAGCACGCCGGCCUGUAUCUGUGUGUGG |
| | | | | UGUACGUGGACGAUCACAUCCACGCCUGGGG |
| | | | | CCACAUGACCAUCUCUACAGCCGCUCAGUACC |
| | | | | GGAACGCCGUGGUUGAACAGCAUCUGCCCCA |
| | | | | GAGACAGCCCGAGCCUGUGGAACCUACAAGA |
| | | | | CCUCAUGUUCGGGCCUGAUAA |
| 210 | HSV-1 | HSV-2 | Version | AUGGGCGGAGCUGCGGCUCGCCUCGGAGCCG |
| | gD | gE | 2 | UCAUUCUGUUCGUGGUGAUCGUGGGUCUGCA |
| | | | | UGGGGUCAGAGGAAAGUACCGGACCAGCUGG |
| | | | | AAAAGGGUGACAUCUGGGGAAGAUGUCGUGC |
| | | | | UCCUUCCUGCGCCUGCUGGCCCAGAAGAACG |
| | | | | CACUAGGGCCCACAAGCUUCUGUGGGCCGCG |
| | | | | GAGCCACUGGAUGCGUGUGGUCCCCUGCGAC |
| | | | | CCUCUUGGGUGGCAUUGUGGCCACCCCGACG |
| | | | | CGUACUCGAAACGGUCGUUGACGCCGCCUGU |
| | | | | AUGAGAGCGCCAGAGCCCCUCGCCAUUGCCU |
| | | | | ACAGCCCGCCUUUUCCCGCCGGUGACGAAGG |
| | | | | ACUGUACAGUGAGCUGGCCUGGCGCGAUAGG |
| | | | | GUCGCCGUAGUUAACGAGUCCCUGGUGAUAU |
| | | | | ACGGCGCUCUGGAAACCGACAGUGGCUUGUA |
| | | | | CACCCUGUCCGUUGUAGGCCUGUCCGAUGAA |
| | | | | GCACGGCAAGUGGCUUCCGUGGUACUGGUCG |
| | | | | UAGAGCCCGCACCAGUUCCCACACCCACCCCG |
| | | | | GAUGACUACGAUGAGGAGGACGAUGCCGGUG |
| | | | | UGAGUGAGCGUACACCUGUUAGCGUACCUCC |
| | | | | ACCAACUCCUCCACGCCGCCCUCCUGUUGCAC |
| | | | | CGCCUACACAUCCCCGUGUGAUUCCUGAAGU |
| | | | | GUCACACGUUAGAGGGGUGACAGUCCACAUG |
| | | | | GAGACUCCCGAAGCCAUCCUCUUUGCACCAG |
| | | | | GCGAGACUUUUGGGACCAAUGUGAGCAUUCA |
| | | | | CGCCAUAGCUCACGAUGACGGGCCCUAUGCC |
| | | | | AUGGACGUGGUGUGGAUGAGGUUCGAUGUGC |
| | | | | CCUCAUCAUGCGCUGAGAUGCGGAUCUACGA |
| | | | | AGCUUGCCUGUAUCACCCACAGCUUCCCGAG |
| | | | | UGCUUGUCUCCCGCUGACGCCCCUUGUGCUG |
| | | | | UUAGCUCUUGGGCUUAUCGGCUUGCCGUCAG |
| | | | | GAGCUAUGCUGGAUGCUCCAGAACCACACCU |
| | | | | CCACCGAGGUGUUUCGCCGAGGCCAGAAUGG |
| | | | | AGCCUGUGCCAGGACUGGCCUGGCUGGCAAG |
| | | | | UACUGUGAACCUGGAAUUCCAGCACGCAUCA |
| | | | | CCUCAGCAUGCAGGCCUGUACCUCUGCGUUG |
| | | | | UCUAUGUCGACGACCAUAUCCACGCAUGGGG |
| | | | | CCAUAUGACCAUCAGCACGGCAGCACAGUAU |
| | | | | CGGAAUGCUGUGGUCGAGCAGCACUUGCCCC |
| | | | | AGCGACAACCCGAACCAGUGGAGCCAACCAG |
| | | | | ACCGCAUGUGCGGGCCUGA |
| 211 | HSV-1 | HSV-2 | Version | AUGGGAGGAGCUGCUGCUAGAUUGGGAGCUG |
| | gD | gE | 3 | UGAUUCUGUUUGUGGUGAUUGUGGGACUGCA |
| | | | | UGGAGUGAGAGGAAAAUACAGAACCUCUUGG |
| | | | | AAAAGAGUGACCUCUGGAGAAGAUGUGGUGC |
| | | | | UGCUGCCAGCUCCAGCUGGACCAGAAGAAAG |
| | | | | AACCAGAGCACACAAACUGCUGUGGGCUGCU |
| | | | | GAACCUCUGGAUGCUUGUGGACCUCUGAGAC |
| | | | | CUUCUUGGGUGGCUCUGUGGCCACCAAGAAG |
| | | | | GGUGCUGGAAACAGUGGUGGAUGCUGCUUGC |
| | | | | AUGAGAGCACCUGAACCUCUGGCAAUUGCAU |
| | | | | ACUCUCCUCCUUUUCCAGCUGGAGAUGAAGG |
| | | | | ACUGUAUUCUGAACUGGCUUGGAGAGACAGA |
| | | | | GUGGCUGUGGUGAAUGAAUCUCUGGUGAUCU |
| | | | | AUGGAGCACUGGAAACAGAUUCUGGACUGUA |
| | | | | CACCCUGUCUGUGGUGGGACUGUCUGAUGAA |
| | | | | GCAAGACAGGUGGCAUCUGUGGUGCUGGUGG |
| | | | | UGGAACCAGCUCCAGUGCCAACCCCAACCCCA |
| | | | | GAUGAUUAUGAUGAAGAAGAUGAUGCUGGAG |
| | | | | UGUCUGAAAGAACCCCAGUGUCUGUGCCACC |
| | | | | ACCAACCCCACCAAGAAGACCACCAGUGGCUC |
| | | | | CACCAACCCACCCAAGAGUGAUCCCAGAAGU |
| | | | | GUCUCAUGUGAGAGGAGUGACAGUGCACAUG |
| | | | | GAAACCCCUGAAGCAAUCCUGUUUGCACCUG |
| | | | | GAGAAACCUUUGGAACCAAUGUGUCCAUCCA |
| | | | | UGCAAUUGCACAUGAUGAUGGACCUUAUGCA |
| | | | | AUGGAUGUGGUGUGGAUGAGAUUUGAUGUGC |
| | | | | CUUCUUCUUGUGCUGAAAUGAGAAUCUAUGA |
| | | | | AGCUUGCCUGUACCACCCUCAGCUGCCUGAA |
| | | | | UGCCUGUCUCCUGCUGAUGCUCCUUGUGCUG |
| | | | | UGUCUUCUUGGGCUUACAGACUGGCUGUGAG |
| | | | | AUCUUAUGCUGGAUGCUCCAGAACCACCCCA |
| | | | | CCACCAAGAUGCUUUGCUGAAGCAAGAAUGG |
| | | | | AACCUGUGCCUGGACUGGCAUGGCUGGCAUC |
| | | | | CACAGUGAAUCUGGAAUUUCAGCAUGCUUCU |
| | | | | CCUCAGCAUGCUGGACUGUACCUGUGUGUGG |
| | | | | UGUAUGUGGACGAUCACAUCCAUGCUUGGGG |
| | | | | ACACAUGACCAUCAGCACAGCUGCUCAGUAC |
| | | | | AGAAAUGCUGUGGUGGAACAGCACCUGCCAC |
| | | | | AGAGACAGCCAGAACCAGUGGAACCAACCAG |
| | | | | ACCACAUGUGAGAGCUUGAUAA |
| 212 | HSV-1 | HSV-2 | Version | AUGGGCGGAGCUGCGGCUCGCCUCGGAGCCG |
| | gD | gE | 4 | UCAUUCUGUUCGUGGUGAUCGUGGGUCUGCA |
| | | | | UGGGGUCAGAGGAAAGUACCGGACCAGCUGG |
| | | | | AAAAGGGUGACAUCUGGGGAAGAUGUCGUGC |
| | | | | UCCUUCCUGCGCCUGCUGGCCCAGAAGAACG |
| | | | | CACUAGGGCCCACAAGCUUCUGUGGGCCGCG |
| | | | | GAGCCACUGGAUGCGUGUGGUCCCCUGCGAC |
| | | | | CCUCUUGGGUGGCAUUGUGGCCACCCCGACG |
| | | | | CGUACUCGAAACGGUCGUUGACGCCGCCUGU |
| | | | | AUGAGAGCGCCAGAGCCCCUCGCCAUUGCCU |
| | | | | ACAGCCCGCCUUUUCCCGCCGGUGACGAAGG |
| | | | | ACUGUACAGUGAGCUGGCCUGGCGCGAUAGG |
| | | | | GUCGCCGUAGUUAACGAGUCCCUGGUGAUAU |
| | | | | ACGGCGCUCUGGAAACCGACAGUGGCUUGUA |
| | | | | CACCCUGUCCGUUGUAGGCCUGUCCGAUGAA |
| | | | | GCACGGCAAGUGGCUUCCGUGGUACUGGUCG |
| | | | | UAGAGCCCGCACCAGUUCCCACACCCACCCCG |
| | | | | GAUGACUACGAUGAGGAGGACGAUGCCGGUG |
| | | | | UGAGUGAGCGUACACCUGUUAGCGUACCUCC |
| | | | | ACCAACUCCUCCACGCCGCCCUCCUGUUGCAC |
| | | | | CGCCUACACAUCCCCGUGUGAUUCCUGAAGU |
| | | | | GUCACACGUUAGAGGGGUGACAGUCCACAUG |
| | | | | GAGACUCCCGAAGCCAUCCUCUUUGCACCAG |
| | | | | GCGAGACUUUUGGGACCAAUGUGAGCAUUCA |
| | | | | CGCCAUAGCUCACGAUGACGGGCCCUAUGCC |
| | | | | AUGGACGUGGUGUGGAUGAGGUUCGAUGUGC |
| | | | | CCUCAUCAUGCGCUGAGAUGCGGAUCUACGA |
| | | | | AGCUUGCCUGUAUCACCCACAGCUUCCCGAG |
| | | | | UGCUUGUCUCCCGCUGACGCCCCUUGUGCUG |
| | | | | UUAGCUCUUGGGCUUAUCGGCUUGCCGUCAG |
| | | | | GAGCUAUGCUGGAUGCUCCAGAACCACACCU |
| | | | | CCACCGAGGUGUUUCGCCGAGGCCAGAAUGG |
| | | | | AGCCUGUGCCAGGACUGGCCUGGCUGGCAAG |
| | | | | UACUGUGAACCUGGAAUUCCAGCACGCAUCA |
| | | | | CCUCAGCAUGCAGGCCUGUACCUCUGCGUUG |
| | | | | UCUAUGUCGACGACCAUAUCCACGCAUGGGG |
| | | | | CCAUAUGACCAUCAGCACGGCAGCACAGUAU |
| | | | | CGGAAUGCUGUGGUCGAGCAGCACUUGCCCC |
| | | | | AGCGACAACCCGAACCAGUGGAGCCAACCAG |
| | | | | ACCGCAUGUGCGGGCCUGA |

### D. Transmembrane Regions

In some embodiments, a polypeptide described herein includes a transmembrane region. In some embodiments, a polyribonucleotide described herein encodes a polypeptide that comprises a transmembrane region. In some embodiments, a transmembrane region is located at the N-terminus of a polypeptide. In some embodiments, a transmembrane region is located at the C-terminus of a polypeptide. In some embodiments, a transmembrane region is not located at the N-terminus or C-terminus of a polypeptide. In some embodiments, a polypeptide does not include a transmembrane region.

Transmembrane regions are known in the art, any of which can be utilized in a polypeptide described herein. In some embodiments, a transmembrane region comprises or is a transmembrane region of Hemagglutinin (HA) of Influenza virus, Env of HIV-1, equine infectious anaemia virus (EIAV), murine leukaemia virus (MLV), mouse mammary tumor virus, G protein of vesicular stomatitis virus (VSV), Rabies virus, or a seven transmembrane domain receptor. In some embodiments, a polypeptide comprises an HSV transmembrane region. In some embodiments, an HSV transmembrane region is an HSV-1 or HSV-2 transmembrane region. In some embodiments, an HSV transmembrane region is an HSV-2 gD transmembrane region. In some embodiments, an HSV transmembrane region is an HSV-2 gC transmembrane region. In some embodiments, an HSV transmembrane region is an HSV-2 gE transmembrane region.

### III. Polyribonucleotides

### A. Example Polyribonucleotides Features

The present disclosure also provides RNA constructs comprising polyribonucleotides described herein. In some embodiments, an RNA construct provided herein comprises a polyribonucleotide that encodes an HSV-2 gC antigen or antigenic fragment thereof. In some embodiments, an RNA construct provided herein comprises a polyribonucleotide that encodes an HSV-2 gD antigen or antigenic fragment thereof. In some embodiments, an RNA construct provided herein comprises a polyribonucleotide that encodes an HSV-2 gE antigen or antigenic fragment thereof.

In some embodiments, an RNA construct provided herein comprises a polyribonucleotide that encodes an HSV-2 gC antigen. In some embodiments, an RNA construct provided herein comprises a polyribonucleotide that encodes an HSV-2 gD antigen. In some embodiments, an RNA construct provided herein comprises a polyribonucleotide that encodes an HSV-2 gE antigen.

In some embodiments, an RNA construct provided herein comprises a polyribonucleotide that encodes an HSV-2 gC antigen and a secretory signal. In some embodiments, an RNA construct provided herein comprises a polyribonucleotide that encodes an HSV-2 gD antigen and a secretory signal. In some embodiments, an RNA construct provided herein comprises a polyribonucleotide that encodes an HSV-2 gE antigen and a secretory signal.

In some embodiments, polyribonucleotides described herein can comprise a nucleotide sequence that encodes a 5′UTR and/or a 3′ UTR. In some embodiments, polynucleotides described herein can comprise a nucleotide sequence that encodes a polyA tail. In some embodiments, polyribonucleotides described herein may comprise a 5′ cap, which may be incorporated during transcription, or joined to a polyribonucleotide post-transcription.

1. 5′ Cap

A structural feature of RNAs is cap structure at five-prime end (5′). Natural eukaryotic RNA comprises a 7-methylguanosine cap linked to the RNA via a 5′ to 5′-triphosphate bridge resulting in cap0 structure (m7GpppN). In most eukaryotic RNA and some viral RNA, further modifications can occur at the 2′-hydroxy-group (2′-OH) (e.g., the 2′-hydroxyl group may be methylated to form 2′-O-Me) of the first and subsequent nucleotides producing “cap1” and “cap2” five-prime ends, respectively). Diamond, et al., (2014) Cytokine & growth Factor Reviews, 25:543-550, which is incorporated herein by reference in its entirety, reported that cap0-RNA cannot be translated as efficiently as cap1-RNA in which the role of 2′-O-Me in the penultimate position at the RNA 5′ end is determinant. Lack of the 2′-O-met has been shown to trigger innate immunity and activate IFN response. Daffis, et al. (2010) Nature, 468:452-456; and Züst et al. (2011) Nature Immunology, 12:137-143, each of which is incorporated herein by reference in its entirety.

RNA capping is well researched and is described, e.g., in Decroly E et al. (2012) Nature Reviews 10:51-65; and in Ramanathan A. et al., (2016) Nucleic Acids Res; 44 (16): 7511-7526, the entire contents of each of which is hereby incorporated by reference. For example, in some embodiments, a 5′-cap structure which may be suitable in the context of the present invention is a cap0 (methylation of the first nucleobase, e.g., m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (“anti-reverse cap analogue”), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

The term “5′-cap” as used herein refers to a structure found on the 5′-end of an RNA, e.g., mRNA, and generally includes a guanosine nucleotide connected to an RNA, e.g., mRNA, via a 5′- to 5′-triphosphate linkage (also referred to as Gppp or G(5′)ppp(5′)). In some embodiments, a guanosine nucleoside included in a 5′ cap may be modified, for example, by methylation at one or more positions (e.g., at the 7-position) on a base (guanine), and/or by methylation at one or more positions of a ribose. In some embodiments, a guanosine nucleoside included in a 5′ cap comprises a 3′O methylation at a ribose (3′OMeG). In some embodiments, a guanosine nucleoside included in a 5′ cap comprises methylation at the 7-position of guanine (m7G). In some embodiments, a guanosine nucleoside included in a 5′ cap comprises methylation at the 7-position of guanine and a 3′ O methylation at a ribose (m7(3′OMeG)). It will be understood that the notation used in the above paragraph, e.g., “(m₂^{7,3′-O})G” or “m7(3′OMeG)”, applies to other structures described herein.

In some embodiments, providing an RNA with a 5′-cap disclosed herein may be achieved by in vitro transcription, in which a 5′-cap is co-transcriptionally expressed into an RNA strand, or may be attached to an RNA post-transcriptionally using capping enzymes. In some embodiments, co-transcriptional capping with a cap disclosed improves the capping efficiency of an RNA compared to co-transcriptional capping with an appropriate reference comparator. In some embodiments, improving capping efficiency can increase a translation efficiency and/or translation rate of an RNA, and/or increase expression of an encoded polypeptide. In some embodiments, alterations to polynucleotides generates a non-hydrolyzable cap structure which can, for example, prevent decapping and increase RNA half-life.

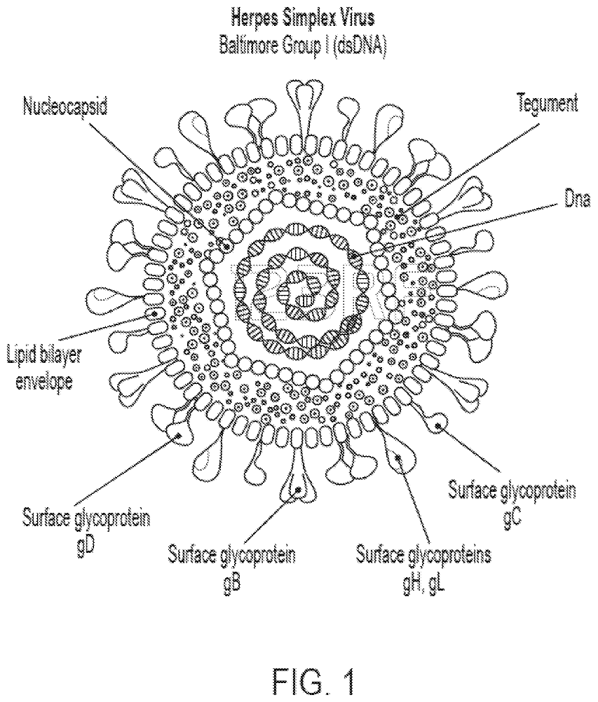

In some embodiments, a utilized 5′ caps is a cap0, a cap1, or cap2 structure. See, e.g., FIG. 1 of Ramanathan A et al., and FIG. 1 of Decroly E et al., each of which is incorporated herein by reference in its entirety. See, e.g., FIG. 1 of Ramanathan A et al., and FIG. 1 of Decroly E et al., each of which is incorporated herein by reference in its entirety. In some embodiments, an RNA described herein comprises a cap1 structure. In some embodiments, an RNA described herein comprises a cap2.

In some embodiments, an RNA described herein comprises a cap0 structure. In some embodiments, a cap0 structure comprises a guanosine nucleoside methylated at the 7-position of guanine ((m2)G). In some embodiments, such a cap0 structure is connected to an RNA via a 5′- to 5′-triphosphate linkage and is also referred to herein as (m7)Gppp. In some embodiments, a cap0 structure comprises a guanosine nucleoside methylated at the 2′-position of the ribose of guanosine. In some embodiments, a cap0 structure comprises a guanosine nucleoside methylated at the 3′-position of the ribose of guanosine. In some embodiments, a guanosine nucleoside included in a 5′ cap comprises methylation at the 7-position of guanine and at the 2′-position of the ribose ((m₂^{7,2′-O})G). In some embodiments, a guanosine nucleoside included in a 5′ cap comprises methylation at the 7-position of guanine and at the 2′-position of the ribose ((m₂^{7,3′-O})G).

In some embodiments, a cap1 structure comprises a guanosine nucleoside methylated at the 7-position of guanine ((m⁷)G) and optionally methylated at the 2′ or 3′ position pf the ribose, and a 2′O methylated first nucleotide in an RNA ((m^{2′-O})N₁). In some embodiments, a cap1 structure comprises a guanosine nucleoside methylated at the 7-position of guanine ((m⁷)G) and the 3′ position of the ribose, and a 2′O methylated first nucleotide in an RNA ((m^{2′-O})N₁). In some embodiments, a cap1 structure is connected to an RNA via a 5′- to 5′-triphosphate linkage and is also referred to herein as, e.g., ((m⁷)Gppp(^{2′-O})N₁) or (m₂^{7,3′-O})Gppp(^{2′-O})N₁), wherein N₁ is as defined and described herein. In some embodiments, a cap1 structure comprises a second nucleotide, N₂, which is at position 2 and is chosen from A, G, C, or U, e.g., (m⁷)Gppp(^{2′-O})N₁pN₂ or (m₂^{7,3′-O})Gppp(^{2′-O})N₁pN₂, wherein each of N₁ and N₂ is as defined and described herein.

In some embodiments, a cap2 structure comprises a guanosine nucleoside methylated at the 7-position of guanine ((m⁷)G) and optionally methylated at the 2′ or 3′ position of the ribose, and a 2′O methylated first and second nucleotides in an RNA ((m^{2′-O})N₁p(m^{2′-O})N₂). In some embodiments, a cap2 structure comprises a guanosine nucleoside methylated at the 7-position of guanine ((m⁷)G) and the 3′ position of the ribose, and a 2′O methylated first and second nucleotide in an RNA. In some embodiments, a cap2 structure is connected to an RNA via a 5′- to 5′-triphosphate linkage and is also referred to herein as, e.g., ((m⁷)Gppp(^{2′-O})N₁p(^{2′-O})N₂) or (m₂^{7,3′-O})Gppp(^{2′-O})N₁p(^{2′-O})N₂), wherein each of N₁ and N₂ is as defined and described herein.

In some embodiments, the 5′ cap is a dinucleotide cap structure. In some embodiments, the 5′ cap is a dinucleotide cap structure comprising N₁, wherein N₁ is as defined and described herein. In some embodiments, the 5′ cap is a dinucleotide cap G*N₁, wherein N₁ is as defined above and herein, and G* comprises a structure of formula (I):

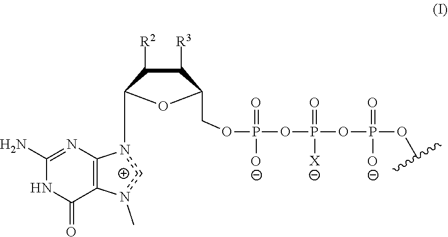

or a salt thereof,
wherein each R² and R³ is —OH or −OCH₃; and X is O or S.

In some embodiments, R² is —OH. In some embodiments, R² is —OCH₃. In some embodiments, R³ is —OH. In some embodiments, R³ is —OCH₃. In some embodiments, R² is —OH and R³ is —OH. In some embodiments, R² is —OH and R³ is —CH₃. In some embodiments, R² is —CH₃ and R³ is —OH. In some embodiments, R² is —CH₃ and R³ is —CH₃.

In some embodiments, X is O. In some embodiments, X is S.

In some embodiments, the 5′ cap is a dinucleotide cap0 structure (e.g., (m⁷)GpppN₁, (m₂^{7,2′-O})GpppN₁, (m₂^{7,3′-O})GpppN₁, (m⁷)GppSpN₁, (m₂^{7,2′-O})GppSpN₁, or (m₂^{7,3′-O})GppSpN₁), wherein N₁ is as defined and described herein. In some embodiments, the 5′ cap is a dinucleotide cap0 structure (e.g., (m⁷)GpppN₁, (m₂^{7,2′-O})GpppN₁, (m₂^{7,3′-O})GpppN₁, (m⁷)GppSpN₁, (m₂^{7,2′-O})GppSpN₁, or (m₂^{7,3′-O})GppSpN₁), wherein N₁ is G. In some embodiments, the 5′ cap is a dinucleotide cap0 structure (e.g., (m⁷)GpppN₁, (m₂^{7,2′-O})GpppN₁, (m₂^{7,3′-O})GpppN₁, (m⁷)GppSpN₁, (m₂^{7,2′-O})GppSpN₁, or (m₂^{7,3′-O})GppSpN₁), wherein N₁ is A, U, or C. In some embodiments, the 5′ cap is a dinucleotide cap1 structure (e.g., (m⁷)Gppp(m^{2′-O})N₁, (m₂^{7,2′-O})Gppp(m^{2′-O})N₁, (m₂^{7,3′-O})Gppp(m^{2′-O})N₁, (m⁷)GppSp(m^{2′-O})N₁, (m₂^{7,2′-O})GppSp (m^{2′-O})N₁, or (m₂^{7,3′-O})GppSp(m^{2′-O})N₁), wherein N₁ is as defined and described herein. In some embodiments, the 5′ cap is selected from the group consisting of (m⁷)GpppG(“Ecap0”), (m⁷)Gppp(m^{2′-O})G (“Ecap1”), (m₂^{7,3′-O})GpppG(“ARCA” or “D1”), and (m₂^{7,2′-O})GppSpG (“beta-S-ARCA”). In some embodiments, the 5′ cap is (m⁷)GpppG(“Ecap0”), having a structure:

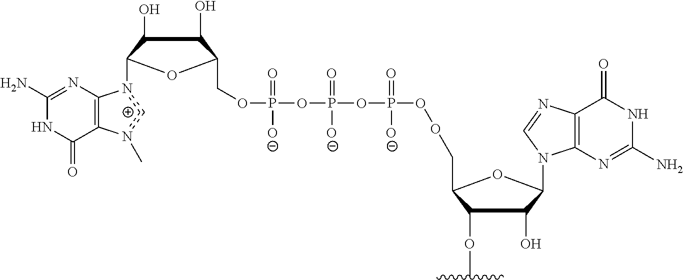

or a salt thereof.

In some embodiments, the 5′ cap is (m⁷)Gppp(m^{2′O})G (“Ecap1”), having a structure:

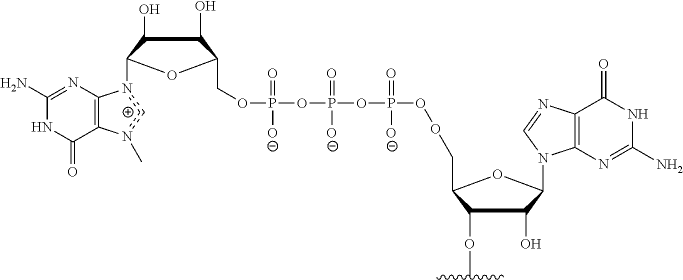

or a salt thereof.

In some embodiments, the 5′ cap is (m₂^{7,3′-O})GpppG(“ARCA” or “D1”), having a structure:

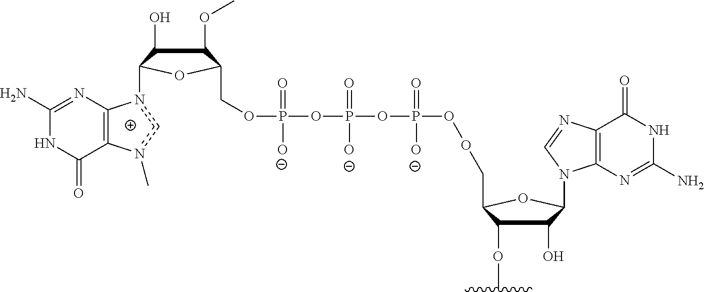

or a salt thereof.

In some embodiments, the 5′ cap is (m₂^{7,2′-O})GppSpG (“beta-S-ARCA”), having a structure:

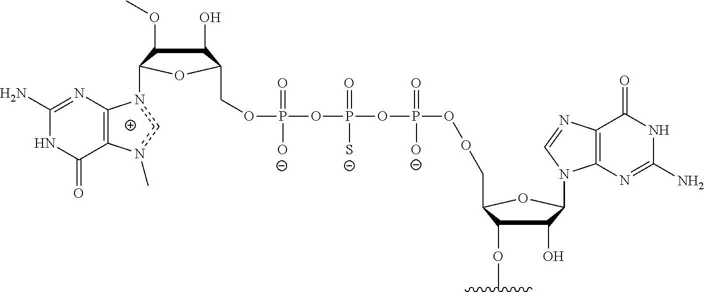

or a salt thereof.

In some embodiments, the 5′ cap is a trinucleotide cap structure. In some embodiments, the 5′ cap is a trinucleotide cap structure comprising N₁pN₂, wherein N₁ and N₂ are as defined and described herein. In some embodiments, the 5′ cap is a dinucleotide cap G*N₁pN₂, wherein N₁ and N₂ are as defined above and herein, and G* comprises a structure of formula (I):

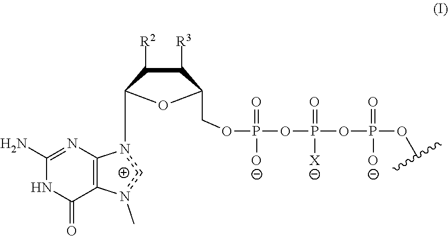

or a salt thereof, wherein R², R³, and X are as defined and described herein.

In some embodiments, the 5′ cap is a trinucleotide cap0 structure (e.g. (m⁷)GpppN₁pN₂, (m₂^{7,2′-O})GpppN₁pN₂, or (m₂^{7,3′-O})GpppN₁pN₂), wherein N₁ and N₂ are as defined and described herein). In some embodiments, the 5′ cap is a trinucleotide cap1 structure (e.g., (m⁷)Gppp(m^{2′-O})N₁pN₂, (m₂^{7,2′-O})Gppp(m^{2′-O})N₁pN₂, (m₂^{7,3′-O})Gppp(m^{2′-O})N₁pN₂), wherein N₁ and N₂ are as defined and described herein. In some embodiments, the 5′ cap is a trinucleotide cap2 structure (e.g., (m⁷)Gppp(m^{2′-O})N₁p(m^{2′-O})N₂, (m₂^{7,2′-O})Gppp(m^{2′-O})N₁p(m^{2′-O})N₂, (m₂^{7,3′-O})Gppp(m^{2′-O})N₁p(m^{2′-O})N₂), wherein N₁ and N₂ are as defined and described herein. In some embodiments, the 5′ cap is selected from the group consisting of (m₂^{7,3′-O})Gppp(m^{2′O})ApG(“CleanCap AG”, “CC413”), (m₂^{7,3′-O})Gppp(m^{2′O})GpG(“CleanCap GG”), (m⁷)Gppp(m^{2′-O})ApG, (m⁷)Gppp(m^{2′-O})GpG, (m₂^{7,3′-O})Gppp(m₂^{6,2′-O})ApG, and (m⁷)Gppp(m^{2′-O})ApU.

In some embodiments, the 5′ cap is (m₂^{7,3′-O})Gppp(m^{2′-O})ApG(“CleanCap AG”, “CC413”), having a structure:

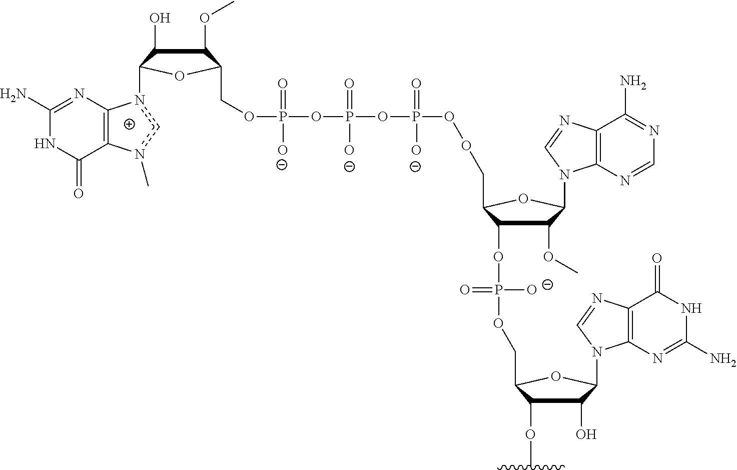

or a salt thereof.

In some embodiments, the 5′ cap is (m₂^{7,3′-O})Gppp(m^{2′-O})GpG(“CleanCap GG”), having a structure:

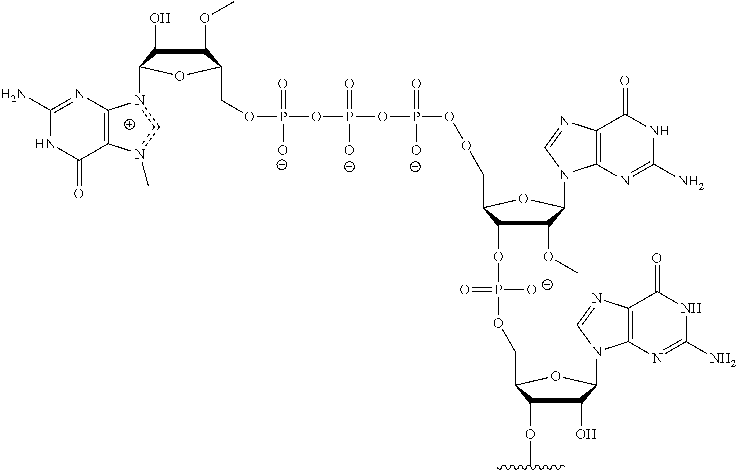

or a salt thereof.

In some embodiments, the 5′ cap is (m⁷)Gppp(m^{2′-O})ApG, having a structure:

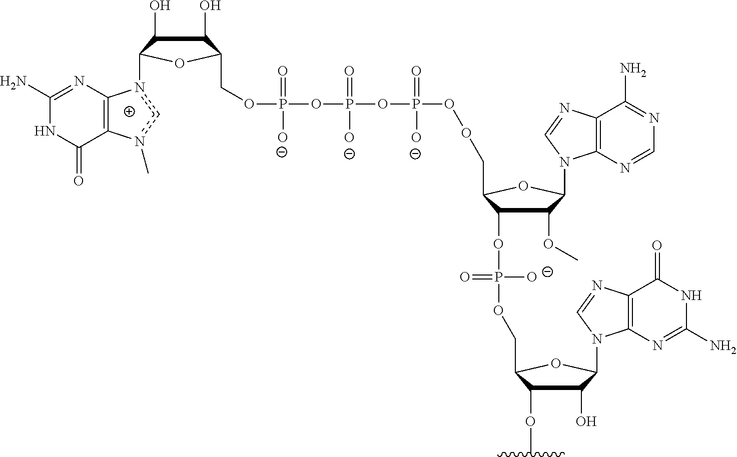

or a salt thereof.

In some embodiments, the 5′ cap is (m⁷)Gppp(m^{2′-O})GpG, having a structure:

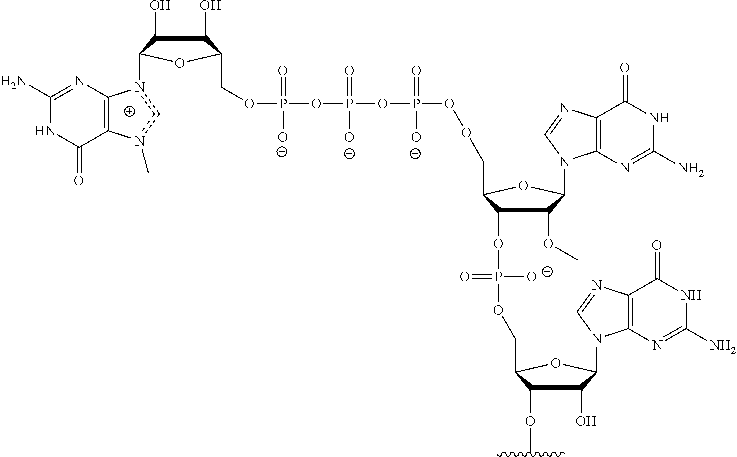

or a salt thereof.

In some embodiments, the 5′ cap is (m₂^{7,3′-O})Gppp(m₂^{6,2′-O})ApG, having a structure:

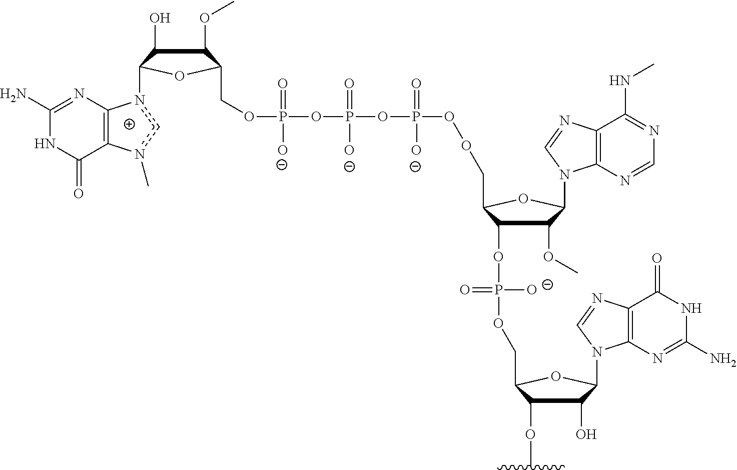

or a salt thereof.

In some embodiments, the 5′ cap is (m⁷)Gppp(m^{2′-O})ApU, having a structure:

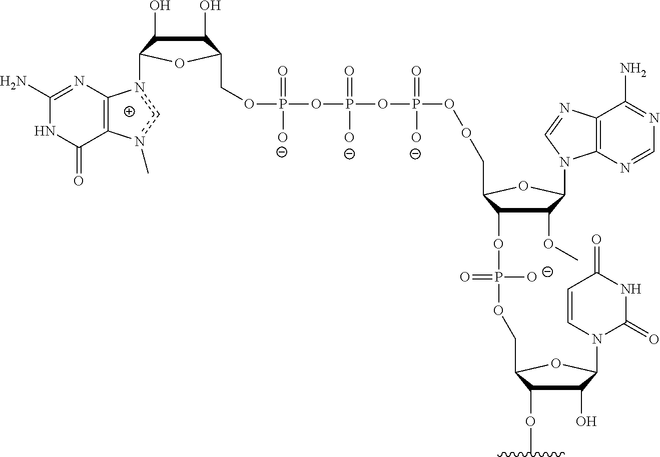

or a salt thereof.

In some embodiments, the 5′ cap is a tetranucleotide cap structure. In some embodiments, the 5′ cap is a tetranucleotide cap structure comprising N₁pN₂pN₃, wherein N₁, N₂, and N₃ are as defined and described herein. In some embodiments, the 5′ cap is a tetranucleotide cap G*N₁pN₂pN₃, wherein N₁, N₂, and N₃ are as defined above and herein, and G* comprises a structure of formula (I):

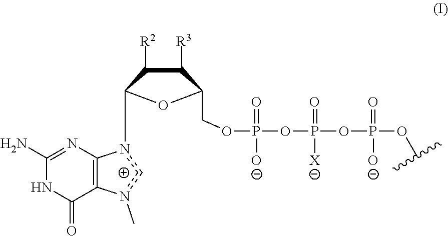

or a salt thereof, wherein R², R³, and X are as defined and described herein.

In some embodiments, the 5′ cap is a tetranucleotide cap0 structure (e.g. (m⁷)GpppN₁pN₂pN₃, (m₂^{7,2′-O})GpppN₁pN2pN₃, or (m₂^{7,3′-O})GpppN₁N₂pN₃), wherein N₁, N₂, and N₃ are as defined and described herein). In some embodiments, the 5′ cap is a tetranucleotide Cap1 structure (e.g., (m⁷)Gppp(m^{2′-O})N₁pN₂PN3, (m₂^{7,2′-O})Gppp(m^{2′-O})N₁pN₂PN3, (m₂^{7,3′-O})Gppp(m^{2′-O})N₁pN₂N₃), wherein N₁, N₂, and N₃ are as defined and described herein. In some embodiments, the 5′ cap is a tetranucleotide Cap2 structure (e.g., (m⁷)Gppp(m^{2′-O}) N₁p(m^{2′-O})N₂PN3, (m₂^{7,2′-O})Gppp(m^{2′-O}) N₁p(m^{2′-O})N₂pN₃, (m₂^{7,3′-O})Gppp(m^{2′-O})N₁p(m^{2′-O})N₂PN3), wherein N₁, N₂, and N₃ are as defined and described herein. In some embodiments, the 5′ cap is selected from the group consisting of (m₂^{7,3′-O})Gppp(m^{2′-O})Ap(m^{2′O})GpG, (m₂^{7,3′-O})Gppp(m^{2′-O})Gp(m^{2′-O})GpC, (m⁷)Gppp(m^{2′-O})Ap(m^{2′-O})UpA, and (m⁷)Gppp(m^{2′-O})Ap(m^{2′-O})GpG.

In some embodiments, the 5′ cap is (m₂^{7,3′-O})Gppp(m^{2′-O})Ap(m^{2′-O})GpG, having a structure:

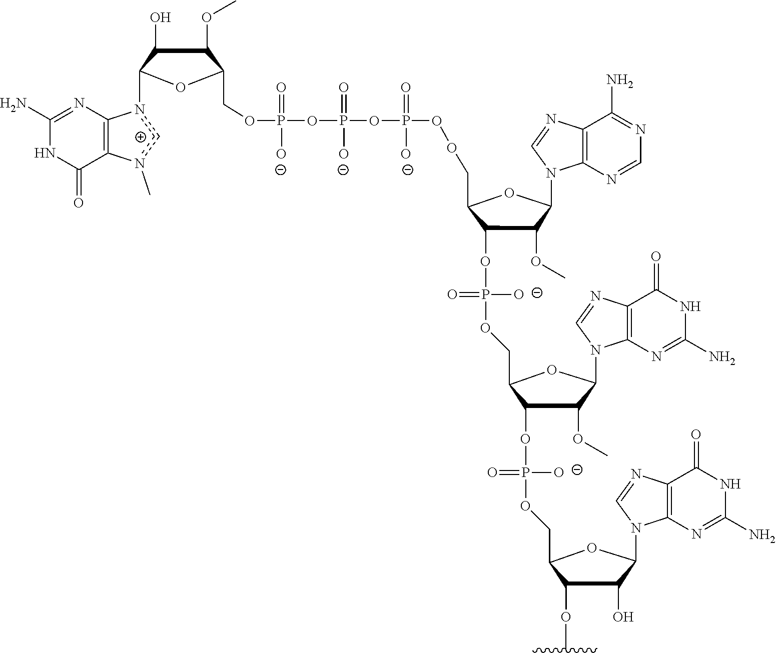

or a salt thereof.

In some embodiments, the 5′ cap is (m₂^{7,3′-O})Gppp(m^{2′-O})Gp(m^{2′-O})GpC, having a structure:

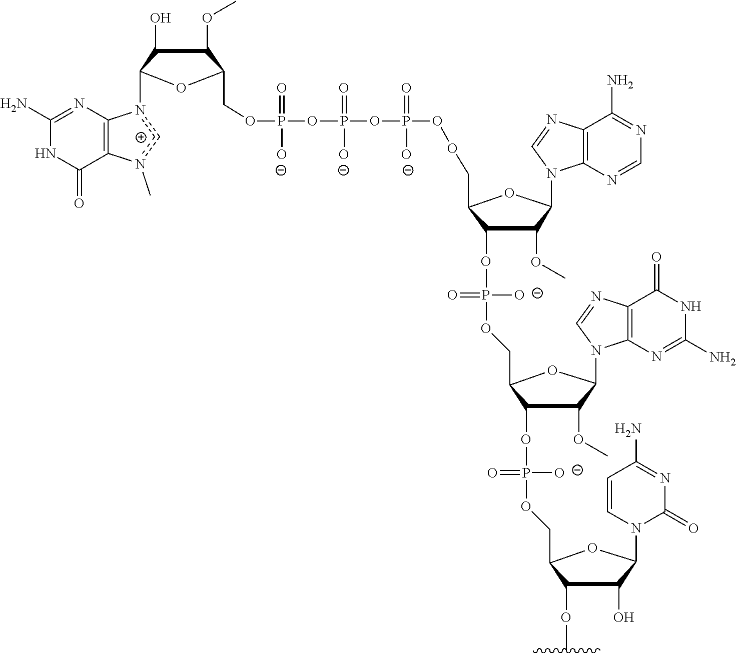

or a salt thereof.

In some embodiments, the 5′ cap is (m⁷)Gppp(m^{2′-O})Ap(m^{2′-O})UpA, having a structure:

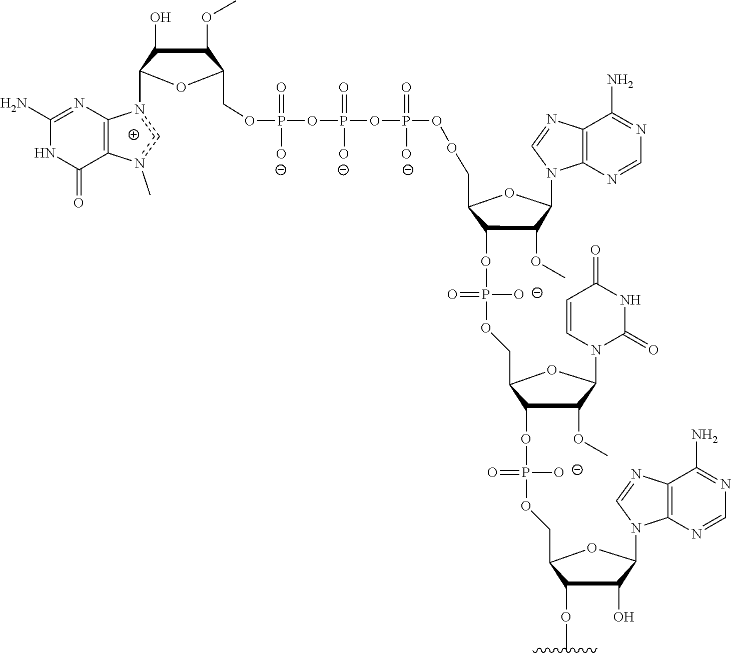

or a salt thereof.

In some embodiments, the 5′ cap is (m⁷)Gppp(m^{2′-O})Ap(m^{2′O})GpG, having a structure:

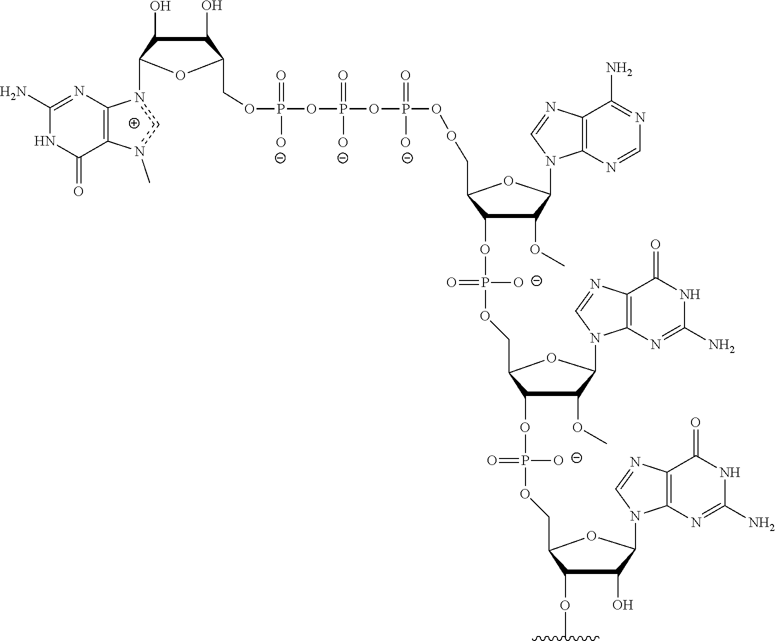

or a salt thereof.

### 2. Cap Proximal Sequences

In some embodiments, a 5′ UTR utilized in accordance with the present disclosure comprises a cap proximal sequence, e.g., as disclosed herein. In some embodiments, a cap proximal sequence comprises a sequence adjacent to a 5′ cap. In some embodiments, a cap proximal sequence comprises nucleotides in positions +1, +2, +3, +4, and/or +5 of an RNA polynucleotide.

In some embodiments, a cap structure comprises one or more polynucleotides of a cap proximal sequence. In some embodiments, a cap structure comprises an m⁷ Guanosine cap and nucleotide +1 (N₁) of an RNA polynucleotide. In some embodiments, a cap structure comprises an m⁷ Guanosine cap and nucleotide +2 (N₂) of an RNA polynucleotide. In some embodiments, a cap structure comprises an m⁷ Guanosine cap and nucleotides +1 and +2 (N₁ and N₂) of an RNA polynucleotide. In some embodiments, a cap structure comprises an m⁷ Guanosine cap and nucleotides +1, +2, and +3 (N₁, N₂, and N₃) of an RNA polynucleotide.

Those skilled in the art, reading the present disclosure, will appreciate that, in some embodiments, one or more residues of a cap proximal sequence (e.g., one or more of residues +1, +2, +3, +4, and/or +5) may be included in an RNA by virtue of having been included in a cap entity (e.g., a cap1 or cap2 structure, etc.); alternatively, in some embodiments, at least some of the residues in a cap proximal sequence may be enzymatically added (e.g., by a polymerase such as a T7 polymerase). For example, in certain exemplified embodiments where a m₂^{7,3-O}Gppp(m₁^{2-O})ApG cap is utilized, +1 (i.e., N₁) and +2 (i.e. N₂) are the (m₁^{2′-O})A and G residues of the cap, and +3, +4, and +5 are added by polymerase (e.g., T7 polymerase).

In some embodiments, the 5′ cap is a dinucleotide cap structure, wherein the cap proximal sequence comprises N₁ of the 5′ cap, where N₁ is any nucleotide, e.g., A, C, G or U. In some embodiments, the 5′ cap is a trinucleotide cap structure (e.g., the trinucleotide cap structures described above and herein), wherein the cap proximal sequence comprises N₁ and N₂ of the 5′ cap, wherein N₁ and N₂ are independently any nucleotide, e.g., A, C, G or U. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁, N₂, and N₃ are any nucleotide, e.g., A, C, G or U.

In some embodiments, e.g., where the 5′ cap is a dinucleotide cap structure, a cap proximal sequence comprises N₁ of a the 5′ cap, and N₂, N₃, N₄ and N₅, wherein N₁ to N₅ correspond to positions +1, +2, +3, +4, and/or +5 of an RNA polynucleotide. In some embodiments, e.g., where the 5′ cap is a trinucleotide cap structure, a cap proximal sequence comprises N₁ and N₂ of a the 5′ cap, and N₃, N₄ and N₅, wherein N₁ to N₅ correspond to positions +1, +2, +3, +4, and/or +5 of an RNA polynucleotide. In some embodiments, e.g., where the 5′ cap is a tetranucleotide cap structure, a cap proximal sequence comprises N₁, N₂, and N₃ of a the 5′ cap, and N₄ and N₅, wherein N₁ to N₅ correspond to positions +1, +2, +3, +4, and/or +5 of an RNA polynucleotide.

In some embodiments, N₁ is A. In some embodiments, N₁ is C. In some embodiments, N₁ is G. In some embodiments, N₁ is U. In some embodiments, N₂ is A. In some embodiments, N₂ is C. In some embodiments, N₂ is G. In some embodiments, N₂ is U. In some embodiments, N₃ is A. In some embodiments, N₃ is C. In some embodiments, N₃ is G. In some embodiments, N₃ is U. In some embodiments, N₄ is A. In some embodiments, N₄ is C. In some embodiments, N₄ is G. In some embodiments, N₄ is U. In some embodiments, N₅ is A. In some embodiments, N₅ is C. In some embodiments, N₅ is G. In some embodiments, N₅ is U. It will be understood that, each of the embodiments described above and herein (e.g., for N₁ through N₅) may be taken singly or in combination and/or may be combined with other embodiments of variables described above and herein (e.g., 5′ caps).

In some embodiments, a cap proximal sequence comprises A1 and G₂ of the Cap1 structure, and a sequence comprising: A₃A₄U₅ (SEQ ID NO: 150) at positions +3, +4 and +5 respectively of the polyribonucleotide.

### 3. 5′ UTR

In some embodiments, a nucleic acid (e.g., DNA, RNA) utilized in accordance with the present disclosure comprises a 5′-UTR. In some embodiments, 5′-UTR may comprise a plurality of distinct sequence elements; in some embodiments, such plurality may be or comprise multiple copies of one or more particular sequence elements (e.g., as may be from a particular source or otherwise known as a functional or characteristic sequence element). In some embodiments, a 5′ UTR comprises multiple different sequence elements.

The term “untranslated region” or “UTR” is commonly used in the art to a region in a DNA molecule which is transcribed but is not translated into an amino acid sequence, or to the corresponding region in an RNA polynucleotide, such as an RNA molecule. An untranslated region (UTR) can be present 5′ (upstream) of an open reading frame (5′-UTR) and/or 3′ (downstream) of an open reading frame (3′-UTR). As used herein, the terms “five prime untranslated region” or “5′ UTR” refer to a sequence of a polyribonucleotide between the 5′ end of the polyribonucleotide (e.g., a transcription start site) and a start codon of a coding region of the polyribonucleotide. In some embodiments, “5′ UTR” refers to a sequence of a polyribonucleotide that begins at the 5′ end of the polyribonucleotide (e.g., a transcription start site) and ends one nucleotide (nt) before a start codon (usually AUG) of a coding region of the polyribonucleotide, e.g., in its natural context. In some embodiments, a 5′ UTR comprises a Kozak sequence. A 5′-UTR is downstream of the 5′-cap (if present), e.g., directly adjacent to the 5′-cap. In some embodiments, a 5′ UTR disclosed herein comprises a cap proximal sequence, e.g., as defined and described herein. In some embodiments, a cap proximal sequence comprises a sequence adjacent to a 5′ cap.

Example 5′ UTRs include a human alpha globin (hAg) 5′UTR or a fragment thereof, a TEV 5′ UTR or a fragment thereof, a HSP70 5′ UTR or a fragment thereof, or a c-June 5′ UTR or a fragment thereof.

In some embodiments, an RNA disclosed herein comprises a hAg 5′ UTR or a fragment thereof.

In some embodiments, an RNA disclosed herein comprises a 5′ UTR having at least 80%, at least 85%, at least 90%, at least 95%, at least 96%, at least 97%, at least 98%, or at least 99% identity to a 5′ UTR with the sequence AGAATAAACTAGTATTCTTCTGGTCCCCACAGACTCAGAGAGAACCCGCCACC (SEQ ID NO: 151). In some embodiments, an RNA disclosed herein comprises a 5′ UTR having the sequence

(SEQ ID NO: 151)
AGAATAAACTAGTATTCTTCTGGTCCCCACAGACTCAGAGAGAAC
CCGCCACC.

In some embodiments, an RNA disclosed herein comprises a 5′ UTR having at least 80%, at least 85%, at least 90%, at least 95%, at least 96%, at least 97%, at least 98%, or at least 99% identity to a 5′ UTR with the sequence AACUAGUAUUCUUCUGGUCCCCACAGACUCAGAGAGAACCCGCCACC (SEQ ID NO: 152) (hAg-Kozak/5′UTR). In some embodiments, an RNA disclosed herein comprises a 5′ UTR having the sequence

(hAg-Kozak/5′UTR)
(SEQ ID NO: 152)
AACUAGUAUUCUUCUGGUCCCCACAGACUCAGAGAGAACCCGCCA
CC.

### 4. PolyA Tail

In some embodiments, a polynucleotide (e.g., DNA, RNA) disclosed herein comprises a polyadenylate (polyA) sequence, e.g., as described herein. In some embodiments, a polyA sequence is situated downstream of a 3′-UTR, e.g., adjacent to a 3′-UTR.

As used herein, the term “poly(A) sequence” or “poly-A tail” refers to an uninterrupted or interrupted sequence of adenylate residues which is typically located at the 3′-end of an RNA polynucleotide. Poly(A) sequences are known to those of skill in the art and may follow the 3′-UTR in the RNAs described herein. An uninterrupted poly(A) sequence is characterized by consecutive adenylate residues. In nature, an uninterrupted poly(A) sequence is typical. In some embodiments, polynucleotides disclosed herein comprise an uninterrupted Poly(A) sequence. In some embodiments, polynucleotides disclosed herein comprise interrupted Poly(A) sequence. In some embodiments, RNAs disclosed herein can have a poly(A) sequence attached to the free 3′-end of the RNA by a template-independent RNA polymerase after transcription or a poly(A) sequence encoded by DNA and transcribed by a template-dependent RNA polymerase.

It has been demonstrated that a poly(A) sequence of about 120 A nucleotides has a beneficial influence on the levels of RNA in transfected eukaryotic cells, as well as on the levels of protein that is translated from an open reading frame that is present upstream (5′) of the poly(A) sequence (Holtkamp et al., 2006, Blood, vol. 108, pp. 4009-4017, which is herein incorporated by reference).

In some embodiments, a poly(A) sequence in accordance with the present disclosure is not limited to a particular length; in some embodiments, a poly(A) sequence is any length. In some embodiments, a poly(A) sequence comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly(A) sisequence, typically at least 75%, at least 80%, at least 85%, at least 90%, at least 95%, at least 96%, at least 97%, at least 98%, or at least 99% by number of nucleotides in the poly(A) sisequence are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly(A) sisequence, i.e., 100% by number of nucleotides in the poly(A) sisequence, are A nucleotides. The term “A nucleotide” or “A” refers to adenylate.

In some embodiments, a poly(A) sequence is attached during RNA transcription, e.g., during preparation of in vitro transcribed RNA, based on a DNA template comprising repeated dT nucleotides (deoxythymidylate) in the strand complementary to the coding strand. The DNA sequence encoding a poly(A) sequence (coding strand) is referred to as poly(A) cassette.

In some embodiments, the poly(A) sicassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A₁, hereby incorporated by reference. Any poly(A) cassette disclosed in WO 2016/005324 A₁, which is incorporated herein by reference in its entirety, may be used in accordance with the present disclosure. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. In some embodiments, the poly(A) sequence contained in an RNA polynucleotide described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, no nucleotides other than A nucleotides flank a poly(A) sequence at its 3′-end, i.e., the poly(A) sisequence is not masked or followed at its 3′-end by a nucleotide other than A.

In some embodiments, the poly(A) sisequence may comprise at least 20, at least 30, at least 40, at least 80, or at least 100 and up to 500, up to 400, up to 300, up to 200, or up to 150 nucleotides. In some embodiments, the poly(A) sisequence may essentially consist of at least 20, at least 30, at least 40, at least 80, or at least 100 and up to 500, up to 400, up to 300, up to 200, or up to 150 nucleotides. In some embodiments, the poly(A) sisequence may consist of at least 20, at least 30, at least 40, at least 80, or at least 100 and up to 500, up to 400, up to 300, up to 200, or up to 150 nucleotides. In some embodiments, the poly(A) sisequence comprises at least 100 nucleotides. In some embodiments, the poly(A) sisequence comprises about 150 nucleotides. In some embodiments, the poly(A) sisequence comprises about 120 nucleotides.

In some embodiments, a poly A tail comprises a specific number of Adenosines, such as about 50 or more, about 60 or more, about 70 or more, about 80 or more, about 90 or more, about 100 or more, about 120, or about 150 or about 200. In some embodiments a poly A tail of a string construct may comprise 200 A residues or less. In some embodiments, a poly A tail of a string construct may comprise about 200 A residues. In some embodiments, a poly A tail of a string construct may comprise 180 A residues or less. In some embodiments, a poly A tail of a string construct may comprise about 180 A residues. In some embodiments, a poly A tail may comprise 150 residues or less.

In some embodiments, RNA comprises a poly(A) sequence comprising the nucleotide sequence of AAAAAAAAAAAAAAAAAAAAAAAAAAAAAAGCATATGACTAAAAAAAAAAAAAA AAAAAAAAAAAAAAAAAAAAAAAAAAAAAAAAAAAAAAAAAAAAAAAAAAAAAA AA (SEQ ID NO: 153), or a nucleotide sequence having at least 99%, 98%, 97%, 96%, 95%, 90%, 85%, or 80% identity to the nucleotide sequence of AAAAAAAAAAAAAAAAAAAAAAAAAAAAAAGCATATGACTAAAAAAAAAAAAAA AAAAAAAAAAAAAAAAAAAAAAAAAAAAAAAAAAAAAAAAAAAAAAAAAAAAAA AA (SEQ ID NO: 153). In some embodiments, a poly(A) tail comprises a plurality of A residues interrupted by a linker. In some embodiments, a linker comprises the nucleotide sequence GCATATGAC (SEQ ID NO: 154).

In some embodiments, RNA comprises a poly(A) sequence comprising the nucleotide sequence of AAAAAAAAAAAAAAAAAAAAAAAAAAAAAAGCAUAUGACUAAAAAAAAAAAAAA AAAAAAAAAAAAAAAAAAAAAAAAAAAAAAAAAAAAAAAAAAAAAAAAAAAAAA AA (SEQ ID NO: 155), or a nucleotide sequence having at least 99%, 98%, 97%, 96%, 95%, 90%, 85%, or 80% identity to the nucleotide sequence of AAAAAAAAAAAAAAAAAAAAAAAAAAAAAAGCAUAUGACUAAAAAAAAAAAAAA AAAAAAAAAAAAAAAAAAAAAAAAAAAAAAAAAAAAAAAAAAAAAAAAAAAAAA AA (SEQ ID NO: 155). In some embodiments, a poly(A) tail comprises a plurality of A residues interrupted by a linker. In some embodiments, a linker comprises the nucleotide sequence GCAUAUGAC (SEQ ID NO: 156).

### 5. 3′ UTR

In some embodiments, an RNA utilized in accordance with the present disclosure comprises a 3′-UTR. As used herein, the terms “three prime untranslated region,” “3′ untranslated region,” or “3′ UTR” refer to a sequence of an RNA molecule that begins following a stop codon of a coding region of an open reading frame sequence. In some embodiments, the 3′ UTR begins immediately after a stop codon of a coding region of an open reading frame sequence, e.g., in its natural context. In other embodiments, the 3′ UTR does not begin immediately after stop codon of the coding region of an open reading frame sequence, e.g., in its natural context. The term “3′-UTR” does preferably not include the poly(A) sisequence. Thus, the 3′-UTR is upstream of the poly(A) sisequence (if present), e.g. directly adjacent to the poly(A) sequence.

In some embodiments, an RNA disclosed herein comprises a 3′ UTR comprising an F element and/or an I element. In some embodiments, a 3′ UTR or a proximal sequence thereto comprises a restriction site. In some embodiments, a restriction site is a BamHI site. In some embodiments, a restriction site is a XhoI site.

In some embodiments, an RNA construct comprises an F element. In some embodiments, a F element sequence is a 3′-UTR of amino-terminal enhancer of split (AES).

In some embodiments, an RNA disclosed herein comprises a 3′ UTR having at least 80%, at least 85%, at least 90%, at least 95%, at least 96%, at least 97%, at least 98%, or at least 99% identity to a 3′ UTR with the sequence of CTGGTACTGCATGCACGCAATGCTAGCTGCCCCTTTCCCGTCCTGGGTACCCCGAGT CTCCCCCGACCTCGGGTCCCAGGTATGCTCCCACCTCCACCTGCCCCACTCACCACC TCTGCTAGTTCCAGACACCTCCCAAGCACGCAGCAATGCAGCTCAAAACGCTTAGCC TAGCCACACCCCCACGGGAAACAGCAGTGATTAACCTTTAGCAATAAACGAAAGTT TAACTAAGCTATACTAACCCCAGGGTTGGTCAATTTCGTGCCAGCCACACC (SEQ ID NO: 157). In some embodiments, an RNA disclosed herein comprises a 3′ UTR with the sequence of

(SEQ ID NO: 157)
CTGGTACTGCATGCACGCAATGCTAGCTGCCCCTTTCCCGTCCTG
GGTACCCCGAGTCTCCCCCGACCTCGGGTCCCAGGTATGCTCCCA
CCTCCACCTGCCCCACTCACCACCTCTGCTAGTTCCAGACACCTC
CCAAGCACGCAGCAATGCAGCTCAAAACGCTTAGCCTAGCCACAC
CCCCACGGGAAACAGCAGTGATTAACCTTTAGCAATAAACGAAAG
TTTAACTAAGCTATACTAACCCCAGGGTTGGTCAATTTCGTGCCA
GCCACACC.

In some embodiments, an RNA disclosed herein comprises a 3′ UTR having at least 80%, at least 85%, at least 90%, at least 95%, at least 96%, at least 97%, at least 98%, or at least 99% identity to a 3′ UTR with the sequence of CUGGUACUGCAUGCACGCAAUGCUAGCUGCCCCUUUCCCGUCCUGGGUACCCCGA GUCUCCCCCGACCUCGGGUCCCAGGUAUGCUCCCACCUCCACCUGCCCCACUCACC ACCUCUGCUAGUUCCAGACACCUCCCAAGCACGCAGCAAUGCAGCUCAAAACGCU UAGCCUAGCCACACCCCCACGGGAAACAGCAGUGAUUAACCUUUAGCAAUAAACG AAAGUUUAACUAAGCUAUACUAACCCCAGGGUUGGUCAAUUUCGUGCCAGCCACA CC (SEQ ID NO: 158). In some embodiments, an RNA disclosed herein comprises a 3′ UTR with the sequence of

(SEQ ID NO: 158)
CUGGUACUGCAUGCACGCAAUGCUAGCUGCCCCUUUCCCGUCCUG
GGUACCCCGAGUCUCCCCCGACCUCGGGUCCCAGGUAUGCUCCCA
CCUCCACCUGCCCCACUCACCACCUCUGCUAGUUCCAGACACCUC
CCAAGCACGCAGCAAUGCAGCUCAAAACGCUUAGCCUAGCCACAC
CCCCACGGGAAACAGCAGUGAUUAACCUUUAGCAAUAAACGAAAG
UUUAACUAAGCUAUACUAACCCCAGGGUUGGUCAAUUUCGUGCCA
GCCACACC.

In some embodiments, a 3′UTR is an FI element as described in WO2017/060314, which is herein incorporated by reference in its entirety.

### B. RNA Formats

At least three distinct formats useful for RNA compositions (e.g., pharmaceutical compositions) have been developed, namely non-modified uridine containing RNA (uRNA), nucleoside-modified RNA (modRNA), and self-amplifying RNA (saRNA). Each of these platforms displays unique features. In general, in all three formats, RNA is capped, contains open reading frames (ORFs) flanked by untranslated regions (UTR), and have a polyA-tail at the 3′ end. An ORF of an uRNA and modRNA vectors encode an antibody agent or fragment thereof. An saRNA has multiple ORFs.

In some embodiments, the RNA described herein may have modified nucleosides. In some embodiments, the RNA comprises a modified nucleoside in place of at least one (e.g., every) uridine.

The term “uracil,” as used herein, describes one of the nucleobases that can occur in the nucleic acid of RNA. The structure of uracil is:

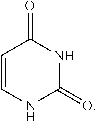

The term “uridine,” as used herein, describes one of the nucleosides that can occur in RNA. The structure of uridine is:

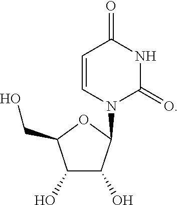 [SMILES: O=c1ccn([C@@H]2O[C@H](CO)[C@@H](O)[C@H]2O)c(=O)[nH]1]

UTP (uridine 5′-triphosphate) has the following structure:

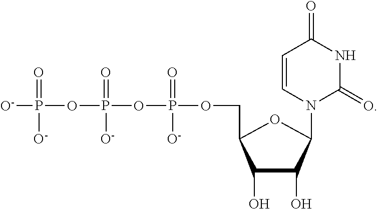

Pseudo-UTP (pseudouridine 5′-triphosphate) has the following structure:

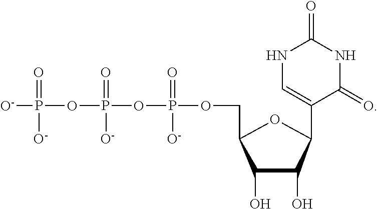

“Pseudouridine” is one example of a modified nucleoside that is an isomer of uridine, where the uracil is attached to the pentose ring via a carbon-carbon bond instead of a nitrogen-carbon glycosidic bond.

Another exemplary modified nucleoside is N1-methyl-pseudouridine (m1Ψ), which has the structure:

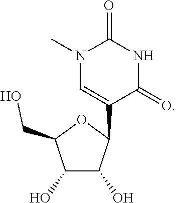 [SMILES: Cn1cc([C@@H]2O[C@H](CO)[C@@H](O)[C@H]2O)c(=O)[nH]c1=O]

N1-methyl-pseudo-UTP has the following structure:

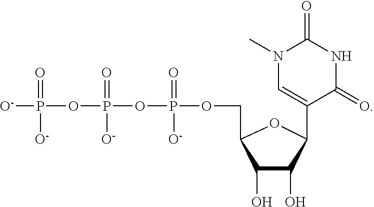

Another exemplary modified nucleoside is 5-methyl-uridine (m5U), which has the structure:

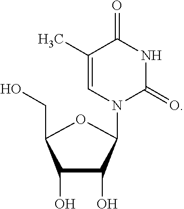

In some embodiments, one or more uridine in an RNA described herein is replaced by a modified nucleoside. In some embodiments, the modified nucleoside is a modified uridine.

In some embodiments, RNA comprises a modified nucleoside in place of at least one uridine. In some embodiments, RNA comprises a modified nucleoside in place of each uridine.

In some embodiments, the modified nucleoside is independently selected from pseudouridine (ψ), N1-methyl-pseudouridine (m1ψ), and 5-methyl-uridine (m5U). In some embodiments, the modified nucleoside comprises pseudouridine (ψ). In some embodiments, the modified nucleoside comprises N1-methyl-pseudouridine (m1ψ). In some embodiments, the modified nucleoside comprises 5-methyl-uridine (m5U). In some embodiments, RNA may comprise more than one type of modified nucleoside, and the modified nucleosides are independently selected from pseudouridine (ψ), N1-methyl-pseudouridine (m1ψ), and 5-methyl-uridine (m5U). In some embodiments, the modified nucleosides comprise pseudouridine (ψ) and N1-methyl-pseudouridine (m1ψ). In some embodiments, the modified nucleosides comprise pseudouridine (ψ) and 5-methyl-uridine (m5U). In some embodiments, the modified nucleosides comprise N1-methyl-pseudouridine (m1ψ) and 5-methyl-uridine (m5U). In some embodiments, the modified nucleosides comprise pseudouridine (ψ), N1-methyl-pseudouridine (m1ψ), and 5-methyl-uridine (m5U).

In some embodiments, the modified nucleoside replacing one or more, e.g., all, uridine in the RNA may be any one or more of 3-methyl-uridine (m3U), 5-methoxy-uridine (mo5U), 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridine or 5-bromo-uridine), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 1-ethyl-pseudouridine, 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (tm5U), 1-taurinomethyl-pseudouridine, 5-taurinomethyl-2-thio-uridine (m5s2U), 1-taurinomethyl-4-thio-pseudouridine), 5-methyl-2-thio-uridine (m5s2U), 1-methyl-4-thio-pseudouridine (mls4ψ), 4-thio-1-methyl-pseudouridine, 3-methyl-pseudouridine (m3v), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine (acp3U), 1-methyl-3-(3-amino-3-carboxypropyl) pseudouridine (acp3 w), 5-(isopentenylaminomethyl) uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), 5-(isopentenylaminomethyl)-2′-O-methyl-uridine (inm5Um), 1-thio-uridine, deoxythymidine, 2′-F-ara-uridine, 2′-F-uridine, 2′—OH-ara-uridine, 5-(2-carbomethoxyvinyl) uridine, 5-[3-(1-E-propenylamino) uridine, or any other modified uridine known in the art.

In some embodiments, the RNA comprises other modified nucleosides or comprises further modified nucleosides, e.g., modified cytidine. For example, in some embodiments, in the RNA 5-methylcytidine is substituted partially or completely, preferably completely, for cytidine. In some embodiments, the RNA comprises 5-methylcytidine and one or more selected from pseudouridine (ψ), N1-methyl-pseudouridine (m1ψ), and 5-methyl-uridine (m5U). In some embodiments, the RNA comprises 5-methylcytidine and N1-methyl-pseudouridine (m1ψ). In some embodiments, the RNA comprises 5-methylcytidine in place of each cytidine and N1-methyl-pseudouridine (m1ψ) in place of each uridine.

In some embodiments of the present disclosure, the RNA is “replicon RNA” or simply a “replicon,” in particular “self-replicating RNA” or “self-amplifying RNA.” In one particularly preferred embodiment, the replicon or self-replicating RNA is derived from or comprises elements derived from a single-stranded (ss) RNA virus, in particular a positive-stranded ssRNA virus, such as an alphavirus. Alphaviruses are typical representatives of positive-stranded RNA viruses. Alphaviruses replicate in the cytoplasm of infected cells (for review of the alphaviral life cycle see José et al., Future Microbiol., 2009, vol. 4, pp. 837-856, which is incorporated herein by reference in its entirety). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res., vol. 87 pp. 111-124, which is incorporated herein by reference in its entirety). Following infection, i.e., at early stages of the viral life cycle, the (+) stranded genomic RNA directly acts like a messenger RNA for the translation of the open reading frame encoding the non-structural poly-protein (nsP1234).

Alphavirus-derived vectors have been proposed for delivery of foreign genetic information into target cells or target organisms. In simple approaches, a first ORF encodes an alphavirus-derived RNA-dependent RNA polymerase (replicase), which upon translation mediates self-amplification of the RNA. A second ORF encoding alphaviral structural proteins is replaced by an open reading frame encoding an HSV-2 construct described herein. Alphavirus-based trans-replication systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

Features of a non-modified uridine platform may include, for example, one or more of intrinsic adjuvant effect, as well as good tolerability and safety. Features of modified uridine (e.g., pseudouridine) platform may include reduced adjuvant effect, blunted immune innate immune sensor activating capacity and thus good tolerability and safety. Features of self-amplifying platform may include, for example, long duration of protein expression, good tolerability and safety, higher likelihood for efficacy with very low vaccine dose.

The present disclosure provides particular RNA constructs optimized, for example, for improved manufacturability, encapsulation, expression level (and/or timing), etc. Certain components are discussed below, and certain preferred embodiments are exemplified herein.

### C. Codon Optimization and GC Enrichment

As used herein, the term “codon-optimized” refers to alteration of codons in a coding region of a nucleic acid molecule (e.g., a polyribonucleotide) to reflect the typical codon usage of a host organism (e.g., a subject receiving a nucleic acid molecule (e.g., a polyribonucleotide)) without preferably altering the amino acid sequence encoded by the nucleic acid molecule. Within the context of the present disclosure, in some embodiments, coding regions are codon-optimized for optimal expression in a subject to be treated using the RNA molecules described herein. In some embodiments, codon-optimization may be performed such that codons for which frequently occurring tRNAs are available are inserted in place of “rare codons.” In some embodiments, codon-optimization may include increasing guanosine/cytosine (G/C) content of a coding region of RNA described herein as compared to the G/C content of the corresponding coding sequence of a wild type RNA, wherein the amino acid sequence encoded by the RNA is preferably not modified compared to the amino acid sequence.

In some embodiments, a coding sequence (also referred to as a “coding region”) is codon optimized for expression in the subject to whom a composition (e.g., a pharmaceutical composition) is to be administered (e.g., a human). Thus, in some embodiments, sequences in such a polynucleotide (e.g., a polyribonucleotide) may differ from wild type sequences encoding the relevant antigen or fragment or epitope thereof, even when the amino acid sequence of the antigen or fragment or epitope thereof is wild type.

In some embodiments, strategies for codon optimization for expression in a relevant subject (e.g., a human), and even, in some cases, for expression in a particular cell or tissue.

Various species exhibit particular bias for certain codons of a particular amino acid. Without wishing to be bound by any one theory, codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell may generally be a reflection of the codons used most frequently in peptide synthesis. Accordingly, genes may be tailored for optimal gene expression in a given organism based on codon optimization. Codon usage tables are available, for example, at the “Codon Usage Database” available at www.kazusa.orjp/codon/and these tables may be adapted in a number of ways. Computer algorithms for codon optimizing a particular sequence for expression in a particular subject or its cells are also available, such as Gene Forge (Aptagen; Jacobus, PA), are also available.

In some embodiments, a polynucleotide (e.g., a polyribonucleotide) of the present disclosure is codon optimized, wherein the codons in the polynucleotide (e.g., the polyribonucleotide) are adapted to human codon usage (herein referred to as “human codon optimized polynucleotide”). In some embodiments, a portion of a polyribonucleotide is codon optimized (e.g., a portion of or the portion encoding a glycoprotein or a portion of or the portion encoding a secretory signal). In some embodiments, the entire polyribonucleotide is codon optimized. Codons encoding the same amino acid occur at different frequencies in a subject, e.g., a human. Accordingly, in some embodiments, the coding sequence of a polynucleotide of the present disclosure is modified such that the frequency of the codons encoding the same amino acid corresponds to the naturally occurring frequency of that codon according to the human codon usage, e.g., as shown in Table 14. For example, in the case of the amino acid Ala, the wild type coding sequence is preferably adapted in a way that the codon “GCC” is used with a frequency of 0.40, the codon “GCT” is used with a frequency of 0.28, the codon “GCA” is used with a frequency of 0.22 and the codon “GCG” is used with 30 a frequency of 0.10 etc. (see Table 14). Accordingly, in some embodiments, such a procedure (as exemplified for Ala) is applied for each amino acid encoded by the coding sequence of a polynucleotide to obtain sequences adapted to human codon usage.

TABLE 14
Human codon usage with frequencies
indicated for each amino acid.

| Amino | | | Amino | | |
|---|---|---|---|---|---|
| acid | codon | frequency | acid | codon | frequency |
| Ala | GCG | 0.10 | Pro | CCG | 0.11 |
| Ala | GCA | 0.22 | Pro | CCA | 0.27 |
| Ala | GCT | 0.28 | Pro | CCT | 0.29 |
| Ala | GCC* | 0.40 | Pro | CCC* | 0.33 |
| Cys | TGT | 0.42 | Gin | CAG* | 0.73 |
| Cys | TGC* | 0.58 | Gin | CAA | 0.27 |
| Asp | GAT | 0.44 | Arg | AGG | 0.22 |
| Asp | GAC* | 0.56 | Arg | AGA* | 0.21 |
| Glu | GAG* | 0.59 | Arg | CGG | 0.19 |
| Glu | GAA | 0.41 | Arg | CGA | 0.10 |
| Phe | TTT | 0.43 | Arg | CGT | 0.09 |
| Phe | TTC* | 0.57 | Arg | CGC | 0.19 |
| Gly | GGG | 0.23 | Ser | AGT | 0.14 |
| Gly | GGA | 0.26 | Ser | AGC* | 0.25 |
| Gly | GGT | 0.18 | Ser | TCG | 0.06 |
| Gly | GGC* | 0.33 | Ser | TCA | 0.15 |
| His | CAT | 0.41 | Ser | TCT | 0.18 |
| His | CAC* | 0.59 | Ser | TCC | 0.23 |
| lle | ATA | 0.14 | Thr | ACG | 0.12 |
| lle | ATT | 0.35 | Thr | ACA | 0.27 |
| lle | ATC* | 0.52 | Thr | ACT | 0.23 |
| Lys | AAG* | 0.60 | Tor | ACC* | 0.38 |
| Lys | AAA | 0.40 | Val | GTG* | 0.48 |
| Leu | TTG | 0.12 | Val | GTA | 0.10 |
| Leu | TTA | 0.06 | Val | GTT | 0.17 |
| Leu | CTG* | 0.43 | Val | GTC | 0.25 |
| Leu | CTA | 0.07 | Trp | TGG* | 1 |
| Leu | CTT | 0.12 | Tyr | TAT | 0 42 |
| Lou | CTC | 0.20 | Tyr | TAC* | 0.58 |
| Met | ATG* | 1 | Stop | TGA* | 0 61 |
| Asn | AAT | 0.44 | Stop | TAG | 0.17 |
| Asn | AAC* | 0.56 | Stop | TAA | 0.22 |

Certain strategies for codon optimization and/or G/C enrichment for human expression are described in WO2002/098443, which is incorporated by reference herein in its entirety. In some embodiments, a coding sequence may be optimized using a multiparametric optimization strategy. In some embodiments, optimization parameters may include parameters that influence protein expression, which can be, for example, impacted on a transcription level, an RNA level, and/or a translational level. In some embodiments, exemplary optimization parameters include, but are not limited to transcription-level parameters (including, e.g., GC content, consensus splice sites, cryptic splice sites, SD sequences, TATA boxes, termination signals, artificial recombination sites, and combinations thereof); RNA-level parameters (including, e.g., RNA instability motifs, ribosomal entry sites, repetitive sequences, and combinations thereof); translation-level parameters (including, e.g., codon usage, premature poly(A) sites, ribosomal entry sites, secondary structures, and combinations thereof); or combinations thereof. In some embodiments, a coding sequence may be optimized by a GeneOptimizer algorithm as described in Fath et al. “Multiparameter RNA and Codon Optimization: A Standardized Tool to Assess and Enhance Autologous Mammalian Gene Expression” PLOS ONE 6 (3): e17596; Rabb et al., which is incorporated herein by reference in its entirety, “The GeneOptimizer Algorithm: using a sliding window approach to cope with the vast sequence space in multiparameter DNA sequence optimization” Systems and Synthetic Biology (2010) 4:215-225; and Graft et al. “Codon-optimized genes that enable increased heterologous expression in mammalian cells and elicit efficient immune responses in mice after vaccination of naked DNA” Methods Mol Med (2004) 94:197-210, the entire content of each of which is incorporated herein for the purposes described herein. In some embodiments, a coding sequence may be optimized by Eurofins' adaption and optimization algorithm “GENEius” as described in Eurofins' Application Notes: Eurofins' adaption and optimization software “GENEius” in comparison to other optimization algorithms, the entire content of which is incorporated by reference for the purposes described herein.

In some embodiments, a coding sequence utilized in accordance with the present disclosure has G/C content that is increased compared to a coding sequence for an HSV gC, gD, and/or gE (or fragment thereof) construct described herein. In some embodiments, guanosine/cytidine (G/C) content of a coding region is modified relative to a comparable coding sequence for an HSV gC, gD, and/or gE (or fragment thereof) construct described herein, but the amino acid sequence encoded by the polyribonucleotide not modified.

Without wishing to be bound by any particular theory, it is proposed that GC enrichment may improve translation of a payload sequence. Typically, sequences having an increased G (guanosine)/C (cytidine) content are more stable than sequences having an increased A (adenosine)/U (uridine) content. In respect to the fact that several codons code for one and the same amino acid (so-called degeneration of the genetic code), the most favorable codons for the stability can be determined (so-called alternative codon usage). Depending on the amino acid to be encoded by a polyribonucleotide, there are various possibilities for modification of the ribonucleic acid sequence, compared to its wild type sequence. In particular, codons which contain A and/or U nucleosides can be modified by substituting these codons by other codons, which code for the same amino acids but contain no A and/or U or contain a lower content of A and/or U nucleosides.

In some embodiments, G/C content of a coding region of a polyribonucleotide described herein is increased by at least 1%, at least 2%, at least 3%, at least 4%, at least 5%, at least 6%, or even more compared to the G/C content of the coding region prior to codon optimization, e.g., of the wild type RNA. In some embodiments, G/C content of a coding region of a polyribonucleotide described herein is decreased by at least 1%, at least 2%, at least 3%, at least 4%, at least 5%, at least 6%, or even more compared to the G/C content of the coding region prior to codon optimization, e.g., of the wild type RNA.

In some embodiments, stability and translation efficiency of a polyribonucleotide may incorporate one or more elements established to contribute to stability and/or translation efficiency of the polyribonucleotide; exemplary such elements are described, for example, in PCT/EP2006/009448 incorporated herein by reference. In some embodiments, to increase expression of a polyribonucleotide used according to the present disclosure, a polyribonucleotide may be modified within the coding region, i.e., the sequence encoding the expressed peptide or protein, without altering the sequence of the expressed peptide or protein, for example so as to increase the GC-content to increase RNA stability and/or to perform a codon optimization and, thus, enhance translation in cells.

### D. Certain Example RNA Constructs

In some embodiments, a polyribonucleotide provided herein encodes a polypeptide, wherein the polypeptide comprises an amino acid sequence according to SEQ ID NO: 65. In some embodiments, an RNA construct comprises a 5′ cap, a 5′UTR, a polyribonucleotide that encodes a polypeptide, wherein the polypeptide comprises an amino acid sequence according to SEQ ID NO: 65, a 3′ UTR, and a polyA tail.

In some embodiments, a polyribonucleotide provided herein encodes a polypeptide, wherein the polypeptide comprises an amino acid sequence according to SEQ ID NO: 70. In some embodiments, an RNA construct comprises a 5′ cap, a 5′UTR, a polyribonucleotide that encodes a polypeptide, wherein the polypeptide comprises an amino acid sequence according to SEQ ID NO: 70, a 3′ UTR, and a polyA tail.

In some embodiments, a polyribonucleotide provided herein encodes a polypeptide, wherein the polypeptide comprises an amino acid sequence according to SEQ ID NO: 73. In some embodiments, an RNA construct comprises a 5′ cap, a 5′UTR, a polyribonucleotide that encodes a polypeptide, wherein the polypeptide comprises an amino acid sequence according to SEQ ID NO: 73, a 3′ UTR, and a polyA tail.

In some embodiments, a polyribonucleotide provided herein encodes a polypeptide, wherein the polypeptide comprises an amino acid sequence according to SEQ ID NO: 67. In some embodiments, an RNA construct comprises a 5′ cap, a 5′UTR, a polyribonucleotide that encodes a polypeptide, wherein the polypeptide comprises an amino acid sequence according to SEQ ID NO: 67, a 3′ UTR, and a polyA tail.

In some embodiments, a polyribonucleotide provided herein encodes a polypeptide, wherein the polypeptide comprises an amino acid sequence according to SEQ ID NO: 68. In some embodiments, an RNA construct comprises a 5′ cap, a 5′UTR, a polyribonucleotide that encodes a polypeptide, wherein the polypeptide comprises an amino acid sequence according to SEQ ID NO: 68, a 3′ UTR, and a polyA tail.

In some embodiments, a polyribonucleotide provided herein encodes a polypeptide, wherein the polypeptide comprises an amino acid sequence according to SEQ ID NO: 75. In some embodiments, an RNA construct comprises a 5′ cap, a 5′UTR, a polyribonucleotide that encodes a polypeptide, wherein the polypeptide comprises an amino acid sequence according to SEQ ID NO: 75, a 3′ UTR, and a poly A tail.

In some embodiments, a polyribonucleotide provided herein encodes a polypeptide, wherein the polypeptide comprises an amino acid sequence according to SEQ ID NO: 131. In some embodiments, an RNA construct comprises a 5′ cap, a 5′UTR, a polyribonucleotide that encodes a polypeptide, wherein the polypeptide comprises an amino acid sequence according to SEQ ID NO: 131, a 3′ UTR, and a polyA tail.

In some embodiments, a polyribonucleotide provided herein encodes a polypeptide, wherein the polypeptide comprises an amino acid sequence according to SEQ ID NO: 132. In some embodiments, an RNA construct comprises a 5′ cap, a 5′UTR, a polyribonucleotide that encodes a polypeptide, wherein the polypeptide comprises an amino acid sequence according to SEQ ID NO: 132, a 3′ UTR, and a polyA tail.

In some embodiments, a polyribonucleotide provided herein encodes a polypeptide, wherein the polypeptide comprises an amino acid sequence according to SEQ ID NO: 159. In some embodiments, an RNA construct comprises a 5′ cap, a 5′UTR, a polyribonucleotide that encodes a polypeptide, wherein the polypeptide comprises an amino acid sequence according to SEQ ID NO: 159, a 3′ UTR, and a polyA tail.

In some embodiments, a polyribonucleotide provided herein encodes a polypeptide, wherein the polypeptide comprises an amino acid sequence according to SEQ ID NO: 160. In some embodiments, an RNA construct comprises a 5′ cap, a 5′UTR, a polyribonucleotide that encodes a polypeptide, wherein the polypeptide comprises an amino acid sequence according to SEQ ID NO: 160, a 3′ UTR, and a polyA tail.

In some embodiments, a polyribonucleotide provided herein encodes a polypeptide, wherein the polypeptide comprises an amino acid sequence according to SEQ ID NO: 161. In some embodiments, an RNA construct comprises a 5′ cap, a 5′UTR, a polyribonucleotide that encodes a polypeptide, wherein the polypeptide comprises an amino acid sequence according to SEQ ID NO: 161, a 3′ UTR, and a polyA tail.

In some embodiments, a polyribonucleotide provided herein encodes a polypeptide, wherein the polypeptide comprises an amino acid sequence according to SEQ ID NO: 162. In some embodiments, an RNA construct comprises a 5′ cap, a 5′UTR, a polyribonucleotide that encodes a polypeptide, wherein the polypeptide comprises an amino acid sequence according to SEQ ID NO: 162, a 3′ UTR, and a polyA tail.

In some embodiments, a polyribonucleotide provided herein encodes a polypeptide, wherein the polypeptide comprises an amino acid sequence according to SEQ ID NO: 163. In some embodiments, an RNA construct comprises a 5′ cap, a 5′UTR, a polyribonucleotide that encodes a polypeptide, wherein the polypeptide comprises an amino acid sequence according to SEQ ID NO: 163, a 3′ UTR, and a polyA tail.

In some embodiments, a polyribonucleotide provided herein encodes a polypeptide, wherein the polypeptide comprises an amino acid sequence according to SEQ ID NO: 164. In some embodiments, an RNA construct comprises a 5′ cap, a 5′UTR, a polyribonucleotide that encodes a polypeptide, wherein the polypeptide comprises an amino acid sequence according to SEQ ID NO: 164, a 3′ UTR, and a polyA tail.

In some embodiments, a polyribonucleotide provided herein encodes a polypeptide, wherein the polypeptide comprises an amino acid sequence according to SEQ ID NO: 327. In some embodiments, an RNA construct comprises a 5′ cap, a 5′UTR, a polyribonucleotide that encodes a polypeptide, wherein the polypeptide comprises an amino acid sequence according to SEQ ID NO: 327, a 3′ UTR, and a polyA tail.

In some embodiments, a polyribonucleotide provided herein encodes a polypeptide, wherein the polypeptide comprises an amino acid sequence according to SEQ ID NO: 328. In some embodiments, an RNA construct comprises a 5′ cap, a 5′UTR, a polyribonucleotide that encodes a polypeptide, wherein the polypeptide comprises an amino acid sequence according to SEQ ID NO: 328, a 3′ UTR, and a polyA tail.

### IV. RNA Delivery Technologies

Provided polyribonucleotides may be delivered for therapeutic applications described herein using any appropriate methods known in the art, including, e.g., delivery as naked RNAs, or delivery mediated by viral and/or non-viral vectors, polymer-based vectors, lipid compositions, nanoparticles (e.g., lipid nanoparticles, polymeric nanoparticles, lipid-polymer hybrid nanoparticles, etc.), and/or peptide-based vectors. See, e.g., Wadhwa et al. “Opportunities and Challenges in the Delivery of mRNA-Based Vaccines” Pharmaceutics (2020) 102 (27 pages), the content of which is incorporated herein by reference, for information on various approaches that may be useful for delivery polyribonucleotides described herein.

In some embodiments, one or more polyribonucleotides can be formulated with lipid nanoparticles for delivery (e.g., administration).

In some embodiments, lipid nanoparticles can be designed to protect polyribonucleotides from extracellular RNases and/or engineered for systemic delivery of the RNA to target cells. In some embodiments, such lipid nanoparticles may be particularly useful to deliver polyribonucleotides when polyribonucleotides are intravenously or intramuscularly administered to a subject.

### A. Lipid Compositions

### 1. Lipids and Lipid-Like Materials

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually poorly soluble in water. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of a polar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

A “lipid-like material” is a substance that is structurally and/or functionally related to a lipid but may not be considered a lipid in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids.

Specific examples of amphiphilic compounds that may be included in an amphiphilic layer include, but are not limited to, phospholipids, aminolipids and sphingolipids.

Generally, lipids may be divided into eight categories: fatty acids, glycerolipids, glycerophospholipids, sphingolipids, saccharolipids, polyketides (derived from condensation of ketoacyl subunits), sterols and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as sterol-containing metabolites such as cholesterol.

Fatty acids are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

Glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are members of a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterols, such as cholesterol and its derivatives, or tocopherol and its derivatives, are important components of membrane lipids, along with the glycerophospholipids and sphingomyelins.

Saccharolipids are compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

Lipids and lipid-like materials may be cationic, anionic or neutral. Neutral lipids or lipid-like materials exist in an uncharged or neutral zwitterionic form at a selected pH.

In some embodiments, suitable lipids or lipid-like materials for use in the present disclosure include those described in WO2020/128031 and US20200163878, the entire contents of each of which are incorporated herein by reference for the purposes described herein.

### 2. Cationic or Cationically Ionizable Lipids or Lipid-Like Materials

In some embodiments cationic or cationically ionizable lipids or lipid-like materials contemplated for use herein include any cationic or cationically ionizable lipids or lipid-like materials which are able to electrostatically bind nucleic acid. In one embodiment, cationic or cationically ionizable lipids or lipid-like materials contemplated for use herein can be associated with nucleic acid, e.g., by forming complexes with the nucleic acid or forming vesicles in which the nucleic acid is enclosed or encapsulated.

Cationic lipids or lipid-like materials are characterized in that they have a net positive charge (e.g., at a relevant pH). Cationic lipids or lipid-like materials bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge.

In certain embodiments, a cationic lipid or lipid-like material has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological pH.

In some embodiments, a cationic or cationically ionizable lipid or lipid-like material comprises a head group which includes at least one nitrogen atom (N) which is positive charged or capable of being protonated.

Examples of cationic lipids include, but are not limited to 1,2-dioleoyl-3-trimethylammonium propane (DOTAP); N,N-dimethyl-2,3-dioleyloxypropylamine (DODMA), 1,2-di-O-octadecenyl-3-trimethylammonium propane (DOTMA), 3-(N-(N′,N′-dimethylaminoethane)-carbamoyl) cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy) propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2 (spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy) propane (CLinDMA), 2-[5′-(cholest-5-en-3-beta-oxy)-3′-oxapentoxy)-3-dimethyl-1-(cis,cis-9′,12′-octadecadienoxy) propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (βAE-DMRIE), N-(4-carboxybenzyl)-N,N-dimethyl-2,3-bis(oleoyloxy) propan-1-aminium (DOBAQ), 2- ({8-[(3B)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di [oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy) propan-1-aminium bromide (DMORIE), di((Z)-non-2-en-1-yl) 8,8′-((((2 (dimethylamino)ethyl)thio) carbonyl)azanediyl)dioctanoate (ATX), N,N-dimethyl-2,3-bis(dodecyloxy) propan-1-amine (DLDMA), N,N-dimethyl-2,3-bis(tetradecyloxy) propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2- {(2-dodecylcarbamoyl-ethyl)-[2-(2-dodecylcarbamoyl-ethylamino)-ethyl]-amino}-ethylamino) propionamide (lipidoid 98N12-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200), LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy) propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.) or any combination of any of the foregoing. Further suitable cationic lipids for use in the present disclosure include those described in WO2020/128031 and US20200163878, the entire contents of each of which are incorporated herein by reference for the purposes described herein. Further suitable cationic lipids for use in the present disclosure include those described in WO2010/053572 (including Cl 2-200 described at paragraph [00225]) and WO2012/170930, both of which are incorporated herein by reference for the purposes described herein. Additional suitable cationic lipids for use in the present disclosure include HGT4003, HGT5000, HGTS001, HGT5001, HGT5002 (see US20150140070A1, which is incorporated herein by reference in its entirety).

In some embodiments, formulations that are useful for pharmaceutical compositions (e.g., immunogenic compositions, e.g., vaccines) compositions as described herein can comprise at least one cationic lipid. Representative cationic lipids include, but are not limited to, 1,2-dilinoleyoxy-3-(dimethylamino) acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.CI), 1,2-dilinoleoyl-3-trimethylaminopropane chloride salt (DLin-TAP.CI), 1, 2-dilinoleyloxy-3-(N-methylpiperazino) propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino) ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA); dilinoleyl-methyl-4-dimethylaminobutyrate (DLin-MC3-DMA); MC3 (US20100324120, which is incorporated herein by reference in its entirety).

In some embodiments, amino or cationic lipids useful in accordance with the present disclosure have at least one protonatable or deprotonatable group, such that the lipid is positively charged at a pH at or below physiological pH (e.g., pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of lipids have to be present in the charged or neutral form. Lipids having more than one protonatable or deprotonatable group, or which are zwitterionic, are not excluded and may likewise be suitable in the context of the present invention.

In some embodiments, a protonatable lipid has a pKa of the protonatable group in the range of about 4 to about 11, e.g., a pKa of about 5 to about 7.

In some embodiments, a cationic lipid may comprise from about 10 mol % to about 100 mol %, about 20 mol % to about 100 mol %, about 30 mol % to about 100 mol %, about 40 mol % to about 100 mol %, or about 50 mol % to about 100 mol % of total lipid present in a lipid composition utilized in accordance with the present disclosure.

### 3. Additional Lipids or Lipid-Like Materials

In some embodiments, formulations utilized in accordance with the present disclosure may comprise lipids or lipid-like materials other than cationic or cationically ionizable lipids or lipid-like materials, i.e., non-cationic lipids or lipid-like materials (including non-cationically ionizable lipids or lipid-like materials). Collectively, anionic and neutral lipids or lipid-like materials are referred to herein as non-cationic lipids or lipid-like materials. In some embodiments, optimizing a formulation of nucleic acid particles by addition of other hydrophobic moieties, such as cholesterol and lipids, in addition to an ionizable/cationic lipid or lipid-like material may, for example, enhance particle stability and efficacy of nucleic acid delivery.

In some embodiments, a lipid or lipid-like material may be incorporated which may or may not affect the overall charge of particles. In certain embodiments, such lipid or lipid-like material is a non-cationic lipid or lipid-like material.

In some embodiments, a non-cationic lipid may comprise, e.g., one or more anionic lipids and/or neutral lipids. An “anionic lipid” is negatively charged (e.g., at a selected pH).

A “neutral lipid” exists either in an uncharged or neutral zwitterionic form (e.g., at a selected pH). In some embodiments, a formulation comprises one of the following neutral lipid components: (1) a phospholipid, (2) cholesterol or a derivative thereof; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Specific example phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), and further phosphatidylethanolamine lipids with different hydrophobic chains.

In certain embodiments, a formulation utilized in accordance with the present disclosure includes DSPC or DSPC and cholesterol.

In certain embodiments, formulations utilized in accordance with the present disclosure include both a cationic lipid and an additional (non-cationic) lipid.

In some embodiments, formulations herein include a polymer conjugated lipid such as a pegylated lipid. “Pegylated lipids” comprise both a lipid portion and a polyethylene glycol portion. Pegylated lipids are known in the art.

Without wishing to be bound by theory, the amount of (total) cationic lipid compared to the amount of other lipid(s) in formulation may affect important characteristics, such as charge, particle size, stability, tissue selectivity, and bioactivity of the nucleic acid. In some embodiments, the molar ratio of the at least one cationic lipid to the at least one additional lipid is from about 10:0 to about 1:9, about 4:1 to about 1:2, or about 3:1 to about 1:1.

In some embodiments, a non-cationic lipid, in particular a neutral lipid, (e.g., one or more phospholipids and/or cholesterol) may comprise from about 0 mol % to about 90 mol %, from about 0 mol % to about 80 mol %, from about 0 mol % to about 70 mol %, from about 0 mol % to about 60 mol %, or from about 0 mol % to about 50 mol %, of the total lipid present in a formulation.

### 4. Lipoplex Particles

In certain embodiments of the present disclosure, the RNA described herein may be present in RNA lipoplex particles.

An “RNA lipoplex particle” contains lipid, in particular cationic lipid, and RNA. Electrostatic interactions between positively charged liposomes and negatively charged RNA results in complexation and spontaneous formation of RNA lipoplex particles. Positively charged liposomes may be generally synthesized using a cationic lipid, such as DOTMA, and additional lipids, such as DOPE. In one embodiment, an RNA lipoplex particle is a nanoparticle.

In certain embodiments, RNA lipoplex particles include both a cationic lipid and an additional lipid. In some embodiments, a cationic lipid is DOTMA and the additional lipid is DOPE.

In some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In some embodiments, the molar ratio of the at least one cationic lipid to the at least one additional lipid is about 2:1.

In some embodiments, RNA lipoplex particles have an average diameter that in one embodiment ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In specific embodiments, the RNA lipoplex particles have an average diameter of about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm. In an embodiment, the RNA lipoplex particles have an average diameter that ranges from about 250 nm to about 700 nm. In another embodiment, the RNA lipoplex particles have an average diameter that ranges from about 300 nm to about 500 nm. In some embodiments, RNA lipoplex particles have an average diameter of about 400 nm.

RNA lipoplex particles and compositions comprising RNA lipoplex particles described herein are useful for delivery of RNA to a target tissue after parenteral administration, in particular after intravenous administration. The RNA lipoplex particles may be prepared using liposomes that may be obtained by injecting a solution of the lipids in ethanol into water or a suitable aqueous phase. In one embodiment, the aqueous phase has an acidic pH. In one embodiment, the aqueous phase comprises acetic acid, e.g., in an amount of about 5 mM. Liposomes may be used for preparing RNA lipoplex particles by mixing the liposomes with RNA. In one embodiment, the liposomes and RNA lipoplex particles comprise at least one cationic lipid and at least one additional lipid. In one embodiment, the at least one cationic lipid comprises 1,2-di-O-octadecenyl-3-trimethylammonium propane (DOTMA) and/or 1,2-dioleoyl-3-trimethylammonium-propane (DOTAP). In one embodiment, the at least one additional lipid comprises 1,2-di-(9Z-octadecenoyl)-sn-glycero-3-phosphoethanolamine (DOPE), cholesterol (Chol) and/or 1,2-dioleoyl-sn-glycero-3-phosphocholine (DOPC). In one embodiment, the at least one cationic lipid comprises 1,2-di-O-octadecenyl-3-trimethylammonium propane (DOTMA) and the at least one additional lipid comprises 1,2-di-(9Z-octadecenoyl)-sn-glycero-3-phosphoethanolamine (DOPE). In one embodiment, the liposomes and RNA lipoplex particles comprise 1,2-di-O-octadecenyl-3-trimethylammonium propane (DOTMA) and 1,2-di-(9Z-octadecenoyl)-sn-glycero-3-phosphoethanolamine (DOPE).

Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of the RNA lipoplex particles, RNA accumulation and/or RNA expression in the spleen occurs. Thus, RNA lipoplex particles of the disclosure may be used for expressing RNA in the spleen. In an embodiment, after administration of the RNA lipoplex particles, no or essentially no RNA accumulation and/or RNA expression in the lung and/or liver occurs. In one embodiment, after administration of the RNA lipoplex particles, RNA accumulation and/or RNA expression in antigen presenting cells, such as professional antigen presenting cells in the spleen occurs. Thus, RNA lipoplex particles of the disclosure may be used for expressing RNA in such antigen presenting cells. In one embodiment, the antigen presenting cells are dendritic cells and/or macrophages.

### 5. Lipid Nanoparticles (LNPs)

In some embodiments, nucleic acid such as RNA described herein is administered in the form of lipid nanoparticles (LNPs). In some embodiments, LNPs may comprise any lipid capable of forming a particle to which the one or more nucleic acid molecules are attached, or in which the one or more nucleic acid molecules are encapsulated.

In some embodiments, an LNP comprises one or more cationic lipids, and one or more stabilizing lipids. Stabilizing lipids include neutral lipids and pegylated lipids.

In some embodiments, an LNP comprises a cationic lipid, a neutral lipid, a sterol, a polymer conjugated lipid; and an RNA, encapsulated within or associated with the lipid nanoparticle.

In some embodiments, a neutral lipid is selected from the group consisting of DSPC, DPPC, DMPC, DOPC, POPC, DOPE, DOPG, DPPG, POPE, DPPE, DMPE, DSPE, and SM. In some embodiments, the neutral lipid is selected from the group consisting of DSPC, DPPC, DMPC, DOPC, POPC, DOPE and SM. In some embodiments, the neutral lipid is DSPC.

In some embodiments, a sterol is cholesterol.

In some embodiments, a polymer conjugated lipid is a pegylated lipid. In some embodiments, a pegylated lipid has the following structure:

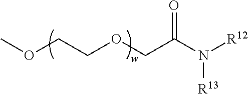

or a pharmaceutically acceptable salt, tautomer or stereoisomer thereof, wherein: R¹² and R¹³ are each independently a straight or branched, saturated or unsaturated alkyl chain containing from 10 to 30 carbon atoms, wherein the alkyl chain is optionally interrupted by one or more ester bonds; and w has a mean value ranging from 30 to 60. In some embodiments, R¹² and R¹³ are each independently straight, saturated alkyl chains containing from 12 to 16 carbon atoms. In some embodiments, w has a mean value ranging from 40 to 55. In some embodiments, the average w is about 45. In some embodiments, R¹² and R¹³ are each independently a straight, saturated alkyl chain containing about 14 carbon atoms, and w has a mean value of about 45.

In some embodiments, a pegylated lipid is DMG-PEG 2000, e.g., having the following structure:

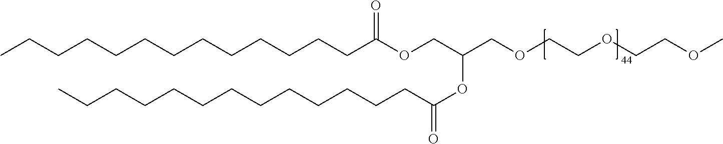

In some embodiments, a cationic lipid component of LNPs has the structure of Formula (III):

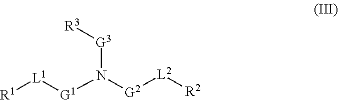

or a pharmaceutically acceptable salt, tautomer, prodrug or stereoisomer thereof, wherein:
one of L1 or L2 is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In some of the foregoing embodiments of Formula (III), the lipid has one of the following structures (IIIA) or (IIIB):

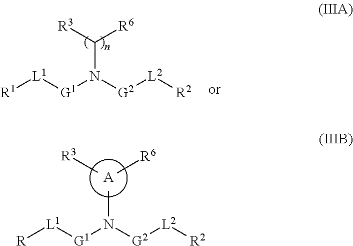

wherein:
A is a 3 to 8-membered cycloalkyl or cycloalkylene ring;
R⁶ is, at each occurrence, independently H, OH or C₁-C₂₄ alkyl; and
n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

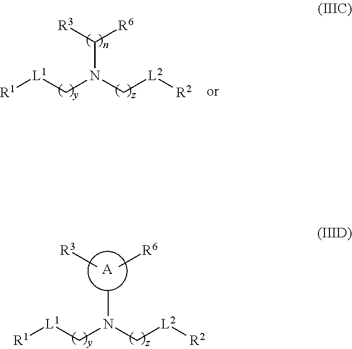

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)O—.

In some different embodiments of Formula (III), the lipid has one of the following structures (IIIE) or (IIIF):

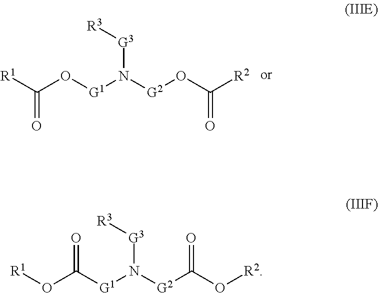

In some of the foregoing embodiments of Formula (III), the lipid has one of the following structures (IIIG), (IIIH), (IIII), or (IIIJ):

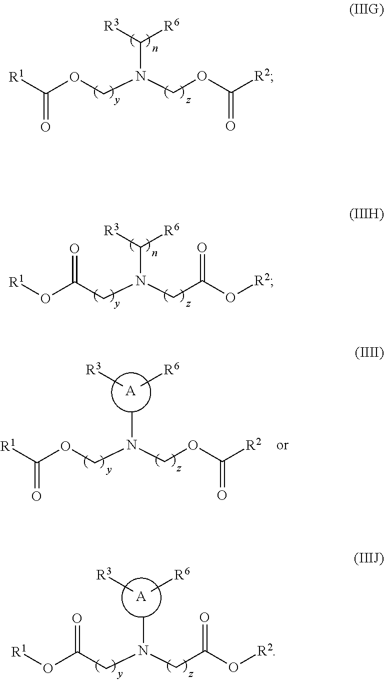

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R⁶ is H. In other of the foregoing embodiments, R⁶ is C₁-C₂₄ alkyl. In other embodiments, R⁶ is OH.

In some embodiments of Formula (III), G³ is unsubstituted. In other embodiments, G3 is substituted. In various different embodiments, G³ is linear C₁-C₂₄ alkylene or linear C₁-C₂₄ alkenylene.

In some other foregoing embodiments of Formula (III), R¹ or R², or both, is C₆-C₂₄ alkenyl. For example, in some embodiments, R¹ and R² each, independently have the following structure:

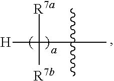

wherein:
R^{7a} and R^{7b} are, at each occurrence, independently H or C₁-C₁₂ alkyl; and
a is an integer from 2 to 12, and
wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

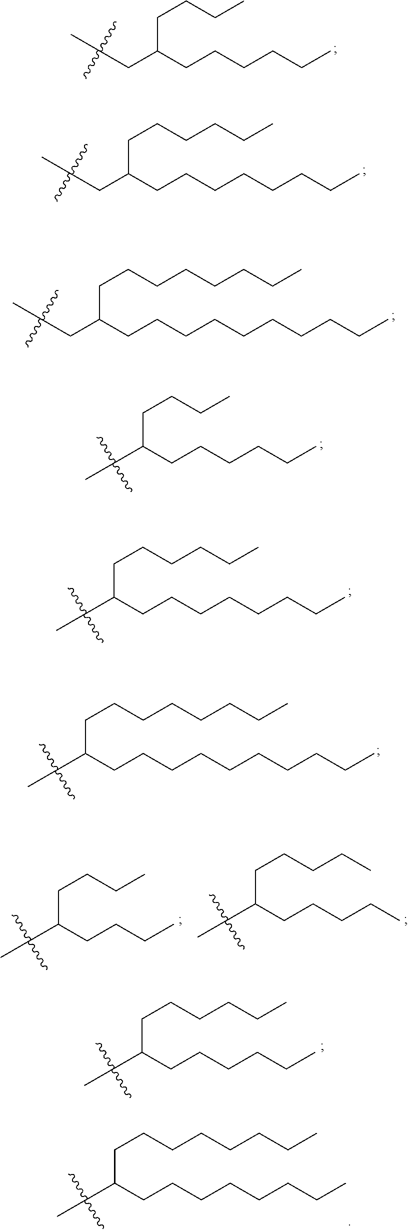

In some of the foregoing embodiments of Formula (III), R³ is OH, CN, —C(═O)OR⁴, —OC(═O)R⁴ or —NHC(═O)R⁴. In some embodiments, R⁴ is methyl or ethyl.

In various different embodiments, the cationic lipid of Formula (III) has one of the structures set forth in in Table 15 below.

TABLE 15
Example Compounds of Formula (III).

| No. | Structure |
|---|---|
| III-1 | 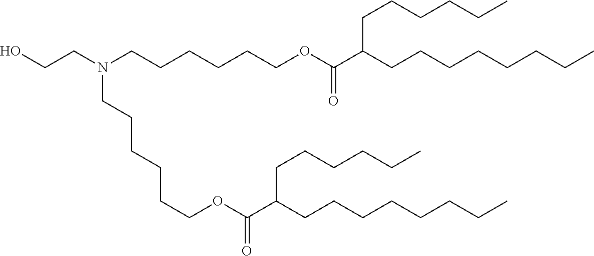 [SMILES: CCCCCCCCC(CCCCCC)C(=O)OCCCCCCN(CCO)CCCCCCOC(=O)C(CCCCCC)CCCCCCCC] |
| III-2 | 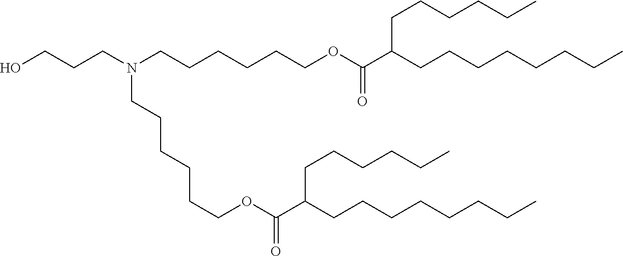 [SMILES: CCCCCCCCC(CCCCCC)C(=O)OCCCCCCN(CCCO)CCCCCCOC(=O)C(CCCCCC)CCCCCCCC] |
| III-3 | 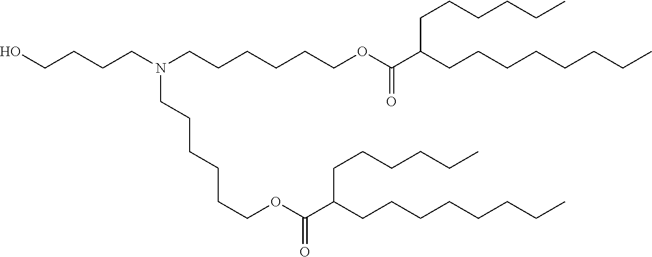 [SMILES: CCCCCCCCC(CCCCCC)C(=O)OCCCCCCN(CCCCO)CCCCCCOC(=O)C(CCCCCC)CCCCCCCC] |
| III-4 | 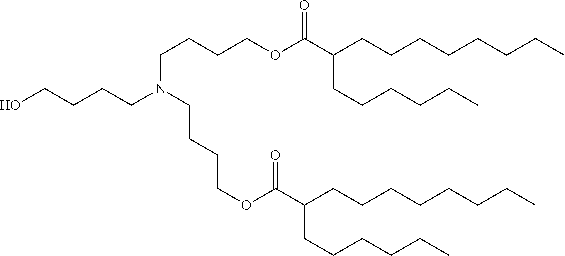 [SMILES: CCCCCCCCC(CCCCCC)C(=O)OCCCCN(CCCCO)CCCCOC(=O)C(CCCCCC)CCCCCCCC] |
| III-5 | 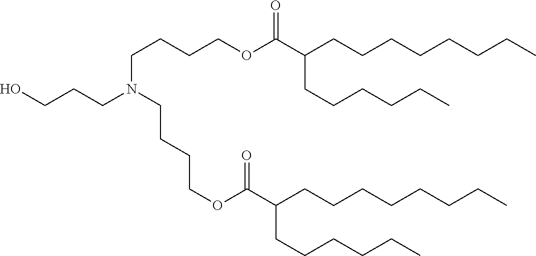 [SMILES: CCCCCCCCC(CCCCCC)C(=O)OCCCCN(CCCO)CCCCOC(=O)C(CCCCCC)CCCCCCCC] |
| III-6 | 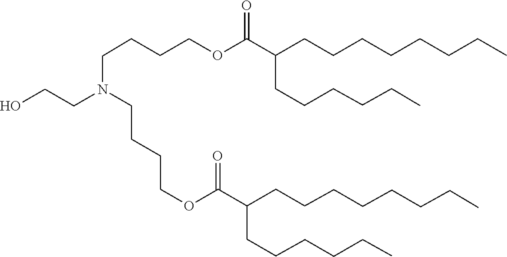 [SMILES: CCCCCCCCC(CCCCCC)C(=O)OCCCCN(CCO)CCCCOC(=O)C(CCCCCC)CCCCCCCC] |
| III-7 | 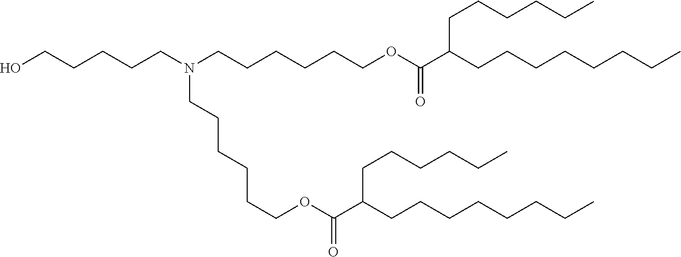 [SMILES: CCCCCCCCC(CCCCCC)C(=O)OCCCCCCN(CCCCCO)CCCCCCOC(=O)C(CCCCCC)CCCCCCCC] |
| III-8 | 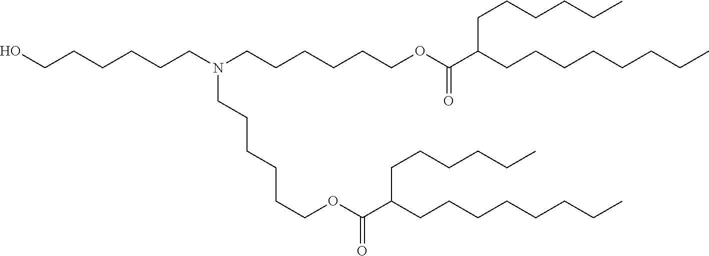 [SMILES: CCCCCCCCC(CCCCCC)C(=O)OCCCCCCN(CCCCCCO)CCCCCCOC(=O)C(CCCCCC)CCCCCCCC] |
| III-9 | 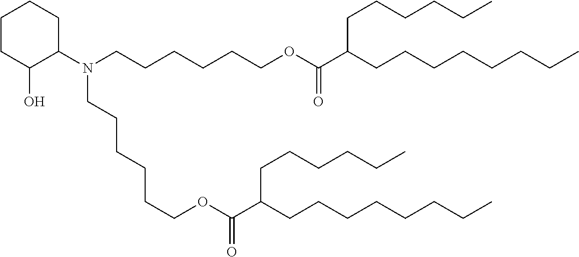 [SMILES: CCCCCCCCC(CCCCCC)C(=O)OCCCCCCN(CCCCCCOC(=O)C(CCCCCC)CCCCCCCC)C1CCCCC1O] |
| III-10 | 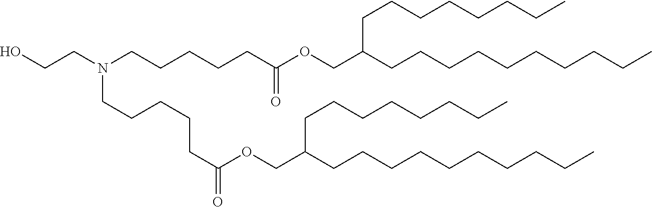 [SMILES: CCCCCCCCCCC(CCCCCCCC)COC(=O)CCCCCN(CCO)CCCCCC(=O)OCC(CCCCCCCC)CCCCCCCCCC] |
| III-11 | 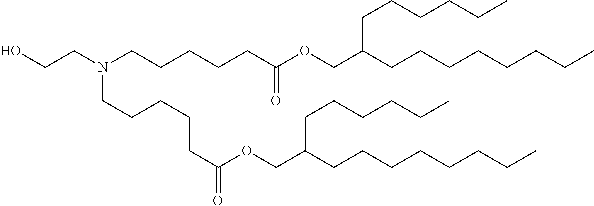 [SMILES: CCCCCCCCC(CCCCCC)COC(=O)CCCCCN(CCO)CCCCCC(=O)OCC(CCCCCC)CCCCCCCC] |
| III-12 | 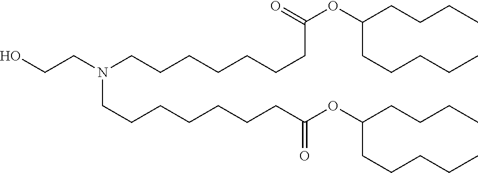 [SMILES: CCCCCC(CCCC)OC(=O)CCCCCCCN(CCO)CCCCCCCC(=O)OC(CCCC)CCCCC] |
| III-13 | 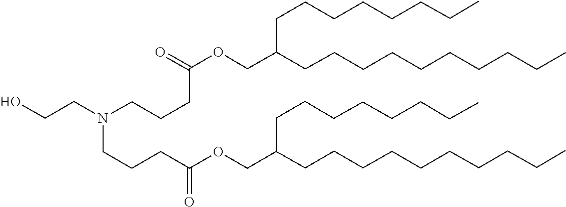 [SMILES: CCCCCCCCCCC(CCCCCCCC)COC(=O)CCCN(CCO)CCCC(=O)OCC(CCCCCCCC)CCCCCCCCCC] |
| III-14 | 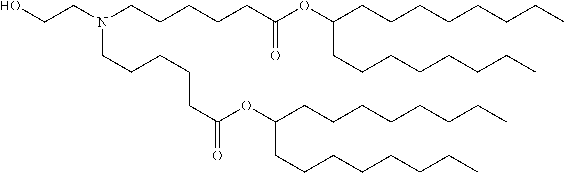 [SMILES: CCCCCCCCC(CCCCCCCC)OC(=O)CCCCCN(CCO)CCCCCC(=O)OC(CCCCCCCC)CCCCCCCC] |
| III-15 | 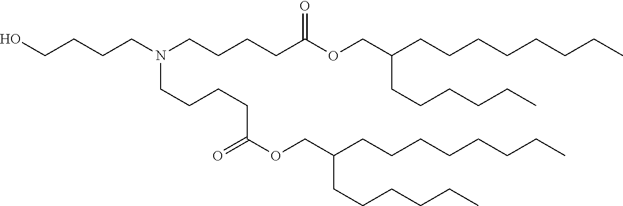 [SMILES: CCCCCCCCC(CCCCCC)COC(=O)CCCCN(CCCCO)CCCCC(=O)OCC(CCCCCC)CCCCCCCC] |
| III-16 | 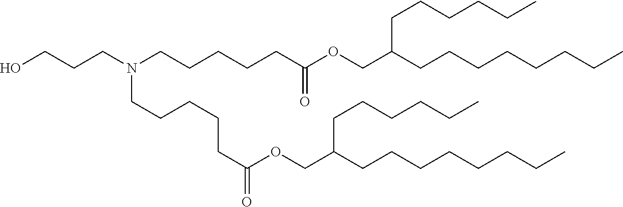 [SMILES: CCCCCCCCC(CCCCCC)COC(=O)CCCCCN(CCCO)CCCCCC(=O)OCC(CCCCCC)CCCCCCCC] |
| III-17 | 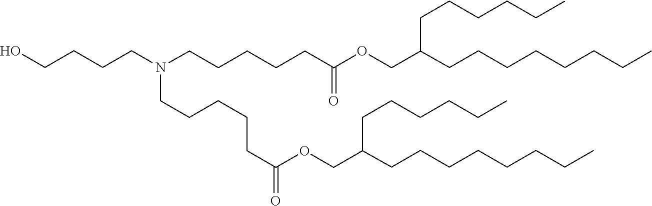 [SMILES: CCCCCCCCC(CCCCCC)COC(=O)CCCCCN(CCCCO)CCCCCC(=O)OCC(CCCCCC)CCCCCCCC] |
| III-18 | 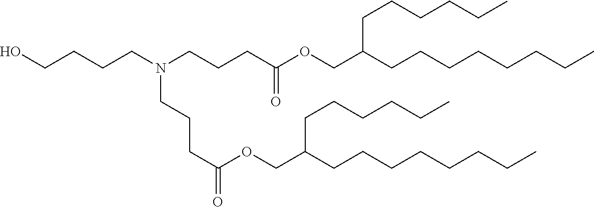 [SMILES: CCCCCCCCC(CCCCCC)COC(=O)CCCN(CCCCO)CCCC(=O)OCC(CCCCCC)CCCCCCCC] |
| III-19 | 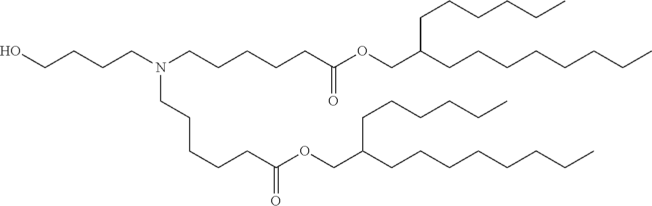 [SMILES: CCCCCCCCC(CCCCCC)COC(=O)CCCCCN(CCCCO)CCCCCC(=O)OCC(CCCCCC)CCCCCCCC] |
| III-20 | 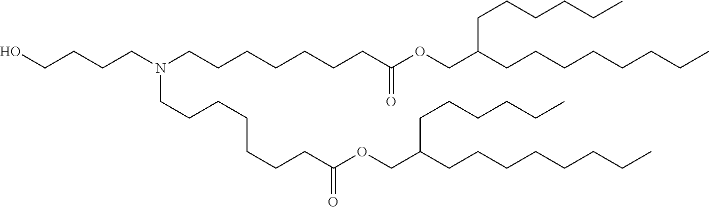 [SMILES: CCCCCCCCC(CCCCCC)COC(=O)CCCCCCCN(CCCCO)CCCCCCCC(=O)OCC(CCCCCC)CCCCCCCC] |
| III-21 | 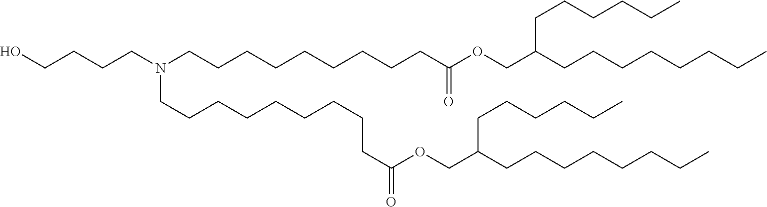 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 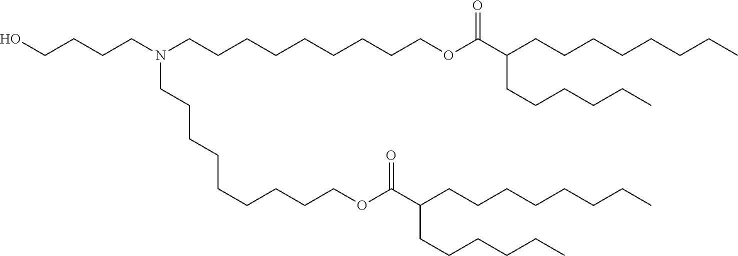 [SMILES: CCCCCCCCC(CCCCCC)C(=O)OCCCCCCCCCN(CCCCO)CCCCCCCCCOC(=O)C(CCCCCC)CCCCCCCC] |
| III-23 | 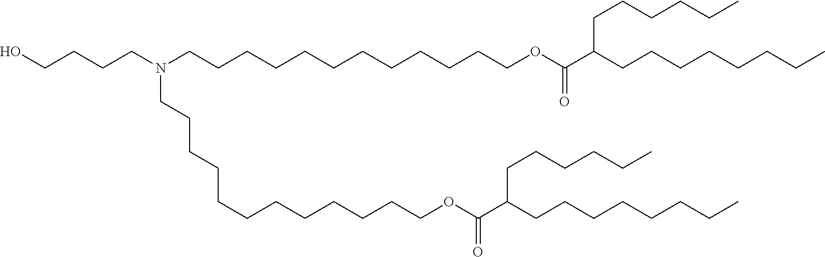 [SMILES: CCCCCCCCC(CCCCCC)C(=O)OCCCCCCCCCCCCN(CCCCO)CCCCCCCCCCCCOC(=O)C(CCCCCC)CCCCCCCC] |
| III-24 | 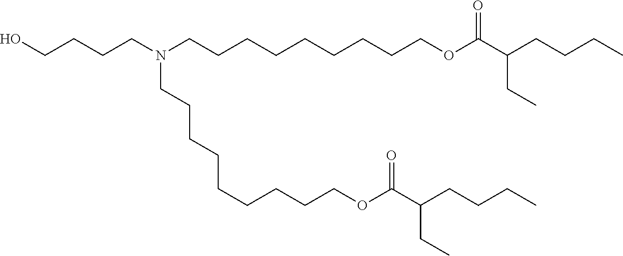 [SMILES: CCCCC(CC)C(=O)OCCCCCCCCCN(CCCCO)CCCCCCCCCOC(=O)C(CC)CCCC] |
| III-25 | 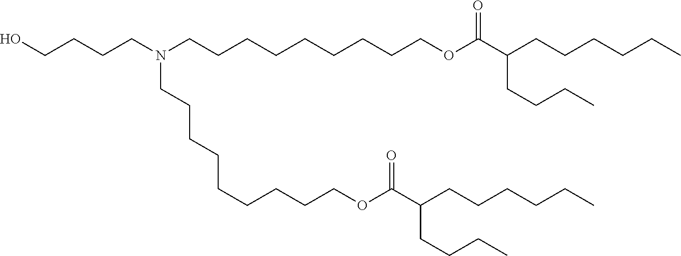 [SMILES: CCCCCCC(CCCC)C(=O)OCCCCCCCCCN(CCCCO)CCCCCCCCCOC(=O)C(CCCC)CCCCCC] |
| III-26 | 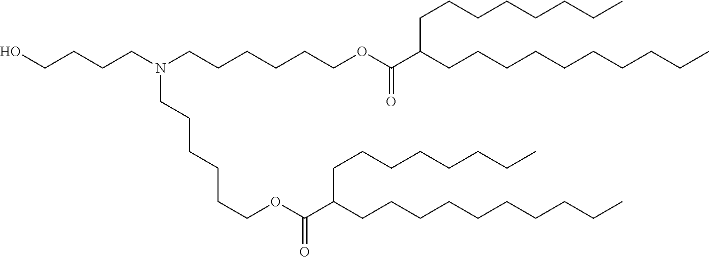 [SMILES: CCCCCCCCCCC(CCCCCCCC)C(=O)OCCCCCCN(CCCCO)CCCCCCOC(=O)C(CCCCCCCC)CCCCCCCCCC] |
| III-27 | 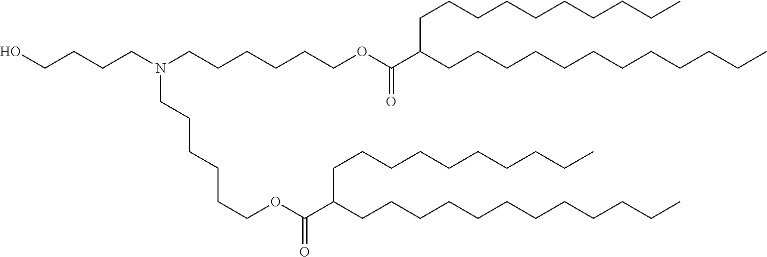 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| III-28 | 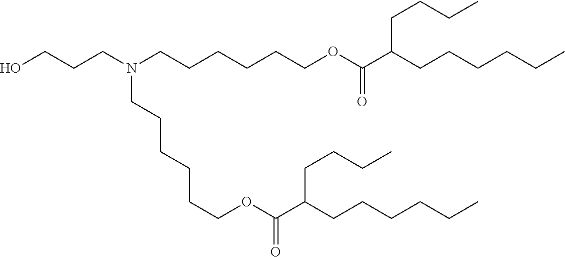 [SMILES: CCCCCCC(CCCC)C(=O)OCCCCCCN(CCCO)CCCCCCOC(=O)C(CCCC)CCCCCC] |
| III-29 | 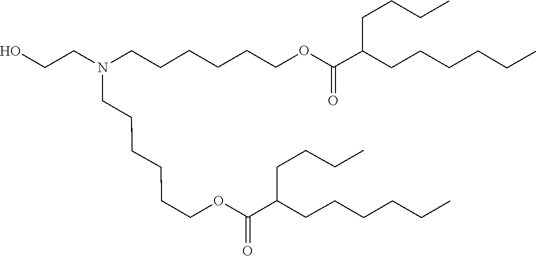 [SMILES: CCCCCCC(CCCC)C(=O)OCCCCCCN(CCO)CCCCCCOC(=O)C(CCCC)CCCCCC] |
| III-30 | 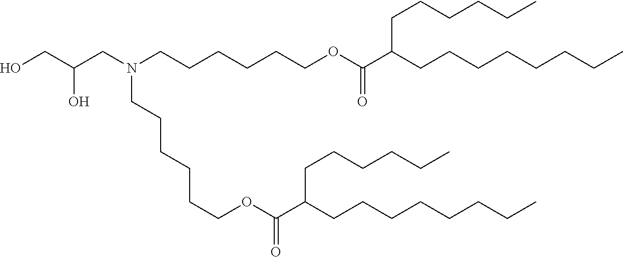 [SMILES: CCCCCCCCC(CCCCCC)C(=O)OCCCCCCN(CCCCCCOC(=O)C(CCCCCC)CCCCCCCC)CC(O)CO] |
| III-31 | 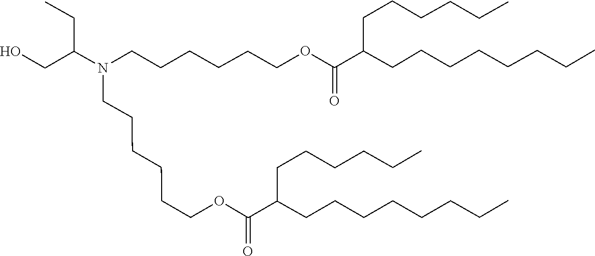 [SMILES: CCCCCCCCC(CCCCCC)C(=O)OCCCCCCN(CCCCCCOC(=O)C(CCCCCC)CCCCCCCC)C(CC)CO] |
| III-32 | 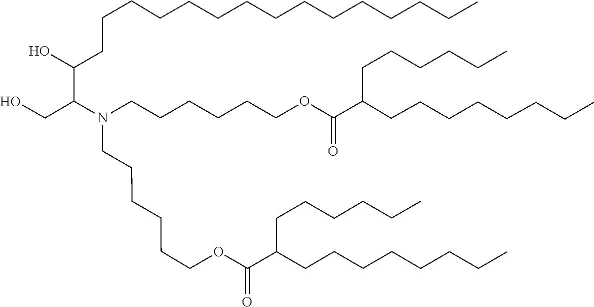 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 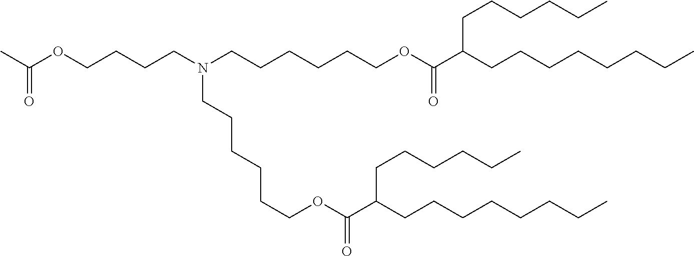 [SMILES: CCCCCCCCC(CCCCCC)C(=O)OCCCCCCN(CCCCCCOC(=O)C(CCCCCC)CCCCCCCC)CCCCOC(C)=O] |
| III-34 | 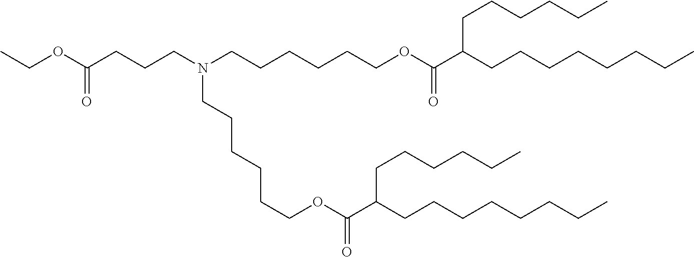 [SMILES: CCCCCCCCC(CCCCCC)C(=O)OCCCCCCN(CCCCCCOC(=O)C(CCCCCC)CCCCCCCC)CCCC(=O)OCC] |
| III-35 | 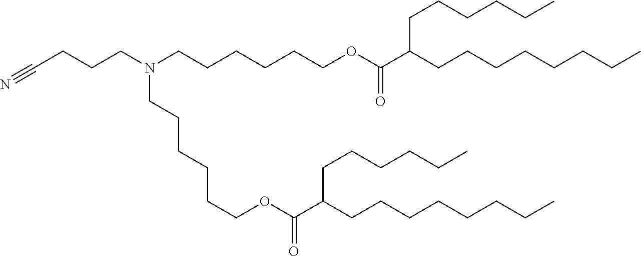 [SMILES: CCCCCCCCC(CCCCCC)C(=O)OCCCCCCN(CCCC#N)CCCCCCOC(=O)C(CCCCCC)CCCCCCCC] |
| III-36 | 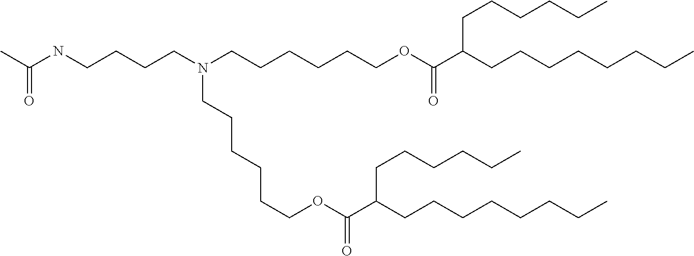 [SMILES: CCCCCCCCC(CCCCCC)C(=O)OCCCCCCN(CCCCCCOC(=O)C(CCCCCC)CCCCCCCC)CCCCNC(C)=O] |

In various different embodiments, a cationic lipid has one of the structures set forth in Table 16 below.

TABLE 16
Example Cationic Lipid Structures

| No. | Structure |
|---|---|
| A | 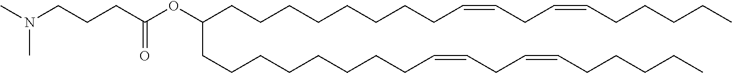 [SMILES: CCCCC/C=C\C/C=C\CCCCCCCCC(CCCCCCCC/C=C\C/C=C\CCCCC)OC(=O)CCCN(C)C] |
| B | 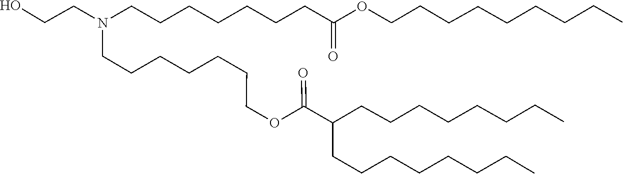 [SMILES: CCCCCCCCCOC(=O)CCCCCCCN(CCO)CCCCCCCOC(=O)C(CCCCCCCC)CCCCCCCC] |
| C | 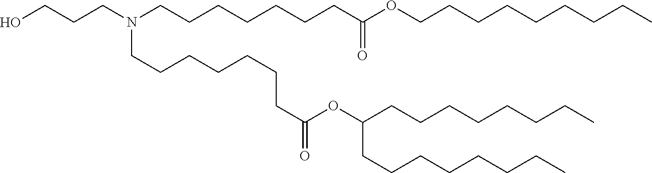 [SMILES: CCCCCCCCCOC(=O)CCCCCCCN(CCCO)CCCCCCCC(=O)OC(CCCCCCCC)CCCCCCCC] |
| D | 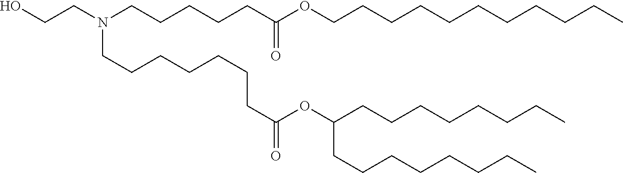 [SMILES: CCCCCCCCCCCOC(=O)CCCCCN(CCO)CCCCCCCC(=O)OC(CCCCCCCC)CCCCCCCC] |
| E | 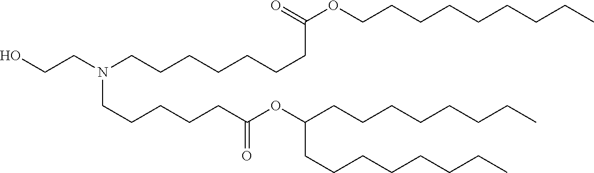 [SMILES: CCCCCCCCCOC(=O)CCCCCCCN(CCO)CCCCCC(=O)OC(CCCCCCCC)CCCCCCCC] |
| F | 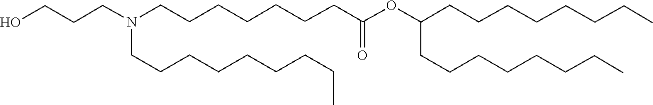 [SMILES: CCCCCCCCCN(CCCO)CCCCCCCC(=O)OC(CCCCCCCC)CCCCCCCC] |

In some embodiments, an LNP comprises a cationic lipid that is an ionizable lipid-like material (lipidoid). In some embodiments, a cationic lipid has the following structure:

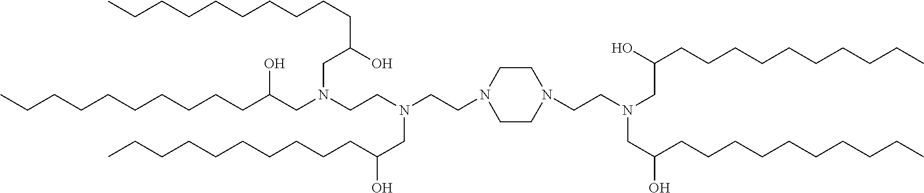 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments, lipid nanoparticles can have an average size (e.g., mean diameter) of about 30 nm to about 150 nm, about 40 nm to about 150 nm, about 50 nm to about 150 nm, about 60 nm to about 130 nm, about 70 nm to about 110 nm, about 70 nm to about 100 nm, about 70 to about 90 nm, or about 70 nm to about 80 nm. In some embodiments, lipid nanoparticles in accordance with the present disclosure can have an average size (e.g., mean diameter) of about 50 nm to about 100 nm. In some embodiments, lipid nanoparticles may have an average size (e.g., mean diameter) of about 50 nm to about 150 nm. In some embodiments, lipid nanoparticles may have an average size (e.g., mean diameter) of about 60 nm to about 120 nm. In some embodiments, lipid nanoparticles in accordance with the present disclosure can have an average size (e.g., mean diameter) of about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. The term “average diameter” or “mean diameter” refers to the mean hydrodynamic diameter of particles as measured by dynamic laser light scattering (DLS) with data analysis using the so-called cumulant algorithm, which provides as results the so-called Z-average with the dimension of a length, and the polydispersity index (PI), which is dimensionless (Koppel, D., J. Chem. Phys. 57, 1972, pp 4814-4820, ISO 13321, which is herein incorporated by reference). Here “average diameter,” “mean diameter,” “diameter,” or “size” for particles is used synonymously with this value of the Z-average.

In some embodiments, lipid nanoparticles described herein may exhibit a polydispersity index less than about 0.5, less than about 0.4, less than about 0.3, or about 0.2 or less. By way of example, lipid nanoparticles can exhibit a polydispersity index in a range of about 0.1 to about 0.3 or about 0.2 to about 0.3. The “polydispersity index” is preferably calculated based on dynamic light scattering measurements by the so-called cumulant analysis as mentioned in the definition of the “average diameter.” Under certain prerequisites, it can be taken as a measure of the size distribution of an ensemble of ribonucleic acid nanoparticles (e.g., ribonucleic acid nanoparticles).

Lipid nanoparticles described herein can be characterized by an “N/P ratio,” which is the molar ratio of cationic (nitrogen) groups (the “N” in N/P) in the cationic polymer to the anionic (phosphate) groups (the “P” in N/P) in RNA. It is understood that a cationic group is one that is either in cationic form (e.g., N⁺), or one that is ionizable to become cationic. Use of a single number in an N/P ratio (e.g., an N/P ratio of about 5) is intended to refer to that number over 1, e.g., an N/P ratio of about 5 is intended to mean 5:1. In some embodiments, a lipid nanoparticle described herein has an N/P ratio greater than or equal to 5. In some embodiments, a lipid nanoparticle described herein has an N/P ratio that is about 5, 6, 7, 8, 9, or 10. In some embodiments, an N/P ratio for a lipid nanoparticle described herein is from about 10 to about 50. In some embodiments, an N/P ratio for a lipid nanoparticle described herein is from about 10 to about 70. In some embodiments, an N/P ratio for a lipid nanoparticle described herein is from about 10 to about 120.

### B. Example Methods of Making Lipid Nanoparticles

Lipids and lipid nanoparticles comprising nucleic acids and their method of preparation are known in the art, including, e.g., as described in U.S. Pat. Nos. 8,569,256, 5,965,542 and U.S. Patent Publication Nos. 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583, 2011/0311582, 2011/0262527, 2011/0216622, 2011/0117125, 2011/0091525, 2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos. WO 99/39741, WO 2018/081480, WO 2017/004143, WO 2017/075531, WO 2015/199952, WO 2014/008334, WO 2013/086373, WO 2013/086322, WO 2013/016058, WO 2013/086373, WO2011/141705, and WO 2001/07548, the full disclosures each of which are herein incorporated by reference in their entirety for the purposes described herein.

For example, in some embodiments, cationic lipids, neutral lipids (e.g., DSPC, and/or cholesterol) and polymer-conjugated lipids can be solubilized in ethanol at a pre-determined molar ratio (e.g., ones described herein). In some embodiments, lipid nanoparticles (lipid nanoparticle) are prepared at a total lipid to polyribonucleotides weight ratio of approximately 10:1 to 30:1. In some embodiments, such polyribonucleotides can be diluted to 0.2 mg/mL in acetate buffer.

In some embodiments, using an ethanol injection technique, a colloidal lipid dispersion comprising polyribonucleotides can be formed as follows: an ethanol solution comprising lipids, such as cationic lipids, neutral lipids, and polymer-conjugated lipids, is injected into an aqueous solution comprising polyribonucleotides (e.g., ones described herein).

In some embodiments, lipid and polyribonucleotide solutions can be mixed at room temperature by pumping each solution at controlled flow rates into a mixing unit, for example, using piston pumps. In some embodiments, the flow rates of a lipid solution and an RNA solution into a mixing unit are maintained at a ratio of 1:3. Upon mixing, nucleic acid-lipid particles are formed as the ethanolic lipid solution is diluted with aqueous polyribonucleotides. The lipid solubility is decreased, while cationic lipids bearing a positive charge interact with the negatively charged RNA.

In some embodiments, a solution comprising RNA-encapsulated lipid nanoparticles can be processed by one or more of concentration adjustment, buffer exchange, formulation, and/or filtration.

In some embodiments, RNA-encapsulated lipid nanoparticles can be processed through filtration.

In some embodiments, particle size and/or internal structure of lipid nanoparticles (with or without RNAs) may be monitored by appropriate techniques such as, e.g., small-angle X-ray scattering (SAXS) and/or transmission electron cryomicroscopy (CryoTEM).

### V. Pharmaceutical Compositions

The present disclosure provides compositions, e.g., pharmaceutical compositions comprising one or more polyribonucleotides described herein. Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, an excipient is approved for use in humans and for veterinary use. In some embodiments, an excipient is approved by the United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

General considerations in the formulation and/or manufacture of pharmaceutical agents may be found, for example, in Remington: The Science and Practice of Pharmacy 21st ed., Lippincott Williams & Wilkins, 2005 (incorporated herein by reference).

In some embodiments, pharmaceutical compositions provided herein may be formulated with one or more pharmaceutically acceptable carriers or diluents as well as any other known adjuvants and excipients in accordance with conventional techniques such as those disclosed in Remington: The Science and Practice of Pharmacy 21st ed., Lippincott Williams & Wilkins, 2005 (incorporated herein by reference).

Pharmaceutical compositions described herein can be administered by appropriate methods known in the art. As will be appreciated by a skilled artisan, the route and/or mode of administration may depend on a number of factors, including, e.g., but not limited to stability and/or pharmacokinetics and/or pharmacodynamics of pharmaceutical compositions described herein.

In some embodiments, pharmaceutical compositions described herein are formulated for parenteral administration, which includes modes of administration other than enteral and topical administration, usually by injection, and includes, without limitation, intravenous, intramuscular, intraarterial, intradermal, subcutaneous, subcuticular, or intraarticular injection and infusion. In preferred embodiments, pharmaceutical compositions described herein are formulated for intravenous, intramuscular, or subcutaneous administration. In particularly preferred embodiments, pharmaceutical compositions described herein are formulated for intramuscular administration.

In some embodiments, pharmaceutical compositions described herein are formulated for intravenous administration. In some embodiments, pharmaceutically acceptable excipients that may be useful for intravenous administration include sterile aqueous solutions or dispersions and sterile powders for preparation of sterile injectable solutions or dispersions.

Therapeutic compositions typically must be sterile and stable under the conditions of manufacture and storage. The composition can be formulated as a solution, microemulsion, lipid nanoparticles, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. Proper fluidity can be maintained, for example, by the use of surfactants. In many cases, it will be preferable to include isotonic agents, for example, sugars, polyalcohols such as mannitol, sorbitol, or sodium chloride in the composition. In some embodiments, prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization and/or microfiltration. In some embodiments, pharmaceutical compositions can be prepared as described herein and/or methods known in the art. In some embodiments, a pharmaceutical composition includes ALC-0315; ALC-0159; DSPC; Cholesterol; Sucrose; NaCl; KCl; Na₂HPO₄; KH₂PO₄; Water for injection. In some embodiments, normal saline (isotonic 0.9% NaCl) is used as diluent.

These compositions may also contain adjuvants such as preservatives, wetting agents, emulsifying agents and dispersing agents. Prevention of the presence of microorganisms may be ensured both by sterilization procedures, and by the inclusion of various antibacterial and antifungal agents, for example, paraben, chlorobutanol, phenol sorbic acid, and the like. It may also be desirable to include isotonic agents, such as sugars, sodium chloride, and the like into pharmaceutical compositions described herein. In addition, prolonged absorption of the injectable pharmaceutical form may be brought about by the inclusion of agents which delay absorption such as aluminum monostearate and gelatin.

Formulations of pharmaceutical compositions described herein may be prepared by any method known or hereafter developed in the art of pharmacology. In general, such preparatory methods include the step of bringing active ingredient(s) into association with a diluent or another excipient and/or one or more other accessory ingredients, and then, if necessary and/or desirable, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of at least one RNA product produced using a system and/or method described herein.

Relative amounts of polyribonucleotides encapsulated in lipid nanoparticles, a pharmaceutically acceptable excipient, and/or any additional ingredients in a pharmaceutical composition can vary, depending upon the subject to be treated, target cells, diseases or disorders, and may also further depend upon the route by which the composition is to be administered.

In some embodiments, pharmaceutical compositions described herein are formulated into pharmaceutically acceptable dosage forms by conventional methods known to those of skill in the art. Actual dosage levels of the active ingredients (e.g., polyribonucleotides encapsulated in lipid nanoparticles) in the pharmaceutical compositions described herein may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A physician having ordinary skill in the art can readily determine and prescribe the effective amount of the pharmaceutical composition required. For example, a physician could start doses of active ingredients (e.g., polyribonucleotides encapsulated in lipid nanoparticles) employed in the pharmaceutical composition at levels lower than that required in order to achieve the desired therapeutic effect and gradually increase the dosage until the desired effect is achieved.

In some embodiments, a pharmaceutical composition is formulated (e.g., but not limited to, for intravenous, intramuscular, or subcutaneous administration) to deliver a dose of about 5 mg RNA/kg.

In some embodiments, a pharmaceutical composition described herein may further comprise one or more additives, for example, in some embodiments that may enhance stability of such a composition under certain conditions. Examples of additives may include but are not limited to salts, buffer substances, preservatives, and carriers. For example, in some embodiments, a pharmaceutical composition may further comprise a cryoprotectant (e.g., sucrose) and/or an aqueous buffered solution, which may in some embodiments include one or more salts, including, e.g., alkali metal salts or alkaline earth metal salts such as, e.g., sodium salts, potassium salts, and/or calcium salts.

In some embodiments, a pharmaceutical composition provided herein is a preservative-free, sterile RNA-lipid nanoparticle dispersion in an aqueous buffer for intravenous or intramuscular administration.

Although the descriptions of pharmaceutical compositions provided herein are principally directed to pharmaceutical compositions that are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation.

### A. Certain Example Pharmaceutical Compositions

Provided herein are combinations that comprise two or more polyribonucleotides as described herein. In some embodiments, a combination comprises three or more polyribonucleotides as described herein. In some embodiments, a combination comprises three polyribonucleotides as described herein.

Also provided herein are combinations that comprise two or more pharmaceutical compositions, wherein each pharmaceutical composition comprises a polyribonucleotide as described herein.

Provided herein are combinations that comprise two or more RNA constructs as described herein. In some embodiments, a combination comprises three or more RNA constructs as described herein. In some embodiments, a combination comprises three RNA constructs as described herein.

Also provided herein are combinations that comprise two or more pharmaceutical compositions, wherein each pharmaceutical composition comprises an RNA constructs as described herein.

In some embodiments, a combination comprises a first polyribonucleotide encodes a first polypeptide comprising an HSV-2 gC antigen or antigenic fragment thereof, and a second polyribonucleotide encodes a second polypeptide comprising an HSV-2 gD antigen or antigenic fragment thereof.

In some embodiments, a combination comprises a first polyribonucleotide encodes a first polypeptide comprising an HSV-2 gC antigen, and a second polyribonucleotide encodes a second polypeptide comprising an HSV-2 gD antigen. In some embodiments, the first polypeptide comprises an amino acid sequence according to SEQ ID NO: 65. In some embodiments, the second polypeptide comprises an amino acid sequence according to SEQ ID NO: 70.

In some embodiments, a combination comprises (i) a first pharmaceutical composition comprising a first polyribonucleotide, wherein the first polyribonucleotide encodes a first polypeptide comprising an HSV-2 gC antigen or antigenic fragment thereof, and (ii) a second pharmaceutical composition comprising a second polyribonucleotide, wherein the second polyribonucleotide encodes a second polypeptide comprising an HSV-2 gD antigen or antigenic fragment thereof.

In some embodiments, a combination comprises (i) a first pharmaceutical composition comprising a first polyribonucleotide, wherein the first polyribonucleotide encodes a first polypeptide comprising an HSV-2 gC antigen, and (ii) a second pharmaceutical composition comprising a second polyribonucleotide, wherein the second polyribonucleotide encodes a second polypeptide comprising an HSV-2 gD antigen. In some embodiments, the first polypeptide comprises an amino acid sequence according to SEQ ID NO: 65. In some embodiments, the second polypeptide comprises an amino acid sequence according to SEQ ID NO: 70.

In some embodiments, a combination comprises a first polyribonucleotide encodes a first polypeptide comprising an HSV-2 gC antigen or antigenic fragment thereof, and a second polyribonucleotide encodes a second polypeptide comprising an HSV-2 gE antigen or antigenic fragment thereof.

In some embodiments, a combination comprises a first polyribonucleotide encodes a first polypeptide comprising an HSV-2 gC antigen, and a second polyribonucleotide encodes a second polypeptide comprising an HSV-2 gE antigen. In some embodiments, the first polypeptide comprises an amino acid sequence according to SEQ ID NO: 65. In some embodiments, the second polypeptide comprises an amino acid sequence according to SEQ ID NO: 73.

In some embodiments, a combination comprises (i) a first pharmaceutical composition comprising a first polyribonucleotide, wherein the first polyribonucleotide encodes a first polypeptide comprising an HSV-2 gC antigen or antigenic fragment thereof, and (ii) a second pharmaceutical composition comprising a second polyribonucleotide, wherein the second polyribonucleotide encodes a second polypeptide comprising an HSV-2 gE antigen or antigenic fragment thereof.

In some embodiments, a combination comprises (i) a first pharmaceutical composition comprising a first polyribonucleotide, wherein the first polyribonucleotide encodes a first polypeptide comprising an HSV-2 gC antigen, and (ii) a second pharmaceutical composition comprising a second polyribonucleotide, wherein the second polyribonucleotide encodes a second polypeptide comprising an HSV-2 gE antigen. In some embodiments, the first polypeptide comprises an amino acid sequence according to SEQ ID NO: 65. In some embodiments, the second polypeptide comprises an amino acid sequence according to SEQ ID NO: 73.

In some embodiments, a combination comprises a first polyribonucleotide encodes a first polypeptide comprising an HSV-2 gD antigen or antigenic fragment thereof, and a second polyribonucleotide encodes a second polypeptide comprising an HSV-2 gE antigen or antigenic fragment thereof.

In some embodiments, a combination comprises a first polyribonucleotide encodes a first polypeptide comprising an HSV-2 gD antigen, and a second polyribonucleotide encodes a second polypeptide comprising an HSV-2 gE antigen. In some embodiments, the first polypeptide comprises an amino acid sequence according to SEQ ID NO: 70. In some embodiments, the second polypeptide comprises an amino acid sequence according to SEQ ID NO: 73.

In some embodiments, a combination comprises (i) a first pharmaceutical composition comprising a first polyribonucleotide, wherein the first polyribonucleotide encodes a first polypeptide comprising an HSV-2 gD antigen or antigenic fragment thereof, and (ii) a second pharmaceutical composition comprising a second polyribonucleotide, wherein the second polyribonucleotide encodes a second polypeptide comprising an HSV-2 gE antigen or antigenic fragment thereof.

In some embodiments, a combination comprises (i) a first pharmaceutical composition comprising a first polyribonucleotide, wherein the first polyribonucleotide encodes a first polypeptide comprising an HSV-2 gD antigen, and (ii) a second pharmaceutical composition comprising a second polyribonucleotide, wherein the second polyribonucleotide encodes a second polypeptide comprising an HSV-2 gE antigen. In some embodiments, the first polypeptide comprises an amino acid sequence according to SEQ ID NO: 70. In some embodiments, the second polypeptide comprises an amino acid sequence according to SEQ ID NO: 73.

In some embodiments, a combination comprises a first polyribonucleotide encodes a first polypeptide comprising an HSV-2 gC antigen or antigenic fragment thereof, a second polyribonucleotide encodes a second polypeptide comprising an HSV-2 gD antigen or antigenic fragment thereof, and a third polyribonucleotide encodes a third polypeptide comprising an HSV-2 gE antigen or antigenic fragment thereof.

In some embodiments, a combination comprises a first polyribonucleotide encodes a polypeptide comprising an HSV-2 gC antigen, a second polyribonucleotide encodes a polypeptide comprising an HSV-2 gD antigen, and a third polyribonucleotide encodes a third polypeptide comprising an HSV-2 gE antigen. In some embodiments, the first polypeptide comprises an amino acid sequence according to SEQ ID NO: 65. In some embodiments, the second polypeptide comprises an amino acid sequence according to SEQ ID NO: 70. In some embodiments, the second polypeptide comprises an amino acid sequence according to SEQ ID NO: 73.

In some embodiments, a combination comprises (i) a first pharmaceutical composition comprising a first polyribonucleotide, wherein the first polyribonucleotide encodes a first polypeptide comprising an HSV-2 gC antigen or antigenic fragment thereof, (ii) a second pharmaceutical composition comprising a second polyribonucleotide, wherein the second polyribonucleotide encodes a second polypeptide comprising an HSV-2 gD antigen or antigenic fragment thereof, and (iii) a third pharmaceutical composition comprising a third polyribonucleotide, wherein the third polyribonucleotide encodes a third polypeptide comprising an HSV-2 gE antigen or antigenic fragment thereof.

In some embodiments, a combination comprises (i) a first pharmaceutical composition comprising a first polyribonucleotide, wherein the first polyribonucleotide encodes a first polypeptide comprising an HSV-2 gC antigen, (ii) a second pharmaceutical composition comprising a second polyribonucleotide, wherein the second polyribonucleotide encodes a second polypeptide comprising an HSV-2 gD antigen, and (iii) a third pharmaceutical composition comprising a third polyribonucleotide, wherein the third polyribonucleotide encodes a third polypeptide comprising an HSV-2 gE antigen. In some embodiments, the first polypeptide comprises an amino acid sequence according to SEQ ID NO: 65. In some embodiments, the second polypeptide comprises an amino acid sequence according to SEQ ID NO: 70. In some embodiments, the second polypeptide comprises an amino acid sequence according to SEQ ID NO: 73.

In some embodiments, a polypeptide that comprises an amino acid sequence according to SEQ ID NO: 65 is encoded by a ribonucleic acid sequence according to 106. In some embodiments, a polypeptide that comprises an amino acid sequence according to SEQ ID NO: 70 is encoded by a ribonucleic acid sequence according to 118. In some embodiments, a polypeptide that comprises an amino acid sequence according to SEQ ID NO: 73 is encoded by a ribonucleic acid sequence according to 123.

### VI. Patient Populations

In some aspects, technologies of the present disclosure are used for therapeutic and/or prophylactic purposes. In some embodiments, technologies of the present disclosure are used in the treatment and/or prophylactic of an HSV infection. Prophylactic purposes of the present disclosure comprise pre-exposure prophylaxis and/or post-exposure prophylaxis.

In some embodiments, technologies of the present disclosure are used in the treatment and/or prophylaxis of a disorder related to such an HSV (e.g., HSV-1 and/or HSV-2) infection. A disordered related to such an HSV (e.g., HSV-1 and/or HSV-2) infection comprises, for example, a typical symptom and/or a complication of an HSV (e.g., HSV-1 and/or HSV-2) infection.

In some embodiments, provided compositions (e.g., that are or comprise HSV antigens, HSV gC, gD and/or gE antigens, e.g., HSV-2 gC, gD and/or gE antigens) may be useful to detect and/or characterize one or more features of an anti-HSV (e.g., anti-HSV-1 and/or anti-HSV-2) immune response (e.g., by detecting binding to a provided antigen by serum from an infected subject).

In some embodiments, provided compositions (e.g., that are or comprise HSV antigens, HSV gC, gD and/or gE antigens, e.g., HSV-1 gC, gD and/or gE antigens) are useful to raise antibodies to one or more epitopes included therein; such antibodies may themselves be useful, for example for detection or treatment of an HSV infection.

The present disclosure provides use of encoding nucleic acids (e.g., DNA or RNA) to produce encoded antigens and/or use of DNA constructs to produce RNA.

In some embodiments, technologies of the present disclosure are utilized in a non-limited subject population; in some embodiments, technologies of the present disclosure are utilized in particular subject populations.

In some embodiments, a subject population comprises an adult population. In some embodiments, an adult population comprises subjects between the ages of about 18 years and about 55 years of age (e.g., about 19, 20, 25, 30, 35, 40, 45, 50, 51, 52, 53, 54, or 55 years of age).

In some embodiments, a subject population comprises an elderly population. In some embodiments, an elderly population comprises subjects of, about 56 years of age, about 60 years of age, about 70 years of age, or older (e.g., about 60, 65, 70, 75, 80, 85, 90, 95, or 100 years of age).

In some embodiments, a subject has a weight of at least about 50 kg. In some embodiments, a subject has a weight of at least about 51 kg (e.g., about 52, 53, 54, 55, 56,57, 58, 59, 60 kg).

In some embodiments, a subject has a body mass index (BMI) in a range of about 17.5 kg/m² to about 37 kg/m², such as about 18 kg/m² to about 36 kg/m², such as about 18.5 kg/m² to about 35 kg/m². In some embodiments, a subject has a BMI of at least 17 kg/m², such as at least 17.5 kg/m², such as at least 18 kg/m², such as at least 18.5 kg/m². In some embodiments, a subject has a BMI of at the most 40 kg/m², such as at the most 39 kg/m², such as at the most 38 kg/m², such as at the most 37 kg/m², such as at the most 36 kg/m², such as at the most 35 kg/m².

In some embodiments, a subject population comprises a pediatric population. In some embodiments, a pediatric population comprises subjects approximately 18 years old or younger. In some such embodiments, a pediatric population comprises subjects between the ages of about 1 year and about 18 years (e.g., 1, 2, 3, 4, 5, 6, 7, 8, 9, 10, 11, 12, 13, 14, 15, 16, 17, or 18 years of age).

In some embodiments, a subject population comprises a newborn population. In some embodiments, a newborn population comprises subjects about 12 months or younger (e.g., 12, 11, 10, 9, 8, 7, 6, 5, 4, 3, 2, 1 months or younger). In some embodiments, subject populations to be treated with technologies described herein include infants (e.g., about 12 months or younger) whose mothers did not receive such technologies described herein during pregnancy. In some embodiments, subject populations to be treated with technologies described herein may include pregnant women; in some embodiments, infants whose mothers were treated with disclosed technologies during pregnancy (e.g., who received at least one dose, or alternatively only who received both doses), are not vaccinated during the first weeks, months, or even years (e.g., 1, 2, 3, 4, 5, 6, 7, 8 weeks or more, or 1, 2, 3, 4, 5, 6, 7, 8, 9, 10, 11, 12, 13, 14, 15, 16, 17, 18, 19, 20, 21, 22, 23, 24 months or more, or 1, 2, 3, 4, 5 years or more) post-birth. Alternatively or additionally, in some embodiments, infants whose mothers were treated with disclosed technologies during pregnancy (e.g., who received at least one dose, or alternatively only who received both doses), receive reduced treated with disclosed technologies (e.g., lower doses and/or smaller numbers of administrations—e.g., boosters- and/or lower total exposure over a given period of time) after birth, for example during the first weeks, months, or even years (e.g., 1, 2, 3, 4, 5, 6, 7, 8 weeks or more, or 1, 2, 3, 4, 5, 6, 7, 8, 9, 10, 11, 12, 13, 14, 15, 16, 17, 18, 19, 20, 21, 22, 23, 24 months or more, or 1, 2, 3, 4, 5 years or more) post-birth or may need reduced vaccination (e.g., lower doses and/or smaller numbers of administrations—e.g., boosters-over a given period of time), In some embodiments, compositions as provided herein are administered to subject populations that do not include pregnant women.

In some embodiments, a subject population is or comprises children aged 6 weeks to up to 17 months of age.

In some embodiments, a provided pharmaceutical composition (e.g., immunogenic composition, e.g., vaccine) may be administered in combination with (i.e., so that subject(s) are simultaneously exposed to both) another pharmaceutical composition (e.g., immunogenic composition, e.g., vaccine) or therapeutic intervention, e.g., to treat or prevent an HSV infection, or another disease, disorder, or condition.

In some embodiments, a provided pharmaceutical composition (e.g., immunogenic composition, e.g., vaccine) may be administered with a protein vaccine, a DNA vaccine, an RNA vaccine, a cellular vaccine, a conjugate vaccine, etc. In some embodiments, one or more doses of a provided pharmaceutical composition (e.g., immunogenic composition, e.g., vaccine) may be administered together with (e.g., in a single visit) another vaccine or other therapy.

In some embodiments, a provided pharmaceutical composition (e.g., immunogenic composition, e.g., vaccine) may be administered to subjects who have been exposed, or expect they have been exposed, to HSV (e.g., HSV-1 and/or HSV-2). In some embodiments, a provided pharmaceutical composition (e.g., immunogenic composition, e.g., vaccine) may be administered to subjects who do not have symptoms of an HSV (e.g., HSV-1 and/or HSV-2).

In some embodiments, a subject has no prior history of known or suspected herpes simplex vaccination prior to administration of one or more doses of a composition as disclosed herein.

In some embodiments, a subject does not have febrile illness prior to administration of one or more doses of a composition as disclosed herein. In some embodiments, a subject does not have febrile illness about 72 hours, about 48 hours, about 36 hours, about 24 hours, or about 12 hours prior to administration of one or more doses of a composition as disclosed herein.

In some embodiments, a subject does not have an acute illness prior to administration of one or more doses of a composition as disclosed herein. In some embodiments, a subject does not have an acute illness about 72 hours, about 48 hours, about 36 hours, about 24 hours, or about 12 hours prior to administration of one or more doses of a composition as disclosed herein.

In some embodiments, a subject has not received a vaccine 0 to 300 days, 0 to 290 days, 0 to 280 days, 0 to 270 days, 0 to 260 days, 0 to 250 days, 0 to 240 days, 0 to 230 days, 0 to 220 days, 0 to 210, 0 to 200 days, 0 to 190 days, 0 to 180 days, 0 to 170 days, 0 to 160 days, 0 to 150 days, 0 to 140 days, 0 to 130 days, 0 to 120 days, 0 to 110 days, 0 to 100 days, 0 to 90 days, 0 to 80 days, 0 to 70 days, 0 to 60 days, 0 to 50 days, 0 to 40 days, 0 to 35 days, 0 to 30 days, 0 to 29 days, 0 to 28 days before being administered one or more doses of a therapeutically effective amount of a composition as disclosed herein. In some embodiments, a subject has not received a vaccine about 7 days, about 14 days, about 21 days, about 28 days, about 35 days, about 42 days, about 49 days, about 56 days, about 60 days, about 70 days, about 80 days, about 90 days, about 100 days, about 125 days, about 150 days, about 175 days, about 190 days, about 200 days, about 210 days, or about 210 days before being administered one or more doses of a therapeutically effective amount of a composition as disclosed herein. In some embodiments, a vaccine is not a seasonal influenza vaccine or a medically indicated vaccine.

In some embodiments, a subject does not receive a vaccine at least 2 weeks to 35 weeks, at least 3 weeks to 34 weeks, or at least 4 weeks to 33 weeks after administration of one or more doses of a therapeutically effective amount of a composition as disclosed herein. In some embodiments, a subject does not receive a vaccine at least about 2 weeks, at least about 4 weeks, at least about 6 weeks, at least about 8 weeks, at least about 10 weeks, at least about 12 weeks, at least about 14 weeks, at least about 16 weeks, at least about 18 weeks, at least about 20 weeks, at least about 22 weeks, at least about 24 weeks, at least about 26 weeks, at least about 28 weeks, or at least about 30 weeks after administration of one or more doses of a therapeutically effective amount of a composition as disclosed herein. In some embodiments, the vaccine is not a seasonal influenza vaccine or a medically indicated vaccine.

In some embodiments, a subject has not received blood, plasma products, or immunoglobulins about 0 to 600 days, about 0 to 590 days, about 0 to 580 days, about 0 to 570 days, about 0 to 560 days, about 0 to 550 days, or about 0 to 545 days before administration of one or more doses of a therapeutically effective amount of a composition disclosed herein.

In some embodiments, a subject has not received an allergy treatment 8 to 45 days, 12 to 40 days, 16 to 38 days, 21 to 35 days, 23 to 32 days, 25 to 30 days, or 26 to 29 days before administration of one or more doses of a therapeutically effective amount of a composition disclosed herein. In some embodiments, a subject has not received an allergy treatment about 14 days, about 16 days, about 18 days, about 20 days, about 22 days, about 24 days, about 26 days, about 28 days before administration of one or more doses of a therapeutically effective amount of a composition disclosed herein. In some embodiments, an allergy treatment comprises antigen injections.

In some embodiments, a subject has not received an immunosuppressive medication 7 to 56 days, 14 to 56 days, 21 to 56 days, 28 to 56 days, 35 to 56 days, 42 to 56 days, 49 to 56 days, 7 to 49 days, 14 to 49 days, 21 to 49 days, 28 to 49 days, 35 to 49 days, 42 to 49 days, 7 to 42 days, 14 to 42 days, 21 to 42 days, 28 to 42 days, 35 to 42 days, 7 to 35 days, 14 to 35 days, 21 to 35 days, 28 to 35 days, 7 to 28 days, 14 to 28 days, 21 to 28 days, 7 to 21 days, 14 to 21 days, or 7 to 14 days before administration of one or more doses of a therapeutically effective amount of a composition disclosed herein. In some embodiments, a subject has not received an immunosuppressive medication about 7 days, about 14 days, about 21 days, about 28 days, about 35 days, about 42 days, about 49 days, or about 56 days before administration of one or more doses of a therapeutically effective amount of a composition disclosed herein. In some embodiments, a subject has not received an immunosuppressive medication about 28 days before administration of one or more doses of a therapeutically effective amount of a composition disclosed herein.

In some embodiments, an immunosuppressive medication comprises a systemic corticosteroid or radiotherapy. In some embodiments, a systemic coriticosteroid is selected from, but not limited to, methylprednisolone, dexamethasone, hydrocortisone, prednisone, prednisolone, fluticasone, flumethasone, fluocinolone, budesonide, beclomethasone, ciclesonide, cortisone, triamcinolone, betamethasone, deflazacort, difluprednate, loteprednol, paramethasone, tixocortol, aldosterone, cloprednol, cortivazol, deoxycortone, desonide, desoximetasone, difluorocortolone, fluclorolone, fludrocortisone, flunisolide, fluocinonide, fluocortin butyl, fluorocortisone, fluorocortolone, fluorometholone, flurandrenolone, halcinonide, icomethasone, meprednisone, mometasone, rofleponide, RPR 106541, and their respective pharmaceutically acceptable derivatives, such as beclomethasone dipropionate (anhydrous or monohydrate), beclomethasone monopropionate, dexamethasone 21-isonicotinate, fluticasone propionate, icomethasone enbutate, tixocortol 21-pivalate, triamcinolone acetonide, and pharmaceutically acceptable salts and/or derivatives thereof. In some embodiments, a coriticosteroid is prednisone.

In some embodiments, a subject has not received a prophylactic antipyretic and/or an analgesic medication 0 to 600 days, 0 to 550 days, 0 to 500 days, 0 to 500 days, 0 to 450 days, 0 to 400 days, 0 to 350 days, 0 to 300 days, 0 to 250 days, 0 to 200 days, 0 to 150 days, 0 to 150 days, 0 to 150 days, or 0 to 50 days before administration of one or more doses of a therapeutically effective amount of a composition disclosed herein.

In some embodiments, a prophylactic antipyretic medication is selected from, but not limited to, acetaminophen, a non-steroidal anti-inflammatory drug (NSAID), salicylamide, salicyl salicylate, methyl salicylate, magnesium salicylate, faislamine, ethenzamide, diflunisal, choline magnesium salicylate, benorylate/benorilatem and amoxiprin, acetylsalicylate, ceclofenac, acemetacin, alclofenac, bromfenac, diclofenac, etodolac, indomethacin, nabumetone, oxametacin, proglumetacin, sulindac, tolmetin, iminoprofen, benoxaprofen, carprofen, dexibuprofen, dexketoprofen, fenbufen, fenoprofen, flunoxaprofen, flurbiprofen, ibuprofen, ibuproxam, indoprofen, ketoprofen, ketorolac, loxoprofen, naproxen, oxaprozin, pirprofen, suprofen, tiaprofenic acid, mefenamic acid, flufenamic acid, meclofenamic acid, tolfenamic acid, droxicam, lornoxicam, meloxicam, piroxicam, tenoxicam, dipyrone, azapropazone, clofezone, kebuzone, metamizole, mofebutazone, oxyphenbutazone, phenazone, phenylbutazone, sulfinpyrazone, decoxib, rofecoxib, parecoxib, and etoricoxib.

In some embodiments, a prophylactic analgesic medication is selected from, but not limited to, acetaminophen, salicylamide, salicyl salicylate, methyl salicylate, magnesium salicylate, faislamine, ethenzamide, diflunisal, choline magnesium salicylate, benorylate/benorilatem and amoxiprin, acetylsalicylate, ceclofenac, acemetacin, alclofenac, bromfenac, diclofenac, etodolac, indomethacin, nabumetone, oxametacin, proglumetacin, sulindac, tolmetin, iminoprofen, benoxaprofen, carprofen, dexibuprofen, dexketoprofen, fenbufen, fenoprofen, flunoxaprofen, flurbiprofen, ibuprofen, ibuproxam, indoprofen, ketoprofen, ketorolac, loxoprofen, naproxen, oxaprozin, pirprofen, suprofen, tiaprofenic acid, mefenamic acid, flufenamic acid, meclofenamic acid, tolfenamic acid, droxicam, lornoxicam, meloxicam, piroxicam, tenoxicam, dipyrone, azapropazone, clofezone, kebuzone, metamizole, mofebutazone, oxyphenbutazone, phenazone, phenylbutazone, sulfinpyrazone, decoxib, rofecoxib, parecoxib, etoricoxib, codeine, dihydrocodeine, morphine or a morphine derivative or pharmaceutically acceptable salt thereof, diacetylmorphine, hydrocodone, hydromorphone, levorphanol, oxymorphone, alfentanil, buprenorphine, butorphanol, fentanyl, sufentanil, meperidine, methadone, nalbuphine, propoxyphene, pentazocine, and pharmaceutically acceptable salts thereof.

### VII. Treatment Methods

In some embodiments, technologies of the present disclosure may be administered to subjects according to a particular dosing regimen. In some embodiments, a dosing regimen may involve a single administration; in some embodiments, a dosing regimen may comprise one or more “booster” administrations after the initial administration. In some embodiments, initial and boost doses are the same amount; in some embodiments they differ. In some embodiments, two or more booster doses are administered. In some embodiments, a plurality of doses are administered at regular intervals. In some embodiments, periods of time between doses become longer. In some embodiments, one or more subsequent doses is administered if a particular clinical (e.g., reduction in neutralizing antibody levels) or situational (e.g., local development of a new strain) even arises or is detected.

In some embodiments, administered pharmaceutical compositions (e.g., immunogenic compositions, e.g., vaccines) comprising RNA constructs that encode HSV-2 gC, gD and/or gE constructs are administered in RNA doses of from about 0.1 μg to about 300 μg, about 0.5 μg to about 200 μg, or about 1 μg to about 100 μg, such as about 1 μg, about 3 μg, about 10 μg, about 30 μg, about 50 μg, or about 100 μg. In some embodiments, an saRNA construct is administered at a lower dose (e.g., 2, 4, 5, 10 fold or more lower) than a modRNA or uRNA construct.

In some embodiments, a first booster dose is administered about six months of the initial dose, and preferably about 5, 4, 3, 2, or 1 months. In some embodiments, a first booster dose is administered in a time period that begins about 1, 2, 3, or 4 weeks after the first dose, and ends about 2, 3, 4, 5, 6, 7, 8, 9, 10, 11, 12 weeks of the first dose (e.g., between about 1 and about 12 weeks after the first dose, or between about 2 or 3 weeks and about 5 and 6 weeks after the first dose, or about 3 weeks or about 4 weeks after the first dose).

In some embodiments, a plurality of booster doses (e.g., 2, 3, or 4) doses are administered within 6 months of the first dose, or within 12 months of the first dose.

In some embodiments, 3 doses or fewer are required to achieve effective vaccination (e.g., greater than 60%, and in some embodiments greater than about 70%, about 75%, about 80%, about 85%, about 90% or more) reduction in risk of infection, or of serious disease. In some embodiments, not more than two doses are required. In some embodiments, a single dose is sufficient. In some embodiments, an RNA dose is about 60 μg or lower, 50 μg or lower, 40 μg or lower, 30 μg or lower, 20 μg or lower, 10 μg or lower, 5 μg or lower, 2.5 μg or lower, or 1 μg or lower. In some embodiments, an RNA dose is about 0.25 μg, at least 0.5 μg, at least 1 μg, at least 2 μg, at least 3 μg, at least 4 μg, at least 5 μg, at least 10 μg, at least 20 μg, at least 30 μg, or at least 40 μg. In some embodiments, an RNA dose is about 0.25 μg to 60 μg, 0.5 μg to 55 μg, 1 μg to 50 μg, 5 μg to 40 μg, or 10 μg to 30 μg may be administered per dose. In some embodiments, an RNA dose is about 30 μg. In some embodiments, at least two such doses are administered. For example, a second dose may be administered about 21 days following administration of the first dose. In some embodiments, a first booster dose is administered about one month after an initial dose. In some such embodiments, at least one further booster is administered at one-month interval(s). In some embodiments, after 2 or 3 boosters, a longer interval is introduced and no further booster is administered for at least 6, 9, 12, 18, 24, or more months. In some embodiments, a single further booster is administered after about 18 months. In some embodiments, no further booster is required unless, for example, a material change in clinical or environmental situation is observed.

### VIII. Methods of Manufacture

Individual polyribonucleotides can be produced by methods known in the art. For example, in some embodiments, polyribonucleotides can be produced by in vitro transcription, for example, using a DNA template. A plasmid DNA used as a template for in vitro transcription to generate a polyribonucleotide described herein is also within the scope of the present disclosure.

A DNA template is used for in vitro RNA synthesis in the presence of an appropriate RNA polymerase (e.g., a recombinant RNA-polymerase such as a T7 RNA-polymerase) with ribonucleotide triphosphates (e.g., ATP, CTP, GTP, UTP). In some embodiments, polyribonucleotides (e.g., ones described herein) can be synthesized in the presence of modified ribonucleotide triphosphates. By way of example only, in some embodiments, pseudouridine (ψ), N1-methyl-pseudouridine (m1ψ), or 5-methyl-uridine (m5U) can be used to replace uridine triphosphate (UTP). In some embodiments, pseudouridine (v) can be used to replace uridine triphosphate (UTP). In some embodiments, N1-methyl-pseudouridine (ml) can be used to replace uridine triphosphate (UTP). In some embodiments, 5-methyl-uridine (m5U) can be used to replace uridine triphosphate (UTP).

As will be clear to those skilled in the art, during in vitro transcription, an RNA polymerase (e.g., as described and/or utilized herein) typically traverses at least a portion of a single-stranded DNA template in the 3′→5′ direction to produce a single-stranded complementary RNA in the 5′→3′ direction.

In some embodiments where a polyribonucleotide comprises a polyA tail, one of those skill in the art will appreciate that such a polyA tail may be encoded in a DNA template, e.g., by using an appropriately tailed PCR primer, or it can be added to a polyribonucleotide after in vitro transcription, e.g., by enzymatic treatment (e.g., using a poly(A) polymerase such as an E. coli Poly(A) polymerase). Suitable poly(A) tails are described herein above. For example, in some embodiments, a poly(A) tail comprises a nucleotide sequence of AAAAAAAAAAAAAAAAAAAAAAAAAAAAAAGCATATGACTAAAAAAAAAAAAAA AAAAAAAAAAAAAAAAAAAAAAAAAAAAAAAAAAAAAAAAAAAAAAAAAAAAAA AA (SEQ ID NO: 153). In some embodiments, a poly(A) tail comprises a plurality of A residues interrupted by a linker. In some embodiments, a linker comprises the nucleotide sequence GCATATGAC (SEQ ID NO: 154).

In some embodiments, those skilled in the art will appreciate that addition of a 5′ cap to an RNA (e.g., mRNA) can facilitate recognition and attachment of the RNA to a ribosome to initiate translation and enhances translation efficiency. Those skilled in the art will also appreciate that a 5′ cap can also protect an RNA product from 5′ exonuclease mediated degradation and thus increases half-life. Methods for capping are known in the art; one of ordinary skill in the art will appreciate that in some embodiments, capping may be performed after in vitro transcription in the presence of a capping system (e.g., an enzyme-based capping system such as, e.g., capping enzymes of vaccinia virus). In some embodiments, a cap may be introduced during in vitro transcription, along with a plurality of ribonucleotide triphosphates such that a cap is incorporated into a polyribonucleotide during transcription (also known as co-transcriptional capping). In some embodiments, a GTP fed-batch procedure with multiple additions in the course of the reaction may be used to maintain a low concentration of GTP in order to effectively cap the RNA. Suitable 5′ cap are described herein above. For example, in some embodiments, a 5′ cap comprises m7(3′OMeG)(5′)ppp(5′)(2′OMeA)pG.

Following RNA transcription, a DNA template is digested. In some embodiments, digestion can be achieved with the use of DNase I under appropriate conditions.

In some embodiments, in-vitro transcribed polyribonucleotides may be provided in a buffered solution, for example, in a buffer such as HEPES, a phosphate buffer solution, a citrate buffer solution, an acetate buffer solution; in some embodiments, such solution may be buffered to a pH within a range of, for example, about 6.5 to about 7.5; in some embodiments approximately 7.0. In some embodiments, production of polyribonucleotides may further include one or more of the following steps: purification, mixing, filtration, and/or filling.

In some embodiments, polyribonucleotides can be purified (e.g., in some embodiments after in vitro transcription reaction), for example, to remove components utilized or formed in the course of the production, like, e.g., proteins, DNA fragments, and/or or nucleotides. Various nucleic acid purifications that are known in the art can be used in accordance with the present disclosure. Certain purification steps may be or include, for example, one or more of precipitation, column chromatography (including, e.g., but not limited to anionic, cationic, hydrophobic interaction chromatography (HIC)), solid substrate-based purification (e.g., magnetic bead-based purification). In some embodiments, polyribonucleotides may be purified using magnetic bead-based purification, which in some embodiments may be or comprise magnetic bead-based chromatography. In some embodiments, polyribonucleotides may be purified using hydrophobic interaction chromatography (HIC) and/or diafiltration. In some embodiments, polyribonucleotides may be purified using HIC followed by diafiltration.

In some embodiments, dsRNA may be obtained as side product during in vitro transcription. In some such embodiments, a second purification step may be performed to remove dsRNA contamination. For example, in some embodiments, cellulose materials (e.g., microcrystalline cellulose) may be used to remove dsRNA contamination, for examples in some embodiments in a chromatographic format. In some embodiments, cellulose materials (e.g., microcrystalline cellulose) can be pretreated to inactivate potential RNase contamination, for example in some embodiments by autoclaving followed by incubation with aqueous basic solution, e.g., NaOH. In some embodiments, cellulose materials may be used to purify polyribonucleotides according to methods described in WO 2017/182524, the entire content of which is incorporated herein by reference.

In some embodiments, a batch of polyribonucleotides may be further processed by one or more steps of filtration and/or concentration. For example, in some embodiments, polyribonucleotide(s), for example, after removal of dsRNA contamination, may be further subject to diafiltration (e.g., in some embodiments by tangential flow filtration), for example, to adjust the concentration of polyribonucleotides to a desirable RNA concentration and/or to exchange buffer to a drug substance buffer.

In some embodiments, polyribonucleotides may be processed through 0.2 μm filtration before they are filled into appropriate containers.

In some embodiments, polyribonucleotides and compositions thereof may be manufactured in accordance with a process as described herein, or as otherwise known in the art.

In some embodiments, polyribonucleotides and compositions thereof may be manufactured at a large scale. For example, in some embodiments, a batch of polyribonucleotides can be manufactured at a scale of greater than 1 g, greater than 2 g, greater than 3 g, greater than 4 g, greater than 5 g, greater than 6 g, greater than 7 g, greater than 8 g, greater than 9 g, greater than 10 g, greater than 15 g, greater than 20 g, or higher.

In some embodiments, RNA quality control may be performed and/or monitored at any time during production process of polyribonucleotides and/or compositions comprising the same. For example, in some embodiments, RNA quality control parameters, including one or more of RNA identity (e.g., sequence, length, and/or RNA natures), RNA integrity, RNA concentration, residual DNA template, and residual dsRNA, may be assessed and/or monitored after each or certain steps of a polyribonucleotide manufacturing process, e.g., after in vitro transcription, and/or each purification step.

In some embodiments, the stability of polyribonucleotides (e.g., produced by in vitro transcription) and/or compositions comprising polyribonucleotides can be assessed under various test storage conditions, for example, at room temperatures vs. fridge or sub-zero temperatures over a period of time (e.g., at least 3 months, at least 6 months, at least 9 months, at least 12 months, or longer). In some embodiments, polyribonucleotides (e.g., ones described herein) and/or compositions thereof may be stored stable at a fridge temperature (e.g., about 4° C. to about 10° C.) for at least 1 month or longer including, at least 2 months, at least 3 months, at least 4 months, at least 5 months, at least 6 months, at least 7 months, at least 8 months, at least 9 months, at least 10 months, at least 11 months, or at least 12 months or longer. In some embodiments, polyribonucleotides (e.g., ones described herein) and/or compositions thereof may be stored stable at a sub-zero temperature (e.g., −20° C. or below) for at least 1 month or longer including, at least 2 months, at least 3 months, at least 4 months, at least 5 months, at least 6 months, at least 7 months, at least 8 months, at least 9 months, at least 10 months, at least 11 months, or at least 12 months or longer. In some embodiments, polyribonucleotides (e.g., ones described herein) and/or compositions thereof may be stored stable at room temperature (e.g., at about 25° C.) for at least 1 month or longer.

In some embodiments, one or more assessments may be utilized during manufacture, or other preparation or use of polyribonucleotides (e.g., as a release test).

In some embodiments, one or more quality control parameters may be assessed to determine whether polyribonucleotides described herein meet or exceed acceptance criteria (e.g., for subsequent formulation and/or release for distribution). In some embodiments, such quality control parameters may include, but are not limited to RNA integrity, RNA concentration, residual DNA template and/or residual dsRNA. Certain methods for assessing RNA quality are known in the art; for example, one of skill in the art will recognize that in some embodiments, one or more analytical tests can be used for RNA quality assessment. Examples of such certain analytical tests may include but are not limited to gel electrophoresis, UV absorption, and/or PCR assay.

In some embodiments, a batch of polyribonucleotides may be assessed for one or more features as described herein to determine next action step(s). For example, a batch of polyribonucleotides can be designated for one or more further steps of manufacturing and/or formulation and/or distribution if RNA quality assessment indicates that such a batch of polyribonucleotides meet or exceed the relevant acceptance criteria. Otherwise, an alternative action can be taken (e.g., discarding the batch) if such a batch of polyribonucleotides does not meet or exceed the acceptance criteria.

In some embodiments, a batch of polyribonucleotides that satisfy assessment results can be utilized for one or more further steps of manufacturing and/or formulation and/or distribution.

### IX. DNA Constructs

Among other things, the present disclosure provides DNA constructs, for example that may encode one or more antibody agents as described herein, or components thereof. In some embodiments, DNA constructs provided by and/or utilized in accordance with the present disclosure are comprised in a vector.

Non-limiting examples of a vector include plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). In some embodiments, a vector is an expression vector. In some embodiments, a vector is a cloning vector. In general, a vector is a nucleic acid construct that can receive or otherwise become linked to a nucleic acid element of interest (e.g., a construct that is or encodes a payload, or that imparts a particular functionality, etc.).

Expression vectors, which may be plasmid or viral or other vectors, typically include an expressible sequence of interest (e.g., a coding sequence) that is functionally linked with one or more control elements (e.g., promoters, enhancers, transcription terminators, etc.). Typically, such control elements are selected for expression in a system of interest. In some embodiments, a system is ex vivo (e.g., an in vitro transcription system); in some embodiments, a system is in vivo (e.g., a bacterial, yeast, plant, insect, fish, vertebrate, mammalian cell or tissue, etc.).

Cloning vectors are generally used to modify, engineer, and/or duplicate (e.g., by replication in vivo, for example in a simple system such as bacteria or yeast, or in vitro, such as by amplification such as polymerase chain reaction or other amplification process). In some embodiments, a cloning vector may lack expression signals.

In many embodiments, a vector may include replication elements such as primer binding site(s) and/or origin(s) of replication. In many embodiments, a vector may include insertion or modification sites such as restriction endonuclease recognition sites and/or guide RNA binding sites, etc.

In some embodiments, a vector is a viral vector (e.g., an AAV vector). In some embodiments, a vector is a non-viral vector. In some embodiments, a vector is a plasmid.

Those skilled in the art are aware of a variety of technologies useful for the production of recombinant polynucleotides (e.g., DNA or RNA) as described herein. For example, restriction digestion, reverse transcription, amplification (e.g., by polymerase chain reaction), Gibson assembly, etc., are well established and useful tools and technologies. Alternatively or additionally, certain nucleic acids may be prepared or assembled by chemical and/or enzymatic synthesis. In some embodiments, a combination of known methods is utilized to prepare a recombinant polynucleotide.

In some embodiments, polynucleotide(s) of the present disclosure are included in a DNA construct (e.g., a vector) amenable to transcription and/or translation.

In some embodiments, an expression vector comprises a polynucleotide that encodes proteins and/or polypeptides of the present disclosure operatively linked to a sequence or sequences that control expression (e.g., promoters, start signals, stop signals, polyadenylation signals, activators, repressors, etc.). In some embodiments, a sequence or sequences that control expression are selected to achieve a desired level of expression. In some embodiments, more than one sequence that controls expression (e.g., promoters) are utilized. In some embodiments, more than one sequence that controls expression (e.g., promoters) are utilized to achieve a desired level of expression of a plurality of polynucleotides that encode a plurality proteins and/or polypeptides. In some embodiments, a plurality of recombinant proteins and/or polypeptides are expressed from the same vector (e.g., a bi-cistronic vector, a tri-cistronic vector, multi-cistronic). In some embodiments, a plurality of polypeptides are expressed, each of which is expressed from a separate vector.

In some embodiments, an expression vector comprising a polynucleotide of the present disclosure is used to produce an RNA and/or protein and/or polypeptide in a host cell. In some embodiments, a host cell may be in vitro (e.g., a cell line)—for example a cell or cell line (e.g., Human Embryonic Kidney (HEK cells), Chinese Hamster Ovary cells, etc.) suitable for producing polynucleotides of the present disclosure and proteins and/or polypeptides encoded by said polynucleotides.

In some embodiments, an expression vector is an RNA expression vector. In some embodiments, an RNA expression vector comprises a polynucleotide template used to produce an RNA in cell-free enzymatic mix. In some embodiments, an RNA expression vector comprising a polynucleotide template is enzymatically linearized prior to in vitro transcription. In some embodiments, a polynucleotide template is generated through PCR as a linear polynucleotide template. In some embodiments, a linearized polynucleotide is mixed with enzymes suitable for RNA synthesis, RNA capping and/or purification. In some embodiments, the resulting RNA is suitable for producing proteins encoded by the RNA.

A variety of methods are known in the art to introduce an expression vector into host cells. In some embodiments, a vector may be introduced into host cells using transfection. In some embodiments, transfection is completed, for example, using calcium phosphate transfection, lipofection, or polyethylenimine-mediated transfection. In some embodiments, a vector may be introduced into a host cell using transduction.

In some embodiments, transformed host cells are cultured following introduction of a vector into a host cell to allow for expression of said recombinant polynucleotides. In some embodiments, a transformed host cells are cultured for at least 12 hours, 16 hours, 20 hours, 24 hours, 28 hours, 32 hours, 36 hours 40 hours, 44 hours, 48 hours, 52 hours, 56 hours, 60 hours, 64 hours, 68 hours, 72 hours or longer. Transformed host cells are cultured in growth conditions (e.g., temperature, carbon-dioxide levels, growth medium) in accordance with the requirements of a host cell selected. A skilled artisan would recognize culture conditions for host cells selected are well known in the art.

### X. Dosage Regimens

In some embodiments, the present disclosure provides a method of treating or preventing herpes simplex virus (HSV) infection comprising administering to a subject in need thereof a therapeutically effective amount of a composition disclosed herein, in a treatment cycle comprising one or more doses (e.g., one dose, two doses, three doses, four doses, five doses, six doses, seven doses, eight doses, nine doses, or ten doses) of the composition. In some embodiments, a treatment cycle comprises two or more doses (e.g., two doses, three doses, four doses, five doses, six doses, seven doses, eight doses, nine doses, or ten doses) of the composition. In some embodiments, a treatment cycle comprises two doses. In some embodiments, a first dose is a priming dose of a composition disclosed herein. In some embodiments, a second dose is a booster dose of a composition disclosed herein.

In some embodiments, a subject is administered one or more doses of a composition disclosed herein prior to infection with HSV (e.g., HSV-1, HSV-2, or a combination thereof). In some embodiments, a subject is administered two or more doses of a composition disclosed herein prior to infection with HSV (e.g., HSV-1, HSV-2, or a combination thereof). In some embodiments, a subject is administered three or more doses of a composition disclosed herein prior to infection with HSV (e.g., HSV-1, HSV-2, or a combination thereof).

In some embodiments, a second dose of the therapeutically effective amount of a composition disclosed herein is administered to a subject 1 day to 24 weeks, 3.5 days to 24 weeks, 1 week to 24 weeks, 2 weeks to 24 weeks, 4 weeks to 24 weeks, 6 weeks to 24 weeks, 8 weeks to 24 weeks, 10 weeks to 24 weeks, 12 weeks to 24 weeks, 16 weeks to 24 weeks, 20 weeks to 24 weeks, 1 day to 20 weeks, 3.5 days to 20 weeks, 1 week to 20 weeks, 2 weeks to 20 weeks, 4 weeks to 20 weeks, 6 weeks to 20 weeks, 8 weeks to 20 weeks, 10 weeks to 20 weeks, 12 weeks to 20 weeks, 16 weeks to 20 weeks, 1 day to 16 weeks, 3.5 days to 16 weeks, 1 week to 16 weeks, 2 weeks to 16 weeks, 4 weeks to 16 weeks, 6 weeks to 16 weeks, 8 weeks to 16 weeks, 10 weeks to 16 weeks, 12 weeks to 16 weeks, 1 day to 12 weeks, 3.5 days to 12 weeks, 1 week to 12 weeks, 2 weeks to 12 weeks, 4 weeks to 12 weeks, 6 weeks to 12 weeks, 8 weeks to 12 weeks, 10 weeks to 12 weeks, 1 day to 10 weeks, 3.5 days to 10 weeks, 1 week to 10 weeks, 2 week to 10 weeks, 4 weeks to 10 weeks, 6 weeks to 10 weeks, 8 weeks to 10 weeks, 1 day to 8 weeks, 3.5 days to 8 weeks, 1 week to 8 weeks, 2 weeks to 8 weeks, 4 weeks to 8 weeks, 6 weeks to 8 weeks, 1 day to 6 weeks, 3.5 days to 6 weeks, 1 week to 6 weeks, 2 weeks to 6 weeks, 4 weeks to 6 weeks, 1 day to 4 weeks, 3.5 days to 4 weeks, 1 week to 4 weeks, 2 week to 4 weeks, 1 day to 2 weeks, 3.5 days to 2 weeks, 1 week to 2 weeks, 1 day to 1 week, 3.5 days to 1 week, or 1 day to 3.5 days, after administration of a first dose of the therapeutically effective amount of the composition to the subject. In some embodiments, a second dose of the therapeutically effective amount of a composition disclosed herein is administered 1 week to 14 weeks after administration of the first dose of the therapeutically effective amount of the composition to the subject. In some embodiments, a second dose of the therapeutically effective amount of a composition disclosed herein is administered 4 weeks to 12 weeks after administration of the first dose of the therapeutically effective amount of the composition to the subject. In some embodiments, a second dose of the therapeutically effective amount of a composition disclosed herein is administered 6 weeks to 10 weeks after administration of the first dose of the therapeutically effective amount of the composition to the subject.

In some embodiments, a second dose of the therapeutically effective amount of a composition disclosed herein is administered about 1 week, about 2 weeks, about 4 weeks, about 6 weeks, about 8 weeks, about 10 weeks, about 12 weeks, about 16 weeks, about 20 weeks, or about 24 weeks after administration of the first dose of the therapeutically effective amount of the composition to the subject. In some embodiments, a second dose of the therapeutically effective amount of a composition disclosed herein is administered about 8 weeks after administration of the first dose of the therapeutically effective amount of the composition to the subject.

In some embodiments, a second dose of the therapeutically effective amount of a composition disclosed herein is administered about 1 day, about 7 days, about 14 days, about 28 days, about 35 days, about 40 days, about 45 days, about 50 days, about 51 days, about 52 days, about 53 days, about 54 days, about 55 days, about 56 days, about 57 days, about 58 days, about 59 days, or about 60 days after administration of the first dose of the therapeutically effective amount of the composition to the subject.

In some embodiments, a third dose of the therapeutically effective amount of a composition disclosed herein is administered to a subject 1 day to 24 weeks, 3.5 days to 24 weeks, 1 week to 24 weeks, 2 weeks to 24 weeks, 4 weeks to 24 weeks, 6 weeks to 24 weeks, 8 weeks to 24 weeks, 10 weeks to 24 weeks, 12 weeks to 24 weeks, 16 weeks to 24 weeks, 20 weeks to 24 weeks, 1 day to 20 weeks, 3.5 days to 20 weeks, 1 week to 20 weeks, 2 weeks to 20 weeks, 4 weeks to 20 weeks, 6 weeks to 20 weeks, 8 weeks to 20 weeks, 10 weeks to 20 weeks, 12 weeks to 20 weeks, 16 weeks to 20 weeks, 1 day to 16 weeks, 3.5 days to 16 weeks, 1 week to 16 weeks, 2 weeks to 16 weeks, 4 weeks to 16 weeks, 6 weeks to 16 weeks, 8 weeks to 16 weeks, 10 weeks to 16 weeks, 12 weeks to 16 weeks, 1 day to 12 weeks, 3.5 days to 12 weeks, 1 week to 12 weeks, 2 weeks to 12 weeks, 4 weeks to 12 weeks, 6 weeks to 12 weeks, 8 weeks to 12 weeks, 10 weeks to 12 weeks, 1 day to 10 weeks, 3.5 days to 10 weeks, 1 week to 10 weeks, 2 week to 10 weeks, 4 weeks to 10 weeks, 6 weeks to 10 weeks, 8 weeks to 10 weeks, 1 day to 8 weeks, 3.5 days to 8 weeks, 1 week to 8 weeks, 2 weeks to 8 weeks, 4 weeks to 8 weeks, 6 weeks to 8 weeks, 1 day to 6 weeks, 3.5 days to 6 weeks, 1 week to 6 weeks, 2 weeks to 6 weeks, 4 weeks to 6 weeks, 1 day to 4 weeks, 3.5 days to 4 weeks, 1 week to 4 weeks, 2 week to 4 weeks, 1 day to 2 weeks, 3.5 days to 2 weeks, 1 week to 2 weeks, 1 day to 1 week, 3.5 days to 1 week, or 1 day to 3.5 days, after administration of a first dose of the therapeutically effective amount of the composition to the subject. In some embodiments, a third dose of the therapeutically effective amount of a composition disclosed herein is administered 4 weeks to 24 weeks after administration of the first dose of the therapeutically effective amount of the composition to the subject. In some embodiments, a third dose of the therapeutically effective amount of a composition disclosed herein is administered 12 weeks to 20 weeks after administration of the first dose of the therapeutically effective amount of the composition to the subject. In some embodiments, a third dose of the therapeutically effective amount of a composition disclosed herein is administered 14 weeks to 18 weeks after administration of the first dose of the therapeutically effective amount of the composition to the subject.

In some embodiments, a third dose of the therapeutically effective amount of a composition disclosed herein is administered about 6 weeks, about 8 weeks, about 10 weeks, about 12 weeks, about 13 weeks, about 14 weeks, about 15 weeks, about 16 weeks, about 17 weeks, about 18 weeks, about 19 weeks, about 20 weeks, or about 24 weeks after administration of the first dose of the therapeutically effective amount of the composition to the subject. In some embodiments, a third dose of the therapeutically effective amount of a composition disclosed herein is administered about 8 weeks after administration of the first dose of the therapeutically effective amount of the composition to the subject.

In some embodiments, a third dose of the therapeutically effective amount of a composition disclosed herein is administered about 10 days, about 20 days, about 30 days, about 40 days, about 50 days, about 60 days, about 70 days, about 80 days, about 90 days, about 100 days, about 105 days, about 110 days, about 111 days, about 112 days, about 113 days, about 114 days, about 115 days, about 116 days, or about 117 days after administration of the first dose of the therapeutically effective amount of the composition to the subject.

In some embodiments, each of the one or more doses of a therapeutically effective amount of a composition disclosed herein is administered to a subject intramuscularly, subcutaneously, orally, or intranasally. In some embodiments, each of the one or more doses of a therapeutically effective amount of a composition disclosed herein is administered to a subject intramuscularly.

In some embodiments, each of the one or more doses comprises 0.1 to 500 μg, 0.2 to 500 μg, 0.5 to 500 μg, 0.75 to 500 μg, 1 to 500 μg. 2.5 to 500 μg, 5 to 500 μg, 7.5 to 500 μg, 10 to 500 μg, 12.5 to 500 15 to 500 μg, 17.5 to 500 μg, 20 to 500 μg, 25 to 500 μg, 30 to 500 μg, 35 to 500 μg, 40 to 500 μg, 45 to 500 μg, 50 to 500 μg, 55 to 500 μg, 60 to 500 μg, 65 to 500 μg, 70 to 500 μg, 75 to 500 μg, 80 to 500 μg, 85 to 500 μg, 90 to 500 μg, 95 to 500 μg, 100 to 500 μg, 125 to 500 μg, 150 to 500 μg, 175 to 500 μg, 200 to 500 μg, 225 to 500 μg, 250 to 500 μg, 275 to 500 μg, 300 to 500 μg, 325 to 500 μg, 350 to 500 μg, 375 to 500 μg, 400 to 500 μg, 425 to 500 μg, 450 to 500 μg, 475 to 500 μg, 0.1 to 450 μg, 0.2 to 450 μg, 0.5 to 450 μg, 0.75 to 450 μg, 1 to 450 μg. 2.5 to 450 μg, 5 to 450 μg, 7.5 to 450 μg, 10 to 450 μg, 12.5 to 450 15 to 450 μg, 17.5 to 450 μg, 20 to 450 μg, 25 to 450 μg, 30 to 450 μg, 35 to 450 μg, 40 to 450 μg, 45 to 450 μg, 50 to 450 μg, 55 to 450 μg, 60 to 450 μg, 65 to 450 μg, 70 to 450 μg, 75 to 450 μg, 80 to 450 μg, 85 to 450 μg, 90 to 450 μg, 95 to 450 μg, 100 to 450 μg, 125 to 450 μg, 150 to 450 μg, 175 to 450 μg, 200 to 450 μg, 225 to 450 μg, 250 to 450 μg, 275 to 450 μg, 300 to 450 μg, 325 to 450 μg, 350 to 450 μg, 375 to 450 μg, 400 to 450 μg, 425 to 450 μg, 0.1 to 400 μg, 0.2 to 400 μg, 0.5 to 400 μg, 0.75 to 400 μg, 1 to 400 μg. 2.5 to 400 μg, 5 to 400 μg, 7.5 to 400 μg, 10 to 400 μg, 12.5 to 400 15 to 400 μg, 17.5 to 400 μg, 20 to 400 μg, 25 to 400 μg, 30 to 400 μg, 35 to 400 μg, 40 to 400 μg, 45 to 400 μg, 50 to 400 μg, 55 to 400 μg, 60 to 400 μg, 65 to 400 μg, 70 to 400 μg, 75 to 400 μg, 80 to 400 μg, 85 to 400 μg, 90 to 400 μg, 95 to 400 μg, 100 to 400 μg, 125 to 400 μg, 150 to 400 μg, 175 to 400 μg, 200 to 400 μg, 225 to 400 μg, 250 to 400 μg, 275 to 400 μg, 300 to 400 μg, 325 to 400 μg, 350 to 400 μg, 375 to 400 μg, 0.1 to 350 μg, 0.2 to 350 μg, 0.5 to 350 μg, 0.75 to 350 μg, 1 to 350 μg. 2.5 to 350 μg, 5 to 350 μg, 7.5 to 350 μg, 10 to 350 μg, 12.5 to 350 15 to 350 μg, 17.5 to 350 μg, 20 to 350 μg, 25 to 350 μg, 30 to 350 μg, 35 to 350 μg, 40 to 350 μg, 45 to 350 μg, 50 to 350 μg, 55 to 350 μg, 60 to 350 μg, 65 to 350 μg, 70 to 350 μg, 75 to 350 μg, 80 to 350 μg, 85 to 350 μg, 90 to 350 μg, 95 to 350 μg, 100 to 350 μg, 125 to 350 μg, 150 to 350 μg, 175 to 350 μg, 200 to 350 μg, 225 to 350 μg, 250 to 350 μg, 275 to 350 μg, 300 to 350 μg, 325 to 350 μg, 0.1 to 300 μg, 0.2 to 300 μg, 0.5 to 300 μg, 0.75 to 300 μg, 1 to 300 μg. 2.5 to 300 μg, 5 to 300 μg, 7.5 to 300 μg, 10 to 300 μg, 12.5 to 300 15 to 300 μg, 17.5 to 300 μg, 20 to 300 μg, 25 to 300 μg, 30 to 300 μg, 35 to 300 μg, 40 to 300 μg, 45 to 300 μg, 50 to 300 μg, 55 to 300 μg, 60 to 300 μg, 65 to 300 μg, 70 to 300 μg, 75 to 300 μg, 80 to 300 μg, 85 to 300 μg, 90 to 300 μg, 95 to 300 μg, 100 to 300 μg, 125 to 300 μg, 150 to 300 μg, 175 to 300 μg, 200 to 300 μg, 225 to 300 μg, 250 to 300 μg, 275 to 300 μg, 0.1 to 250 μg, 0.2 to 250 μg, 0.5 to 250 μg, 0.75 to 250 μg, 1 to 250 μg. 2.5 to 250 μg, 5 to 250 μg, 7.5 to 250 μg, 10 to 250 μg, 12.5 to 250 15 to 250 μg, 17.5 to 250 μg, 20 to 250 μg, 25 to 250 μg, 30 to 250 μg, 35 to 250 μg, 40 to 250 μg, 45 to 250 μg, 50 to 250 μg, 55 to 250 μg, 60 to 250 μg, 65 to 250 μg, 70 to 250 μg, 75 to 250 μg, 80 to 250 μg, 85 to 250 μg, 90 to 250 μg, 95 to 250 μg, 100 to 250 μg, 125 to 250 μg, 150 to 250 μg, 175 to 250 μg, 200 to 250 μg, 225 to 250 μg, 0.1 to 200 μg, 0.2 to 200 μg, 0.5 to 200 μg, 0.75 to 200 μg, 1 to 200 μg. 2.5 to 200 μg, 5 to 200 μg, 7.5 to 200 μg, 10 to 200 μg, 12.5 to 200 15 to 200 μg, 17.5 to 200 μg, 20 to 200 μg, 25 to 200 μg, 30 to 200 μg, 35 to 200 μg, 40 to 200 μg, 45 to 200 μg, 50 to 200 μg, 55 to 200 μg, 60 to 200 μg, 65 to 200 μg, 70 to 200 μg, 75 to 200 μg, 80 to 200 μg, 85 to 200 μg, 90 to 200 μg, 95 to 200 μg, 100 to 200 μg, 125 to 200 μg, 150 to 200 μg, 175 to 200 μg, 0.1 to 150 μg, 0.2 to 150 μg, 0.5 to 150 μg, 0.75 to 150 μg, 1 to 150 μg. 2.5 to 150 μg, 5 to 150 μg, 7.5 to 150 μg, 10 to 150 μg, 12.5 to 150 15 to 150 μg, 17.5 to 150 μg, 20 to 150 μg, 25 to 150 μg, 30 to 150 μg, 35 to 150 μg, 40 to 150 μg, 45 to 150 μg, 50 to 150 μg, 55 to 150 μg, 60 to 150 μg, 65 to 150 μg, 70 to 150 μg, 75 to 150 μg, 80 to 150 μg, 85 to 150 μg, 90 to 150 μg, 95 to 150 μg, 100 to 150 μg, 125 to 150 μg, 0.1 to 100 μg, 0.2 to 100 μg, 0.5 to 100 μg, 0.75 to 100 μg, 1 to 100 μg. 2.5 to 100 μg, 5 to 100 μg, 7.5 to 100 μg, 10 to 100 μg, 12.5 to 100 15 to 100 μg, 17.5 to 100 μg, 20 to 100 μg, 25 to 100 μg, 30 to 100 μg, 35 to 100 μg, 40 to 100 μg, 45 to 100 μg, 50 to 100 μg, 55 to 100 μg, 60 to 100 μg, 65 to 100 μg, 70 to 100 μg, 75 to 100 μg, 80 to 100 μg, 85 to 100 μg, 90 to 100 μg, 95 to 100 μg, 0.1 to 90 μg, 0.2 to 90 μg, 0.5 to 90 μg, 0.75 to 90 μg, 1 to 90 μg. 2.5 to 90 μg, 5 to 90 μg, 7.5 to 90 μg, 10 to 90 μg, 12.5 to 90 15 to 90 μg, 17.5 to 90 μg, 20 to 90 μg, 25 to 90 μg, 30 to 90 μg, 35 to 90 μg, 40 to 90 μg, 45 to 90 μg, 50 to 90 μg, 55 to 90 μg, 60 to 90 μg, 65 to 90 μg, 70 to 90 μg, 75 to 90 μg, 80 to 90 μg, 85 to 90 μg, 0.1 to 80 μg, 0.2 to 80 μg, 0.5 to 80 μg, 0.75 to 80 μg, 1 to 80 μg. 2.5 to 80 μg, 5 to 80 μg, 7.5 to 80 μg, 10 to 80 μg, 12.5 to 80 μg, 15 to 80 μg, 17.5 to 80 μg, 20 to 80 μg, 25 to 80 μg, 30 to 80 μg, 35 to 80 μg, 40 to 80 μg, 45 to 80 μg, 50 to 80 μg, 55 to 80 μg, 60 to 80 μg, 65 to 80 μg, 70 to 80 μg, 75 to 80 μg, 0.1 to 70 μg, 0.2 to 70 μg, 0.5 to 70 μg, 0.75 to 70 μg, 1 to 70 μg. 2.5 to 70 μg, 5 to 70 μg, 7.5 to 70 μg, 10 to 70 μg, 12.5 to 70 μg, 15 to 70 μg, 17.5 to 70 μg, 20 to 70 μg, 25 to 70 μg, 30 to 70 μg, 35 to 70 μg, 40 to 70 μg, 45 to 70 μg, 50 to 70 μg, 55 to 70 μg, 60 to 70 μg, 65 to 70 μg, 0.1 to 60 μg, 0.2 to 60 μg, 0.5 to 60 μg, 0.75 to 60 μg, 1 to 60 μg. 2.5 to 60 μg, 5 to 60 μg, 7.5 to 60 μg, 10 to 60 μg, 12.5 to 60 μg, 15 to 60 μg, 17.5 to 60 μg, 20 to 60 μg, 25 to 60 μg, 30 to 60 μg, 35 to 60 μg, 40 to 60 μg, 45 to 60 μg, 50 to 60 μg, 55 to 60 μg, 0.1 to 50 μg, 0.2 to 50 μg, 0.5 to 50 μg, 0.75 to 50 μg, 1 to 50 μg. 2.5 to 50 μg, 5 to 50 μg, 7.5 to 50 μg, 10 to 50 μg, 12.5 to 50 μg, 15 to 50 μg, 17.5 to 50 μg, 20 to 50 μg, 25 to 50 μg, 30 to 50 μg, 35 to 50 μg, 40 to 50 μg, 45 to 50 μg, 0.1 to 40 μg, 0.2 to 40 μg, 0.5 to 40 μg, 0.75 to 40 μg, 1 to 40 μg. 2.5 to 40 μg, 5 to 40 μg, 7.5 to 40 μg, 10 to 40 μg, 12.5 to 40 μg, 15 to 40 μg, 17.5 to 40 μg, 20 to 40 μg, 25 to 40 μg, 30 to 40 μg, 35 to 40 μg, 0.1 to 30 μg, 0.2 to 30 μg, 0.5 to 30 μg, 0.75 to 30 μg, 1 to 30 μg. 2.5 to 30 μg, 5 to 30 μg, 7.5 to 30 μg, 10 to 30 μg, 12.5 to 30 15 to 30 μg, 17.5 to 30 μg, 20 to 30 μg, 25 to 30 μg, 0.1 to 20 μg, 0.2 to 20 μg, 0.5 to 20 μg, 0.75 to 20 μg, 1 to 20 μg. 2.5 to 20 μg, 5 to 20 μg, 7.5 to 20 μg, 10 to 20 μg, 12.5 to 20 15 to 20 μg, 17.5 to 20 μg, 0.1 to 10 μg, 0.2 to 10 μg, 0.5 to 10 μg, 0.75 to 10 μg, 1 to 10 μg. 2.5 to 10 μg, 5 to 10 μg, 7.5 to 10 μg, 0.1 to 1 μg, 0.2 to 1 μg, 0.5 to 1 μg, or 0.75 to 1 μg of one or more polyribonucleotides encoding one or more HSV glycoprotein antigens or antigenic fragments thereof.

In some embodiments, each of the one or more doses comprises 1 μg to 250 μg of one or more polyribonucleotides encoding one or more HSV glycoproteins or antigenic fragments thereof. In some embodiments, each of the one or more doses comprises 2 μg to 200 μg of one or more polyribonucleotides encoding one or more HSV glycoproteins or antigenic fragments thereof. In some embodiments, each of the one or more doses comprises 3 μg to 100 μg of one or more polyribonucleotides encoding one or more HSV glycoproteins or antigenic fragments thereof. In some embodiments, each of the one or more doses comprises 3 μg to 60 μg of one or more polyribonucleotides encoding one or more HSV glycoproteins or antigenic fragments thereof. In some embodiments, each of the one or more doses comprises 3 μg of one or more polyribonucleotides encoding one or more HSV glycoproteins or antigenic fragments thereof. In some embodiments, each of the one or more doses comprises 10 μg of one or more polyribonucleotides encoding one or more HSV glycoproteins or antigenic fragments thereof. In some embodiments, each of the one or more doses comprises 30 μg of one or more polyribonucleotides encoding one or more HSV glycoproteins or antigenic fragments thereof. In some embodiments, each of the one or more doses comprises 60 μg of one or more polyribonucleotides encoding one or more HSV glycoproteins or antigenic fragments thereof.

In some embodiments, each of the one or more doses comprises 0.5 μg, 0.75 μg, 1.0 μg, 1.25 μg, 1.33 μg, 1.5 μg, 1.75 μg, 2.0 μg, 2.25 μg, 2.5 μg, 5 μg, 7.5 μg, 10 μg, 15 μg, 20 μg, or 25 μg of one or more polyribonucleotides encoding one or more HSV glycoproteins or antigenic fragments thereof. For example, in some embodiments, each of the one or more doses comprises 1 μg of each of three polyribonucleotides, wherein each polyribonucleotide encodes a different HSV glycoprotein or antigenic fragment thereof. In another example, in some embodiments, each of the one or more doses comprises 3.33 μg of each of three polyribonucleotides, wherein each polyribonucleotide encodes a different HSV glycoprotein or antigenic fragment thereof. In yet another example, in some embodiments, each of the one or more doses comprises 10 μg of each of three polyribonucleotides, wherein each polyribonucleotide encodes a different HSV glycoprotein or antigenic fragment thereof. In some embodiments, each of the one or more doses comprises 20 μg of each of three polyribonucleotides, wherein each polyribonucleotide encodes a different HSV glycoprotein or antigenic fragment thereof.

### XI. Concomitant Therapies

Combinatorial Use with Antipyretic and/or Analgesic Medications

In some embodiments, the present disclosure provides a method of treating or preventing HSV infection comprising administering to a subject in need thereof one or more doses of a therapeutically effective amount of a composition as disclosed herein concomitantly with administration of an antipyretic medication. In some embodiments, an antipyretic medication is administered within 60 minutes, within 30 minutes, or within 15 minutes after administration of one or more doses of a therapeutically effective amount of a composition as disclosed herein. In some embodiments, an antipyretic medication is administered concurrently with administration of one or more doses of a therapeutically effective amount of a composition as disclosed herein. In some embodiments, an antipyretic medication is administered within 60 minutes, within 30 minutes, or within 15 minutes before administration of one of more doses of a therapeutically effective amount of a composition as disclosed herein.

In some embodiments, the antipyretic is acetaminophen, a non-steroidal anti-inflammatory drug (NSAID), salicylamide, salicyl salicylate, methyl salicylate, magnesium salicylate, faislamine, ethenzamide, diflunisal, choline magnesium salicylate, benorylate/benorilatem and amoxiprin, acetylsalicylate, ceclofenac, acemetacin, alclofenac, bromfenac, diclofenac, etodolac, indomethacin, nabumetone, oxametacin, proglumetacin, sulindac, tolmetin, iminoprofen, benoxaprofen, carprofen, dexibuprofen, dexketoprofen, fenbufen, fenoprofen, flunoxaprofen, flurbiprofen, ibuprofen, ibuproxam, indoprofen, ketoprofen, ketorolac, loxoprofen, naproxen, oxaprozin, pirprofen, suprofen, tiaprofenic acid, mefenamic acid, flufenamic acid, meclofenamic acid, tolfenamic acid, droxicam, lornoxicam, meloxicam, piroxicam, tenoxicam, dipyrone, azapropazone, clofezone, kebuzone, metamizole, mofebutazone, oxyphenbutazone, phenazone, phenylbutazone, sulfinpyrazone, decoxib, rofecoxib, parecoxib, and etoricoxib. In certain embodiments, the antipyretic is an NSAID. In certain embodiments, the antipyretic is acetaminophen.

In some embodiments, the present disclosure provides a method of treating or preventing HSV (HSV-1, HSV-2, or a combination thereof) infection comprising administering to a subject in need thereof one or more doses of a therapeutically effective amount of a composition as disclosed herein concomitantly with administration of an analgesic medication. In some embodiments, an analgesic medication is administered within 60 minutes, within 30 minutes, or within 15 minutes after administration of one or more doses of a therapeutically effective amount of a composition as disclosed herein. In some embodiments, an analgesic medication is administered concurrently with administration of one or more doses of a therapeutically effective amount of a composition as disclosed herein. In some embodiments, an analgesic medication is administered within 60 minutes, within 30 minutes, or within 15 minutes before administration of one of more doses of a therapeutically effective amount of a composition as disclosed herein.

In some embodiments, an analgesic is acetaminophen, salicylamide, salicyl salicylate, methyl salicylate, magnesium salicylate, faislamine, ethenzamide, diflunisal, choline magnesium salicylate, benorylate/benorilatem and amoxiprin, acetylsalicylate, ceclofenac, acemetacin, alclofenac, bromfenac, diclofenac, etodolac, indomethacin, nabumetone, oxametacin, proglumetacin, sulindac, tolmetin, iminoprofen, benoxaprofen, carprofen, dexibuprofen, dexketoprofen, fenbufen, fenoprofen, flunoxaprofen, flurbiprofen, ibuprofen, ibuproxam, indoprofen, ketoprofen, ketorolac, loxoprofen, naproxen, oxaprozin, pirprofen, suprofen, tiaprofenic acid, mefenamic acid, flufenamic acid, meclofenamic acid, tolfenamic acid, droxicam, lornoxicam, meloxicam, piroxicam, tenoxicam, dipyrone, azapropazone, clofezone, kebuzone, metamizole, mofebutazone, oxyphenbutazone, phenazone, phenylbutazone, sulfinpyrazone, decoxib, rofecoxib, parecoxib, etoricoxib, codeine, dihydrocodeine, morphine or a morphine derivative or pharmaceutically acceptable salt thereof, diacetylmorphine, hydrocodone, hydromorphone, levorphanol, oxymorphone, alfentanil, buprenorphine, butorphanol, fentanyl, sufentanil, meperidine, methadone, nalbuphine, propoxyphene, pentazocine, or pharmaceutically acceptable salts thereof. In some embodiments, the analgesic is acetaminophen.

In some embodiments, the present disclosure provides a method of treating or preventing HSV (HSV-1, HSV-2, or a combination thereof) infection comprising administering to a subject in need thereof one or more doses of a therapeutically effective amount of a composition as disclosed herein concomitantly with administration of an antipyretic medication and an analgesic medication. In some embodiments, an antipyretic medication and analgesic medication is administered within 60 minutes, within 30 minutes, or within 15 minutes after administration of one or more doses of a therapeutically effective amount of a composition as disclosed herein. In some embodiments, an antipyretic medication and analgesic medication is administered concurrently with administration of one or more doses of a therapeutically effective amount of a composition as disclosed herein. In some embodiments, an antipyretic medication and analgesic medication is administered within 60 minutes, within 30 minutes, or within 15 minutes before administration of one of more doses of a therapeutically effective amount of a composition as disclosed herein.

In some embodiments, an antipyretic medication and/or analgesic medication is acetaminophen. In some embodiments, acetaminophen is administered to the subject at a dose of 0.1 g/day to 20 g/day, 0.25 g/day to 20 g/day, 0.5 g/day to 20 g/day, 0.75 g/day to 20 g/day, 1.0 g/day to 20 g/day, 1.25 g/day to 20 g/day, 1.5 g/day to 20 g/day, 1.75 g/day to 20 g/day, 2.0 g/day to 20 g/day, 2.25 g/day to 201 g/day, 2.5 g/day to 20 g/day, 2.75 g/day to 20 g/day, 3.0 g/day to 20 g/day, 3.25 g/day to 20 g/day, 3.5 g/day to 20 g/day, 3.75 g/day to 20 g/day, 4.0 g/day to 20 g/day, 4.25 g/day to 20 g/day, 4.5 g/day to 20 g/day, 4.75 g/day to 20 g/day, 5.0 g/day to 20 g/day, 5.25 g/day to 20 g/day, 5.5 g/day to 20 g/day, 5.75 g/day to 20 g/day, 6.0 g/day to 20 g/day, 6.25 g/day to 20 g/day, 6.5 g/day to 20 g/day, 6.75 g/day to 20 g/day, 7.0 g/day to 20 g/day, 7.25 g/day to 20 g/day, 7.5 g/day to 20 g/day, 7.75 g/day to 20 g/day, 8.0 g/day to 20 g/day, 8.25 g/day to 20 g/day, 8.5 g/day to 20 g/day, 8.75 g/day to 20 g/day, 9.0 g/day to 20 g/day, 9.25 g/day to 20 g/day, 9.5 g/day to 20 g/day, 9.75 g/day to 20 g/day, 10 g/day to 20 g/day, 12.5 g/day to 20 g/day, 15 g/day to 20 g/day, 17.5 g/day to 20 g/day, 0.1 g/day to 15 g/day, 0.25 g/day to 15 g/day, 0.5 g/day to 15 g/day, 0.75 g/day to 15 g/day, 1.0 g/day to 15 g/day, 1.25 g/day to 15 g/day, 1.5 g/day to 15 g/day, 1.75 g/day to 15 g/day, 2.0 g/day to 15 g/day, 2.25 g/day to 151 g/day, 2.5 g/day to 15 g/day, 2.75 g/day to 15 g/day, 3.0 g/day to 15 g/day, 3.25 g/day to 15 g/day, 3.5 g/day to 15 g/day, 3.75 g/day to 15 g/day, 4.0 g/day to 15 g/day, 4.25 g/day to 15 g/day, 4.5 g/day to 15 g/day, 4.75 g/day to 15 g/day, 5.0 g/day to 15 g/day, 5.25 g/day to 15 g/day, 5.5 g/day to 15 g/day, 5.75 g/day to 15 g/day, 6.0 g/day to 15 g/day, 6.25 g/day to 15 g/day, 6.5 g/day to 15 g/day, 6.75 g/day to 15 g/day, 7.0 g/day to 15 g/day, 7.25 g/day to 15 g/day, 7.5 g/day to 15 g/day, 7.75 g/day to 15 g/day, 8.0 g/day to 15 g/day, 8.25 g/day to 15 g/day, 8.5 g/day to 15 g/day, 8.75 g/day to 15 g/day, 9.0 g/day to 15 g/day, 9.25 g/day to 15 g/day, 9.5 g/day to 15 g/day, 9.75 g/day to 15 g/day, 10 g/day to 15 g/day, 12.5 g/day to 15 g/day, 0.1 g/day to 10 g/day, 0.25 g/day to 10 g/day, 0.5 g/day to 10 g/day, 0.75 g/day to 10 g/day, 1.0 g/day to 10 g/day, 1.25 g/day to 10 g/day, 1.5 g/day to 10 g/day, 1.75 g/day to 10 g/day, 2.0 g/day to 10 g/day, 2.25 g/day to 101 g/day, 2.5 g/day to 10 g/day, 2.75 g/day to 10 g/day, 3.0 g/day to 10 g/day, 3.25 g/day to 10 g/day, 3.5 g/day to 10 g/day, 3.75 g/day to 10 g/day, 4.0 g/day to 10 g/day, 4.25 g/day to 10 g/day, 4.5 g/day to 10 g/day, 4.75 g/day to 10 g/day, 5.0 g/day to 10 g/day, 5.25 g/day to 10 g/day, 5.5 g/day to 10 g/day, 5.75 g/day to 10 g/day, 6.0 g/day to 10 g/day, 6.25 g/day to 10 g/day, 6.5 g/day to 10 g/day, 6.75 g/day to 10 g/day, 7.0 g/day to 10 g/day, 7.25 g/day to 10 g/day, 7.5 g/day to 10 g/day, 7.75 g/day to 10 g/day, 8.0 g/day to 10 g/day, 8.25 g/day to 10 g/day, 8.5 g/day to 10 g/day, 8.75 g/day to 10 g/day, 9.0 g/day to 10 g/day, 9.25 g/day to 10 g/day, 9.5 g/day to 10 g/day, 9.75 g/day to 10 g/day, 0.1 g/day to 7.5 g/day, 0.25 g/day to 7.5 g/day, 0.5 g/day to 7.5 g/day, 0.75 g/day to 7.5 g/day, 1.0 g/day to 7.5 g/day, 1.25 g/day to 7.5 g/day, 1.5 g/day to 7.5 g/day, 1.75 g/day to 7.5 g/day, 2.0 g/day to 7.5 g/day, 2.25 g/day to 7.51 g/day, 2.5 g/day to 7.5 g/day, 2.75 g/day to 7.5 g/day, 3.0 g/day to 7.5 g/day, 3.25 g/day to 7.5 g/day, 3.5 g/day to 7.5 g/day, 3.75 g/day to 7.5 g/day, 4.0 g/day to 7.5 g/day, 4.25 g/day to 7.5 g/day, 4.5 g/day to 7.5 g/day, 4.75 g/day to 7.5 g/day, 5.0 g/day to 7.5 g/day, 5.25 g/day to 7.5 g/day, 5.5 g/day to 7.5 g/day, 5.75 g/day to 7.5 g/day, 6.0 g/day to 7.5 g/day, 6.25 g/day to 7.5 g/day, 6.5 g/day to 7.5 g/day, 6.75 g/day to 7.5 g/day, 7.0 g/day to 7.5 g/day, 7.25 g/day to 7.5 g/day, 0.1 g/day to 5.0 g/day, 0.25 g/day to 5.0 g/day, 0.5 g/day to 5.0 g/day, 0.75 g/day to 5.0 g/day, 1.0 g/day to 5.0 g/day, 1.25 g/day to 5.0 g/day, 1.5 g/day to 5.0 g/day, 1.75 g/day to 5.0 g/day, 2.0 g/day to 5.0 g/day, 2.25 g/day to 5.0 g/day, 2.5 g/day to 5.0 g/day, 2.75 g/day to 5.0 g/day, 3.0 g/day to 5.0 g/day, 3.25 g/day to 5.0 g/day, 3.5 g/day to 5.0 g/day, 3.75 g/day to 5.0 g/day, 4.0 g/day to 5.0 g/day, 4.25 g/day to 5.0 g/day, 4.5 g/day to 5.0 g/day, 4.75 g/day to 5.0 g/day, 0.1 g/day to 2.5 g/day, 0.25 g/day to 2.5 g/day, 0.5 g/day to 2.5 g/day, 0.75 g/day to 2.5 g/day, 1.0 g/day to 2.5 g/day, 1.25 g/day to 2.5 g/day, 1.5 g/day to 2.5 g/day, 1.75 g/day to 2.5 g/day, 2.0 g/day to 2.5 g/day, or 2.25 g/day to 2.5 g/day. In some embodiments, acetaminophen is administered to the subject at a dose of 0.5 g/day to 10 g/day. In some embodiments, acetaminophen is administered to the subject at a dose of 1 g/day to 5 g/day.

In some embodiments, acetaminophen is administered to the subject at a dose of about 0.5 g/day, about 1 g/day, about, about 1.5 g/day, about 2 g/day, about 2.5 g/day, about 3 g/day, about 3.5 g/day, about 4 g/day, about 4.5 g/day, or about 5 g/day. In some embodiments, acetaminophen is administered to the subject at a dose of about 4 g/day.

Combinatorial Use with Other Vaccines

In some embodiments, the present disclosure provides a method of treating or preventing HSV (HSV-1, HSV-2, or a combination thereof) infection

In some embodiments, the present disclosure provides a method of treating or preventing HSV (HSV-1, HSV-2, or a combination thereof) infection comprising administering to a subject in need thereof one or more doses of a therapeutically effective amount of a composition as disclosed herein, wherein the subject is further administered a medically indicated vaccine at least 1 day, at least 2 days, at least 3 days, at least 4 days, at least 5 days, at least 6 days, at least 7 days, at least 8 days, at least 9 days, at least 10 days, at least 12 days, at least 14 days, at least 16 days, at least 17 days, at least 18 days, at least 19 days, or at least 20 days before administration of the therapeutically acceptable amount of the composition. In some embodiments, the subject is further administered a medically indicated vaccine at least 14 days before the administration of the therapeutically acceptable amount of the composition.

In some embodiments, the present disclosure provides a method of treating or preventing HSV (HSV-1, HSV-2, or a combination thereof) infection comprising administering to a subject in need thereof one or more doses of a therapeutically effective amount of a composition as disclosed herein, wherein the subject is further administered a medically indicated vaccine at least 1 day, at least 2 days, at least 3 days, at least 4 days, at least 5 days, at least 6 days, at least 7 days, at least 8 days, at least 9 days, at least 10 days, at least 12 days, at least 14 days, at least 16 days, at least 17 days, at least 18 days, at least 19 days, or at least 20 days after administration of the therapeutically acceptable amount of the composition. In some embodiments, the subject is further administered a medically indicated vaccine at least 14 days after the administration of the therapeutically acceptable amount of the composition.

In some embodiments, a medically indicated vaccine includes, but is not limited to, a vaccine selected from a rabies vaccine, a tetanus vaccine, a hepatitis A vaccine, a hepatitis B vaccine, a measles mumps rubella (MMR) vaccine, a polio vaccine, a diphtheria vaccine, a varicella vaccine, a pertussis vaccine, a shingles vaccine, a pneumococcal vaccine, a human papillomavirus vaccine (HPV), a meningococcal vaccine, and a rotavirus vaccine. In some embodiments, a medically indicated vaccine is a rabies vaccine or a tetanus vaccine.

### XII. Monitoring Efficacy

In some embodiments, the present disclosure provides a method of treating or preventing HSV (HSV-1, HSV-2, or a combination thereof) infection comprising administering to a subject in need thereof one or more doses of a therapeutically effective amount of a composition as disclosed herein, wherein the method further comprises collecting one or more samples from the subject after administration of the one or more doses of a therapeutically effective amount of the composition. In some embodiments, at least one, at least two, at least three, at least four, at least five, at least six, at least seven, at least eight, at least nine, at least ten, at least eleven, at least twelve, at least thirteen, at least fourteen, or at least fifteen samples are collected from the subject after administration of the one or more doses of a therapeutically effective amount of the composition.

In some embodiments, the one or more samples collected from the subject is a blood volume draw.

In some embodiments, a sample is collected from the subject about 120 minutes, about 90 minutes, about 60 minutes, about 45 minutes, about 30 minutes, about 20 minutes, about 15 minutes, about 10 minutes, about 7.5 minutes, about 5 minutes, about 4 minutes, about 3 minutes, about 2 minutes or about 1 minute before administration of a first dose of one or more doses of a therapeutically effective amount of the composition. In some embodiments, a sample is collected from the subject about 1 minute, about 2 minutes, about 3 minutes, about 4 minutes, about 5 minutes, about 7.5 minutes, about 10 minutes, about 15 minutes, about 20 minutes, about 30 minutes, about 45 minutes, about 60 minutes, about 90 minutes, or about 120 minutes after administration of a first dose of one or more doses of a therapeutically effective amount of the composition.

In some embodiments, a sample is collected about 1 week after administration of a first dose of one or more doses of a therapeutically effective amount of the composition. In some embodiments, a sample is collected about 2 weeks after administration of a first dose of one or more doses of a therapeutically effective amount of the composition. In some embodiments, a sample is collected about 4 weeks after administration of a first dose of one or more doses of a therapeutically effective amount of the composition. In some embodiments, a sample is collected about 5 weeks after administration of a first dose of one or more doses of a therapeutically effective amount of the composition. In some embodiments, a sample is collected about 6 weeks after administration of a first dose of one or more doses of a therapeutically effective amount of the composition. In some embodiments, a sample is collected about 8 weeks after administration of a first dose of one or more doses of a therapeutically effective amount of the composition. In some embodiments, a sample is collected about 16 weeks after administration of a first dose of one or more doses of a therapeutically effective amount of the composition. In some embodiments, a sample is collected about 7 months after administration of a first dose of one or more doses of a therapeutically effective amount of the composition. In some embodiments, a sample is collected about 13 months after administration of a first dose of one or more doses of a therapeutically effective amount of the composition.

In some embodiments, a sample is collected from the subject about 120 minutes, about 90 minutes, about 60 minutes, about 45 minutes, about 30 minutes, about 20 minutes, about 15 minutes, about 10 minutes, about 7.5 minutes, about 5 minutes, about 4 minutes, about 3 minutes, about 2 minutes or about 1 minute before administration of a second dose of two or more doses of a therapeutically effective amount of the composition. In some embodiments, a sample is collected from the subject about 1 minute, about 2 minutes, about 3 minutes, about 4 minutes, about 5 minutes, about 7.5 minutes, about 10 minutes, about 15 minutes, about 20 minutes, about 30 minutes, about 45 minutes, about 60 minutes, about 90 minutes, or about 120 minutes after administration of a second dose of two or more doses of a therapeutically effective amount of the composition.

In some embodiments, a sample is collected about 1 week after administration of a second dose of two or more doses of a therapeutically effective amount of the composition. In some embodiments, a sample is collected about 2 weeks after administration of a second dose of two or more doses of a therapeutically effective amount of the composition. In some embodiments, a sample is collected about 1 month after administration of a second dose of two or more doses of a therapeutically effective amount of the composition. In some embodiments, a sample is collected about 3 months after administration of a second dose of two or more doses of a therapeutically effective amount of the composition. In some embodiments, a sample is collected about 6 months after administration of a second dose of two or more doses of a therapeutically effective amount of the composition. In some embodiments, a sample is collected about 12 months after administration of a second dose of two or more doses of a therapeutically effective amount of the composition.

In some embodiments, methods of the present disclosure further comprise a step of measuring levels of HSV (HSV-1 and/or HSV-2) virus-specific neutralizing antibodies in one or more samples collected from the subject. In some embodiments, levels of HSV (HSV-1 and/or HSV-2) virus-specific neutralizing antibodies are measured using any one of a number of assays known to persons of ordinary skill in the art. For example, in some embodiments, levels of HSV (HSV-1 and/or HSV-2) virus-specific neutralizing antibodies are measured using a plaque reduction neutralization test (PRNT). In some embodiments, levels of HSV (HSV-1 and/or HSV-2) virus-specific neutralizing antibodies are measured using a pseudo-viral neutralization test.

In some embodiments, methods of the present disclosure further comprise a step of measuring levels of neutralizing antibodies in one or more samples collected from a subject, where the neutralizing antibodies are specific for one or more HSV (HSV-1 and/or HSV-2) antigens or antigenic fragments thereof encoded by one or more polyribonucleotides in a composition disclosed herein. In some embodiments, levels of neutralizing antibodies are measured using any one of a number of assays known to persons of ordinary skill in the art. For example, in some embodiments, levels of neutralizing antibodies are measured using an enzyme-linked immunosorbent assay (ELISA).

### EXAMPLES

The disclosure is further illustrated by the following examples. The examples are provided for illustrative purposes only. They are not to be construed as limiting the scope or content of the disclosure in any way.

### Example 1: Example LNP Formulations

The present Example describes certain preferred LNP formulations useful for vaccine compositions as described herein.

In some embodiments, LNP formulations that are useful for vaccine compositions as described herein can comprise at least one ionizable aminolipid. In some embodiments, LNP formulations that are useful for vaccine compositions as described herein can further comprise a helper lipid, which in some embodiments may be or comprise a neutral helper lipid. In some embodiments, LNP formulations that are useful for vaccine compositions as described herein can further comprise a polymer-conjugated lipid, for example in some embodiments PEG-conjugated lipids. In some embodiments, LNP formulations that are useful for vaccine compositions as described herein can comprise at least one ionizable aminolipid, at least one helper lipid (e.g., a neutral helper lipid, which in some embodiments may comprise a phospholipid, a steroid, or combinations thereof), and at least one polymer-conjugated lipid (e.g., PEG-conjugated lipid). In some embodiments, an LNP formulation may comprise an ionizable aminolipid, a phospholipid, a steroid, and a PEG-conjugated lipid.

In some embodiments, an ionizable aminolipid may be present in an LNP formulation within a range of 45 to 55 mol percent, 40 to 50 mol percent, 41 to 49 mol percent, 41 to 48 mol percent, 42 to 48 mol percent, 43 to 48 mol percent, 44 to 48 mol percent of total lipids. In some embodiments, an ionizable aminolipid is or comprises ((4-hydroxybutyl)azanediyl)bis(hexane-6,1-diyl)bis(2-hexyldecanoate) (also known as 6-[N-6-(2-hexyldecanoyloxy)hexyl-N-(4-hydroxybutyl)amino]hexyl 2-hexyldecanoate). In some embodiments, an ionizable aminolipid is or comprises SM-102 (heptadecan-9-yl 8 ((2 hydroxyethyl)(6 oxo 6-(undecyloxy)hexyl)amino) octanoate) or an aminolipid as described in Sabnis et al. “A Novel Amino Lipid Series for mRNA Delivery: Improved Endosomal Escape and Sustained Pharmacology and Safety in Non-human Primates” Mol. Ther. (2018) 26:1509-1519. In some embodiments, an ionizable aminolipid is or comprises an ionizable aminolipid as disclosed in US2020/0163878 or WO2018/078053, the entire contents of each of which are incorporated herein by reference for the purposes described herein.

In some embodiments, a phospholipid may be present in an LNP formulation within a range of 5 to 15 mol percent, 7 to 13 mol percent, or 9 to 11 mol percent of total lipids. In some embodiments, a phospholipid is or comprises 1,2-Distearoyl-sn-glycero-3-phosphocholine (DSPC).

In some embodiments, a sterol may be present in an LNP formulation within a range of 30 to 50 mol percent, 35 to 45 mol percent or 38 to 43 mol percent of total lipids. In some embodiments, a sterol is or comprises cholesterol.

In some embodiments, a polymer conjugated lipid (e.g., PEG-conjugated lipid) may be present in an LNP formulation within a range of 1 to 10 mol percent, 1 to 5 mol percent, or 1 to 2.5 mol percent of total lipids. In some embodiments, a PEG-conjugated lipid is or comprises 2-[(polyethylene glycol)-2000]-N,N-ditetradecylacetamide (also known as 2-[2-(@-methoxy (polyethyleneglycol2000) ethoxy]-N,N-ditetradecylacetamide). In some embodiments, a phospholipid is or comprises PEG2000-DMG (1-monomethoxypolyethyleneglycol-2,3-dimyristylglycerol with polyethylene glycol of average molecular weight 2000). In some embodiments, a PEG-conjugated lipid is or comprises a PEG-lipid as disclosed in US2020/0163878 or WO2018/078053, the entire contents of each of which are incorporated herein by reference for the purposes described herein.

In some embodiments, a LNP formulation comprises (i) an ionizable aminolipid within a range of 45 to 55 mol percent of total lipids; (ii) a phospholipid within a range of 8 to 12 mol percent of total lipids; (iii) a steroid within a range of 35 to 45 mol percent of total lipids; and (iv) a polymer conjugated (e.g., PEG-conjugated polymer) within a range of 1 to 2 mol percent of total lipids; and RNA molecules as described herein that are encapsulated within or associated with the lipid nanoparticles.

In some embodiments, a LNP formulation comprises (i) ionizable amino lipid within a range of 45 to 55 mol percent of total lipids; (ii) DSPC within a range of 5 to 15 mol percent of total lipids; (iii) cholesterol within a range of 35 to 45 mol percent of total lipids; and (iv) a PEG-conjugated lipid within a range of 1 to 2 mol percent of total lipids; and RNA molecules as described herein that are encapsulated within or associated with the lipid nanoparticles.

In some embodiments, a LNP formulation comprises (i) an ionizable aminolipid within a range of 40 to 50 mol percent of total lipids; (ii) a phospholipid within a range of 5 to 15 mol percent of total lipids; (iii) a steroid within a range of 35 to 45 mol percent of total lipids; and (iv) a polymer conjugated (e.g., PEG-conjugated polymer) within a range of 1 to 10 mol percent of total lipids; and RNA molecules as described herein that are encapsulated within or associated with the lipid nanoparticles. In some such embodiments, an ionizable aminolipid is or comprises ((4-hydroxybutyl)azanediyl)bis(hexane-6,1-diyl)bis(2-hexyldecanoate) (also known as 6-[N-6-(2-hexyldecanoyloxy)hexyl-N-(4-hydroxybutyl)amino]hexyl 2-hexyldecanoate). In some such embodiments, a phospholipid is or comprises 1,2-Distearoyl-sn-glycero-3-phosphocholine (DSPC). In some such embodiments, a steroid is or comprises cholesterol. In some such embodiments, a polymer conjugated polymer is or comprises 2-[(polyethylene glycol)-2000]-N,N-ditetradecylacetamide (also known as 2-[2-(@-methoxy (polyethyleneglycol2000) ethoxy]-N,N-ditetradecylacetamide).

In one embodiment, a LNP formulation comprises the following lipids included in Table 17 below and RNA molecules as described herein.

TABLE 17
Example LNP Formulation

| | Proportion |
|---|---|
| Lipids | (mol %) |
| ((4-hydroxybutyl)azanediyl)bis(hexane-6,1-diyl)bis(2- | 47-49 (e.g., 47.5) |
| hexyldecanoate) (also known as 6-[N-6-(2- | |
| hexyldecanoyloxy)hexyl-N-(4-hydroxybutyl)amino]hexyl 2- | |
| hexyldecanoate) | |
| 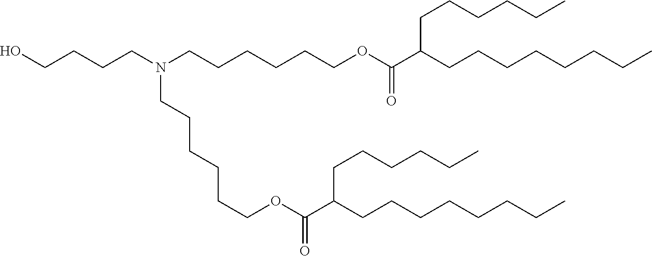 [SMILES: CCCCCCCCC(CCCCCC)C(=O)OCCCCCCN(CCCCO)CCCCCCOC(=O)C(CCCCCC)CCCCCCCC] | |
| 2-[(polyethylene glycol)-2000]-N,N-ditetradecylacetamide | 1.5-2.0 (e.g., 1.8) |
| (also known as 2-[2-(ω-methoxy (polyethyleneglycol2000) | |
| ethoxy]-N,N-ditetradecylacetamide) | |
| 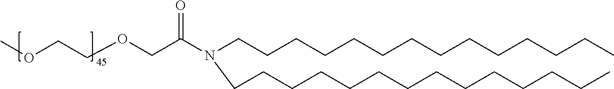 | |
| DSPC | 9.5-10.5 (e.g., 10) |
| Cholesterol, synthetic | 39-42 (e.g., 40.7) |

In some embodiments, an LNP formulation comprises an ionizable aminolipid, DSPC, cholesterol, and PEG-conjugated lipid at a molar ratio of approximately 50:10:38.5:1.5 or 47.5:10:40.8:1.7. In some embodiments, an ionizable amino lipid is or comprises ((4-hydroxybutyl)azanediyl)bis(hexane-6,1-diyl)bis(2-hexyldecanoate) (also known as 6-[N-6-(2-hexyldecanoyloxy)hexyl-N-(4-hydroxybutyl)amino]hexyl 2-hexyldecanoate).

In some embodiments, an LNP formulation comprises (i) SM-102 (heptadecan-9-yl 8 ((2 hydroxyethyl)(6 oxo 6-(undecyloxy)hexyl)amino) octanoate) within a range of 45 to 55 mol percent of total lipids; (ii) DSPC within a range of 5 to 15 mol percent of total lipids; (iii) cholesterol within a range of 35 to 45 mol percent of total lipids; and (iv) PEG2000-DMG within a range of 1 to 2 mol percent of total lipids; and RNA molecules as described herein that are encapsulated within or associated with the lipid nanoparticles.

In some embodiments, an LNP formulation comprises (i) ((4-hydroxybutyl)azanediyl)bis(hexane-6,1-diyl)bis(2-hexyldecanoate) (also known as 6-[N-6-(2-hexyldecanoyloxy)hexyl-N-(4-hydroxybutyl)amino]hexyl 2-hexyldecanoate) within a range of 45 to 55 mol percent of total lipids; (ii) DSPC within a range of 5 to 15 mol percent of total lipids; (iii) cholesterol within a range of 35 to 45 mol percent of total lipids; and (iv) a PEG-conjugated lipid within a range of 1 to 2 mol percent of total lipids; and RNA molecules as described herein that are encapsulated within or associated with the lipid nanoparticles.

### Example 2: Example Characterization Studies

The present Example describes certain potential characterization studies that may be utilized, for example, to identify, select, and/or characterize vaccine candidates or vaccine compositions (e.g., manufacturing batches thereof), or components thereof as described herein.

A potential immunization protocol that can be utilized to assess ability of a vaccine candidate, such as a trivalent vaccine, that comprises or delivers an HSV-2 antigen(s) as described herein to induce B- and/or T-cells, e.g., after intramuscular immunization, directed to the antigen(s) and/or epitope(s) thereof. In some embodiments, level and/or diversity of response is determined. In some embodiments, presence and/or level of neutralizing antibodies is/are determined. In some embodiments, protection of the immunized subject from challenge with HSV is assessed.

Alternatively or additionally, in some embodiments, one or more in vitro assessments may be performed, for example:
(1) in vitro expression of an antigen encoded by an RNA included in a vaccine composition; and/or
(2) in vitro potency of antigen expressed from an RNA included in a vaccine composition as described herein.

### Example 3: Example Pre-Clinical Assessment

The present Example describes certain pre-clinical assessments that may be performed of certain Trivalent Vaccine described herein:

In some embodiments, one Trivalent Vaccine candidate is assessed. In some embodiments, more than one different Trivalent Vaccine candidate may be assessed. In some such embodiments, different candidates may vary, for example, in:
RNA platform (e.g., unmodified RNA, modified RNA, saRNA);
Encoded antigen(s);
Number of RNAs;
Elements of RNA construct (e.g., cap and/or cap-adjacent sequences, 5′-UTR, 3′-UTR, and/or PolyA tail); and/or
Lipid composition of LNP.

In some embodiments, pre-clinical assessment of certain RNA vaccine compositions (e.g., LNP formulated RNA-based HSV vaccines) comprises one or more of assessment in challenge experiments, assessment of level of protection, assessment of immunogenicity, and/or assessment of functional antibody responses.

LNP formulated RNA-based HSV vaccines are tested in a challenge model. Non-human primate models, such as Rhesus macaques and Cynomolgus monkey, and/or rodent models, such as C57/B16 mice, Balb/c mice or NODscidIL2Rγnull mice; and/or guinea pig models, inoculated with HSV, are administered a first vaccination and can be administered an additional vaccination (e.g., 1, 2, 3, 4, 5, 6, 7, 8, 9, or 10 additional vaccinations) following the first vaccination. Wherein more than one vaccination is administered, the vaccinations are administered at an interval of 1, 2, 3, 4, 5, 6, 7, or 8 week intervals). Following vaccination, animals are challenged by HSV. Alternatively or additionally, animals are challenged by intravenous, subcutaneous, and/or intramuscular injection of virus-infected lymphocytes. Lymphocytes can be infected with any suitable strain of HSV. Animals are then evaluated for reduced infection of neurons. In some embodiments, an animal model is challenged in a plurality of instances (e.g., before first vaccination and/or wherein additional vaccinations are administered, at any time point between or after vaccinations). Following challenge, animals subjected to the study may be assessed according to any method known in the art, including, for example, serology assessment, immunogenicity, level of protection, etc.

In some embodiments, serum antibody characterization and/or serum transfer experiments (e.g., from one vaccinated species to a different non-vaccinated species, e.g., from vaccinated non-human primate to non-vaccinated mouse) are conducted (e.g., to assess protective antibody response).

In some embodiments, certain RNA vaccine compositions of the present disclosure are assessed for level of protection. Level of protection can be assessed according to any suitable method known in the art.

In some embodiments, certain RNA vaccine compositions of the present disclosure are assessed for immunogenicity. For example, ELISA can be used to determine IgG specific (and subclasses thereof) titers and/or avidity of antibodies generated in response to certain RNA vaccine compositions of the present disclosure to HSV antigens. In some embodiments, serum antibody titers against HSV glycoprotein (e.g., gC, gD, and/or gE glycoprotein, etc.) is determined by ELISA using standard methods. In some embodiments, for example, ELISpot (e.g., for CD8+, CD4+ T cells and/or IFNγ) and assessment of pro-inflammatory cytokine responses with splenocytes from immunized and/or challenged animal models and peptide pools derived from vaccine targets can also be assessed. In some embodiments, for example, phenotyping of immune responses (e.g., by flow cytometry) are assessed. In some embodiments, for example, T cell depletion and/or protection assays are conducted to assess immunogenicity (e.g., according to any suitable known method in the art).

In some embodiments, one or more functional responses of antibodies generated in response to certain RNA vaccine compositions of the present disclosure are assessed. Functional antibody responses can be assessed, for example, using an HSV neutralization assay. In some embodiments, an HSV in vitro neutralization assay is performed to evaluate one or more anti-HSV glycoprotein (e.g., HSV gC, gD gE, or a combination hereof) antibodies in neutralizing HSV. For example, anti-HSV glycoprotein antibodies are obtained by collecting the sera of animals (e.g., mice) vaccinated with HSV RNA vaccines. HSV virus are added to the diluted sera and neutralization is allowed to continue for 1 hour at room temperature. 3T3 cells are seeded in 96-wells one day before and the virus/serum mixtures are added to 3T3 monolayers. The cells are fixed on the next day and HSV-specific staining is performed. The plates are scanned and analyzed. A neutralization titer is expressed as the highest serum dilution required to achieve a 50% reduction in the number of plaques.

In some embodiments, functional antibody responses can be assessed, for example, using passive transfer studies of sera from immunized animals to naïve animals that are challenged and assessing level of protection.

### Example 4: In Vitro testing

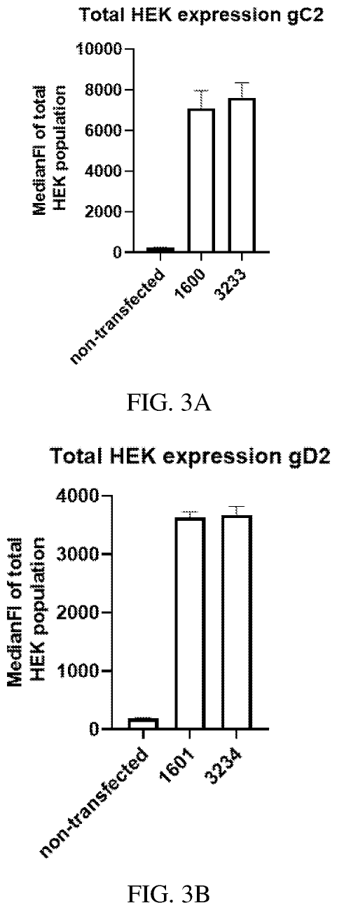

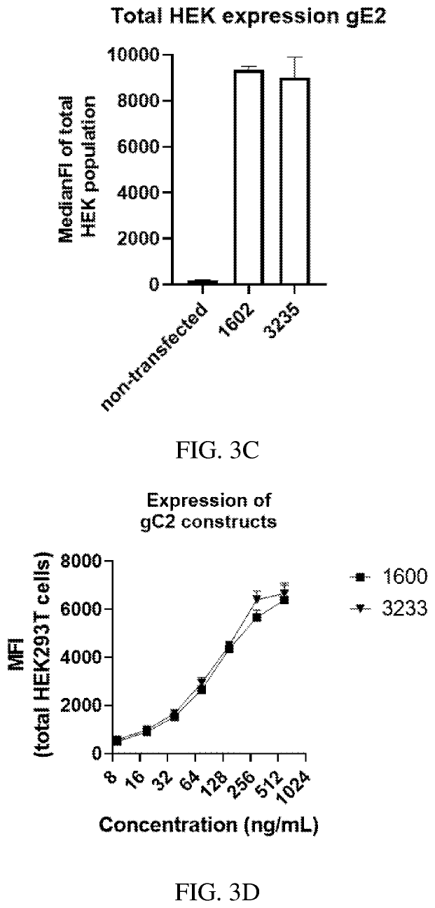

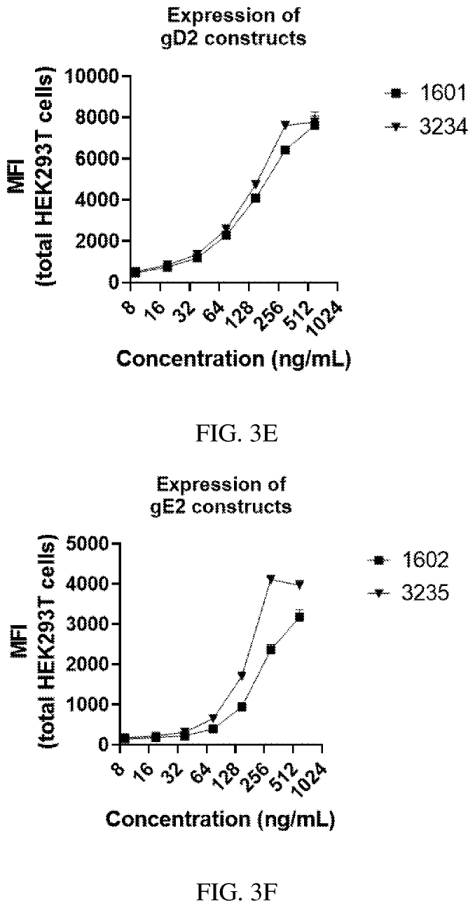

The present Example provides in vitro transfection and expression data in HEK293T cells transfected with nucleoside-modified RNA (modRNA) encoding HSV-2 gC (gC2), gD (gD2), and/or gE (gE2) antigens. In the present Example, HEK293T cells were transfected with:
0.2 μg/mL modRNA (SEQ ID NO: 106 or 349) encoding a gC2 construct (SEQ ID NO: 65 or 159, respectively) using a commercial transfection reagent (FIG. 3A);
0.2 μg/mL modRNA (SEQ ID NO: 118 or 352) encoding a gD2 construct (SEQ ID NO: 70) using a commercial transfection reagent (FIG. 3B);
0.4 μg/mL modRNA (SEQ ID NO: 123 or 353) encoding a gE2 construct (SEQ ID NO: 73 or 75, respectively) using a commercial transfection reagent (FIG. 3C); or
0.2 μg/mL modRNA (SEQ ID NO: 106 or 349) encoding a gC2 construct (SEQ ID NO: 65 or 159, respectively), 0.2 μg/mL modRNA (SEQ ID NO: 118 or 352) encoding a gD2 construct (SEQ ID NO: 70) and 0.4 μg/mL modRNA (SEQ ID NO: 123 or 353) encoding a gE2 construct (SEQ ID NO: 73 or 75, respectively) where the modRNAs are in a LNP formulation (FIGS. 3D-F).

After transfection, cells were incubated to express the modRNA for 18 h. Afterwards, cells were harvested and probed with antigen-specific mouse monoclonal antibodies and a secondary fluorescence-tagged anti-mouse antibody to detect expression of vaccine antigens in HEK293T cells. The median fluorescence intensity (MFI) measured in FACS serves as a surrogate for antigen expression.

Briefly, 0.4×10⁶ HEK293T cells were seeded 6 hours prior to transfection in 12-well plates. modRNAs encoding for HSV-2 gC, HSV-2 gD, or HSV-2 gE were formulated using Lipofectamine™ MessengerMAX™ (ThermoFisher Scientific) according to the manufacturers' instructions prior to transfection.Trivalent modRNA encoding for HSV-2 gC, HSV-2 gD, and HSV-2 gE was LNP-formulated. Cells were incubated for 18 hours at 37° C. and 5% CO₂ prior to staining. Afterwards, cells were harvested and incubated with a viability dye (eBioscience™ Fixable Viability Dye eFluor™ 450, ThermoFisher Scientific), fixed (Fixation Buffer, BioLegend), permeabilized (Permeabilization Buffer, eBioscience) and incubated with a mouse anti-gC, gD or gE antibody and subsequently stained with an Alexa Fluor® 647 AffiniPure Donkey Anti-Mouse IgG (H+L) secondary antibody (Jackson ImmunoResearch). Cells were acquired using a BD FACSCelesta 2 (BD) and data was analyzed with FlowJo V10.8 (BD).

As demonstrated in FIG. 3, all vaccine antigens were delivered to and expressed in HEK293T cells (FIGS. 3A-F).

High transfection frequencies and strong expression was observed with gC2, gD2 and gE2 single transfections using a commercial transfection reagent and transfection using LNP formulated trivalent modRNA. Both transfection methods also showed comparable transfection frequencies and expression levels when comparing the three different antigens. These results demonstrate that (i) modRNAs described herein can induce strong antigen expression (FIGS. 3A-F), and (ii) modRNAs described herein can be administered in combination without significantly interfering with transfection efficiency or antigen expression (FIG. 3D-F).

### Example 5: Confirmatory Pharmacology Study of Trivalent Vaccine in Guinea Pigs

The objective of the study summarized in the present Example was to analyze the tolerability, immunogenicity, and disease protection of a combination comprising a polyribonucleotide encoding an HSV-2 glycoprotein C (gC), a polyribonucleotide encoding an HSV-2 glycoprotein D (gD) and a polyribonucleotide encoding an HSV-2 glycoprotein E (gE) and formulated in lipid nanoparticles comprising ALC-0315 (4-hydroxybutyl) azandiyl bis(hexane-6,1-diyl)bis(2-hexyl decanoate). In the present Example, the composition tested comprised three polyribonucleotides: a first comprising SEQ ID NO:106 (construct 1600) and encoding a polypeptide having an amino acid sequence according to SEQ ID NO: 65, a second comprising SEQ ID NO: 118 (construct 1601) and encoding a polypeptide having an amino acid sequence according to SEQ ID NO: 70, and a third comprising SEQ ID NO: 123 (construct 1602) and encoding a polypeptide having an amino acid sequence according to SEQ ID NO: 73.

In guinea pigs, the composition induced potent antigen-specific immunoglobulin G (IgG) antibodies in serum and vaginal mucosa, as well as high titers of neutralizing antibodies in serum. 100% protection was provided against a highly lethal intravaginal HSV-2 challenge, while 70% of animals succumbed to viral challenge in the control group. Furthermore, days and severity of genital lesions as well as urinary retention were reduced in immunized animals and vaginal titers tend to be decreased on day 2 and 4 after challenge compared to the unvaccinated control group. The results of the study support the use of the tested HSV-2 vaccine as a clinical candidate for human trials.

### BACKGROUND

Genital herpes, caused by HSV, is a sexually transmitted infection. To date, a vaccine has not been licensed to prevent genital lesions resulting from herpes simplex virus-1 (HSV-1) or 2 (HSV-2) infection, despite extensive efforts by major pharmaceutical companies. The strategy behind the RNA vaccine candidate tested in the present Example is to induce immune responses that block HSV-2 cellular entry, inhibit cell-to-cell spread, and counteract HSV-2's inhibitory effects on the complement system and virus clearance. The vaccine candidate characterized herein includes three HSV-2 immunogens, namely HSV-2 glycoproteins C, D, and E (HSV-2 gC, HSV-2 gD, and HSV-2 gE), formulated with ALC-0315 lipid nanoparticles.

An earlier generation of trivalent HSV-2 gC, HSV-2 gD, and HSV-2 gE vaccine containing recombinantly expressed ectodomains of these glycoproteins in a baculovirus system, formulated with CpG oligonucleotides and an aluminum salt as adjuvant, demonstrated immunogenicity and efficacy in mice. However, guinea pigs immunized with the same trivalent protein vaccine still developed subclinical infection based on vaginal shedding of HSV-2 DNA during the recurrent phase of infection (Awasthi et al. 2017, Hum Vaccin Immunother. 2017 Dec. 2; 13 (12): 2785-2793). To improve vaccine efficacy, the same three glycoprotein antigens were produced by immunizing mice and guinea pigs with an RNA-LNP vaccine instead of recombinant protein. Superior immunogenicity and efficacy of the RNA-LNP vaccine compared to the baculovirus glycoprotein CpG/alum vaccine was shown in both mice and guinea pigs (Awasthi et al. 2019, Sci Immunol. 2019 Sep. 20).

Previous studies found that formulation of the RNA vaccine with ALC-0315 lipid nanoparticles (RNA-LNP315) increased immunogenicity compared to formulation with ALC-0307 (RNA-LNP307). Therefore, ALC-0315 was chosen as a component of the LNP formulation used in the present example.

Additionally, as previous experiments showed complete protection in most guinea pigs, lower doses, and a prime boost regimen (without a second boost) were chosen in this study. This approach allows coverage of a broader range of disease severity when guinea pigs were challenged with a highly lethal dose of HSV-2 virus.

### Objective

The objective of this study was to confirm tolerability, immunogenicity, and disease protection of an HSV-2 RNA vaccine candidate (a combination comprising a polyribonucleotide encoding an HSV-2 glycoprotein C (gC), a polyribonucleotide encoding an HSV-2 glycoprotein D (gD) and a polyribonucleotide encoding an HSV-2 glycoprotein E (gE)) in guinea pigs.

### Study Design

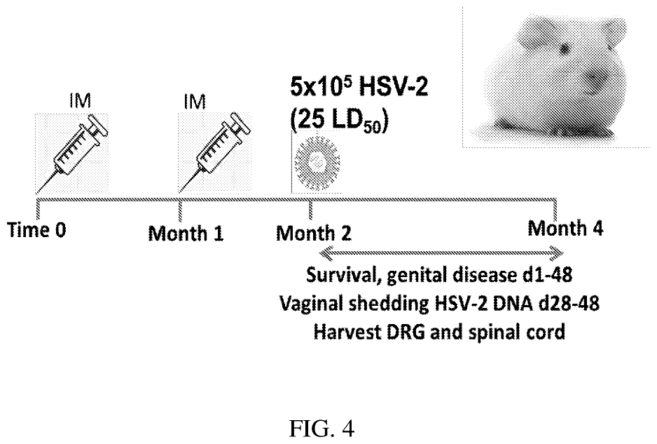

Thirty female Hartley guinea pigs, weighing 300 to 350 g, were divided into three experimental groups (10 animals each) and immunized on day 0 and day 28 (PBS as control, and 3 μg or 15 μg of RNA vaccine, see Table 18 below). The study design is displayed in FIG. 4.

TABLE 18
Experimental groups and treatment

| Group | Test article | Dose | Immunization | Challenge |
|---|---|---|---|---|
| 1 | PBS | — | | Approx. |
| 2 | Trivalent | 3 μg | | 1 month after last |
| | Vaccine | | | immunization / |
| 3 | Trivalent | 15 μg | IM | 5 × 10⁵ PFU of |
| | Vaccine | | day 0 and | HSV-2 strain |
| | | | day 28 | MS (25-fold LD₅₀) |

HSV-2 MS = herpes simplex virus-2 strain;
MS;
IM = intramuscular;
LD = lethal dose;
PFU = plaque forming units

Blood samples were collected at day 56, 4 weeks after the second immunization, for analysis of HSV-2 specific antibody responses. Levels of IgG antibodies against HSV-2 gC, HSV-2 gD, and HSV-2 gE were measured by ELISA. Neutralizing antibody titers were determined using a serum HSV-2 plaque reduction assay and defined as highest dilution of serum with 5% human complement that reduced the number of HSV-2 plaques by 50%. Vaginal swabs were also collected 4 weeks after the second immunization for analysis of mucosal antibody titers. Vaginal swabs were placed into 100 μL PBS and a serial two-fold dilution was tested for HSV-2 gC, HSV-2 gD and HSV-2 gE IgG by ELISA.

At day 60, animals were challenged with a lethal dose of 5×10⁵ plaque forming units (PFU) of HSV-2 strain MS (25-fold LD₅₀) and scored for survival and signs of genital disease for a period of 48 days. Body weights were recorded daily for the first 2 weeks post-infection. Day 2 and day 4 vaginal titers, and vaginal shedding of HSV-2 DNA between days 28 to 48 post-infection (p.i.) were also determined. Vaginal shedding of HSV-2 DNA was analyzed by a qPCR assay, together with HSV-2 DNA copy in DRG (dorsal root ganglia) and spinal cord at day 48.

### Materials and Methods

Test items included three codon-optimized, 5′capped, modRNA constructs encoding for HSV-2 glycoprotein C (gC), D (gD) and E (gE), encapsulated at a 1:1:1 ratio within ALC-0315 (LNP315). Sterile PBS was used as control.

### Animal Care

Guinea pigs were pair housed under a 12:12-h light: dark cycle in 2000P Tecniplast cages (Buguggiate, Italy), on cellulose fiber animal bedding in an ABSL-2 vivarium. The room temperature was maintained between 68 to 79° F. and humidity 30 to 70% with air exchange 10 to 15/h in the room. Guinea Pig Diet 5025 (LabDiet, Richmond, IN), Oxbow timothy hay, and reverse osmosis water were provided ad libitum and cages were changed twice weekly. All husbandry materials were autoclaved prior to use.

Routine animal monitoring was carried out daily and included inspection for dead animals and control of food and water supplies. Each animal's health was monitored at least twice weekly prior to HSV-2 infection and daily after HSV-2 infection until the end of the experiment. The general physical condition was assessed with the following parameters:
Body weight loss ≥20%
Labored/gasping breathing
Signs of lethargy
Pain when handled
Genital disease
Hindlimb weakness
Urinary retention

### Treatment Schedule, Route of Administration, and Dose

Test items were diluted in PBS and administered via IM injection on day 0 and day 28 (see Table 18 above). The control group was injected with sterile PBS. 50 μL was injected into the right hind muscle using an insulin syringe (29-gauge needle).

Blood was sampled via the lateral saphenous vein in the left hind limb. In short, the animal was held face down with the left lateral aspect of the hind limb facing the person performing the bleed. The fur off the left hind leg was shaved up to the knee and Vaseline was applied. A 20-gauge needle was used to prick the vein and blood droplets (totaling between 100 to 1000 μL) were collected into an Eppendorf tube. To allow the blood to clot, the samples were placed on ice for at least 30 min. Blood samples were centrifuged at 8000 RPM for 10 min. The supernatant (serum) was collected and stored at −20° C.

Immunized guinea pigs were challenged with a dose of 5×10⁵ PFU HSV-2 MS at day 60, approximately 1 month after the last immunization. On the day of infections, viral stocks of HSV-2 (strain MS from the American Type Culture Collection [ATCC]) were thawed on ice. Thawed stocks were then diluted to the desired concentration (5×10⁵ PFU/50 μL) with sterile Dulbecco's Modified Eagle's medium (DMEM) tissue culture media. A polyester swab (Puritan) dipped into phosphate buffered saline (PBS) was used to clear the mucus from the vaginal vault. The guinea pig was held with the head slightly down and 25 μL of HSV-2 virus was inoculated into the vaginal vault using a soft catheter attached to a pipette tip. The guinea pig was held in position for a further 30 to 60 second and then placed back into the cage. A second HSV-2 inoculation was performed 1 hour later to inoculate the remaining 25 μL.

Guinea pigs were scored for signs of acute disease on days 1 to 14 after infection and for signs of recurrent disease on days 15 to 48. Naive non-immunized guinea pigs are expected to show clear indications of genital herpes disease 3 to 4 days after infection.

For scoring the severity of genital disease, a score of 1 to 4 was assigned:
Score 1:1 to 2 distinct genital lesions
Score 2: >2 distinct genital lesions
Score 3: coalesced lesions
Score 4: ulcerated lesions
Animals are evaluated daily for urinary retention (recorded when present)
Animals are observed daily for evidence of hindlimb weakness (recorded when present)

On day 2 and 4 post-infection, vaginal swabs were performed to document infection in the vaginal canal. Replicating virus was titrated using plaque assay. The cotton swab was inserted into the vaginal cavity and rotated six times to collect the tissue. The swab was then removed and placed in 1 ml of swab media (DMEM with 5% heat-inactivated FBS, 1% L-Glut, 1% Antibiotic/antimycotic, 0.4% Gentamycin, 0.025% Vancomycin and 1.5% HEPES). The aluminum handle was clipped, and the swab was left in the tube. The tubes were stored at −80° C.

### Vaginal Wash Procedure to Obtain IgG Titers in ELISA

Vaginal secretions for neutralizing titers were obtained using an eye spear swab (BVI). The eye spear was trimmed 1 mm from each side and inserted into the guinea pig vaginal cavity for 45-60 s. A small hole was pricked in the bottom of a 0.5 ml reaction tube using a 20-gauge needle. Next, the spear was placed into this tube, and both together were placed into a 1.5 ml reaction tube. 100 μl of PBS were added directly onto the spear and the tubes were centrifuged for 3 min at 8000 RPM. The vaginal fluid and PBS were collected in the 1.5 mL tube and stored at −80° C.

### Isolation of Dorsal Root Ganglia (DRG) and Spinal Cord

Spinal cord and DRG were isolated at the time of termination of experiment. Guinea pigs were anesthetized with Euthasol (IP) at 150 mg/kg of body weight. Once they lost consciousness (confirmed by toe pinch), cardiac bleed (~10 ml) was performed. The animal's back was shaved, and the skin lifted to expose the spinal column. The spinal column was cut out along with muscle on either side from mid-back to the tail. Next, the spinal column was opened longitudinally with sharp surgical scissors to expose the spinal cord. A portion of the spinal cord (about ⅝″ from the lower end) was collected into a 1.5 ml tube containing 1 ml DMEM media and stored at −80° C.

Next, the remaining spinal cord was gently removed to expose the DRG on either side of vertebral column. The entire DRG root (not just the DRG) was pulled from the vertebral canals of lumbosacral region by going deep into the vertebral canals. Each ganglion was placed in 1 ml DMEM media in a 1.5 ml tube. Tubes were stored at −80° C. until further use.

### Endpoint of Experiment/Termination Criteria

Animals were euthanized in accordance with the Panel on Euthanasia of the American Veterinary Medical Association by intraperitoneal injection of Euthasol euthanasia solution (1 mL for up to a 1 kg guinea pig). Additionally, termination criteria applied according to the recommendation of Panel on Euthanasia of the American Veterinary Medical Association. Body weight losses exceeding 20%, or a high severity level in any of the other categories described below were on their own sufficient reason for immediate euthanasia.

### ELISA Protocol to Detect IgG Antibody Responses to HSV-2 gC, HSV-2 gD, and HSV-2 gE in Guinea Pig Sera and Vaginal Wash Samples

Serum and vaginal wash samples were tested in 96-well plates to detect HSV-2 gC-, HSV-2 gD-, and HSV-2 gE-specific antibody concentrations. Briefly, 4 weeks after the last injection, serum and vaginal wash samples were analyzed by endpoint titration. HSV antigens (recombinant HSV-2 gC, HSV-2 gD, and HSV-2 gE from a Baculovirus-expression system) were diluted to a final concentration of 1 μg/mL in 50 mM sodium bicarbonate binding buffer, pH 8.5-9.0. Wells of a 96-well ELISA plate were coated with 100 μl of HSV antigen (100 ng/well). Thereafter, the plate was covered with adhesive plastic and incubated overnight at 4° C. After aspirating the coating solution, the plate was washed three times using the Wellwash with 300 μL PBS/0.05% Tween-20 (PBST). Remaining drops were removed by blotting the plate on a paper towel. Wells were blocked by adding 250 μL of 5% milk in PBST per well. The plate was covered with adhesive plastic and incubated for 2 hours at room temperature with gentle agitation.

During the blocking step, guinea pig sera was diluted 1:500 in PBST. Thereafter, two-fold serial dilutions were performed in PBST so that the following dilutions were available to add to the plate: 1:500, 1:1,000, 1:2,000, 1:4,000, 1:8,000, 1:16,000, 1:32,000, 1:64,000, 1:128,000, 1:256,000, 1:512,000, and 1:1,024,000. Vaginal wash was diluted 1:50 in PBS. Once blocking was complete, the plate was washed three times with 300 μL PBST and blotted dry on a paper towel. Then, 100 μL of the diluted sera or vaginal wash was added to each well. The plate was covered with an adhesive sheet and incubated for 1 hour at room temperature with gentle agitation. Secondary antibody (rabbit anti-guinea pig IgG) was prepared by diluting 1:2,000 in PBST. The plate was washed three times with 300 μL PBST, blotted dry on a paper towel, and 100 μL of the secondary antibody was added to each well of the ELISA plate. After covering the plate with an adhesive sheet, it was incubated for 30 min at room temperature with gentle agitation. The plate was washed three times with 300 μL PBST, blotted dry on a paper towel, and 100 μL of ABTS (1-Component Microwell Peroxidase Substrate) was added to each well of the ELISA plate. Again, the plate was covered with foil and incubated for 30 min at room temperature with gentle agitation. Subsequently, 100 μL of stop solution (1×) were added per well.

The absorbance was measured within 30 minutes (MRX Revelation) at 405 to 410 nm (405 nm). Endpoint titers were calculated as the serum dilution giving an optical density (OD) reading >0.1 and at least 2-fold higher than background OD. Background OD-wells were treated with everything but using PBS instead of guinea pig sera or vaginal wash.

### Neutralization Antibody Titer Using Complement by Plaque Reduction Assay

The aim of this analysis was to determine the dilution of serum from vaccinated and mock vaccinated guinea pigs required for 50% neutralization of the HSV-2 virus in the presence of human complement. The source of complement was whole serum from an HSV-1/HSV-2 negative human individual.

For this assay, Vero cells were seeded on 24-well plates at a dilution of 2×10⁵ cells per well overnight. The following day, 2-fold serum dilutions ranging from 1:10 to 1:10,240 were prepared in DMEM in a volume of 50 μL and mixed with 45 μL of virus solution containing 100 PFU and 5 μL of human complement. For the initial 1:10 serum dilution, 10 μL of undiluted sera was mixed with 40 μL of media, 45 μL of virus and 5 μL of human complement. The sera, virus and complement were mixed in a 96-well u-bottom plate by gentle rocking at 37° C. for 1 hour. As a negative control for neutralization, PBS was mixed with HSV-2 and human complement.

Then, the media from the Vero cells was replaced with 100 μL of the mixture containing serum (or PBS), virus, and complement, and incubated at 37° C. for 1 hour with rocking every 10 minutes to ensure that the cells did not dry out.

After 1 hour, the 100 μL virus/serum mix was aspirated and replaced with 1 mL of 1.5% CMC-DMEM per well. The plate was then incubated at 37° C. for 68 to 72 hours.

For staining of the plaques, the CMC-DMEM overlay was gently aspirated and 0.5 mL/well crystal violet dye (0.05% w/v) was added. The plate was incubated at room temperature for 30 min to 1 h. The crystal violet dye was aspirated and 1 mL water/well was added to remove traces of the dye. Thereafter, the water was aspirated, and the remaining water was removed by inverting plate on a blotting paper and allowing the plate to air-dry. Plaques were counted under the inverted light microscope.

Dilution of serum required to reduce virus plaques by 50% was considered as the neutralization titer for that serum sample.

Virus Titers of Vaginal Swabs from Guinea Pig by Plaque Assay

This protocol assumed that at least 1×75 cm² flask of Vero cells ready for splitting were available before the plaque assay. Briefly, swab sample dilutions were prepared in a 96-well plate such that one 96-well plate corresponded to four 24-well plates (i.e., A1-A6=plate 1, A7-A12=plate 2, E1-E6=plate 3, and E7-E12=plate 4). 225 μL of swab medium was aliquoted to each 24-well plate. Undiluted stock swab virus was prepared by adding 250 μL of the thawed swab samples to 225 μL of swab medium in the corresponding well. The −1 dilution was prepared by adding 25 μL of stock swab virus to 225 μL of swab medium. This procedure was repeated to obtain the −2, and −3 dilutions of the swab virus. Vero cells in a 24-well plate (~80% confluent) were infected with the swab virus samples starting with the −3 dilution through undiluted stock swab virus. The plate was rocked 10 × and placed in an incubator. Rocking was repeated every 5 min for the first 15 min followed by every 15 min for a total of 1 h. Next, 0.5 mL of CMC-DMEM overlaying medium pre-warmed to 37° C. was added to each well. Plates were incubated at 37° C. for 72 hours.

Following this, the incubating cells were fixed and stained by adding enough 0.05% crystal violet such that the bottom of the plate was covered and allowed to stain for 3 hours. The next day plaques were counted by scoring the plate in a grid-pattern like using a hemocytometer (i.e., cells touching top and left grid lines) under a microscope. The dilution at which 10-30 plaques per well were counted was the dilution factor (e.g., dilution factor=10⁻² if 10 to 30 plaques were counted in the −2 dilution). Swab titer was calculated by taking the plaque count and multiplying by the 1×10ⁿ to get PFU/200 μL where n is the dilution factor e.g., 14 plaques at 10⁻² dilution is equal to 14×10⁻²=1400 PFU/200 μL. Finally, swab titer was reported PFU/mL by multiplying PFU/200 μL by five.

qPCR on DNA in Vaginal Swabs and Isolated DRG and SP

qPCR assays on guinea pig vaginal swab samples, as well as in samples from isolated DRG and SP were performed in duplicate for HSV-2 DNA. Separate reactions were used to amplify HSV-2 DNA and the guinea pig GAPDH. The DRG HSV-2 DNA copy number was expressed as log 10 DNA copies per 106 guinea pig GAPDH genes. The HSV-2 qPCR assay was designed to detect low levels of HSV-2 DNA in swab samples from guinea pig in vivo studies. The primers and probes used in this assay were designed from published sequences to type-specific and type-common genes of HSV-2. Specific primers and probes were also designed from published sequences to host housekeeping genes to monitor consistency of sampling and to allow for normalizing of final results. (See Table 19 below, for primer and probe sequences).

The quantification method used a standard curve that was prepared from a dilution series of control template of known concentration. The standard curve was generated by plotting the logo of the initial template copy number against the cycle threshold (Ct) value generated at each dilution. Ct values from samples were then compared to the template controls of the standard curve and a template quantity was then estimated.

TABLE 19
Primer/Probe sets

| | | SEQ ID | |
|---|---|---|---|
| | | NO | Primer/probe |
| HSV-2 | HSV-2 US9 F | 287 | 5′ GGCAGAAGC |
| | | | CTACTACTCGGA |
| | | | AA 3′ |
| | HSV-2 US9 R | 133 | 5′ CCATGCGCA |
| | | | CGAGGAAGT 3′ |
| | HSV-2 US9 | 134 | 5′ 6-FAM-CGA |
| | probe | | GGCCGCCAAC- |
| | | | MGBNFQ 3′ |
| Guinea | gp GAPDH F | 135 | 5′ CATGACAAC |
| pig | | | TTCGGCATTGT |
| GAPDH | | | G 3′ |
| | gp GAPDH R | 136 | 5′ TCTTCTGGG |
| | | | TGGCAGTGAT |
| | | | G 3′ |
| | gp GAPDH | 137 | 5′ VIC-AGGGA |
| | probe | | CTCATGACCACA |
| | | | GT-MGBNFQ 3′ |

First, the master mix was prepared using the ratio shown in Table 20 below. 20 μL master mix was added to appropriate wells and stored covered at 4° C. until use.

TABLE 20
Ratio for master mix preparation

| Reagent | Vol/Rxn (μL) | Primer/Probe Mix Prep |
|---|---|---|
| Taq Man | 12.5 | Combine 60 μL each: forward |
| | | and reverse primers, probe + |
| Primer/Probe Mix | 1.5 | 1020 μL nuclease-free water. |
| Water | 6 | Make into ~160 μL aliquots; |
| Total Vol (μL) | 20 | store at −20° C. |

For preparation of the standard curve, the appropriate standard DNA aliquots were thawed. Five 0.6 mL tubes with nuclease-free water were prepared in the PCR laminar flow cabinet (see below for volume) and dilution series were completed for appropriate DNA type.

Then, 5 μL of test samples or standard concentration were added to the wells containing 20 μL of the Master Mix. The plate was covered with an optical film and centrifuged for 1 min at 1000 RPM (190 x g).

PCR procedure was performed on Applied Biosystems 7500 Fast PCR Machine with the following setup:
Holding state: step 1:95° C. 20 sec
Cycling state: step 1:95° C. 03 sec; step 2:60° C. 45 sec:
Number of cycles: 40.
Analyze results using Applied Biosystems' software

Limit of quantitation was established based on the total volume of media holding the swab, amount of swab media used for DNA isolation and the amount of DNA used for the PCR amplification. In this case, the total volume of swab media was approximately 1 mL, 200 μL was used to isolate DNA, that was resuspended in 200 μL, and 5 μL of DNA was used for DNA amplification. One copy of viral genome could be detected in this assay; therefore, the limit of quantitation was less than 200 HSV genome copy per swab.

Limit of detection was established based on the volume of DNA used per reaction. In this assay limit of detection was less than one HSV genome per amplification reaction.

### Statistical Analysis

GraphPad Prism 9 Software (La Jolla, USA) was used for statistical analysis and figure generation. As data did not pass test for normal distribution, Kruskal-Wallis-Test with uncorrected Dunn's test was performed for analysis of multiple data sets, and non-parametric Mann-Whitney for testing two data sets. For analysis of survival, log-rank (Mantel-Cox) test was performed.

### Results

### Immunogenicity

Serum and Vaginal IgG Titers after Second Immunization

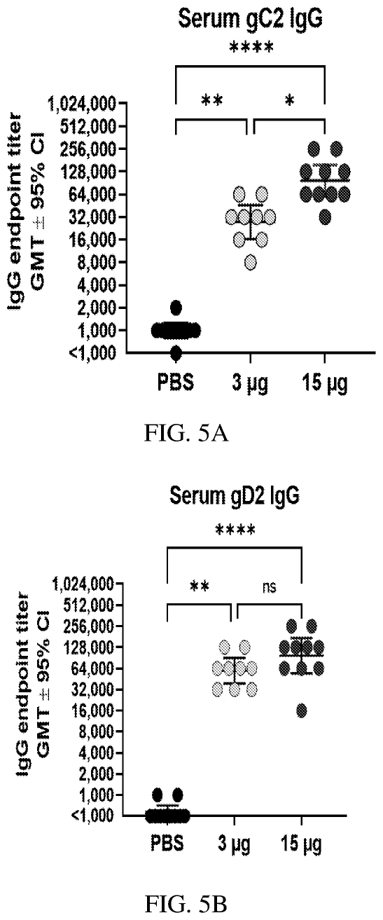

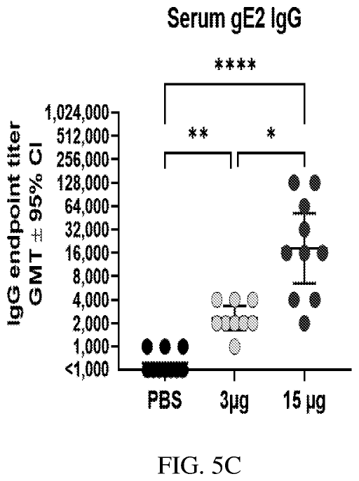

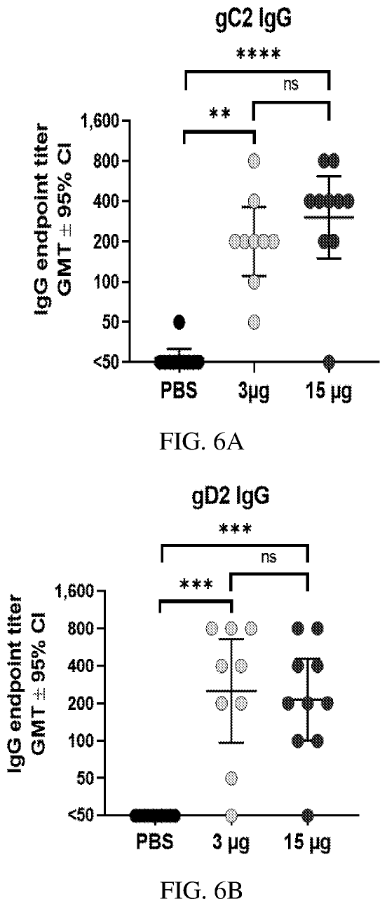

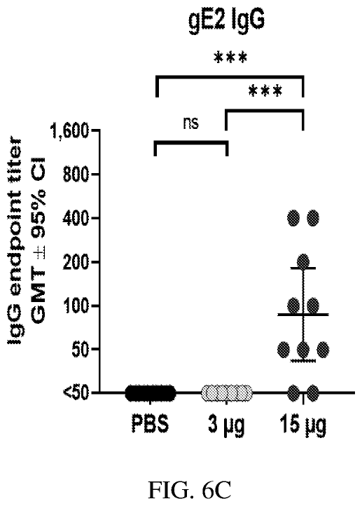

Serum and vaginal IgG antibodies against gC2, gD2, and gE2 were assessed by ELISA in samples obtained at day 56, 4 weeks after the second immunization. Results are shown in FIGS. 5 and 6.

The PBS control had no detectable serum or vaginal IgG antibodies. IgG titers could be detected for both doses for all three antigens with the exception of vaginal titers against gE2 at a 3 μg dose (gC2, serum 3 μg p: 0.0068 and 15 μg p: <0.0001, vaginal 3 μg p: 0.0018 and 15 μg p: <0.0001; gD2, serum 3 μg p: 0.0013 and 15 μg p: <0.0001, vaginal 3 μg p: 0.0003 and 15 μg p: 0.0007, gE2, serum 3 μg p: 0.0085 and 15 μg p: <0.0001, vaginal 15 μg p: 0.0001). A dose-response was observed between the 3 μg and 15 μg groups for gC2 serum titers, and gE2 serum and vaginal titers (gC2, serum p: 0.0420; gE2, serum p: 0.0409, vaginal p: 0.0002).

### Neutralizing Antibodies by Plaque Reduction Assay

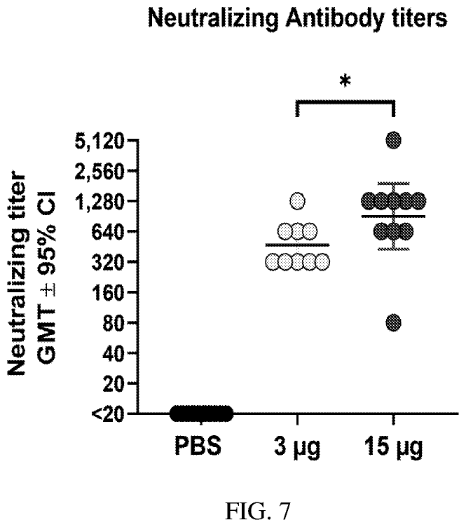

Virus-neutralizing antibodies in guinea pig serum were measured by plaque reduction assay using samples obtained one month after the second immunization. Both doses led to dose-dependent serum neutralizing antibody titers (FIG. 7; p: 0.0234).

### HSV-2 Challenge

At day 60, approximately one month after the second (final) immunization, animals in all groups were challenged with a lethal intravaginal dose of HSV-2 MS strain and scored for survival and signs of genital disease and vaginal virus titers for a period of up to 48 days.

### Weight Loss

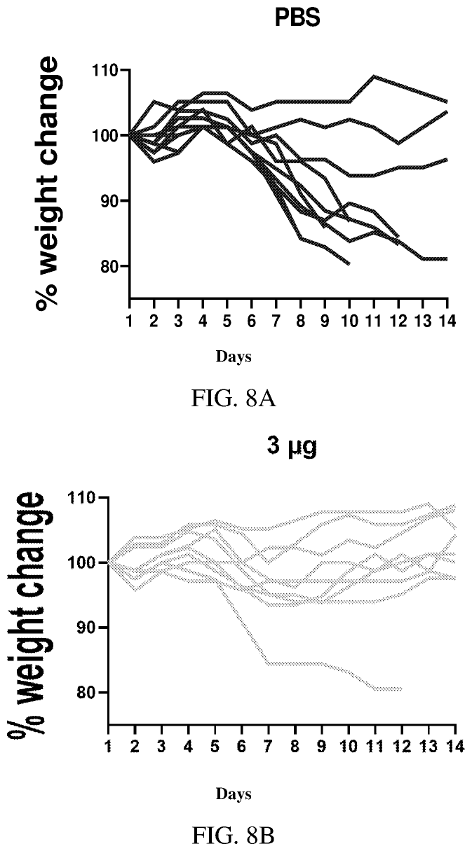

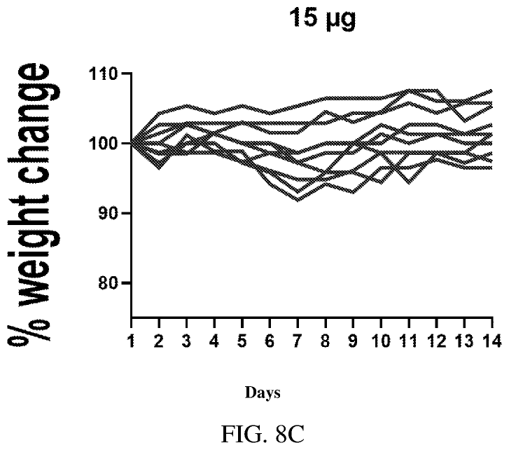

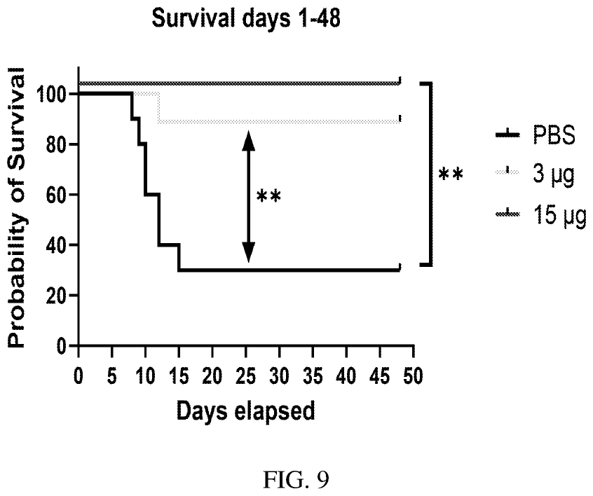

A comparable minor transient decrease in body weights was observed from day 5 post-challenge onwards for some animals in both groups receiving the HSV-2 modRNA vaccine after the first and second immunization (FIG. 8). Body weight loss was more evident and not transient for most animals in the PBS group and for one animal dosed with 3 μg of the modRNA vaccine.

### Survival

All animals dosed with 15 μg a combination comprising a polynucleotide survived viral challenge with HSV-2 MS strain at a dose of 25-fold LD₅₀, while one animal in the 3 μg dose group and seven animals in the PBS group succumbed to disease within 15 days (FIG. 9). Animals dosed with 3 μg or 15 μg showed a significantly higher probability of survival (3 μg, p: 0.0092; 15 μg, p: 0.0012).

### Disease Scores

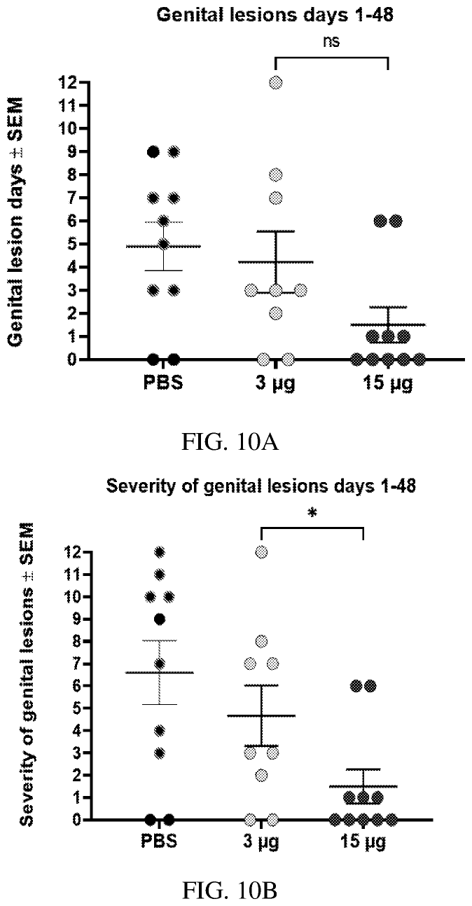

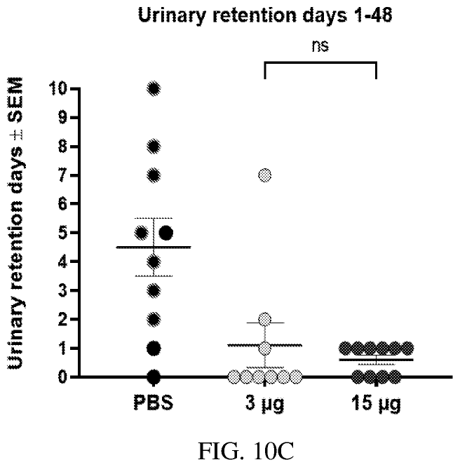

Disease severity, incidence, and duration of genital lesions as well as urinary retention were reduced in all immunized groups (FIG. 10). As most animals in the PBS group succumbed to viral challenge within the first 2 weeks after challenge (red bordered symbols), scoring genital lesions and urinary retention by days leads to an underrepresentation in disease scores in this group. Therefore, only immunized groups were included in the statistical analysis. Animals dosed with 15 μg showed a tendency towards decrease in days with genital lesion (FIG. 10(A), p: 0.0664) and a significant decrease in severity (FIG. 10(B), p: 0.0444) compared to 3 μg dosed animals, while no significant difference was detected in urinary retention (FIG. 10(C)).

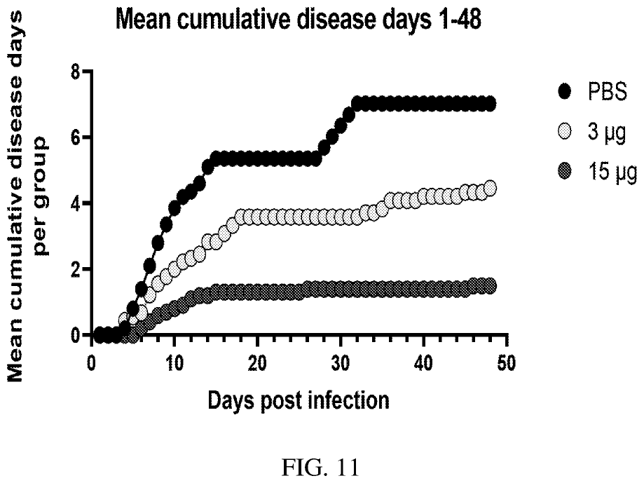

In addition to individual scores, mean cumulative days of disease up to 48 days after challenge were analyzed (FIG. 11). No scoring for days after death was assigned to animals that succumbed to viral disease. In addition to a reduction of days with disease directly after challenge, immunized animals showed a decrease in reoccurring genital disease over the course of 48 days.

### Vaginal Virus Titers

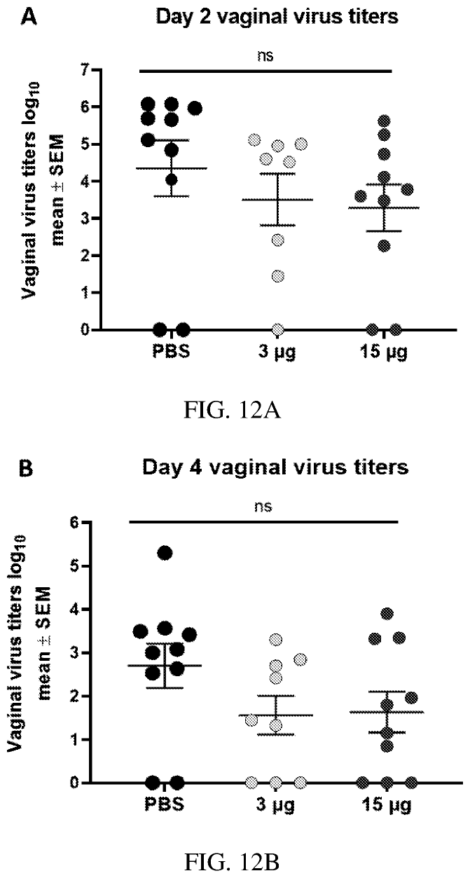

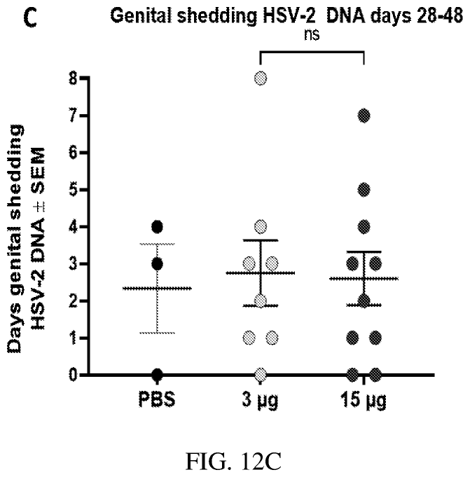

Two days and four days after viral challenge, vaginal virus titers were determined by plaque assay (FIG. 12). Although results were not significant, a clear tendency of reduction in vaginal virus titers was overserved in vaccinated groups for day 2 and day 4 compared to the PBS control. Appreciable differences were not apparent when comparing the 3 μg dose group with the 15 μg dose group on day 2 or 4, nor on mean days of shedding between day 28 to 48. Mean days of shedding of vaccinated animals were not compared to PBS controls, as a reasonable comparison was not possible since only three out of ten guinea pigs survived viral challenge.

### HSV-2 DNA Copy in DRG and Spinal Cord

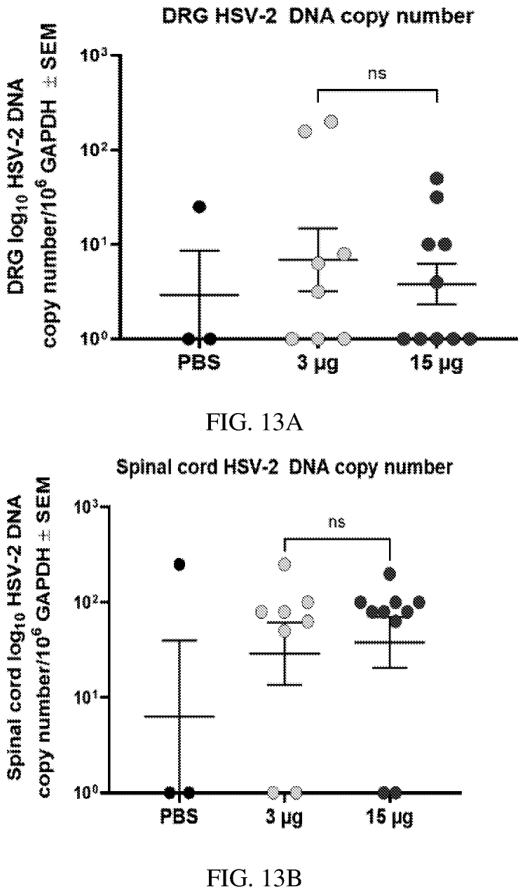

At day 48 (end of the study) post viral challenge, guinea pigs were euthanized and DRG (FIG. 13 (A)) and spinal cord tissue (FIG. 13 (B)) were collected. Both were analyzed for HSV-2 genomic copy number by quantitative PCR (qPCR) to assess latent infection. Separate reactions were used to amplify HSV-2 DNA and the GAPDH gene was used as control. HSV-2 DNA copy number was expressed as log 10 DNA copies per 106 GAPDH genes. HSV-2 DNA copy numbers of vaccinated animals were not compared to PBS controls, as a reasonable comparison was not possible because only 3 out of 10 guinea pigs survived viral challenge. No appreciable differences were apparent when comparing the 3 μg dose group with the 15 μg dose group regarding HSV-2 DNA copy numbers in DRG and spinal cord.

### CONCLUSION

The HSV-2 vaccine characterized in the present Example induced potent antigen-specific IgG antibodies in serum and vaginal mucosa, as well as high titers of neutralizing antibodies in serum. The clinical candidate provided protection against a highly lethal intravaginal challenge with HSV-2 in guinea pigs (for 90% at 3 μg dose and for 100% of animals at 15 μg dose), while only 30% of animals survived viral challenge in the control group. A significant dose-dependent response was shown in the induction of neutralizing antibody titers. The same tendency was observed with induction of serum and vaginal antibody responses upon immunization.

Furthermore, days and severity of genital lesions as well as urinary retention were reduced in immunized animals and vaginal titers tended to be decreased on day 2 and 4 after challenge compared to the unvaccinated control group. Overall, the results of the study support the HSV-2 vaccine as a clinical vaccine candidate.

### REFERENCES CITED IN EXAMPLE 5

Awasthi S, Hook L M, Shaw C E, Friedman H M. A trivalent subunit antigen glycoprotein vaccine as immunotherapy for genital herpes in the guinea pig genital infection model. Hum Vaccin Immunother. 2017 Dec. 2; 13 (12): 2785-2793
Awasthi S, Hook L M, Pardi N, Wang F, Myles A, Cancro M P, Cohen G H, Weissman D, Friedman H M. Nucleoside-modified mRNA encoding HSV-2 glycoproteins C, D, and E prevents clinical and subclinical genital herpes. Sci Immunol. 2019 Sep. 20.

### Example 6: In Vitro Testing of Further Polyribonucleotides Encoding HSV-2 Antigens

The present example demonstrates high transfection rates and expression levels in HEK293T cells transfected with modRNAs encoding gC, gD, or gE variants.

HEK293T cells were transfected with 0.2 μg/mL modRNA encoding gC2 or gD2 antigen constructs, or 0.4 μg/mL modRNA encoding gE2 antigen constructs, using a commercial transfection reagent. In the present example, the following modRNAs were transfected into HEK293T cells:
For the gC2 antigen: a modRNA comprising a ribonucleic acid sequence according to SEQ ID NO:106 (construct 1600) encoding a polypeptide that comprises an IL2 secretory signal and a gC2 antigen; a modRNA comprising a ribonucleic acid sequence according to SEQ ID NO: 204 (construct 2138) encoding a polypeptide that comprises a gE2 secretory signal and a gC2 antigen; a modRNA comprising a ribonucleic acid sequence according to SEQ ID NO: 192 (construct 2140) encoding a polypeptide that comprises a gD1 secretory signal and a gC2 antigen; a modRNA comprising a ribonucleic acid sequence according to SEQ ID NO: 196 (construct 2141) encoding a polypeptide that comprises a gB1 secretory signal and a gC2 antigen; or SEQ ID NO: 107 (construct 1876) encoding a polypeptide that comprises a IL2 secretory signal and a gC2 antigen;
for the gD2 antigen: a modRNA comprising a ribonucleic acid sequence according to SEQ ID NO: 118 (construct 1601) encoding a polypeptide that comprises a gD2 secretory signal and a gD2 antigen; a modRNA comprising a ribonucleic acid sequence according to SEQ ID NO: 117 (construct 1874) encoding a polypeptide that comprises a gD2 secretory signal and a gD2 antigen; a modRNA comprising a ribonucleic acid sequence according to SEQ ID NO: 120 (construct 1877) encoding a gD2 secretory signal and a gD2 antigen; or a modRNA comprising a ribonucleic acid sequence according to SEQ ID NO: 119 (construct 1659) encoding a gD2 secretory signal and a gD2 antigen; or
for the gE2 antigen: a modRNA comprising a ribonucleic acid sequence according to SEQ ID NO: 123 (construct 1602) encoding a polypeptide that comprises a IL2 secretory signal and a gE2 antigen; a modRNA comprising a ribonucleic acid sequence according to SEQ ID NO: 127 (construct 1660) encoding a polypeptide that comprises a gD2 secretory signal and a gE2 antigen; or a modRNA comprising a ribonucleic acid sequence according to SEQ ID NO: 212 (construct 2143) encoding a polypeptide that comprises a gD1 secretory signal and a gE2 antigen.

Briefly, 0.4×10⁶ HEK293T cells were seeded 6 h prior to transfection in 12-well plates. modRNA encoding for HSV-2 gC (gC2), gD (gD2) or gE (gE2) variants were formulated using Lipofectamine™ MessengerMAX™ (ThermoFisher Scientific) according to the manufacturers' instructions prior to transfection in triplicates. Cells were transfected with 0.2 μg/mL modRNA encoding gC2 and gD2 antigen constructs, or 0.4 μg/mL modRNA encoding gE2 antigen constructs. After transfection, cells were incubated to express the modRNA for 18 h at 37° C. and 5% CO₂ prior to staining. Afterwards, cells were harvested and incubated with a viability dye (eBioscience™ Fixable Viability Dye eFluor™ 450, ThermoFisher Scientific), fixed (Fixation Buffer, BioLegend), permeabilized (Permeabilization Buffer, eBioScience) and incubated with a mouse anti-gC2, anti-gD2 or anti-gE2 antibody and subsequently stained with an Alexa Fluor® 647 AffiniPure Donkey Anti-Mouse IgG (H+L) secondary antibody (Jackson ImmunoResearch) to detect expression of the antigens in HEK293T cells. The median fluorescence intensity (MFI) measured in FACS serves as a surrogate for antigen expression. Cells were acquired using a BD FACSCelesta 2 (BD) and data was analyzed with FlowJo V10.8 (BD).

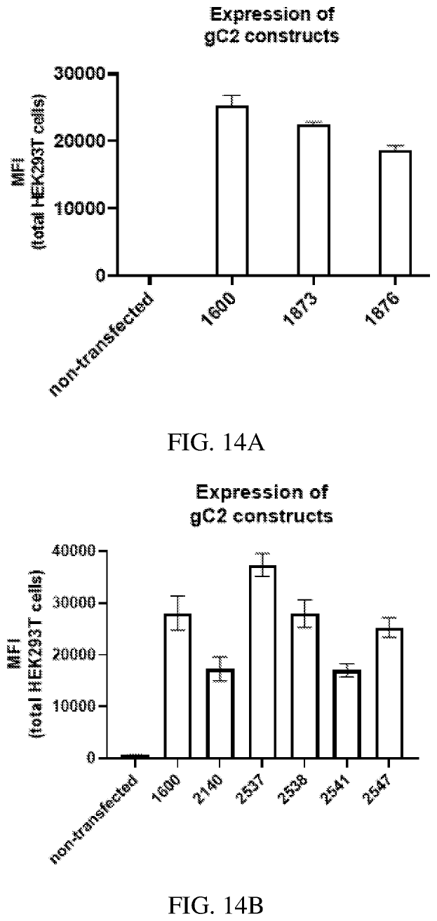

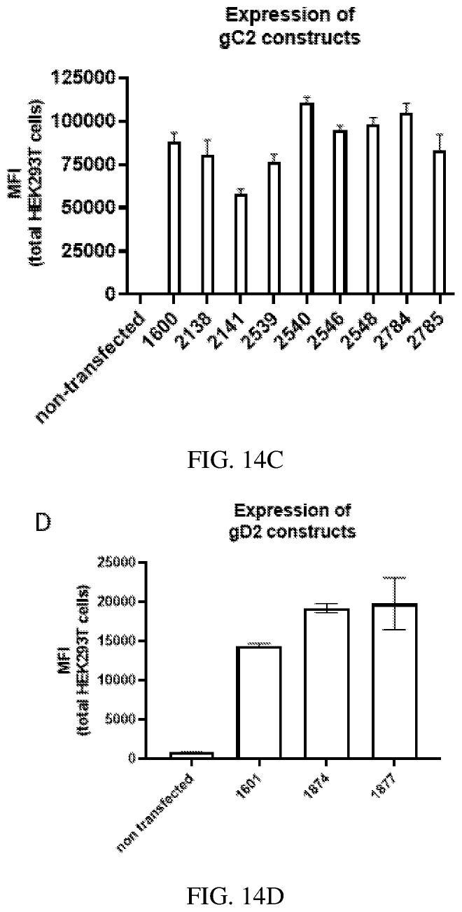

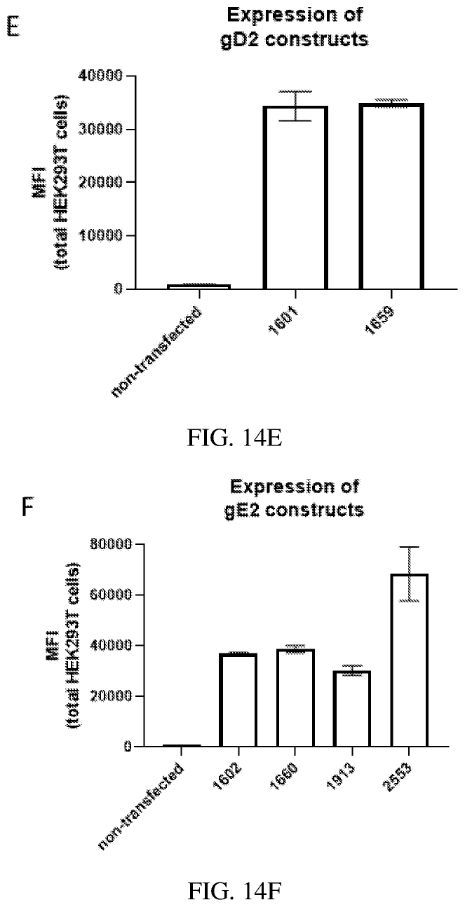

Median fluorescence intensity (MFI) of the total HEK293T population depicted per antigen is shown in FIGS. 14A, B, and C. Antigen variants showed comparable or enhanced expression (FIGS. 14D, E, and F) as compared to RNA comprising SEQ ID NOs: 106 (construct 1600), 118 (construct 1601), or 123 (construct 1602), which combined have been shown in Example 5 to induce a potent immune response in animal subjects. As in Example 5, these results indicate that the modRNA constructs characterized in the present Example would produce a strong immune response.

### Example 7: Example Clinical Studies of RNA Vaccine Compositions

The present Example describes certain clinical assessments that may be performed of certain Trivalent Vaccines described herein.

In some embodiments, more than one different Trivalent Vaccine candidate may be assessed. In some such embodiments, different candidates may vary, for example in:
(1) RNA platform (e.g., unmodified RNA, nucleoside-modified RNA, self-amplifying RNA (saRNA), trans-amplifying RNA);
(2) encoded antigen—e.g.,
which HSV (HSV-1 and/or HSV-2) protein(s) utilized
full length protein antigen vs fragment vs plurality of fragments vs fusion with one or more heterologous sequences (e.g., membrane tether, secretion, linker(s))
epitopes from different (and/or multiple) phases of HSV life cycle
(3) number of RNAs
(4) elements of RNA construct
cap and/or cap-adjacent sequences
5′ UTR
3′ UTR
polyA tail
(5) lipid composition of LNP.

In one embodiment, a candidate Trivalent Vaccine comprises three RNAs described herein: a first RNA encoding an HSV-2 gC antigen, a second RNA encoding an HSV-2 gD antigen, and a third RNA encoding an HSV-2 gE antigen. In this particular exemplary embodiment, vaccine candidates may be evaluated by intramuscular administration, for example, based on a dose-escalation scheme.

### Example 8: In vitro Testing of Further Polyribonucleotides Encoding HSV-2 Antigens

The present Example demonstrates high transfection rates in HEK293T cells of modRNAs encoding gC2 and gE2 variants as well as high expression levels in HEK293T cells of gC2, gD2, or gE2 variants.

HEK293T cells were transfected with 0.2 μg/mL modRNA encoding gC2, gE2 or gD2 antigen using a commercial transfection reagent. In the present example, the following modRNAs were transfected into HEK293T cells:
modRNAs comprising SEQ ID NO: 104 (construct 1597) or SEQ ID NO: 106 (construct 1600) encoding a gC2 antigen with an IL2 secretory signal;
modRNAs comprising SEQ ID NO: 116 (construct 1598), or SEQ ID NO: 118 (construct 1601) encoding a gD2 antigen with a gD2 secretory signal; or
modRNAs comprising SEQ ID NO: 121 (construct 1599) or SEQ ID NO: 123 (construct 1602) encoding a gE2 antigen with an IL2 secretory signal.

After transfection, cells were incubated to express the modRNA. Afterwards, cells were harvested and probed with antigen-specific mouse monoclonal antibodies and a secondary fluorescence-tagged anti-mouse antibody to detect expression of vaccine antigens in HEK293T cells. The median fluorescence intensity (MFI) measured in FACS serves as a surrogate for antigen expression.

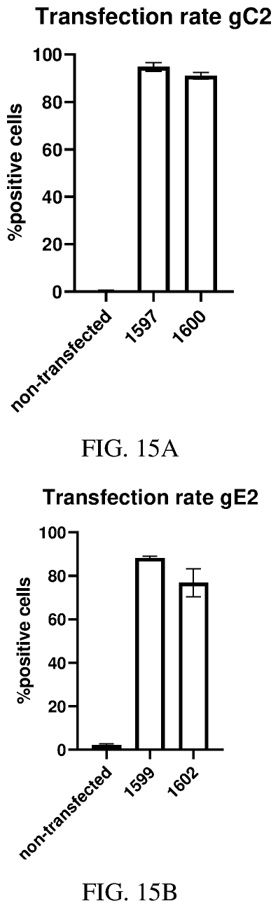

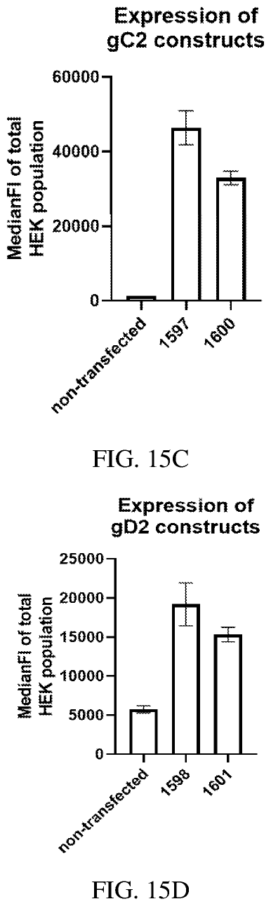

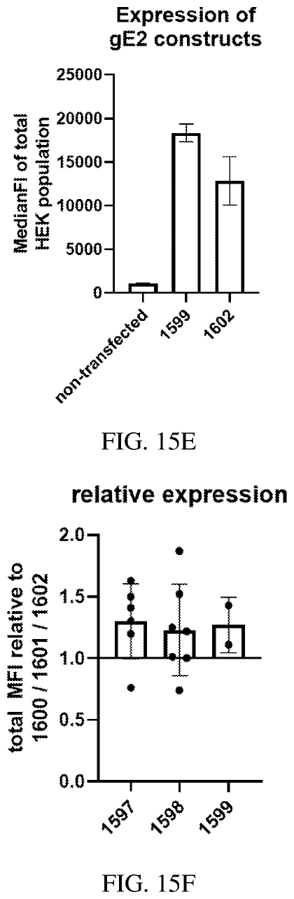

As shown in FIG. 15, gC and gE variants were expressed in HEK293T cells (FIG. 15 (A) and (B)). gC, gD, and gE showed comparable or enhanced protein expression (C, D, and E respectively) compared to gD variant SEQ ID NO: 118 (construct 1601) or gE variant SEQ ID NO: 123 (construct 1602), which have been shown in Example 5 to induce a potent immune response in animal subjects.

Cumulative total HEK293T expression data from up to n=7 (F) experiments are shown in relation to the gC2 (construct 1600, SEQ ID NO: 106), gD2 (construct 1601, SEQ ID NO: 118) or gE2 (construct 1602, SEQ ID NO: 123) constructs, respectively.

As in Example 5, these results indicate that the modRNA constructs characterized in the present Example would produce a strong immune response.

### Example 9: Exemplary Trivalent HSV-2 Vaccine Drug Product

The present example describes an exemplary HSV-2 Trivalent Vaccine, BNT163. BNT163 is a preservative-free, fixed dose combination, sterile suspension, for dilution. BNT163 contains three RNAs formulated as lipid nanoparticles (LNP), in an aqueous cryoprotectant buffer and is, after dilution with sterile normal saline, suitable for intramuscular (IM) injection. The total concentration of the three RNAs is 0.5 mg/mL. The LNP formulation can protect the RNA from extracellular RNases after administration. BNT163 may prevent and/or treat genital lesions and/or oral lesions caused by HSV-2 and/or HSV-1 through induction of immune responses to control the pathological effects induced by HSV infection and replication.

The composition of BNT163 is provided in Table 21.

TABLE 21
Composition of drug product.

| | | | Quantity |
|---|---|---|---|
| | | Concentration | per vial ¹ |
| Component | Function | (mg/mL) | (mg) |
| RBP027.1² | Active substance | 0.17 | 0.033 |
| RBP028.1² | Active substance | 0.17 | 0.033 |
| RBP029.1² | Active substance | 0.17 | 0.033 |
| ALC-0315 | Functional lipid | 7.17 | 1.434 |
| ALC-0159 | Functional lipid | 0.89 | 0.178 |
| DSPC | Structural lipid | 1.58 | 0.312 |
| Cholesterol | Structural lipid | 3.10 | 0.620 |
| Sucrose | Cryoprotectant | 102.69 | 20.538 |
| Sodium chloride | Buffer | 6.00 | 1.200 |
| Potassium chloride | Buffer | 0.15 | 0.030 |
| Disodium hydrogen | Buffer | 1.08 | 0.216 |
| phosphate dihydrate | | | |
| Potassium dihydrogen | Buffer | 0.15 | 0.030 |
| phosphate | | | |
| Water for Injection | Solvent/Vehicle | q.s. | q.s. |

¹ The quantity of each component is reported for the fill volume of 0.2 mL.
² The active substance is added as a 1:1:1 fixed combination with total of 0.5 mg/mL RNA. ALC-0315 = ((4-hydroxybutyl)azanediyl)bis(hexane-6,1-diyl)bis(2-hexyldecanoate). ALC-0159 = 2-[(polyethylene glycol)-2000]-N,N-ditetradecylacetamide. DSPC = 1,2-Distearoyl-sn-glycero-3-phosphocholine.
q.s. = quantum satis (as much as may suffice)

BNT163 is filled into a vial with a fill volume of 0.2 mL. BNT163 is stored frozen at −80 to −60° C. The primary container closure system consists of a glass vial closed with a rubber stopper and sealed with an aluminum cap with a plastic flip-off disk.

BNT163 is diluted, with sterile Normal Saline (0.9% sodium chloride) prior to administration.

### Example 10: Characterization of safety and immunogenicity of exemplary HSV Vaccine Candidates

### Objectives

The present example describes a Phase I randomized, observer-blinded, placebo-controlled, 2-part, dose escalation and expanded safety evaluation trial to evaluate the safety, tolerability, and immunogenicity of an exemplary Trivalent HSV2 vaccines, BNT163, for the prevention of genital lesions caused by HSV-2 and/or HSV-1.

This example includes two parts: Part A and Part B. Part A is a dose escalation part and Part B is an expanded safety evaluation and dose evaluation part. BNT163 investigational medicinal product (IMP) is shown in Table 22.

TABLE 22
Trial treatments

| | |
|---|---|
| IMP name | BNT163 |
| Type: | BNT163 polyribonucleotide-LNP vaccine (nucleoside-modified |
| | polyribonucleotide proportionally mixed 1:1:1 by weight of the gC2:gD2:gE2 |
| | polyribonucleotide respectively) or placebo (isotonic NaCl solution [0.9%]). |
| Route of | IM; Upper arm, musculus deltoideus. |
| administration: | |
| Dose levels: | Part A*: Three-dose regimen: |
| | 3 μg (1 μg of each polyribonucleotide) or placebo (active |
| | vaccine:placebo 5:1) |
| | 10 μg (3.33 μg of each polyribonucleotide) or placebo (active |
| | vaccine:placebo 5:1) |
| | 30 μg (10 μg of each polyribonucleotide) or placebo (active |
| | vaccine:placebo 5:1) |
| | 60 μg (20 μg of each polyribonucleotide) or placebo (active |
| | vaccine:placebo 5:1) |
| | Part B: Three-dose regimen: |
| | Two selected dose levels (≤60 μg) based on data from Part A (subjects |
| | randomized 1:1 to either dose level) |
| Dosing | Parts A and B: Three IM doses, one each at Visits 1, 4, and 7 (8 and 16 weeks |
| schedules: | after the previous dose, respectively). |

*Additional ”in-between” dose levels may be introduced based on the outcome of Internal Review Committee meetings.
Abbreviations: gC2, gD2, gE2 = glycoproteins C, D, and E from herpes simplex virus-2; IM = intramuscular; IMP = investigational medicinal product; RNA = ribonucleic acid; RNA-LNP = RNA-lipid nanoparticle.

Part A: Part A will focus on safety evaluations. Vaccine-induced immune responses (specifically neutralizing antibodies) will also be analyzed to assess if there is a dose-response.

Part A will enroll ~48 healthy subjects aged 18 to 55 years without current or history of symptomatic genital herpes infections. Each group of six subjects will be randomized observer-blinded, placebo controlled, 5:1 to vaccine: placebo. Part A will evaluate four planned dose levels (DLs): 3, 10, 30 and 60 μg. In each DL, subjects will receive three administrations of the BNT163 vaccine or placebo at Visit 1 (Day 0), Visit 4 (Day 56±2 from Visit 1) and Visit 7 (Day 112±5 of Visit 4).

Starting at 3 μg, the first two subjects will be dosed and complete 7 days (d) of follow-up post-Dose 1. If there is no trial treatment pausing, four more subjects will receive Dose 1. Once the first six subjects have completed the 7 d follow-up post-Dose 1, the following applies:
If there is no trial treatment pausing rule met in the first six subjects, six further subjects will be dosed at the same DL (thus, in total 12 subjects will be dosed in the respective DL).
If no safety findings of clinical concern are observed, dosing at 10 μg will be assessed.

Enrollment of subsequent DL cohorts, 10, 30 and 60 μg, will be conducted sequentially, e.g., with two subjects completing 7 d of follow-up post Dose 1, followed by enrollment of four more subjects.

Part B: Part B will enroll ~200 healthy subjects aged 18 to 55 years without current or history of symptomatic genital herpes infections. Part B of the trial will expand the safety characterization for two BNT163 dose levels (≤60 μg) selected based on Part A data and also enable a more comprehensive assessment of the impact of pre-existing immunity to HSV-1 and -2 on the safety and BNT163-induced immune responses than could be assessed in Part A.

Part B may be initiated once the last cohort of 12 subjects of Part A have completed 7 d of follow-up after receiving their second trial vaccination.

Part B will enroll ~200 subjects randomized 1:1 to one of two dose levels (≤60 μg) of BNT163 selected based on data from Part A. Randomization into Part B will be stratified by HSV 1/HSV 2 baseline serostatus, as follows:
1. HSV-1 negative/HSV-2 negative; 100 subjects, 50 in each of the two selected BNT163 dose groups.
OR
2. Any seropositive status (HSV 1 positive/HSV 2 positive or HSV 1 positive/HSV 2 negative or HSV-1 negative/HSV 2 positive); 100 subjects, 50 in each of the two selected BNT163 dose groups. Subjects of any seropositive status will be further stratified based on HSV-2 serostatus (HSV-1 positive/HSV-2 positive or HSV-1 negative/HSV-2 positive vs. HSV-1 positive/HSV-2 negative) with a minimum of 20 (maximum of 30) HSV-2 seropositive subjects and a minimum of 20 (maximum of 30) HSV-2 seronegative subjects per each of the selected BNT163 dose groups.

### Outcome Measures

Outcome measures for assessing the safety and tolerability of BNT163 in healthy adults during the dose escalating in Part A and during the expanded safety and dose evaluation in Part B include frequency of solicited local reactions (e.g., pain, erythema/redness, induration/swelling) at the injection site recorded up to 7 d after each dose, frequency of solicited systemic reactions (e.g., vomiting, diarrhea, headache, fatigue/tiredness, myalgia, arthralgia, chills, and fever) recorded up to 7 d after each dose, proportion of subjects with at least one unsolicited adverse event (AE) occurring up to 28 d after each dose, proportion of subjects in each cohort with at least one serious adverse event (SAE), or adverse events of special interest (AESI), or medically attended adverse event (MAAE) occurring up to 24 weeks post-Dose 3, and/or number and proportion of unsolicited AEs occurring up to 28 d after each dose.

Outcome measures for assessing immune response and changes in HSV-2 neutralizing and binding antibody titers from baseline for vaccine (Part A and Part B) and in relationship to placebo (Part A) include geometric mean titers (GMTs) at each time point, geometric mean fold (GMF) change from baseline of neutralizing and binding antibody titers to each time point after vaccination, and/or proportion of subjects with seroconversion defined as a minimum of 4-fold increase from baseline of neutralizing and binding antibody titers to each subsequent time point after vaccination.

An exploratory objective for Study A and Study B includes assessing the effects on pre-existing HSV-2 and/or HSV-1 serology status on immunogenic responses (e.g., neutralizing and binding antibody titers and/or number and proportion of subjects with seroconversion defined as a minimum of 4-fold increase of neutralizing antibody titers) per the following serology groups: (1) HSV-1−, HSV-2− subjects; (2) HSV-1+, HSV-2− subjects; (3) HSV-1±, HSV-2+subjects.

An additional exploratory objective for Study A and Study B includes assessing B- and T-cell responses (e.g., absolute and fold-change from baseline for CD4 and CD8 T- and B-cell responses to HSV-1 and HSV-2 gC2, gD2, and gE2; frequency and phenotypic characterization of cell mediated immune (CMI) responses such as CD4 and CD8 T- and B-cell responses to HSV-1 and HSV-2 gC, gD, and gE; and/or functional activity of the responses) to HSV antigen after BNT163 vaccination per different dose levels (DLs) and schedules).

An additional exploratory objective includes assessing cross-reactivity to HSV-1, including cross-binding of vaccine-induced antibodies to HSV-1 gD, gC, and gE, and cross-neutralization to HSV-1 (e.g., GMTs at each time point; GMF change from baseline of neutralizing and binding antibody titers to each time point after vaccination; and/or proportion of subjects with seroconversion defined as a minimum of 4-fold increase from baseline of neutralizing antibody titers to each subsequent time point after vaccination).

An additional exploratory objective includes assessing the number of breakthrough lesions/complications attributed to HSV infection (e.g., total number and type (oral vs. genital) of breakthrough lesions/complications reported until 28 d post-Dose 3).

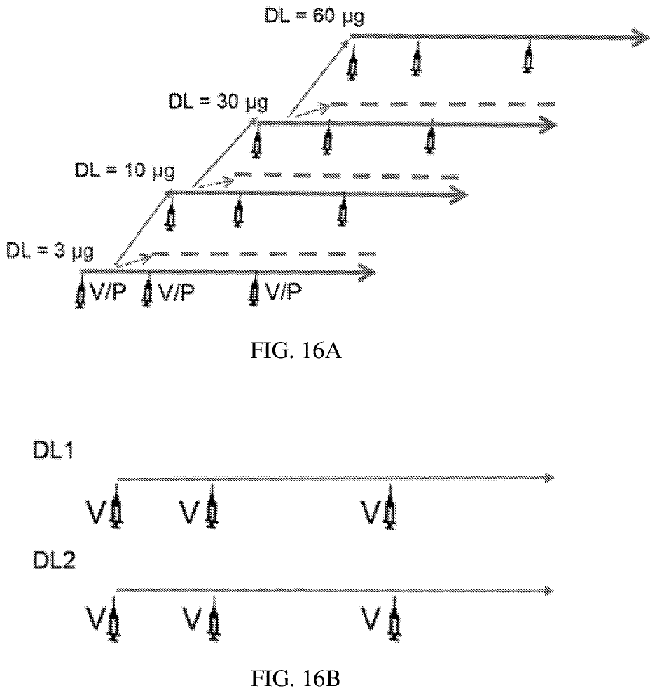

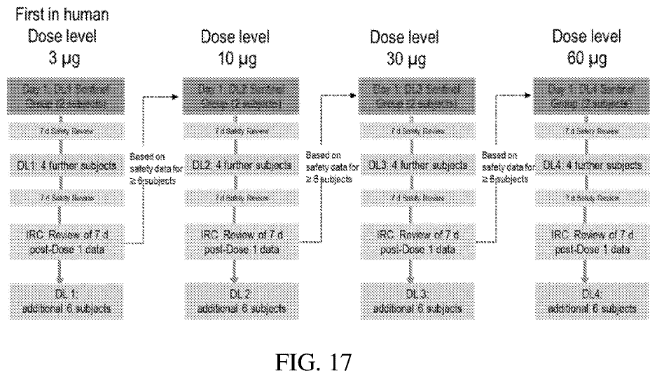

FIG. 16 and FIG. 17 are schematic diagrams of the clinical trial design. FIG. 16 describes a flow diagram of Part A and Part B. FIG. 17 describes the Part A dose escalation schema.

### Duration of all Trial Periods

The planned trial duration for each trial subject in Part A and Part B is ~80 weeks (~4 weeks screening, ~24 weeks treatment phase, and ~52 weeks follow-up phase). In total, the overall trial duration will be ~130 to ~150 weeks. The schedule of activities includes: Visit 0 (screening), Visit 1 (Dose 1), Visit 2 (7±3 days follow-up from Visit 1), Visit 3 (28±2 days follow-up from Visit 1), Visit 4 (56±2 days from Visit 1: Dose 2), Visit 5 (7 days follow-up from Visit 4), Visit 6 (28±2 days follow-up from Visit 4), Visit 7 (112±5 days from Visit 4: Dose 3), Visit 8 (7±3 days follow-up from Visit 7), Visit 9 (28±2 days follow-up from Visit 7), Visit (Call) 10 (90±10 days follow-up from Visit 7), Visit 11 (167±10 days follow-up from Visit 7), and Visit 12 (365±10 days follow-up from Visit 7).

### Trial Population

This study includes volunteers aged 18 to 55 years without current or history of symptomatic genital herpes infections. A balanced enrollment of males and females is not required.

### Number of Trial Subjects

A total of ~248 subjects are expected to enroll: ~48 subjects in Part A and ~200 subjects in Part B. In all cohorts from Part A, trial subjects who do not receive three investigational medicinal product (IMP) (BNT163) administrations for reasons other than (any kind of) safety concerns may be replaced (if that cohort is still open for randomization) to enable adequate assessment of BNT163 safety and immunogenicity.

### Inclusion Criteria

Volunteers are eligible to be included in the trial only if all of the following criteria apply:
1. Have given informed consent by signing and dating the informed consent form (ICF) before initiation of any trial-specific procedures.
2. Are aged 18 to 55 years, have a body mass index over 18.5 kg/m² and under 35 kg/m² and weigh at least 50 kg at Visit 0.
3. Are willing and able to comply with scheduled visits, treatment schedule, laboratory tests, and other requirements of the trial. This includes that they are able to understand and follow trial-related instructions.
4. Are overall healthy in the clinical judgment of the investigator based on medical history, physical examination, 12 lead electrocardiogram (ECG), vital signs, and screening laboratory tests (blood clinical laboratory) at Visit 0.

### Virology

5. Negative HIV-1 and -2 blood test: Sites may use locally available Clinical Laboratory Improvement Amendments (CLIA)-certified assays at Visit 0.
6. Negative Hepatitis B surface antigen at Visit 0.
7. Negative anti-Hepatitis C virus (HCV) antibodies (anti-HCV), or undetectable HCV viral load if the anti-HCV is positive at Visit 0.
8. Negative syphilis test at Visit 0.

### Reproductive Status and Contraception

9. Volunteers of childbearing potential (VOCBP): negative serum beta human chorionic gonadotropin (B-HCG) pregnancy test at Visit 0 and negative urine pregnancy test prior to each IMP administration and at end of the trial. Volunteers born female that are postmenopausal (verified by follicle stimulating hormone [FSH] level) or permanently sterilized (verified by medical records) will not be considered VOBCP.
10. VOCBP who agree to practice a highly effective form of contraception (for guidance on highly effective forms of contraception) and to require their male partners to use condoms with a spermicidal agent, starting at Visit 0 and continuously until 60 d after receiving the last trial treatment.
11. VOCBP who agree not to donate eggs (ova, oocytes) for the purposes of assisted reproduction during trial, starting at Visit 0 and continuously until 60 d after receiving the last trial treatment.
12. Men who are sexually active with a VOCBP and have not had a vasectomy who agree to use condoms with a spermicidal agent and to practice a highly effective form of contraception with their partners of childbearing potential (for guidance on highly effective forms of contraception) during the trial, starting at Visit 0 and continuously until 90 d after receiving the last trial treatment.
13. Men who are willing to refrain from sperm donation, starting at Visit 0 and continuously until 90 d after receiving the last trial treatment.

### Exclusion Criteria

Volunteers are not eligible to be included in the trial only if any of the following criteria apply:
1. Breastfeeding or intending to become pregnant within the projected duration of the trial starting with Visit 0 until 60 d after receiving the last trial treatment or intending to father children within the projected duration of the trial starting with Visit 0 until 90 d after receiving the last trial treatment.
2. Current or history of symptomatic genital herpes infections. Volunteers with oral herpes or herpetic whitlow will not be excluded.
3. Current or history of any form of ocular HSV infection or HSV-related central nervous system disease or complication.
4. History of any serious adverse reactions to vaccines or to vaccine components such as lipids, and including history of anaphylaxis and related symptoms such as hives, respiratory difficulty, angioedema, and/or abdominal pain.
5. Current or history of the following medical conditions:
   a. Uncontrolled or moderate or severe respiratory diseases (e.g., asthma, chronic obstructive pulmonary disease); symptoms of asthma severity as defined in the most recent National Asthma Education and Prevention Program Expert Panel report—e.g., exclude a volunteer who:
      i. Uses a short-acting rescue inhaler (typically a beta 2 agonist) daily, or
      ii. Uses high dose inhaled corticosteroids (per American Academy of Allergy Asthma & Immunology), or
      iii. In the past year has either of the following:
         1. Greater than one exacerbation of symptoms treated with oral/parenteral corticosteroids;
         2. Needed emergency care, urgent care, hospitalization, or intubation for asthma.
   b. History of thyroidectomy, or thyroid disease requiring medication during the last 12 months;
   c. History of diabetes mellitus type 1 or type 2, including cases controlled with diet alone (Not excluded: history of isolated gestational diabetes).
   d. Hypertension:
      i. If a person has been found to have elevated blood pressure or hypertension during screening or previously, exclude for blood pressure that is not well controlled. Well controlled blood pressure is defined as consistently ≤140 mm Hg systolic and ≤90 mm Hg diastolic, with or without medication, with only isolated, brief instances of higher readings, which must be ≤150 mm Hg systolic and ≤90 mm Hg diastolic at enrollment.
      ii. If a person does not have a history of elevated blood pressure or hypertension, also exclude for systolic blood pressure ≥150 mm Hg at enrollment or diastolic blood pressure ≥100 mm Hg confirmed by two measurements prior to enrollment.
   e. Malignancy within 5 years of Visit 0, excluding localized basal or squamous cell cancer;
   f. Current or history of cardiovascular diseases, e.g., myocardial infarction, congestive heart failure, cardiomyopathy, or clinically significant arrhythmias, myocarditis, or pericarditis;
   g. Bleeding disorder diagnosed by a doctor (e.g., factor deficiency, coagulopathy, or platelet disorder requiring special precautions);
   h. Seizure disorder: History of seizure(s) within past 3 years. Also exclude if volunteer has used medications in order to prevent or treat seizure(s) at any time within the past 3 years.
6. History of psychiatric illness, including alcohol abuse or drug addiction within 1 year before Visit 0, or a history (within the past 5 years) of substance abuse or known medical, psychological, or social conditions which, in the opinion of the investigator, could compromise their wellbeing if they participate as subjects in the trial, or that could prevent, limit, or confound the protocol specified assessments.
7. Any of the following associated with immune dysregulation:
   a. Primary immunodeficiencies.
   b. History of solid organ or bone marrow transplantation.
   c. Asplenia: any condition resulting in the absence of a functional spleen.
   d. Currently existing or history of autoimmune disease including and not limited to thyroid autoimmune disease, multiple sclerosis, psoriasis, etc.

### Prior/Concomitant Therapy

8. Use of any non-trial IMP within 28 d before Dose 1 in this trial (Visit 1) or planned receipt continuously until Visit 12 in this trial, or participation in the active treatment phase of another interventional clinical trial.
9. Previous vaccination with an investigational herpes virus vaccine at any time.
10. Any non-trial vaccination within 28 d before Dose 1 and continuously until 28 d after receiving Dose 3.
11. Received allergy treatment with antigen injections within 28 d before first IMP administration or that are scheduled within 14 d after Visit 1.
12. Received blood/plasma products or immunoglobulin within 120 d before Visit 1 or planned administration starting at Visit 0 until completion of Visit 12.
13. Received chronic suppressive antiviral therapy for treatment of recurrent HSV 1 and/or HSV 2 genital herpes infections (i.e., oral acyclovir, oral valacyclovir, oral famciclovir, and/or intravenous ganciclovir) from 1 year prior to Visit 0 until completion of Visit 12.

### Additional Exclusions

14. Any existing condition which may affect vaccine injection and/or assessment of local reactions assessment at the injection site, e.g., tattoos, severe scars.
15. Vulnerable individuals as per ICH E6 definition, i.e., are individuals whose willingness to volunteer in a clinical trial may be unduly influenced by the expectation, whether justified or not, of benefits associated with participation, or of a retaliatory response from senior members of a hierarchy in case of refusal to participate.
16. Any screening hematology and/or blood chemistry laboratory value that meets the definition of a Grade ≥2 abnormality at Visit 0. For laboratory values for which toxicity grading guidance is not available or for Grade≤1 abnormalities, subject eligibility will be determined at the discretion of the investigator.
   Part B only:
17. Applicable HSV serology stratum is already full.

### Concomitant Therapies During Trial

Study subjects should not receive prophylactic antipyretics and other pain medication to prevent symptoms associated with IMP administration. However, if a trial subject is taking a medication for another condition, even if it may have antipyretic or pain-relieving properties, it should not be withheld prior to IMP administration in this trial.

Administration of standard therapeutic dose of acetaminophen (preferable), or a non-steroidal anti-inflammatory drug (NSAID) if acetaminophen is contraindicated is permitted (paracetamol/acetaminophen at doses of up to 4 g/day).

Inhaled, topical, or localized administration of corticosteroids (e.g., intra-articular or intrabursal administration) are permitted.

The use of antipyretics and other pain medication to treat symptoms after IMP administration or for ongoing conditions is permitted, but not for prophylaxis (i.e., up to 12 h before each IMP administration).

Other concomitant medication may be considered on a case by case basis by the investigator, if required after consultation with the trial Medical Monitor.

### Assessments

HSV 1/HSV 2 serology is assessed by commercial HSV type-specific antibody tests and a Western Blot test to detect antibody responses to HSV 1/HSV 2 specific viral proteins.

Perceived pain at the injection site is assessed as absent, mild, moderate or severe, according to the grading scale in Table 23.

Subjects are provided with a ruler to measure erythema/redness and induration/swelling. Erythema/redness and induration/swelling are measured at the greatest single diameter and recorded and then categorized during analysis as absent, mild, moderate or severe, based on the grading scale in Table 23.

TABLE 23
Local Reaction Grading Scale

| | Mild | Moderate | Severe | Potentially life-threatening |
|---|---|---|---|---|
| | (Grade 1) | (Grade 2) | (Grade 3) | (Grade 4) ^{c} |
| Pain at the | Does not | Interferes with | Prevents daily | Emergency room visit or |
| injection | interfere with | activity | activity | hospitalization for severe |
| site | activity | | | pain |
| Erythema / | 2.5 cm to | >5.0 cm to | >10 cm | Necrosis or exfoliative |
| redness ^{a} | 5.0 cm | 10.0 cm | | dermatitis - investigator |
| | | | | or medically qualified |
| | | | | person confirmation is |
| | | | | required |
| Induration / | 2.5 cm to | >5.0 cm to | >10 cm | Necrosis - investigator or |
| swelling ^{b} | 5.0 cm | 10.0 cm | | medically qualified person |
| | | | | confirmation is required |

^{a} In addition to grading the measured local reaction at the greatest single diameter, the measurement should be recorded as a continuous variable. Erythema/redness should be measured using an appropriate measuring device provided by the sponsor.
^{b} Induration/swelling should be measured using an appropriate measuring device provided by the sponsor.
^{c} Investigator or medically qualified person confirmation is required for all reactogenicity graded as Grade 4.
Modified from the US FDA guidance (US FDA 2007)

Symptoms of systemic reactions are assessed as absent, mild, moderate, or severe, according to the grading scale in Table 24.

TABLE 24
Systemic reaction grading scale

| | Mild | Moderate | Severe | Potentially life-threatening |
|---|---|---|---|---|
| | (Grade 1) | (Grade 2) | (Grade 3) | (Grade 4) ^{a} |
| Fever (oral | 38.0° C./100.4° F. to | 38.5° C./101.2° F. to | 39.0° C./102.1° F. to | >40.0° C./>104.0° F. - |
| temperature | 38.4° C./101.1° F. | 38.9° C./102.0° F. | 40.0° C./104.0° F. | investigator or medically |
| of ≥38.0° | | | | qualified person |
| C. / ≥100.4° F.) | | | | confirmation is required |
| Vomiting | 1 to 2 times in | >2 times in 24 h | Requires intravenous | Emergency room visit or |
| | 24 h | | hydration | hospitalization for |
| | | | | hypotensive shock |
| Diarrhea | 2 to 3 loose | 4 to 5 loose stools | 6 or more loose stools | Emergency room visit or |
| | stools in 24 h | in 24 h | in 24 h | hospitalization for |
| | | | | severe diarrhea |
| Headache | Does not | Some interference | Prevents daily routine | Emergency room visit or |
| | interfere with | with activity | activity | hospitalization for |
| | activity | | | severe headache |
| Fatigue / | Does not | Some interference | Prevents daily routine | Emergency room visit or |
| tiredness | interfere with | with activity | activity | hospitalization for |
| | activity | | | severe fatigue |
| Chills | Does not | Some interference | Prevents daily routine | Emergency room visit or |
| | interfere with | with activity | activity | hospitalization for |
| | activity | | | severe chills |
| New or | Does not | Some interference | Prevents daily routine | Emergency room visit or |
| worsened | interfere with | with activity | activity | hospitalization for |
| muscle pain | activity | | | severe new or worsened |
| | | | | muscle pain |
| New or | Does not | Some interference | Prevents daily routine | Emergency room visit or |
| worsening | interfere with | with activity | activity | hospitalization for |
| joint pain | activity | | | severe new or worsened |
| | | | | joint pain |

^{a} Investigator or medically qualified person confirmation is required for all reactogenicity graded as Grade 4. Grade 4 reactions will be documented in the EDC system only and not in the subject's e-diary.
Abbreviations: C = Celsius; EDC = electronic data capture; e-diary = electronic diary; F = Fahrenheit; h = hours.
Modified from the US FDA guidance (US FDA 2007).

### Immune Responses

Immune responses are assessed at visit 0, 2, 5 and 8. Immune response analyses are categorized as neutralization and binding antibody titers, cell mediated immune (CMI) responses, and explorative research.

### Neutralization and Binding Antibody Titers

Neutralizing antibody titers and binding antibody titers assessments to HSV comprise of:
A functional antibody titer, e.g., viral neutralization test or an equivalent assay (e.g., pseudo-viral neutralization test assay).
Seronegative is defined as titers below the starting dilution (i.e., below the limit of detection for the assay).
Seroconversion after immunization is defined as a 4-fold increase in titer.
   For seronegative pre-immunization sera: a titer which is ≥4-fold the limit of detection.
   For seropositive pre-immunization sera: a titer which is 4-fold the measured pre-immunization titer.
An antibody binding assay, e.g., ELISA or an equivalent assay.
   For seronegative pre-immunization sera: a titer which is ≥4-fold the limit of detection.
   For seropositive pre-immunization sera: a titer which is 4-fold the measured pre-immunization titer.

### Cell Mediated Immune (CMI) Responses

Blood samples will be used to describe CMI responses to HSV 1 or HSV 2 and reference strains:
T cell responses include responses mediated by immune cells such as CD4 and CD8 T cells and their functional phenotypic subset by, e.g., enzyme-linked immunospot, intracellular cytokine staining, multimer analyses, cytokine secretion assays, flow cytometry, and other tests.
These analyses may further include CD4 and CD8 T cells, Th1-specific cytokines (e.g., interferon-gamma, tumor necrosis factor-alpha, interleukin [IL]-2, or IL-12) and Th2-specific cytokines (e.g., IL-4, IL-5, IL-10, IL-13) to analyze the induction of either balanced Th1/Th2 responses, or of unbalanced Th1-dominant or Th2-dominant immune responses, respectively.
T cell and B cell analyses may include characterization of vaccine induced plasmablasts and HSV antigen-specific B cells and T cells to identify B cells and T cells recognizing conserved and strain-specific epitopes which could include but is not limited to protein expression profile, gene expression or transcriptomic profile, epigenomic or open chromatin region profiling or metabolomic profiling. Further single cell variability diversity joining analysis and somatic hyper-mutation analysis may also be performed on B cells, and repertoire analysis on T cells.
Some of the sample may be used for sequencing of trial subjects' antibody and/or B-cell receptor heavy- and light-chain genes, T-cell receptor genes, and/or gene expression and/or epigenetic profile, for further understanding the B-cell, T-cell, and antibody repertoires and for generating monoclonal antibodies for product development or for development of T cell-based therapies.

### Additional Characterizations

Further exploratory research analyses may be conducted using residual biological samples from subsets of subjects in order to further characterize the vaccine induced and/or innate immune responses, e.g., phenotypic or functional characterization of antigen-specific B cells and T cells (e.g., by flow cytometry-based phenotyping including multimer staining), analysis of B-cell receptor/T-cell receptor repertoire (e.g., by next generation sequencing, single cell RNA sequencing), multiplex-cytokine, and inflammatory protein analysis. Transcriptomic, epigenomic, metabolomic, or phenotypic or functional characterization of other immune cell populations (e.g., innate immune cells) that may be relevant to understand the vaccine-induced immune responses or for the product development for BNT163 or related therapeutic areas may be included in this research

### Adverse Events

An adverse event (AE) is any untoward medical occurrence in a trial subject administered a pharmaceutical product and which does not necessarily have to have a causal relationship with this treatment. An AE can therefore be any unfavorable and unintended sign (including an abnormal laboratory finding), symptom, or disease (new or exacerbated) temporally associated with the use of a medicinal product, whether or not considered related to the medicinal product. A medically attended adverse event (MAAE) is an unsolicited AE for which the subjects received medical attention defined as hospitalization, or an otherwise unscheduled visit to or from medical personnel for any reason, including emergency room visits.

Events meeting the AE definition:
Any abnormal laboratory test results (e.g., hematology and clinical chemistry) or other safety assessments (e.g., ECG, vital signs measurements), including those that worsen from baseline, considered clinically significant in the medical and scientific judgment of the investigator.
Exacerbation of a chronic or intermittent pre-existing condition including either an increase in frequency and/or severity of the condition.
Serious adverse event (SAE) and non-serious trial procedure-related AEs occurring after the date informed consent was given (ICF signed and dated) and AEs (non-serious AEs) occurring after first IMP administration.
Signs, symptoms, or the clinical sequelae of a suspected drug-drug interaction.
Signs, symptoms, or the clinical sequelae of a suspected overdose of either trial treatment or a concomitant medication. Overdose per se will not be reported as an AE/SAE (for actions in case of overdose or errors in drug administration, see Section 8.4).

Events not meeting the AE definition:
Any clinically significant abnormal laboratory findings or other abnormal safety assessments which are associated with the underlying disease, unless judged by the investigator to be more severe than expected for the trial subject's condition.
Medical or surgical procedure (e.g., endoscopy, appendectomy): the condition that leads to the procedure is the AE.
Situations in which an untoward medical occurrence did not occur or continue (social and/or convenience admission to a hospital).

### Serious Adverse Events

A serious adverse event (SAE) is defined as any untoward medical occurrence that, at any dose, results in death or is life threatening.

The assessment of AE and/or SAE intensity is consistently done for all trial subjects treated with the same treatment and dose. For further guidance on AE and SAE assessments, see, for example US FDA 2007 guidance, incorporated herein by reference in its entirety. Where specific guidance for an AE term is not provided, the following general approach is followed:
Grade 1—Mild; does not interfere with the trial subject's usual function;
Grade 2—Moderate; interferes to some extent with the trial subject's usual function;
Grade 3—Severe; interferes significantly with the trial subject's usual function; and
Grade 4—Potentially life-threatening; life-threatening consequences, urgent intervention required.

### Example 11: Expression levels of exemplary HSV Vaccine Candidates

The present example demonstrates expression levels in HEK293 cells transfected with modRNAs encoding gC, gD, or gE.

HEK293T cells were transfected with 0.2 μg/mL modRNA encoding gC2 or gD2 antigen constructs, or 0.4 μg/mL modRNA encoding gE2 antigen constructs, using a commercial transfection reagent. In the present example, the following modRNAs were transfected into HEK293T cells:
For the gC2 antigen:
   a modRNA comprising a ribonucleic acid sequence according to SEQ ID NO:106 (construct 1600) encoding a polypeptide that comprises an IL2 secretory signal and a gC2 antigen (version 2);
   a modRNA comprising a ribonucleic acid sequence according to SEQ ID NO: 318 (construct 2787) encoding a polypeptide that comprises a gD2 secretory signal and a gC2 antigen (version 2);
   a modRNA comprising a ribonucleic acid sequence according to SEQ ID NO: 195 (construct 2542) encoding a polypeptide that comprises a gB1 secretory signal and a gC2 antigen (version 3); or
   a modRNA comprising a ribonucleic acid sequence according to SEQ ID NO: 319 (construct 2786) encoding a polypeptide that comprises a gC2 secretory signal and a gC2 antigen (version 2).
For the gE2 antigen:
   a modRNA comprising a ribonucleic acid sequence according to SEQ ID NO: 123 (construct 1602) encoding a polypeptide that comprises a IL2 secretory signal and a gE2 antigen (version 2);
   a modRNA comprising a ribonucleic acid sequence according to SEQ ID NO: 126 (construct 1911) encoding a polypeptide that comprises a gD2 secretory signal and a gE2 antigen (version 1);
   a modRNA comprising a ribonucleic acid sequence according to SEQ ID NO: 212 (construct 2143) encoding a polypeptide that comprises a gD1 secretory signal and a gE2 antigen (version 4);
   a modRNA comprising a ribonucleic acid sequence according to SEQ ID NO: 209 (construct 2552) encoding a polypeptide that comprises a gD1 secretory signal and a gE2 antigen (version 1);
   modRNA comprising a ribonucleic acid sequence according to SEQ ID NO: 211 (construct 2554) encoding a polypeptide that comprises a gD1 secretory signal and a gE2 antigen (version 3);
   modRNA comprising a ribonucleic acid sequence according to SEQ ID NO: 323 (construct 2788) encoding a polypeptide that comprises a gE2 secretory signal and a gE2 antigen (version 2);
   modRNA comprising a ribonucleic acid sequence according to SEQ ID NO: 322 (construct 2790) encoding a polypeptide that comprises a gE2 secretory signal and a gE2 antigen (version 2);
   modRNA comprising a ribonucleic acid sequence according to SEQ ID NO: 325 (construct 2791) encoding a polypeptide that comprises a gE2 secretory signal and a gE2 antigen (version 2); or
   modRNA comprising a ribonucleic acid sequence according to SEQ ID NO: 324 (construct 2792) encoding a polypeptide that comprises a gE2 secretory signal and a gE2 antigen (version 2).

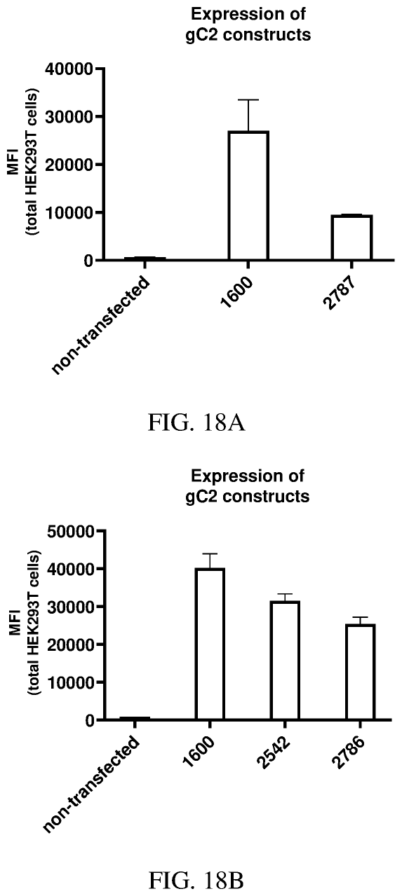

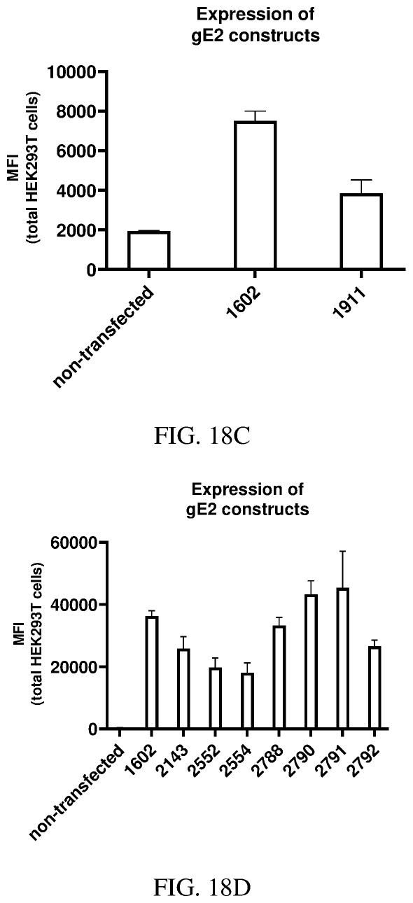

Expression of gC2, gD2 and gE2 proteins was detected by flow cytometry using primary monoclonal mouse antibodies detecting the respective antigen and a secondary fluorescent tagged anti-mouse antibody. Median fluorescence intensities (MFI) of the total HEK293T population for gC2 antigen constructs are shown in FIGS. 18A-B and for gE2 antigen constructs in FIGS. 18C-D. Strong expression was observed with gC2, gD2 and gE2 transfections for all constructs.

### Example 12: Secretion levels of exemplary HSV Vaccine Candidates

The present example demonstrates high secretion levels in HEK293 cells transfected with modRNAs encoding gC, gD, or gE.

HEK293T cells were transfected with 0.2 μg/mL modRNA encoding gC2 or gD2 antigen constructs, or 0.4 μg/mL modRNA encoding gE2 antigen constructs, using a commercial transfection reagent. In the present example, the following modRNAs were transfected into the HEK293T cells:
For the gC2 antigen:
   a modRNA comprising a ribonucleic acid sequence according to SEQ ID NO:106 (construct 1600) encoding a polypeptide that comprises an IL2 secretory signal and a gC2 antigen (version 2);
   a modRNA comprising a ribonucleic acid sequence according to SEQ ID NO: 105 (construct 1873) encoding a polypeptide that comprises an IL2 secretory signal and a gC2 antigen (version 1);
   a modRNA comprising a ribonucleic acid sequence according to SEQ ID NO: 107 (construct 1876) encoding a polypeptide that comprises an IL2 secretory signal and a gC2 antigen (version 2);
   a modRNA comprising a ribonucleic acid sequence according to SEQ ID NO: 204 (construct 2138) encoding a polypeptide that comprises a gE2 secretory signal and a gC2 antigen (version 4);
   a modRNA comprising a ribonucleic acid sequence according to SEQ ID NO: 194 (construct 2140) encoding a polypeptide that comprises a gD1 secretory signal and a gC2 antigen (version 2);
   a modRNA comprising a ribonucleic acid sequence according to SEQ ID NO: 196 (construct 2141) encoding a polypeptide that comprises a gB1 secretory signal and a gC2 antigen (version 2);
   a modRNA comprising a ribonucleic acid sequence according to SEQ ID NO: 189 (construct 2537) encoding a polypeptide that comprises a gD1 secretory signal and a gC2 antigen (version 1);
   a modRNA comprising a ribonucleic acid sequence according to SEQ ID NO: 190 (construct 2538) encoding a polypeptide that comprises a gD1 secretory signal and a gC2 antigen (version 2);
   a modRNA comprising a ribonucleic acid sequence according to SEQ ID NO: 191 (construct 2539) encoding a polypeptide that comprises a gD1 secretory signal and a gC2 antigen (version 3);
   a modRNA comprising a ribonucleic acid sequence according to SEQ ID NO: 193 (construct 2540) encoding a polypeptide that comprises a gB1 secretory signal and a gC2 antigen (version 1);
   a modRNA comprising a ribonucleic acid sequence according to SEQ ID NO: 194 (construct 2541) encoding a polypeptide that comprises a gB1 secretory signal and a gC2 antigen (version 2);
   a modRNA comprising a ribonucleic acid sequence according to SEQ ID NO: 195 (construct 2542) encoding a polypeptide that comprises a gB1 secretory signal and a gC2 antigen (version 3);
   a modRNA comprising a ribonucleic acid sequence according to SEQ ID NO: 201 (construct 2546) encoding a polypeptide that comprises a gE2 secretory signal and a gC2 antigen (version 1);
   a modRNA comprising a ribonucleic acid sequence according to SEQ ID NO: 202 (construct 2547) encoding a polypeptide that comprises a gE2 secretory signal and a gC2 antigen (version 2);
   a modRNA comprising a ribonucleic acid sequence according to SEQ ID NO: 203 (construct 2548) encoding a polypeptide that comprises a gE2 secretory signal and a gC2 antigen (version 3);
   a modRNA comprising a ribonucleic acid sequence according to SEQ ID NO: 321 (construct 2784) encoding a polypeptide that comprises a gC2 secretory signal and a gC2 antigen (version 2);
   a modRNA comprising a ribonucleic acid sequence according to SEQ ID NO: 320 (construct 2785) encoding a polypeptide that comprises a gC2 secretory signal and a gC2 antigen (version 2);
   a modRNA comprising a ribonucleic acid sequence according to SEQ ID NO: 319 (construct 2786) encoding a polypeptide that comprises a gC2 secretory signal and a gC2 antigen (version 2);
   a modRNA comprising a ribonucleic acid sequence according to SEQ ID NO: 318 (construct 2787) encoding a polypeptide that comprises a gD2 secretory signal and a gC2 antigen (version 2); or
   a modRNA comprising a ribonucleic acid sequence according to SEQ ID NO: 349 (construct 3233) encoding a polypeptide that comprises a gD1 secretory signal and a gC2 antigen (version 2).
For the gD2 antigen:
   a modRNA comprising a ribonucleic acid sequence according to SEQ ID NO: 118 (construct 1601) encoding a polypeptide that comprises an gD2 secretory signal and a gD2 antigen (version 2);
   a modRNA comprising a ribonucleic acid sequence according to SEQ ID NO: 119 (construct 1659) encoding a polypeptide that comprises a gD2 secretory signal and a gD2 antigen (version 2); or
   a modRNA comprising a ribonucleic acid sequence according to SEQ ID NO: 352 (construct 3234) encoding a polypeptide that comprises a gD2 secretory signal and a gD2 antigen (version 2).
For the gE2 antigen:
   a modRNA comprising a ribonucleic acid sequence according to SEQ ID NO: 123 (construct 1602) encoding a polypeptide that comprises a IL2 secretory signal and a gE2 antigen (version 2);
   a modRNA comprising a ribonucleic acid sequence according to SEQ ID NO: 126 (construct 1911) encoding a polypeptide that comprises a gD2 secretory signal and a gE2 antigen (version 1);
   a modRNA comprising a ribonucleic acid sequence according to SEQ ID NO: 127 (construct 1660) encoding a polypeptide that comprises a gD2 secretory signal and a gE2 antigen (version 2);
   a modRNA comprising a ribonucleic acid sequence according to SEQ ID NO: 130 (construct 1913) encoding a polypeptide that comprises a gE2 secretory signal and a gE2 antigen (version 2);
   modRNA comprising a ribonucleic acid sequence according to SEQ ID NO: 212 (construct 2143) encoding a polypeptide that comprises a gD1 secretory signal and a gE2 antigen (version 4);
   modRNA comprising a ribonucleic acid sequence according to SEQ ID NO: 209 (construct 2552) encoding a polypeptide that comprises a gD1 secretory signal and a gE2 antigen (version 1);
   modRNA comprising a ribonucleic acid sequence according to SEQ ID NO: 210 (construct 2553) encoding a polypeptide that comprises a gD1 secretory signal and a gE2 antigen (version 2);
   modRNA comprising a ribonucleic acid sequence according to SEQ ID NO: 211 (construct 2554) encoding a polypeptide that comprises a gD1secretory signal and a gE2 antigen (version 3);
   modRNA comprising a ribonucleic acid sequence according to SEQ ID NO: 323 (construct 2788) encoding a polypeptide that comprises a gE2 secretory signal and a gE2 antigen (version 2);
   modRNA comprising a ribonucleic acid sequence according to SEQ ID NO: 322 (construct 2790) encoding a polypeptide that comprises a gE2 secretory signal and a gE2 antigen (version 2);
   modRNA comprising a ribonucleic acid sequence according to SEQ ID NO: 325 (construct 2791) encoding a polypeptide that comprises a gE2 secretory signal and a gE2 antigen (version 2);
   modRNA comprising a ribonucleic acid sequence according to SEQ ID NO: 324 (construct 2792) encoding a polypeptide that comprises a gE2 secretory signal and a gE2 antigen (version 2); or
   modRNA comprising a ribonucleic acid sequence according to SEQ ID NO: 353 (construct 3235) encoding a polypeptide that comprises a gD2 secretory signal and a gE2 antigen (version 2).

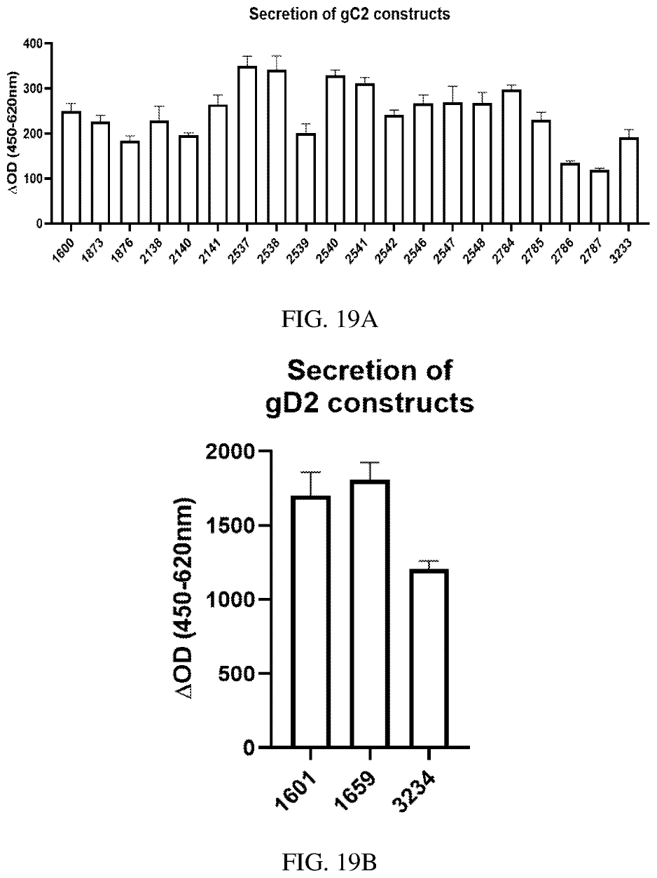

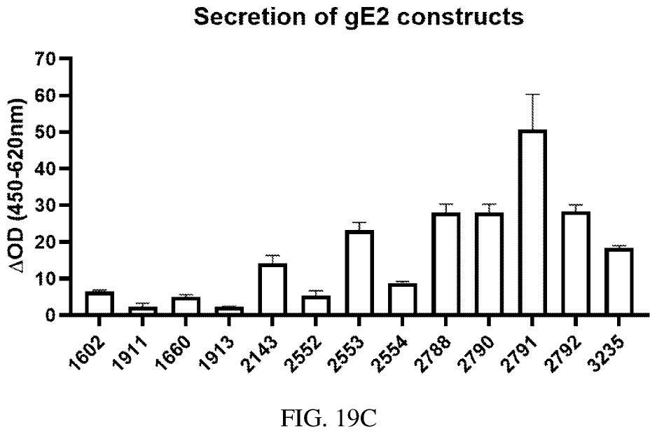

Secretion of gC2, gD2 and gE2 antigens was detected in the cell culture supernatants by ELISA using antibodies detecting the respective antigen. Representative data from one experiment showing AOD (450-620 nm) values for gC2 antigen constructs (FIG. 19A), for gD2 antigen constructs (FIG. 19B), and for gE2 antigen constructs (FIG. 19C). Data shown are mean±SD of HEK293T transfected cell culture supernatants performed in triplicates.

### NUMBERED EMBODIMENTS

1. A polyribonucleotide encoding a polypeptide, wherein the polypeptide comprises an HSV glycoprotein C (gC) antigen and a secretory signal.
2. The polyribonucleotide of item 1, wherein the polypeptide comprises an amino acid sequence according to SEQ ID NO: 1.
3. The polyribonucleotide of item 1, wherein the polypeptide comprises an amino acid sequence according to SEQ ID NO: 260.
4. The polyribonucleotide of any one of items 1-3, wherein the polyribonucleotide comprises a ribonucleic acid sequence according to SEQ ID NO: 16.
5. The polyribonucleotide of any one of items 1-3, wherein the polyribonucleotide comprises a ribonucleic acid sequence according to SEQ ID NO: 17.
6. The polyribonucleotide of any one of item 1-3, wherein the polyribonucleotide comprises a ribonucleic acid sequence according to SEQ ID NO: 18.
7. The polyribonucleotide of any one of item 1-3, wherein the polyribonucleotide comprises a ribonucleic acid sequence according to SEQ ID NO: 19.
8. The polyribonucleotide of any one of items 1-3, wherein the polyribonucleotide comprises a ribonucleic acid sequence according to SEQ ID NO: 147.
9. The polyribonucleotide of any one of items 1-3, wherein the polyribonucleotide comprises a ribonucleic acid sequence according to SEQ ID NO: 274.
10. The polyribonucleotide of any one of items 1-3, wherein the polyribonucleotide comprises a ribonucleic acid sequence according to SEQ ID NO: 275.
11. The polyribonucleotide of any one of items 1-3, wherein the polyribonucleotide comprises a ribonucleic acid sequence according to SEQ ID NO: 276.
12. The polyribonucleotide of any one of items 1-3, wherein the polyribonucleotide comprises a ribonucleic acid sequence according to SEQ ID NO: 277.
13. The polyribonucleotide of any one of items 1-3, wherein the polyribonucleotide comprises a ribonucleic acid sequence according to SEQ ID NO: 278.
14. The polyribonucleotide of any one of items 1-3, wherein the polyribonucleotide comprises a ribonucleic acid sequence according to SEQ ID NO: 279.
15. The polyribonucleotide of any one of items 1-3, wherein the polyribonucleotide comprises a ribonucleic acid sequence according to SEQ ID NO: 280.
16. The polyribonucleotide of any one of items 1-3, wherein the polyribonucleotide comprises a ribonucleic acid sequence according to SEQ ID NO: 281.
17. The polyribonucleotide of any one of items 1-3, wherein the polyribonucleotide comprises a ribonucleic acid sequence according to SEQ ID NO: 290.
18. The polyribonucleotide of any one of items 1-3, wherein the polyribonucleotide comprises a ribonucleic acid sequence according to SEQ ID NO: 291.
19. The polyribonucleotide of any one of items 1-3, wherein the polyribonucleotide comprises a ribonucleic acid sequence having at least 80% sequence identity to a ribonucleic acid sequence according to SEQ ID NO: 336.
20. The polyribonucleotide of any one of items 1-3, wherein the polyribonucleotide comprises a ribonucleic acid sequence having at least 90% sequence identity to a ribonucleic acid sequence according to SEQ ID NO: 336.
21. The polyribonucleotide of any one of items 1-3, wherein the polyribonucleotide comprises a ribonucleic acid sequence having at least 95% sequence identity to a ribonucleic acid sequence according to SEQ ID NO: 336.
22. The polyribonucleotide of any one of items 1-3, wherein the polyribonucleotide comprises a ribonucleic acid sequence according to SEQ ID NO: 336.
23. The polyribonucleotide of any one of items 1-22, wherein the secretory signal comprises or consists of an HSV secretory signal.
24. The polyribonucleotide of item 23, wherein the HSV secretory signal comprises or consists of an HSV2 secretory signal.
25. The polyribonucleotide of item 24, wherein the HSV2 secretory signal comprises or consists of an HSV2 glycoprotein D (gD) secretory signal.
26. The polyribonucleotide of item 25, wherein the HSV2 gD secretory signal comprises an amino acid sequence according to SEQ ID NO: 29.
27. The polyribonucleotide of item 25, wherein the HSV2 gD secretory signal comprises an amino acid sequence according to SEQ ID NO: 30.
28. The polyribonucleotide of item 25, wherein the HSV2 gD secretory signal comprises an amino acid sequence according to SEQ ID NO: 31.
29. The polyribonucleotide of any one of items 1-26, wherein the polyribonucleotide comprises a ribonucleic acid sequence according to SEQ ID NO: 54.
30. The polyribonucleotide of any one of items 1-26, wherein the polyribonucleotide comprises a ribonucleic acid sequence according to SEQ ID NO: 55.
31. The polyribonucleotide of any one of items 1-26, wherein the polyribonucleotide comprises a ribonucleic acid sequence according to SEQ ID NO: 56.
32. The polyribonucleotide of any one of items 1-26, wherein the polyribonucleotide comprises a ribonucleic acid sequence according to SEQ ID NO: 57.
33. The polyribonucleotide of any one of items 1-25 and 27, wherein the polyribonucleotide comprises a ribonucleic acid sequence according to SEQ ID NO: 58.
34. The polyribonucleotide of any one of items 1-25 and 27, wherein the polyribonucleotide comprises a ribonucleic acid sequence according to SEQ ID NO: 59.
35. The polyribonucleotide of any one of items 1-25 and 28, wherein the polyribonucleotide comprises a ribonucleic acid sequence according to SEQ ID NO: 62.
36. The polyribonucleotide of any one of items 1-25 and 28, wherein the polyribonucleotide comprises a ribonucleic acid sequence according to SEQ ID NO: 63.
37. The polyribonucleotide of any one of items 1-25 and 28, wherein the polyribonucleotide comprises a ribonucleic acid sequence according to SEQ ID NO: 301.
38. The polyribonucleotide of any one of items 1-25 and 28, wherein the polyribonucleotide comprises a ribonucleic acid sequence according to SEQ ID NO: 64.
39. The polyribonucleotide of any one of items 1-25, wherein the polypeptide comprises an amino acid sequence according to SEQ ID NO: 66.
40. The polyribonucleotide of any one of items 1-25, wherein the polyribonucleotide comprises a ribonucleic acid sequence according to SEQ ID NO: 108.
41. The polyribonucleotide of any one of items 1-25, wherein the polyribonucleotide comprises a ribonucleic acid sequence according to SEQ ID NO: 112.
42. The polyribonucleotide of any one of items 1-25, wherein the polyribonucleotide comprises a ribonucleic acid sequence according to SEQ ID NO: 113.
43. The polyribonucleotide of any one of items 1-25, wherein the polyribonucleotide comprises a ribonucleic acid sequence according to SEQ ID NO: 114.
44. The polyribonucleotide of item 23, wherein the HSV secretory signal comprises or consists of an HSV1 secretory signal.
45. The polyribonucleotide of item 44, wherein the HSV1 secretory signal comprises or consists of an HSV1 glycoprotein D (gD) secretory signal.
46. The polyribonucleotide of item 45, wherein the HSV1 gD secretory signal comprises an amino acid sequence according to SEQ ID NO: 213.
47. The polyribonucleotide of any one of items 1-23 and 44-45, wherein the polyribonucleotide comprises a ribonucleic acid sequence according to SEQ ID NO: 239.
48. The polyribonucleotide of any one of items 1-23 and 44-45, wherein the polyribonucleotide comprises a ribonucleic acid sequence according to SEQ ID NO: 240.
49. The polyribonucleotide of any one of items 1-23 and 44-45, wherein the polyribonucleotide comprises a ribonucleic acid sequence according to SEQ ID NO: 241.
50. The polyribonucleotide of any one of items 1-23 and 44-45, wherein the polyribonucleotide comprises a ribonucleic acid sequence according to SEQ ID NO: 242.
51. The polyribonucleotide of any one of items 1-23 and 44-45, wherein the polyribonucleotide comprises a ribonucleic acid sequence according to SEQ ID NO: 243.
52. The polyribonucleotide of any one of items 1-23 and 44-45, wherein the polyribonucleotide comprises a ribonucleic acid sequence according to SEQ ID NO: 189.
53. The polyribonucleotide of any one of items 1-23 and 44-45, wherein the polyribonucleotide comprises a ribonucleic acid sequence according to SEQ ID NO: 190.
54. The polyribonucleotide of any one of items 1-23 and 44-45, wherein the polyribonucleotide comprises a ribonucleic acid sequence according to SEQ ID NO: 348.
55. The polyribonucleotide of any one of items 1-23 and 44-45, wherein the polyribonucleotide comprises a ribonucleic acid sequence according to SEQ ID NO: 349.
56. The polyribonucleotide of any one of items 1-23 and 44-45, wherein the polyribonucleotide comprises a ribonucleic acid sequence according to SEQ ID NO: 191.
57. The polyribonucleotide of any one of items 1-23 and 44-45, wherein the polyribonucleotide comprises a ribonucleic acid sequence according to SEQ ID NO: 192.
58. The polyribonucleotide of item 24, wherein the HSV2 secretory signal comprises or consists of an HSV2 glycoprotein C (gC) secretory signal.
59. The polyribonucleotide of item 58, wherein the HSV2 gC secretory signal comprises an amino acid sequence according to SEQ ID NO: 32.
60. The polyribonucleotide of any one of items 1-24 and 59, wherein the polyribonucleotide comprises a ribonucleic acid sequence according to SEQ ID NO: 140.
61. The polyribonucleotide of any one of items 1-24 and 59, wherein the polyribonucleotide comprises a ribonucleic acid sequence according to SEQ ID NO: 302.
62. The polyribonucleotide of any one of items 1-24 and 59, wherein the polyribonucleotide comprises a ribonucleic acid sequence according to SEQ ID NO: 303.
63. The polyribonucleotide of any one of items 1-24 and 55, wherein the polyribonucleotide comprises a ribonucleic acid sequence according to SEQ ID NO: 304.
64. The polyribonucleotide of any one of items 1-24 and 54-59, wherein the polypeptide comprises an amino acid sequence according to SEQ ID NO: 131.
65. The polyribonucleotide of any one of items 1-24 and 54-59, wherein the polyribonucleotide comprises a ribonucleic acid sequence according to SEQ ID NO: 115.
66. The polyribonucleotide of item 24, wherein the HSV2 secretory signal comprises or consists of an HSV2 glycoprotein I (gI) secretory signal.
67. The polyribonucleotide of item 62, wherein the HSV2 gI secretory signal comprises an amino acid sequence according to SEQ ID NO: 215.
68. The polyribonucleotide of any one of items 1-24 and 67, wherein the polyribonucleotide comprises a ribonucleic acid sequence according to SEQ ID NO: 227.
69. The polyribonucleotide of any one of items 1-24 and 67, wherein the polyribonucleotide comprises a ribonucleic acid sequence according to SEQ ID NO: 228.
70. The polyribonucleotide of any one of items 1-24 and 67, wherein the polyribonucleotide comprises a ribonucleic acid sequence according to SEQ ID NO: 229.
71. The polyribonucleotide of any one of items 1-24 and 67, wherein the polyribonucleotide comprises a ribonucleic acid sequence according to SEQ ID NO: 230.
72. The polyribonucleotide of any one of items 1-24 and 67, wherein the polypeptide comprises an amino acid sequence according to SEQ ID NO: 161.
73. The polyribonucleotide of any one of items 1-24 and 67, wherein the polyribonucleotide comprises a ribonucleic acid sequence according to SEQ ID NO: 197.
74. The polyribonucleotide of any one of items 1-24 and 67, wherein the polyribonucleotide comprises a ribonucleic acid sequence according to SEQ ID NO: 198.
75. The polyribonucleotide of any one of items 1-24 and 67, wherein the polyribonucleotide comprises a ribonucleic acid sequence according to SEQ ID NO: 199.
76. The polyribonucleotide of any one of items 1-24 and 67, wherein the polyribonucleotide comprises a ribonucleic acid sequence according to SEQ ID NO: 200.
77. The polyribonucleotide of item 44, wherein the HSV1 secretory signal comprises or consists of an HSV1 glycoprotein B (gB) secretory signal.
78. The polyribonucleotide of item 77, wherein the HSV1 gB secretory signal comprises an amino acid sequence according to SEQ ID NO: 214.
79. The polyribonucleotide of any one of items 1-23, 44 and 77, wherein the polyribonucleotide comprises a ribonucleic acid sequence according to SEQ ID NO: 244.
80. The polyribonucleotide of any one of items 1-23, 44 and 77, wherein the polyribonucleotide comprises a ribonucleic acid sequence according to SEQ ID NO: 245.
81. The polyribonucleotide of any one of items 11-23, 44 and 77, wherein the polyribonucleotide comprises a ribonucleic acid sequence according to SEQ ID NO: 246.
82. The polyribonucleotide of any one of items 1-23, 44 and 77, wherein the polyribonucleotide comprises a ribonucleic acid sequence according to SEQ ID NO: 247.
83. The polyribonucleotide of any one of items 1-23, 44 and 77, wherein the polypeptide comprises an amino acid sequence according to SEQ ID NO: 160.
84. The polyribonucleotide of any one of items 1-23, 44 and 77, wherein the polyribonucleotide comprises a ribonucleic acid sequence according to SEQ ID NO: 193.
85. The polyribonucleotide of any one of items 1-23, 44 and 77, wherein the polyribonucleotide comprises a ribonucleic acid sequence according to SEQ ID NO: 194.
86. The polyribonucleotide of any one of items 1-23, 44 and 77, wherein the polyribonucleotide comprises a ribonucleic acid sequence according to SEQ ID NO: 350.
87. The polyribonucleotide of any one of items 1-23, 44 and 77, wherein the polyribonucleotide comprises a ribonucleic acid sequence according to SEQ ID NO: 195.
88. The polyribonucleotide of any one of items 1-23, 44 and 77, wherein the polyribonucleotide comprises a ribonucleic acid sequence according to SEQ ID NO: 196.
89. The polyribonucleotide of item 24, wherein the HSV2 secretory signal comprises or consists of an HSV2 glycoprotein E (gE) secretory signal.
90. The polyribonucleotide of any one of items 1-24 and 89, wherein the polypeptide comprises an amino acid sequence according to SEQ ID NO: 216.
91. The polyribonucleotide of any one of items 1-24 and 89, wherein the polyribonucleotide comprises a ribonucleic acid sequence according to SEQ ID NO: 252.
92. The polyribonucleotide of any one of items 1-24 and 89, wherein the polyribonucleotide comprises a ribonucleic acid sequence according to SEQ ID NO: 253.
93. The polyribonucleotide of any one of items 1-24 and 89, wherein the polyribonucleotide comprises a ribonucleic acid sequence according to SEQ ID NO: 254.
94. The polyribonucleotide of any one of items 1-24 and 89, wherein the polyribonucleotide comprises a ribonucleic acid sequence according to SEQ ID NO: 255.
95. The polyribonucleotide of any one of items 1-24 and 89, wherein the polypeptide comprises an amino acid sequence according to SEQ ID NO: 162.
96. The polyribonucleotide of any one of items 1-24 and 89, wherein the polyribonucleotide comprises a ribonucleic acid sequence according to SEQ ID NO: 201.
97. The polyribonucleotide of any one of items 1-24 and 89, wherein the polyribonucleotide comprises a ribonucleic acid sequence according to SEQ ID NO: 202.
98. The polyribonucleotide of any one of items 1-24 and 89, wherein the polyribonucleotide comprises a ribonucleic acid sequence according to SEQ ID NO: 345.
99. The polyribonucleotide of any one of items 1-24 and 89, wherein the polyribonucleotide comprises a ribonucleic acid sequence according to SEQ ID NO: 203.
100. The polyribonucleotide of any one of items 1-24 and 89, wherein the polyribonucleotide comprises a ribonucleic acid sequence according to SEQ ID NO: 204.
101. The polyribonucleotide of any one of items 1-22, wherein the secretory signal comprises or consists of an Ebola spike glycoprotein secretory signal.
102. The polyribonucleotide of item 101, wherein the Ebola spike glycoprotein secretory signal comprises an amino acid sequence according to SEQ ID NO: 217.
103. The polyribonucleotide of any one of items 1-22 and 101, wherein the polyribonucleotide comprises a ribonucleic acid sequence according to any one of SEQ ID NOs: 256-259.
104. The polyribonucleotide of any one of items 1-22 and 101, wherein the polyribonucleotide comprises a ribonucleic acid sequence according to SEQ ID NO: 205 or SEQ ID NO: 206.
105. The polyribonucleotide of any one of items 1-22 and 101, wherein the polypeptide comprises an amino acid sequence according to SEQ ID NO: 163.
106. The polyribonucleotide of any one of items 1-22 and 101, wherein the polyribonucleotide comprises a ribonucleic acid sequence according to SEQ ID NO: 207.
107. The polyribonucleotide of any one of items 1-22 and 101, wherein the polyribonucleotide comprises a ribonucleic acid sequence according to SEQ ID NO: 208.
108. A polyribonucleotide encoding a polypeptide, wherein the polypeptide comprises an HSV glycoprotein D (gD) antigen and a secretory signal.
109. The polyribonucleotide of item 108, wherein the polypeptide comprises an amino acid sequence according to SEQ ID NO: 2.
110. The polyribonucleotide of item 108 or 109, wherein the polyribonucleotide comprises a ribonucleic acid sequence according to SEQ ID NO: 20.
111. The polyribonucleotide of item 108 or 109, wherein the polyribonucleotide comprises a ribonucleic acid sequence according to SEQ ID NO: 21.
112. The polyribonucleotide of item 108 or 109, wherein the polyribonucleotide comprises a ribonucleic acid sequence according to SEQ ID NO: 22.
113. The polyribonucleotide of item 108 or 109, wherein the polyribonucleotide comprises a ribonucleic acid sequence according to SEQ ID NO: 143.
114. The polyribonucleotide of item 108 or 109, wherein the polyribonucleotide comprises a ribonucleic acid sequence according to SEQ ID NO: 286.
115. The polyribonucleotide of item 108 or 109, wherein the polyribonucleotide comprises a ribonucleic acid sequence according to SEQ ID NO: 340.
116. The polyribonucleotide of item 108 or 109, wherein the polyribonucleotide comprises a ribonucleic acid sequence according to SEQ ID NO: 23.
117. The polyribonucleotide of any one of items 108-116, wherein the secretory signal comprises or consists of an HSV secretory signal.
118. The polyribonucleotide of item 117, wherein the HSV secretory signal comprises or consists of an HSV2 secretory signal.
119. The polyribonucleotide of item 118, wherein the HSV2 secretory signal comprises or consists of an HSV2 glycoprotein D (gD) secretory signal.
120. The polyribonucleotide of item 119, wherein the HSV2 gD secretory signal comprises an amino acid sequence according to SEQ ID NO: 31.
121. The polyribonucleotide of any one of items 108-120, wherein the polyribonucleotide comprises a ribonucleic acid sequence according to SEQ ID NO: 61.
122. The polyribonucleotide of any one of items 108-120, wherein the polyribonucleotide comprises a ribonucleic acid sequence according to SEQ ID NO: 62.
123. The polyribonucleotide of any one of items 108-120, wherein the polyribonucleotide comprises a ribonucleic acid sequence according to SEQ ID NO: 63.
124. The polyribonucleotide of any one of items 108-120, wherein the polyribonucleotide comprises a ribonucleic acid sequence according to SEQ ID NO: 64.
125. The polyribonucleotide of any one of items 108-120, wherein the polypeptide comprises an amino acid sequence according to SEQ ID NO: 70.
126. The polyribonucleotide of any one of items 108-120, wherein the polyribonucleotide comprises a ribonucleic acid sequence according to SEQ ID NO: 116.
127. The polyribonucleotide of any one of items 108-120, wherein the polyribonucleotide comprises a ribonucleic acid sequence according to SEQ ID NO: 117.
128. The polyribonucleotide of any one of items 108-120, wherein the polyribonucleotide comprises a ribonucleic acid sequence according to SEQ ID NO: 118.
129. The polyribonucleotide of any one of items 108-120, wherein the polyribonucleotide comprises a ribonucleic acid sequence according to SEQ ID NO: 119.
130. The polyribonucleotide of any one of items 108-120, wherein the polyribonucleotide comprises a ribonucleic acid sequence according to SEQ ID NO: 346.
131. The polyribonucleotide of any one of items 108-120, wherein the polyribonucleotide comprises a ribonucleic acid sequence according to SEQ ID NO: 120.
132. A polyribonucleotide encoding a polypeptide, wherein the polypeptide comprises an HSV glycoprotein E (gE) antigen and a secretory signal.
133. The polyribonucleotide of item 132, wherein the polypeptide comprises an amino acid sequence according to SEQ ID NO: 3.
134. The polyribonucleotide of item 132 or 133, wherein the polyribonucleotide comprises a ribonucleic acid sequence according to SEQ ID NO: 24.
135. The polyribonucleotide of item 132 or 133, wherein the polyribonucleotide comprises a ribonucleic acid sequence according to SEQ ID NO: 25.
136. The polyribonucleotide of item 132 or 133, wherein the polyribonucleotide comprises a ribonucleic acid sequence according to SEQ ID NO: 26.
137. The polyribonucleotide of item 132 or 133, wherein the polyribonucleotide comprises a ribonucleic acid sequence according to SEQ ID NO: 149.
138. The polyribonucleotide of item 132 or 133, wherein the polyribonucleotide comprises a ribonucleic acid sequence according to SEQ ID NO: 27.
139. The polyribonucleotide of item 132 or 133, wherein the polyribonucleotide comprises a ribonucleic acid sequence according to SEQ ID NO: 282.
140. The polyribonucleotide of item 132 or 133, wherein the polyribonucleotide comprises a ribonucleic acid sequence according to SEQ ID NO: 283.
141. The polyribonucleotide of item 132 or 133, wherein the polyribonucleotide comprises a ribonucleic acid sequence according to SEQ ID NO: 284.
142. The polyribonucleotide of item 132 or 133, wherein the polyribonucleotide comprises a ribonucleic acid sequence according to SEQ ID NO: 285.
143. The polyribonucleotide of item 132 or 133, wherein the polyribonucleotide comprises a ribonucleic acid sequence according to SEQ ID NO: 341.
144. The polyribonucleotide of any one of items 132-143, wherein the secretory signal comprises or consists of an HSV secretory signal.
145. The polyribonucleotide of item 144, wherein the HSV secretory signal comprises or consists of an HSV2 secretory signal.
146. The polyribonucleotide of item 145, wherein the HSV2 secretory signal comprises or consists of an HSV2 glycoprotein D (gD) secretory signal.
147. The polyribonucleotide of item 146, wherein the HSV2 gD secretory signal comprises an amino acid sequence according to SEQ ID NO: 30.
148. The polyribonucleotide of any one of items 132-147, wherein the polyribonucleotide comprises a ribonucleic acid sequence according to SEQ ID NO: 58.
149. The polyribonucleotide of any one of items 132-147, wherein the polyribonucleotide comprises a ribonucleic acid sequence according to SEQ ID NO: 111.
150. The polyribonucleotide of any one of items 132-147, wherein the polyribonucleotide comprises a ribonucleic acid sequence according to SEQ ID NO: 59.
151. The polyribonucleotide of any one of items 132-147, wherein the polyribonucleotide comprises a ribonucleic acid sequence according to SEQ ID NO: 60.
152. The polyribonucleotide of any one of items 132-147, wherein the polypeptide comprises an amino acid sequence according to SEQ ID NO: 74.
153. The polyribonucleotide of any one of items 132-147, wherein the polyribonucleotide comprises a ribonucleic acid sequence according to SEQ ID NO: 125.
154. The polyribonucleotide of any one of items 132-147, wherein the polyribonucleotide comprises a ribonucleic acid sequence according to SEQ ID NO: 126.
155. The polyribonucleotide of any one of items 132-147, wherein the polyribonucleotide comprises a ribonucleic acid sequence according to SEQ ID NO: 127.
156. The polyribonucleotide of any one of items 132-147, wherein the polyribonucleotide comprises a ribonucleic acid sequence according to SEQ ID NO: 128.
157. The polyribonucleotide of any one of items 132-147, wherein the polyribonucleotide comprises a ribonucleic acid sequence according to SEQ ID NO: 347.
158. The polyribonucleotide of any one of items 132-147, wherein the polyribonucleotide comprises a ribonucleic acid sequence according to SEQ ID NO: 129.
159. The polyribonucleotide of item 145, wherein the HSV2 secretory signal comprises or consists of an HSV2 glycoprotein E (gE) secretory signal.
160. The polyribonucleotide of item 159, wherein the HSV2 gE secretory signal comprises an amino acid sequence according to SEQ ID NO: 33.
161. The polyribonucleotide of any one of items 132-145 and 160, wherein the polyribonucleotide comprises a ribonucleic acid sequence according to SEQ ID NO: 141.
162. The polyribonucleotide of any one of items 132-145 and 160, wherein the polyribonucleotide comprises a ribonucleic acid sequence according to SEQ ID NO: 305.
163. The polyribonucleotide of any one of items 132-145 and 160, wherein the polyribonucleotide comprises a ribonucleic acid sequence according to SEQ ID NO: 306.
164. The polyribonucleotide of any one of items 132-145 and 160, wherein the polyribonucleotide comprises a ribonucleic acid sequence according to SEQ ID NO: 307.
165. The polyribonucleotide of any one of items 132-145 and 160, wherein the HSV2 gE secretory signal comprises an amino acid sequence according to SEQ ID NO: 292.
166. The polyribonucleotide of any one of items 132-145 and 165, wherein the polyribonucleotide comprises a ribonucleic acid sequence according to SEQ ID NO: 308.
167. The polyribonucleotide of any one of items 132-145 and 159, wherein the polypeptide comprises an amino acid sequence according to SEQ ID NO: 132.
168. The polyribonucleotide of any one of items 132-145 and 159, wherein the polyribonucleotide comprises a ribonucleic acid sequence according to SEQ ID NO: 130.
169. The polyribonucleotide of item 144, wherein the HSV secretory signal comprises or consists of an HSV1 secretory signal.
170. The polyribonucleotide of item 169, wherein the HSV1 secretory signal comprises or consists of an HSV1 glycoprotein D (gD) secretory signal.
171. The polyribonucleotide of item 170, wherein the HSV1 gD secretory signal comprises an amino acid sequence according to SEQ ID NO: 213.
172. The polyribonucleotide of item 170, wherein the polyribonucleotide comprises a ribonucleic acid sequence according to SEQ ID NO: 239.
173. The polyribonucleotide of item 170, wherein the polyribonucleotide comprises a ribonucleic acid sequence according to SEQ ID NO: 240.
174. The polyribonucleotide of item 170, wherein the polyribonucleotide comprises a ribonucleic acid sequence according to SEQ ID NO: 241.
175. The polyribonucleotide of item 170, wherein the polyribonucleotide comprises a ribonucleic acid sequence according to SEQ ID NO: 242.
176. The polyribonucleotide of item 170, wherein the polyribonucleotide comprises a ribonucleic acid sequence according to SEQ ID NO: 243.
177. The polyribonucleotide of any one of items 132-145 and 159, wherein the polypeptide comprises an amino acid sequence according to SEQ ID NO: 164.
178. The polyribonucleotide of any one of items 132-145 and 159, wherein the polyribonucleotide comprises a ribonucleic acid sequence according to SEQ ID NO: 209.
179. The polyribonucleotide of any one of items 132-145 and 159, wherein the polyribonucleotide comprises a ribonucleic acid sequence according to SEQ ID NO: 210.
180. The polyribonucleotide of any one of items 132-145 and 159, wherein the polyribonucleotide comprises a ribonucleic acid sequence according to SEQ ID NO: 211.
181. The polyribonucleotide of any one of items 132-145 and 159, wherein the polyribonucleotide comprises a ribonucleic acid sequence according to SEQ ID NO: 212.
182. The polyribonucleotide of any one of items 1-181, wherein the secretory signal is located at the N-terminus of the polypeptide.
183. The polyribonucleotide of any one of items 1-182, wherein the polyribonucleotide is an isolated polyribonucleotide.
184. The polyribonucleotide of any one of items 1-183, wherein the polyribonucleotide is an engineered polyribonucleotide.
185. The polyribonucleotide of any one of items 1-183, wherein the polyribonucleotide is a codon-optimized polyribonucleotide.
186. An RNA construct comprising in 5′ to 3′ order:
   (i) a 5′ UTR;
   (ii) a polyribonucleotide of any one of items 1-185;
   (iii) a 3′ UTR; and
   (iv) a polyA tail sequence.
187. The RNA construct of item 186, wherein
   (i) the 5′ UTR comprises or consists of a modified human alpha-globin 5′-UTR; and
   (ii) the 3′ UTR that comprises or consists of a first sequence from the amino terminal enhancer of split (AES) messenger RNA and a second sequence from the mitochondrial encoded 12S ribosomal RNA.

188. The RNA construct of item 186 or 187, wherein the 5′ UTR consists of a ribonucleic acid sequence according to SEQ ID NO: 152.

189. The RNA construct of any one of items 186-188, wherein the 3′ UTR consists of a ribonucleic acid sequence according to SEQ ID NOs: 158.
190. The RNA construct of any one of items 186-189, wherein the polyA tail sequence is a split polyA tail sequence.
191. The RNA construct of item 190, wherein the split polyA tail sequence consists of a ribonucleic acid sequence according to SEQ ID NO: 155.
192. The RNA construct of any one of items 186-191, further comprising a 5′ cap.
193. The RNA construct of item 192, further comprising a cap proximal sequence comprising positions +1, +2, +3, +4, and +5 of the polyribonucleotide.
194. The RNA construct of item 192 or 193, wherein the 5′ cap comprises or consists of m7(3′OMeG)(5′)ppp(5′)(2′OMeA₁)pG₂, wherein A₁ is position +1 of the polyribonucleotide, and G₂ is position +2 of the polyribonucleotide.
195. The RNA construct of item 193 or 194, wherein the cap proximal sequence comprises A₁ and G₂ of the Cap1 structure, and a sequence comprising: A₃A₄U₅ (SEQ ID NO: 150) at positions +3, +4 and +5 respectively of the polyribonucleotide.
196. The RNA construct of any one of items 186-195, wherein the polyribonucleotide includes modified uridines in place of all uridines, optionally wherein modified uridines are each N1-methyl-pseudouridine.
197. A composition comprising one or more polyribonucleotides of any one of items 1-185.
198. A composition comprising one or more RNA constructs of any one of 186-196.
199. The composition of item 197 or 198, wherein the composition further comprises lipid nanoparticles, polyplexes (PLX), lipidated polyplexes (LPLX), or liposomes, wherein the one or more polyribonucleotides are fully or partially encapsulated within the lipid nanoparticles, polyplexes (PLX), lipidated polyplexes (LPLX), or liposomes.
200. The composition of any one of items 197-199, wherein the composition further comprises lipid nanoparticles, wherein the one or more polyribonucleotides are encapsulated within the lipid nanoparticles.
201. The composition of item 200, wherein the lipid nanoparticle comprises
   (a) a polymer-conjugated lipid;
   (b) a cationic lipid; and
   (c) one or more neutral lipids.
202. The composition of item 201, wherein the polymer-conjugated lipid comprises a PEG-conjugated lipid.
203. The composition of item 201 or 202, wherein the polymer-conjugated lipid comprises 2-[(polyethylene glycol)-2000]-N,N-ditetradecylacetamide.
204. The composition of any one of items 201 to 203, wherein the one or more neutral lipids comprise (2R)-2,3-Bis(octadecanoyloxy) propyl 2-(trimethylazaniumyl)ethyl phosphate.
205. The composition of any one of items 201 to 204, wherein the one or more neutral lipids comprise cholesterol.
206. The composition of any one of items 201 to 205, wherein the cationic lipid comprises [(4-Hydroxybutyl)azanediyl]di(hexane-6,1-diyl)bis(2-hexyldecanoate).
207. The composition of any one of items 201 to 206, wherein the lipid nanoparticle comprises:
   (a) 2-[(polyethylene glycol)-2000]-N,N-ditetradecylacetamide;
   (b) (2R)-2,3-Bis(octadecanoyloxy) propyl 2-(trimethylazaniumyl)ethyl phosphate;
   (c) cholesterol; and
   (d) [(4-Hydroxybutyl)azanediyl]di(hexane-6,1-diyl)bis(2-hexyldecanoate).
208. The composition of any one of items 201 to 207, wherein the lipid nanoparticle comprises:
   (a) the polymer-conjugated lipid at about 1-2.5 mol % of the total lipids;
   (b) the cationic lipid at 35-65 mol % of the total lipids; and
   (c) the one or more neutral lipids are present in 35-65 mol % of the total lipids.
209. The composition of any one of items 1 to 208, wherein the therapeutically effective amount of the one or more polyribonucleotides is 0.5 μg to 250 μg.
210. The composition of any one of items 1 to 209, wherein the therapeutically effective amount of the one or more polyribonucleotides is 1 μg to 200 μg.
211. The composition of any one of items 1 to 210, wherein the therapeutically effective amount of the one or more polyribonucleotides is 2 μg to 100 μg.
212. The composition of any one of items 1 to 211, wherein the therapeutically effective amount of the one or more polyribonucleotides is 3 μg to 60 μg.
213. The composition of any one of items 1 to 212, wherein the therapeutically effective amount of the one or more polyribonucleotides is about 3 μg.
214. The composition of any one of items 1 to 213, wherein the therapeutically effective amount of the one or more polyribonucleotides is about 10 μg.
215. The composition of any one of items 1 to 214 wherein the therapeutically effective 215 of the one or more polyribonucleotides is about 30 μg.
216. The composition of any one of items 1 to 215, wherein the therapeutically effective amount of the one or more polyribonucleotides is about 60 μg.
217. A pharmaceutical composition comprising the composition of any one of items 197-200 and at least one pharmaceutically acceptable excipient.
218. The pharmaceutical composition of item 217, wherein the pharmaceutical comprises a cryoprotectant, optionally wherein the cryoprotectant is sucrose.
219. The pharmaceutical composition of item 217 or 218, wherein the pharmaceutical comprises an aqueous buffered solution, optionally wherein the aqueous buffered solution comprises one or more of Tris base, Tris HCl, NaCl, KCl, Na₂HPO₄, and KH₂PO₄.
220. A combination comprising:
   a first gC polyribonucleotide according to any one of items 1-107; and
   a second gD polyribonucleotide according to any one of items 108-131.
221. A combination comprising:
   a first pharmaceutical composition comprising a first gC polyribonucleotide, wherein the first gC polyribonucleotide is a polyribonucleotide according to any one of items 1-107; and
   a second pharmaceutical composition comprising a second gD polyribonucleotide, wherein the second gD polyribonucleotide is a polyribonucleotide according to any one of items 108-131.
222. The combination of item 220 or 221, wherein the first gC polyribonucleotide is a polyribonucleotide according to item 57.
223. The combination of item 220 or 221, wherein the first gC polyribonucleotide is a polyribonucleotide according to item 54.
224. The combination of item 220 or 221, wherein the first gC polyribonucleotide is a polyribonucleotide according to item 86.
225. The combination of item 220 or 221, wherein the first gC polyribonucleotide is a polyribonucleotide according to item 98.
226. The combination of item 220 or 221, wherein the first gC polyribonucleotide is a polyribonucleotide according to item 55.
227. The combination of any one of items 220 to 226, wherein the second gD polyribonucleotide is a gD polyribonucleotide according to item 112.
228. The combination of any one of items 220 to 226, wherein the second gD polyribonucleotide is a gD polyribonucleotide according to item 129.
229. The combination of any one of items 220 to 226, wherein the second gD polyribonucleotide is a gD polyribonucleotide according to item 130.
230. A combination comprising:
   a first gC polyribonucleotide according to any one of items 1-107; and
   a second gE polyribonucleotide according to any one of items 132-185.
231. A combination comprising:
   a first gC pharmaceutical composition comprising a first gC polyribonucleotide, wherein the first gC polyribonucleotide is a polyribonucleotide according to any one of items 1-107; and
   a second gE pharmaceutical composition comprising a second gE polyribonucleotide,
   wherein the second gE polyribonucleotide is a polyribonucleotide according to any one of items 132-185.
232. The combination of item 230 or 231, wherein the first gC polyribonucleotide is a polyribonucleotide according to item 57.
233. The combination of item 230 or 231, wherein the first gC polyribonucleotide is a polyribonucleotide according to item 54.
234. The combination of item 230 or 231, wherein the first gC polyribonucleotide is a polyribonucleotide according to item 86.
235. The combination of item 230 or 231, wherein the first gC polyribonucleotide is a polyribonucleotide according to item 98.
236. The combination of item 230 or 231, wherein the first gC polyribonucleotide is a polyribonucleotide according to item 55.
237. The combination of any one of items 230 to 236, wherein the second gE polyribonucleotide is a polyribonucleotide according to item 137.
238. The combination of any one of items 230 to 236, wherein the second gE polyribonucleotide is a polyribonucleotide according to item 137.
239. A combination comprising:
   a first gD polyribonucleotide according to any one of items 108-131; and
   a second gE polyribonucleotide according to any one of items 132-185.
240. A combination comprising:
   a first pharmaceutical composition comprising a first gD polyribonucleotide, wherein the first gD polyribonucleotide is a polyribonucleotide according to any one of items 108-131; and
   a second pharmaceutical composition comprising a second gE polyribonucleotide, wherein the second gE polyribonucleotide is a polyribonucleotide according to any one of items 132-185.
241. The combination of item 239 or 240, wherein the first gD polyribonucleotide is a polyribonucleotide according to item 112.
242. The combination of items 239 or 240, wherein the second gD polyribonucleotide is a polyribonucleotide according to item 129.
243. The combination of items 239 or 240, wherein the second gD polyribonucleotide is a polyribonucleotide according to item 130.
244. The combination of any one of items 239 to 243, wherein the second gE polyribonucleotide is a polyribonucleotide according to item 137.
245. The combination of any one of items 239 to 243, wherein the second gE polyribonucleotide is a polyribonucleotide according to item 157.
246. A combination comprising:
   a first gC polyribonucleotide according to any one of items 1-107;
   a second gD polyribonucleotide according to any one of items 108-131; and
   a third gE polyribonucleotide according to any one of items 132-185.
247. A combination comprising:
   a first pharmaceutical composition comprising a first gC polyribonucleotide, wherein the first gC polyribonucleotide is a polyribonucleotide according to any one of items 1-107;
   a second pharmaceutical composition comprising a second gD polyribonucleotide,
   wherein the second gD polyribonucleotide is a polyribonucleotide according to any one of items 108-131; and
   a third gE pharmaceutical composition comprising a third gE polyribonucleotide, wherein the third polyribonucleotide is a polyribonucleotide according to any one of items 132-185.
248. The combination of item 246 or 247, wherein the first polyribonucleotide is a polyribonucleotide according to item 55.
249. The combination of any one of items 246 to 248, wherein the second polyribonucleotide is a polyribonucleotide according to item 130.
250. The combination of any one of items 246 to 249, wherein the second polyribonucleotide is a polyribonucleotide according to item 157.
251. A method comprising administering a polyribonucleotide according to any one of items 1-185 to a subject.
252. A method comprising administering an RNA construct according to any one of items 186-196 to a subject.
253. A method comprising administering a composition according to any one of items 197-200 to a subject.
254. A method comprising administering one or more doses of the pharmaceutical composition of any one of items 217 to 219 to a subject.
255. The pharmaceutical composition of any one of items 217 to 219 for use in the treatment of an HSV infection comprising administering one or more doses of the pharmaceutical composition to a subject.
256. The pharmaceutical composition of any one of items 217 to 219 for use in the prevention of an HSV infection comprising administering one or more doses of the pharmaceutical composition to a subject.
257. The method of item 254 or the pharmaceutical composition for use of item 255 or 256, comprising administering two or more doses of the pharmaceutical composition to a subject.
258. The method of item 254 or 257, or the pharmaceutical composition for use of any one of item 255 or 256, comprising administering three or more doses of the pharmaceutical composition to a subject.
259. A method comprising administering a combination of any one of items 220-250 to a subject.
260. The method of item 259, wherein the first pharmaceutical composition and the second pharmaceutical composition are administered on the same day.
261. The method of item 259 or 260, wherein the first pharmaceutical composition and the second pharmaceutical composition are administered on different days.
262. The method of any one of items 259 to 261, wherein the first pharmaceutical composition and the second pharmaceutical composition are administered to the subject at different locations on the subject's body.
263. The method of any one of items 251 to 254 and 258 to 262, wherein the method is a method of treating an HSV infection.
264. The method of any one of items 251 to 254 and 257 to 263, wherein the method is a method of preventing an HSV infection.
265. The method of any one of items 251 to 254 and 257 to 264, wherein the subject has or is at risk of developing an HSV infection.
266. The method of any one of items 251 to 254 and 257 to 265, wherein the subject is a human.
267. The method of any one of items 251 to 254 and 257 to 266, wherein administration induces an anti-HSV immune response in the subject.
268. The method of item 267, wherein the anti-HSV immune response in the subject comprises an adaptive immune response.
269. The method of item 267 or 268, wherein the anti-HSV immune response in the subject comprises a T-cell response.
270. The method of item 269, wherein the T-cell response is or comprises a CD4+ T cell response.
271. The method of item 269 or 270, wherein the T-cell response is or comprises a CD8+ T cell response.
272. The method of any one of items 269 to 271, wherein the anti-HSV immune system response comprises a B-cell response.
273. The method of any one of items 269 to 272, wherein the anti-HSV immune system response comprises the production of antibodies directed against the one or more HSV antigens.
274. Use of the pharmaceutical composition of any one of items 217 to 219 in the treatment of an HSV infection.
275. Use of the pharmaceutical composition of any one of items 217 to 219 in the prevention of an HSV infection.
276. Use of the pharmaceutical composition of any one of items 217 to 219 in inducing an anti-HSV immune response in a subject.
277. A polypeptide encoded by a polyribonucleotide of any one of items 1-185.
278. A polypeptide encoded by an RNA construct of any one of items 186-196.
279. A host cell comprising a polyribonucleotide of any one of items 1-185.
280. A host cell comprising an RNA construct of any one of items 186-196.
281. A host cell comprising a polypeptide of claim 279 or 280.

### EQUIVALENTS

It is to be understood that while the disclosure has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. 1. A combination comprising:
a polyribonucleotide encoding an HSV glycoprotein C (gC) antigen or antigenic fragment thereof, a polyribonucleotide encoding an HSV glycoprotein D (gD) antigen or antigenic fragment thereof, and a polyribonucleotide encoding an HSV glycoprotein E (gE) antigen or antigenic fragment thereof, wherein:
(i) the polyribonucleotide encoding HSV gC antigen or antigenic fragment thereof comprises a ribonucleic acid sequence with at least 90% sequence identity to SEQ ID NO: 336;
(ii) the polyribonucleotide encoding HSV gD antigen or antigenic fragment thereof comprises a ribonucleic acid sequence with at least 90% sequence identity to SEQ ID NO: 340;
(iii) the polyribonucleotide encoding HSV gE antigen or antigenic fragment thereof comprises a ribonucleic acid sequence with at least 90% sequence identity to SEQ ID NO: 283; or
(iv) any combination thereof.

2. The combination of claim 1, wherein the polyribonucleotide encoding an HSV gC antigen or antigenic fragment thereof further encodes a secretory signal.

3. The combination of claim 2, wherein the secretory signal comprises an HSV secretory signal.

4. The combination of claim 3, wherein the HSV secretory signal is a HSV glycoprotein D (gD) secretory signal.

5. The combination of claim 4, wherein the HSV gD secretory signal is a HSV1 gD secretory signal.

6. The combination of claim 5, wherein the HSV1 gD secretory signal comprises an amino acid sequence according to SEQ ID NO: 213.

7. The combination of claim 6, wherein the polyribonucleotide encoding an HSV gC antigen or antigenic fragment thereof comprises a nucleic acid sequence with at least 90% sequence identity to SEQ ID NO: 240.

8. The combination of any one of claims 1 to 7, wherein the polyribonucleotide encoding an HSV gD antigen or antigenic fragment thereof further encodes a secretory signal.

9. The combination of claim 8, wherein the secretory signal comprises an HSV secretory signal.

10. The combination of claim 9, wherein the HSV secretory signal comprises an HSV gD secretory signal.

11. The combination of claim 10, wherein the HSV gD secretory signal comprises an HSV2 gD secretory signal.

12. The combination of claim 11, wherein the HSV2 gD secretory signal comprises an amino acid sequence according to SEQ ID NO: 29.

13. The combination of claim 12, wherein the polyribonucleotide encoding an HSV gD antigen or antigenic fragment thereof comprises a nucleic acid sequence with at least 90% sequence identity to SEQ ID NO: 56.

14. The combination of any one of claims 1 to 13, wherein the polyribonucleotide encoding an HSV gD antigen or antigenic fragment thereof further encodes a secretory signal.

15. The combination of claim 14, wherein the secretory signal comprises an HSV secretory signal.

16. The combination of claim 15, wherein the HSV secretory signal comprises an HSV gD secretory signal.

17. The combination of claim 16, wherein the HSV gD secretory signal comprises an HSV2 gD secretory signal.

18. The combination of claim 17, wherein the HSV2 gD secretory signal comprises an amino acid sequence according to SEQ ID NO: 30.

19. The combination of claim 18, wherein the polyribonucleotide encoding an HSV gD antigen or antigenic fragment thereof comprises a nucleic acid sequence with at least 90% sequence identity to SEQ ID NO: 59.

20. The combination of any one of claims 1 to 19, wherein the polyribonucleotide encoding HSV gC antigen or antigenic fragment thereof comprises a nucleic acid sequence with at least 90% sequence identity to SEQ ID NO: 349.

21. The combination of any one of claims 1 to 20, wherein the polyribonucleotide encoding HSV gD antigen or antigenic fragment thereof comprises a nucleic acid sequence with at least 90% sequence identity to SEQ ID NO: 352.

22. The combination of any one of claims 1 to 21, wherein the polyribonucleotide encoding HSV gE antigen or antigenic fragment thereof comprises a nucleic acid sequence with at least 90% sequence identity to SEQ ID NO: 353.

23. The combination of any one of claims 1 to 22, wherein at least one of the polyribonucleotides comprises modified uridines.

24. The combination of any one of claims 1 to 23, wherein the modified uridines are N1-methyl-pseudouridine.

25. The combination of any one of claims 1 to 24, wherein one or more of the polyribonucleotides comprises in 5′ to 3′ order:
(i) a 5′ UTR;
(ii) a polyribonucleotide of any one of claims 1-24;
(iii) a 3′ UTR; and
(iv) a polyA tail sequence.

26. The combination of claim 25, wherein
(i) the 5′ UTR comprises or consists of a modified human alpha-globin 5′-UTR; and
(ii) the 3′ UTR that comprises or consists of a first sequence from the amino terminal enhancer of split (AES) messenger RNA and a second sequence from the mitochondrial encoded 12S ribosomal RNA.

27. The combination of claim 25 or 26, wherein the 5′ UTR consists of a ribonucleic acid sequence according to SEQ ID NO: 152.

28. The combination of any one of claims 25 to 27, wherein the 3′ UTR consists of a ribonucleic acid sequence according to SEQ ID NOs: 158.

29. The combination of any one of claims 25 to 28, wherein the polyA tail sequence is a split polyA tail sequence.

30. The combination of claim 29, wherein the split polyA tail sequence consists of a ribonucleic acid sequence according to SEQ ID NO: 155.

31. The combination of any one of claims 25 to 30, further comprising a 5′ cap.

32. The combination of claim 31, further comprising a cap proximal sequence comprising positions +1, +2, +3, +4, and +5 of the polyribonucleotide.

33. The combination of claims 31 and 32, wherein the 5′ cap comprises or consists of m7(3′OMeG)(5′)ppp(5′)(2′OMeA₁)pG₂, wherein A₁ is position +1 of the polyribonucleotide, and G₂ is position +2 of the polyribonucleotide.

34. The combination of claim 32 or 33, wherein the cap proximal sequence comprises A₁ and G₂ of the Cap1 structure, and a sequence comprising: A₃A₄U₅ (SEQ ID NO: 150) at positions +3, +4 and +5 respectively of the polyribonucleotide.

35. The combination of any one of claims 1 to 34, wherein the composition further comprises one or more HSV glycoproteins.

36. The combination of claim 35, wherein the one or more HSV glycoproteins comprise an HSV glycoprotein B (gB) antigen or antigenic fragment thereof, an HSV glycoprotein E (gE) antigen or antigenic fragment thereof, an HSV glycoprotein G (gG) antigen or antigenic fragment thereof, an HSV glycoprotein H (gH) antigen or antigenic fragment thereof, an HSV glycoprotein I (g) antigen or antigenic fragment thereof, an HSV glycoprotein L (gL) antigen or antigenic fragment thereof, or a combination thereof.

37. The combination of any one of claims 1 to 36, wherein one or more of the polyribonucleotides are fully or partially encapsulated within lipid nanoparticles, polyplexes (PLX), lipidated polyplexes (LPLX), or liposomes.

38. The combination of any one of claims 1 to 37, wherein the polyribonucleotide sequences are in a single composition.

39. The combination of any one of claims 1 to 38 for use in the prevention of an HSV infection comprising administering one or more doses of the pharmaceutical composition to a subject.

40. The combination of any one of claims 1 to 38 for use in the treatment of an HSV infection comprising administering one or more doses of the pharmaceutical composition to a subject.

41. A composition comprising a combination according to any one of claims 1 to 38.

42. A pharmaceutical composition comprising a combination according to any one of claims 1 to 38.

43. A method comprising administering a combination according to any one of claims 1 to 38 to a subject.

44. A method comprising administering a composition according to claim 41 to a subject.

45. A method comprising administering one or more doses of the pharmaceutical composition of claim 42 to a subject.

46. The method of any one of claims 43 to 45, wherein the method is a method of treating an HSV infection.

47. The method of any one of claims 43 to 45, wherein the method is a method of preventing an HSV infection.

48. Use of the combination of any one of claims 1 to 38 in the prevention of an HSV infection.

49. Use of the composition of claim 41 in the prevention of an HSV infection.

50. Use of the pharmaceutical composition of claim 42 in the prevention of an HSV infection.

51. Use of the combination of any one of claims 1 to 38 in the treatment of an HSV infection.

52. Use of the composition of claim 41 in the treatment of an HSV infection.

53. Use of the pharmaceutical composition of claim 42 in the treatment of an HSV infection.

54. A combination comprising:
a polyribonucleotide encoding a polypeptide comprising
(i) an amino acid sequence with at least 90% identity to SEQ ID NO: 213 and
(ii) an amino acid sequence with at least 90% identity to SEQ ID NO: 260;
a polyribonucleotide encoding a polypeptide comprising
(i) an amino acid sequence with at least 90% identity to SEQ ID NO: 29 and
(ii) an amino acid sequence with at least 90% identity to SEQ ID NO: 2; and
a polyribonucleotide encoding a polypeptide comprising an
(i) an amino acid sequence with at least 90% identity to SEQ ID NO: 30 and
(ii) an amino acid sequence with at least 90% identity to SEQ ID NO: 3.

55. The combination of claim 54, wherein
a polyribonucleotide encoding a polypeptide comprising an amino acid sequence with at least 90% identity to SEQ ID NO: 159;
a polyribonucleotide encoding a polypeptide comprising an amino acid sequence with at least 90% identity to SEQ ID NO: 70; and
a polyribonucleotide encoding a polypeptide comprising an amino acid sequence with at least 90% identity to SEQ ID NO: 75.


SEQUENCE LISTING (383 sequences)

SEQ ID NO: 1 - protein, 400 aa, Human alphaherpesvirus 2
ASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 2 - protein, 306 aa, Human alphaherpesvirus 2
KYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHH

SEQ ID NO: 3 - protein, 382 aa, Human alphaherpesvirus 2
RTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRA

SEQ ID NO: 4 - DNA, 1203 bp, Human alphaherpesvirus 2
  source 1..1203: mol_type = genomic DNA
gcctcccccggccgcaccatcaccgtgggcccccgcggcaacgcctccaacgccgccccctccgcctccccccgcaacgcctccgccccccgcaccacccccacccccccccagccccgcaaggccaccaagtccaaggcctccaccgccaagcccgcccccccccccaagaccggcccccccaagacctcctccgagcccgtgcgctgcaaccgccacgaccccctggcccgctacggctcccgcgtgcagatccgctgccgcttccccaactccacccgcaccgagttccgcctgcagatctggcgctacgccaccgccaccgacgccgagatcggcaccgccccctccctggaggaggtgatggtgaacgtgtccgccccccccggcggccagctggtgtacgactccgcccccaaccgcaccgacccccacgtgatctgggccgagggcgccggccccggcgcctccccccgcctgtactccgtggtgggccccctgggccgccagcgcctgatcatcgaggagctgaccctggagacccagggcatgtactactgggtgtggggccgcaccgaccgcccctccgcctacggcacctgggtgcgcgtgcgcgtgttccgccccccctccctgaccatccacccccacgccgtgctggagggccagcccttcaaggccacctgcaccgccgccacctactaccccggcaaccgcgccgagttcgtgtggttcgaggacggccgccgcgtgttcgaccccgcccagatccacacccagacccaggagaaccccgacggcttctccaccgtgtccaccgtgacctccgccgccgtgggcggccagggccccccccgcaccttcacctgccagctgacctggcaccgcgactccgtgtccttctcccgccgcaacgcctccggcaccgcctccgtgctgccccgccccaccatcaccatggagttcaccggcgaccacgccgtgtgcaccgccggctgcgtgcccgagggcgtgaccttcgcctggttcctgggcgacgactcctcccccgccgagaaggtggccgtggcctcccagacctcctgcggccgccccggcaccgccaccatccgctccaccctgcccgtgtcctacgagcagaccgagtacatctgccgcctggccggctaccccgacggcatccccgtgctggagcaccactaa

SEQ ID NO: 5 - DNA, 1206 bp, synthetic construct
  source 1..1206: mol_type = other DNA
agcgcttctcccggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggcccaggcgcaagccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactga

SEQ ID NO: 6 - DNA, 1203 bp, synthetic construct
  source 1..1203: mol_type = other DNA
gcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 7 - DNA, 1203 bp, synthetic construct
  source 1..1203: mol_type = other DNA
gcctcccccggcagaaccatcaccgtgggccccagaggcaacgcctccaacgccgccccctccgcctcccccagaaacgcctccgcccccagaaccacccccacccctccccagcccagaaaagccaccaaatccaaagcctccaccgccaaacccgcccctcctcccaaaaccggccctcccaaaacctcctccgaacccgtgagatgcaacagacacgatcccctggccagatacggctccagagtgcagatcagatgcagattccccaactccaccagaaccgaattcagactgcagatctggagatacgccaccgccaccgatgccgaaatcggcaccgccccctccctggaagaagtgatggtgaacgtgtccgcccctcctggcggccagctggtgtacgattccgcccccaacagaaccgatccccacgtgatctgggccgaaggcgccggccccggcgcctcccccagactgtactccgtggtgggccccctgggcagacagagactgatcatcgaagaactgaccctggaaacccagggcatgtactactgggtgtggggcagaaccgatagaccctccgcctacggcacctgggtgagagtgagagtgttcagacccccttccctgaccatccacccccacgccgtgctggaaggccagcccttcaaagccacctgcaccgccgccacctactaccccggcaacagagccgaattcgtgtggttcgaagatggcagaagggtgttcgatcccgcccagatccacacccagacccaggaaaaccccgacggcttctccaccgtgtccaccgtgacctccgccgccgtgggcggccagggccctcccagaaccttcacctgccagctgacctggcacagagactccgtgtccttctccagaagaaacgcctccggcaccgcctccgtgctgcccagacccaccatcaccatggaattcaccggcgatcacgccgtgtgcaccgccggctgcgtgcccgaaggcgtgaccttcgcctggttcctgggcgatgattcctcccccgccgaaaaagtggccgtggcctcccagacctcctgcggcagacccggcaccgccaccatcagatccaccctgcccgtgtcctacgaacagaccgaatacatctgcagactggccggctaccccgatggcatccccgtgctggaacaccactga

SEQ ID NO: 8 - DNA, 921 bp, Human alphaherpesvirus 2
  source 1..921: mol_type = genomic DNA
aagtacgccctggccgacccctccctgaagatggccgaccccaaccgcttccgcggcaagaacctgcccgtgctggaccagctgaccgacccccccggcgtgaagcgcgtgtaccacatccagccctccctggaggaccccttccagcccccctccatccccatcaccgtgtactacgccgtgctggagcgcgcctgccgctccgtgctgctgcacgccccctccgaggccccccagatcgtgcgcggcgcctccgacgaggcccgcaagcacacctacaacctgaccatcgcctggtaccgcatgggcgacaactgcgccatccccatcaccgtgatggagtacaccgagtgcccctacaacaagtccctgggcgtgtgccccatccgcacccagccccgctggtcctactacgactccttctccgccgtgtccgaggacaacctgggcttcctgatgcacgcccccgccttcgagaccgccggcacctacctgcgcctggtgaagatcaacgactggaccgagatcacccagttcatcctggagcaccgcgcccgcgcctcctgcaagtacgccctgcccctgcgcatcccccccgccgcctgcctgacctccaaggcctaccagcagggcgtgaccgtggactccatcggcatgctgccccgcttcatccccgagaaccagcgcaccgtggccctgtactccctgaagatcgccggctggcacggccccaagcccccctacacctccaccctgctgccccccgagctgtccgacaccaccaacgccacccagcccgagctggtgcccgaggaccccgaggactccgccctgctggaggaccccgccggcaccgtgtcctcccagatcccccccaactggcacatcccctccatccaggacgtggccccccaccactaa

SEQ ID NO: 9 - DNA, 921 bp, synthetic construct
  source 1..921: mol_type = other DNA
aaatacgccctggccgatcctagcctgaagatggctgaccccaaccggttccggggcaagaatctgcctgttctggaccagctgaccgatcctcctggcgtgaaacgggtgtaccacatccagccaagcctggaagatcccttccagcctcctagcatccccatcaccgtgtactacgccgtgctggaaagggcctgtagaagcgtgctgctgcacgccccatctgaagcccctcaaatcgtcagaggcgcttccgacgaggccagaaagcacacctacaacctgacaatcgcctggtacagaatgggcgacaactgcgccattcctatcaccgtgatggagtacaccgagtgtccctacaacaagagcctgggcgtgtgccccatcagaacacagcctagatggtcctactacgacagcttcagcgccgtgtccgaggacaatctgggcttcctgatgcatgcccctgcctttgagacagccggcacctatctgcggctggtcaagatcaacgactggaccgagatcacccagttcatcctggaacacagagccagagccagctgcaaatacgctctgcccctgagaattcctcctgccgcctgtctgacaagcaaggcctatcagcagggcgtgaccgtggatagcatcggcatgctgcccagattcatccccgagaaccagagaacagtggccctgtactccctgaagatcgccggatggcacggacccaagcctccatacacaagcacactgctgcctccagagctgagcgacaccaccaatgccacacagcctgaactggtgcctgaggacccagaggattctgccctgcttgaagatcctgccggcaccgtgtctagccagattcctcctaactggcacatccccagcatccaggatgtggcccctcatcattga

SEQ ID NO: 10 - DNA, 921 bp, synthetic construct
  source 1..921: mol_type = other DNA
aaatatgctctcgctgatccgagcctcaagatggcagatcccaaccgatttcggggaaagaatctgccagtactggaccagctgacggacccacctggcgtcaaacgcgtctaccacatacagcctagtcttgaggacccttttcagccaccgtctatccccattaccgtgtactatgccgtgctggaacgcgcgtgtaggtcagttctgctgcatgccccatccgaagccccccagatcgtcagaggagcttctgatgaagcacgcaaacacacctacaacctcacaatagcgtggtatcgaatgggcgataactgcgcaattcccatcacagtcatggagtacacggagtgcccctacaacaagagcctcggtgtttgccctatcaggacacaacccaggtggagctattacgacagtttcagcgccgtgtctgaggacaatctggggtttctgatgcacgcacccgccttcgagactgccggcacctacttgcggctggtgaagatcaacgactggactgagatcacccagttcatcctggaacatagggccagagccagctgcaagtatgctcttcccctgcggattccgcctgcagcatgtctgacctcaaaagcctaccagcaaggggtgactgtggacagcattggcatgctgcctcgtttcattcccgagaatcaacggacagtggctctgtattccctgaagatcgcaggatggcatgggcccaaaccaccttatacctctacgttgcttccaccagaactcagtgacaccactaatgcgacacagccagaacttgtgcctgaggatcctgaagatagcgctctgttggaggatccagccggtactgtgtcctcccagataccacccaattggcacattccttccattcaggacgtagctccgcatcactga

SEQ ID NO: 11 - DNA, 921 bp, synthetic construct
  source 1..921: mol_type = other DNA
aaatacgccctggccgatccctccctgaaaatggccgatcccaacaggttcagaggcaaaaacctgcccgtgctggatcagctgaccgatccccctggcgtgaaaagagtgtaccacatccagccctccctggaagatcccttccagcccccttccatccccatcaccgtgtactacgccgtgctggaaagagcttgcagatccgtgctgctgcacgccccctccgaagcccctcagatcgtgagaggcgcctccgatgaagccagaaaacacacctacaacctgaccatcgcctggtacagaatgggcgataactgcgccatccccatcaccgtgatggaatacaccgaatgcccctacaacaaatccctgggcgtgtgccccatcagaacccagcccagatggtcctactacgattccttctccgccgtgtccgaagataacctgggcttcctgatgcacgcccccgccttcgaaaccgccggcacctacctgagactggtgaaaatcaacgattggaccgaaatcacccagttcatcctggaacacagagccagagcctcctgcaaatacgccctgcccctgagaatccctcccgccgcctgcctgacctccaaagcctaccagcagggcgtgaccgtggattccatcggcatgctgcccagattcatccccgaaaaccagagaaccgtggccctgtactccctgaaaatcgccggctggcacggccccaaacccccttacacctccaccctgctgccccctgaactgtccgataccaccaacgccacccagcccgaactggtgcccgaagatcccgaagattccgccctgctggaagatcccgccggcaccgtgtcctcccagatccctcccaactggcacatcccctccatccaggatgtggcccctcaccactga

SEQ ID NO: 12 - DNA, 1149 bp, Human alphaherpesvirus 2
  source 1..1149: mol_type = genomic DNA
cgcacctcctggaagcgcgtgacctccggcgaggacgtggtgctgctgcccgcccccgccggccccgaggagcgcacccgcgcccacaagctgctgtgggccgccgagcccctggacgcctgcggccccctgcgcccctcctgggtggccctgtggcccccccgccgcgtgctggagaccgtggtggacgccgcctgcatgcgcgcccccgagcccctggccatcgcctactccccccccttccccgccggcgacgagggcctgtactccgagctggcctggcgcgaccgcgtggccgtggtgaacgagtccctggtgatctacggcgccctggagaccgactccggcctgtacaccctgtccgtggtgggcctgtccgacgaggcccgccaggtggcctccgtggtgctggtggtggagcccgcccccgtgcccacccccacccccgacgactacgacgaggaggacgacgccggcgtgtccgagcgcacccccgtgtccgtgcccccccccacccccccccgccgcccccccgtggccccccccacccacccccgcgtgatccccgaggtgtcccacgtgcgcggcgtgaccgtgcacatggagacccccgaggccatcctgttcgcccccggcgagaccttcggcaccaacgtgtccatccacgccatcgcccacgacgacggcccctacgccatggacgtggtgtggatgcgcttcgacgtgccctcctcctgcgccgagatgcgcatctacgaggcctgcctgtaccacccccagctgcccgagtgcctgtcccccgccgacgccccctgcgccgtgtcctcctgggcctaccgcctggccgtgcgctcctacgccggctgctcccgcaccaccccccccccccgctgcttcgccgaggcccgcatggagcccgtgcccggcctggcctggctggcctccaccgtgaacctggagttccagcacgcctccccccagcacgccggcctgtacctgtgcgtggtgtacgtggacgaccacatccacgcctggggccacatgaccatctccaccgccgcccagtaccgcaacgccgtggtggagcagcacctgccccagcgccagcccgagcccgtggagcccacccgcccccacgtgcgcgcctaa

SEQ ID NO: 13 - DNA, 1149 bp, synthetic construct
  source 1..1149: mol_type = other DNA
agaaccagctggaaaagagtgaccagcggcgaggatgtggtgctgcttcctgctcctgctggccccgaggaaagaacaagagcccacaaactgctgtgggccgctgagcctcttgatgcctgtggacctctcagacctagctgggttgcactgtggccacctcggagagtgctggaaacagtggtggatgccgcctgcatgagagcccctgaacctctggccattgcctactctccaccatttccagccggcgacgagggcctgtattctgagcttgcttggagagacagagtggccgtggtcaacgagagcctggttatctatggcgccctggaaaccgacagcggcctgtacacactgtctgtcgtgggcctgtctgacgaggctagacaggtggcatctgtggtcctggtggtggaacctgctccagtgcctacacctacacctgacgactacgacgaggaagatgacgctggcgtcagcgagagaacccctgtttctgtgcctcctcctacgcctcctcgtagacctcctgttgctcctccaacacaccccagagtgatccctgaagtgtctcacgtgcggggcgtgaccgtgcacatggaaacacctgaggccatcctgttcgcccctggcgagacatttggcaccaacgtgtccatccacgctatcgcccacgacgatggcccttacgccatggatgtcgtgtggatgagattcgacgtgcccagcagctgtgccgagatgagaatctatgaggcctgcctgtatcaccctcagctgcccgaatgtctgagccctgctgatgccccttgtgccgttagcagctgggcctatagactggccgtgcggtcttatgccggctgctctagaacaacccctcctcctcggtgtttcgccgaggccagaatggaacctgttcctggactggcctggctggcctccacagtgaacctggaatttcagcacgcctctccacagcacgccggcctgtatctgtgtgtggtgtacgtggacgatcacatccacgcctggggccacatgaccatctctacagccgctcagtaccggaacgccgtggttgaacagcatctgcctcagagacagcccgagcctgtggaacctacaagacctcatgttcgggcctga

SEQ ID NO: 14 - DNA, 1149 bp, synthetic construct
  source 1..1149: mol_type = other DNA
cgcacctcttggaaacgcgttacttccggggaggacgttgtcctccttccagcacccgcaggacctgaggaaaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaggaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 15 - DNA, 1149 bp, synthetic construct
  source 1..1149: mol_type = other DNA
agaacctcctggaaaagagtgacctccggcgaagatgtggtgctgctgcccgcccccgccggccccgaagaaagaaccagagcccacaaactgctgtgggccgccgaacccctggatgcctgcggccccctcagaccctcctgggtggccctgtggccccctagaagggtgctggaaaccgtggtggatgccgcctgcatgagagcccccgaacccctggccatcgcctactcccctcccttccccgccggcgatgaaggcctgtactccgaactggcctggagagatagagtggccgtggtgaacgaatccctggtgatctacggcgccctggaaaccgattccggcctgtacaccctgtccgtggtgggcctgtccgatgaagccagacaggtggcctccgtggtgctggtggtggaacccgcccccgtgcccacccccacccccgatgattacgatgaagaagatgatgccggcgtgtccgaaagaacccccgtgtccgtgccccctcccacccctccccgcagaccccctgtggcccctcccacccaccccagagtgatccccgaagtgtcccacgtgagaggcgtgaccgtgcacatggaaacccccgaagccatcctgttcgcccccggcgaaaccttcggcaccaacgtgtccatccacgccatcgcccacgatgatggcccctacgccatggatgtggtgtggatgagattcgatgtgccctcctcctgcgccgaaatgagaatctacgaagcctgcctgtaccacccccagctgcccgaatgcctgtcccccgccgatgccccctgcgccgtgtcctcctgggcctacagactggccgtgagatcctacgccggctgctccagaaccacccctccccctagatgcttcgccgaagccagaatggaacccgtgcccggcctggcctggctggcctccaccgtgaacctggaattccagcacgccagcccccagcacgccggcctgtacctgtgcgtggtgtacgtggatgatcacatccacgcctggggccacatgaccatctccaccgccgcccagtacagaaacgccgtggtggaacagcacctgccccagagacagcccgaacccgtggaacccaccagaccccacgtgagagcctga

SEQ ID NO: 16 - RNA, 1203 nt, Human alphaherpesvirus 2
  source 1..1203: mol_type = genomic RNA
gcctcccccggccgcaccatcaccgtgggcccccgcggcaacgcctccaacgccgccccctccgcctccccccgcaacgcctccgccccccgcaccacccccacccccccccagccccgcaaggccaccaagtccaaggcctccaccgccaagcccgcccccccccccaagaccggcccccccaagacctcctccgagcccgtgcgctgcaaccgccacgaccccctggcccgctacggctcccgcgtgcagatccgctgccgcttccccaactccacccgcaccgagttccgcctgcagatctggcgctacgccaccgccaccgacgccgagatcggcaccgccccctccctggaggaggtgatggtgaacgtgtccgccccccccggcggccagctggtgtacgactccgcccccaaccgcaccgacccccacgtgatctgggccgagggcgccggccccggcgcctccccccgcctgtactccgtggtgggccccctgggccgccagcgcctgatcatcgaggagctgaccctggagacccagggcatgtactactgggtgtggggccgcaccgaccgcccctccgcctacggcacctgggtgcgcgtgcgcgtgttccgccccccctccctgaccatccacccccacgccgtgctggagggccagcccttcaaggccacctgcaccgccgccacctactaccccggcaaccgcgccgagttcgtgtggttcgaggacggccgccgcgtgttcgaccccgcccagatccacacccagacccaggagaaccccgacggcttctccaccgtgtccaccgtgacctccgccgccgtgggcggccagggccccccccgcaccttcacctgccagctgacctggcaccgcgactccgtgtccttctcccgccgcaacgcctccggcaccgcctccgtgctgccccgccccaccatcaccatggagttcaccggcgaccacgccgtgtgcaccgccggctgcgtgcccgagggcgtgaccttcgcctggttcctgggcgacgactcctcccccgccgagaaggtggccgtggcctcccagacctcctgcggccgccccggcaccgccaccatccgctccaccctgcccgtgtcctacgagcagaccgagtacatctgccgcctggccggctaccccgacggcatccccgtgctggagcaccactaa

SEQ ID NO: 17 - RNA, 1206 nt, synthetic construct
  source 1..1206: mol_type = other RNA
agcgcttctcccggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggcccaggcgcaagccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactga

SEQ ID NO: 18 - RNA, 1203 nt, synthetic construct
  source 1..1203: mol_type = other RNA
gcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 19 - RNA, 1203 nt, synthetic construct
  source 1..1203: mol_type = other RNA
gcctcccccggcagaaccatcaccgtgggccccagaggcaacgcctccaacgccgccccctccgcctcccccagaaacgcctccgcccccagaaccacccccacccctccccagcccagaaaagccaccaaatccaaagcctccaccgccaaacccgcccctcctcccaaaaccggccctcccaaaacctcctccgaacccgtgagatgcaacagacacgatcccctggccagatacggctccagagtgcagatcagatgcagattccccaactccaccagaaccgaattcagactgcagatctggagatacgccaccgccaccgatgccgaaatcggcaccgccccctccctggaagaagtgatggtgaacgtgtccgcccctcctggcggccagctggtgtacgattccgcccccaacagaaccgatccccacgtgatctgggccgaaggcgccggccccggcgcctcccccagactgtactccgtggtgggccccctgggcagacagagactgatcatcgaagaactgaccctggaaacccagggcatgtactactgggtgtggggcagaaccgatagaccctccgcctacggcacctgggtgagagtgagagtgttcagacccccttccctgaccatccacccccacgccgtgctggaaggccagcccttcaaagccacctgcaccgccgccacctactaccccggcaacagagccgaattcgtgtggttcgaagatggcagaagggtgttcgatcccgcccagatccacacccagacccaggaaaaccccgacggcttctccaccgtgtccaccgtgacctccgccgccgtgggcggccagggccctcccagaaccttcacctgccagctgacctggcacagagactccgtgtccttctccagaagaaacgcctccggcaccgcctccgtgctgcccagacccaccatcaccatggaattcaccggcgatcacgccgtgtgcaccgccggctgcgtgcccgaaggcgtgaccttcgcctggttcctgggcgatgattcctcccccgccgaaaaagtggccgtggcctcccagacctcctgcggcagacccggcaccgccaccatcagatccaccctgcccgtgtcctacgaacagaccgaatacatctgcagactggccggctaccccgatggcatccccgtgctggaacaccactga

SEQ ID NO: 20 - RNA, 921 nt, Human alphaherpesvirus 2
  source 1..921: mol_type = genomic RNA
aagtacgccctggccgacccctccctgaagatggccgaccccaaccgcttccgcggcaagaacctgcccgtgctggaccagctgaccgacccccccggcgtgaagcgcgtgtaccacatccagccctccctggaggaccccttccagcccccctccatccccatcaccgtgtactacgccgtgctggagcgcgcctgccgctccgtgctgctgcacgccccctccgaggccccccagatcgtgcgcggcgcctccgacgaggcccgcaagcacacctacaacctgaccatcgcctggtaccgcatgggcgacaactgcgccatccccatcaccgtgatggagtacaccgagtgcccctacaacaagtccctgggcgtgtgccccatccgcacccagccccgctggtcctactacgactccttctccgccgtgtccgaggacaacctgggcttcctgatgcacgcccccgccttcgagaccgccggcacctacctgcgcctggtgaagatcaacgactggaccgagatcacccagttcatcctggagcaccgcgcccgcgcctcctgcaagtacgccctgcccctgcgcatcccccccgccgcctgcctgacctccaaggcctaccagcagggcgtgaccgtggactccatcggcatgctgccccgcttcatccccgagaaccagcgcaccgtggccctgtactccctgaagatcgccggctggcacggccccaagcccccctacacctccaccctgctgccccccgagctgtccgacaccaccaacgccacccagcccgagctggtgcccgaggaccccgaggactccgccctgctggaggaccccgccggcaccgtgtcctcccagatcccccccaactggcacatcccctccatccaggacgtggccccccaccactaa

SEQ ID NO: 21 - RNA, 921 nt, synthetic construct
  source 1..921: mol_type = other RNA
aaatacgccctggccgatcctagcctgaagatggctgaccccaaccggttccggggcaagaatctgcctgttctggaccagctgaccgatcctcctggcgtgaaacgggtgtaccacatccagccaagcctggaagatcccttccagcctcctagcatccccatcaccgtgtactacgccgtgctggaaagggcctgtagaagcgtgctgctgcacgccccatctgaagcccctcaaatcgtcagaggcgcttccgacgaggccagaaagcacacctacaacctgacaatcgcctggtacagaatgggcgacaactgcgccattcctatcaccgtgatggagtacaccgagtgtccctacaacaagagcctgggcgtgtgccccatcagaacacagcctagatggtcctactacgacagcttcagcgccgtgtccgaggacaatctgggcttcctgatgcatgcccctgcctttgagacagccggcacctatctgcggctggtcaagatcaacgactggaccgagatcacccagttcatcctggaacacagagccagagccagctgcaaatacgctctgcccctgagaattcctcctgccgcctgtctgacaagcaaggcctatcagcagggcgtgaccgtggatagcatcggcatgctgcccagattcatccccgagaaccagagaacagtggccctgtactccctgaagatcgccggatggcacggacccaagcctccatacacaagcacactgctgcctccagagctgagcgacaccaccaatgccacacagcctgaactggtgcctgaggacccagaggattctgccctgcttgaagatcctgccggcaccgtgtctagccagattcctcctaactggcacatccccagcatccaggatgtggcccctcatcattga

SEQ ID NO: 22 - RNA, 921 nt, synthetic construct
  source 1..921: mol_type = other RNA
aaatatgctctcgctgatccgagcctcaagatggcagatcccaaccgatttcggggaaagaatctgccagtactggaccagctgacggacccacctggcgtcaaacgcgtctaccacatacagcctagtcttgaggacccttttcagccaccgtctatccccattaccgtgtactatgccgtgctggaacgcgcgtgtaggtcagttctgctgcatgccccatccgaagccccccagatcgtcagaggagcttctgatgaagcacgcaaacacacctacaacctcacaatagcgtggtatcgaatgggcgataactgcgcaattcccatcacagtcatggagtacacggagtgcccctacaacaagagcctcggtgtttgccctatcaggacacaacccaggtggagctattacgacagtttcagcgccgtgtctgaggacaatctggggtttctgatgcacgcacccgccttcgagactgccggcacctacttgcggctggtgaagatcaacgactggactgagatcacccagttcatcctggaacatagggccagagccagctgcaagtatgctcttcccctgcggattccgcctgcagcatgtctgacctcaaaagcctaccagcaaggggtgactgtggacagcattggcatgctgcctcgtttcattcccgagaatcaacggacagtggctctgtattccctgaagatcgcaggatggcatgggcccaaaccaccttatacctctacgttgcttccaccagaactcagtgacaccactaatgcgacacagccagaacttgtgcctgaggatcctgaagatagcgctctgttggaggatccagccggtactgtgtcctcccagataccacccaattggcacattccttccattcaggacgtagctccgcatcactga

SEQ ID NO: 23 - RNA, 921 nt, synthetic construct
  source 1..921: mol_type = other RNA
aaatacgccctggccgatccctccctgaaaatggccgatcccaacaggttcagaggcaaaaacctgcccgtgctggatcagctgaccgatccccctggcgtgaaaagagtgtaccacatccagccctccctggaagatcccttccagcccccttccatccccatcaccgtgtactacgccgtgctggaaagagcttgcagatccgtgctgctgcacgccccctccgaagcccctcagatcgtgagaggcgcctccgatgaagccagaaaacacacctacaacctgaccatcgcctggtacagaatgggcgataactgcgccatccccatcaccgtgatggaatacaccgaatgcccctacaacaaatccctgggcgtgtgccccatcagaacccagcccagatggtcctactacgattccttctccgccgtgtccgaagataacctgggcttcctgatgcacgcccccgccttcgaaaccgccggcacctacctgagactggtgaaaatcaacgattggaccgaaatcacccagttcatcctggaacacagagccagagcctcctgcaaatacgccctgcccctgagaatccctcccgccgcctgcctgacctccaaagcctaccagcagggcgtgaccgtggattccatcggcatgctgcccagattcatccccgaaaaccagagaaccgtggccctgtactccctgaaaatcgccggctggcacggccccaaacccccttacacctccaccctgctgccccctgaactgtccgataccaccaacgccacccagcccgaactggtgcccgaagatcccgaagattccgccctgctggaagatcccgccggcaccgtgtcctcccagatccctcccaactggcacatcccctccatccaggatgtggcccctcaccactga

SEQ ID NO: 24 - RNA, 1149 nt, Human alphaherpesvirus 2
  source 1..1149: mol_type = genomic RNA
cgcacctcctggaagcgcgtgacctccggcgaggacgtggtgctgctgcccgcccccgccggccccgaggagcgcacccgcgcccacaagctgctgtgggccgccgagcccctggacgcctgcggccccctgcgcccctcctgggtggccctgtggcccccccgccgcgtgctggagaccgtggtggacgccgcctgcatgcgcgcccccgagcccctggccatcgcctactccccccccttccccgccggcgacgagggcctgtactccgagctggcctggcgcgaccgcgtggccgtggtgaacgagtccctggtgatctacggcgccctggagaccgactccggcctgtacaccctgtccgtggtgggcctgtccgacgaggcccgccaggtggcctccgtggtgctggtggtggagcccgcccccgtgcccacccccacccccgacgactacgacgaggaggacgacgccggcgtgtccgagcgcacccccgtgtccgtgcccccccccacccccccccgccgcccccccgtggccccccccacccacccccgcgtgatccccgaggtgtcccacgtgcgcggcgtgaccgtgcacatggagacccccgaggccatcctgttcgcccccggcgagaccttcggcaccaacgtgtccatccacgccatcgcccacgacgacggcccctacgccatggacgtggtgtggatgcgcttcgacgtgccctcctcctgcgccgagatgcgcatctacgaggcctgcctgtaccacccccagctgcccgagtgcctgtcccccgccgacgccccctgcgccgtgtcctcctgggcctaccgcctggccgtgcgctcctacgccggctgctcccgcaccaccccccccccccgctgcttcgccgaggcccgcatggagcccgtgcccggcctggcctggctggcctccaccgtgaacctggagttccagcacgcctccccccagcacgccggcctgtacctgtgcgtggtgtacgtggacgaccacatccacgcctggggccacatgaccatctccaccgccgcccagtaccgcaacgccgtggtggagcagcacctgccccagcgccagcccgagcccgtggagcccacccgcccccacgtgcgcgcctaa

SEQ ID NO: 25 - RNA, 1149 nt, synthetic construct
  source 1..1149: mol_type = other RNA
agaaccagctggaaaagagtgaccagcggcgaggatgtggtgctgcttcctgctcctgctggccccgaggaaagaacaagagcccacaaactgctgtgggccgctgagcctcttgatgcctgtggacctctcagacctagctgggttgcactgtggccacctcggagagtgctggaaacagtggtggatgccgcctgcatgagagcccctgaacctctggccattgcctactctccaccatttccagccggcgacgagggcctgtattctgagcttgcttggagagacagagtggccgtggtcaacgagagcctggttatctatggcgccctggaaaccgacagcggcctgtacacactgtctgtcgtgggcctgtctgacgaggctagacaggtggcatctgtggtcctggtggtggaacctgctccagtgcctacacctacacctgacgactacgacgaggaagatgacgctggcgtcagcgagagaacccctgtttctgtgcctcctcctacgcctcctcgtagacctcctgttgctcctccaacacaccccagagtgatccctgaagtgtctcacgtgcggggcgtgaccgtgcacatggaaacacctgaggccatcctgttcgcccctggcgagacatttggcaccaacgtgtccatccacgctatcgcccacgacgatggcccttacgccatggatgtcgtgtggatgagattcgacgtgcccagcagctgtgccgagatgagaatctatgaggcctgcctgtatcaccctcagctgcccgaatgtctgagccctgctgatgccccttgtgccgttagcagctgggcctatagactggccgtgcggtcttatgccggctgctctagaacaacccctcctcctcggtgtttcgccgaggccagaatggaacctgttcctggactggcctggctggcctccacagtgaacctggaatttcagcacgcctctccacagcacgccggcctgtatctgtgtgtggtgtacgtggacgatcacatccacgcctggggccacatgaccatctctacagccgctcagtaccggaacgccgtggttgaacagcatctgcctcagagacagcccgagcctgtggaacctacaagacctcatgttcgggcctga

SEQ ID NO: 26 - RNA, 1149 nt, synthetic construct
  source 1..1149: mol_type = other RNA
cgcacctcttggaaacgcgttacttccggggaggacgttgtcctccttccagcacccgcaggacctgaggaaaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaggaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 27 - RNA, 1149 nt, synthetic construct
  source 1..1149: mol_type = other RNA
agaacctcctggaaaagagtgacctccggcgaagatgtggtgctgctgcccgcccccgccggccccgaagaaagaaccagagcccacaaactgctgtgggccgccgaacccctggatgcctgcggccccctcagaccctcctgggtggccctgtggccccctagaagggtgctggaaaccgtggtggatgccgcctgcatgagagcccccgaacccctggccatcgcctactcccctcccttccccgccggcgatgaaggcctgtactccgaactggcctggagagatagagtggccgtggtgaacgaatccctggtgatctacggcgccctggaaaccgattccggcctgtacaccctgtccgtggtgggcctgtccgatgaagccagacaggtggcctccgtggtgctggtggtggaacccgcccccgtgcccacccccacccccgatgattacgatgaagaagatgatgccggcgtgtccgaaagaacccccgtgtccgtgccccctcccacccctccccgcagaccccctgtggcccctcccacccaccccagagtgatccccgaagtgtcccacgtgagaggcgtgaccgtgcacatggaaacccccgaagccatcctgttcgcccccggcgaaaccttcggcaccaacgtgtccatccacgccatcgcccacgatgatggcccctacgccatggatgtggtgtggatgagattcgatgtgccctcctcctgcgccgaaatgagaatctacgaagcctgcctgtaccacccccagctgcccgaatgcctgtcccccgccgatgccccctgcgccgtgtcctcctgggcctacagactggccgtgagatcctacgccggctgctccagaaccacccctccccctagatgcttcgccgaagccagaatggaacccgtgcccggcctggcctggctggcctccaccgtgaacctggaattccagcacgccagcccccagcacgccggcctgtacctgtgcgtggtgtacgtggatgatcacatccacgcctggggccacatgaccatctccaccgccgcccagtacagaaacgccgtggtggaacagcacctgccccagagacagcccgaacccgtggaacccaccagaccccacgtgagagcctga

SEQ ID NO: 28 - protein, 19 aa, synthetic construct
MRMQLLLLIALSLALVTNS

SEQ ID NO: 29 - protein, 25 aa, synthetic construct
MGRLTSGVGTAALLVVAVGLRVVCA

SEQ ID NO: 30 - protein, 28 aa, synthetic construct
MGRLTSGVGTAALLVVAVGLRVVCAKYA

SEQ ID NO: 31 - protein, 30 aa, synthetic construct
MGRLTSGVGTAALLVVAVGLRVVCAKYALA

SEQ ID NO: 32 - protein, 27 aa, synthetic construct
MALGRVGLAVGLWGLLWVGVVVVLANA

SEQ ID NO: 33 - protein, 23 aa, synthetic construct
MARGAGLVFFVGVWVVSCLAAAP

SEQ ID NO: 34 - DNA, 57 bp, synthetic construct
  source 1..57: mol_type = other DNA
atgcgcatgcagctgctgctgctgatcgccctgtccctggccctggtgaccaactcc

SEQ ID NO: 35 - DNA, 54 bp, synthetic construct
  source 1..54: mol_type = other DNA
atgagaatgcagctgctgctcctgatcgccctgtctctggccctggtcaccaat

SEQ ID NO: 36 - DNA, 57 bp, synthetic construct
  source 1..57: mol_type = other DNA
atgcgcatgcaactgctcctgctgattgcgttgagccttgccctggtgaccaacagc

SEQ ID NO: 37 - DNA, 57 bp, synthetic construct
  source 1..57: mol_type = other DNA
atgagaatgcagctgctgctgctgatcgccctgtccctggccctggtgaccaactcc

SEQ ID NO: 38 - DNA, 75 bp, synthetic construct
  source 1..75: mol_type = other DNA
atgggccgcctgacctccggcgtgggcaccgccgccctgctggtggtggccgtgggcctgcgcgtggtgtgcgcc

SEQ ID NO: 39 - DNA, 75 bp, synthetic construct
  source 1..75: mol_type = other DNA
atgggcagactgacatctggcgtgggaacagctgctctgctggtggttgctgtgggcctgagagtcgtgtgtgcc

SEQ ID NO: 40 - DNA, 75 bp, synthetic construct
  source 1..75: mol_type = other DNA
atggggagactcacatcaggcgtaggaaccgctgccctgttggtcgtggccgttggtctgagagttgtgtgtgcc

SEQ ID NO: 41 - DNA, 75 bp, synthetic construct
  source 1..75: mol_type = other DNA
atgggcagactgacctccggcgtgggcaccgccgccctgctggtggtggccgtgggcctgagagtggtgtgcgcc

SEQ ID NO: 42 - DNA, 84 bp, synthetic construct
  source 1..84: mol_type = other DNA
atgggccgcctgacctccggcgtgggcaccgccgccctgctggtggtggccgtgggcctgcgcgtggtgtgcgccaagtacgcc

SEQ ID NO: 43 - DNA, 84 bp, synthetic construct
  source 1..84: mol_type = other DNA
atgggcagactgacatctggcgtgggaacagctgctctgctggtggttgctgtgggcctgagagtcgtgtgtgccaaatacgcc

SEQ ID NO: 44 - DNA, 84 bp, synthetic construct
  source 1..84: mol_type = other DNA
atggggagactcacatcaggcgtaggaaccgctgccctgttggtcgtggccgttggtctgagagttgtgtgtgccaaatatgct

SEQ ID NO: 45 - DNA, 84 bp, synthetic construct
  source 1..84: mol_type = other DNA
atgggcagactgacctccggcgtgggcaccgccgccctgctggtggtggccgtgggcctgagagtggtgtgcgccaaatacgcc

SEQ ID NO: 46 - DNA, 90 bp, synthetic construct
  source 1..90: mol_type = other DNA
atgggccgcctgacctccggcgtgggcaccgccgccctgctggtggtggccgtgggcctgcgcgtggtgtgcgccaagtacgccctggcc

SEQ ID NO: 47 - DNA, 90 bp, synthetic construct
  source 1..90: mol_type = other DNA
atgggcagactgacatctggcgtgggaacagctgctctgctggtggttgctgtgggcctgagagtcgtgtgtgccaaatacgccctggcc

SEQ ID NO: 48 - DNA, 87 bp, synthetic construct
  source 1..87: mol_type = other DNA
atggggagactcacatcaggcgtaggaaccgctgccctgttggtcgtggccgttggtctgagagttgtgtgtgccaaatatgctctg

SEQ ID NO: 49 - DNA, 87 bp, synthetic construct
  source 1..87: mol_type = other DNA
atgggcagactgacctccggcgtgggcaccgccgccctgctggtggtggccgtgggcctgagagtggtgtgcgccaaatacgccctg

SEQ ID NO: 50 - RNA, 57 nt, synthetic construct
  source 1..57: mol_type = other RNA
atgcgcatgcagctgctgctgctgatcgccctgtccctggccctggtgaccaactcc

SEQ ID NO: 51 - RNA, 54 nt, synthetic construct
  source 1..54: mol_type = other RNA
atgagaatgcagctgctgctcctgatcgccctgtctctggccctggtcaccaat

SEQ ID NO: 52 - RNA, 57 nt, synthetic construct
  source 1..57: mol_type = other RNA
atgcgcatgcaactgctcctgctgattgcgttgagccttgccctggtgaccaacagc

SEQ ID NO: 53 - RNA, 57 nt, synthetic construct
  source 1..57: mol_type = other RNA
atgagaatgcagctgctgctgctgatcgccctgtccctggccctggtgaccaactcc

SEQ ID NO: 54 - RNA, 75 nt, synthetic construct
  source 1..75: mol_type = other RNA
atgggccgcctgacctccggcgtgggcaccgccgccctgctggtggtggccgtgggcctgcgcgtggtgtgcgcc

SEQ ID NO: 55 - RNA, 75 nt, synthetic construct
  source 1..75: mol_type = other RNA
atgggcagactgacatctggcgtgggaacagctgctctgctggtggttgctgtgggcctgagagtcgtgtgtgcc

SEQ ID NO: 56 - RNA, 75 nt, synthetic construct
  source 1..75: mol_type = other RNA
atggggagactcacatcaggcgtaggaaccgctgccctgttggtcgtggccgttggtctgagagttgtgtgtgcc

SEQ ID NO: 57 - RNA, 75 nt, synthetic construct
  source 1..75: mol_type = other RNA
atgggcagactgacctccggcgtgggcaccgccgccctgctggtggtggccgtgggcctgagagtggtgtgcgcc

SEQ ID NO: 58 - RNA, 84 nt, synthetic construct
  source 1..84: mol_type = other RNA
atgggccgcctgacctccggcgtgggcaccgccgccctgctggtggtggccgtgggcctgcgcgtggtgtgcgccaagtacgcc

SEQ ID NO: 59 - RNA, 84 nt, synthetic construct
  source 1..84: mol_type = other RNA
atggggagactcacatcaggcgtaggaaccgctgccctgttggtcgtggccgttggtctgagagttgtgtgtgccaaatatgct

SEQ ID NO: 60 - RNA, 84 nt, synthetic construct; identical to SEQ ID NO: 59
  source 1..84: mol_type = other RNA

SEQ ID NO: 61 - RNA, 90 nt, synthetic construct
  source 1..90: mol_type = other RNA
atgggccgcctgacctccggcgtgggcaccgccgccctgctggtggtggccgtgggcctgcgcgtggtgtgcgccaagtacgccctggcc

SEQ ID NO: 62 - RNA, 90 nt, synthetic construct
  source 1..90: mol_type = other RNA
atgggcagactgacatctggcgtgggaacagctgctctgctggtggttgctgtgggcctgagagtcgtgtgtgccaaatacgccctggcc

SEQ ID NO: 63 - RNA, 87 nt, synthetic construct
  source 1..87: mol_type = other RNA
atggggagactcacatcaggcgtaggaaccgctgccctgttggtcgtggccgttggtctgagagttgtgtgtgccaaatatgctctg

SEQ ID NO: 64 - RNA, 87 nt, synthetic construct
  source 1..87: mol_type = other RNA
atgggcagactgacctccggcgtgggcaccgccgccctgctggtggtggccgtgggcctgagagtggtgtgcgccaaatacgccctg

SEQ ID NO: 65 - protein, 419 aa, synthetic construct
MRMQLLLLIALSLALVTNSASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 66 - protein, 425 aa, synthetic construct
MGRLTSGVGTAALLVVAVGLRVVCAASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 67 - protein, 428 aa, synthetic construct
MGRLTSGVGTAALLVVAVGLRVVCAKYAASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 68 - protein, 429 aa, synthetic construct
MGRLTSGVGTAALLVVAVGLRVVCAKYALASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 69 - protein, 321 aa, synthetic construct
MRMQLLLLIALSLALVTNSADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHH

SEQ ID NO: 70 - protein, 331 aa, synthetic construct
MGRLTSGVGTAALLVVAVGLRVVCAKYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHH

SEQ ID NO: 71 - protein, 329 aa, synthetic construct
MGRLTSGVGTAALLVVAVGLRVVCAKYADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHH

SEQ ID NO: 72 - protein, 25 aa, synthetic construct
MAISGVPVLGFFIIAVLMSAQESWA

SEQ ID NO: 73 - protein, 401 aa, synthetic construct
MRMQLLLLIALSLALVTNSRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRA

SEQ ID NO: 74 - protein, 407 aa, synthetic construct
MGRLTSGVGTAALLVVAVGLRVVCARTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRA

SEQ ID NO: 75 - protein, 410 aa, synthetic construct
MGRLTSGVGTAALLVVAVGLRVVCAKYARTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRA

SEQ ID NO: 76 - protein, 412 aa, synthetic construct
MGRLTSGVGTAALLVVAVGLRVVCAKYALARTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRA

SEQ ID NO: 77 - DNA, 1260 bp, synthetic construct
  source 1..1260: mol_type = other DNA
atgcgcatgcagctgctgctgctgatcgccctgtccctggccctggtgaccaactccgcctcccccggccgcaccatcaccgtgggcccccgcggcaacgcctccaacgccgccccctccgcctccccccgcaacgcctccgccccccgcaccacccccacccccccccagccccgcaaggccaccaagtccaaggcctccaccgccaagcccgcccccccccccaagaccggcccccccaagacctcctccgagcccgtgcgctgcaaccgccacgaccccctggcccgctacggctcccgcgtgcagatccgctgccgcttccccaactccacccgcaccgagttccgcctgcagatctggcgctacgccaccgccaccgacgccgagatcggcaccgccccctccctggaggaggtgatggtgaacgtgtccgccccccccggcggccagctggtgtacgactccgcccccaaccgcaccgacccccacgtgatctgggccgagggcgccggccccggcgcctccccccgcctgtactccgtggtgggccccctgggccgccagcgcctgatcatcgaggagctgaccctggagacccagggcatgtactactgggtgtggggccgcaccgaccgcccctccgcctacggcacctgggtgcgcgtgcgcgtgttccgccccccctccctgaccatccacccccacgccgtgctggagggccagcccttcaaggccacctgcaccgccgccacctactaccccggcaaccgcgccgagttcgtgtggttcgaggacggccgccgcgtgttcgaccccgcccagatccacacccagacccaggagaaccccgacggcttctccaccgtgtccaccgtgacctccgccgccgtgggcggccagggccccccccgcaccttcacctgccagctgacctggcaccgcgactccgtgtccttctcccgccgcaacgcctccggcaccgcctccgtgctgccccgccccaccatcaccatggagttcaccggcgaccacgccgtgtgcaccgccggctgcgtgcccgagggcgtgaccttcgcctggttcctgggcgacgactcctcccccgccgagaaggtggccgtggcctcccagacctcctgcggccgccccggcaccgccaccatccgctccaccctgcccgtgtcctacgagcagaccgagtacatctgccgcctggccggctaccccgacggcatccccgtgctggagcaccactaa

SEQ ID NO: 78 - DNA, 1260 bp, synthetic construct
  source 1..1260: mol_type = other DNA
atgagaatgcagctgctgctcctgatcgccctgtctctggccctggtcaccaatagcgcttctcccggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggcccaggcgcaagccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactga

SEQ ID NO: 79 - DNA, 1260 bp, synthetic construct
  source 1..1260: mol_type = other DNA
atgcgcatgcaactgctcctgctgattgcgttgagccttgccctggtgaccaacagcgcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 80 - DNA, 1260 bp, synthetic construct
  source 1..1260: mol_type = other DNA
atgagaatgcagctgctgctgctgatcgccctgtccctggccctggtgaccaactccgcctcccccggcagaaccatcaccgtgggccccagaggcaacgcctccaacgccgccccctccgcctcccccagaaacgcctccgcccccagaaccacccccacccctccccagcccagaaaagccaccaaatccaaagcctccaccgccaaacccgcccctcctcccaaaaccggccctcccaaaacctcctccgaacccgtgagatgcaacagacacgatcccctggccagatacggctccagagtgcagatcagatgcagattccccaactccaccagaaccgaattcagactgcagatctggagatacgccaccgccaccgatgccgaaatcggcaccgccccctccctggaagaagtgatggtgaacgtgtccgcccctcctggcggccagctggtgtacgattccgcccccaacagaaccgatccccacgtgatctgggccgaaggcgccggccccggcgcctcccccagactgtactccgtggtgggccccctgggcagacagagactgatcatcgaagaactgaccctggaaacccagggcatgtactactgggtgtggggcagaaccgatagaccctccgcctacggcacctgggtgagagtgagagtgttcagacccccttccctgaccatccacccccacgccgtgctggaaggccagcccttcaaagccacctgcaccgccgccacctactaccccggcaacagagccgaattcgtgtggttcgaagatggcagaagggtgttcgatcccgcccagatccacacccagacccaggaaaaccccgacggcttctccaccgtgtccaccgtgacctccgccgccgtgggcggccagggccctcccagaaccttcacctgccagctgacctggcacagagactccgtgtccttctccagaagaaacgcctccggcaccgcctccgtgctgcccagacccaccatcaccatggaattcaccggcgatcacgccgtgtgcaccgccggctgcgtgcccgaaggcgtgaccttcgcctggttcctgggcgatgattcctcccccgccgaaaaagtggccgtggcctcccagacctcctgcggcagacccggcaccgccaccatcagatccaccctgcccgtgtcctacgaacagaccgaatacatctgcagactggccggctaccccgatggcatccccgtgctggaacaccactga

SEQ ID NO: 81 - DNA, 1287 bp, synthetic construct
  source 1..1287: mol_type = other DNA
atgggccgcctgacctccggcgtgggcaccgccgccctgctggtggtggccgtgggcctgcgcgtggtgtgcgccaagtacgccgcctcccccggccgcaccatcaccgtgggcccccgcggcaacgcctccaacgccgccccctccgcctccccccgcaacgcctccgccccccgcaccacccccacccccccccagccccgcaaggccaccaagtccaaggcctccaccgccaagcccgcccccccccccaagaccggcccccccaagacctcctccgagcccgtgcgctgcaaccgccacgaccccctggcccgctacggctcccgcgtgcagatccgctgccgcttccccaactccacccgcaccgagttccgcctgcagatctggcgctacgccaccgccaccgacgccgagatcggcaccgccccctccctggaggaggtgatggtgaacgtgtccgccccccccggcggccagctggtgtacgactccgcccccaaccgcaccgacccccacgtgatctgggccgagggcgccggccccggcgcctccccccgcctgtactccgtggtgggccccctgggccgccagcgcctgatcatcgaggagctgaccctggagacccagggcatgtactactgggtgtggggccgcaccgaccgcccctccgcctacggcacctgggtgcgcgtgcgcgtgttccgccccccctccctgaccatccacccccacgccgtgctggagggccagcccttcaaggccacctgcaccgccgccacctactaccccggcaaccgcgccgagttcgtgtggttcgaggacggccgccgcgtgttcgaccccgcccagatccacacccagacccaggagaaccccgacggcttctccaccgtgtccaccgtgacctccgccgccgtgggcggccagggccccccccgcaccttcacctgccagctgacctggcaccgcgactccgtgtccttctcccgccgcaacgcctccggcaccgcctccgtgctgccccgccccaccatcaccatggagttcaccggcgaccacgccgtgtgcaccgccggctgcgtgcccgagggcgtgaccttcgcctggttcctgggcgacgactcctcccccgccgagaaggtggccgtggcctcccagacctcctgcggccgccccggcaccgccaccatccgctccaccctgcccgtgtcctacgagcagaccgagtacatctgccgcctggccggctaccccgacggcatccccgtgctggagcaccactaa

SEQ ID NO: 82 - DNA, 1287 bp, synthetic construct
  source 1..1287: mol_type = other DNA
atggggagactcacatcaggcgtaggaaccgctgccctgttggtcgtggccgttggtctgagagttgtgtgtgccaaatatgctgcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 83 - DNA, 1287 bp, synthetic construct
  source 1..1287: mol_type = other DNA
atggggagactcacatcaggcgtaggaaccgctgccctgttggtcgtggccgttggtctgagagttgtgtgtgccaaatatgctgcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 84 - DNA, 1287 bp, synthetic construct; identical to SEQ ID NO: 82
  source 1..1287: mol_type = other DNA

SEQ ID NO: 85 - DNA, 1290 bp, synthetic construct
  source 1..1290: mol_type = other DNA
atgggcagactgacatctggcgtgggaacagctgctctgctggtggttgctgtgggcctgagagtcgtgtgtgccaaatacgccctggcctctcccggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggcccaggcgcaagccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactga

SEQ ID NO: 86 - DNA, 1290 bp, synthetic construct
  source 1..1290: mol_type = other DNA
atggggagactcacatcaggcgtaggaaccgctgccctgttggtcgtggccgttggtctgagagttgtgtgtgccaaatatgctctggcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 87 - DNA, 1290 bp, synthetic construct
  source 1..1290: mol_type = other DNA
atgggcagactgacctccggcgtgggcaccgccgccctgctggtggtggccgtgggcctgagagtggtgtgcgccaaatacgccctggcctcccccggcagaaccatcaccgtgggccccagaggcaacgcctccaacgccgccccctccgcctcccccagaaacgcctccgcccccagaaccacccccacccctccccagcccagaaaagccaccaaatccaaagcctccaccgccaaacccgcccctcctcccaaaaccggccctcccaaaacctcctccgaacccgtgagatgcaacagacacgatcccctggccagatacggctccagagtgcagatcagatgcagattccccaactccaccagaaccgaattcagactgcagatctggagatacgccaccgccaccgatgccgaaatcggcaccgccccctccctggaagaagtgatggtgaacgtgtccgcccctcctggcggccagctggtgtacgattccgcccccaacagaaccgatccccacgtgatctgggccgaaggcgccggccccggcgcctcccccagactgtactccgtggtgggccccctgggcagacagagactgatcatcgaagaactgaccctggaaacccagggcatgtactactgggtgtggggcagaaccgatagaccctccgcctacggcacctgggtgagagtgagagtgttcagacccccttccctgaccatccacccccacgccgtgctggaaggccagcccttcaaagccacctgcaccgccgccacctactaccccggcaacagagccgaattcgtgtggttcgaagatggcagaagggtgttcgatcccgcccagatccacacccagacccaggaaaaccccgacggcttctccaccgtgtccaccgtgacctccgccgccgtgggcggccagggccctcccagaaccttcacctgccagctgacctggcacagagactccgtgtccttctccagaagaaacgcctccggcaccgcctccgtgctgcccagacccaccatcaccatggaattcaccggcgatcacgccgtgtgcaccgccggctgcgtgcccgaaggcgtgaccttcgcctggttcctgggcgatgattcctcccccgccgaaaaagtggccgtggcctcccagacctcctgcggcagacccggcaccgccaccatcagatccaccctgcccgtgtcctacgaacagaccgaatacatctgcagactggccggctaccccgatggcatccccgtgctggaacaccactga

SEQ ID NO: 88 - DNA, 1281 bp, synthetic construct
  source 1..1281: mol_type = other DNA
atggccttggggagagtgggccttgcagtgggtctgtggggactgctctgggttggcgtagtcgtcgtgctggctaacgcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 89 - DNA, 996 bp, synthetic construct
  source 1..996: mol_type = other DNA
atgggccgcctgacctccggcgtgggcaccgccgccctgctggtggtggccgtgggcctgcgcgtggtgtgcgccaagtacgccctggccgacccctccctgaagatggccgaccccaaccgcttccgcggcaagaacctgcccgtgctggaccagctgaccgacccccccggcgtgaagcgcgtgtaccacatccagccctccctggaggaccccttccagcccccctccatccccatcaccgtgtactacgccgtgctggagcgcgcctgccgctccgtgctgctgcacgccccctccgaggccccccagatcgtgcgcggcgcctccgacgaggcccgcaagcacacctacaacctgaccatcgcctggtaccgcatgggcgacaactgcgccatccccatcaccgtgatggagtacaccgagtgcccctacaacaagtccctgggcgtgtgccccatccgcacccagccccgctggtcctactacgactccttctccgccgtgtccgaggacaacctgggcttcctgatgcacgcccccgccttcgagaccgccggcacctacctgcgcctggtgaagatcaacgactggaccgagatcacccagttcatcctggagcaccgcgcccgcgcctcctgcaagtacgccctgcccctgcgcatcccccccgccgcctgcctgacctccaaggcctaccagcagggcgtgaccgtggactccatcggcatgctgccccgcttcatccccgagaaccagcgcaccgtggccctgtactccctgaagatcgccggctggcacggccccaagcccccctacacctccaccctgctgccccccgagctgtccgacaccaccaacgccacccagcccgagctggtgcccgaggaccccgaggactccgccctgctggaggaccccgccggcaccgtgtcctcccagatcccccccaactggcacatcccctccatccaggacgtggccccccaccactaa

SEQ ID NO: 90 - DNA, 996 bp, synthetic construct
  source 1..996: mol_type = other DNA
atgggcagactgacatctggcgtgggaacagctgctctgctggtggttgctgtgggcctgagagtcgtgtgtgccaaatacgccctggccgatcctagcctgaagatggctgaccccaaccggttccggggcaagaatctgcctgttctggaccagctgaccgatcctcctggcgtgaaacgggtgtaccacatccagccaagcctggaagatcccttccagcctcctagcatccccatcaccgtgtactacgccgtgctggaaagggcctgtagaagcgtgctgctgcacgccccatctgaagcccctcaaatcgtcagaggcgcttccgacgaggccagaaagcacacctacaacctgacaatcgcctggtacagaatgggcgacaactgcgccattcctatcaccgtgatggagtacaccgagtgtccctacaacaagagcctgggcgtgtgccccatcagaacacagcctagatggtcctactacgacagcttcagcgccgtgtccgaggacaatctgggcttcctgatgcatgcccctgcctttgagacagccggcacctatctgcggctggtcaagatcaacgactggaccgagatcacccagttcatcctggaacacagagccagagccagctgcaaatacgctctgcccctgagaattcctcctgccgcctgtctgacaagcaaggcctatcagcagggcgtgaccgtggatagcatcggcatgctgcccagattcatccccgagaaccagagaacagtggccctgtactccctgaagatcgccggatggcacggacccaagcctccatacacaagcacactgctgcctccagagctgagcgacaccaccaatgccacacagcctgaactggtgcctgaggacccagaggattctgccctgcttgaagatcctgccggcaccgtgtctagccagattcctcctaactggcacatccccagcatccaggatgtggcccctcatcattga

SEQ ID NO: 91 - DNA, 996 bp, synthetic construct
  source 1..996: mol_type = other DNA
atggggagactcacatcaggcgtaggaaccgctgccctgttggtcgtggccgttggtctgagagttgtgtgtgccaaatatgctctcgctgatccgagcctcaagatggcagatcccaaccgatttcggggaaagaatctgccagtactggaccagctgacggacccacctggcgtcaaacgcgtctaccacatacagcctagtcttgaggacccttttcagccaccgtctatccccattaccgtgtactatgccgtgctggaacgcgcgtgtaggtcagttctgctgcatgccccatccgaagccccccagatcgtcagaggagcttctgatgaagcacgcaaacacacctacaacctcacaatagcgtggtatcgaatgggcgataactgcgcaattcccatcacagtcatggagtacacggagtgcccctacaacaagagcctcggtgtttgccctatcaggacacaacccaggtggagctattacgacagtttcagcgccgtgtctgaggacaatctggggtttctgatgcacgcacccgccttcgagactgccggcacctacttgcggctggtgaagatcaacgactggactgagatcacccagttcatcctggaacatagggccagagccagctgcaagtatgctcttcccctgcggattccgcctgcagcatgtctgacctcaaaagcctaccagcaaggggtgactgtggacagcattggcatgctgcctcgtttcattcccgagaatcaacggacagtggctctgtattccctgaagatcgcaggatggcatgggcccaaaccaccttatacctctacgttgcttccaccagaactcagtgacaccactaatgcgacacagccagaacttgtgcctgaggatcctgaagatagcgctctgttggaggatccagccggtactgtgtcctcccagataccacccaattggcacattccttccattcaggacgtagctccgcatcactga

SEQ ID NO: 92 - DNA, 996 bp, synthetic construct
  source 1..996: mol_type = other DNA
atggggagactcacatcaggcgtaggaaccgctgccctgttggtcgtggccgttggtctgagagttgtgtgtgccaaatatgctctcgctgatccgagcctcaagatggcagatcccaaccgatttcggggaaagaatctgccagtactggaccagctgacggacccacctggcgtcaaacgcgtctaccacatacagcctagtcttgaggacccttttcagccaccgtctatccccattaccgtgtactatgccgtgctggaacgcgcgtgtaggtcagttctgctgcatgccccatccgaagccccccagatcgtcagaggagcttctgatgaagcacgcaaacacacctacaacctcacaatagcgtggtatcgaatgggcgataactgcgcaattcccatcacagtcatggagtacacggagtgcccctacaacaagagcctcggtgtttgccctatcaggacacaacccaggtggagctattacgacagtttcagcgccgtgtctgaggacaatctggggtttctgatgcacgcacccgccttcgagactgccggcacctacttgcggctggtgaagatcaacgactggactgagatcacccagttcatcctggaacatagggccagagccagctgcaagtatgcccttcccctgcggattccgcctgcagcatgtctgacctcaaaagcctaccagcaaggggtgactgtggacagcattggcatgctgcctcgtttcattcccgagaatcaacggacagtggctctgtattccctgaagatcgcaggatggcatgggcccaaaccaccttatacctctacgttgcttccaccagaactcagtgacaccactaatgcgacacagccagaacttgtgcctgaggatcctgaagatagcgctctgttggaggatccagccggtactgtgtcctcccagataccacccaattggcacattccttccattcaggacgtagctccgcatcactga

SEQ ID NO: 93 - DNA, 996 bp, synthetic construct
  source 1..996: mol_type = other DNA
atgggcagactgacctccggcgtgggcaccgccgccctgctggtggtggccgtgggcctgagagtggtgtgcgccaaatacgccctggccgatccctccctgaaaatggccgatcccaacaggttcagaggcaaaaacctgcccgtgctggatcagctgaccgatccccctggcgtgaaaagagtgtaccacatccagccctccctggaagatcccttccagcccccttccatccccatcaccgtgtactacgccgtgctggaaagagcttgcagatccgtgctgctgcacgccccctccgaagcccctcagatcgtgagaggcgcctccgatgaagccagaaaacacacctacaacctgaccatcgcctggtacagaatgggcgataactgcgccatccccatcaccgtgatggaatacaccgaatgcccctacaacaaatccctgggcgtgtgccccatcagaacccagcccagatggtcctactacgattccttctccgccgtgtccgaagataacctgggcttcctgatgcacgcccccgccttcgaaaccgccggcacctacctgagactggtgaaaatcaacgattggaccgaaatcacccagttcatcctggaacacagagccagagcctcctgcaaatacgccctgcccctgagaatccctcccgccgcctgcctgacctccaaagcctaccagcagggcgtgaccgtggattccatcggcatgctgcccagattcatccccgaaaaccagagaaccgtggccctgtactccctgaaaatcgccggctggcacggccccaaacccccttacacctccaccctgctgccccctgaactgtccgataccaccaacgccacccagcccgaactggtgcccgaagatcccgaagattccgccctgctggaagatcccgccggcaccgtgtcctcccagatccctcccaactggcacatcccctccatccaggatgtggcccctcaccactga

SEQ ID NO: 94 - DNA, 1206 bp, synthetic construct
  source 1..1206: mol_type = other DNA
atgcgcatgcagctgctgctgctgatcgccctgtccctggccctggtgaccaactcccgcacctcctggaagcgcgtgacctccggcgaggacgtggtgctgctgcccgcccccgccggccccgaggagcgcacccgcgcccacaagctgctgtgggccgccgagcccctggacgcctgcggccccctgcgcccctcctgggtggccctgtggcccccccgccgcgtgctggagaccgtggtggacgccgcctgcatgcgcgcccccgagcccctggccatcgcctactccccccccttccccgccggcgacgagggcctgtactccgagctggcctggcgcgaccgcgtggccgtggtgaacgagtccctggtgatctacggcgccctggagaccgactccggcctgtacaccctgtccgtggtgggcctgtccgacgaggcccgccaggtggcctccgtggtgctggtggtggagcccgcccccgtgcccacccccacccccgacgactacgacgaggaggacgacgccggcgtgtccgagcgcacccccgtgtccgtgcccccccccacccccccccgccgcccccccgtggccccccccacccacccccgcgtgatccccgaggtgtcccacgtgcgcggcgtgaccgtgcacatggagacccccgaggccatcctgttcgcccccggcgagaccttcggcaccaacgtgtccatccacgccatcgcccacgacgacggcccctacgccatggacgtggtgtggatgcgcttcgacgtgccctcctcctgcgccgagatgcgcatctacgaggcctgcctgtaccacccccagctgcccgagtgcctgtcccccgccgacgccccctgcgccgtgtcctcctgggcctaccgcctggccgtgcgctcctacgccggctgctcccgcaccaccccccccccccgctgcttcgccgaggcccgcatggagcccgtgcccggcctggcctggctggcctccaccgtgaacctggagttccagcacgcctccccccagcacgccggcctgtacctgtgcgtggtgtacgtggacgaccacatccacgcctggggccacatgaccatctccaccgccgcccagtaccgcaacgccgtggtggagcagcacctgccccagcgccagcccgagcccgtggagcccacccgcccccacgtgcgcgcctaa

SEQ ID NO: 95 - DNA, 1206 bp, synthetic construct
  source 1..1206: mol_type = other DNA
atgagaatgcagctgctgctcctgatcgccctgtctctggccctggtcaccaatagcagaaccagctggaaaagagtgaccagcggcgaggatgtggtgctgcttcctgctcctgctggccccgaggaaagaacaagagcccacaaactgctgtgggccgctgagcctcttgatgcctgtggacctctcagacctagctgggttgcactgtggccacctcggagagtgctggaaacagtggtggatgccgcctgcatgagagcccctgaacctctggccattgcctactctccaccatttccagccggcgacgagggcctgtattctgagcttgcttggagagacagagtggccgtggtcaacgagagcctggttatctatggcgccctggaaaccgacagcggcctgtacacactgtctgtcgtgggcctgtctgacgaggctagacaggtggcatctgtggtcctggtggtggaacctgctccagtgcctacacctacacctgacgactacgacgaggaagatgacgctggcgtcagcgagagaacccctgtttctgtgcctcctcctacgcctcctcgtagacctcctgttgctcctccaacacaccccagagtgatccctgaagtgtctcacgtgcggggcgtgaccgtgcacatggaaacacctgaggccatcctgttcgcccctggcgagacatttggcaccaacgtgtccatccacgctatcgcccacgacgatggcccttacgccatggatgtcgtgtggatgagattcgacgtgcccagcagctgtgccgagatgagaatctatgaggcctgcctgtatcaccctcagctgcccgaatgtctgagccctgctgatgccccttgtgccgttagcagctgggcctatagactggccgtgcggtcttatgccggctgctctagaacaacccctcctcctcggtgtttcgccgaggccagaatggaacctgttcctggactggcctggctggcctccacagtgaacctggaatttcagcacgcctctccacagcacgccggcctgtatctgtgtgtggtgtacgtggacgatcacatccacgcctggggccacatgaccatctctacagccgctcagtaccggaacgccgtggttgaacagcatctgcctcagagacagcccgagcctgtggaacctacaagacctcatgttcgggcctga

SEQ ID NO: 96 - DNA, 1206 bp, synthetic construct
  source 1..1206: mol_type = other DNA
atgcgaatgcagcttctgctgctcattgccttgtcccttgccttggtgaccaactcacgcacctcttggaaacgcgttacttccggggaggacgttgtcctccttccagcacccgcaggacctgaagagaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaagaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 97 - DNA, 1206 bp, synthetic construct
  source 1..1206: mol_type = other DNA
atgagaatgcagctgctgctgctgatcgccctgtccctggccctggtgaccaactccagaacctcctggaaaagagtgacctccggcgaagatgtggtgctgctgcccgcccccgccggccccgaagaaagaaccagagcccacaaactgctgtgggccgccgaacccctggatgcctgcggccccctcagaccctcctgggtggccctgtggccccctagaagggtgctggaaaccgtggtggatgccgcctgcatgagagcccccgaacccctggccatcgcctactcccctcccttccccgccggcgatgaaggcctgtactccgaactggcctggagagatagagtggccgtggtgaacgaatccctggtgatctacggcgccctggaaaccgattccggcctgtacaccctgtccgtggtgggcctgtccgatgaagccagacaggtggcctccgtggtgctggtggtggaacccgcccccgtgcccacccccacccccgatgattacgatgaagaagatgatgccggcgtgtccgaaagaacccccgtgtccgtgccccctcccacccctccccgcagaccccctgtggcccctcccacccaccccagagtgatccccgaagtgtcccacgtgagaggcgtgaccgtgcacatggaaacccccgaagccatcctgttcgcccccggcgaaaccttcggcaccaacgtgtccatccacgccatcgcccacgatgatggcccctacgccatggatgtggtgtggatgagattcgatgtgccctcctcctgcgccgaaatgagaatctacgaagcctgcctgtaccacccccagctgcccgaatgcctgtcccccgccgatgccccctgcgccgtgtcctcctgggcctacagactggccgtgagatcctacgccggctgctccagaaccacccctccccctagatgcttcgccgaagccagaatggaacccgtgcccggcctggcctggctggcctccaccgtgaacctggaattccagcacgccagcccccagcacgccggcctgtacctgtgcgtggtgtacgtggatgatcacatccacgcctggggccacatgaccatctccaccgccgcccagtacagaaacgccgtggtggaacagcacctgccccagagacagcccgaacccgtggaacccaccagaccccacgtgagagcctga

SEQ ID NO: 98 - DNA, 1233 bp, synthetic construct
  source 1..1233: mol_type = other DNA
atgggccgcctgacctccggcgtgggcaccgccgccctgctggtggtggccgtgggcctgcgcgtggtgtgcgccaagtacgcccgcacctcctggaagcgcgtgacctccggcgaggacgtggtgctgctgcccgcccccgccggccccgaggagcgcacccgcgcccacaagctgctgtgggccgccgagcccctggacgcctgcggccccctgcgcccctcctgggtggccctgtggcccccccgccgcgtgctggagaccgtggtggacgccgcctgcatgcgcgcccccgagcccctggccatcgcctactccccccccttccccgccggcgacgagggcctgtactccgagctggcctggcgcgaccgcgtggccgtggtgaacgagtccctggtgatctacggcgccctggagaccgactccggcctgtacaccctgtccgtggtgggcctgtccgacgaggcccgccaggtggcctccgtggtgctggtggtggagcccgcccccgtgcccacccccacccccgacgactacgacgaggaggacgacgccggcgtgtccgagcgcacccccgtgtccgtgcccccccccacccccccccgccgcccccccgtggccccccccacccacccccgcgtgatccccgaggtgtcccacgtgcgcggcgtgaccgtgcacatggagacccccgaggccatcctgttcgcccccggcgagaccttcggcaccaacgtgtccatccacgccatcgcccacgacgacggcccctacgccatggacgtggtgtggatgcgcttcgacgtgccctcctcctgcgccgagatgcgcatctacgaggcctgcctgtaccacccccagctgcccgagtgcctgtcccccgccgacgccccctgcgccgtgtcctcctgggcctaccgcctggccgtgcgctcctacgccggctgctcccgcaccaccccccccccccgctgcttcgccgaggcccgcatggagcccgtgcccggcctggcctggctggcctccaccgtgaacctggagttccagcacgcctccccccagcacgccggcctgtacctgtgcgtggtgtacgtggacgaccacatccacgcctggggccacatgaccatctccaccgccgcccagtaccgcaacgccgtggtggagcagcacctgccccagcgccagcccgagcccgtggagcccacccgcccccacgtgcgcgcctaa

SEQ ID NO: 99 - DNA, 1233 bp, synthetic construct
  source 1..1233: mol_type = other DNA
atgggcagactgacatctggcgtgggaacagctgctctgctggtggttgctgtgggcctgagagtcgtgtgtgccaaatacgccagaaccagctggaaaagagtgaccagcggcgaggatgtggtgctgcttcctgctcctgctggccccgaggaaagaacaagagcccacaaactgctgtgggccgctgagcctcttgatgcctgtggacctctcagacctagctgggttgcactgtggccacctcggagagtgctggaaacagtggtggatgccgcctgcatgagagcccctgaacctctggccattgcctactctccaccatttccagccggcgacgagggcctgtattctgagcttgcttggagagacagagtggccgtggtcaacgagagcctggttatctatggcgccctggaaaccgacagcggcctgtacacactgtctgtcgtgggcctgtctgacgaggctagacaggtggcatctgtggtcctggtggtggaacctgctccagtgcctacacctacacctgacgactacgacgaggaagatgacgctggcgtcagcgagagaacccctgtttctgtgcctcctcctacgcctcctcgtagacctcctgttgctcctccaacacaccccagagtgatccctgaagtgtctcacgtgcggggcgtgaccgtgcacatggaaacacctgaggccatcctgttcgcccctggcgagacatttggcaccaacgtgtccatccacgctatcgcccacgacgatggcccttacgccatggatgtcgtgtggatgagattcgacgtgcccagcagctgtgccgagatgagaatctatgaggcctgcctgtatcaccctcagctgcccgaatgtctgagccctgctgatgccccttgtgccgttagcagctgggcctatagactggccgtgcggtcttatgccggctgctctagaacaacccctcctcctcggtgtttcgccgaggccagaatggaacctgttcctggactggcctggctggcctccacagtgaacctggaatttcagcacgcctctccacagcacgccggcctgtatctgtgtgtggtgtacgtggacgatcacatccacgcctggggccacatgaccatctctacagccgctcagtaccggaacgccgtggttgaacagcatctgcctcagagacagcccgagcctgtggaacctacaagacctcatgttcgggcctga

SEQ ID NO: 100 - DNA, 1233 bp, synthetic construct
  source 1..1233: mol_type = other DNA
atggggagactcacatcaggcgtaggaaccgctgccctgttggtcgtggccgttggtctgagagttgtgtgtgccaaatatgctcgcacctcttggaaacgcgttacttccggggaggacgttgtcctccttccagcacccgcaggacctgaggaaaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaggaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 101 - DNA, 1233 bp, synthetic construct
  source 1..1233: mol_type = other DNA
atggggagactcacatcaggcgtaggaaccgctgccctgttggtcgtggccgttggtctgagagttgtgtgtgccaaatatgctcgcacctcttggaaacgcgttacttccggggaggacgttgtcctccttccagcacccgcaggacctgaagagaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaagaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 102 - DNA, 1233 bp, synthetic construct
  source 1..1233: mol_type = other DNA
atgggcagactgacctccggcgtgggcaccgccgccctgctggtggtggccgtgggcctgagagtggtgtgcgccaaatacgccagaacctcctggaaaagagtgacctccggcgaagatgtggtgctgctgcccgcccccgccggccccgaagaaagaaccagagcccacaaactgctgtgggccgccgaacccctggatgcctgcggccccctcagaccctcctgggtggccctgtggccccctagaagggtgctggaaaccgtggtggatgccgcctgcatgagagcccccgaacccctggccatcgcctactcccctcccttccccgccggcgatgaaggcctgtactccgaactggcctggagagatagagtggccgtggtgaacgaatccctggtgatctacggcgccctggaaaccgattccggcctgtacaccctgtccgtggtgggcctgtccgatgaagccagacaggtggcctccgtggtgctggtggtggaacccgcccccgtgcccacccccacccccgatgattacgatgaagaagatgatgccggcgtgtccgaaagaacccccgtgtccgtgccccctcccacccctccccgcagaccccctgtggcccctcccacccaccccagagtgatccccgaagtgtcccacgtgagaggcgtgaccgtgcacatggaaacccccgaagccatcctgttcgcccccggcgaaaccttcggcaccaacgtgtccatccacgccatcgcccacgatgatggcccctacgccatggatgtggtgtggatgagattcgatgtgccctcctcctgcgccgaaatgagaatctacgaagcctgcctgtaccacccccagctgcccgaatgcctgtcccccgccgatgccccctgcgccgtgtcctcctgggcctacagactggccgtgagatcctacgccggctgctccagaaccacccctccccctagatgcttcgccgaagccagaatggaacccgtgcccggcctggcctggctggcctccaccgtgaacctggaattccagcacgccagcccccagcacgccggcctgtacctgtgcgtggtgtacgtggatgatcacatccacgcctggggccacatgaccatctccaccgccgcccagtacagaaacgccgtggtggaacagcacctgccccagagacagcccgaacccgtggaacccaccagaccccacgtgagagcctga

SEQ ID NO: 103 - DNA, 1218 bp, synthetic construct
  source 1..1218: mol_type = other DNA
atggcacggggagccggattggtgttctttgtgggcgtgtgggtggtgagctgcttggcagccgcaccacgcacctcttggaaacgcgttacttccggggaggacgttgtcctccttccagcacccgcaggacctgaggaaaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaggaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 104 - RNA, 1260 nt, synthetic construct
  source 1..1260: mol_type = other RNA
atgcgcatgcagctgctgctgctgatcgccctgtccctggccctggtgaccaactccgcctcccccggccgcaccatcaccgtgggcccccgcggcaacgcctccaacgccgccccctccgcctccccccgcaacgcctccgccccccgcaccacccccacccccccccagccccgcaaggccaccaagtccaaggcctccaccgccaagcccgcccccccccccaagaccggcccccccaagacctcctccgagcccgtgcgctgcaaccgccacgaccccctggcccgctacggctcccgcgtgcagatccgctgccgcttccccaactccacccgcaccgagttccgcctgcagatctggcgctacgccaccgccaccgacgccgagatcggcaccgccccctccctggaggaggtgatggtgaacgtgtccgccccccccggcggccagctggtgtacgactccgcccccaaccgcaccgacccccacgtgatctgggccgagggcgccggccccggcgcctccccccgcctgtactccgtggtgggccccctgggccgccagcgcctgatcatcgaggagctgaccctggagacccagggcatgtactactgggtgtggggccgcaccgaccgcccctccgcctacggcacctgggtgcgcgtgcgcgtgttccgccccccctccctgaccatccacccccacgccgtgctggagggccagcccttcaaggccacctgcaccgccgccacctactaccccggcaaccgcgccgagttcgtgtggttcgaggacggccgccgcgtgttcgaccccgcccagatccacacccagacccaggagaaccccgacggcttctccaccgtgtccaccgtgacctccgccgccgtgggcggccagggccccccccgcaccttcacctgccagctgacctggcaccgcgactccgtgtccttctcccgccgcaacgcctccggcaccgcctccgtgctgccccgccccaccatcaccatggagttcaccggcgaccacgccgtgtgcaccgccggctgcgtgcccgagggcgtgaccttcgcctggttcctgggcgacgactcctcccccgccgagaaggtggccgtggcctcccagacctcctgcggccgccccggcaccgccaccatccgctccaccctgcccgtgtcctacgagcagaccgagtacatctgccgcctggccggctaccccgacggcatccccgtgctggagcaccactaa

SEQ ID NO: 105 - RNA, 1260 nt, synthetic construct
  source 1..1260: mol_type = other RNA
atgagaatgcagctgctgctcctgatcgccctgtctctggccctggtcaccaatagcgcttctcccggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggcccaggcgcaagccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactga

SEQ ID NO: 106 - RNA, 1260 nt, synthetic construct
  source 1..1260: mol_type = other RNA
atgcgcatgcaactgctcctgctgattgcgttgagccttgccctggtgaccaacagcgcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 107 - RNA, 1260 nt, synthetic construct
  source 1..1260: mol_type = other RNA
atgagaatgcagctgctgctgctgatcgccctgtccctggccctggtgaccaactccgcctcccccggcagaaccatcaccgtgggccccagaggcaacgcctccaacgccgccccctccgcctcccccagaaacgcctccgcccccagaaccacccccacccctccccagcccagaaaagccaccaaatccaaagcctccaccgccaaacccgcccctcctcccaaaaccggccctcccaaaacctcctccgaacccgtgagatgcaacagacacgatcccctggccagatacggctccagagtgcagatcagatgcagattccccaactccaccagaaccgaattcagactgcagatctggagatacgccaccgccaccgatgccgaaatcggcaccgccccctccctggaagaagtgatggtgaacgtgtccgcccctcctggcggccagctggtgtacgattccgcccccaacagaaccgatccccacgtgatctgggccgaaggcgccggccccggcgcctcccccagactgtactccgtggtgggccccctgggcagacagagactgatcatcgaagaactgaccctggaaacccagggcatgtactactgggtgtggggcagaaccgatagaccctccgcctacggcacctgggtgagagtgagagtgttcagacccccttccctgaccatccacccccacgccgtgctggaaggccagcccttcaaagccacctgcaccgccgccacctactaccccggcaacagagccgaattcgtgtggttcgaagatggcagaagggtgttcgatcccgcccagatccacacccagacccaggaaaaccccgacggcttctccaccgtgtccaccgtgacctccgccgccgtgggcggccagggccctcccagaaccttcacctgccagctgacctggcacagagactccgtgtccttctccagaagaaacgcctccggcaccgcctccgtgctgcccagacccaccatcaccatggaattcaccggcgatcacgccgtgtgcaccgccggctgcgtgcccgaaggcgtgaccttcgcctggttcctgggcgatgattcctcccccgccgaaaaagtggccgtggcctcccagacctcctgcggcagacccggcaccgccaccatcagatccaccctgcccgtgtcctacgaacagaccgaatacatctgcagactggccggctaccccgatggcatccccgtgctggaacaccactga

SEQ ID NO: 108 - RNA, 1287 nt, synthetic construct
  source 1..1287: mol_type = other RNA
atgggccgcctgacctccggcgtgggcaccgccgccctgctggtggtggccgtgggcctgcgcgtggtgtgcgccaagtacgccgcctcccccggccgcaccatcaccgtgggcccccgcggcaacgcctccaacgccgccccctccgcctccccccgcaacgcctccgccccccgcaccacccccacccccccccagccccgcaaggccaccaagtccaaggcctccaccgccaagcccgcccccccccccaagaccggcccccccaagacctcctccgagcccgtgcgctgcaaccgccacgaccccctggcccgctacggctcccgcgtgcagatccgctgccgcttccccaactccacccgcaccgagttccgcctgcagatctggcgctacgccaccgccaccgacgccgagatcggcaccgccccctccctggaggaggtgatggtgaacgtgtccgccccccccggcggccagctggtgtacgactccgcccccaaccgcaccgacccccacgtgatctgggccgagggcgccggccccggcgcctccccccgcctgtactccgtggtgggccccctgggccgccagcgcctgatcatcgaggagctgaccctggagacccagggcatgtactactgggtgtggggccgcaccgaccgcccctccgcctacggcacctgggtgcgcgtgcgcgtgttccgccccccctccctgaccatccacccccacgccgtgctggagggccagcccttcaaggccacctgcaccgccgccacctactaccccggcaaccgcgccgagttcgtgtggttcgaggacggccgccgcgtgttcgaccccgcccagatccacacccagacccaggagaaccccgacggcttctccaccgtgtccaccgtgacctccgccgccgtgggcggccagggccccccccgcaccttcacctgccagctgacctggcaccgcgactccgtgtccttctcccgccgcaacgcctccggcaccgcctccgtgctgccccgccccaccatcaccatggagttcaccggcgaccacgccgtgtgcaccgccggctgcgtgcccgagggcgtgaccttcgcctggttcctgggcgacgactcctcccccgccgagaaggtggccgtggcctcccagacctcctgcggccgccccggcaccgccaccatccgctccaccctgcccgtgtcctacgagcagaccgagtacatctgccgcctggccggctaccccgacggcatccccgtgctggagcaccactaa

SEQ ID NO: 109 - RNA, 1287 nt, synthetic construct
  source 1..1287: mol_type = other RNA
atggggagactcacatcaggcgtaggaaccgctgccctgttggtcgtggccgttggtctgagagttgtgtgtgccaaatatgctgcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 110 - RNA, 1287 nt, synthetic construct
  source 1..1287: mol_type = other RNA
atggggagactcacatcaggcgtaggaaccgctgccctgttggtcgtggccgttggtctgagagttgtgtgtgccaaatatgctgcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 111 - RNA, 84 nt, synthetic construct
  source 1..84: mol_type = other RNA
atgggcagactgacatctggcgtgggaacagctgctctgctggtggttgctgtgggcctgagagtcgtgtgtgccaaatacgcc

SEQ ID NO: 112 - RNA, 1290 nt, synthetic construct
  source 1..1290: mol_type = other RNA
atgggcagactgacatctggcgtgggaacagctgctctgctggtggttgctgtgggcctgagagtcgtgtgtgccaaatacgccctggcctctcccggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggcccaggcgcaagccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactga

SEQ ID NO: 113 - RNA, 1290 nt, synthetic construct
  source 1..1290: mol_type = other RNA
atggggagactcacatcaggcgtaggaaccgctgccctgttggtcgtggccgttggtctgagagttgtgtgtgccaaatatgctctggcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 114 - RNA, 1290 nt, synthetic construct
  source 1..1290: mol_type = other RNA
atgggcagactgacctccggcgtgggcaccgccgccctgctggtggtggccgtgggcctgagagtggtgtgcgccaaatacgccctggcctcccccggcagaaccatcaccgtgggccccagaggcaacgcctccaacgccgccccctccgcctcccccagaaacgcctccgcccccagaaccacccccacccctccccagcccagaaaagccaccaaatccaaagcctccaccgccaaacccgcccctcctcccaaaaccggccctcccaaaacctcctccgaacccgtgagatgcaacagacacgatcccctggccagatacggctccagagtgcagatcagatgcagattccccaactccaccagaaccgaattcagactgcagatctggagatacgccaccgccaccgatgccgaaatcggcaccgccccctccctggaagaagtgatggtgaacgtgtccgcccctcctggcggccagctggtgtacgattccgcccccaacagaaccgatccccacgtgatctgggccgaaggcgccggccccggcgcctcccccagactgtactccgtggtgggccccctgggcagacagagactgatcatcgaagaactgaccctggaaacccagggcatgtactactgggtgtggggcagaaccgatagaccctccgcctacggcacctgggtgagagtgagagtgttcagacccccttccctgaccatccacccccacgccgtgctggaaggccagcccttcaaagccacctgcaccgccgccacctactaccccggcaacagagccgaattcgtgtggttcgaagatggcagaagggtgttcgatcccgcccagatccacacccagacccaggaaaaccccgacggcttctccaccgtgtccaccgtgacctccgccgccgtgggcggccagggccctcccagaaccttcacctgccagctgacctggcacagagactccgtgtccttctccagaagaaacgcctccggcaccgcctccgtgctgcccagacccaccatcaccatggaattcaccggcgatcacgccgtgtgcaccgccggctgcgtgcccgaaggcgtgaccttcgcctggttcctgggcgatgattcctcccccgccgaaaaagtggccgtggcctcccagacctcctgcggcagacccggcaccgccaccatcagatccaccctgcccgtgtcctacgaacagaccgaatacatctgcagactggccggctaccccgatggcatccccgtgctggaacaccactga

SEQ ID NO: 115 - RNA, 1281 nt, synthetic construct
  source 1..1281: mol_type = other RNA
atggccttggggagagtgggccttgcagtgggtctgtggggactgctctgggttggcgtagtcgtcgtgctggctaacgcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 116 - RNA, 996 nt, synthetic construct
  source 1..996: mol_type = other RNA
atgggccgcctgacctccggcgtgggcaccgccgccctgctggtggtggccgtgggcctgcgcgtggtgtgcgccaagtacgccctggccgacccctccctgaagatggccgaccccaaccgcttccgcggcaagaacctgcccgtgctggaccagctgaccgacccccccggcgtgaagcgcgtgtaccacatccagccctccctggaggaccccttccagcccccctccatccccatcaccgtgtactacgccgtgctggagcgcgcctgccgctccgtgctgctgcacgccccctccgaggccccccagatcgtgcgcggcgcctccgacgaggcccgcaagcacacctacaacctgaccatcgcctggtaccgcatgggcgacaactgcgccatccccatcaccgtgatggagtacaccgagtgcccctacaacaagtccctgggcgtgtgccccatccgcacccagccccgctggtcctactacgactccttctccgccgtgtccgaggacaacctgggcttcctgatgcacgcccccgccttcgagaccgccggcacctacctgcgcctggtgaagatcaacgactggaccgagatcacccagttcatcctggagcaccgcgcccgcgcctcctgcaagtacgccctgcccctgcgcatcccccccgccgcctgcctgacctccaaggcctaccagcagggcgtgaccgtggactccatcggcatgctgccccgcttcatccccgagaaccagcgcaccgtggccctgtactccctgaagatcgccggctggcacggccccaagcccccctacacctccaccctgctgccccccgagctgtccgacaccaccaacgccacccagcccgagctggtgcccgaggaccccgaggactccgccctgctggaggaccccgccggcaccgtgtcctcccagatcccccccaactggcacatcccctccatccaggacgtggccccccaccactaa

SEQ ID NO: 117 - RNA, 996 nt, synthetic construct
  source 1..996: mol_type = other RNA
atgggcagactgacatctggcgtgggaacagctgctctgctggtggttgctgtgggcctgagagtcgtgtgtgccaaatacgccctggccgatcctagcctgaagatggctgaccccaaccggttccggggcaagaatctgcctgttctggaccagctgaccgatcctcctggcgtgaaacgggtgtaccacatccagccaagcctggaagatcccttccagcctcctagcatccccatcaccgtgtactacgccgtgctggaaagggcctgtagaagcgtgctgctgcacgccccatctgaagcccctcaaatcgtcagaggcgcttccgacgaggccagaaagcacacctacaacctgacaatcgcctggtacagaatgggcgacaactgcgccattcctatcaccgtgatggagtacaccgagtgtccctacaacaagagcctgggcgtgtgccccatcagaacacagcctagatggtcctactacgacagcttcagcgccgtgtccgaggacaatctgggcttcctgatgcatgcccctgcctttgagacagccggcacctatctgcggctggtcaagatcaacgactggaccgagatcacccagttcatcctggaacacagagccagagccagctgcaaatacgctctgcccctgagaattcctcctgccgcctgtctgacaagcaaggcctatcagcagggcgtgaccgtggatagcatcggcatgctgcccagattcatccccgagaaccagagaacagtggccctgtactccctgaagatcgccggatggcacggacccaagcctccatacacaagcacactgctgcctccagagctgagcgacaccaccaatgccacacagcctgaactggtgcctgaggacccagaggattctgccctgcttgaagatcctgccggcaccgtgtctagccagattcctcctaactggcacatccccagcatccaggatgtggcccctcatcattga

SEQ ID NO: 118 - RNA, 996 nt, synthetic construct
  source 1..996: mol_type = other RNA
atggggagactcacatcaggcgtaggaaccgctgccctgttggtcgtggccgttggtctgagagttgtgtgtgccaaatatgctctcgctgatccgagcctcaagatggcagatcccaaccgatttcggggaaagaatctgccagtactggaccagctgacggacccacctggcgtcaaacgcgtctaccacatacagcctagtcttgaggacccttttcagccaccgtctatccccattaccgtgtactatgccgtgctggaacgcgcgtgtaggtcagttctgctgcatgccccatccgaagccccccagatcgtcagaggagcttctgatgaagcacgcaaacacacctacaacctcacaatagcgtggtatcgaatgggcgataactgcgcaattcccatcacagtcatggagtacacggagtgcccctacaacaagagcctcggtgtttgccctatcaggacacaacccaggtggagctattacgacagtttcagcgccgtgtctgaggacaatctggggtttctgatgcacgcacccgccttcgagactgccggcacctacttgcggctggtgaagatcaacgactggactgagatcacccagttcatcctggaacatagggccagagccagctgcaagtatgctcttcccctgcggattccgcctgcagcatgtctgacctcaaaagcctaccagcaaggggtgactgtggacagcattggcatgctgcctcgtttcattcccgagaatcaacggacagtggctctgtattccctgaagatcgcaggatggcatgggcccaaaccaccttatacctctacgttgcttccaccagaactcagtgacaccactaatgcgacacagccagaacttgtgcctgaggatcctgaagatagcgctctgttggaggatccagccggtactgtgtcctcccagataccacccaattggcacattccttccattcaggacgtagctccgcatcactga

SEQ ID NO: 119 - RNA, 996 nt, synthetic construct
  source 1..996: mol_type = other RNA
atggggagactcacatcaggcgtaggaaccgctgccctgttggtcgtggccgttggtctgagagttgtgtgtgccaaatatgctctcgctgatccgagcctcaagatggcagatcccaaccgatttcggggaaagaatctgccagtactggaccagctgacggacccacctggcgtcaaacgcgtctaccacatacagcctagtcttgaggacccttttcagccaccgtctatccccattaccgtgtactatgccgtgctggaacgcgcgtgtaggtcagttctgctgcatgccccatccgaagccccccagatcgtcagaggagcttctgatgaagcacgcaaacacacctacaacctcacaatagcgtggtatcgaatgggcgataactgcgcaattcccatcacagtcatggagtacacggagtgcccctacaacaagagcctcggtgtttgccctatcaggacacaacccaggtggagctattacgacagtttcagcgccgtgtctgaggacaatctggggtttctgatgcacgcacccgccttcgagactgccggcacctacttgcggctggtgaagatcaacgactggactgagatcacccagttcatcctggaacatagggccagagccagctgcaagtatgcccttcccctgcggattccgcctgcagcatgtctgacctcaaaagcctaccagcaaggggtgactgtggacagcattggcatgctgcctcgtttcattcccgagaatcaacggacagtggctctgtattccctgaagatcgcaggatggcatgggcccaaaccaccttatacctctacgttgcttccaccagaactcagtgacaccactaatgcgacacagccagaacttgtgcctgaggatcctgaagatagcgctctgttggaggatccagccggtactgtgtcctcccagataccacccaattggcacattccttccattcaggacgtagctccgcatcactga

SEQ ID NO: 120 - RNA, 996 nt, synthetic construct
  source 1..996: mol_type = other RNA
atgggcagactgacctccggcgtgggcaccgccgccctgctggtggtggccgtgggcctgagagtggtgtgcgccaaatacgccctggccgatccctccctgaaaatggccgatcccaacaggttcagaggcaaaaacctgcccgtgctggatcagctgaccgatccccctggcgtgaaaagagtgtaccacatccagccctccctggaagatcccttccagcccccttccatccccatcaccgtgtactacgccgtgctggaaagagcttgcagatccgtgctgctgcacgccccctccgaagcccctcagatcgtgagaggcgcctccgatgaagccagaaaacacacctacaacctgaccatcgcctggtacagaatgggcgataactgcgccatccccatcaccgtgatggaatacaccgaatgcccctacaacaaatccctgggcgtgtgccccatcagaacccagcccagatggtcctactacgattccttctccgccgtgtccgaagataacctgggcttcctgatgcacgcccccgccttcgaaaccgccggcacctacctgagactggtgaaaatcaacgattggaccgaaatcacccagttcatcctggaacacagagccagagcctcctgcaaatacgccctgcccctgagaatccctcccgccgcctgcctgacctccaaagcctaccagcagggcgtgaccgtggattccatcggcatgctgcccagattcatccccgaaaaccagagaaccgtggccctgtactccctgaaaatcgccggctggcacggccccaaacccccttacacctccaccctgctgccccctgaactgtccgataccaccaacgccacccagcccgaactggtgcccgaagatcccgaagattccgccctgctggaagatcccgccggcaccgtgtcctcccagatccctcccaactggcacatcccctccatccaggatgtggcccctcaccactga

SEQ ID NO: 121 - RNA, 1206 nt, synthetic construct
  source 1..1206: mol_type = other RNA
atgcgcatgcagctgctgctgctgatcgccctgtccctggccctggtgaccaactcccgcacctcctggaagcgcgtgacctccggcgaggacgtggtgctgctgcccgcccccgccggccccgaggagcgcacccgcgcccacaagctgctgtgggccgccgagcccctggacgcctgcggccccctgcgcccctcctgggtggccctgtggcccccccgccgcgtgctggagaccgtggtggacgccgcctgcatgcgcgcccccgagcccctggccatcgcctactccccccccttccccgccggcgacgagggcctgtactccgagctggcctggcgcgaccgcgtggccgtggtgaacgagtccctggtgatctacggcgccctggagaccgactccggcctgtacaccctgtccgtggtgggcctgtccgacgaggcccgccaggtggcctccgtggtgctggtggtggagcccgcccccgtgcccacccccacccccgacgactacgacgaggaggacgacgccggcgtgtccgagcgcacccccgtgtccgtgcccccccccacccccccccgccgcccccccgtggccccccccacccacccccgcgtgatccccgaggtgtcccacgtgcgcggcgtgaccgtgcacatggagacccccgaggccatcctgttcgcccccggcgagaccttcggcaccaacgtgtccatccacgccatcgcccacgacgacggcccctacgccatggacgtggtgtggatgcgcttcgacgtgccctcctcctgcgccgagatgcgcatctacgaggcctgcctgtaccacccccagctgcccgagtgcctgtcccccgccgacgccccctgcgccgtgtcctcctgggcctaccgcctggccgtgcgctcctacgccggctgctcccgcaccaccccccccccccgctgcttcgccgaggcccgcatggagcccgtgcccggcctggcctggctggcctccaccgtgaacctggagttccagcacgcctccccccagcacgccggcctgtacctgtgcgtggtgtacgtggacgaccacatccacgcctggggccacatgaccatctccaccgccgcccagtaccgcaacgccgtggtggagcagcacctgccccagcgccagcccgagcccgtggagcccacccgcccccacgtgcgcgcctaa

SEQ ID NO: 122 - RNA, 1206 nt, synthetic construct
  source 1..1206: mol_type = other RNA
atgagaatgcagctgctgctcctgatcgccctgtctctggccctggtcaccaatagcagaaccagctggaaaagagtgaccagcggcgaggatgtggtgctgcttcctgctcctgctggccccgaggaaagaacaagagcccacaaactgctgtgggccgctgagcctcttgatgcctgtggacctctcagacctagctgggttgcactgtggccacctcggagagtgctggaaacagtggtggatgccgcctgcatgagagcccctgaacctctggccattgcctactctccaccatttccagccggcgacgagggcctgtattctgagcttgcttggagagacagagtggccgtggtcaacgagagcctggttatctatggcgccctggaaaccgacagcggcctgtacacactgtctgtcgtgggcctgtctgacgaggctagacaggtggcatctgtggtcctggtggtggaacctgctccagtgcctacacctacacctgacgactacgacgaggaagatgacgctggcgtcagcgagagaacccctgtttctgtgcctcctcctacgcctcctcgtagacctcctgttgctcctccaacacaccccagagtgatccctgaagtgtctcacgtgcggggcgtgaccgtgcacatggaaacacctgaggccatcctgttcgcccctggcgagacatttggcaccaacgtgtccatccacgctatcgcccacgacgatggcccttacgccatggatgtcgtgtggatgagattcgacgtgcccagcagctgtgccgagatgagaatctatgaggcctgcctgtatcaccctcagctgcccgaatgtctgagccctgctgatgccccttgtgccgttagcagctgggcctatagactggccgtgcggtcttatgccggctgctctagaacaacccctcctcctcggtgtttcgccgaggccagaatggaacctgttcctggactggcctggctggcctccacagtgaacctggaatttcagcacgcctctccacagcacgccggcctgtatctgtgtgtggtgtacgtggacgatcacatccacgcctggggccacatgaccatctctacagccgctcagtaccggaacgccgtggttgaacagcatctgcctcagagacagcccgagcctgtggaacctacaagacctcatgttcgggcctga

SEQ ID NO: 123 - RNA, 1206 nt, synthetic construct
  source 1..1206: mol_type = other RNA
atgcgaatgcagcttctgctgctcattgccttgtcccttgccttggtgaccaactcacgcacctcttggaaacgcgttacttccggggaggacgttgtcctccttccagcacccgcaggacctgaagagaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaagaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 124 - RNA, 1206 nt, synthetic construct
  source 1..1206: mol_type = other RNA
atgagaatgcagctgctgctgctgatcgccctgtccctggccctggtgaccaactccagaacctcctggaaaagagtgacctccggcgaagatgtggtgctgctgcccgcccccgccggccccgaagaaagaaccagagcccacaaactgctgtgggccgccgaacccctggatgcctgcggccccctcagaccctcctgggtggccctgtggccccctagaagggtgctggaaaccgtggtggatgccgcctgcatgagagcccccgaacccctggccatcgcctactcccctcccttccccgccggcgatgaaggcctgtactccgaactggcctggagagatagagtggccgtggtgaacgaatccctggtgatctacggcgccctggaaaccgattccggcctgtacaccctgtccgtggtgggcctgtccgatgaagccagacaggtggcctccgtggtgctggtggtggaacccgcccccgtgcccacccccacccccgatgattacgatgaagaagatgatgccggcgtgtccgaaagaacccccgtgtccgtgccccctcccacccctccccgcagaccccctgtggcccctcccacccaccccagagtgatccccgaagtgtcccacgtgagaggcgtgaccgtgcacatggaaacccccgaagccatcctgttcgcccccggcgaaaccttcggcaccaacgtgtccatccacgccatcgcccacgatgatggcccctacgccatggatgtggtgtggatgagattcgatgtgccctcctcctgcgccgaaatgagaatctacgaagcctgcctgtaccacccccagctgcccgaatgcctgtcccccgccgatgccccctgcgccgtgtcctcctgggcctacagactggccgtgagatcctacgccggctgctccagaaccacccctccccctagatgcttcgccgaagccagaatggaacccgtgcccggcctggcctggctggcctccaccgtgaacctggaattccagcacgccagcccccagcacgccggcctgtacctgtgcgtggtgtacgtggatgatcacatccacgcctggggccacatgaccatctccaccgccgcccagtacagaaacgccgtggtggaacagcacctgccccagagacagcccgaacccgtggaacccaccagaccccacgtgagagcctga

SEQ ID NO: 125 - RNA, 1233 nt, synthetic construct
  source 1..1233: mol_type = other RNA
atgggccgcctgacctccggcgtgggcaccgccgccctgctggtggtggccgtgggcctgcgcgtggtgtgcgccaagtacgcccgcacctcctggaagcgcgtgacctccggcgaggacgtggtgctgctgcccgcccccgccggccccgaggagcgcacccgcgcccacaagctgctgtgggccgccgagcccctggacgcctgcggccccctgcgcccctcctgggtggccctgtggcccccccgccgcgtgctggagaccgtggtggacgccgcctgcatgcgcgcccccgagcccctggccatcgcctactccccccccttccccgccggcgacgagggcctgtactccgagctggcctggcgcgaccgcgtggccgtggtgaacgagtccctggtgatctacggcgccctggagaccgactccggcctgtacaccctgtccgtggtgggcctgtccgacgaggcccgccaggtggcctccgtggtgctggtggtggagcccgcccccgtgcccacccccacccccgacgactacgacgaggaggacgacgccggcgtgtccgagcgcacccccgtgtccgtgcccccccccacccccccccgccgcccccccgtggccccccccacccacccccgcgtgatccccgaggtgtcccacgtgcgcggcgtgaccgtgcacatggagacccccgaggccatcctgttcgcccccggcgagaccttcggcaccaacgtgtccatccacgccatcgcccacgacgacggcccctacgccatggacgtggtgtggatgcgcttcgacgtgccctcctcctgcgccgagatgcgcatctacgaggcctgcctgtaccacccccagctgcccgagtgcctgtcccccgccgacgccccctgcgccgtgtcctcctgggcctaccgcctggccgtgcgctcctacgccggctgctcccgcaccaccccccccccccgctgcttcgccgaggcccgcatggagcccgtgcccggcctggcctggctggcctccaccgtgaacctggagttccagcacgcctccccccagcacgccggcctgtacctgtgcgtggtgtacgtggacgaccacatccacgcctggggccacatgaccatctccaccgccgcccagtaccgcaacgccgtggtggagcagcacctgccccagcgccagcccgagcccgtggagcccacccgcccccacgtgcgcgcctaa

SEQ ID NO: 126 - RNA, 1233 nt, synthetic construct
  source 1..1233: mol_type = other RNA
atgggcagactgacatctggcgtgggaacagctgctctgctggtggttgctgtgggcctgagagtcgtgtgtgccaaatacgccagaaccagctggaaaagagtgaccagcggcgaggatgtggtgctgcttcctgctcctgctggccccgaggaaagaacaagagcccacaaactgctgtgggccgctgagcctcttgatgcctgtggacctctcagacctagctgggttgcactgtggccacctcggagagtgctggaaacagtggtggatgccgcctgcatgagagcccctgaacctctggccattgcctactctccaccatttccagccggcgacgagggcctgtattctgagcttgcttggagagacagagtggccgtggtcaacgagagcctggttatctatggcgccctggaaaccgacagcggcctgtacacactgtctgtcgtgggcctgtctgacgaggctagacaggtggcatctgtggtcctggtggtggaacctgctccagtgcctacacctacacctgacgactacgacgaggaagatgacgctggcgtcagcgagagaacccctgtttctgtgcctcctcctacgcctcctcgtagacctcctgttgctcctccaacacaccccagagtgatccctgaagtgtctcacgtgcggggcgtgaccgtgcacatggaaacacctgaggccatcctgttcgcccctggcgagacatttggcaccaacgtgtccatccacgctatcgcccacgacgatggcccttacgccatggatgtcgtgtggatgagattcgacgtgcccagcagctgtgccgagatgagaatctatgaggcctgcctgtatcaccctcagctgcccgaatgtctgagccctgctgatgccccttgtgccgttagcagctgggcctatagactggccgtgcggtcttatgccggctgctctagaacaacccctcctcctcggtgtttcgccgaggccagaatggaacctgttcctggactggcctggctggcctccacagtgaacctggaatttcagcacgcctctccacagcacgccggcctgtatctgtgtgtggtgtacgtggacgatcacatccacgcctggggccacatgaccatctctacagccgctcagtaccggaacgccgtggttgaacagcatctgcctcagagacagcccgagcctgtggaacctacaagacctcatgttcgggcctga

SEQ ID NO: 127 - RNA, 1233 nt, synthetic construct
  source 1..1233: mol_type = other RNA
atggggagactcacatcaggcgtaggaaccgctgccctgttggtcgtggccgttggtctgagagttgtgtgtgccaaatatgctcgcacctcttggaaacgcgttacttccggggaggacgttgtcctccttccagcacccgcaggacctgaggaaaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaggaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 128 - RNA, 1233 nt, synthetic construct
  source 1..1233: mol_type = other RNA
atggggagactcacatcaggcgtaggaaccgctgccctgttggtcgtggccgttggtctgagagttgtgtgtgccaaatatgctcgcacctcttggaaacgcgttacttccggggaggacgttgtcctccttccagcacccgcaggacctgaagagaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaagaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 129 - RNA, 1233 nt, synthetic construct
  source 1..1233: mol_type = other RNA
atgggcagactgacctccggcgtgggcaccgccgccctgctggtggtggccgtgggcctgagagtggtgtgcgccaaatacgccagaacctcctggaaaagagtgacctccggcgaagatgtggtgctgctgcccgcccccgccggccccgaagaaagaaccagagcccacaaactgctgtgggccgccgaacccctggatgcctgcggccccctcagaccctcctgggtggccctgtggccccctagaagggtgctggaaaccgtggtggatgccgcctgcatgagagcccccgaacccctggccatcgcctactcccctcccttccccgccggcgatgaaggcctgtactccgaactggcctggagagatagagtggccgtggtgaacgaatccctggtgatctacggcgccctggaaaccgattccggcctgtacaccctgtccgtggtgggcctgtccgatgaagccagacaggtggcctccgtggtgctggtggtggaacccgcccccgtgcccacccccacccccgatgattacgatgaagaagatgatgccggcgtgtccgaaagaacccccgtgtccgtgccccctcccacccctccccgcagaccccctgtggcccctcccacccaccccagagtgatccccgaagtgtcccacgtgagaggcgtgaccgtgcacatggaaacccccgaagccatcctgttcgcccccggcgaaaccttcggcaccaacgtgtccatccacgccatcgcccacgatgatggcccctacgccatggatgtggtgtggatgagattcgatgtgccctcctcctgcgccgaaatgagaatctacgaagcctgcctgtaccacccccagctgcccgaatgcctgtcccccgccgatgccccctgcgccgtgtcctcctgggcctacagactggccgtgagatcctacgccggctgctccagaaccacccctccccctagatgcttcgccgaagccagaatggaacccgtgcccggcctggcctggctggcctccaccgtgaacctggaattccagcacgccagcccccagcacgccggcctgtacctgtgcgtggtgtacgtggatgatcacatccacgcctggggccacatgaccatctccaccgccgcccagtacagaaacgccgtggtggaacagcacctgccccagagacagcccgaacccgtggaacccaccagaccccacgtgagagcctga

SEQ ID NO: 130 - RNA, 1218 nt, synthetic construct
  source 1..1218: mol_type = other RNA
atggcacggggagccggattggtgttctttgtgggcgtgtgggtggtgagctgcttggcagccgcaccacgcacctcttggaaacgcgttacttccggggaggacgttgtcctccttccagcacccgcaggacctgaggaaaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaggaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 131 - protein, 426 aa, synthetic construct
MALGRVGLAVGLWGLLWVGVVVVLANASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 132 - protein, 405 aa, synthetic construct
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRA

SEQ ID NO: 133 - DNA, 18 bp, synthetic construct
  source 1..18: mol_type = other DNA
ccatgcgcacgaggaagt

SEQ ID NO: 134 - DNA, 13 bp, synthetic construct
  source 1..13: mol_type = other DNA
  modified_base 1: mod_base = OTHER; note = 5' 6-fluorescein amidite modified nucleotide
  modified_base 13: mod_base = OTHER; note = 3' minor groove binder nonfluorescent quencher modified nucleotide
cgaggccgccaac

SEQ ID NO: 135 - DNA, 21 bp, synthetic construct
  source 1..21: mol_type = other DNA
catgacaacttcggcattgtg

SEQ ID NO: 136 - DNA, 20 bp, synthetic construct
  source 1..20: mol_type = other DNA
tcttctgggtggcagtgatg

SEQ ID NO: 137 - DNA, 19 bp, synthetic construct
  source 1..19: mol_type = other DNA
  misc_feature 1: note = Fluorescent reporter VIC
  modified_base 19: mod_base = OTHER; note = 3' minor groove binder nonfluorescent quencher modified nucleotide
agggactcatgaccacagt

SEQ ID NO: 138 - DNA, 81 bp, synthetic construct
  source 1..81: mol_type = other DNA
atggccttggggagagtgggccttgcagtgggtctgtggggactgctctgggttggcgtagtcgtcgtgctggctaacgca

SEQ ID NO: 139 - DNA, 69 bp, synthetic construct
  source 1..69: mol_type = other DNA
atggcacggggagccggattggtgttctttgtgggcgtgtgggtggtgagctgcttggcagccgcacca

SEQ ID NO: 140 - RNA, 81 nt, synthetic construct
  source 1..81: mol_type = other RNA
atggccttggggagagtgggccttgcagtgggtctgtggggactgctctgggttggcgtagtcgtcgtgctggctaacgca

SEQ ID NO: 141 - RNA, 69 nt, synthetic construct
  source 1..69: mol_type = other RNA
atggcacggggagccggattggtgttctttgtgggcgtgtgggtggtgagctgcttggcagccgcacca

SEQ ID NO: 142 - DNA, 906 bp, synthetic construct
  source 1..906: mol_type = other DNA
gatccgagcctcaagatggcagatcccaaccgatttcggggaaagaatctgccagtactggaccagctgacggacccacctggcgtcaaacgcgtctaccacatacagcctagtcttgaggacccttttcagccaccgtctatccccattaccgtgtactatgccgtgctggaacgcgcgtgtaggtcagttctgctgcatgccccatccgaagccccccagatcgtcagaggagcttctgatgaagcacgcaaacacacctacaacctcacaatagcgtggtatcgaatgggcgataactgcgcaattcccatcacagtcatggagtacacggagtgcccctacaacaagagcctcggtgtttgccctatcaggacacaacccaggtggagctattacgacagtttcagcgccgtgtctgaggacaatctggggtttctgatgcacgcacccgccttcgagactgccggcacctacttgcggctggtgaagatcaacgactggactgagatcacccagttcatcctggaacatagggccagagccagctgcaagtatgcccttcccctgcggattccgcctgcagcatgtctgacctcaaaagcctaccagcaaggggtgactgtggacagcattggcatgctgcctcgtttcattcccgagaatcaacggacagtggctctgtattccctgaagatcgcaggatggcatgggcccaaaccaccttatacctctacgttgcttccaccagaactcagtgacaccactaatgcgacacagccagaacttgtgcctgaggatcctgaagatagcgctctgttggaggatccagccggtactgtgtcctcccagataccacccaattggcacattccttccattcaggacgtagctccgcatcactga

SEQ ID NO: 143 - RNA, 906 nt, synthetic construct
  source 1..906: mol_type = other RNA
gatccgagcctcaagatggcagatcccaaccgatttcggggaaagaatctgccagtactggaccagctgacggacccacctggcgtcaaacgcgtctaccacatacagcctagtcttgaggacccttttcagccaccgtctatccccattaccgtgtactatgccgtgctggaacgcgcgtgtaggtcagttctgctgcatgccccatccgaagccccccagatcgtcagaggagcttctgatgaagcacgcaaacacacctacaacctcacaatagcgtggtatcgaatgggcgataactgcgcaattcccatcacagtcatggagtacacggagtgcccctacaacaagagcctcggtgtttgccctatcaggacacaacccaggtggagctattacgacagtttcagcgccgtgtctgaggacaatctggggtttctgatgcacgcacccgccttcgagactgccggcacctacttgcggctggtgaagatcaacgactggactgagatcacccagttcatcctggaacatagggccagagccagctgcaagtatgcccttcccctgcggattccgcctgcagcatgtctgacctcaaaagcctaccagcaaggggtgactgtggacagcattggcatgctgcctcgtttcattcccgagaatcaacggacagtggctctgtattccctgaagatcgcaggatggcatgggcccaaaccaccttatacctctacgttgcttccaccagaactcagtgacaccactaatgcgacacagccagaacttgtgcctgaggatcctgaagatagcgctctgttggaggatccagccggtactgtgtcctcccagataccacccaattggcacattccttccattcaggacgtagctccgcatcactga

SEQ ID NO: 144 - DNA, 1203 bp, synthetic construct
  source 1..1203: mol_type = other DNA
gcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 145 - DNA, 921 bp, synthetic construct
  source 1..921: mol_type = other DNA
aaatatgctctcgctgatccgagcctcaagatggcagatcccaaccgatttcggggaaagaatctgccagtactggaccagctgacggacccacctggcgtcaaacgcgtctaccacatacagcctagtcttgaggacccttttcagccaccgtctatccccattaccgtgtactatgccgtgctggaacgcgcgtgtaggtcagttctgctgcatgccccatccgaagccccccagatcgtcagaggagcttctgatgaagcacgcaaacacacctacaacctcacaatagcgtggtatcgaatgggcgataactgcgcaattcccatcacagtcatggagtacacggagtgcccctacaacaagagcctcggtgtttgccctatcaggacacaacccaggtggagctattacgacagtttcagcgccgtgtctgaggacaatctggggtttctgatgcacgcacccgccttcgagactgccggcacctacttgcggctggtgaagatcaacgactggactgagatcacccagttcatcctggaacatagggccagagccagctgcaagtatgcccttcccctgcggattccgcctgcagcatgtctgacctcaaaagcctaccagcaaggggtgactgtggacagcattggcatgctgcctcgtttcattcccgagaatcaacggacagtggctctgtattccctgaagatcgcaggatggcatgggcccaaaccaccttatacctctacgttgcttccaccagaactcagtgacaccactaatgcgacacagccagaacttgtgcctgaggatcctgaagatagcgctctgttggaggatccagccggtactgtgtcctcccagataccacccaattggcacattccttccattcaggacgtagctccgcatcactga

SEQ ID NO: 146 - DNA, 1149 bp, synthetic construct
  source 1..1149: mol_type = other DNA
cgcacctcttggaaacgcgttacttccggggaggacgttgtcctccttccagcacccgcaggacctgaagagaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaagaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 147 - RNA, 1203 nt, synthetic construct
  source 1..1203: mol_type = other RNA
gcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 148 - skipped

SEQ ID NO: 149 - RNA, 1149 nt, synthetic construct
  source 1..1149: mol_type = other RNA
cgcacctcttggaaacgcgttacttccggggaggacgttgtcctccttccagcacccgcaggacctgaagagaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaagaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 150 - skipped

SEQ ID NO: 151 - DNA, 53 bp, synthetic construct
  source 1..53: mol_type = other DNA
agaataaactagtattcttctggtccccacagactcagagagaacccgccacc

SEQ ID NO: 152 - RNA, 47 nt, synthetic construct
  source 1..47: mol_type = other RNA
aactagtattcttctggtccccacagactcagagagaacccgccacc

SEQ ID NO: 153 - DNA, 110 bp, synthetic construct
  source 1..110: mol_type = other DNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 154 - skipped

SEQ ID NO: 155 - RNA, 110 nt, synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 156 - skipped

SEQ ID NO: 157 - DNA, 278 bp, synthetic construct
  source 1..278: mol_type = other DNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 158 - RNA, 278 nt, synthetic construct
  source 1..278: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 159 - protein, 426 aa, synthetic construct
MGGAAARLGAVILFVVIVGLHGVRGKYSPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 160 - protein, 431 aa, synthetic construct
MHQGAPSWGRRWFVVWALLGLTLGVLVASAAPSPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 161 - protein, 420 aa, synthetic construct
MPGRSLQGLAILGLWVCATGLSPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 162 - protein, 425 aa, synthetic construct
MARGAGLVFFVGVWVVSCLAAAPRTSSPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 163 - protein, 433 aa, synthetic construct
MGVTGILQLPRDRFKRTSFFLWVIILFQRTFSIPSPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 164 - protein, 409 aa, synthetic construct
MGGAAARLGAVILFVVIVGLHGVRGKYRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRA

SEQ ID NO: 165 - DNA, 1284 bp, synthetic construct
  source 1..1284: mol_type = other DNA
atgggcggagctgctgctagactgggagccgtgatcctgttcgtggttatcgtgggactgcatggcgtgcggggcaagtatagccctggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggccctggcgcttcccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactgataa

SEQ ID NO: 166 - DNA, 1284 bp, synthetic construct
  source 1..1284: mol_type = other DNA
atgggaggagcagctgccagactcggtgccgtgatcctgttcgtggtcattgttggcctgcacggggtaaggggcaagtacagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 167 - DNA, 1284 bp, synthetic construct
  source 1..1284: mol_type = other DNA
atgggaggagctgctgctagattgggagctgtgattctgtttgtggtgattgtgggactgcatggagtgagaggaaaatactctcctggaagaaccatcacagtgggaccaagaggaaatgcaagcaatgcagcaccttctgcttctccaagaaatgcttctgctccaagaaccaccccaacccctcctcagccaagaaaagcaaccaaatccaaagcatccacagcaaaacctgcacctcctccaaaaacaggacctccaaaaacctcctctgaacctgtgagatgcaacagacatgatcctctggcaagatatggatcaagagtgcagatcagatgcagatttccaaattccaccagaacagaattcagactccagatctggagatatgcaacagcaacagatgcagaaattggaacagcaccatctctggaagaagtgatggtgaatgtgtctgctcctcctggaggacagctggtgtatgattctgctccaaacagaacagatcctcatgtgatctgggctgaaggagctggacctggagcttctccaagactgtactctgtggtgggacctctgggaagacagagactgatcattgaagaactgaccctggaaacccagggaatgtactactgggtgtggggaagaacagacagaccttctgcttatggaacctgggtgagagtgagagtgttcagacctccttctctgaccatccaccctcatgctgtgctggaaggacagcctttcaaagcaacctgcacagcagcaacctactaccctggaaacagagctgaatttgtgtggtttgaagatggaagaagggtgtttgatcctgctcagatccacacccagacccaggaaaatcctgatggattttccacagtgtccacagtgacatctgctgctgtgggaggacagggacctccaagaaccttcacctgccagctgacctggcacagagattctgtgtctttttcaagaagaaatgcttctggaacagcttctgtgctgccaagaccaaccatcaccatggaattcacaggagatcatgctgtgtgcacagctggatgtgtgcctgaaggagtgacctttgcttggtttctgggagatgattcttctccagctgaaaaagtggctgtggcttcccagacctcttgtggaagacctggaacagcaaccatcagatccaccctgcctgtgtcttatgaacagacagaatacatttgcagactggctggataccctgatggaatccctgtgctggaacaccactgataa

SEQ ID NO: 168 - DNA, 1281 bp, synthetic construct
  source 1..1281: mol_type = other DNA
atgggcggcgcagccgctagactgggtgcagtcatcctctttgtggtgatcgtggggctgcatggggtcagagggaagtattctccgggacggactataaccgtaggtccaagaggaaacgcctctaacgcagccccgtctgcctcaccacgaaacgcctcagctcccagaaccactcctactccaccccagcctaggaaggcgacgaaatccaaggcttccacggccaaacccgcccctccacccaaaaccggacctcctaagaccagctctgaaccggtgcggtgtaataggcacgacccattggcgcgatatggcagtagggtccagatacggtgcagattcccaaacagcacaagaacagaattccggctgcaaatctggcgatatgcaacggccaccgatgccgaaatcggaacagcacccagtctggaagaagtgatggtgaacgtcagtgctccacctggcggacaactggtgtacgactctgcacccaatcgcacagatccccacgtgatttgggccgagggtgctggacctggggcttcacccaggctgtatagcgttgtagggccacttgggaggcagagactcatcattgaggaactgaccctggaaactcagggcatgtactactgggtatggggccgcacagatcgccccagcgcttatggcacctgggtgcgggtgcgggtgtttcgcccaccctccctcaccattcaccctcatgcggttctggagggacagcctttcaaggcaacttgtaccgcagccacctactatcccggcaatagagcggagttcgtctggtttgaggacggccgtagggtgttcgatcctgcccagattcacacccagacacaggagaatcccgacggctttagcacagtgagcactgtgacgtctgctgccgttggtggtcaagggcctcctcgtaccttcacatgccaattgacctggcaccgcgactcagttagctttagccgccggaatgccagtgggaccgccagtgttctcccaaggccgacaatcaccatggagttcactggcgaccatgcagtgtgcacagctgggtgtgtcccagaaggcgtgactttcgcctggtttctgggtgatgactcctcacccgccgagaaagtagctgtcgcttcccagacttcctgtggacgtcctggaactgcgacaatccgaagcacactgccggtttcctacgagcagacggagtacatatgccgccttgcaggctaccccgatggaattccagtccttgagcaccattga

SEQ ID NO: 169 - DNA, 1299 bp, synthetic construct
  source 1..1299: mol_type = other DNA
atgcatcagggcgctccatcttggggtagacgttggttcgttgtgtgggccctgctgggactgacactgggagttctggttgcctctgctgctcctagccctggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggccctggcgcttcccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactgataa

SEQ ID NO: 170 - DNA, 1299 bp, synthetic construct
  source 1..1299: mol_type = other DNA
atgcaccagggtgccccttcctggggcagaaggtggttcgtggtgtgggcccttctggggctgaccctcggagtcctggttgcgagcgcagctcccagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 171 - DNA, 1299 bp, synthetic construct
  source 1..1299: mol_type = other DNA
atgcaccagggagcaccttcttggggaagaagatggtttgtggtgtgggctctgctgggactgaccctgggagtgctggtggcttctgctgctccttctcctggaagaaccatcacagtgggaccaagaggaaatgcaagcaatgcagcaccttctgcttctccaagaaatgcttctgctccaagaaccaccccaacccctcctcagccaagaaaagcaaccaaatccaaagcatccacagcaaaacctgcacctcctccaaaaacaggacctccaaaaacctcctctgaacctgtgagatgcaacagacatgatcctctggcaagatatggatcaagagtgcagatcagatgcagatttccaaattccaccagaacagaattcagactccagatctggagatatgcaacagcaacagatgcagaaattggaacagcaccatctctggaagaagtgatggtgaatgtgtctgctcctcctggaggacagctggtgtatgattctgctccaaacagaacagatcctcatgtgatctgggctgaaggagctggacctggagcttctccaagactgtactctgtggtgggacctctgggaagacagagactgatcattgaagaactgaccctggaaacccagggaatgtactactgggtgtggggaagaacagacagaccttctgcttatggaacctgggtgagagtgagagtgttcagacctccttctctgaccatccaccctcatgctgtgctggaaggacagcctttcaaagcaacctgcacagcagcaacctactaccctggaaacagagctgaatttgtgtggtttgaagatggaagaagggtgtttgatcctgctcagatccacacccagacccaggaaaatcctgatggattttccacagtgtccacagtgacatctgctgctgtgggaggacagggacctccaagaaccttcacctgccagctgacctggcacagagattctgtgtctttttcaagaagaaatgcttctggaacagcttctgtgctgccaagaccaaccatcaccatggaattcacaggagatcatgctgtgtgcacagctggatgtgtgcctgaaggagtgacctttgcttggtttctgggagatgattcttctccagctgaaaaagtggctgtggcttcccagacctcttgtggaagacctggaacagcaaccatcagatccaccctgcctgtgtcttatgaacagacagaatacatttgcagactggctggataccctgatggaatccctgtgctggaacaccactgataa

SEQ ID NO: 172 - DNA, 1296 bp, synthetic construct
  source 1..1296: mol_type = other DNA
atgcatcaaggcgcacccagttggggcagacggtggttcgtagtgtgggccttgctgggactgactctcggtgtcctcgttgcgtctgctgcaccgagtccaggaaggacgattacggtgggacccagaggtaatgcgtccaatgctgcgccatccgcttctccacggaacgcatctgcacccaggactacaccgacaccacctcagccgcgcaaagccaccaagagcaaggccagcacagccaaacccgctcctccacctaaaaccggaccacctaagaccagctctgaacccgtcagatgcaacaggcacgatccgttggccagatatggcagtcgcgtccagatcaggtgtcgcttccctaacagcacacggaccgagttcaggctgcaaatttggcgctacgctacagccactgacgcagagattggcactgctcccagtctggaggaggtcatggtgaacgtgtctgctccaccaggcggtcagctggtctatgactcagcccctaatcgcacagatcctcacgtgatttgggcagaaggtgcggggcctggggcctccccaaggctctactcagtggttggaccccttgggagacagcggctgatcatcgaggaactgactctcgaaacccaaggtatgtactactgggtatggggcagaacagacagaccttcagcttatggcacctgggtgcgggtgagagtgtttaggcctccctccctgacgatccatccccatgctgtgctggaaggacagccgttcaaggcaacatgcacagcagccacttactatcccggaaaccgtgctgagtttgtgtggttcgaggatgggcgacgtgtattcgaccctgcccagattcacacccagacacaggagaatcccgacgggttttccactgtgagcaccgtgacatcagcggcagtaggagggcagggcccaccccgaacgttcacttgccagcttacttggcatcgggacagtgttagctttagccgccggaatgcctctggcaccgcatccgtccttcctcgcccaaccatcaccatggaattcactggcgatcacgccgtttgtacagccgggtgtgttcccgagggagtgacctttgcttggtttctgggcgatgactcaagcccagccgaaaaggtggccgtcgcctcccaaacgagctgtgggcgacctggcaccgctaccatacgtagcactctgcccgtttcctacgaacagaccgagtatatctgccgattggccggttaccccgatgggataccagtcctggagcaccactga

SEQ ID NO: 173 - DNA, 1266 bp, synthetic construct
  source 1..1266: mol_type = other DNA
atgcctggcagatctctgcaaggactggccatcctcggactgtgggtttgcgcaacaggactgagccctggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggccctggcgcttcccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactgataa

SEQ ID NO: 174 - DNA, 1266 bp, synthetic construct
  source 1..1266: mol_type = other DNA
atgcccggcagaagcctccagggactggctatcctggggctgtgggtgtgcgccaccggtcttagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 175 - DNA, 1266 bp, synthetic construct
  source 1..1266: mol_type = other DNA
atgcctggaagatctctccagggactggcaatcctgggactgtgggtgtgtgcaacaggactgtctcctggaagaaccatcacagtgggaccaagaggaaatgcaagcaatgcagcaccttctgcttctccaagaaatgcttctgctccaagaaccaccccaacccctcctcagccaagaaaagcaaccaaatccaaagcatccacagcaaaacctgcacctcctccaaaaacaggacctccaaaaacctcctctgaacctgtgagatgcaacagacatgatcctctggcaagatatggatcaagagtgcagatcagatgcagatttccaaattccaccagaacagaattcagactccagatctggagatatgcaacagcaacagatgcagaaattggaacagcaccatctctggaagaagtgatggtgaatgtgtctgctcctcctggaggacagctggtgtatgattctgctccaaacagaacagatcctcatgtgatctgggctgaaggagctggacctggagcttctccaagactgtactctgtggtgggacctctgggaagacagagactgatcattgaagaactgaccctggaaacccagggaatgtactactgggtgtggggaagaacagacagaccttctgcttatggaacctgggtgagagtgagagtgttcagacctccttctctgaccatccaccctcatgctgtgctggaaggacagcctttcaaagcaacctgcacagcagcaacctactaccctggaaacagagctgaatttgtgtggtttgaagatggaagaagggtgtttgatcctgctcagatccacacccagacccaggaaaatcctgatggattttccacagtgtccacagtgacatctgctgctgtgggaggacagggacctccaagaaccttcacctgccagctgacctggcacagagattctgtgtctttttcaagaagaaatgcttctggaacagcttctgtgctgccaagaccaaccatcaccatggaattcacaggagatcatgctgtgtgcacagctggatgtgtgcctgaaggagtgacctttgcttggtttctgggagatgattcttctccagctgaaaaagtggctgtggcttcccagacctcttgtggaagacctggaacagcaaccatcagatccaccctgcctgtgtcttatgaacagacagaatacatttgcagactggctggataccctgatggaatccctgtgctggaacaccactgataa

SEQ ID NO: 176 - DNA, 1263 bp, synthetic construct
  source 1..1263: mol_type = other DNA
atgcctgggagaagcctgcaagggctcgcaatcttgggcctgtgggtttgtgccacaggcttgtccccgggtcgaacaatcactgttgggcccaggggaaatgccagcaatgctgcaccttcagcaagcccacgaaacgcttcagcacccaggacaacacccactccacctcaaccgcggaaagccaccaagagcaaggcaagtaccgccaaacccgctcctcctcccaagacagggccacccaagacctctagtgagccagtgaggtgtaaccgccatgatccccttgccagatacgggagcagagtgcagattaggtgccggtttccaaactccacgagaaccgaatttcgcctccagatttggcggtatgcgactgccacagacgcagagattggtaccgctcccagcctggaggaggtcatggtgaacgtgtcagcgcctccgggtggccagctggtctacgactctgccccaaatcgaaccgaccctcacgtcatctgggctgaaggagcgggaccaggagcctctccacgcttgtatagcgtagttggccctctggggagacagcgcctgatcattgaggaactgacccttgagacacaggggatgtactactgggtgtggggcaggactgacaggcccagtgcctatggaacttgggttagggtccgcgtctttcggccacccagtctgaccatccatccacatgccgtgctggaaggccagcccttcaaagcgacttgcactgccgccacgtactatccagggaatagagccgagttcgtttggttcgaggatggccggagagtattcgatccagctcagatccacacccagacgcaggaaaacccggacggctttagcacggtgagtaccgtcacctctgctgccgtcggaggccaaggacctccccgtaccttcacatgccagcttacatggcaccgggactcagtaagcttttcacgtcgtaatgcatccggtactgcttctgtgctgcctcgacccaccatcaccatggagttcacaggggatcacgcagtgtgtacggcaggctgcgtgcctgaaggcgtgacattcgcctggtttctcggtgatgactcctctcctgctgaaaaggtggctgtagcctcccaaacaagctgtggtcggcctggaactgccactatacgctccactctcccggtgtcctacgaacagaccgagtacatatgcagactggctggatatcccgatggcattcccgtgctggagcatcactga

SEQ ID NO: 177 - DNA, 1281 bp, synthetic construct
  source 1..1281: mol_type = other DNA
atggctagaggtgccggcctggtgttctttgttggcgtgtgggtcgtgtcctgtctggccgctgctcctagaacatctagccctggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggccctggcgcttcccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactgataa

SEQ ID NO: 178 - DNA, 1281 bp, synthetic construct
  source 1..1281: mol_type = other DNA
atggcgagaggagccgggctcgtgttctttgtgggcgtatgggtcgtttcctgcctggctgccgcacccaggaccagcagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 179 - DNA, 1281 bp, synthetic construct
  source 1..1281: mol_type = other DNA
atggcaagaggagcaggactggtgtttttcgtgggagtttgggtggtgtcttgcctggctgctgctccaagaacctcttctcctggaagaaccatcacagtgggaccaagaggaaatgcaagcaatgcagcaccttctgcttctccaagaaatgcttctgctccaagaaccaccccaacccctcctcagccaagaaaagcaaccaaatccaaagcatccacagcaaaacctgcacctcctccaaaaacaggacctccaaaaacctcctctgaacctgtgagatgcaacagacatgatcctctggcaagatatggatcaagagtgcagatcagatgcagatttccaaattccaccagaacagaattcagactccagatctggagatatgcaacagcaacagatgcagaaattggaacagcaccatctctggaagaagtgatggtgaatgtgtctgctcctcctggaggacagctggtgtatgattctgctccaaacagaacagatcctcatgtgatctgggctgaaggagctggacctggagcttctccaagactgtactctgtggtgggacctctgggaagacagagactgatcattgaagaactgaccctggaaacccagggaatgtactactgggtgtggggaagaacagacagaccttctgcttatggaacctgggtgagagtgagagtgttcagacctccttctctgaccatccaccctcatgctgtgctggaaggacagcctttcaaagcaacctgcacagcagcaacctactaccctggaaacagagctgaatttgtgtggtttgaagatggaagaagggtgtttgatcctgctcagatccacacccagacccaggaaaatcctgatggattttccacagtgtccacagtgacatctgctgctgtgggaggacagggacctccaagaaccttcacctgccagctgacctggcacagagattctgtgtctttttcaagaagaaatgcttctggaacagcttctgtgctgccaagaccaaccatcaccatggaattcacaggagatcatgctgtgtgcacagctggatgtgtgcctgaaggagtgacctttgcttggtttctgggagatgattcttctccagctgaaaaagtggctgtggcttcccagacctcttgtggaagacctggaacagcaaccatcagatccaccctgcctgtgtcttatgaacagacagaatacatttgcagactggctggataccctgatggaatccctgtgctggaacaccactgataa

SEQ ID NO: 180 - DNA, 1278 bp, synthetic construct
  source 1..1278: mol_type = other DNA
atggcgagaggagccggactcgtgttctttgtgggagtctgggttgtgagctgcctggcagcagctccacgcactagctctccaggcagaactatcacagtgggacccagagggaatgccagcaatgcagccccgagtgccagccctcgtaacgccagcgctcccagaacaaccccaactccaccgcagcctagaaaggcgaccaagtccaaagcatccactgcaaaaccagccccacctcccaaaacgggacctcccaagaccagctccgagcctgtaaggtgcaatcggcatgaccccttggcccgatatggcagtcgcgtgcagattcgatgtcggtttcccaactctacccggactgagttccggttgcagatctggaggtatgcgaccgccactgacgctgagatcggcacagcaccaagcctggaagaagtgatggtgaacgttagtgctcctccgggcgggcaactcgtgtatgactccgcacccaaccgcacagatcctcacgtgatttgggccgaaggagccggacccggtgcgtcacctaggctctactctgtcgtaggaccactgggccgtcaacgcctgataatcgaggagctgactctggagacacagggtatgtactactgggtctggggcagaaccgacaggccatctgcttacgggacatgggtccgcgttcgagtatttcggccaccctcactgaccatacatccccatgccgttcttgaagggcagcctttcaaggcaacctgtactgctgccacatactatcccgggaatagggccgagttcgtctggtttgaagatggccgaagggtgtttgacccggctcagatccacacccagacacaggagaaccccgatggcttcagtacggtgtctaccgtcacaagcgccgctgtgggtggccaaggtcctcccagaactttcacctgtcagctgacgtggcacagggattccgtgagcttttcccgccgcaatgcgtcagggaccgcctccgtgcttcctcggccaaccatcacaatggaattcacgggtgatcacgctgtctgcacagctggctgcgttcctgagggcgtgacattcgcatggtttcttggtgacgactcatctcccgcagagaaggtggctgttgcctcacaaacgagttgtgggcgtccaggcactgccaccattcggtccaccctccccgtaagctacgaacagactgagtatatttgcagactggctggatacccggatgggattcctgtcctggaacatcactga

SEQ ID NO: 181 - DNA, 1305 bp, synthetic construct
  source 1..1305: mol_type = other DNA
atgggagtgaccggcattctccagctgcctcgggacagattcaagcggaccagcttcttcctgtgggtcatcatcctgttccagcggaccttcagcatccccagccctggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggccctggcgcttcccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactgataa

SEQ ID NO: 182 - DNA, 1305 bp, synthetic construct
  source 1..1305: mol_type = other DNA
atgggagtgactggcatactccagcttcctagagacaggtttaagcgcacatccttctttctgtgggtcatcatcctgttccaacggaccttcagcattcccagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 183 - DNA, 1305 bp, synthetic construct
  source 1..1305: mol_type = other DNA
atgggagtaaccggaattctccagctgccaagagatcgattcaaaagaacatcatttttcctttgggtaattattctgtttcagagaacattttccatcccttctcctggaagaaccatcacagtgggaccaagaggaaatgcaagcaatgcagcaccttctgcttctccaagaaatgcttctgctccaagaaccaccccaacccctcctcagccaagaaaagcaaccaaatccaaagcatccacagcaaaacctgcacctcctccaaaaacaggacctccaaaaacctcctctgaacctgtgagatgcaacagacatgatcctctggcaagatatggatcaagagtgcagatcagatgcagatttccaaattccaccagaacagaattcagactccagatctggagatatgcaacagcaacagatgcagaaattggaacagcaccatctctggaagaagtgatggtgaatgtgtctgctcctcctggaggacagctggtgtatgattctgctccaaacagaacagatcctcatgtgatctgggctgaaggagctggacctggagcttctccaagactgtactctgtggtgggacctctgggaagacagagactgatcattgaagaactgaccctggaaacccagggaatgtactactgggtgtggggaagaacagacagaccttctgcttatggaacctgggtgagagtgagagtgttcagacctccttctctgaccatccaccctcatgctgtgctggaaggacagcctttcaaagcaacctgcacagcagcaacctactaccctggaaacagagctgaatttgtgtggtttgaagatggaagaagggtgtttgatcctgctcagatccacacccagacccaggaaaatcctgatggattttccacagtgtccacagtgacatctgctgctgtgggaggacagggacctccaagaaccttcacctgccagctgacctggcacagagattctgtgtctttttcaagaagaaatgcttctggaacagcttctgtgctgccaagaccaaccatcaccatggaattcacaggagatcatgctgtgtgcacagctggatgtgtgcctgaaggagtgacctttgcttggtttctgggagatgattcttctccagctgaaaaagtggctgtggcttcccagacctcttgtggaagacctggaacagcaaccatcagatccaccctgcctgtgtcttatgaacagacagaatacatttgcagactggctggataccctgatggaatccctgtgctggaacaccactgataa

SEQ ID NO: 184 - DNA, 1302 bp, synthetic construct
  source 1..1302: mol_type = other DNA
atgggagttaccggaattctgcaattgcccagggatcggttcaagcgcacatccttcttcctgtgggtcatcatcctctttcagcgtactttctccataccctctcccggacgaactatcactgtaggcccaaggggcaacgctagtaatgccgctcccagtgcttcaccacgcaatgcgagcgcacccagaactacacccacacctcctcagccgaggaaagccaccaagtccaaagccagcaccgccaaacccgctcctccacctaaaacagggcctcccaagacctcaagcgagcccgttagatgcaatcggcatgatccactggctcgttacggttctcgggtccagatacgctgtaggtttcctaactccacacgaaccgagttcagattgcagatctggagatatgccacagccactgacgctgagattggcactgcacctagtctggaggaggtgatggtgaacgtgagcgcccctcctggcggtcagcttgtgtatgactcagcaccgaatcgcacagatccgcacgtcatatgggccgaaggtgcagggcccggtgcatcccctcggctgtattccgtggtcggaccactcgggcgccagaggcttatcattgaggaactgaccctcgaaacccagggtatgtactactgggtatggggccgtaccgaccggcctagcgcctacggaacttgggtgagagttcgggtgttcagaccgccaagtcttacaattcaccctcatgccgtgctcgaaggtcagccatttaaggccacgtgtactgcggctacgtactatcccgggaatcgggctgaattcgtgtggtttgaggatggcaggagagtgttcgacccagcccaaatccacacccaaacacaggagaacccagacgggttttccaccgtgtcaacggtcacatctgccgccgtcggaggacaagggccacccagaaccttcacatgccagctgacctggcatagggatagcgtaagctttagccggcgaaacgcatctggaacggcgagcgttctgcctcgaccaacaatcaccatggagttcaccggcgatcacgcagtgtgcactgctgggtgtgtacccgaaggcgtgacatttgcctggtttctgggagatgactcctcacccgcagaaaaggtcgccgttgcatctcagaccagttgtggcaggcccgggactgctaccatccgcagcactctgccggtgtcttacgaacagacggagtacatttgccgcttggcgggctatccagacggcattccagttctggagcatcactga

SEQ ID NO: 185 - DNA, 1233 bp, synthetic construct
  source 1..1233: mol_type = other DNA
atgggcggagctgctgctagactgggagccgtgatcctgttcgtggttatcgtgggactgcatggcgtgcggggcaagtatagaacctcttggaagcgcgtgacaagcggcgaggatgtggttctgcttcctgctcctgccggacctgaggaaagaacaagagcccacaagctgctgtgggccgctgaacctttggatgcctgtggacctctgaggccttcttgggttgcactgtggccacctcggagagtgctggaaacagtggtggatgccgcctgcatgagagcccctgaacctctggccattgcctactctccaccttttccagccggcgacgagggcctgtattctgagcttgcttggagagacagagtggccgtggtcaacgagagcctggttatctatggcgccctggaaaccgacagcggcctgtacacactgtctgtcgtgggcctgtctgacgaggctagacaggtggcatctgtggtgctggtggtggaacctgctccagtgcctacacctacacctgacgactacgacgaggaagatgacgctggcgtcagcgagagaacccctgtttctgtgcctcctcctacgcctcctcgtagacctcctgttgctcctccaacacaccccagagtgatccctgaagtgtctcacgtgcggggcgtgaccgtgcacatggaaacacctgaggccatcctgttcgcccctggcgagacatttggcaccaacgtgtccatccacgctatcgcccacgacgatggcccttacgccatggatgtcgtgtggatgagattcgacgtgcccagcagctgcgccgagatgagaatctacgaggcctgcctgtatcaccctcagctgcccgaatgtctgagccctgctgatgccccttgtgccgttagcagctgggcctatagactggccgtgcggtcttatgccggctgctctagaacaacccctcctcctcggtgtttcgccgaggccagaatggaacctgttcctggactggcctggctggcctccacagtgaacctggaatttcagcacgcctctccacagcacgccggcctgtatctgtgtgtggtgtacgtggacgatcacatccacgcctggggccacatgaccatctctacagccgctcagtaccggaacgccgtggttgaacagcatctgccccagagacagcccgagcctgtggaacctacaagacctcatgttcgggcctgataa

SEQ ID NO: 186 - DNA, 1233 bp, synthetic construct
  source 1..1233: mol_type = other DNA
atgggaggagcagctgccagactcggtgccgtgatcctgttcgtggtcattgttggcctgcacggggtaaggggcaagtaccgcacctcttggaaacgcgttacttccggggaggacgttgtcctccttccagcacccgcaggacctgaggaaaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaggaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctgataa

SEQ ID NO: 187 - DNA, 1233 bp, synthetic construct
  source 1..1233: mol_type = other DNA
atgggaggagctgctgctagattgggagctgtgattctgtttgtggtgattgtgggactgcatggagtgagaggaaaatacagaacctcttggaaaagagtgacctctggagaagatgtggtgctgctgccagctccagctggaccagaagaaagaaccagagcacacaaactgctgtgggctgctgaacctctggatgcttgtggacctctgagaccttcttgggtggctctgtggccaccaagaagggtgctggaaacagtggtggatgctgcttgcatgagagcacctgaacctctggcaattgcatactctcctccttttccagctggagatgaaggactgtattctgaactggcttggagagacagagtggctgtggtgaatgaatctctggtgatctatggagcactggaaacagattctggactgtacaccctgtctgtggtgggactgtctgatgaagcaagacaggtggcatctgtggtgctggtggtggaaccagctccagtgccaaccccaaccccagatgattatgatgaagaagatgatgctggagtgtctgaaagaaccccagtgtctgtgccaccaccaaccccaccaagaagaccaccagtggctccaccaacccacccaagagtgatcccagaagtgtctcatgtgagaggagtgacagtgcacatggaaacccctgaagcaatcctgtttgcacctggagaaacctttggaaccaatgtgtccatccatgcaattgcacatgatgatggaccttatgcaatggatgtggtgtggatgagatttgatgtgccttcttcttgtgctgaaatgagaatctatgaagcttgcctgtaccaccctcagctgcctgaatgcctgtctcctgctgatgctccttgtgctgtgtcttcttgggcttacagactggctgtgagatcttatgctggatgctccagaaccaccccaccaccaagatgctttgctgaagcaagaatggaacctgtgcctggactggcatggctggcatccacagtgaatctggaatttcagcatgcttctcctcagcatgctggactgtacctgtgtgtggtgtatgtggacgatcacatccatgcttggggacacatgaccatcagcacagctgctcagtacagaaatgctgtggtggaacagcacctgccacagagacagccagaaccagtggaaccaaccagaccacatgtgagagcttgataa

SEQ ID NO: 188 - DNA, 1230 bp, synthetic construct
  source 1..1230: mol_type = other DNA
atgggcggagctgcggctcgcctcggagccgtcattctgttcgtggtgatcgtgggtctgcatggggtcagaggaaagtaccggaccagctggaaaagggtgacatctggggaagatgtcgtgctccttcctgcgcctgctggcccagaagaacgcactagggcccacaagcttctgtgggccgcggagccactggatgcgtgtggtcccctgcgaccctcttgggtggcattgtggccaccccgacgcgtactcgaaacggtcgttgacgccgcctgtatgagagcgccagagcccctcgccattgcctacagcccgccttttcccgccggtgacgaaggactgtacagtgagctggcctggcgcgatagggtcgccgtagttaacgagtccctggtgatatacggcgctctggaaaccgacagtggcttgtacaccctgtccgttgtaggcctgtccgatgaagcacggcaagtggcttccgtggtactggtcgtagagcccgcaccagttcccacacccaccccggatgactacgatgaggaggacgatgccggtgtgagtgagcgtacacctgttagcgtacctccaccaactcctccacgccgccctcctgttgcaccgcctacacatccccgtgtgattcctgaagtgtcacacgttagaggggtgacagtccacatggagactcccgaagccatcctctttgcaccaggcgagacttttgggaccaatgtgagcattcacgccatagctcacgatgacgggccctatgccatggacgtggtgtggatgaggttcgatgtgccctcatcatgcgctgagatgcggatctacgaagcttgcctgtatcacccacagcttcccgagtgcttgtctcccgctgacgccccttgtgctgttagctcttgggcttatcggcttgccgtcaggagctatgctggatgctccagaaccacacctccaccgaggtgtttcgccgaggccagaatggagcctgtgccaggactggcctggctggcaagtactgtgaacctggaattccagcacgcatcacctcagcatgcaggcctgtacctctgcgttgtctatgtcgacgaccatatccacgcatggggccatatgaccatcagcacggcagcacagtatcggaatgctgtggtcgagcagcacttgccccagcgacaacccgaaccagtggagccaaccagaccgcatgtgcgggcctga

SEQ ID NO: 189 - RNA, 1284 nt, synthetic construct
  source 1..1284: mol_type = other RNA
atgggcggagctgctgctagactgggagccgtgatcctgttcgtggttatcgtgggactgcatggcgtgcggggcaagtatagccctggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggccctggcgcttcccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactgataa

SEQ ID NO: 190 - RNA, 1284 nt, synthetic construct
  source 1..1284: mol_type = other RNA
atgggaggagcagctgccagactcggtgccgtgatcctgttcgtggtcattgttggcctgcacggggtaaggggcaagtacagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 191 - RNA, 1284 nt, synthetic construct
  source 1..1284: mol_type = other RNA
atgggaggagctgctgctagattgggagctgtgattctgtttgtggtgattgtgggactgcatggagtgagaggaaaatactctcctggaagaaccatcacagtgggaccaagaggaaatgcaagcaatgcagcaccttctgcttctccaagaaatgcttctgctccaagaaccaccccaacccctcctcagccaagaaaagcaaccaaatccaaagcatccacagcaaaacctgcacctcctccaaaaacaggacctccaaaaacctcctctgaacctgtgagatgcaacagacatgatcctctggcaagatatggatcaagagtgcagatcagatgcagatttccaaattccaccagaacagaattcagactccagatctggagatatgcaacagcaacagatgcagaaattggaacagcaccatctctggaagaagtgatggtgaatgtgtctgctcctcctggaggacagctggtgtatgattctgctccaaacagaacagatcctcatgtgatctgggctgaaggagctggacctggagcttctccaagactgtactctgtggtgggacctctgggaagacagagactgatcattgaagaactgaccctggaaacccagggaatgtactactgggtgtggggaagaacagacagaccttctgcttatggaacctgggtgagagtgagagtgttcagacctccttctctgaccatccaccctcatgctgtgctggaaggacagcctttcaaagcaacctgcacagcagcaacctactaccctggaaacagagctgaatttgtgtggtttgaagatggaagaagggtgtttgatcctgctcagatccacacccagacccaggaaaatcctgatggattttccacagtgtccacagtgacatctgctgctgtgggaggacagggacctccaagaaccttcacctgccagctgacctggcacagagattctgtgtctttttcaagaagaaatgcttctggaacagcttctgtgctgccaagaccaaccatcaccatggaattcacaggagatcatgctgtgtgcacagctggatgtgtgcctgaaggagtgacctttgcttggtttctgggagatgattcttctccagctgaaaaagtggctgtggcttcccagacctcttgtggaagacctggaacagcaaccatcagatccaccctgcctgtgtcttatgaacagacagaatacatttgcagactggctggataccctgatggaatccctgtgctggaacaccactgataa

SEQ ID NO: 192 - RNA, 1281 nt, synthetic construct
  source 1..1281: mol_type = other RNA
atgggcggcgcagccgctagactgggtgcagtcatcctctttgtggtgatcgtggggctgcatggggtcagagggaagtattctccgggacggactataaccgtaggtccaagaggaaacgcctctaacgcagccccgtctgcctcaccacgaaacgcctcagctcccagaaccactcctactccaccccagcctaggaaggcgacgaaatccaaggcttccacggccaaacccgcccctccacccaaaaccggacctcctaagaccagctctgaaccggtgcggtgtaataggcacgacccattggcgcgatatggcagtagggtccagatacggtgcagattcccaaacagcacaagaacagaattccggctgcaaatctggcgatatgcaacggccaccgatgccgaaatcggaacagcacccagtctggaagaagtgatggtgaacgtcagtgctccacctggcggacaactggtgtacgactctgcacccaatcgcacagatccccacgtgatttgggccgagggtgctggacctggggcttcacccaggctgtatagcgttgtagggccacttgggaggcagagactcatcattgaggaactgaccctggaaactcagggcatgtactactgggtatggggccgcacagatcgccccagcgcttatggcacctgggtgcgggtgcgggtgtttcgcccaccctccctcaccattcaccctcatgcggttctggagggacagcctttcaaggcaacttgtaccgcagccacctactatcccggcaatagagcggagttcgtctggtttgaggacggccgtagggtgttcgatcctgcccagattcacacccagacacaggagaatcccgacggctttagcacagtgagcactgtgacgtctgctgccgttggtggtcaagggcctcctcgtaccttcacatgccaattgacctggcaccgcgactcagttagctttagccgccggaatgccagtgggaccgccagtgttctcccaaggccgacaatcaccatggagttcactggcgaccatgcagtgtgcacagctgggtgtgtcccagaaggcgtgactttcgcctggtttctgggtgatgactcctcacccgccgagaaagtagctgtcgcttcccagacttcctgtggacgtcctggaactgcgacaatccgaagcacactgccggtttcctacgagcagacggagtacatatgccgccttgcaggctaccccgatggaattccagtccttgagcaccattga

SEQ ID NO: 193 - RNA, 1299 nt, synthetic construct
  source 1..1299: mol_type = other RNA
atgcatcagggcgctccatcttggggtagacgttggttcgttgtgtgggccctgctgggactgacactgggagttctggttgcctctgctgctcctagccctggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggccctggcgcttcccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactgataa

SEQ ID NO: 194 - RNA, 1299 nt, synthetic construct
  source 1..1299: mol_type = other RNA
atgcaccagggtgccccttcctggggcagaaggtggttcgtggtgtgggcccttctggggctgaccctcggagtcctggttgcgagcgcagctcccagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 195 - RNA, 1299 nt, synthetic construct
  source 1..1299: mol_type = other RNA
atgcaccagggagcaccttcttggggaagaagatggtttgtggtgtgggctctgctgggactgaccctgggagtgctggtggcttctgctgctccttctcctggaagaaccatcacagtgggaccaagaggaaatgcaagcaatgcagcaccttctgcttctccaagaaatgcttctgctccaagaaccaccccaacccctcctcagccaagaaaagcaaccaaatccaaagcatccacagcaaaacctgcacctcctccaaaaacaggacctccaaaaacctcctctgaacctgtgagatgcaacagacatgatcctctggcaagatatggatcaagagtgcagatcagatgcagatttccaaattccaccagaacagaattcagactccagatctggagatatgcaacagcaacagatgcagaaattggaacagcaccatctctggaagaagtgatggtgaatgtgtctgctcctcctggaggacagctggtgtatgattctgctccaaacagaacagatcctcatgtgatctgggctgaaggagctggacctggagcttctccaagactgtactctgtggtgggacctctgggaagacagagactgatcattgaagaactgaccctggaaacccagggaatgtactactgggtgtggggaagaacagacagaccttctgcttatggaacctgggtgagagtgagagtgttcagacctccttctctgaccatccaccctcatgctgtgctggaaggacagcctttcaaagcaacctgcacagcagcaacctactaccctggaaacagagctgaatttgtgtggtttgaagatggaagaagggtgtttgatcctgctcagatccacacccagacccaggaaaatcctgatggattttccacagtgtccacagtgacatctgctgctgtgggaggacagggacctccaagaaccttcacctgccagctgacctggcacagagattctgtgtctttttcaagaagaaatgcttctggaacagcttctgtgctgccaagaccaaccatcaccatggaattcacaggagatcatgctgtgtgcacagctggatgtgtgcctgaaggagtgacctttgcttggtttctgggagatgattcttctccagctgaaaaagtggctgtggcttcccagacctcttgtggaagacctggaacagcaaccatcagatccaccctgcctgtgtcttatgaacagacagaatacatttgcagactggctggataccctgatggaatccctgtgctggaacaccactgataa

SEQ ID NO: 196 - RNA, 1296 nt, synthetic construct
  source 1..1296: mol_type = other RNA
atgcatcaaggcgcacccagttggggcagacggtggttcgtagtgtgggccttgctgggactgactctcggtgtcctcgttgcgtctgctgcaccgagtccaggaaggacgattacggtgggacccagaggtaatgcgtccaatgctgcgccatccgcttctccacggaacgcatctgcacccaggactacaccgacaccacctcagccgcgcaaagccaccaagagcaaggccagcacagccaaacccgctcctccacctaaaaccggaccacctaagaccagctctgaacccgtcagatgcaacaggcacgatccgttggccagatatggcagtcgcgtccagatcaggtgtcgcttccctaacagcacacggaccgagttcaggctgcaaatttggcgctacgctacagccactgacgcagagattggcactgctcccagtctggaggaggtcatggtgaacgtgtctgctccaccaggcggtcagctggtctatgactcagcccctaatcgcacagatcctcacgtgatttgggcagaaggtgcggggcctggggcctccccaaggctctactcagtggttggaccccttgggagacagcggctgatcatcgaggaactgactctcgaaacccaaggtatgtactactgggtatggggcagaacagacagaccttcagcttatggcacctgggtgcgggtgagagtgtttaggcctccctccctgacgatccatccccatgctgtgctggaaggacagccgttcaaggcaacatgcacagcagccacttactatcccggaaaccgtgctgagtttgtgtggttcgaggatgggcgacgtgtattcgaccctgcccagattcacacccagacacaggagaatcccgacgggttttccactgtgagcaccgtgacatcagcggcagtaggagggcagggcccaccccgaacgttcacttgccagcttacttggcatcgggacagtgttagctttagccgccggaatgcctctggcaccgcatccgtccttcctcgcccaaccatcaccatggaattcactggcgatcacgccgtttgtacagccgggtgtgttcccgagggagtgacctttgcttggtttctgggcgatgactcaagcccagccgaaaaggtggccgtcgcctcccaaacgagctgtgggcgacctggcaccgctaccatacgtagcactctgcccgtttcctacgaacagaccgagtatatctgccgattggccggttaccccgatgggataccagtcctggagcaccactga

SEQ ID NO: 197 - RNA, 1266 nt, synthetic construct
  source 1..1266: mol_type = other RNA
atgcctggcagatctctgcaaggactggccatcctcggactgtgggtttgcgcaacaggactgagccctggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggccctggcgcttcccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactgataa

SEQ ID NO: 198 - RNA, 1266 nt, synthetic construct
  source 1..1266: mol_type = other RNA
atgcccggcagaagcctccagggactggctatcctggggctgtgggtgtgcgccaccggtcttagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 199 - RNA, 1266 nt, synthetic construct
  source 1..1266: mol_type = other RNA
atgcctggaagatctctccagggactggcaatcctgggactgtgggtgtgtgcaacaggactgtctcctggaagaaccatcacagtgggaccaagaggaaatgcaagcaatgcagcaccttctgcttctccaagaaatgcttctgctccaagaaccaccccaacccctcctcagccaagaaaagcaaccaaatccaaagcatccacagcaaaacctgcacctcctccaaaaacaggacctccaaaaacctcctctgaacctgtgagatgcaacagacatgatcctctggcaagatatggatcaagagtgcagatcagatgcagatttccaaattccaccagaacagaattcagactccagatctggagatatgcaacagcaacagatgcagaaattggaacagcaccatctctggaagaagtgatggtgaatgtgtctgctcctcctggaggacagctggtgtatgattctgctccaaacagaacagatcctcatgtgatctgggctgaaggagctggacctggagcttctccaagactgtactctgtggtgggacctctgggaagacagagactgatcattgaagaactgaccctggaaacccagggaatgtactactgggtgtggggaagaacagacagaccttctgcttatggaacctgggtgagagtgagagtgttcagacctccttctctgaccatccaccctcatgctgtgctggaaggacagcctttcaaagcaacctgcacagcagcaacctactaccctggaaacagagctgaatttgtgtggtttgaagatggaagaagggtgtttgatcctgctcagatccacacccagacccaggaaaatcctgatggattttccacagtgtccacagtgacatctgctgctgtgggaggacagggacctccaagaaccttcacctgccagctgacctggcacagagattctgtgtctttttcaagaagaaatgcttctggaacagcttctgtgctgccaagaccaaccatcaccatggaattcacaggagatcatgctgtgtgcacagctggatgtgtgcctgaaggagtgacctttgcttggtttctgggagatgattcttctccagctgaaaaagtggctgtggcttcccagacctcttgtggaagacctggaacagcaaccatcagatccaccctgcctgtgtcttatgaacagacagaatacatttgcagactggctggataccctgatggaatccctgtgctggaacaccactgataa

SEQ ID NO: 200 - RNA, 1263 nt, synthetic construct
  source 1..1263: mol_type = other RNA
atgcctgggagaagcctgcaagggctcgcaatcttgggcctgtgggtttgtgccacaggcttgtccccgggtcgaacaatcactgttgggcccaggggaaatgccagcaatgctgcaccttcagcaagcccacgaaacgcttcagcacccaggacaacacccactccacctcaaccgcggaaagccaccaagagcaaggcaagtaccgccaaacccgctcctcctcccaagacagggccacccaagacctctagtgagccagtgaggtgtaaccgccatgatccccttgccagatacgggagcagagtgcagattaggtgccggtttccaaactccacgagaaccgaatttcgcctccagatttggcggtatgcgactgccacagacgcagagattggtaccgctcccagcctggaggaggtcatggtgaacgtgtcagcgcctccgggtggccagctggtctacgactctgccccaaatcgaaccgaccctcacgtcatctgggctgaaggagcgggaccaggagcctctccacgcttgtatagcgtagttggccctctggggagacagcgcctgatcattgaggaactgacccttgagacacaggggatgtactactgggtgtggggcaggactgacaggcccagtgcctatggaacttgggttagggtccgcgtctttcggccacccagtctgaccatccatccacatgccgtgctggaaggccagcccttcaaagcgacttgcactgccgccacgtactatccagggaatagagccgagttcgtttggttcgaggatggccggagagtattcgatccagctcagatccacacccagacgcaggaaaacccggacggctttagcacggtgagtaccgtcacctctgctgccgtcggaggccaaggacctccccgtaccttcacatgccagcttacatggcaccgggactcagtaagcttttcacgtcgtaatgcatccggtactgcttctgtgctgcctcgacccaccatcaccatggagttcacaggggatcacgcagtgtgtacggcaggctgcgtgcctgaaggcgtgacattcgcctggtttctcggtgatgactcctctcctgctgaaaaggtggctgtagcctcccaaacaagctgtggtcggcctggaactgccactatacgctccactctcccggtgtcctacgaacagaccgagtacatatgcagactggctggatatcccgatggcattcccgtgctggagcatcactga

SEQ ID NO: 201 - RNA, 1281 nt, synthetic construct
  source 1..1281: mol_type = other RNA
atggctagaggtgccggcctggtgttctttgttggcgtgtgggtcgtgtcctgtctggccgctgctcctagaacatctagccctggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggccctggcgcttcccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactgataa

SEQ ID NO: 202 - RNA, 1281 nt, synthetic construct
  source 1..1281: mol_type = other RNA
atggcgagaggagccgggctcgtgttctttgtgggcgtatgggtcgtttcctgcctggctgccgcacccaggaccagcagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 203 - RNA, 1281 nt, synthetic construct
  source 1..1281: mol_type = other RNA
atggcaagaggagcaggactggtgtttttcgtgggagtttgggtggtgtcttgcctggctgctgctccaagaacctcttctcctggaagaaccatcacagtgggaccaagaggaaatgcaagcaatgcagcaccttctgcttctccaagaaatgcttctgctccaagaaccaccccaacccctcctcagccaagaaaagcaaccaaatccaaagcatccacagcaaaacctgcacctcctccaaaaacaggacctccaaaaacctcctctgaacctgtgagatgcaacagacatgatcctctggcaagatatggatcaagagtgcagatcagatgcagatttccaaattccaccagaacagaattcagactccagatctggagatatgcaacagcaacagatgcagaaattggaacagcaccatctctggaagaagtgatggtgaatgtgtctgctcctcctggaggacagctggtgtatgattctgctccaaacagaacagatcctcatgtgatctgggctgaaggagctggacctggagcttctccaagactgtactctgtggtgggacctctgggaagacagagactgatcattgaagaactgaccctggaaacccagggaatgtactactgggtgtggggaagaacagacagaccttctgcttatggaacctgggtgagagtgagagtgttcagacctccttctctgaccatccaccctcatgctgtgctggaaggacagcctttcaaagcaacctgcacagcagcaacctactaccctggaaacagagctgaatttgtgtggtttgaagatggaagaagggtgtttgatcctgctcagatccacacccagacccaggaaaatcctgatggattttccacagtgtccacagtgacatctgctgctgtgggaggacagggacctccaagaaccttcacctgccagctgacctggcacagagattctgtgtctttttcaagaagaaatgcttctggaacagcttctgtgctgccaagaccaaccatcaccatggaattcacaggagatcatgctgtgtgcacagctggatgtgtgcctgaaggagtgacctttgcttggtttctgggagatgattcttctccagctgaaaaagtggctgtggcttcccagacctcttgtggaagacctggaacagcaaccatcagatccaccctgcctgtgtcttatgaacagacagaatacatttgcagactggctggataccctgatggaatccctgtgctggaacaccactgataa

SEQ ID NO: 204 - RNA, 1278 nt, synthetic construct
  source 1..1278: mol_type = other RNA
atggcgagaggagccggactcgtgttctttgtgggagtctgggttgtgagctgcctggcagcagctccacgcactagctctccaggcagaactatcacagtgggacccagagggaatgccagcaatgcagccccgagtgccagccctcgtaacgccagcgctcccagaacaaccccaactccaccgcagcctagaaaggcgaccaagtccaaagcatccactgcaaaaccagccccacctcccaaaacgggacctcccaagaccagctccgagcctgtaaggtgcaatcggcatgaccccttggcccgatatggcagtcgcgtgcagattcgatgtcggtttcccaactctacccggactgagttccggttgcagatctggaggtatgcgaccgccactgacgctgagatcggcacagcaccaagcctggaagaagtgatggtgaacgttagtgctcctccgggcgggcaactcgtgtatgactccgcacccaaccgcacagatcctcacgtgatttgggccgaaggagccggacccggtgcgtcacctaggctctactctgtcgtaggaccactgggccgtcaacgcctgataatcgaggagctgactctggagacacagggtatgtactactgggtctggggcagaaccgacaggccatctgcttacgggacatgggtccgcgttcgagtatttcggccaccctcactgaccatacatccccatgccgttcttgaagggcagcctttcaaggcaacctgtactgctgccacatactatcccgggaatagggccgagttcgtctggtttgaagatggccgaagggtgtttgacccggctcagatccacacccagacacaggagaaccccgatggcttcagtacggtgtctaccgtcacaagcgccgctgtgggtggccaaggtcctcccagaactttcacctgtcagctgacgtggcacagggattccgtgagcttttcccgccgcaatgcgtcagggaccgcctccgtgcttcctcggccaaccatcacaatggaattcacgggtgatcacgctgtctgcacagctggctgcgttcctgagggcgtgacattcgcatggtttcttggtgacgactcatctcccgcagagaaggtggctgttgcctcacaaacgagttgtgggcgtccaggcactgccaccattcggtccaccctccccgtaagctacgaacagactgagtatatttgcagactggctggatacccggatgggattcctgtcctggaacatcactga

SEQ ID NO: 205 - RNA, 1305 nt, synthetic construct
  source 1..1305: mol_type = other RNA
atgggagtgaccggcattctccagctgcctcgggacagattcaagcggaccagcttcttcctgtgggtcatcatcctgttccagcggaccttcagcatccccagccctggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggccctggcgcttcccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactgataa

SEQ ID NO: 206 - RNA, 1305 nt, synthetic construct
  source 1..1305: mol_type = other RNA
atgggagtgactggcatactccagcttcctagagacaggtttaagcgcacatccttctttctgtgggtcatcatcctgttccaacggaccttcagcattcccagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 207 - RNA, 1305 nt, synthetic construct
  source 1..1305: mol_type = other RNA
atgggagtaaccggaattctccagctgccaagagatcgattcaaaagaacatcatttttcctttgggtaattattctgtttcagagaacattttccatcccttctcctggaagaaccatcacagtgggaccaagaggaaatgcaagcaatgcagcaccttctgcttctccaagaaatgcttctgctccaagaaccaccccaacccctcctcagccaagaaaagcaaccaaatccaaagcatccacagcaaaacctgcacctcctccaaaaacaggacctccaaaaacctcctctgaacctgtgagatgcaacagacatgatcctctggcaagatatggatcaagagtgcagatcagatgcagatttccaaattccaccagaacagaattcagactccagatctggagatatgcaacagcaacagatgcagaaattggaacagcaccatctctggaagaagtgatggtgaatgtgtctgctcctcctggaggacagctggtgtatgattctgctccaaacagaacagatcctcatgtgatctgggctgaaggagctggacctggagcttctccaagactgtactctgtggtgggacctctgggaagacagagactgatcattgaagaactgaccctggaaacccagggaatgtactactgggtgtggggaagaacagacagaccttctgcttatggaacctgggtgagagtgagagtgttcagacctccttctctgaccatccaccctcatgctgtgctggaaggacagcctttcaaagcaacctgcacagcagcaacctactaccctggaaacagagctgaatttgtgtggtttgaagatggaagaagggtgtttgatcctgctcagatccacacccagacccaggaaaatcctgatggattttccacagtgtccacagtgacatctgctgctgtgggaggacagggacctccaagaaccttcacctgccagctgacctggcacagagattctgtgtctttttcaagaagaaatgcttctggaacagcttctgtgctgccaagaccaaccatcaccatggaattcacaggagatcatgctgtgtgcacagctggatgtgtgcctgaaggagtgacctttgcttggtttctgggagatgattcttctccagctgaaaaagtggctgtggcttcccagacctcttgtggaagacctggaacagcaaccatcagatccaccctgcctgtgtcttatgaacagacagaatacatttgcagactggctggataccctgatggaatccctgtgctggaacaccactgataa

SEQ ID NO: 208 - RNA, 1302 nt, synthetic construct
  source 1..1302: mol_type = other RNA
atgggagttaccggaattctgcaattgcccagggatcggttcaagcgcacatccttcttcctgtgggtcatcatcctctttcagcgtactttctccataccctctcccggacgaactatcactgtaggcccaaggggcaacgctagtaatgccgctcccagtgcttcaccacgcaatgcgagcgcacccagaactacacccacacctcctcagccgaggaaagccaccaagtccaaagccagcaccgccaaacccgctcctccacctaaaacagggcctcccaagacctcaagcgagcccgttagatgcaatcggcatgatccactggctcgttacggttctcgggtccagatacgctgtaggtttcctaactccacacgaaccgagttcagattgcagatctggagatatgccacagccactgacgctgagattggcactgcacctagtctggaggaggtgatggtgaacgtgagcgcccctcctggcggtcagcttgtgtatgactcagcaccgaatcgcacagatccgcacgtcatatgggccgaaggtgcagggcccggtgcatcccctcggctgtattccgtggtcggaccactcgggcgccagaggcttatcattgaggaactgaccctcgaaacccagggtatgtactactgggtatggggccgtaccgaccggcctagcgcctacggaacttgggtgagagttcgggtgttcagaccgccaagtcttacaattcaccctcatgccgtgctcgaaggtcagccatttaaggccacgtgtactgcggctacgtactatcccgggaatcgggctgaattcgtgtggtttgaggatggcaggagagtgttcgacccagcccaaatccacacccaaacacaggagaacccagacgggttttccaccgtgtcaacggtcacatctgccgccgtcggaggacaagggccacccagaaccttcacatgccagctgacctggcatagggatagcgtaagctttagccggcgaaacgcatctggaacggcgagcgttctgcctcgaccaacaatcaccatggagttcaccggcgatcacgcagtgtgcactgctgggtgtgtacccgaaggcgtgacatttgcctggtttctgggagatgactcctcacccgcagaaaaggtcgccgttgcatctcagaccagttgtggcaggcccgggactgctaccatccgcagcactctgccggtgtcttacgaacagacggagtacatttgccgcttggcgggctatccagacggcattccagttctggagcatcactga

SEQ ID NO: 209 - RNA, 1233 nt, synthetic construct
  source 1..1233: mol_type = other RNA
atgggcggagctgctgctagactgggagccgtgatcctgttcgtggttatcgtgggactgcatggcgtgcggggcaagtatagaacctcttggaagcgcgtgacaagcggcgaggatgtggttctgcttcctgctcctgccggacctgaggaaagaacaagagcccacaagctgctgtgggccgctgaacctttggatgcctgtggacctctgaggccttcttgggttgcactgtggccacctcggagagtgctggaaacagtggtggatgccgcctgcatgagagcccctgaacctctggccattgcctactctccaccttttccagccggcgacgagggcctgtattctgagcttgcttggagagacagagtggccgtggtcaacgagagcctggttatctatggcgccctggaaaccgacagcggcctgtacacactgtctgtcgtgggcctgtctgacgaggctagacaggtggcatctgtggtgctggtggtggaacctgctccagtgcctacacctacacctgacgactacgacgaggaagatgacgctggcgtcagcgagagaacccctgtttctgtgcctcctcctacgcctcctcgtagacctcctgttgctcctccaacacaccccagagtgatccctgaagtgtctcacgtgcggggcgtgaccgtgcacatggaaacacctgaggccatcctgttcgcccctggcgagacatttggcaccaacgtgtccatccacgctatcgcccacgacgatggcccttacgccatggatgtcgtgtggatgagattcgacgtgcccagcagctgcgccgagatgagaatctacgaggcctgcctgtatcaccctcagctgcccgaatgtctgagccctgctgatgccccttgtgccgttagcagctgggcctatagactggccgtgcggtcttatgccggctgctctagaacaacccctcctcctcggtgtttcgccgaggccagaatggaacctgttcctggactggcctggctggcctccacagtgaacctggaatttcagcacgcctctccacagcacgccggcctgtatctgtgtgtggtgtacgtggacgatcacatccacgcctggggccacatgaccatctctacagccgctcagtaccggaacgccgtggttgaacagcatctgccccagagacagcccgagcctgtggaacctacaagacctcatgttcgggcctgataa

SEQ ID NO: 210 - RNA, 1230 nt, synthetic construct
  source 1..1230: mol_type = other RNA
atgggcggagctgcggctcgcctcggagccgtcattctgttcgtggtgatcgtgggtctgcatggggtcagaggaaagtaccggaccagctggaaaagggtgacatctggggaagatgtcgtgctccttcctgcgcctgctggcccagaagaacgcactagggcccacaagcttctgtgggccgcggagccactggatgcgtgtggtcccctgcgaccctcttgggtggcattgtggccaccccgacgcgtactcgaaacggtcgttgacgccgcctgtatgagagcgccagagcccctcgccattgcctacagcccgccttttcccgccggtgacgaaggactgtacagtgagctggcctggcgcgatagggtcgccgtagttaacgagtccctggtgatatacggcgctctggaaaccgacagtggcttgtacaccctgtccgttgtaggcctgtccgatgaagcacggcaagtggcttccgtggtactggtcgtagagcccgcaccagttcccacacccaccccggatgactacgatgaggaggacgatgccggtgtgagtgagcgtacacctgttagcgtacctccaccaactcctccacgccgccctcctgttgcaccgcctacacatccccgtgtgattcctgaagtgtcacacgttagaggggtgacagtccacatggagactcccgaagccatcctctttgcaccaggcgagacttttgggaccaatgtgagcattcacgccatagctcacgatgacgggccctatgccatggacgtggtgtggatgaggttcgatgtgccctcatcatgcgctgagatgcggatctacgaagcttgcctgtatcacccacagcttcccgagtgcttgtctcccgctgacgccccttgtgctgttagctcttgggcttatcggcttgccgtcaggagctatgctggatgctccagaaccacacctccaccgaggtgtttcgccgaggccagaatggagcctgtgccaggactggcctggctggcaagtactgtgaacctggaattccagcacgcatcacctcagcatgcaggcctgtacctctgcgttgtctatgtcgacgaccatatccacgcatggggccatatgaccatcagcacggcagcacagtatcggaatgctgtggtcgagcagcacttgccccagcgacaacccgaaccagtggagccaaccagaccgcatgtgcgggcctga

SEQ ID NO: 211 - RNA, 1233 nt, synthetic construct
  source 1..1233: mol_type = other RNA
atgggaggagctgctgctagattgggagctgtgattctgtttgtggtgattgtgggactgcatggagtgagaggaaaatacagaacctcttggaaaagagtgacctctggagaagatgtggtgctgctgccagctccagctggaccagaagaaagaaccagagcacacaaactgctgtgggctgctgaacctctggatgcttgtggacctctgagaccttcttgggtggctctgtggccaccaagaagggtgctggaaacagtggtggatgctgcttgcatgagagcacctgaacctctggcaattgcatactctcctccttttccagctggagatgaaggactgtattctgaactggcttggagagacagagtggctgtggtgaatgaatctctggtgatctatggagcactggaaacagattctggactgtacaccctgtctgtggtgggactgtctgatgaagcaagacaggtggcatctgtggtgctggtggtggaaccagctccagtgccaaccccaaccccagatgattatgatgaagaagatgatgctggagtgtctgaaagaaccccagtgtctgtgccaccaccaaccccaccaagaagaccaccagtggctccaccaacccacccaagagtgatcccagaagtgtctcatgtgagaggagtgacagtgcacatggaaacccctgaagcaatcctgtttgcacctggagaaacctttggaaccaatgtgtccatccatgcaattgcacatgatgatggaccttatgcaatggatgtggtgtggatgagatttgatgtgccttcttcttgtgctgaaatgagaatctatgaagcttgcctgtaccaccctcagctgcctgaatgcctgtctcctgctgatgctccttgtgctgtgtcttcttgggcttacagactggctgtgagatcttatgctggatgctccagaaccaccccaccaccaagatgctttgctgaagcaagaatggaacctgtgcctggactggcatggctggcatccacagtgaatctggaatttcagcatgcttctcctcagcatgctggactgtacctgtgtgtggtgtatgtggacgatcacatccatgcttggggacacatgaccatcagcacagctgctcagtacagaaatgctgtggtggaacagcacctgccacagagacagccagaaccagtggaaccaaccagaccacatgtgagagcttgataa

SEQ ID NO: 212 - RNA, 1230 nt, synthetic construct; identical to SEQ ID NO: 210
  source 1..1230: mol_type = other RNA

SEQ ID NO: 213 - protein, 27 aa, synthetic construct
MGGAAARLGAVILFVVIVGLHGVRGKY

SEQ ID NO: 214 - protein, 32 aa, synthetic construct
MHQGAPSWGRRWFVVWALLGLTLGVLVASAAP

SEQ ID NO: 215 - protein, 20 aa, synthetic construct
MPGRSLQGLAILGLWVCATG

SEQ ID NO: 216 - protein, 26 aa, synthetic construct
MARGAGLVFFVGVWVVSCLAAAPRTS

SEQ ID NO: 217 - protein, 34 aa, synthetic construct
MGVTGILQLPRDRFKRTSFFLWVIILFQRTFSIP

SEQ ID NO: 218 - DNA, 81 bp, synthetic construct
  source 1..81: mol_type = other DNA
atgggcggagctgctgctagactgggagccgtgatcctgttcgtggttatcgtgggactgcatggcgtgcggggcaagtat

SEQ ID NO: 219 - DNA, 81 bp, synthetic construct
  source 1..81: mol_type = other DNA
atgggaggagcagctgccagactcggtgccgtgatcctgttcgtggtcattgttggcctgcacggggtaaggggcaagtac

SEQ ID NO: 220 - DNA, 81 bp, synthetic construct
  source 1..81: mol_type = other DNA
atgggaggagctgctgctagattgggagctgtgattctgtttgtggtgattgtgggactgcatggagtgagaggaaaatac

SEQ ID NO: 221 - DNA, 81 bp, synthetic construct
  source 1..81: mol_type = other DNA
atgggcggcgcagccgctagactgggtgcagtcatcctctttgtggtgatcgtggggctgcatggggtcagagggaagtat

SEQ ID NO: 222 - DNA, 81 bp, synthetic construct
  source 1..81: mol_type = other DNA
atgggcggagctgcggctcgcctcggagccgtcattctgttcgtggtgatcgtgggtctgcatggggtcagaggaaagtac

SEQ ID NO: 223 - DNA, 96 bp, synthetic construct
  source 1..96: mol_type = other DNA
atgcatcagggcgctccatcttggggtagacgttggttcgttgtgtgggccctgctgggactgacactgggagttctggttgcctctgctgctcct

SEQ ID NO: 224 - DNA, 96 bp, synthetic construct
  source 1..96: mol_type = other DNA
atgcaccagggtgccccttcctggggcagaaggtggttcgtggtgtgggcccttctggggctgaccctcggagtcctggttgcgagcgcagctccc

SEQ ID NO: 225 - DNA, 96 bp, synthetic construct
  source 1..96: mol_type = other DNA
atgcaccagggagcaccttcttggggaagaagatggtttgtggtgtgggctctgctgggactgaccctgggagtgctggtggcttctgctgctcct

SEQ ID NO: 226 - DNA, 96 bp, synthetic construct
  source 1..96: mol_type = other DNA
atgcatcaaggcgcacccagttggggcagacggtggttcgtagtgtgggccttgctgggactgactctcggtgtcctcgttgcgtctgctgcaccg

SEQ ID NO: 227 - DNA, 63 bp, synthetic construct
  source 1..63: mol_type = other DNA
atgcctggcagatctctgcaaggactggccatcctcggactgtgggtttgcgcaacaggactg

SEQ ID NO: 228 - DNA, 63 bp, synthetic construct
  source 1..63: mol_type = other DNA
atgcccggcagaagcctccagggactggctatcctggggctgtgggtgtgcgccaccggtctt

SEQ ID NO: 229 - DNA, 63 bp, synthetic construct
  source 1..63: mol_type = other DNA
atgcctggaagatctctccagggactggcaatcctgggactgtgggtgtgtgcaacaggactg

SEQ ID NO: 230 - DNA, 63 bp, synthetic construct
  source 1..63: mol_type = other DNA
atgcctgggagaagcctgcaagggctcgcaatcttgggcctgtgggtttgtgccacaggcttg

SEQ ID NO: 231 - DNA, 78 bp, synthetic construct
  source 1..78: mol_type = other DNA
atggctagaggtgccggcctggtgttctttgttggcgtgtgggtcgtgtcctgtctggccgctgctcctagaacatct

SEQ ID NO: 232 - DNA, 78 bp, synthetic construct
  source 1..78: mol_type = other DNA
atggcgagaggagccgggctcgtgttctttgtgggcgtatgggtcgtttcctgcctggctgccgcacccaggaccagc

SEQ ID NO: 233 - DNA, 78 bp, synthetic construct
  source 1..78: mol_type = other DNA
atggcaagaggagcaggactggtgtttttcgtgggagtttgggtggtgtcttgcctggctgctgctccaagaacctct

SEQ ID NO: 234 - DNA, 78 bp, synthetic construct
  source 1..78: mol_type = other DNA
atggcgagaggagccggactcgtgttctttgtgggagtctgggttgtgagctgcctggcagcagctccacgcactagc

SEQ ID NO: 235 - DNA, 102 bp, synthetic construct
  source 1..102: mol_type = other DNA
atgggagtgaccggcattctccagctgcctcgggacagattcaagcggaccagcttcttcctgtgggtcatcatcctgttccagcggaccttcagcatcccc

SEQ ID NO: 236 - DNA, 102 bp, synthetic construct
  source 1..102: mol_type = other DNA
atgggagtgactggcatactccagcttcctagagacaggtttaagcgcacatccttctttctgtgggtcatcatcctgttccaacggaccttcagcattccc

SEQ ID NO: 237 - DNA, 102 bp, synthetic construct
  source 1..102: mol_type = other DNA
atgggagtaaccggaattctccagctgccaagagatcgattcaaaagaacatcatttttcctttgggtaattattctgtttcagagaacattttccatccct

SEQ ID NO: 238 - DNA, 102 bp, synthetic construct
  source 1..102: mol_type = other DNA
atgggagttaccggaattctgcaattgcccagggatcggttcaagcgcacatccttcttcctgtgggtcatcatcctctttcagcgtactttctccataccc

SEQ ID NO: 239 - RNA, 81 nt, synthetic construct
  source 1..81: mol_type = other RNA
atgggcggagctgctgctagactgggagccgtgatcctgttcgtggttatcgtgggactgcatggcgtgcggggcaagtat

SEQ ID NO: 240 - RNA, 81 nt, synthetic construct
  source 1..81: mol_type = other RNA
atgggaggagcagctgccagactcggtgccgtgatcctgttcgtggtcattgttggcctgcacggggtaaggggcaagtac

SEQ ID NO: 241 - RNA, 81 nt, synthetic construct
  source 1..81: mol_type = other RNA
atgggaggagctgctgctagattgggagctgtgattctgtttgtggtgattgtgggactgcatggagtgagaggaaaatac

SEQ ID NO: 242 - RNA, 81 nt, synthetic construct
  source 1..81: mol_type = other RNA
atgggcggcgcagccgctagactgggtgcagtcatcctctttgtggtgatcgtggggctgcatggggtcagagggaagtat

SEQ ID NO: 243 - RNA, 81 nt, synthetic construct
  source 1..81: mol_type = other RNA
atgggcggagctgcggctcgcctcggagccgtcattctgttcgtggtgatcgtgggtctgcatggggtcagaggaaagtac

SEQ ID NO: 244 - RNA, 96 nt, synthetic construct
  source 1..96: mol_type = other RNA
atgcatcagggcgctccatcttggggtagacgttggttcgttgtgtgggccctgctgggactgacactgggagttctggttgcctctgctgctcct

SEQ ID NO: 245 - RNA, 96 nt, synthetic construct
  source 1..96: mol_type = other RNA
atgcaccagggtgccccttcctggggcagaaggtggttcgtggtgtgggcccttctggggctgaccctcggagtcctggttgcgagcgcagctccc

SEQ ID NO: 246 - RNA, 96 nt, synthetic construct
  source 1..96: mol_type = other RNA
atgcaccagggagcaccttcttggggaagaagatggtttgtggtgtgggctctgctgggactgaccctgggagtgctggtggcttctgctgctcct

SEQ ID NO: 247 - RNA, 96 nt, synthetic construct
  source 1..96: mol_type = other RNA
atgcatcaaggcgcacccagttggggcagacggtggttcgtagtgtgggccttgctgggactgactctcggtgtcctcgttgcgtctgctgcaccg

SEQ ID NO: 248 - RNA, 63 nt, synthetic construct
  source 1..63: mol_type = other RNA
atgcctggcagatctctgcaaggactggccatcctcggactgtgggtttgcgcaacaggactg

SEQ ID NO: 249 - RNA, 63 nt, synthetic construct
  source 1..63: mol_type = other RNA
atgcccggcagaagcctccagggactggctatcctggggctgtgggtgtgcgccaccggtctt

SEQ ID NO: 250 - RNA, 63 nt, synthetic construct
  source 1..63: mol_type = other RNA
atgcctggaagatctctccagggactggcaatcctgggactgtgggtgtgtgcaacaggactg

SEQ ID NO: 251 - RNA, 63 nt, synthetic construct
  source 1..63: mol_type = other RNA
atgcctgggagaagcctgcaagggctcgcaatcttgggcctgtgggtttgtgccacaggcttg

SEQ ID NO: 252 - RNA, 78 nt, synthetic construct
  source 1..78: mol_type = other RNA
atggctagaggtgccggcctggtgttctttgttggcgtgtgggtcgtgtcctgtctggccgctgctcctagaacatct

SEQ ID NO: 253 - RNA, 78 nt, synthetic construct
  source 1..78: mol_type = other RNA
atggcgagaggagccgggctcgtgttctttgtgggcgtatgggtcgtttcctgcctggctgccgcacccaggaccagc

SEQ ID NO: 254 - RNA, 78 nt, synthetic construct
  source 1..78: mol_type = other RNA
atggcaagaggagcaggactggtgtttttcgtgggagtttgggtggtgtcttgcctggctgctgctccaagaacctct

SEQ ID NO: 255 - RNA, 78 nt, synthetic construct
  source 1..78: mol_type = other RNA
atggcgagaggagccggactcgtgttctttgtgggagtctgggttgtgagctgcctggcagcagctccacgcactagc

SEQ ID NO: 256 - RNA, 102 nt, synthetic construct
  source 1..102: mol_type = other RNA
atgggagtgaccggcattctccagctgcctcgggacagattcaagcggaccagcttcttcctgtgggtcatcatcctgttccagcggaccttcagcatcccc

SEQ ID NO: 257 - RNA, 102 nt, synthetic construct
  source 1..102: mol_type = other RNA
atgggagtgactggcatactccagcttcctagagacaggtttaagcgcacatccttctttctgtgggtcatcatcctgttccaacggaccttcagcattccc

SEQ ID NO: 258 - RNA, 102 nt, synthetic construct
  source 1..102: mol_type = other RNA
atgggagtaaccggaattctccagctgccaagagatcgattcaaaagaacatcatttttcctttgggtaattattctgtttcagagaacattttccatccct

SEQ ID NO: 259 - RNA, 102 nt, synthetic construct
  source 1..102: mol_type = other RNA
atgggagttaccggaattctgcaattgcccagggatcggttcaagcgcacatccttcttcctgtgggtcatcatcctctttcagcgtactttctccataccc

SEQ ID NO: 260 - protein, 399 aa, Human alphaherpesvirus 2
SPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 261 - DNA, 1203 bp, synthetic construct
  source 1..1203: mol_type = other DNA
agccctggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggccctggcgcttcccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactgataa

SEQ ID NO: 262 - DNA, 1203 bp, synthetic construct
  source 1..1203: mol_type = other DNA
agccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 263 - skipped

SEQ ID NO: 264 - DNA, 1203 bp, synthetic construct
  source 1..1203: mol_type = other DNA
tctcctggaagaaccatcacagtgggaccaagaggaaatgcaagcaatgcagcaccttctgcttctccaagaaatgcttctgctccaagaaccaccccaacccctcctcagccaagaaaagcaaccaaatccaaagcatccacagcaaaacctgcacctcctccaaaaacaggacctccaaaaacctcctctgaacctgtgagatgcaacagacatgatcctctggcaagatatggatcaagagtgcagatcagatgcagatttccaaattccaccagaacagaattcagactccagatctggagatatgcaacagcaacagatgcagaaattggaacagcaccatctctggaagaagtgatggtgaatgtgtctgctcctcctggaggacagctggtgtatgattctgctccaaacagaacagatcctcatgtgatctgggctgaaggagctggacctggagcttctccaagactgtactctgtggtgggacctctgggaagacagagactgatcattgaagaactgaccctggaaacccagggaatgtactactgggtgtggggaagaacagacagaccttctgcttatggaacctgggtgagagtgagagtgttcagacctccttctctgaccatccaccctcatgctgtgctggaaggacagcctttcaaagcaacctgcacagcagcaacctactaccctggaaacagagctgaatttgtgtggtttgaagatggaagaagggtgtttgatcctgctcagatccacacccagacccaggaaaatcctgatggattttccacagtgtccacagtgacatctgctgctgtgggaggacagggacctccaagaaccttcacctgccagctgacctggcacagagattctgtgtctttttcaagaagaaatgcttctggaacagcttctgtgctgccaagaccaaccatcaccatggaattcacaggagatcatgctgtgtgcacagctggatgtgtgcctgaaggagtgacctttgcttggtttctgggagatgattcttctccagctgaaaaagtggctgtggcttcccagacctcttgtggaagacctggaacagcaaccatcagatccaccctgcctgtgtcttatgaacagacagaatacatttgcagactggctggataccctgatggaatccctgtgctggaacaccactgataa

SEQ ID NO: 265 - DNA, 1200 bp, synthetic construct
  source 1..1200: mol_type = other DNA
tctccgggacggactataaccgtaggtccaagaggaaacgcctctaacgcagccccgtctgcctcaccacgaaacgcctcagctcccagaaccactcctactccaccccagcctaggaaggcgacgaaatccaaggcttccacggccaaacccgcccctccacccaaaaccggacctcctaagaccagctctgaaccggtgcggtgtaataggcacgacccattggcgcgatatggcagtagggtccagatacggtgcagattcccaaacagcacaagaacagaattccggctgcaaatctggcgatatgcaacggccaccgatgccgaaatcggaacagcacccagtctggaagaagtgatggtgaacgtcagtgctccacctggcggacaactggtgtacgactctgcacccaatcgcacagatccccacgtgatttgggccgagggtgctggacctggggcttcacccaggctgtatagcgttgtagggccacttgggaggcagagactcatcattgaggaactgaccctggaaactcagggcatgtactactgggtatggggccgcacagatcgccccagcgcttatggcacctgggtgcgggtgcgggtgtttcgcccaccctccctcaccattcaccctcatgcggttctggagggacagcctttcaaggcaacttgtaccgcagccacctactatcccggcaatagagcggagttcgtctggtttgaggacggccgtagggtgttcgatcctgcccagattcacacccagacacaggagaatcccgacggctttagcacagtgagcactgtgacgtctgctgccgttggtggtcaagggcctcctcgtaccttcacatgccaattgacctggcaccgcgactcagttagctttagccgccggaatgccagtgggaccgccagtgttctcccaaggccgacaatcaccatggagttcactggcgaccatgcagtgtgcacagctgggtgtgtcccagaaggcgtgactttcgcctggtttctgggtgatgactcctcacccgccgagaaagtagctgtcgcttcccagacttcctgtggacgtcctggaactgcgacaatccgaagcacactgccggtttcctacgagcagacggagtacatatgccgccttgcaggctaccccgatggaattccagtccttgagcaccattga

SEQ ID NO: 266 - DNA, 1200 bp, synthetic construct
  source 1..1200: mol_type = other DNA
agtccaggaaggacgattacggtgggacccagaggtaatgcgtccaatgctgcgccatccgcttctccacggaacgcatctgcacccaggactacaccgacaccacctcagccgcgcaaagccaccaagagcaaggccagcacagccaaacccgctcctccacctaaaaccggaccacctaagaccagctctgaacccgtcagatgcaacaggcacgatccgttggccagatatggcagtcgcgtccagatcaggtgtcgcttccctaacagcacacggaccgagttcaggctgcaaatttggcgctacgctacagccactgacgcagagattggcactgctcccagtctggaggaggtcatggtgaacgtgtctgctccaccaggcggtcagctggtctatgactcagcccctaatcgcacagatcctcacgtgatttgggcagaaggtgcggggcctggggcctccccaaggctctactcagtggttggaccccttgggagacagcggctgatcatcgaggaactgactctcgaaacccaaggtatgtactactgggtatggggcagaacagacagaccttcagcttatggcacctgggtgcgggtgagagtgtttaggcctccctccctgacgatccatccccatgctgtgctggaaggacagccgttcaaggcaacatgcacagcagccacttactatcccggaaaccgtgctgagtttgtgtggttcgaggatgggcgacgtgtattcgaccctgcccagattcacacccagacacaggagaatcccgacgggttttccactgtgagcaccgtgacatcagcggcagtaggagggcagggcccaccccgaacgttcacttgccagcttacttggcatcgggacagtgttagctttagccgccggaatgcctctggcaccgcatccgtccttcctcgcccaaccatcaccatggaattcactggcgatcacgccgtttgtacagccgggtgtgttcccgagggagtgacctttgcttggtttctgggcgatgactcaagcccagccgaaaaggtggccgtcgcctcccaaacgagctgtgggcgacctggcaccgctaccatacgtagcactctgcccgtttcctacgaacagaccgagtatatctgccgattggccggttaccccgatgggataccagtcctggagcaccactga

SEQ ID NO: 267 - DNA, 1200 bp, synthetic construct
  source 1..1200: mol_type = other DNA
tccccgggtcgaacaatcactgttgggcccaggggaaatgccagcaatgctgcaccttcagcaagcccacgaaacgcttcagcacccaggacaacacccactccacctcaaccgcggaaagccaccaagagcaaggcaagtaccgccaaacccgctcctcctcccaagacagggccacccaagacctctagtgagccagtgaggtgtaaccgccatgatccccttgccagatacgggagcagagtgcagattaggtgccggtttccaaactccacgagaaccgaatttcgcctccagatttggcggtatgcgactgccacagacgcagagattggtaccgctcccagcctggaggaggtcatggtgaacgtgtcagcgcctccgggtggccagctggtctacgactctgccccaaatcgaaccgaccctcacgtcatctgggctgaaggagcgggaccaggagcctctccacgcttgtatagcgtagttggccctctggggagacagcgcctgatcattgaggaactgacccttgagacacaggggatgtactactgggtgtggggcaggactgacaggcccagtgcctatggaacttgggttagggtccgcgtctttcggccacccagtctgaccatccatccacatgccgtgctggaaggccagcccttcaaagcgacttgcactgccgccacgtactatccagggaatagagccgagttcgtttggttcgaggatggccggagagtattcgatccagctcagatccacacccagacgcaggaaaacccggacggctttagcacggtgagtaccgtcacctctgctgccgtcggaggccaaggacctccccgtaccttcacatgccagcttacatggcaccgggactcagtaagcttttcacgtcgtaatgcatccggtactgcttctgtgctgcctcgacccaccatcaccatggagttcacaggggatcacgcagtgtgtacggcaggctgcgtgcctgaaggcgtgacattcgcctggtttctcggtgatgactcctctcctgctgaaaaggtggctgtagcctcccaaacaagctgtggtcggcctggaactgccactatacgctccactctcccggtgtcctacgaacagaccgagtacatatgcagactggctggatatcccgatggcattcccgtgctggagcatcactga

SEQ ID NO: 268 - DNA, 1200 bp, synthetic construct
  source 1..1200: mol_type = other DNA
tctccaggcagaactatcacagtgggacccagagggaatgccagcaatgcagccccgagtgccagccctcgtaacgccagcgctcccagaacaaccccaactccaccgcagcctagaaaggcgaccaagtccaaagcatccactgcaaaaccagccccacctcccaaaacgggacctcccaagaccagctccgagcctgtaaggtgcaatcggcatgaccccttggcccgatatggcagtcgcgtgcagattcgatgtcggtttcccaactctacccggactgagttccggttgcagatctggaggtatgcgaccgccactgacgctgagatcggcacagcaccaagcctggaagaagtgatggtgaacgttagtgctcctccgggcgggcaactcgtgtatgactccgcacccaaccgcacagatcctcacgtgatttgggccgaaggagccggacccggtgcgtcacctaggctctactctgtcgtaggaccactgggccgtcaacgcctgataatcgaggagctgactctggagacacagggtatgtactactgggtctggggcagaaccgacaggccatctgcttacgggacatgggtccgcgttcgagtatttcggccaccctcactgaccatacatccccatgccgttcttgaagggcagcctttcaaggcaacctgtactgctgccacatactatcccgggaatagggccgagttcgtctggtttgaagatggccgaagggtgtttgacccggctcagatccacacccagacacaggagaaccccgatggcttcagtacggtgtctaccgtcacaagcgccgctgtgggtggccaaggtcctcccagaactttcacctgtcagctgacgtggcacagggattccgtgagcttttcccgccgcaatgcgtcagggaccgcctccgtgcttcctcggccaaccatcacaatggaattcacgggtgatcacgctgtctgcacagctggctgcgttcctgagggcgtgacattcgcatggtttcttggtgacgactcatctcccgcagagaaggtggctgttgcctcacaaacgagttgtgggcgtccaggcactgccaccattcggtccaccctccccgtaagctacgaacagactgagtatatttgcagactggctggatacccggatgggattcctgtcctggaacatcactga

SEQ ID NO: 269 - DNA, 1200 bp, synthetic construct
  source 1..1200: mol_type = other DNA
tctcccggacgaactatcactgtaggcccaaggggcaacgctagtaatgccgctcccagtgcttcaccacgcaatgcgagcgcacccagaactacacccacacctcctcagccgaggaaagccaccaagtccaaagccagcaccgccaaacccgctcctccacctaaaacagggcctcccaagacctcaagcgagcccgttagatgcaatcggcatgatccactggctcgttacggttctcgggtccagatacgctgtaggtttcctaactccacacgaaccgagttcagattgcagatctggagatatgccacagccactgacgctgagattggcactgcacctagtctggaggaggtgatggtgaacgtgagcgcccctcctggcggtcagcttgtgtatgactcagcaccgaatcgcacagatccgcacgtcatatgggccgaaggtgcagggcccggtgcatcccctcggctgtattccgtggtcggaccactcgggcgccagaggcttatcattgaggaactgaccctcgaaacccagggtatgtactactgggtatggggccgtaccgaccggcctagcgcctacggaacttgggtgagagttcgggtgttcagaccgccaagtcttacaattcaccctcatgccgtgctcgaaggtcagccatttaaggccacgtgtactgcggctacgtactatcccgggaatcgggctgaattcgtgtggtttgaggatggcaggagagtgttcgacccagcccaaatccacacccaaacacaggagaacccagacgggttttccaccgtgtcaacggtcacatctgccgccgtcggaggacaagggccacccagaaccttcacatgccagctgacctggcatagggatagcgtaagctttagccggcgaaacgcatctggaacggcgagcgttctgcctcgaccaacaatcaccatggagttcaccggcgatcacgcagtgtgcactgctgggtgtgtacccgaaggcgtgacatttgcctggtttctgggagatgactcctcacccgcagaaaaggtcgccgttgcatctcagaccagttgtggcaggcccgggactgctaccatccgcagcactctgccggtgtcttacgaacagacggagtacatttgccgcttggcgggctatccagacggcattccagttctggagcatcactga

SEQ ID NO: 270 - DNA, 1152 bp, synthetic construct
  source 1..1152: mol_type = other DNA
agaacctcttggaagcgcgtgacaagcggcgaggatgtggttctgcttcctgctcctgccggacctgaggaaagaacaagagcccacaagctgctgtgggccgctgaacctttggatgcctgtggacctctgaggccttcttgggttgcactgtggccacctcggagagtgctggaaacagtggtggatgccgcctgcatgagagcccctgaacctctggccattgcctactctccaccttttccagccggcgacgagggcctgtattctgagcttgcttggagagacagagtggccgtggtcaacgagagcctggttatctatggcgccctggaaaccgacagcggcctgtacacactgtctgtcgtgggcctgtctgacgaggctagacaggtggcatctgtggtgctggtggtggaacctgctccagtgcctacacctacacctgacgactacgacgaggaagatgacgctggcgtcagcgagagaacccctgtttctgtgcctcctcctacgcctcctcgtagacctcctgttgctcctccaacacaccccagagtgatccctgaagtgtctcacgtgcggggcgtgaccgtgcacatggaaacacctgaggccatcctgttcgcccctggcgagacatttggcaccaacgtgtccatccacgctatcgcccacgacgatggcccttacgccatggatgtcgtgtggatgagattcgacgtgcccagcagctgcgccgagatgagaatctacgaggcctgcctgtatcaccctcagctgcccgaatgtctgagccctgctgatgccccttgtgccgttagcagctgggcctatagactggccgtgcggtcttatgccggctgctctagaacaacccctcctcctcggtgtttcgccgaggccagaatggaacctgttcctggactggcctggctggcctccacagtgaacctggaatttcagcacgcctctccacagcacgccggcctgtatctgtgtgtggtgtacgtggacgatcacatccacgcctggggccacatgaccatctctacagccgctcagtaccggaacgccgtggttgaacagcatctgccccagagacagcccgagcctgtggaacctacaagacctcatgttcgggcctgataa

SEQ ID NO: 271 - DNA, 1152 bp, synthetic construct
  source 1..1152: mol_type = other DNA
cgcacctcttggaaacgcgttacttccggggaggacgttgtcctccttccagcacccgcaggacctgaggaaaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaggaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctgataa

SEQ ID NO: 272 - DNA, 1152 bp, synthetic construct
  source 1..1152: mol_type = other DNA
agaacctcttggaaaagagtgacctctggagaagatgtggtgctgctgccagctccagctggaccagaagaaagaaccagagcacacaaactgctgtgggctgctgaacctctggatgcttgtggacctctgagaccttcttgggtggctctgtggccaccaagaagggtgctggaaacagtggtggatgctgcttgcatgagagcacctgaacctctggcaattgcatactctcctccttttccagctggagatgaaggactgtattctgaactggcttggagagacagagtggctgtggtgaatgaatctctggtgatctatggagcactggaaacagattctggactgtacaccctgtctgtggtgggactgtctgatgaagcaagacaggtggcatctgtggtgctggtggtggaaccagctccagtgccaaccccaaccccagatgattatgatgaagaagatgatgctggagtgtctgaaagaaccccagtgtctgtgccaccaccaaccccaccaagaagaccaccagtggctccaccaacccacccaagagtgatcccagaagtgtctcatgtgagaggagtgacagtgcacatggaaacccctgaagcaatcctgtttgcacctggagaaacctttggaaccaatgtgtccatccatgcaattgcacatgatgatggaccttatgcaatggatgtggtgtggatgagatttgatgtgccttcttcttgtgctgaaatgagaatctatgaagcttgcctgtaccaccctcagctgcctgaatgcctgtctcctgctgatgctccttgtgctgtgtcttcttgggcttacagactggctgtgagatcttatgctggatgctccagaaccaccccaccaccaagatgctttgctgaagcaagaatggaacctgtgcctggactggcatggctggcatccacagtgaatctggaatttcagcatgcttctcctcagcatgctggactgtacctgtgtgtggtgtatgtggacgatcacatccatgcttggggacacatgaccatcagcacagctgctcagtacagaaatgctgtggtggaacagcacctgccacagagacagccagaaccagtggaaccaaccagaccacatgtgagagcttgataa

SEQ ID NO: 273 - DNA, 1149 bp, synthetic construct
  source 1..1149: mol_type = other DNA
cggaccagctggaaaagggtgacatctggggaagatgtcgtgctccttcctgcgcctgctggcccagaagaacgcactagggcccacaagcttctgtgggccgcggagccactggatgcgtgtggtcccctgcgaccctcttgggtggcattgtggccaccccgacgcgtactcgaaacggtcgttgacgccgcctgtatgagagcgccagagcccctcgccattgcctacagcccgccttttcccgccggtgacgaaggactgtacagtgagctggcctggcgcgatagggtcgccgtagttaacgagtccctggtgatatacggcgctctggaaaccgacagtggcttgtacaccctgtccgttgtaggcctgtccgatgaagcacggcaagtggcttccgtggtactggtcgtagagcccgcaccagttcccacacccaccccggatgactacgatgaggaggacgatgccggtgtgagtgagcgtacacctgttagcgtacctccaccaactcctccacgccgccctcctgttgcaccgcctacacatccccgtgtgattcctgaagtgtcacacgttagaggggtgacagtccacatggagactcccgaagccatcctctttgcaccaggcgagacttttgggaccaatgtgagcattcacgccatagctcacgatgacgggccctatgccatggacgtggtgtggatgaggttcgatgtgccctcatcatgcgctgagatgcggatctacgaagcttgcctgtatcacccacagcttcccgagtgcttgtctcccgctgacgccccttgtgctgttagctcttgggcttatcggcttgccgtcaggagctatgctggatgctccagaaccacacctccaccgaggtgtttcgccgaggccagaatggagcctgtgccaggactggcctggctggcaagtactgtgaacctggaattccagcacgcatcacctcagcatgcaggcctgtacctctgcgttgtctatgtcgacgaccatatccacgcatggggccatatgaccatcagcacggcagcacagtatcggaatgctgtggtcgagcagcacttgccccagcgacaacccgaaccagtggagccaaccagaccgcatgtgcgggcctga

SEQ ID NO: 274 - RNA, 1203 nt, synthetic construct
  source 1..1203: mol_type = other RNA
agccctggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggccctggcgcttcccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactgataa

SEQ ID NO: 275 - RNA, 1203 nt, synthetic construct
  source 1..1203: mol_type = other RNA
agccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 276 - RNA, 1203 nt, synthetic construct
  source 1..1203: mol_type = other RNA
tctcctggaagaaccatcacagtgggaccaagaggaaatgcaagcaatgcagcaccttctgcttctccaagaaatgcttctgctccaagaaccaccccaacccctcctcagccaagaaaagcaaccaaatccaaagcatccacagcaaaacctgcacctcctccaaaaacaggacctccaaaaacctcctctgaacctgtgagatgcaacagacatgatcctctggcaagatatggatcaagagtgcagatcagatgcagatttccaaattccaccagaacagaattcagactccagatctggagatatgcaacagcaacagatgcagaaattggaacagcaccatctctggaagaagtgatggtgaatgtgtctgctcctcctggaggacagctggtgtatgattctgctccaaacagaacagatcctcatgtgatctgggctgaaggagctggacctggagcttctccaagactgtactctgtggtgggacctctgggaagacagagactgatcattgaagaactgaccctggaaacccagggaatgtactactgggtgtggggaagaacagacagaccttctgcttatggaacctgggtgagagtgagagtgttcagacctccttctctgaccatccaccctcatgctgtgctggaaggacagcctttcaaagcaacctgcacagcagcaacctactaccctggaaacagagctgaatttgtgtggtttgaagatggaagaagggtgtttgatcctgctcagatccacacccagacccaggaaaatcctgatggattttccacagtgtccacagtgacatctgctgctgtgggaggacagggacctccaagaaccttcacctgccagctgacctggcacagagattctgtgtctttttcaagaagaaatgcttctggaacagcttctgtgctgccaagaccaaccatcaccatggaattcacaggagatcatgctgtgtgcacagctggatgtgtgcctgaaggagtgacctttgcttggtttctgggagatgattcttctccagctgaaaaagtggctgtggcttcccagacctcttgtggaagacctggaacagcaaccatcagatccaccctgcctgtgtcttatgaacagacagaatacatttgcagactggctggataccctgatggaatccctgtgctggaacaccactgataa

SEQ ID NO: 277 - RNA, 1200 nt, synthetic construct
  source 1..1200: mol_type = other RNA
tctccgggacggactataaccgtaggtccaagaggaaacgcctctaacgcagccccgtctgcctcaccacgaaacgcctcagctcccagaaccactcctactccaccccagcctaggaaggcgacgaaatccaaggcttccacggccaaacccgcccctccacccaaaaccggacctcctaagaccagctctgaaccggtgcggtgtaataggcacgacccattggcgcgatatggcagtagggtccagatacggtgcagattcccaaacagcacaagaacagaattccggctgcaaatctggcgatatgcaacggccaccgatgccgaaatcggaacagcacccagtctggaagaagtgatggtgaacgtcagtgctccacctggcggacaactggtgtacgactctgcacccaatcgcacagatccccacgtgatttgggccgagggtgctggacctggggcttcacccaggctgtatagcgttgtagggccacttgggaggcagagactcatcattgaggaactgaccctggaaactcagggcatgtactactgggtatggggccgcacagatcgccccagcgcttatggcacctgggtgcgggtgcgggtgtttcgcccaccctccctcaccattcaccctcatgcggttctggagggacagcctttcaaggcaacttgtaccgcagccacctactatcccggcaatagagcggagttcgtctggtttgaggacggccgtagggtgttcgatcctgcccagattcacacccagacacaggagaatcccgacggctttagcacagtgagcactgtgacgtctgctgccgttggtggtcaagggcctcctcgtaccttcacatgccaattgacctggcaccgcgactcagttagctttagccgccggaatgccagtgggaccgccagtgttctcccaaggccgacaatcaccatggagttcactggcgaccatgcagtgtgcacagctgggtgtgtcccagaaggcgtgactttcgcctggtttctgggtgatgactcctcacccgccgagaaagtagctgtcgcttcccagacttcctgtggacgtcctggaactgcgacaatccgaagcacactgccggtttcctacgagcagacggagtacatatgccgccttgcaggctaccccgatggaattccagtccttgagcaccattga

SEQ ID NO: 278 - RNA, 1200 nt, synthetic construct
  source 1..1200: mol_type = other RNA
agtccaggaaggacgattacggtgggacccagaggtaatgcgtccaatgctgcgccatccgcttctccacggaacgcatctgcacccaggactacaccgacaccacctcagccgcgcaaagccaccaagagcaaggccagcacagccaaacccgctcctccacctaaaaccggaccacctaagaccagctctgaacccgtcagatgcaacaggcacgatccgttggccagatatggcagtcgcgtccagatcaggtgtcgcttccctaacagcacacggaccgagttcaggctgcaaatttggcgctacgctacagccactgacgcagagattggcactgctcccagtctggaggaggtcatggtgaacgtgtctgctccaccaggcggtcagctggtctatgactcagcccctaatcgcacagatcctcacgtgatttgggcagaaggtgcggggcctggggcctccccaaggctctactcagtggttggaccccttgggagacagcggctgatcatcgaggaactgactctcgaaacccaaggtatgtactactgggtatggggcagaacagacagaccttcagcttatggcacctgggtgcgggtgagagtgtttaggcctccctccctgacgatccatccccatgctgtgctggaaggacagccgttcaaggcaacatgcacagcagccacttactatcccggaaaccgtgctgagtttgtgtggttcgaggatgggcgacgtgtattcgaccctgcccagattcacacccagacacaggagaatcccgacgggttttccactgtgagcaccgtgacatcagcggcagtaggagggcagggcccaccccgaacgttcacttgccagcttacttggcatcgggacagtgttagctttagccgccggaatgcctctggcaccgcatccgtccttcctcgcccaaccatcaccatggaattcactggcgatcacgccgtttgtacagccgggtgtgttcccgagggagtgacctttgcttggtttctgggcgatgactcaagcccagccgaaaaggtggccgtcgcctcccaaacgagctgtgggcgacctggcaccgctaccatacgtagcactctgcccgtttcctacgaacagaccgagtatatctgccgattggccggttaccccgatgggataccagtcctggagcaccactga

SEQ ID NO: 279 - RNA, 1200 nt, synthetic construct
  source 1..1200: mol_type = other RNA
tccccgggtcgaacaatcactgttgggcccaggggaaatgccagcaatgctgcaccttcagcaagcccacgaaacgcttcagcacccaggacaacacccactccacctcaaccgcggaaagccaccaagagcaaggcaagtaccgccaaacccgctcctcctcccaagacagggccacccaagacctctagtgagccagtgaggtgtaaccgccatgatccccttgccagatacgggagcagagtgcagattaggtgccggtttccaaactccacgagaaccgaatttcgcctccagatttggcggtatgcgactgccacagacgcagagattggtaccgctcccagcctggaggaggtcatggtgaacgtgtcagcgcctccgggtggccagctggtctacgactctgccccaaatcgaaccgaccctcacgtcatctgggctgaaggagcgggaccaggagcctctccacgcttgtatagcgtagttggccctctggggagacagcgcctgatcattgaggaactgacccttgagacacaggggatgtactactgggtgtggggcaggactgacaggcccagtgcctatggaacttgggttagggtccgcgtctttcggccacccagtctgaccatccatccacatgccgtgctggaaggccagcccttcaaagcgacttgcactgccgccacgtactatccagggaatagagccgagttcgtttggttcgaggatggccggagagtattcgatccagctcagatccacacccagacgcaggaaaacccggacggctttagcacggtgagtaccgtcacctctgctgccgtcggaggccaaggacctccccgtaccttcacatgccagcttacatggcaccgggactcagtaagcttttcacgtcgtaatgcatccggtactgcttctgtgctgcctcgacccaccatcaccatggagttcacaggggatcacgcagtgtgtacggcaggctgcgtgcctgaaggcgtgacattcgcctggtttctcggtgatgactcctctcctgctgaaaaggtggctgtagcctcccaaacaagctgtggtcggcctggaactgccactatacgctccactctcccggtgtcctacgaacagaccgagtacatatgcagactggctggatatcccgatggcattcccgtgctggagcatcactga

SEQ ID NO: 280 - RNA, 1200 nt, synthetic construct
  source 1..1200: mol_type = other RNA
tctccaggcagaactatcacagtgggacccagagggaatgccagcaatgcagccccgagtgccagccctcgtaacgccagcgctcccagaacaaccccaactccaccgcagcctagaaaggcgaccaagtccaaagcatccactgcaaaaccagccccacctcccaaaacgggacctcccaagaccagctccgagcctgtaaggtgcaatcggcatgaccccttggcccgatatggcagtcgcgtgcagattcgatgtcggtttcccaactctacccggactgagttccggttgcagatctggaggtatgcgaccgccactgacgctgagatcggcacagcaccaagcctggaagaagtgatggtgaacgttagtgctcctccgggcgggcaactcgtgtatgactccgcacccaaccgcacagatcctcacgtgatttgggccgaaggagccggacccggtgcgtcacctaggctctactctgtcgtaggaccactgggccgtcaacgcctgataatcgaggagctgactctggagacacagggtatgtactactgggtctggggcagaaccgacaggccatctgcttacgggacatgggtccgcgttcgagtatttcggccaccctcactgaccatacatccccatgccgttcttgaagggcagcctttcaaggcaacctgtactgctgccacatactatcccgggaatagggccgagttcgtctggtttgaagatggccgaagggtgtttgacccggctcagatccacacccagacacaggagaaccccgatggcttcagtacggtgtctaccgtcacaagcgccgctgtgggtggccaaggtcctcccagaactttcacctgtcagctgacgtggcacagggattccgtgagcttttcccgccgcaatgcgtcagggaccgcctccgtgcttcctcggccaaccatcacaatggaattcacgggtgatcacgctgtctgcacagctggctgcgttcctgagggcgtgacattcgcatggtttcttggtgacgactcatctcccgcagagaaggtggctgttgcctcacaaacgagttgtgggcgtccaggcactgccaccattcggtccaccctccccgtaagctacgaacagactgagtatatttgcagactggctggatacccggatgggattcctgtcctggaacatcactga

SEQ ID NO: 281 - RNA, 1200 nt, synthetic construct
  source 1..1200: mol_type = other RNA
tctcccggacgaactatcactgtaggcccaaggggcaacgctagtaatgccgctcccagtgcttcaccacgcaatgcgagcgcacccagaactacacccacacctcctcagccgaggaaagccaccaagtccaaagccagcaccgccaaacccgctcctccacctaaaacagggcctcccaagacctcaagcgagcccgttagatgcaatcggcatgatccactggctcgttacggttctcgggtccagatacgctgtaggtttcctaactccacacgaaccgagttcagattgcagatctggagatatgccacagccactgacgctgagattggcactgcacctagtctggaggaggtgatggtgaacgtgagcgcccctcctggcggtcagcttgtgtatgactcagcaccgaatcgcacagatccgcacgtcatatgggccgaaggtgcagggcccggtgcatcccctcggctgtattccgtggtcggaccactcgggcgccagaggcttatcattgaggaactgaccctcgaaacccagggtatgtactactgggtatggggccgtaccgaccggcctagcgcctacggaacttgggtgagagttcgggtgttcagaccgccaagtcttacaattcaccctcatgccgtgctcgaaggtcagccatttaaggccacgtgtactgcggctacgtactatcccgggaatcgggctgaattcgtgtggtttgaggatggcaggagagtgttcgacccagcccaaatccacacccaaacacaggagaacccagacgggttttccaccgtgtcaacggtcacatctgccgccgtcggaggacaagggccacccagaaccttcacatgccagctgacctggcatagggatagcgtaagctttagccggcgaaacgcatctggaacggcgagcgttctgcctcgaccaacaatcaccatggagttcaccggcgatcacgcagtgtgcactgctgggtgtgtacccgaaggcgtgacatttgcctggtttctgggagatgactcctcacccgcagaaaaggtcgccgttgcatctcagaccagttgtggcaggcccgggactgctaccatccgcagcactctgccggtgtcttacgaacagacggagtacatttgccgcttggcgggctatccagacggcattccagttctggagcatcactga

SEQ ID NO: 282 - RNA, 1152 nt, synthetic construct
  source 1..1152: mol_type = other RNA
agaacctcttggaagcgcgtgacaagcggcgaggatgtggttctgcttcctgctcctgccggacctgaggaaagaacaagagcccacaagctgctgtgggccgctgaacctttggatgcctgtggacctctgaggccttcttgggttgcactgtggccacctcggagagtgctggaaacagtggtggatgccgcctgcatgagagcccctgaacctctggccattgcctactctccaccttttccagccggcgacgagggcctgtattctgagcttgcttggagagacagagtggccgtggtcaacgagagcctggttatctatggcgccctggaaaccgacagcggcctgtacacactgtctgtcgtgggcctgtctgacgaggctagacaggtggcatctgtggtgctggtggtggaacctgctccagtgcctacacctacacctgacgactacgacgaggaagatgacgctggcgtcagcgagagaacccctgtttctgtgcctcctcctacgcctcctcgtagacctcctgttgctcctccaacacaccccagagtgatccctgaagtgtctcacgtgcggggcgtgaccgtgcacatggaaacacctgaggccatcctgttcgcccctggcgagacatttggcaccaacgtgtccatccacgctatcgcccacgacgatggcccttacgccatggatgtcgtgtggatgagattcgacgtgcccagcagctgcgccgagatgagaatctacgaggcctgcctgtatcaccctcagctgcccgaatgtctgagccctgctgatgccccttgtgccgttagcagctgggcctatagactggccgtgcggtcttatgccggctgctctagaacaacccctcctcctcggtgtttcgccgaggccagaatggaacctgttcctggactggcctggctggcctccacagtgaacctggaatttcagcacgcctctccacagcacgccggcctgtatctgtgtgtggtgtacgtggacgatcacatccacgcctggggccacatgaccatctctacagccgctcagtaccggaacgccgtggttgaacagcatctgccccagagacagcccgagcctgtggaacctacaagacctcatgttcgggcctgataa

SEQ ID NO: 283 - RNA, 1152 nt, synthetic construct
  source 1..1152: mol_type = other RNA
cgcacctcttggaaacgcgttacttccggggaggacgttgtcctccttccagcacccgcaggacctgaggaaaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaggaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctgataa

SEQ ID NO: 284 - RNA, 1152 nt, synthetic construct
  source 1..1152: mol_type = other RNA
agaacctcttggaaaagagtgacctctggagaagatgtggtgctgctgccagctccagctggaccagaagaaagaaccagagcacacaaactgctgtgggctgctgaacctctggatgcttgtggacctctgagaccttcttgggtggctctgtggccaccaagaagggtgctggaaacagtggtggatgctgcttgcatgagagcacctgaacctctggcaattgcatactctcctccttttccagctggagatgaaggactgtattctgaactggcttggagagacagagtggctgtggtgaatgaatctctggtgatctatggagcactggaaacagattctggactgtacaccctgtctgtggtgggactgtctgatgaagcaagacaggtggcatctgtggtgctggtggtggaaccagctccagtgccaaccccaaccccagatgattatgatgaagaagatgatgctggagtgtctgaaagaaccccagtgtctgtgccaccaccaaccccaccaagaagaccaccagtggctccaccaacccacccaagagtgatcccagaagtgtctcatgtgagaggagtgacagtgcacatggaaacccctgaagcaatcctgtttgcacctggagaaacctttggaaccaatgtgtccatccatgcaattgcacatgatgatggaccttatgcaatggatgtggtgtggatgagatttgatgtgccttcttcttgtgctgaaatgagaatctatgaagcttgcctgtaccaccctcagctgcctgaatgcctgtctcctgctgatgctccttgtgctgtgtcttcttgggcttacagactggctgtgagatcttatgctggatgctccagaaccaccccaccaccaagatgctttgctgaagcaagaatggaacctgtgcctggactggcatggctggcatccacagtgaatctggaatttcagcatgcttctcctcagcatgctggactgtacctgtgtgtggtgtatgtggacgatcacatccatgcttggggacacatgaccatcagcacagctgctcagtacagaaatgctgtggtggaacagcacctgccacagagacagccagaaccagtggaaccaaccagaccacatgtgagagcttgataa

SEQ ID NO: 285 - RNA, 1149 nt, synthetic construct
  source 1..1149: mol_type = other RNA
cggaccagctggaaaagggtgacatctggggaagatgtcgtgctccttcctgcgcctgctggcccagaagaacgcactagggcccacaagcttctgtgggccgcggagccactggatgcgtgtggtcccctgcgaccctcttgggtggcattgtggccaccccgacgcgtactcgaaacggtcgttgacgccgcctgtatgagagcgccagagcccctcgccattgcctacagcccgccttttcccgccggtgacgaaggactgtacagtgagctggcctggcgcgatagggtcgccgtagttaacgagtccctggtgatatacggcgctctggaaaccgacagtggcttgtacaccctgtccgttgtaggcctgtccgatgaagcacggcaagtggcttccgtggtactggtcgtagagcccgcaccagttcccacacccaccccggatgactacgatgaggaggacgatgccggtgtgagtgagcgtacacctgttagcgtacctccaccaactcctccacgccgccctcctgttgcaccgcctacacatccccgtgtgattcctgaagtgtcacacgttagaggggtgacagtccacatggagactcccgaagccatcctctttgcaccaggcgagacttttgggaccaatgtgagcattcacgccatagctcacgatgacgggccctatgccatggacgtggtgtggatgaggttcgatgtgccctcatcatgcgctgagatgcggatctacgaagcttgcctgtatcacccacagcttcccgagtgcttgtctcccgctgacgccccttgtgctgttagctcttgggcttatcggcttgccgtcaggagctatgctggatgctccagaaccacacctccaccgaggtgtttcgccgaggccagaatggagcctgtgccaggactggcctggctggcaagtactgtgaacctggaattccagcacgcatcacctcagcatgcaggcctgtacctctgcgttgtctatgtcgacgaccatatccacgcatggggccatatgaccatcagcacggcagcacagtatcggaatgctgtggtcgagcagcacttgccccagcgacaacccgaaccagtggagccaaccagaccgcatgtgcgggcctga

SEQ ID NO: 286 - RNA, 921 nt, synthetic construct
  source 1..921: mol_type = other RNA
aaatatgctctcgctgatccgagcctcaagatggcagatcccaaccgatttcggggaaagaatctgccagtactggaccagctgacggacccacctggcgtcaaacgcgtctaccacatacagcctagtcttgaggacccttttcagccaccgtctatccccattaccgtgtactatgccgtgctggaacgcgcgtgtaggtcagttctgctgcatgccccatccgaagccccccagatcgtcagaggagcttctgatgaagcacgcaaacacacctacaacctcacaatagcgtggtatcgaatgggcgataactgcgcaattcccatcacagtcatggagtacacggagtgcccctacaacaagagcctcggtgtttgccctatcaggacacaacccaggtggagctattacgacagtttcagcgccgtgtctgaggacaatctggggtttctgatgcacgcacccgccttcgagactgccggcacctacttgcggctggtgaagatcaacgactggactgagatcacccagttcatcctggaacatagggccagagccagctgcaagtatgcccttcccctgcggattccgcctgcagcatgtctgacctcaaaagcctaccagcaaggggtgactgtggacagcattggcatgctgcctcgtttcattcccgagaatcaacggacagtggctctgtattccctgaagatcgcaggatggcatgggcccaaaccaccttatacctctacgttgcttccaccagaactcagtgacaccactaatgcgacacagccagaacttgtgcctgaggatcctgaagatagcgctctgttggaggatccagccggtactgtgtcctcccagataccacccaattggcacattccttccattcaggacgtagctccgcatcactga

SEQ ID NO: 287 - DNA, 23 bp, synthetic construct
  source 1..23: mol_type = other DNA
ggcagaagcctactactcggaaa

SEQ ID NO: 288 - DNA, 1200 bp, synthetic construct
  source 1..1200: mol_type = other DNA
agccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggacagacccacatgttatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 289 - DNA, 1200 bp, synthetic construct
  source 1..1200: mol_type = other DNA
agccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 290 - RNA, 1200 nt, synthetic construct
  source 1..1200: mol_type = other RNA
agccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggacagacccacatgttatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 291 - RNA, 1200 nt, synthetic construct
  source 1..1200: mol_type = other RNA
agccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 292 - protein, 23 aa, synthetic construct
MARGAGLVFFVGVWVVSCLVVVP

SEQ ID NO: 293 - DNA, 90 bp, synthetic construct
  source 1..90: mol_type = other DNA
atggggagactcacatccggagtgggcactgctgccctgttggtcgtggccgttggtctgagagttgtgtgtgccaaatatgctctggca

SEQ ID NO: 294 - DNA, 81 bp, synthetic construct
  source 1..81: mol_type = other DNA
atggcactagggagagtgggattagctgtgggtctgtggggactgctctgggtaggagtcgtcgtcgtgctggctaacgca

SEQ ID NO: 295 - DNA, 81 bp, synthetic construct
  source 1..81: mol_type = other DNA
atggccttggggagagtcggccttgcagtgggactgtggggtctgctgtgggttggcgtggtagtcgtgctcgctaacgcc

SEQ ID NO: 296 - DNA, 81 bp, synthetic construct
  source 1..81: mol_type = other DNA
atggctctgggcagagttggactggctgttggactgtggggactgctgtgggttggagtggtggtggtgctggctaatgct

SEQ ID NO: 297 - DNA, 69 bp, synthetic construct
  source 1..69: mol_type = other DNA
atggcacggggcgcaggtttggtctttttcgtgggcgtgtgggtggtgagctgcttggcagccgcacca

SEQ ID NO: 298 - DNA, 69 bp, synthetic construct
  source 1..69: mol_type = other DNA
atggcgagaggagccggactcgtgttctttgtgggagtctgggttgtgagctgcctggcagcagctcca

SEQ ID NO: 299 - DNA, 69 bp, synthetic construct
  source 1..69: mol_type = other DNA
atggcgagaggagccgggctcgtgttctttgtgggcgtatgggtcgtttcctgcctggctgccgcaccc

SEQ ID NO: 300 - DNA, 69 bp, synthetic construct
  source 1..69: mol_type = other DNA
atggcgagaggagccggactcgtgttctttgtgggagtctgggttgtgagctgcctggtagtagttcca

SEQ ID NO: 301 - RNA, 90 nt, synthetic construct
  source 1..90: mol_type = other RNA
atggggagactcacatccggagtgggcactgctgccctgttggtcgtggccgttggtctgagagttgtgtgtgccaaatatgctctggca

SEQ ID NO: 302 - RNA, 81 nt, synthetic construct
  source 1..81: mol_type = other RNA
atggcactagggagagtgggattagctgtgggtctgtggggactgctctgggtaggagtcgtcgtcgtgctggctaacgca

SEQ ID NO: 303 - RNA, 81 nt, synthetic construct
  source 1..81: mol_type = other RNA
atggccttggggagagtcggccttgcagtgggactgtggggtctgctgtgggttggcgtggtagtcgtgctcgctaacgcc

SEQ ID NO: 304 - RNA, 81 nt, synthetic construct
  source 1..81: mol_type = other RNA
atggctctgggcagagttggactggctgttggactgtggggactgctgtgggttggagtggtggtggtgctggctaatgct

SEQ ID NO: 305 - RNA, 69 nt, synthetic construct
  source 1..69: mol_type = other RNA
atggcacggggcgcaggtttggtctttttcgtgggcgtgtgggtggtgagctgcttggcagccgcacca

SEQ ID NO: 306 - RNA, 69 nt, synthetic construct
  source 1..69: mol_type = other RNA
atggcgagaggagccggactcgtgttctttgtgggagtctgggttgtgagctgcctggcagcagctcca

SEQ ID NO: 307 - RNA, 69 nt, synthetic construct
  source 1..69: mol_type = other RNA
atggcgagaggagccgggctcgtgttctttgtgggcgtatgggtcgtttcctgcctggctgccgcaccc

SEQ ID NO: 308 - RNA, 69 nt, synthetic construct
  source 1..69: mol_type = other RNA
atggcgagaggagccggactcgtgttctttgtgggagtctgggttgtgagctgcctggtagtagttcca

SEQ ID NO: 309 - DNA, 1290 bp, synthetic construct
  source 1..1290: mol_type = other DNA
atggggagactcacatccggagtgggcactgctgccctgttggtcgtggccgttggtctgagagttgtgtgtgccaaatatgctctggcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggacagacccacatgttatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 310 - DNA, 1281 bp, synthetic construct
  source 1..1281: mol_type = other DNA
atggcactagggagagtgggattagctgtgggtctgtggggactgctctgggtaggagtcgtcgtcgtgctggctaacgcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 311 - DNA, 1281 bp, synthetic construct
  source 1..1281: mol_type = other DNA
atggccttggggagagtcggccttgcagtgggactgtggggtctgctgtgggttggcgtggtagtcgtgctcgctaacgccagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 312 - DNA, 1281 bp, synthetic construct
  source 1..1281: mol_type = other DNA
atggctctgggcagagttggactggctgttggactgtggggactgctgtgggttggagtggtggtggtgctggctaatgctagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 313 - DNA, 1218 bp, synthetic construct
  source 1..1218: mol_type = other DNA
atggcacggggcgcaggtttggtctttttcgtgggcgtgtgggtggtgagctgcttggcagccgcaccacgcacctcttggaaacgcgttacttccggggaggacgttgtcctccttccagcacccgcaggacctgaggaaaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaggaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 314 - DNA, 1218 bp, synthetic construct
  source 1..1218: mol_type = other DNA
atggcgagaggagccggactcgtgttctttgtgggagtctgggttgtgagctgcctggcagcagctccacgcacctcttggaaacgcgttacttccggggaggacgttgtcctccttccagcacccgcaggacctgaagagaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaagaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 315 - DNA, 1218 bp, synthetic construct
  source 1..1218: mol_type = other DNA
atggcgagaggagccgggctcgtgttctttgtgggcgtatgggtcgtttcctgcctggctgccgcaccccgcacctcttggaaacgcgttacttccggggaggacgttgtcctccttccagcacccgcaggacctgaggaaaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaggaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 316 - DNA, 1218 bp, synthetic construct
  source 1..1218: mol_type = other DNA
atggcgagaggagccggactcgtgttctttgtgggagtctgggttgtgagctgcctggtagtagttccacgcacctcttggaaacgcgttacttccggggaggacgttgtcctccttccagcacccgcaggacctgaagagaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaagaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 317 - DNA, 1152 bp, synthetic construct
  source 1..1152: mol_type = other DNA
atgcgcacctcttggaaacgcgttacttccggggaggacgttgtcctccttccagcacccgcaggacctgaggaaaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaggaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 318 - RNA, 1290 nt, synthetic construct
  source 1..1290: mol_type = other RNA
atggggagactcacatccggagtgggcactgctgccctgttggtcgtggccgttggtctgagagttgtgtgtgccaaatatgctctggcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggacagacccacatgttatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 319 - RNA, 1281 nt, synthetic construct
  source 1..1281: mol_type = other RNA
atggcactagggagagtgggattagctgtgggtctgtggggactgctctgggtaggagtcgtcgtcgtgctggctaacgcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 320 - RNA, 1281 nt, synthetic construct
  source 1..1281: mol_type = other RNA
atggccttggggagagtcggccttgcagtgggactgtggggtctgctgtgggttggcgtggtagtcgtgctcgctaacgccagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 321 - RNA, 1281 nt, synthetic construct
  source 1..1281: mol_type = other RNA
atggctctgggcagagttggactggctgttggactgtggggactgctgtgggttggagtggtggtggtgctggctaatgctagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 322 - RNA, 1218 nt, synthetic construct
  source 1..1218: mol_type = other RNA
atggcacggggcgcaggtttggtctttttcgtgggcgtgtgggtggtgagctgcttggcagccgcaccacgcacctcttggaaacgcgttacttccggggaggacgttgtcctccttccagcacccgcaggacctgaggaaaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaggaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 323 - RNA, 1218 nt, synthetic construct
  source 1..1218: mol_type = other RNA
atggcgagaggagccggactcgtgttctttgtgggagtctgggttgtgagctgcctggcagcagctccacgcacctcttggaaacgcgttacttccggggaggacgttgtcctccttccagcacccgcaggacctgaagagaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaagaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 324 - RNA, 1218 nt, synthetic construct
  source 1..1218: mol_type = other RNA
atggcgagaggagccgggctcgtgttctttgtgggcgtatgggtcgtttcctgcctggctgccgcaccccgcacctcttggaaacgcgttacttccggggaggacgttgtcctccttccagcacccgcaggacctgaggaaaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaggaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 325 - RNA, 1218 nt, synthetic construct
  source 1..1218: mol_type = other RNA
atggcgagaggagccggactcgtgttctttgtgggagtctgggttgtgagctgcctggtagtagttccacgcacctcttggaaacgcgttacttccggggaggacgttgtcctccttccagcacccgcaggacctgaagagaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaagaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 326 - RNA, 1152 nt, synthetic construct
  source 1..1152: mol_type = other RNA
atgcgcacctcttggaaacgcgttacttccggggaggacgttgtcctccttccagcacccgcaggacctgaggaaaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaggaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 327 - protein, 405 aa, synthetic construct
MARGAGLVFFVGVWVVSCLVVVPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRA

SEQ ID NO: 328 - protein, 383 aa, synthetic construct
MRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRA

SEQ ID NO: 329 - DNA, 57 bp, synthetic construct
  source 1..57: mol_type = other DNA
atgcgaatgcagcttctgctgctcattgccttgtcccttgccttggtgaccaactca

SEQ ID NO: 330 - DNA, 1203 bp, synthetic construct
  source 1..1203: mol_type = other DNA
agccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 331 - DNA, 87 bp, synthetic construct; identical to SEQ ID NO: 48
  source 1..87: mol_type = other DNA

SEQ ID NO: 332 - DNA, 87 bp, synthetic construct; identical to SEQ ID NO: 49
  source 1..87: mol_type = other DNA

SEQ ID NO: 333 - DNA, 75 bp, synthetic construct
  source 1..75: mol_type = other DNA
atgggcggagctgctgctagactgggagccgtgatcctgttcgtggttatcgtgggactgcatggcgtgcggggc

SEQ ID NO: 334 - DNA, 924 bp, synthetic construct
  source 1..924: mol_type = other DNA
aaatatgctctcgctgatccgagcctcaagatggcagatcccaaccgatttcggggaaagaatctgccagtactggaccagctgacggacccacctggcgtcaaacgcgtctaccacatacagcctagtcttgaggacccttttcagccaccgtctatccccattaccgtgtactatgccgtgctggaacgcgcgtgtaggtcagttctgctgcatgccccatccgaagccccccagatcgtcagaggagcttctgatgaagcacgcaaacacacctacaacctcacaatagcgtggtatcgaatgggcgataactgcgcaattcccatcacagtcatggagtacacggagtgcccctacaacaagagcctcggtgtttgccctatcaggacacaacccaggtggagctattacgacagtttcagcgccgtgtctgaggacaatctggggtttctgatgcacgcacccgccttcgagactgccggcacctacttgcggctggtgaagatcaacgactggactgagatcacccagttcatcctggaacatagggccagagccagctgcaagtatgcccttcccctgcggattccgcctgcagcatgtctgacctcaaaagcctaccagcaaggggtgactgtggacagcattggcatgctgcctcgtttcattcccgagaatcaacggacagtggctctgtattccctgaagatcgcaggatggcatgggcccaaaccaccttatacctctacgttgcttccaccagaactcagtgacaccactaatgcgacacagccagaacttgtgcctgaggatcctgaagatagcgctctgttggaggatccagccggtactgtgtcctcccagataccacccaattggcacattccttccattcaggacgtagctccgcatcactgataa

SEQ ID NO: 335 - DNA, 1152 bp, synthetic construct; identical to SEQ ID NO: 271
  source 1..1152: mol_type = other DNA

SEQ ID NO: 336 - RNA, 1203 nt, synthetic construct
  source 1..1203: mol_type = other RNA
agccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 337 - DNA, 75 bp, synthetic construct
  source 1..75: mol_type = other DNA
atgggaggagcagctgccagactcggtgccgtgatcctgttcgtggtcattgttggcctgcacggggtaaggggc

SEQ ID NO: 338 - DNA, 75 bp, synthetic construct
  source 1..75: mol_type = other DNA
atgggaggagctgctgctagattgggagctgtgattctgtttgtggtgattgtgggactgcatggagtgagagga

SEQ ID NO: 339 - DNA, 75 bp, synthetic construct
  source 1..75: mol_type = other DNA
atgggcggcgcagccgctagactgggtgcagtcatcctctttgtggtgatcgtggggctgcatggggtcagaggg

SEQ ID NO: 340 - RNA, 924 nt, synthetic construct
  source 1..924: mol_type = other RNA
aaatatgctctcgctgatccgagcctcaagatggcagatcccaaccgatttcggggaaagaatctgccagtactggaccagctgacggacccacctggcgtcaaacgcgtctaccacatacagcctagtcttgaggacccttttcagccaccgtctatccccattaccgtgtactatgccgtgctggaacgcgcgtgtaggtcagttctgctgcatgccccatccgaagccccccagatcgtcagaggagcttctgatgaagcacgcaaacacacctacaacctcacaatagcgtggtatcgaatgggcgataactgcgcaattcccatcacagtcatggagtacacggagtgcccctacaacaagagcctcggtgtttgccctatcaggacacaacccaggtggagctattacgacagtttcagcgccgtgtctgaggacaatctggggtttctgatgcacgcacccgccttcgagactgccggcacctacttgcggctggtgaagatcaacgactggactgagatcacccagttcatcctggaacatagggccagagccagctgcaagtatgcccttcccctgcggattccgcctgcagcatgtctgacctcaaaagcctaccagcaaggggtgactgtggacagcattggcatgctgcctcgtttcattcccgagaatcaacggacagtggctctgtattccctgaagatcgcaggatggcatgggcccaaaccaccttatacctctacgttgcttccaccagaactcagtgacaccactaatgcgacacagccagaacttgtgcctgaggatcctgaagatagcgctctgttggaggatccagccggtactgtgtcctcccagataccacccaattggcacattccttccattcaggacgtagctccgcatcactgataa

SEQ ID NO: 341 - RNA, 1152 nt, synthetic construct; identical to SEQ ID NO: 283
  source 1..1152: mol_type = other RNA

SEQ ID NO: 342 - DNA, 1284 bp, synthetic construct
  source 1..1284: mol_type = other DNA
atgggcggcgcagccgctagactgggtgcagtcatcctctttgtggtgatcgtggggctgcatggggtcagagggaagtatagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 343 - DNA, 1284 bp, synthetic construct
  source 1..1284: mol_type = other DNA
atgggaggagcagctgccagactcggtgccgtgatcctgttcgtggtcattgttggcctgcacggggtaaggggcaagtacagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 344 - DNA, 1299 bp, synthetic construct
  source 1..1299: mol_type = other DNA
atgcatcaaggcgcacccagttggggcagacggtggttcgtagtgtgggccttgctgggactgactctcggtgtcctcgttgcgtctgctgcaccgagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 345 - DNA, 1203 bp, synthetic construct; identical to SEQ ID NO: 330
  source 1..1203: mol_type = other DNA

SEQ ID NO: 346 - DNA, 999 bp, synthetic construct
  source 1..999: mol_type = other DNA
atggggagactcacatcaggcgtaggaaccgctgccctgttggtcgtggccgttggtctgagagttgtgtgtgccaaatatgctctcgctgatccgagcctcaagatggcagatcccaaccgatttcggggaaagaatctgccagtactggaccagctgacggacccacctggcgtcaaacgcgtctaccacatacagcctagtcttgaggacccttttcagccaccgtctatccccattaccgtgtactatgccgtgctggaacgcgcgtgtaggtcagttctgctgcatgccccatccgaagccccccagatcgtcagaggagcttctgatgaagcacgcaaacacacctacaacctcacaatagcgtggtatcgaatgggcgataactgcgcaattcccatcacagtcatggagtacacggagtgcccctacaacaagagcctcggtgtttgccctatcaggacacaacccaggtggagctattacgacagtttcagcgccgtgtctgaggacaatctggggtttctgatgcacgcacccgccttcgagactgccggcacctacttgcggctggtgaagatcaacgactggactgagatcacccagttcatcctggaacatagggccagagccagctgcaagtatgcccttcccctgcggattccgcctgcagcatgtctgacctcaaaagcctaccagcaaggggtgactgtggacagcattggcatgctgcctcgtttcattcccgagaatcaacggacagtggctctgtattccctgaagatcgcaggatggcatgggcccaaaccaccttatacctctacgttgcttccaccagaactcagtgacaccactaatgcgacacagccagaacttgtgcctgaggatcctgaagatagcgctctgttggaggatccagccggtactgtgtcctcccagataccacccaattggcacattccttccattcaggacgtagctccgcatcactgataa

SEQ ID NO: 347 - DNA, 1236 bp, synthetic construct
  source 1..1236: mol_type = other DNA
atggggagactcacatcaggcgtaggaaccgctgccctgttggtcgtggccgttggtctgagagttgtgtgtgccaaatatgctcgcacctcttggaaacgcgttacttccggggaggacgttgtcctccttccagcacccgcaggacctgaggaaaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaggaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctgataa

SEQ ID NO: 348 - RNA, 1284 nt, synthetic construct
  source 1..1284: mol_type = other RNA
atgggcggcgcagccgctagactgggtgcagtcatcctctttgtggtgatcgtggggctgcatggggtcagagggaagtatagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 349 - RNA, 1284 nt, synthetic construct
  source 1..1284: mol_type = other RNA
atgggaggagcagctgccagactcggtgccgtgatcctgttcgtggtcattgttggcctgcacggggtaaggggcaagtacagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 350 - RNA, 1299 nt, synthetic construct
  source 1..1299: mol_type = other RNA
atgcatcaaggcgcacccagttggggcagacggtggttcgtagtgtgggccttgctgggactgactctcggtgtcctcgttgcgtctgctgcaccgagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 351 - RNA, 1203 nt, synthetic construct; identical to SEQ ID NO: 336
  source 1..1203: mol_type = other RNA

SEQ ID NO: 352 - RNA, 999 nt, synthetic construct
  source 1..999: mol_type = other RNA
atggggagactcacatcaggcgtaggaaccgctgccctgttggtcgtggccgttggtctgagagttgtgtgtgccaaatatgctctcgctgatccgagcctcaagatggcagatcccaaccgatttcggggaaagaatctgccagtactggaccagctgacggacccacctggcgtcaaacgcgtctaccacatacagcctagtcttgaggacccttttcagccaccgtctatccccattaccgtgtactatgccgtgctggaacgcgcgtgtaggtcagttctgctgcatgccccatccgaagccccccagatcgtcagaggagcttctgatgaagcacgcaaacacacctacaacctcacaatagcgtggtatcgaatgggcgataactgcgcaattcccatcacagtcatggagtacacggagtgcccctacaacaagagcctcggtgtttgccctatcaggacacaacccaggtggagctattacgacagtttcagcgccgtgtctgaggacaatctggggtttctgatgcacgcacccgccttcgagactgccggcacctacttgcggctggtgaagatcaacgactggactgagatcacccagttcatcctggaacatagggccagagccagctgcaagtatgcccttcccctgcggattccgcctgcagcatgtctgacctcaaaagcctaccagcaaggggtgactgtggacagcattggcatgctgcctcgtttcattcccgagaatcaacggacagtggctctgtattccctgaagatcgcaggatggcatgggcccaaaccaccttatacctctacgttgcttccaccagaactcagtgacaccactaatgcgacacagccagaacttgtgcctgaggatcctgaagatagcgctctgttggaggatccagccggtactgtgtcctcccagataccacccaattggcacattccttccattcaggacgtagctccgcatcactgataa

SEQ ID NO: 353 - RNA, 1236 nt, synthetic construct
  source 1..1236: mol_type = other RNA
atggggagactcacatcaggcgtaggaaccgctgccctgttggtcgtggccgttggtctgagagttgtgtgtgccaaatatgctcgcacctcttggaaacgcgttacttccggggaggacgttgtcctccttccagcacccgcaggacctgaggaaaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaggaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctgataa

SEQ ID NO: 354 - protein, 27 aa, synthetic construct
MGRLTSGVGTAALLVVAVGLRVVCAKY

SEQ ID NO: 355 - protein, 29 aa, synthetic construct
MGRLTSGVGTAALLVVAVGLRVVCAKYAL

SEQ ID NO: 356 - protein, 25 aa, synthetic construct
MGGAAARLGAVILFVVIVGLHGVRG

SEQ ID NO: 357 - protein, 20 aa, synthetic construct
MDRGAVVGFLLGVCVVSCLA

SEQ ID NO: 358 - protein, 20 aa, synthetic construct
MDRGAVVGFLLGVCVVSCLA

SEQ ID NO: 359 - protein, 30 aa, synthetic construct
MHQGAPSWGRRWFVVWALLGLTLGVLVASA

SEQ ID NO: 360 - protein, 22 aa, synthetic construct
MRGGGLICALVVGALVAAVASA

SEQ ID NO: 361 - protein, 18 aa, synthetic construct
MPGRSLQGLAILGLWVCA

SEQ ID NO: 362 - protein, 21 aa, synthetic construct
MPGRSLQGLAILGLWVCATGL

SEQ ID NO: 363 - DNA, 75 bp, synthetic construct
  source 1..75: mol_type = other DNA
atgggcggagctgcggctcgcctcggagccgtcattctgttcgtggtgatcgtgggtctgcatggggtcagagga

SEQ ID NO: 364 - DNA, 90 bp, synthetic construct
  source 1..90: mol_type = other DNA
atgcatcagggcgctccatcttggggtagacgttggttcgttgtgtgggccctgctgggactgacactgggagttctggttgcctctgct

SEQ ID NO: 365 - DNA, 90 bp, synthetic construct
  source 1..90: mol_type = other DNA
atgcaccagggtgccccttcctggggcagaaggtggttcgtggtgtgggcccttctggggctgaccctcggagtcctggttgcgagcgca

SEQ ID NO: 366 - DNA, 90 bp, synthetic construct
  source 1..90: mol_type = other DNA
atgcaccagggagcaccttcttggggaagaagatggtttgtggtgtgggctctgctgggactgaccctgggagtgctggtggcttctgct

SEQ ID NO: 367 - DNA, 90 bp, synthetic construct
  source 1..90: mol_type = other DNA
atgcatcaaggcgcacccagttggggcagacggtggttcgtagtgtgggccttgctgggactgactctcggtgtcctcgttgcgtctgct

SEQ ID NO: 368 - RNA, 87 nt, synthetic construct; identical to SEQ ID NO: 63
  source 1..87: mol_type = other RNA

SEQ ID NO: 369 - RNA, 87 nt, synthetic construct; identical to SEQ ID NO: 64
  source 1..87: mol_type = other RNA

SEQ ID NO: 370 - RNA, 75 nt, synthetic construct
  source 1..75: mol_type = other RNA
atgggcggagctgctgctagactgggagccgtgatcctgttcgtggttatcgtgggactgcatggcgtgcggggc

SEQ ID NO: 371 - RNA, 75 nt, synthetic construct
  source 1..75: mol_type = other RNA
atgggaggagcagctgccagactcggtgccgtgatcctgttcgtggtcattgttggcctgcacggggtaaggggc

SEQ ID NO: 372 - RNA, 75 nt, synthetic construct
  source 1..75: mol_type = other RNA
atgggaggagctgctgctagattgggagctgtgattctgtttgtggtgattgtgggactgcatggagtgagagga

SEQ ID NO: 373 - RNA, 75 nt, synthetic construct
  source 1..75: mol_type = other RNA
atgggcggcgcagccgctagactgggtgcagtcatcctctttgtggtgatcgtggggctgcatggggtcagaggg

SEQ ID NO: 374 - RNA, 75 nt, synthetic construct
  source 1..75: mol_type = other RNA
atgggcggagctgcggctcgcctcggagccgtcattctgttcgtggtgatcgtgggtctgcatggggtcagagga

SEQ ID NO: 375 - RNA, 90 nt, synthetic construct
  source 1..90: mol_type = other RNA
atgcatcagggcgctccatcttggggtagacgttggttcgttgtgtgggccctgctgggactgacactgggagttctggttgcctctgct

SEQ ID NO: 376 - RNA, 90 nt, synthetic construct
  source 1..90: mol_type = other RNA
atgcaccagggtgccccttcctggggcagaaggtggttcgtggtgtgggcccttctggggctgaccctcggagtcctggttgcgagcgca

SEQ ID NO: 377 - RNA, 90 nt, synthetic construct
  source 1..90: mol_type = other RNA
atgcaccagggagcaccttcttggggaagaagatggtttgtggtgtgggctctgctgggactgaccctgggagtgctggtggcttctgct

SEQ ID NO: 378 - RNA, 90 nt, synthetic construct
  source 1..90: mol_type = other RNA
atgcatcaaggcgcacccagttggggcagacggtggttcgtagtgtgggccttgctgggactgactctcggtgtcctcgttgcgtctgct

SEQ ID NO: 379 - RNA, 1287 nt, synthetic construct; identical to SEQ ID NO: 110
  source 1..1287: mol_type = other RNA

SEQ ID NO: 380 - protein, 5 aa, synthetic construct
KYALA

SEQ ID NO: 381 - protein, 4 aa, synthetic construct
LVVV

SEQ ID NO: 382 - protein, 4 aa, synthetic construct
KYAL

SEQ ID NO: 383 - protein, 5 aa, synthetic construct
LVVVP